SEQUENCE LISTING (17 sequences)

SEQ ID NO: 1 - DNA, 20 bp, Artificial Sequence
  Synthetic oligonucleotides accession number U50079
gaaacgtgagggactcagca

SEQ ID NO: 2 - DNA, 20 bp, Artificial Sequence
  Synthetic oligonucleotides accession number U50079
ggaagccagagctggagagg

SEQ ID NO: 3 - DNA, 20 bp, Artificial Sequence
  Synthetic oligonucleotides accession number U50079
gttaggtgaggcactgagga

SEQ ID NO: 4 - DNA, 20 bp, Artificial Sequence
  Synthetic oligonucleotides accession number U31814
gctgagctgttctgatttgg

SEQ ID NO: 5 - DNA, 20 bp, Artificial Sequence
  Synthetic oligonucleotides accession number U31814
cgtgagcacttctcatttcc

SEQ ID NO: 6 - DNA, 20 bp, Artificial Sequence
  Synthetic oligonucleotides accession number AF 039703
cgctttccttgtcattgaca

SEQ ID NO: 7 - DNA, 20 bp, Artificial Sequence
  Synthetic oligonucleotides accession number AF 039703
gcctttcctactcattgtgt

SEQ ID NO: 8 - DNA, 20 bp, Artificial Sequence
  Synthetic oligonucleotides accession number AB 006626
gctgcctgccgtgcccaccc

SEQ ID NO: 9 - DNA, 20 bp, Artificial Sequence
  Synthetic oligonucleotides accession number AB 006626
cgtgcctgcgctgcccacgg

SEQ ID NO: 10 - DNA, 20 bp, Artificial Sequence
  Synthetic oligonucleotides accession number AB 006626
tacagtccatgcaacctcca

SEQ ID NO: 11 - DNA, 20 bp, Artificial Sequence
  Synthetic oligonucleotides accession number AB 006626
atcagtccaaccaacctcgt

SEQ ID NO: 12 - DNA, 20 bp, Artificial Sequence
  Synthetic oligonucleotides accession number AF 039691
cttcggtctcacctgcttgg

SEQ ID NO: 13 - DNA, 20 bp, Artificial Sequence
  Synthetic oligonucleotides accession number AJ 011972
caggctggaatgagctacag

SEQ ID NO: 14 - DNA, 20 bp, Artificial Sequence
  Synthetic oligonucleotides accession number AJ 011972
gacgctgcaatcaggtagac

SEQ ID NO: 15 - DNA, 20 bp, Artificial Sequence
  Synthetic oligonucleotides accession number AF 239243
cttcagccaggatgcccaca

SEQ ID NO: 16 - DNA, 20 bp, Artificial Sequence
  Synthetic oligonucleotides accession number AF 230097
ctccggctcctccatcttcc

SEQ ID NO: 17 - DNA, 20 bp, Artificial Sequence
  Synthetic oligonucleotides accession number AF 230097
agccagctgccacttgatgc

(19) United States
(12) Patent Application Publication
(10) Pub. No.: US 2006/0264415 A1
(43) Pub. Date: Nov. 23, 2006

(54) Inhibitors of histone deacetylase

(75) Inventors: Silvana Marcela Leit de Moradei, Kirkland (CA); Pierre Tessier, Hawkesbury (CA); David Smil, Montreal (CA); Amal Wahhab, Laval (CA); Robert Deziel, Mount-Royal (CA); Sukhdev Manku, L'lle Perrot (CA); John Mancuso, Vaudreuil (CA); Eric Therrien, Laval (CA); Martin Allan, Montreal (CA); Yves Andre Chantigny, Pincourt (CA); Alain Ajamian, Montreal (CA); Patrick Beaulieu, Laval (CA)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: MethylGene Inc.

(21) Appl. No.: 11/395,173

(22) Filed: Mar. 31, 2006

Related U.S. Application Data
(60) Provisional application No. 60/667,708, filed on Apr. 1, 2005.

(51) Int. Cl. A61K 31/541; A61K 31/5377; A61K 31/496; A61K 31/454; A61K 31/397; C07D 417/02; C07D 413/02; C07D 403/02

(52) U.S. Cl. 514/210.18; 514/227.5; 514/235.2; 514/255.01; 514/252.13; 514/317; 544/60; 544/143; 544/359; 544/360; 544/373; 546/207; 546/229; 548/950; 548/953

(57) ABSTRACT

This invention relates to compounds for the inhibition of histone deacetylase. More particularly, the invention provides for compounds of formula (I) 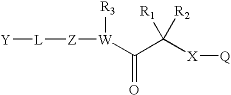
wherein Y, L, Z, W, X, Q, R₁, R₂ and R₃ are as defined in the specification.

### CROSS-REFERENCE TO RELATED APPLICATION

This application claims priority from U.S. provisional application Ser. No. 60/667,708, filed Apr. 1, 2005, which is incorporated herein by reference in its entirety.

### BACKGROUND OF THE INVENTION

1. Field of the Invention

This invention relates to compounds for the inhibition of histone deacetylase.

2. Description of Related Art

In eukaryotic cells, nuclear DNA associates with histones to form a compact complex called chromatin. The histones constitute a family of basic proteins which are generally highly conserved across eukaryotic species. The core histones, termed H2A, H2B, H3, and H4, associate to form a protein core. DNA winds around this protein core, with the basic amino acids of the histones interacting with the negatively charged phosphate groups of the DNA. Approximately 146 base pairs of DNA wrap around a histone core to make up a nucleosome particle, the repeating structural motif of chromatin.

Csordas, Biochem. J., 286: 23-38 (1990) teaches that histones are subject to posttranslational acetylation of the α,ε-amino groups of N-terminal lysine residues, a reaction that is catalyzed by histone acetyl transferase (HAT1). Acetylation neutralizes the positive charge of the lysine side chain, and is thought to impact chromatin structure. Indeed, Taunton et al., Science, 272: 408-411 (1996), teaches that access of transcription factors to chromatin templates is enhanced by histone hyperacetylation. Taunton et al. further teaches that an enrichment in underacetylated histone H4 has been found in transcriptionally silent regions of the genome.

Histone acetylation is a reversible modification, with deacetylation being catalyzed by a family of enzymes termed histone deacetylases (HDACs). The molecular cloning of gene sequences encoding proteins with HDAC activity has established the existence of a set of discrete HDAC enzyme isoforms. Grozinger et al., Proc. Natl. Acad. Sci. USA, 96:4868-4873 (1999), teaches that HDACs may be divided into two classes, the first represented by yeast Rpd3-like proteins, and the second represented by yeast Hd-like proteins. Grozinger et al. also teaches that the human HDAC-1, HDAC-2, and HDAC-3 proteins are members of the first class of HDACs, and discloses new proteins, named HDAC-4, HDAC-5, and HDAC-6, which are members of the second class of HDACs. Kao et al., Gene & Development 14:55-66 (2000), discloses an additional member of this second class, called HDAC-7. More recently, Hu, E. et al. J. Bio. Chem. 275:15254-13264 (2000) disclosed another member of the first class of histone deacetylases, HDAC-8. Zhou et al., Proc. Natl. Acad. Sci. U.S.A., 98: 10572-10577 (2001) teaches the cloning and characterization of a new histone deacetylase, HDAC-9. Kao et al., J. Biol. Chem., 277:187-93 (2002) teaches the isolation and characterization of mammalian HDAC10, a novel histone deacetylase. Gao et al, J. Biol. Chem. (In press) teaches the cloning and functional characterization of HDAC11, a novel member of the human histone deacetylase family. Shore, Proc. Natl. Acad. Sci. U.S.A. 97: 14030-2 (2000) discloses a third class of deacetylase activity, the Sir2 protein family. It has been unclear what roles these individual HDAC enzymes play.

Studies utilizing known HDAC inhibitors have established a link between acetylation and gene expression. Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., TIBS 25: March (2000), discloses HDAC as a pervasive feature of transcriptional repressor systems. Magnaghi-Jaulin et al., Prog. Cell Cycle Res. 4:4147 (2000), discloses HDAC as a transcriptional co-regulator important for cell cycle progression.

Richon et al., Proc. Natl. Acad. Sci. USA, 95: 3003-3007 (1998), discloses that HDAC activity is inhibited by trichostatin A (TSA), a natural product isolated from Streptomyces hygroscopicus, which has been shown to inhibit histone deacetylase activity and arrest cell cycle progression in cells in the G1 and G2 phases (Yoshida et al., J. Biol. Chem. 265: 17174-17179, 1990; Yoshida et al., Exp. Cell Res. 177: 122-131, 1988), and by a synthetic compound, suberoylanilide hydroxamic acid (SAHA). Yoshida and Beppu, Exper. Cell Res., 177: 122-131 (1988), teaches that TSA causes arrest of rat fibroblasts at the G₁ and G₂ phases of the cell cycle, implicating HDAC in cell cycle regulation. Indeed, Finnin et al., Nature, 401: 188-193 (1999), teaches that TSA and SAHA inhibit cell growth, induce terminal differentiation, and prevent the formation of tumors in mice. Suzuki et al., U.S. Pat. No. 6,174,905, EP 0847992, JP 258863/96, and Japanese Application No. 10138957, disclose benzamide derivatives that induce cell differentiation and inhibit HDAC. Delorme et al., WO 01/38322 and PCT/IB01/00683, disclose additional compounds that serve as HDAC inhibitors. Other inhibitors of histone deacetylase activity, including trapoxin, depudecin, FR901228 (Fujisawa Pharmaceuticals), and butyrate, have been found to similarly inhibit cell cycle progression in cells (Taunton et al., Science 272: 408-411, 1996; Kijima et al., J. Biol. Chem. 268(30):22429-22435, 1993; Kwon et al., Proc. Natl. Acad. Sci. USA 95(7):3356-61, 1998).

These findings suggest that inhibition of HDAC activity represents a novel approach for intervening in cell cycle regulation and that HDAC inhibitors have great therapeutic potential in the treatment of cell proliferative diseases or conditions. To date, few inhibitors of histone deacetylase are known in the art. It would be highly desirable to have inhibiters of histone deacetylase.

### SUMMARY OF THE INVENTION

The present invention provides compounds for the inhibition of histone deacetylase.

In a first aspect, the present invention provides compounds that are useful as inhibitors of histone deacetylase that have the formula

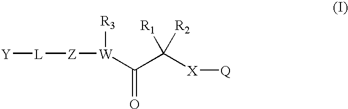

wherein Y, L, Z, W, X, Q, R₁, R₂ and R₃ are as defined below.

In a second aspect, the invention provides a composition comprising a compound according to the first aspect and a pharmaceutically acceptable carrier.

In a third aspect, the invention provides a method of inhibiting histone deacetylase, the method comprising contacting the histone deacetylase or a cell containing histone deacetylase, with an inhibiting effective amount of a compound according to the first aspect or a composition according to second aspect.

The foregoing merely summarizes the one aspect of the invention and is not intended to be limiting in nature. These aspects and other aspects and embodiments are described more fully below. The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

### DETAILED DESCRIPTION OF THE INVENTION

The present invention provides compounds that are useful as inhibitors of histone deacetylase.

In one aspect, the invention provides compound of the formula 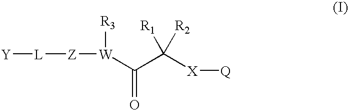
or pharmaceutically acceptable salts thereof, wherein
W is nitrogen or oxygen, wherein when W is oxygen, R₃ is absent;
X is a covalent bond, —S—, —SO—, —SO₂—, —O—, —NR₃—, —CH₂—, optionally substituted C₁-C₆ alkyl, or a structure of the formula 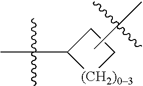
R₁ and R₂ are independently selected from the group consisting of —H, C₁-C₆ alkyl, halo, —N(H)—C(O)—O—C₁-C₆ alkyl, —N(H)—C(O)—O-benzyl, C₃-C₆ cycloalkyl, aryl, aryl-C₁-C₆ alkyl-, and heteroaryl-C₁-C₆ alkyl, wherein the alkyl, benzyl, cycloalkyl, aryl and heteroaryl moieties of said R₁ and R₂ are optionally substituted; or
R₁ and R₂ together with the carbon atom to which they are attached form a 3 to 9-membered heterocyclyl-aryl, C₃-C₆-cycloalkyl or 3 to 9-membered heterocyclyl group, wherein each of the cycloalkyl, heterocyclyl and heterocyclyl-aryl is optionally substituted with one or more groups selected from oxo, —OH, —CN, C₁-C₆ alkyl, C₁-C₆ alkoxy, —NO₂, —N(R₃)(R₃ₐ), halo, —SH, mono- to per-halogenated C₁-C₆ alkyl; or
when X-Q is absent, R₁ and R₂ together with the atom to which they are attached form an aryl, heterocyclyl, cycloalkyl or heteroaryl group, wherein said aryl, heterocyclyl, cycloalkyl and heteroaryl are optionally substituted, and wherein R³ is optionally connected to the aryl, heterocyclyl, cycloalkyl or heteroaryl by a covalent bond;
X-Q, R₃ and R₃ₐ are independently selected from the group consisting of —H, —OH, —C(O)H, heterocyclyl, C₁-C₆alkyl, C₂-C₆alkenyl, C₂-C₃alkynyl, C₂-C₄ alkyl-OR₃, C₂-C₆ hydroxyalkyl, heteroaryl, C₁-C₆heteroalkyl-aryl, C₀-C₆alkylheteroaryl, C₀-C₆heteroalkylheteroaryl, C₁-C₃alkyl-C(O)NR₃-heteroaryl, C₁-C₃alkyl-C(O)NR₃-aryl, C₁-C₄alkyl-C(O)OR₃, —C₁-C₆ hydroxyalkyl-C(O)—OH, —C(O)—NH-aryl, —C(O)CF₃, —C(O)—NH₂, —CH(NH₂)—C(O)—OH, —NH₂, —CH(NH₂)—C(O)—O—C₁-C₆ alkyl, —C(O)—OH, —C(O)—O—C₁-C₆ alkyl, C₁-C₆ heteroalkyl-C₁-C₆ alkyl-, C₃-C₆ cycloalkyl, heterocyclyl-C₁-C₆ alkyl-, —C₁-C₆ alkylaryl, aryl, —C₀-C₆ alkyl-S(O)—C₁-C₆ alkylaryl, —C₀-C₆ alkyl-O—C₀-C₆ alkylaryl, —C₀-C₆ alkyl-S—C₀-C₆ alkylaryl, —C₀-C₆ alkyl-S—C₀-C₆ alkylheteroaryl, —C₀-C₆ alkyl-S—C₁-C₆ alkyl-C(O)—OH, —C₀-C₆ alkyl-S—C₁-C₆ hydroxyalkyl-C(O)—O-C₁-C₆ alkyl, —C₀-C₆ alkyl-S—C₁-C₆ alkyl-CH(NH₂)—C(O)—OR₄, —C₀-C₆ alkyl-S—C₁-C₆ alkyl-OH, —C₀-C₆ alkyl-S—C₁-C₆ alkyl-C(O)—O-C₁-C₆ alkyl, —C₀-C₆ alkyl-S(O)-C₁-C₆ alkyl-C(O)—OR₄, —C₀-C₆ alkyl-S(O)-C₁-C₆ alkyl-C(O)—N(R₄)-aryl, —C₀-C₆ alkyl-S—C₁-C₆ alkyl-C(O)—N(R₄)(R₄ₐ), —C₀-C₆ alkyl-S—C₁-C₆ alkyl-N(R₄)(R₄ₐ), —C₁-C₆ alkylheteroaryl and heteroaryl, wherein each alkyl, alkenyl, alkynyl, heteroalkyl, heteroalkyl, cycloalkyl, heterocyclyl, aryl and heteroaryl moiety of the aforementioned X-Q, R³ and R^{3a} is optionally substituted;
Q is selected from the group consisting of —H, —OH, —N(R₃)(R₃ₐ), halo, —SH, —C(O)OR₃, —C(O)R₃, —C₀-C₃alkyl-diphenyl-R₄, —C₀-C₃alkyl-aryl-, —C₀-C₃alkyl-heteoraryl, —C₁-C₆ alkyl-N(R₃)—C(O)—C₁-C₆ alkyl-B—(CH₂)ₙ—R₃, —C₁-C₆-alkyl-N(R₃)—C(O)—C₁-C₆ alkyl-O—C₁-C₆ alkyl-R₃, —C₁-C₆-alkyl-C(O)—OR₃, —C₁-C₆-alkyl-N(R₃)(R₃ₐ), —C₁-C₆-alkyl-CN, —C₁-C₆ alkyl-C(O)—N(R₃)—N(R₃)-aryl, —C₁-C₆ alkyl-N(R₃)—C₁-C₆ heteroalkyl, —C₁-C₆ alkyl-N(R₃)—C₃-C₆ cycloalkyl, —C₁-C₆ alkyl-N(R₃)-heterocyclyl, —C₁-C₆ alkyl-N(R₃)-aryl, —C₁-C₆ alkyl-N(R₃)—C₁-C₆-alkyl-aryl, —C₁-C₆ alkyl-N(R₃)-heteroaryl, —C₁-C₆ alkyl-N(R₃)—C₃-C₆ cycloalkyl, C₁-C₆ alkyl substituted with —OH, —C₁-C₆ alkyl-O-C₁-C₆ alkyl, —C₁-C₆ alkyl-O—C₃-C₆ cycloalkyl, —C₁-C₆-alkyl-O—C₁-C₆ alkyl-(C₁-C₆ heteroalkyl), —C₁-C₆-alkyl-O—C₁-C₆ alkyl-heterocyclyl, —C₁-C₆ alkyl-O-aryl, —C₁-C₆-alkyl-O-C₁-C₆ alkylaryl, —C₁-C₆ alkyl-O-heteroaryl, —C₁-C₆ alkylhydroxamate, C₁-C₆ alkyl, —C₁-C₆ alkyl-(C₁-C₆ heteroalkyl), C₃-C₆ cycloalkyl, —C₁-C₆ alkylheterocyclyl, —C₁-C₆ alkyl-C₂-C₆ alkenyl, —C₁-C₆ alkyl-C₂-C₆ alkynyl, aryl, —C₁-C₆ alkylaryl, heteroaryl, C₁-C₆ alkylheteroaryl, C₁-C₃ alkyl-CN, —C₁-C₆ alkyl-CH(OR₃)—C(O)—OR₃, —C₁-C₆ alkyl-CH(N(R₃)(R₃ₐ))—C(O)—OR₃, —C₁-C₆ alkyl-C(O)—N(R₃)(R₃ₐ), —C₁-C₆ alkyl-N(R₃)—C(O)—N(R₃)(R₃ₐ), —C₁-C₆ alkyl-N(R₃)—C(O)—OR₃, —C₁-C₆ alkyl-N(R₃)—C(O)—R₃, —C₁-C₆ alkyl-N(R₃)—S(O)₂—R₃, —C₁-C₆ alkyl-S(O)₂—N(R₃)(R₃ₐ), —C₁-C₆ alkyl-CH(N(R₃)(R₃ₐ))—C₁-C₆ alkyl-OR₃, or —C₁-C₆ alkyl-O—C(O)—N(R₃)(R₃ₐ), —C₁-C₆ alkyl-(C═NR₃)—N(R₃)(R₃ₐ), —C₁-C₆ alkyl-X—C₁-C₆ alkyl-C(O)OR₃, —C₁-C₆ alkyl-X—C₁-C₆ alkyl-OR₃, and —C₁-C₆ alkyl-X—C₁-C₆ alkyl-N(R₃)(R₃ₐ), C₁-C₄ alkyl-aryl- wherein the C₁-C₄ alkyl is optionally substituted with —C₁-C₄ alkylOR₃, C₁-C₄ alkylNR₃, R₃ₐ, C₀-C₄ alkylC(O)N(R₃)(R₃ₐ) or C₀-C₄ alkylC(O)OR₃, C₁-C₄ alkyl-heteroaryl- wherein the C₁-C₄ alkyl is optionally substituted with —C₁-C₄ alkylOR₃, C₁-C₄ alkylN(R₃)(R₃ₐ), C₀-C₄ alkylC(O)N(R₃)(R₃ₐ) or C₀-C₄ alkylC(O)OR₃, C₂-C₄ alkenyl, C₂-C₄ alkynyl, C₀-C₆ alkylC(O)NR₃—NR₃aryl and C₀-C₆ alkylC(O)NR₃—NR₃heteroaryl, wherein each alkyl, alkenyl, alkynyl, heteroalkyl, heteroalkyl, cycloalkyl, heterocyclyl, aryl and heteroaryl moiety of the aforementioned Q is optionally substituted;
B is selected from the group consisting of —O—, —S(O)—, —S— and —S(O)₂—,
n is 0 or an interger from 1 to 3;
R₄ and R₄ₐ are independently selected from the group consisting of —H, C₁-C₆ alkyl, C₂-C₆alkenyl, C₂-C₆alkynyl, C₁-C₆ alkyl-R₃, —C₀-C₆ alkyl-OR₃, —C₀-C₆ alkyl-C(O)—OR₃, —C₀-C₆ alkyl-C(O)NR₃, R₃ₐ, —CH═CH—C(O)—OR₃, —CH═CH—C(O)—N(R₃)(R₃ₐ), —N(R₃)—C(O)—CF₃, —N(R₃)—C₂-C₆ alkyl-N(R₃)(R₃ₐ), —C₀-C₆ alkyl-N(R₃)(R₃ₐ), —N(R₃)—C(O)—C₁-C₆ alkyl-R₃, —N(R₃)—S(O)₂—C₁-C₆ alkyl-R₃, —S(O)₂—N(R₃)R₃ₐ, —O—C₂-C₆ alkyl-N(R₃)(R₃ₐ), —S—R₃, —S(O)—C₁-C₆ alkyl-R₃, —S(O)₂—C₁-C₆ alkyl-R₃, C₃-C₆ cycloalkyl, heterocyclyl, C₄-C₇heterocyclyl-R₃, —O—C₂-C₄alkyl-heterocyclyl, —O-heterocyclyl-C(O)—OR₃, —NR₃—C₂-C₄alkyl-heterocyclyl, halo, —CF₃, —SO₃H, —CN, —C₁-C₆ alkylaryl, aryl, heteroaryl, —C₁-C₆ alkylheteroaryl, wherein each alkyl, alkenyl, alkynyl, cycloalkyl, heterocyclyl, aryl and heteroaryl moeity of the aformentioned R₄ and R₄ₐ are optionally substituted;
Z is selected from the group consisting of C₁-C₈ alkyl, C₁-C₈ alkenyl, C₁-C₈ alkynyl, C₁-C₈ heteroalkyl, —C₀-C₃alkyl-alkenyl-C₀-C₃-alkyl, —C₀-C₃alkyl-alkynyl-C₀-C₃-alkyl, —C₀-C₃alkyl-heteroalkyl-C₀-C₃-alkyl, aryl, —C₁-C₆ alkylaryl-, —C₀-C₆ alkylaryl-C₀-C₆-alkyl-, —C₀-C₆ alkylaryl-C₂-C₆-heteroalkyl-, —C₂-C₆ heteroalkylaryl-C₀-C₆-alkyl-, —C₄-C₆ heterocyclylaryl-C₀-C₆-alkyl-, —C₀-C₆ alkylaryl-C₄-C₆-heterocyclyl-, —C₀-C₆ alkylheteroaryl-, —C₀-C₆-alkylheteroaryl-C₀-C₆-alkyl-, heteroaryl, —C₀-C₆ alkylheteroaryl-C₂-C₆-heteroalkyl-, —C₂-C₆ heteroalkyl-heteroaryl-C₀-C₆-alkyl-, —C₄-C₆ heterocyclyl-heteroaryl-C₀-C₆-alkyl-, —C₀-C₆ alkyl-heteroaryl-C₄-C₆-heterocyclyl-, —C₃-C₆ alkynyl-aryl-C₀-C₆-alkyl, —C₀-C₆ alkyl-aryl-C₃-C₆-alkynyl, —C₃-C₆ alkynyl-heteroaryl-C₀-C₆-alkyl, —C₀-C₆ alkyl-heteroaryl-C₃-C₆-alkynyl, —C₃-C₆ alkenyl-aryl-C₀-C₆-alkyl, —C₀-C₆ alkyl-aryl-C₃-C₆-alkenyl, —C₃-C₆ alkenyl-heteroaryl-C₀-C₆-alkyl, —C₀-C₆ alkyl-heteroaryl-C₃-C₆-alkenyl, —C₀-C₆ alkylaryl-aryl-, —C₀-C₆ alkylaryl-aryl-C₀-C₆-alkyl-, —C₀-C₆ alkylaryl-heteroaryl-, —C₀-C₆ alkyl-aryl-heteroaryl-C₀-C₆-alkyl-, —C₀-C₆ alkyl-C₃-C₆ cycloalkyl-, —C₀-C₆ alkyl-C₃-C₆ cycloalkyl-C₀-C₆-alkyl-, —C₁-C₆ alkyl-X—C₃-C₆ cycloalkyl-, —C₁-C₆ alkyl-X—C₃-C₆ cycloalkyl-C₀-C₆-alkyl-, —C₁-C₆ alkyl-N(R₃)—C₁-C₆ alkyl-C₃-C₆ cycloalkyl-, —C₁-C₆ alkyl-N(R₃)—C₁-C₆ alkyl-C₃-C₆ cycloalkyl-C₀-C₆-alkyl-, and —C₁-C₆ alkyl-S—S—C₁-C₆ alkyl-, wherein each alkyl, alkenyl, alkynyl, heteroalkyl, aryl, heteroaryl, heterocyclyl, and cycloalkyl moiety is optionally substituted;
L is selected from the group consisting of a covalent bond, —C₀-C₆alkyl-aryl-C₀-C₃alkyl-X—C₀-C₃alkyl, C₀-C₆alkyl-heteroaryl-C₀-C₃alkyl-X—C₀-C₃alkyl, C₀-C₃alkyl-X—C₀-C₃alkyl, C₀-C₆alkyl-N(R₃)—C(O)—N(R₃)—S(O)₂—C₀-C₃alkyl-aryl, —C₀-C₃alkyl-S(O)₂—N(R₃)—C₀-C₃alkyl-aryl-C₀-C₃alkyl-C(O)—N(R₃)—C₀-C₃alkyl, —C₀-C₃alkyl-N(R₃)—C(O)—O-heterocyclyl-C₀-C₃alkyl, C₀-C₃alkyl-heteroaryl-C₀-C₃alkyl-N(R₃)—C₀-C₃alkyl, —C₀-C₆ alkyl-N(R₃)C(O)heterocyclyl-C₀-C₃alkyl, —C₀-C₆ alkyl-S(O)₂heterocyclyl-C₀-C₃alkyl-, —C₀-C₆ alkyl-N(R₃)S(O)₂heterocyclyl-C₀-C₃alkyl, —C₀-C₆ alkyl-, —C₂-C₆ alkenyl-, —C₂-C₆ alkynyl-, —C₀-C₆ alkyl-N(R₃)C(O)—C₀-C₃ alkyl, —C₀-C₆ alkyl-N(R₃)C(S)—C₀-C₃ alkyl, —C₀-C₆ alkyl-C(O)N(R₃)C(O)—C₀-C₃ alkyl, —C₂-C₆ heteroalkyl-N(R₃)C(O)—C₀-C₃ alkyl, —C₀-C₆ alkyl-C(O)—N(R₃)—C₀-C₃ alkyl, —C₀-C₆ alkyl-C(S)—N(R₃)—C₀-C₃ alkyl, —C₀-C₆ alkyl-OC(O)—, —C₀-C₆ alkyl-C(O)—O—, —C₀-C₆ alkyl-C(O)—C₀-C₃ alkyl, —C₀-C₆ alkyl-SO₂—N(R₃)—C₀-C₃ alkyl, —C₀-C₆ alkyl-N(R₃)—SO₂—C₀-C₃ alkyl, —C₀-C₆ alkyl-C(O)—N(R₃)—SO₂—C₀-C₃ alkyl, —C₀-C₆ alkyl-C(O)—N(R₃)—SO₂—C₀-C₃ alkyl-aryl, —C₀-C₆ alkyl-C(O)—N(R₃)—SO₂—C₀-C₃ alkyl-heteroaryl, —C₀-C₆ alkyl-N(R₃)—C₀-C₃ alkyl, —C₀-C₆ alkyl-N(R₇)—C₀-C₃ alkyl, —C₀-C₆ alkyl-S—C₀-C₃ alkyl, —C₀-C₆ alkyl-O—C₀-C₃ alkyl-, —C₀-C₆ alkyl-S(O)—C₀-C₃ alkyl, —C₀-C₆ alkyl-S(O)₂—C₀-C₃ alkyl, —C₀-C₆ alkyl-(CR₃═CR₃)₁₋₂—C₁-C₆ alkyl-, —C₀-C₆ alkyl-(C≡C)₁₋₂—C₁-C₆ alkyl-, —C₀-C₆ alkyl-N(R₃)—C(O)—N(R₃)—C₀-C₃ alkyl, —C₀-C₆ alkyl-N(R₃)—C(O)—O—C₀-C₃ alkyl, —C₀-C₆ alkyl-O—C(O)—N(R₃)—C₀-C₃ alkyl, —C₀-C₃ alkyl N(R₃)C(O)—C₀-C₃ alkyl-heterocyclyl-C(O)—C₀-C₆ alkyl, —C₀-C₃ alkyl-heterocyclyl-C₀-C₃ alkyl, —C₀-C₃ alkyl-heterocyclyl-C₂-C₄ alkenyl-C₀-C₃ alkyl, —C₀-C₃ alkyl-heterocyclyl-C₀-C₃ alkyl-O-C₀-C₃ alkyl, —C₀-C₃ alkyl-O—C₀-C₃ alkyl heterocyclyl-C₀-C₃ alkyl, —C₀-C₃ alkyl-heterocyclyl-C₀-C₃ alkyl-N(R₃)—C₀-C₃ alkyl, —C₀-C₃ alkyl-N(R₃)—C₀-C₃ alkyl heterocyclyl-C₀-C₃ alkyl, —C₀-C₃ alkyl-heterocyclyl-C₀-C₃ alkyl-S—, —C₀-C₃ alkyl S(O)₂N(R₃)—C₀-C₃ alkyl heterocyclyl-C₀-C₃ alkyl, —C₀-C₃ alkyl S(O)₂-heterocyclyl-C₀-C₃ alkyl, —C₀-C₃ alkyl-C(O)N(R₃)—C₀-C₃ alkyl-heterocyclyl-C₀-C₃ alkyl, —C₀-C₃ alkyl-C(S)N(R₃)—C₀-C₃ alkyl-heterocyclyl-C₀-C₃ alkyl, —C₀-C₃ alkyl C(O)-heterocyclyl-C₀-C₃ alkyl, —C₀-C₃ alkyl OC(O)N(R₃)—C₀-C₃ alkyl-heterocyclyl-C₀-C₃ alkyl, —C₀-C₃ alkyl-OC(S)N(R₃)—C₀-C₃ alkyl-heterocyclyl-C₀-C₃ alkyl, —C₀-C₃ alkyl-OC(O)-heterocyclyl-C₀-C₃ alkyl, —C₀-C₃ alkyl N(R₃)C(O)N(R₃)—C₀-C₃ alkyl-heterocyclyl-C₀-C₃ alkyl, —C₀-C₃ alkyl N(R₃)C(S)N(R₃)—C₀-C₃ alkyl-heterocyclyl-C₀-C₃ alkyl, —C₀-C₃ alkyl N(R₃)C(O)-heterocyclyl-C₀-C₃alkyl, —C₀-C₃ alkyl N(R₃)C(S)-heterocyclyl-C₀-C₃ alkyl, —C₀-C₃ alkyl N(R₃)S(O)₂N(R₃)—C₀-C₃ alkyl-heterocyclyl-C₀-C₃ alkyl, —C₀-C₃ alkyl-C═N—O—C₀-C₃ alkyl, —C₀-C₃ alkyl N(R₃)C(O)—C₁-C₃ alkyl-N(R₃)C(O)—C₀-C₃ alkyl, —C₀-C₃ alkyl N(R₃)C(S)—C₁-C₃ alkyl-N(R₃)C(S)—C₀-C₃ alkyl, —S(O)₂—N(R₃)—C₀-C₃ alkyl-aryl-C₀-C₃ alkyl-N(R₃)C(O)—C₁-C₃ alkyl-, —S(O)₂—N(R₃)—C₀-C₃ alkyl-aryl-C₀-C₃ alkyl-N(R₃)C(S)—C₁-C₃ alkyl-, —N(R₃)—S(O)₂—C₀-C₃ alkyl-aryl-C₀-C₃ alkyl-N(R₃)C(O)—C₁-C₃ alkyl-, —N(R₃)—S(O)₂—C₀-C₃ alkyl-aryl-C₀-C₃ alkyl-N(R₃)C(S)—C₁-C₃ alkyl-, —S(O)₂—N(R₃)—C₀-C₃ alkyl-heteroaryl-C₀-C₃ alkyl-N(R₃)C(O)—C₁-C₃ alkyl-, —S(O)₂—N(R₃)—C₀-C₃ alkyl-heteroaryl-C₀-C₃ alkyl-N(R₃)C(S)—C₁-C₃ alkyl-, —N(R₃)—S(O)₂—C₀-C₃ alkyl-heteroaryl-C₀-C₃ alkyl-N(R₃)C(O)—C₁-C₃ alkyl- and —N(R₃)—S(O)₂—C₀-C₃ alkyl-heteroaryl-C₀-C₃ alkyl-N(R₃)C(S)—C₁-C₃ alkyl-, wherein each alkyl, alkenyl, alkynyl, heteroalkyl, cycloalkyl, heterocycloalkyl, aryl and heteroaryl moiety of the aforementioned L are optionally substituted,
—(C₀-C₃alkyl)(R₃)N—S(O)₂—N(R₃)—C₂-C₄alkyl-O—C₀-C₃alkyl, when Y is absent,
—R₃R₃ₐNS(O)₂N(R₃)—C₂-C₄alkyl-O—C₀-C₆ alkyl-, when Y is absent, and
—R₃R₃ₐNS(O)₂N(R₃)—C₂-C₄alkyl, when Y is absent,
wherein the right end-attaches to Z and the left end attaches to Y;
Y is selected from the group consisting of alkyl, heteroalkyl, cycloalkyl, heterocyclyl, alkylcycloalkyl, alkylheterocyclyl, aryl, alkylaryl, heteroaryl, alkylheteroaryl, aryl-heteroaryl, alkylaryl-heteroaryl, heteroaryl-alkylaryl, aryl-aryl, alkylaryl-aryl, aryl-alkylaryl, heteroaryl-heteroaryl, heteroaryl-aryl, alkylheteroaryl-aryl, aryl-alkylheteroaryl, heteroaryl-aryl-aryl, aryl-aryl-heteroaryl, alkylheteroaryl-aryl-aryl, aryl-aryl-alkylheteroaryl, heteroaryl-aryl-heteroaryl, alkylheteroaryl-aryl-heteroaryl, heteroaryl-aryl-alkylheteroaryl, alkylheteroaryl-heteroaryl, heteroaryl-alkylheteroaryl, heterocyclyl-heteroaryl, heteroaryl-heterocyclyl, heterocyclyl-aryl, aryl-heterocyclyl, heterocyclyl-alyl-aryl, aryl-alkyl-heterocyclyl, aryl-C₁-C₃alkyl-aryl, —(O)C—C₀-C₃alkyl-aryl, C₀-C₃alkyl-aryl, —C₀-C₃alkyl-aryl-O—C₂-C₄alkyl-N(R₃)(R₃ₐ), —C₀-C₃alkyl-heteroaryl-O—C₂-C₄alkyl-N(R₃)(R₃ₐ), —C₀-C₃alkyl-aryl-C₀-C₃alkyl, C₀-C₃alkyl-heteroaryl-C₀-C₃alkyl and aryl-C₁-C₃alkyl-heteroaryl, each optionally substituted with one or more groups selected from R₃, R₄ or R₇; or
Y-L-Z- is selected from the group consisting of aryl-C₂-C₆ alkynyl-C₁-C₄alkyl, heteroaryl-C₂-C₆-alkynyl-C₁-C₄alkyl, R₃-heterocyclyl-C₀-C₃ alkyl-NR₃C(O)NR₃-heteroaryl-C₂-C₇alkyl; R₃-heterocyclyl-C₀-C₃ alkyl-NR₃C(O)NR₃-aryl-C₂-C₇alkyl; aryl-C₀-C₆ alkyl-, heteroaryl-C₁-C₆ alkyl-N(R₄)—C₁-C₆-alkyl-aryl-C₀-C₆ alkyl-, heteroaryl-C₀-C₆ alkyl-heteroaryl-C₀-C₇ alkyl-aryl-C₀-C₆-alkyl, heteroaryl-C₀-C₆ alkyl-, aryl-C₀-C₆ alkyl-heteroaryl-C₀-C₆ alkyl-, aryl-C₀-C₆ alkyl-aryl-C₀-C₆ alkyl-, aryl-C₀-C₆ alkyl-aryl-C₀-C₆ alkenyl-, aryl-C₀-C₆ alkyl-aryl-C₀-C₆ alkynyl-, aryl-C₀-C₆ alkenyl-aryl-C₀-C₆ alkyl-, aryl-C₀-C₆ alkenyl-aryl-C₀-C₆ alkenyl-, aryl-C₀-C₆ alkenyl-aryl-C₀-C₆ alkynyl-, aryl-C₀-C₆ alkynyl-aryl-C₀-C₆ alkyl-, aryl-C₀-C₆ alkynyl-aryl-C₀-C₆ alkenyl-, aryl-C₀-C₆ alkynyl-aryl-C₀-C₆ alkynyl-, heteroaryl-C₀-C₆ alkyl-aryl-C₀-C₆ alkyl-, heteroaryl-C₀-C₆ alkyl-aryl-C₀-C₆ alkenyl-, heteroaryl-C₀-C₆ alkyl-aryl-C₀-C₆ alkynyl-, heteroaryl-C₀-C₆ alkenyl-aryl-C₀-C₆ alkyl-, heteroaryl-C₀-C₆ alkenyl-aryl-C₀-C₆ alkenyl-, heteroaryl-C₀-C₆ alkenyl-aryl-C₀-C₆ alkynyl-, heteroaryl-C₀-C₆ alkynyl-aryl-C₀-C₆ alkyl-, heteroaryl-C₀-C₆ alkynyl-aryl-C₀-C₆ alkenyl-, heteroaryl-C₀-C₆ alkynyl-aryl-C₀-C₆ alkynyl-, aryl-C₀-C₆ alkyl-heteroaryl-C₀-C₆ alkyl-, aryl-C₀-C₆ alkyl-heteroaryl-C₀-C₆ alkenyl-, aryl-C₀-C₆ alkyl-heteroaryl-C₀-C₆ alkynyl-, aryl-C₀-C₆ alkenyl-heteroaryl-C₀-C₆ alkyl-, aryl-C₀-C₆ alkenyl-heteroaryl-C₀-C₆ alkenyl-, aryl-C₀-C₆ alkenyl-heteroaryl-C₀-C₆ alkynyl-, aryl-C₀-C₆ alkynyl-heteroaryl-C₀-C₆ alkyl-, aryl-C₀-C₆ alkynyl-heteroaryl-C₀-C₆ alkenyl-, aryl-C₀-C₆ alkynyl-heteroaryl-C₀-C₆ alkynyl-, heteroaryl-C₀-C₆ alkyl-heteroaryl-C₀-C₆ alkyl-, heteroaryl-C₀-C₆ alkyl-heteroaryl-C₀-C₆ alkenyl-, heteroaryl-C₀-C₆ alkyl-heteroaryl-C₀-C₆ alkynyl-, heteroaryl-C₀-C₆ alkenyl-heteroaryl-C₀-C₆ alkyl-, heteroaryl-C₀-C₆ alkenyl-heteroaryl-C₀-C₆ alkenyl-, heteroaryl-C₀-C₆ alkenyl-heteroaryl-C₀-C₆ alkynyl-, heteroaryl-C₀-C₆ alkynyl-heteroaryl-C₀-C₆ alkyl-, heteroaryl-C₀-C₆ alkynyl-heteroaryl-C₀-C₆ alkenyl-, heteroaryl-C₀-C₆ alkynyl-heteroaryl-C₀-C₆ alkynyl-, heteroaryl-C₀-C₆ alkyl-heteroaryl-C₀-C₇ alkyl-aryl-, heteroaryl-C₀-C₆ alkyl-heteroaryl-C₀-C₇ alkyl-aryl-C₀-C₃-alkyl, aryl-C₀-C₆ alkyl-heteroaryl-C₀-C₇ alkyl-aryl-, aryl-C₀-C₆ alkyl-heteroaryl-C₀-C₇ alkyl-aryl-C₀-C₃-alkyl, aryl-C₀-C₆ alkyl-heteroaryl-C₀-C₇ alkyl-heteroaryl-, aryl-C₀-C₆ alkyl-heteroaryl-C₀-C₇ alkyl-heteroaryl-C₀-C₃-alkyl, heteroaryl-C₀-C₆ alkyl-heteroaryl-C₀-C₇ alkyl-aryl-, heteroaryl-C₀-C₆ alkyl-heteroaryl-C₀-C₇ alkyl-aryl-C₀-C₃-alkyl, heteroaryl-C₀-C₆ alkyl-heteroaryl-C₀-C₇ alkyl-, heteroaryl-C₀-C₆ alkyl-aryl-C₀-C₇ alkyl-, heteroaryl-C(O)—C₀-C₆ alkyl-heteroaryl-C₀-C₇ alkyl-, heteroaryl-C(O)—C₀-C₆ alkyl-aryl-C₀-C₇ alkyl-, heteroaryl-S(O)₂—C₀-C₆ alkyl-heteroaryl-, heteroaryl-C₀-C₆ alkyl-N(R₃)—C(O)—C₁-C₇ alkyl-, aryl-C₀-C₆ alkyl-C(O)—N(R₃)—C₁-C₇ alkyl-, heteroaryl-C₀-C₆ alkyl-C(O)—N(R₃)—C₁-C₇ alkyl-, C₁-C₆ alkyl-C(O)—N(R₃)—C₁-C₇ alkyl-, heterocyclyl-C₀-C₆ alkyl-C(O)—N(R₃)—C₁-C₇ alkyl-, C₁-C₃ cycloalkyl-C(O)—N(R₃)—C₁-C₇ alkyl-, (R₃)(R₃ₐ)N—C₀-C₆alkyl-C(O)—N(R₃)—C₁-C₇alkyl, aryl-C₀-C₆alkyl-O—C(O)—N(R₃)—C₁-C₇alkyl, aryl-C₁-C₃ alkyl-N(R₃)—C(O)—C₁-C₇ alkyl-, C₁-C₃ alkyl-N(R₃)—C(O)—C₁-C₇ alkyl-, heteroaryl-S(O)₂—C₀-C₆ alkyl-aryl-, aryl-C₀-C₆ alkyl-N(R₃)—C(O)—C₁-C₇ alkyl-, —R₃—O—C(O)NR₃—C₀-C₃alkyl-heteroaryl-C₁-C₇alkyl-, R₃—C(O)—C₀-C₃alkyl-heteroaryl-C₁-C₇alkyl-, R₃—C(O)-heterocyclyl-C₀-C₃alkyl-heteroaryl-C₀-C₇alkyl-, R₃-heterocyclyl-C₀-C₃alkyl-N(R₃)C(O)N(R₃)—C₀-C₃alkyl-heteroaryl-C₀-C₇alkyl-, R₃-heterocyclyl-C₀-C₃alkyl-N(R₃)C(O)—C₀-C₃alkyl-heteroaryl-C₀-C₇alkyl- and R₃-heterocyclyl-C₀-C₃alkyl-N(R₃)S(O)₂—C₀-C₃alkyl-heteroaryl-C₀-C₇alkyl-, wherein the alkyl, alkenyl, alkynyl, heteroalkyl, cycloalkyl, aryl, heterocyclyl and heteroaryl moieties of the aforementioned Y-L-Z are optionally substituted,
Y—C₁-C₃ alkyl-N(R₃)—C(O)—C₁-C₇ alkyl- wherein the C₁-C₃ alkyl is optionally substituted with —C(O)NR₃—C₁-C₃ alkyl A₁ₐ and C₁-C₇ alkyl is optionally substituted with —NR₃—C(O)O-C₁-C₃ alkyl-A_{1b}, —NR₃—C(O)—C₁-C₃ alkyl-A_{1b}, —NR₃—S(O)₂—C₁-C₃ alkyl-A_{1b}, —NR₃—C(O)—NR₃—C₁-C₃ alkyl-A_{1b} or —NR₃—S(O)₂—NR₃-C₁-C₃ alkyl-A_{1b}, aryl-C₁-C₃ alkyl-N(R₃)—C(O)—C₁-C₇ alkyl- wherein the C₁-C₃ alkyl is optionally substituted with —C(O)NR₃—C₁-C₃ alkyl-A₁ₐ and C₁-C₇ alkyl is optionally substituted with —NR₃—C(O)O—C₁-C₃ alkyl-A_{1b}, —NR₃—C(O)—C₁-C₃ alkyl-A_{1b}, —NR₃—S(O)₂—C₁-C₃ alkyl-A_{1b}, —NR₃—C(O)—NR₃—C₁-C₃ alkyl A_{1b} or —NR₃—S(O)₂—NR₃-C₁-C₃ alkyl-A_{1b}, A₂ₐ-arylene-C₀-C₃ alkyl-N(R₃)—C(O)—C₁-C₇ alkyl-, wherein the C₁-C₇ alkyl is optionally substituted with —NR₃—C(O)—C₁-C₅ alkyl-C₂-C₄ alkenyl-C₁-C₃ alkyl-O-A_{2b}, or —N(R₃)—C(O)—C₁-C₇alkyl-O-A_{2b},
A₂ₐ-heteroarylene-C₀-C₃ alkyl-N(R₃)—C(O)—C₁-C₇ alkyl-, wherein the C₁-C₇ alkyl is optionally substituted with —NR₃—C(O)—C₁-C₅ alkyl-C₂-C₄ alkenyl-C₁-C₃ alkyl-O-A_{2b},
A₁ₐ-O-aryl-C₀-C₃alkyl-N(R₃)—C(O)—C₁-C₇alkyl, wherein the C₁-C₇ alkyl is optionally substituted with —N(R₃)—C(O)—C₁-C₇alkyl-A_{1b}, and
B₂—B₁—N(R₃)—C(O)—C₁-C₇ alkyl-, wherein the C₁-C₇ alkyl is optionally substituted with —NR₃—B₃ and the amine of B₃ is conected with the acid of B₂ to form a amidepeptide bond.
heteroaryl-S(O)₂—C₀-C₆ alkyl-aryl- or aryl-C₀-C₆ alkyl-N(R₃)—C(O)—C₁-C₇ alkyl-, wherein each of the aryl and heteroaryl groups is optionally substituted with one or more groups selected from oxo, —NO₂, C₁-C₆ alkoxy, halo, R₃, R₄ or R₆; wherein or
A₁ₐ and A_{1b} are independently selected from the group consisting of alkyl, alkenyl and a, protecting group; or
A₁ₐ and A_{1b} together, they are attached to via a form a ring with —C₂-C₆alkylene, —C₂-C₆alkenylene or, —C₂-C₆alkynylene linker, form an optionally substituted ring; moieties.
A₂ₐ and A_{2b} together are a covalent bond and, together with the aryl to which they are attached to form a ring; and
B₁, B₂ and B₃ are attached to form peptide bond.
B₁, B₂ and B₃ are each independently D or L-Gly, D or L-Pro, D or L-Tyr, D or L-Tyr(OR₃), D or L-Phe, D or L-PheR₄, D or L-A_{1b}, D or L-Ala, D or L-ProR₃, D or L-Ile, D or L-Leu. D or L-PheR₃, D or L-Pip, a natural or synthetic amino acid; or
L is a covalent bond and Z is C₀-C₆alkyl, heteroalkyl, —C₀-C₆ alkyl-heterocyclyl-C₀-C₆ alkyl-, -heterocyclyl-C(O)—C₂-C₆ alkenyl-C₁-C₃ alkyl-, —C₀-C₇ alkyl-N(R₃)—C(O)-heterocyclyl-C₀-C₇ alkyl-, —C₀-C₇ alkyl-N(R₃)—C(S)-heterocyclyl-C₀-C₇ alkyl-, —C₀-C₇ alkyl-, O—C(O)-heterocyclyl-C₀-C₆ alkyl-, —C₀-C₇ alkyl-O—C(S)-heterocyclyl-C₀-C₆ alkyl-, —C₀-C₆ alkyl-C(O)-heterocyclyl-C₀-C₆ alkyl-, —C₀-C₆ alkyl-C(S)-heterocyclyl-C₀-C₆ alkyl-, —C₀-C₆ alkyl-S(O)₂-heterocyclyl-C₀-C₆ alkyl-, —C₀-C₆ alkyl-heterocyclyl-C(O)—C₀-C₆ alkyl-, —C₀-C₆ alkyl-heterocyclyl-C(S)—C₀-C₆ alkyl-, —C₀-C₆ alkyl-heterocyclyl-S(O)₂—C₀-C₆ alkyl-, —C₀-C₆ alkyl-heterocyclyl-N(R₃)C(O)—C₀-C₆ alkyl-, —C₀-C₆ alkyl-heterocyclyl-O—C(O)—C₀-C₆ alkyl-, —C₀-C₆ alkyl-heterocyclyl-N(R₃)C(S)—C₀-C₆ alkyl-, —C₀-C₆ alkyl-heterocyclyl-O—C(S)—C₀-C₆ alkyl-, and —X—C₁-C₆ alkyl-C(R₃)═C(R₃)—C₁-C₆ alkyl-, wherein each alkyl, alkenyl and heterocyclyl of the aforementioned Z is optionally substituted;
R₆ is selected from the group consisting of —H, C₁-C₆ alkyl, C₁-C₆ alkenyl, C₁-C₆ alkynyl, C₁-C₆ heteroalkyl, heterocyclyl-C₀-C₆ alkyl-, aryl-C₀-C₆ alkyl-, heteroaryl-C₀-C₆ alkyl-, C₃-C₆ cycloalkyl-C₀-C₆ alkyl-, N(R₃)(R₃ₐ)—C₁-C₆ alkyl- and N(R₃)(R₃ₐ)—C(O)—C₁-C₆ alkyl-, wherein each alkyl, alkenyl, alkynyl, heteoralkyl, cycloalkyl, aryl, heteroaryl, or heterocyclyl moiety is optionally substituted; and
R₇ and R₇ₐ are independently selected from the group consisting of —H, C₁-C₆ alkyl-, C₁-C₆ alkenyl, C₁-C₆ alkynyl, C₁-C₆ heteroalkyl, R₃—O—C₁-C₆ alkyl-, N(R₃)(R₃ₐ)—C₁-C₆ alkyl-, a protecting group, —C(O)—O—C₁-C₆ alkyl, —C(O)—O-benzyl and heterocyclyl-C₁-C₆ alkyl-, wherein each alkyl, alkenyl, alkynyl, heteroalkyl, benzyl and heterocyclyl moiety is independently optionally substituted; or
R₇ is —OR₃ when attached to the N atom of an indolyl moiety;
wherein in a —N(R₃)(R₃ₐ) group, optionally the R₃ and R₃ₐ together with the nitrogen atom to which they are attached form a heterocyclyl group; or
wherein in a —N(R₄)(R₄ₐ) group, optionally the R₄ and R₄ₐ together with the nitrogen atom to which they are attached form a heterocyclyl group; and
   provided that
when R₃, R₃ₐ, R₄ and R₄ₐ are present in —N(R₃)(R₃ₐ), —N(R₄) (R₄ₐ), —NR₃, —OR₃, —SR₃, -alkyl-R₃, —NR₃S(O)₂R₃, —S(O)CH₂R₃, —NR₃S(O)₂CH₂R₃, —NR₃C(O)CH₂R₃(C═NR₃)N(R₃)(R₃ₐ), —C(O)R₃, —NR₄ and —CR₃═CR₃, then R₃, R₃ₐ, R₄ and R₄ₐ are independently H, —C₁-C₆-alkyl, —C₃-C₆-cycloalkyl, heteroalkyl, aryl, alkyl-aryl, heteroaryl or alkyl-heteroary;
when Y-L-Z- is phenyl, W is nitrogen, X is a covalent bond or —CH₂—, R₁ and R₂ are —H, and Q is —C₁-C₆-alkyl-C(O)—OR₃, then R₃ is not —H;
when Y-L-Z- is phenyl, W is nitrogen, X is a covalent bond or —CH₂—, R₁ and R₂ are —H, and Q is —C₁-C₆-alkyl-N(R₃)(R₃ₐ), and R₃ is —H, then R₃ₐ is not —H;
when Y-L-Z- is phenyl, W is nitrogen, X is a covalent bond or —CH₂—, R₁ and R₂ are —H, Q is —C₁-C₆ alkyl-C(O)—N(R₃)(R₃ₐ), and R₃ is —H, then R₃ₐ is not —H, —OH, or phenyl substituted with —NH₂ or —OH;
when Y-L-Z- is phenyl or phenyl-CH₂—, W is nitrogen, X is a covalent bond or —CH₂—, R₁ and R₂ are —H, and Q is —C₁-C₆ alkyl-N(H)—S(O)₂—R₃, then R₃ is not —CH₃;
when Y-L-Z- is aryl-C₁-C₆ alkyl- or heteroaryl-C₁-C₆ alkyl-, W is nitrogen, X is a covalent bond or —CH₂—, R₁ and R₂ are —H, Q is —C₁-C₆alkyl-C(O)—N(R₃)(R₃ₐ), and R₃ is —H, then R₃ₐ is not —OH;
when Y-L-Z- is aryl-C₁-C₆ alkyl-, aryl, cycloalkyl, heterocyclyl, heteroaryl, or heteroaryl-C₁-C₆ alkyl-, W is nitrogen, X is a covalent bond or —CH₂—, R₁, R₂ and R₃ are —H, Q is not —C₁-C₆ alkyl-N(H)—C(O)—CH₂—SH;
when Y-L-Z- is aryl-C₁-C₆ alkyl-, aryl, or heteroaryl, W is nitrogen, R₁, R₂ and R₃ are —H, —X-Q is not —C₁-C₆alkyl-SH;
when Y-L-Z- is phenyl optionally para substituted with —N(CH₃)₂, naphthyl, indolyl, or benzofuranyl, W is nitrogen, X is a covalent bond or —CH₂—, R₁, R₂ and R₃ are —H, then Q is not —C₁-C₆ alkyl-OH;
when Y-L-Z- is phenyl optionally para substituted with —N(CH₃)₂, quinolinyl, biphenyl or benzyl, W is nitrogen, X is a covalent bond or —CH₂—, R₁, R₂ and R₃ are —H, then Q is not —C₁-C₆ alkyl-N(H)—S(O)₂—CH₃, —C₁-C₆ alkyl-S(O)₂—N(H)—OH, —C₁-C₆ alkyl-N(H)—C(O)—NH₂, —C₁-C₆ alkyl-N(H)—C(O)—C₁-C₂ alkyl-SH, —C₁-C₆ alkyl-C(O)—N(H)—OH, —C₁-C₆ alkyl-C(O)—OH, or —C₁-C₆ alkyl-N(H)—C(O)—O-t-butyl;
when Y-L-Z- is phenyl optionally para substituted with —N(CH₃)₂, biphenyl, substituted pyrrolyl, or substituted pyrrolidinyl, W is nitrogen, X is a covalent bond or —CH₂—, R₁, R₂ and R₃ are —H, then Q is not —C₁-C₆ alkyl-C(O)—N(H)—OH;
when Y-L-Z- is phenyl, benzyl, or quinolinyl, W is nitrogen, X is a covalent bond or —CH₂—, R₁ is —H, R₂ is —N(H)—C(O)—O-t-butyl or —N(H)—C(O)—O-benzyl, then Q is not —C₁-C₆ alkyl-C(O)—N(H)—OH, —C₁-C₆ alkyl-imidazolyl, —C₁-C₆ alkyl-SO₂—NH₂, —C₁-C₆ alkyl-C(O)—N(H)-imidazolyl, —C₁-C₆ alkyl-C(O)—N(H)-thiazolyl, —C₁-C₆ alkyl-C(O)—N(H)-pyridinyl, —C₁-C₆ alkyl-C(O)—N(H)-anilinyl, —C₁-C₆ alkyl-NH₂, —C₁-C₆ alkyl-N(H)—S(O)₂—CH₃, or —C₁-C₆ alkyl-C(O)—O—R₃, wherein R₃ is —CH₃, -t-butyl, or —H;
when Y-L-Z- is phenyl, W is nitrogen, X is a covalent bond or —CH₂—, R₁, R₂ and R₃ are —H, then Q is not C₁-C₆-alkyl-NH—S(O)₂—CH₃, C₁-C₆-alkyl-S(O)₂—NH—OH, C₁-C₆-alkyl-NH—C(O)—NH₂, —C₁-C₆-alkyl-NH₂, —C₁-C₆-alkyl-NH—C(O)—C₁-C₂-alkyl-halo, —C₁-C₆-alkyl-NH—C(O)—C₁-C₂-alkyl-NH₂ or —C₁-C₆-alkyl-NH—C(O)—CH₂—OH; or
when Y is phenyl optionally para substituted with —N(CH₃)₂, L is —C(O)—NH—CH₂—, Z is phenyl-CH₂, W is N, R₁, R₂ and R₃ are —H, and X is a covalent bond, Q is not —SH; and
   further provided that Formula (I) excludes those compounds wherein
X is S;
Q is selected from the group consisting of H, methyl, ethyl, phenyl, benzyl and acetyl; and
Y-L-Z is selected from the group consisting of R^{a}—(CH₂)₄₋₆ and R^{b}—Ar—(CH₂)₁₋₂—, wherein
R^{a} is selected from the group consisting of R^{c}NR^{d}C(O)—, R^{c}NHC(O)NH—, R^{c}NHC(S)NH—, R^{c}SO₂NH— and R^{c}C(O)NH—;
R^{b} is selected from the group consisting of R^{c}NR^{d}C(O)(CH₂)₁₋₂—, R^{c}NHC(O)NH(CH₂)₁₋₂—, R^{c}NHC(S)NH(CH₂)₁₋₂, R^{c}SO₂NH(CH₂)₁₋₂— and R^{c}C(O)NH(CH₂)₁₋₂—;
R^{c} is selected from the group consisting of C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁— alkyl-heteroaryl, -aryl-OCH₂-aryl, -aryl-CH₂O-aryl, -aryl-carbonyl-aryl, -aryl-C(O)CH₃, -aryl-O-aryl, -aryl-O-heterocyclyl, -aryl-C₁₋₄alkyl, -aryl-O—C₂₋₃alkyl-N(CH₃)(CH₃), C₀₋₁ alkyl-heterocyclyl-C₀₋₁alkyl, C₀₋₁ alkyl-heteroaryl-C₀₋₁ alkyl, -heterocyclyl, -heterocyclyl-aryl, -heterocyclyl-heteroaryl, -aryl-heterocyclyl, -aryl-heteroaryl and —CH(aryl)(aryl), any of which is optionally substituted with one or more of R^{e} or R^{f};
R^{e} or R^{f} are C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl or heterocyclyl; and
R^{d} is C₀₋₁alkyl, or R^{c} and R^{d} taken together form a heterocyclic or carbocyclic ring, any of which is optionally substituted with one or more independent C₀₋₄alkyl, halogen, —OH, —SCH₃, OCH₃, —NH₂, aryl, or heterocyclyl substituents; and
Ar is aryl optionally substituted with one or more independent C₁₋₄alkyl, halogen, —OH, —SCH₃, —OCH₃, —NH₂, aryl or heterocyclyl substituents; and
   further provided that Formula (I) excludes those compounds wherein 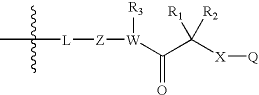
is —(CH₂)₃₋₄—NH(CO)—CH₂—O—CH₂-phenyl or —CH₂)₃₋₄—NHC(O)—CH₂—S-phenyl and
Y is selected from the group consisting of optionally substituted imidazopyridinyl or optionally substituted imidazonaphthyridine; and
   further provided that Formula (I) excludes those compounds wherein 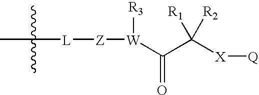
is —(CH₂)₂₋₃—NHC(O)—CH₂—O—CH₂-phenyl or —(CH₂)₂₋₃—NHC(O)—CH₂—S-phenyl, wherein the phenyl is optionally substituted with halogen, and Y is dimethyoxyphenyl; and
   further provided that Formula (I) excludes those compounds wherein 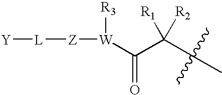
is 3-(R^{t})(R^{tt})CH-phenyl-1-O—C₃-C₄alkyl-NHC(O)—, 3-(R^{t})(R^{tt})CH-phenyl-1-O—C₃-C₄alkenyl-NHC(O)—, (R^{t})(R^{tt})CH-thiophene-O—C₃-C₄alkyl-NHC(O)—, (R^{t})(R^{tt})CH-thiophene-O—C₃-C₄alkenyl-NHC(O)—, (R^{t})(R^{tt})CH-pyridine-O—C₃-C₄alkyl-NHC(O)— and (R^{t})(R^{tt})CH-pyridinyl-O—C₃-C₄alkenyl-NHC(O)—, wherein R^{t} is selected from the group consisting of H, halogen, OH, Me, optionally substituted piperidino, dimethylamino, 1-pyrrolidinyl and 1-perhydroazepinyl, and R^{tt} is H or Me, or R^{t} is oxo and R^{tt} is absent, with the exception the this proviso does not include the compound 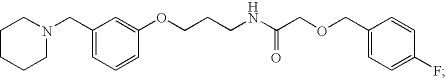
   and
further provided that Formula (I) excludes indol-(CH₂)₂—NHC(O)—CH₂—O—CH₂-phenyl, indol-(CH₂)₂—NHC(O)—CH₂—S(O)₂-phenyl-Me, phenyl-(CH₂)₂—NHC(O)—CH₂—S(O)₂-phenyl, phenyl-(CH₂)₂—NHC(O)—CH₂—S(O)₂-phenyl-Me, T-(CH₂)₂₋₅—NHC(O)—CH₂—S-phenyl (wherein T is pheny, fluro-phenyl, pyridine, methyl-pyrrolidine or methyl), NH₂—S(O)₂-phenyl-(CH₂)₂—NHC(O)—CH₂—S-phenyl, CH₃—(CH₂)₂—NHC(O)—CH₂—O—CH₂-phenyl, CH₃—(CH₂)₂—NHC(O)—CH₂—S(O)₂-phenyl, CH₃—(CH₂)₂—NHC(O)—CH₂—S-phenyl, N-[[[[(aryloxy- or -thio)alkyl]carbonyl]amino]alkyl]-2,2,5,5-tetramethyl-3-pyrroline-3-carboxamide, 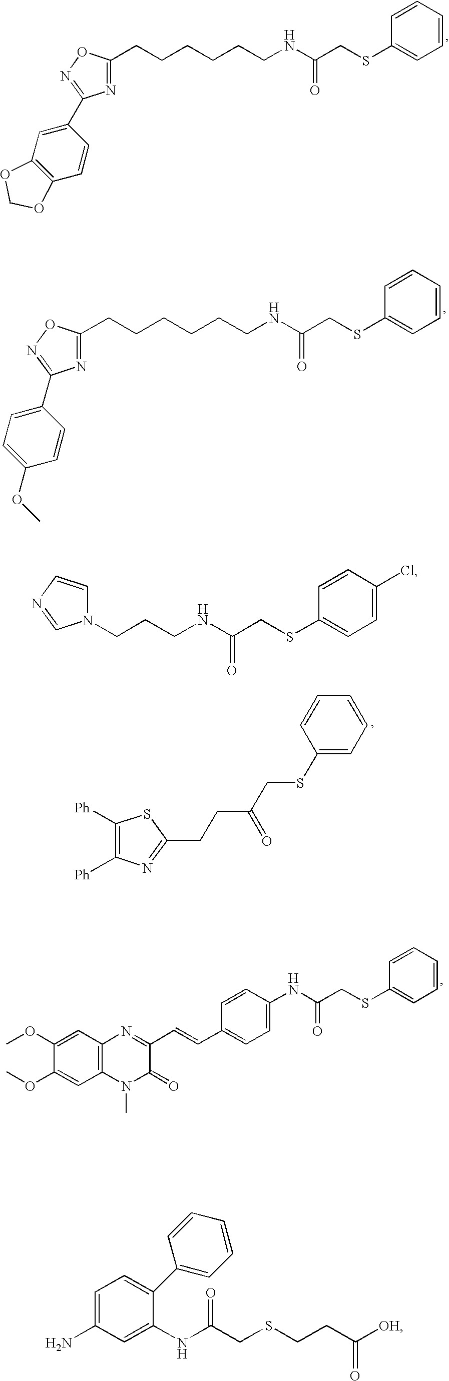 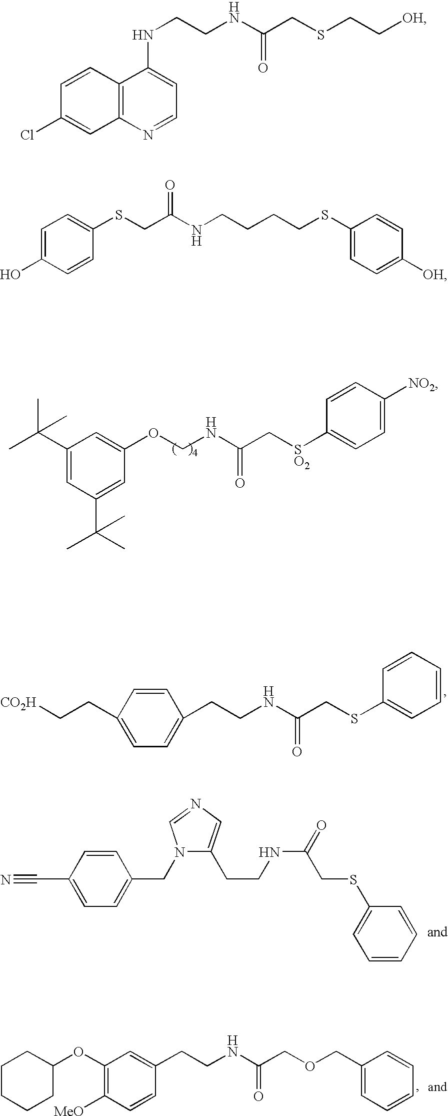
   and
further provided that Formula (I) excludes compounds of formula (R^{v})(R^{vv})pyrimidine-NHS(O)₂-phenyl-C₀-C₄alkyl-NHC(O)-A, wherein A is aryloxylalkyl or arylmercaptoalkyl, R^{v} is a lower alkyl, and R^{vv} is selected from the group consisting of H, unsubstituted or substituted alkyl, cycloalkyl, aryl, aralkyl, alkoxy, alkoxyalkyl and alkoxyalkoxy, or wherein R^{v} and R^{vv} taken together form a ring of 3 to 5 methylene groups which can contain oxygen or sulfur atoms.

A preferred embodiment, Embodiment A, provides compounds according to formula (I), wherein each alkyl, alkenyl, alkynyl, heteroalkyl, cycloalkyl, heterocyclyl, aryl and heteroaryl moiety of X-Q, Q, L, Z, R³ and R^{3a} is independently optionally substituted with one or more groups independently selected from R⁴.

A preferred embodiment, Embodiment B, provides compounds according to formula (I), wherein each alkyl, alkenyl, alkynyl, heteroalkyl, cycloalkyl, heterocyclyl, aryl and heteroaryl moiety of X-Q, Q, R³ and R^{3a} is independently optionally substituted with one or more groups independently selected from oxo, —OH, —CN, C₁-C₆ alkyl, C₁-C₆ alkoxy, —NO₂, —N(R₄)(R₄ₐ), halo, —SH, —S—C₁-C₆ alkyl, —S(O)—C₁-C₆ alkyl, —S—C(O)—C₁-C₆ alkyl and mono- to per-halogenated C₁-C₆ alkyl.

A preferred embodiment, Embodiment C, provides compounds according to formula (I), wherein C₁-C₆ alkyl of R₄ and R₄ₐ is optionally substituted with —OH, —NO₂ or C₀-C₆ alkyl-C(O)—N(R₃)(R₃ₐ).

A preferred embodiment, Embodiment D, provides compounds according to formula (I), wherein each alkyl, alkenyl, alkynyl, heteroalkyl, aryl, heteroaryl, cycloalkyl and heterocyclyl moiety of Z is independently optionally substituted with one or more groups independently selected from oxo, —OH, —CN, C₁-C₆ alkyl, C₁-C₆ alkoxy, —NO₂, —N(R₃)(R₃ₐ), halo, —SH and mono- to per-halogenated C₁-C₆ alkyl.

A preferred embodiment, Embodiment E, provides compounds according to formula (I), wherein L is selected from the group consisting of
—C₁-C₆ alkyl-N(R₃)—C₀-C₃ alkyl wherein the C₁-C₆ alkyl is optionally substituted with —C₁-C₄ alkyl-OR₃, or —C₀-C₄ alkyl-C(O)OR₃,
—C₀-C₆ alkyl-N(R₃)—C(O)—C₀-C₃ alkyl- wherein the C₁-C₆ alkyl is optionally substituted with —C₀-C₆ alkyl-O(R₃)—, —C₀-C₆ alkyl-C(O)O(R₃)— or —O—C₆ alkyl-N(R₃)(R₃ₐ)—,
—C₀-C₆ alkyl-N(R₃)—C(S)—C₀-C₃ alkyl- wherein the C₁-C₆ alkyl is optionally substituted with —C₀-C₆ alkyl-O(R₃)—, —C₀-C₆ alkyl-C(O)O(R₃)— or C₀-C₆ alkyl-N(R₃)(R₃ₐ)—,
—C₀-C₆ alkyl-C(O)—N(R₃)—C₀-C₃ alkyl- wherein the C₁-C₆ alkyl is optionally substituted with —C₀-C₆ alkyl-O(R₃)—, C₀-C₆ alkyl-C(O)O(R₃)— or C₀-C₆ alkyl-N(R₃)(R₃ₐ)—,
—C₀-C₆ alkyl-C(S)—N(R₃)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)—, and
—C₀-C₆ alkyl-N(R₃)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)—.

A preferred embodiment, Embodiment F, provides compounds according to formula (I), wherein each alkyl, alkenyl, alkynyl, heteroalkyl, aryl, heteroaryl, cycloalkyl and heterocyclyl moiety of Y-L-Z is independently optionally substituted with one or more groups independently selected from oxo, —NO₂, C₁-C₆ alkoxy, halo, R₃, R₄ and R₆.

A preferred embodiment, Embodiment G, provides compounds according to formula (I), wherein Y-L-Z- is selected from the group consisting of
heteroaryl-C₀-C₆ alkyl-N(R₃)—C(O)—C₁-C₇ alkyl-, wherein the C₁-C₇ alkyl is optionally substituted with —N(R₇)(R₇ₐ) or —N(R₃)—C(O)—C₁-C₆ alkyl-R₃,
aryl-C₀-C₆ alkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl or heteroaryl,
heteroaryl-C₀-C₆ alkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl or heteroaryl,
C₁-C₆ alkyl-C(O)—N(R₃)—C₁-C₇ alkyl-, wherein the C₁-C₇ alkyl is optionally substituted with —N(R₇)(R₇ₐ), aryl-aryl, heteroaryl-heteroaryl, heteroaryl-aryl or heteroaryl,
heterocyclyl-C₀-C₆ alkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl or heteroaryl,
C₁-C₆ cycloalkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl, or heteroaryl,
C₁-C₃ alkyl-N(R₃)—C(O)—C₁-C₇ alkyl-, wherein the C₁-C₃ alkyl is optionally substituted with —C(O)NR₃—C₁-C₃ alkyl-A₁ₐ and the C₁-C₇ alkyl is optionally substituted with —NR₃—C(O)O—C₁-C₃ alkyl-A_{1b}, —NR₃—C(O)—C₁-C₃ alkyl-A_{1b}, —NR₃—S(O)₂—C₁-C₃ alkyl-A_{1b}, —NR₃—C(O)—NR₃—C₁-C₃ alkyl-A_{1b} or —NR₃—S(O)₂—NR₃—C₁-C₃ alkyl-A_{1b}, and
aryl-C₁-C₃ alkyl-N(R₃)—C(O)—C₁-C₇ alkyl-, wherein the C₁-C₃ alkyl is optionally substituted with —C(O)NR₃—C₁-C₃ alkyl-A₁ₐ and the C₁-C₇ alkyl is optionally substituted with —N(R₃)—C(O)O—C₁-C₃ alkyl-A_{1b}, —N(R₃)—C(O)—C₁-C₃ alkyl-A_{1b}, —NR₃—S(O)₂—C₁-C₃ alkyl-A_{1b} —NR₃—C(O)—NR₃—C₁-C₃ alkyl A_{1b} or —NR₃—S(O)₂—NR₃-C₁-C₃ alkyl-A_{1b}.

A preferred embodiment, Embodiment H, provides compounds according to formula (I), wherein B₁, B₂ and B₃ are independently selected from the group consisting of D-Gly, L-Gly, D-Pro, L-Pro, D-Tyr, L-Tyr, D-Tyr(OR₃), L-Tyr(OR₃), D-Phe, L-Phe, D-PheR₄, L-PheR₄, D-Aib, L-Aib, D-Ala, L-Ala, D-ProR₃, L-ProR₃, D-Ile, L-Ile, D-Leu, L-Leu D-PheR₃, L-PheR₃, D-Pip and L-Pip.

A preferred embodiment, Embodiment I, provides compounds according to formula (I), wherein each alkyl, alkenyl and heterocyclyl moiety of Y-Z is independently optionally substituted with one or more groups independently selected from R⁴.

A preferred embodiment, Embodiment J, provides compounds according to formula (I), wherein each alkyl, alkenyl, alkynyl, heteoralkyl, cycloalkyl, aryl, heteroaryl, and heterocyclyl moiety of R₆ is independently optionally substituted with one or more groups independently selected from R₃ and R₄.

A preferred embodiment, Embodiment K, provides compounds according to formula (I), wherein each alkyl, alkenyl, alkynyl, heteroalkyl, benzyl and heterocyclyl moiety of R₇ and R₇ₐ is independently optionally substituted with one or more groups independently selected oxo, —OH, —CN, C₁-C₆ alkyl, C₁-C₆ alkoxy, —NO₂, —N(R₃)(R₃ₐ), halo, —SH and mono- to per-halogenated C₁-C₆ alkyl.

A preferred embodiment, Embodiment L, provides compounds according to formula (I), wherein Y is selected from the group consisting of aromatic polycycle, non-aromatic polycycle, mixed aryl and non-aryl polycycle, polyheteroaryl, non-aromatic polyheterocycle, mixed aryl and non-aryl polyheterocycle, each of which is optionally substituted.

A preferred embodiment, Embodiment M, provides compounds according to formula (I), wherein Y is selected from the group consisting of —(O)C—C₀-C₃alkyl-aryl, —C₀-C₃alkyl-aryl, —C₀-C₃alkyl-aryl-O—C₂-C₄alkyl-N(R₃)(R₃ₐ), —C₀-C₃alkyl-heteroaryl-O—C₂-C₄alkyl-N(R₃)(R₃ₐ), —C₀-C₃alkyl-aryl-C₀-C₃alkyl, C₀-C₃alkyl-heteroaryl-C₀-C₃alkyl and aryl-C₁-C₃alkyl-aryl, each of which is optionally substituted.

A preferred embodiment, Embodiment N, provides compounds according to formula (I), wherein Y is aryl-C₁-C₃alkyl-aryl, wherein C₁-C₃ alkyl is optionally substituted with C₀-C₃alkyl.

A preferred embodiment, Embodiment 0, provides compounds according to formula (I), wherein L is a covalent bond and Z is selected from the group consisting of
—C₀-C₇ alkyl-N(R₃)—C(O)-heterocyclyl-C₀-C₆ alkyl-, wherein the C₁-C₇ alkyl is optionally substituted with —C₀-C₃alkyl-C(O)OR₃ or —C₀-C₃alkyl-OR₃,
C₀-C₇ alkyl-O—C(O)-heterocyclyl-C₀-C₆ alkyl-, wherein the C₁-C₇ alkyl is optionally substituted with —C₀-C₃alkyl-C(O)OR₃ or —C₀-C₃alkyl-OR₃, and
—C₁-C₄alkyl-N(R₃)C(O)-heteorcyclyl-C₁-C₇alkyl, wherein the C₁-C₄alkyl is optionally substituted with C₀-C₃alkyl-C(O)OR₃ or C₀-C₃alkyl-OR₃.

A preferred embodiment, Embodiment P, provides compounds according to formula (I), wherein X is —S—, —SO—, —SO₂—, —O—, —NR₃—, —CH₂—, —CH(OH)—, or

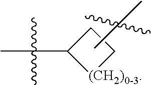

Yet another preferred embodiment, Embodiment Q, provides compounds according to formula (I), wherein R₁ and R₂ are independently —H, C₁-C₃ alkyl, C₃-C₆ cycloalkyl, aryl, or aryl-C₁-C₃ alkyl-. Preferably, R₁ and R₂ are independently —CH₃, —CH₂CH₃, phenyl or benzyl.

One other preferred embodiment, Embodiment R, provides compounds according to formula (I), wherein R₁ and R₂ together with the carbon atom to which they are attached form a 3- to 6-membered cycloalkyl or heterocyclyl group.

Another preferred embodiment, Embodiment S, provides compounds according to formula (I), wherein R₃ and R₃ₐ are independently —H, C₁-C₆ alkyl, C₃-C₆ cycloalkyl, —C₁-C₆ alkylaryl, aryl or heteroaryl. Preferably, R₃ and R₃ₐ are independently —C₁-C₆ alkylaryl, or aryl. More preferably, R₃ and R₃ₐ are independently phenyl or benzyl. Also preferred are compounds wherein R₃ and R₃ₐ are C₁-C₄ alkyl. Preferably, R₃ and R₃ₐ are independently t-butyl or i-propyl.

Another preferred embodiment, Embodiment T, provides compounds wherein in a NR₃R₃ₐ group or a NR₄R₄ₐ group, optionally the R₃ together or the R₄ together with the nitrogen atom to which they are attached form a group selected from morpholinyl, piperazinyl, piperidinyl, pyrrolydinyl, and azetidinyl.

Another preferred embodiment, Embodiment U, provides compounds according to formula (I), wherein X-Q is —OH, —NH₂, —Cl, —F, —SH or —Br. Preferably, X-Q is absent and R₁ and R₂ together with the carbon atom to which they attached form a 5- to 6-membered aromatic or heteroaromatic ring.

Embodiment V is another preferred embodiment according to formula (I), wherein Q is preferably one of the following groups:

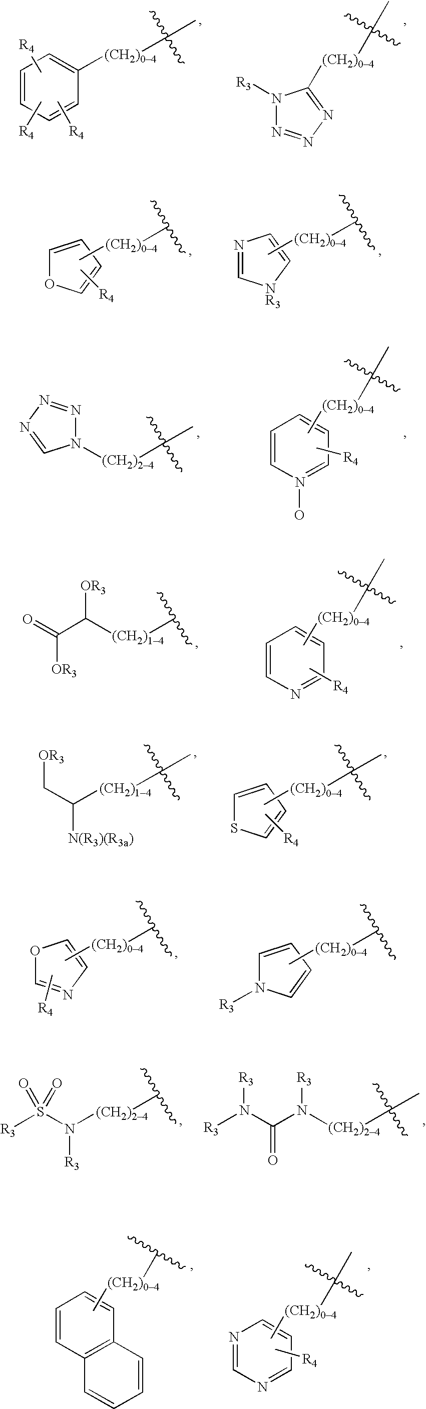

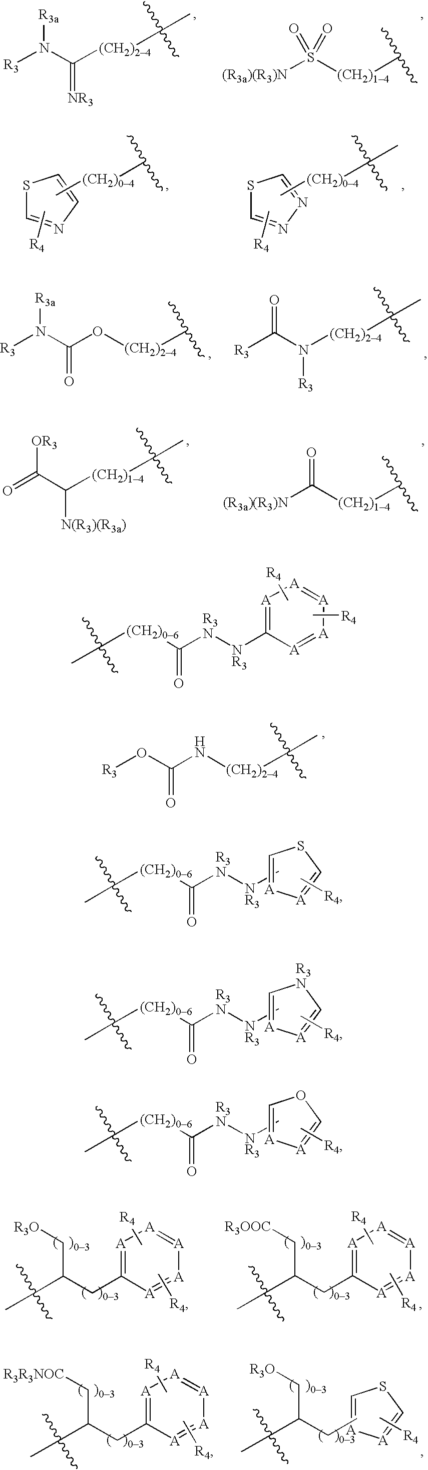

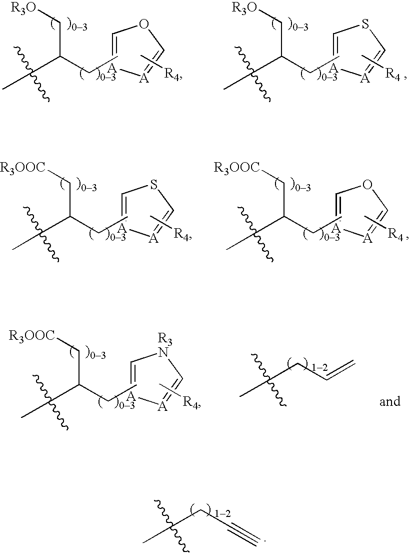

Another preferred embodiment according to formula (I), Embodiment W, provides compounds wherein R₄ is —H, —CH₃, —(CH₂)₀₋₄OR₃, —(CH₂)₀₋₄N(R₃)(R₃ₐ), —F, —Cl, —Br, —CF₃, —CN, —CH₂OH, —NO₂, —N(R₃)C(O)CH₂R₃, —N(R₃)SO₂CH₂R₃, —O(CH₂)₂₋₄N(R₃)(R₃ₐ), —SR₃, —S(O)CH₂R₃, —SO₂CH₂R₃, —(CH₂)₀₋₄C(O)OR₃, —CH═CHC(O)OR₃, —CH═CHC(O)N(R₃)R₃ₐ), —N(R₃)C(O)CF₃ or —N(R₃)(CH₂)₂N(R₃)(R₃ₐ).

Embodiment X is another preferred embodiment according to formula (I), wherein Z is preferably one of the following groups: 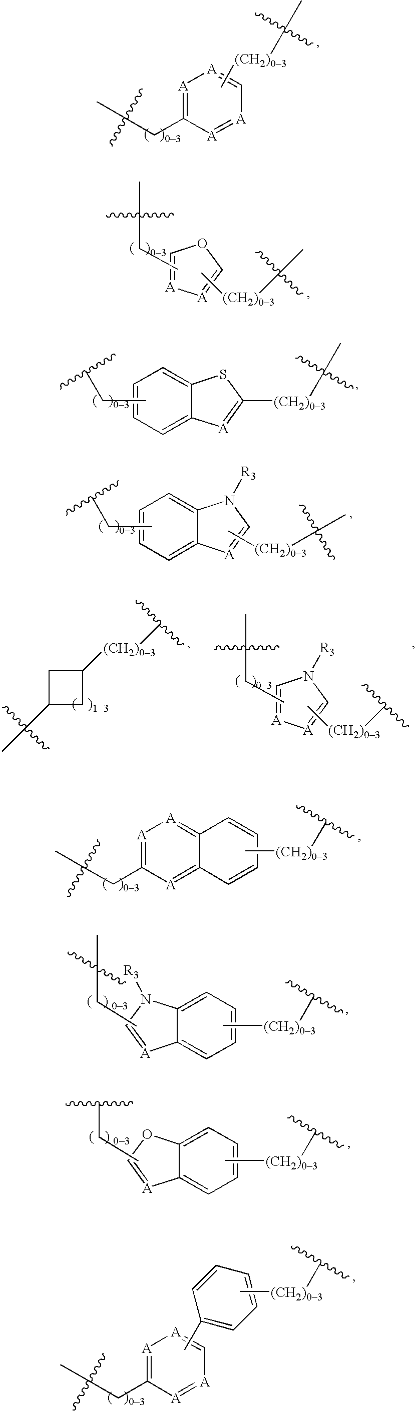 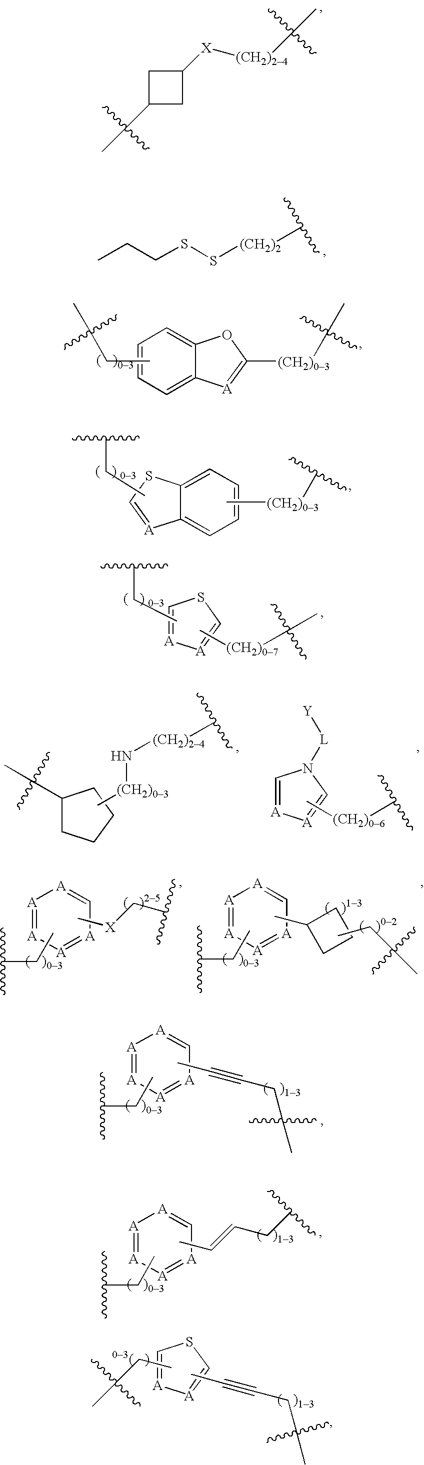 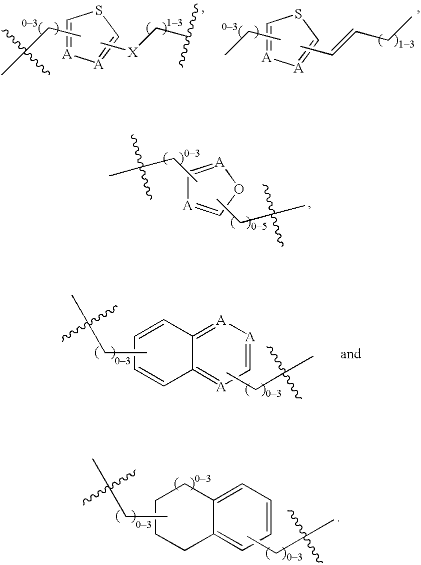
wherein A is —CH═ or —N═.

In a preferred Embodiment Y according to formula (I), the compounds are compounds wherein L is selected from the group consisting of a covalent bond, —(CH₂)₀₋₃ N(R₃)C(O)—, —(CH₂)₀₋₃C(O)N(R₃)—, —(CH₂)_{0-3O}C(O)—, —(CH₂)₀₋₃C(O)O—, —C₀-C₆ alkyl-N(R₃)C(O)heterocyclyl-C₀-C₃alkyl, —C₀-C₆ alkyl-S(O)₂heterocyclyl-C₀-C₃alkyl-, —(CH₂)₀₋₃C(O)—(CH₂)₀₋₃, —(CH₂)₀₋₃ SO₂N(R₃)—(CH₂)₀₋₃, —(CH₂)₀₋₃ NR₃S(O)₂—(CH₂)₀₋₃, —(CH₂)₀₋₃N(R₃)—(CH₂)₀₋₃, —(CH₂)₀₋₃N(R₇)—(CH₂)₀₋₃, —(CH₂)₀₋₃S—(CH₂)₀₋₃, —(CH₂)₀₋₃O—(CH₂)₀₋₃, —(CH₂)₀₋₃S(O)—(CH₂)₀₋₃, —(CH₂)₀₋₃S(O)₂—(CH₂)₀₋₃, —(CH₂)₀₋₃CH═CH—(CH₂)₂₋₃—, —(CH₂)₀₋₃N(R₃)C(O)N(R₃)—(CH₂)₀₋₃, —(CH₂)₀₋₃N(R₃)C(O)O—(CH₂)₀₋₃ and —(CH₂)₀₋₃OC(O)N(R₃)—(CH₂)₀₋₃, —(CH₂)₀₋₃ NR₃C(O)NR₃S(O)₂—(CH₂)₀₋₃, —(CH₂)₀₋₃ NR₃C(O)NR₃C(O)—(CH₂)₀₋₃, and —(CH₂)₀₋₃—C(O)—N(R₃)—C(O)N(R₃)—(CH₂)₀₋₃ and —(C₀-C₃alkyl)(R₃)N—S(O)₂—N(R₃)—C₂-C₄alkyl-O—C₀-C₃alkyl, -and R₃R₃ₐNS(O)₂NR₃—C₂-C₄alkyl-O—C₀-C₆ alkyl-, when Y is absent, and —R₃R₃ₐNS(O)₂NR₃—C₂-C₄alkyl, when Y is absent.

Embodiment Z is another preferred embodiment according to formula (I), wherein L is preferably one of the following groups: 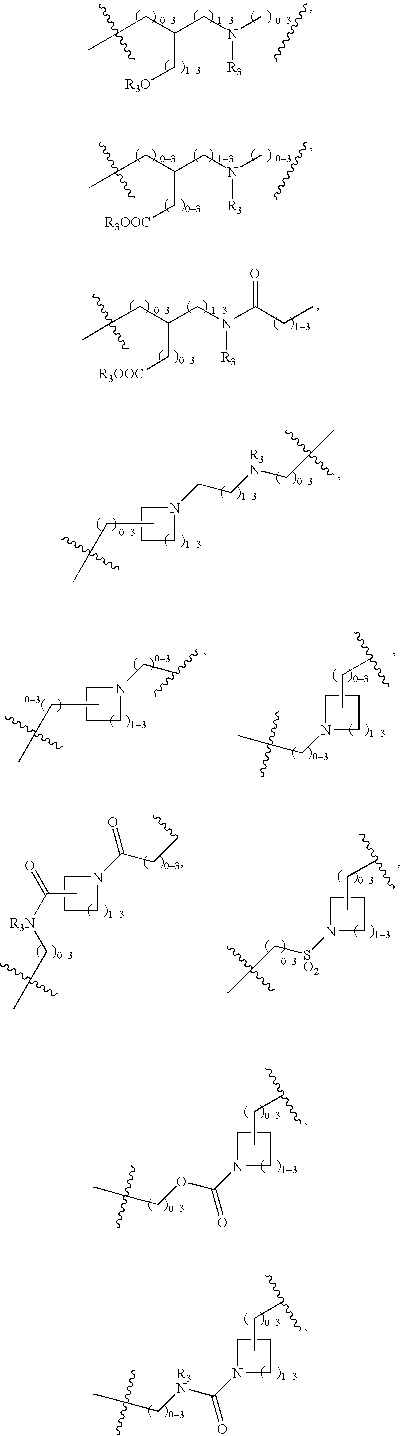 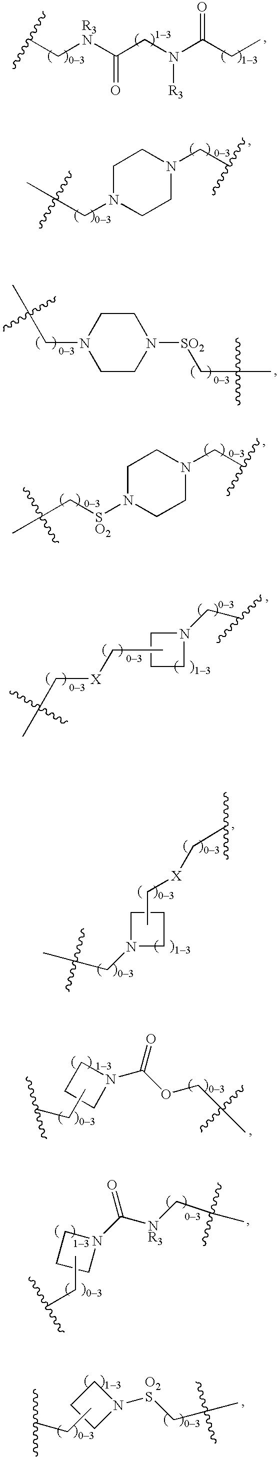 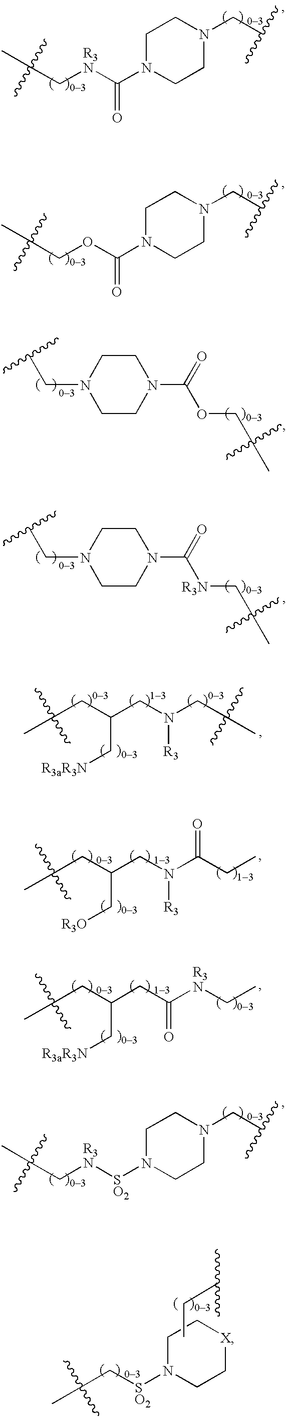 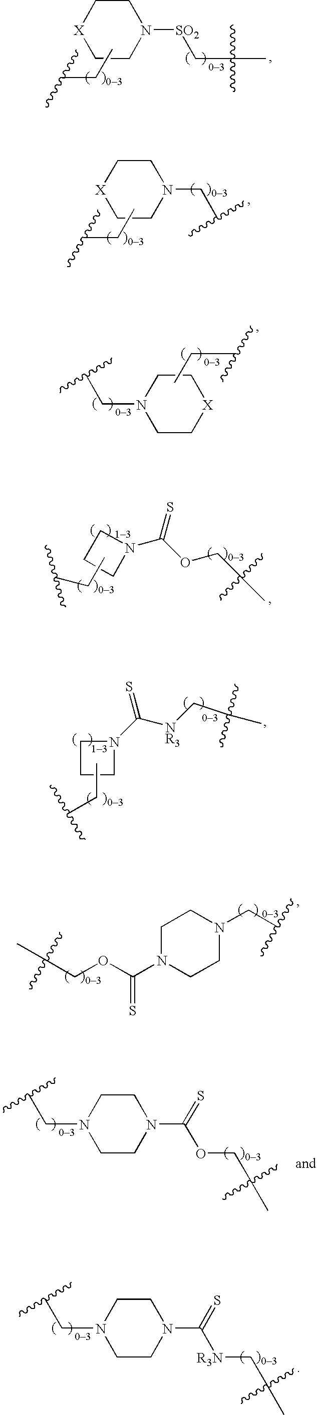
wherein A is —CH═ or —N═.

Embodiment AA is another preferred embodiment according to formula (I), wherein Y is selected from the group consisting of 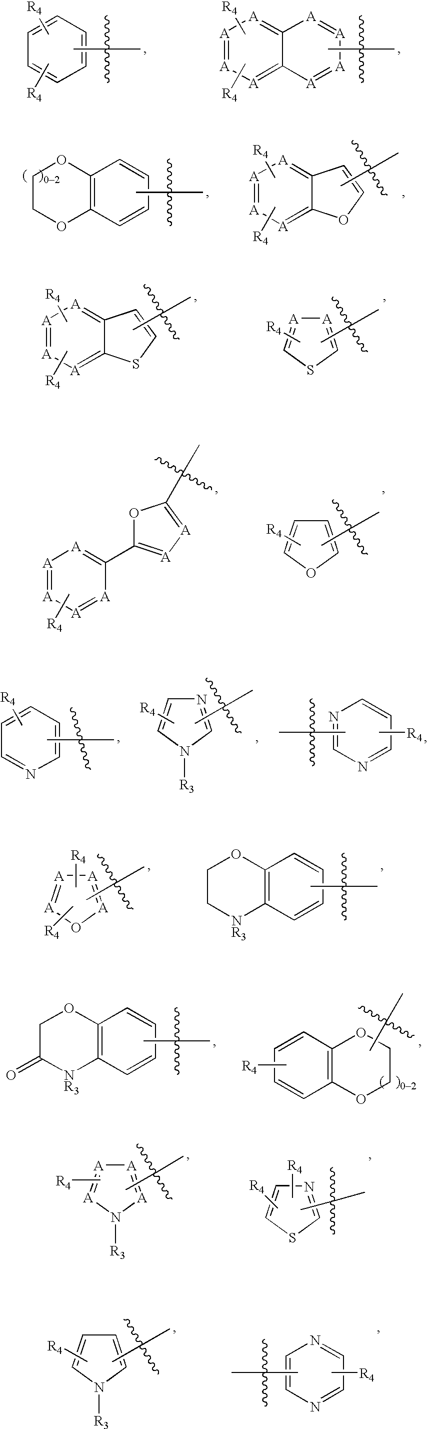 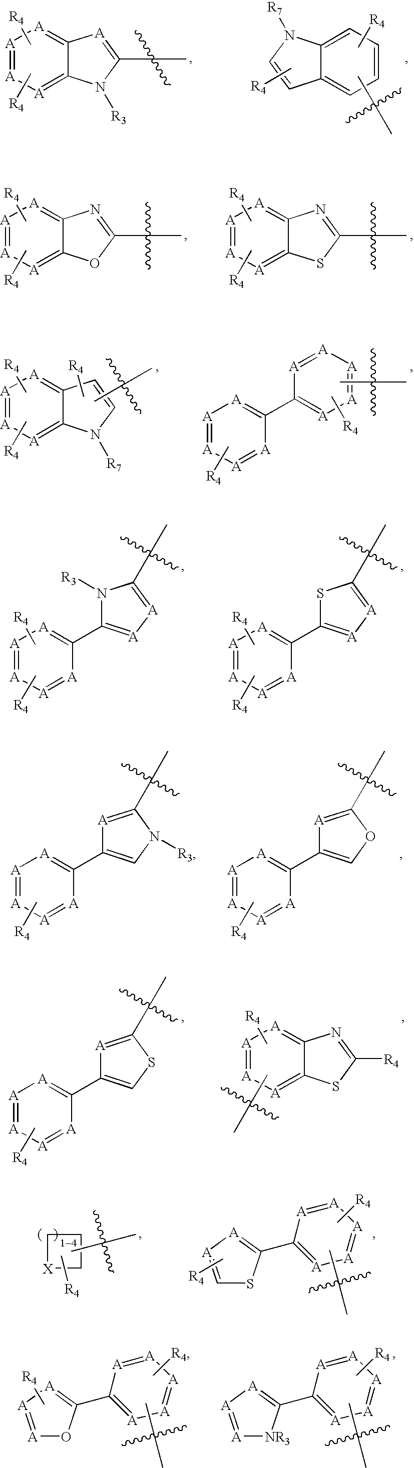 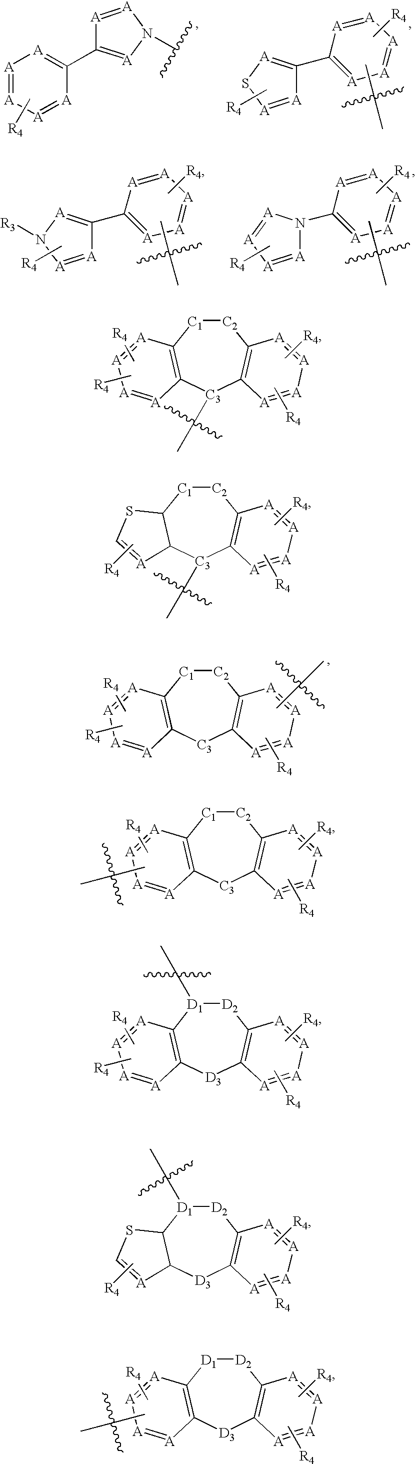 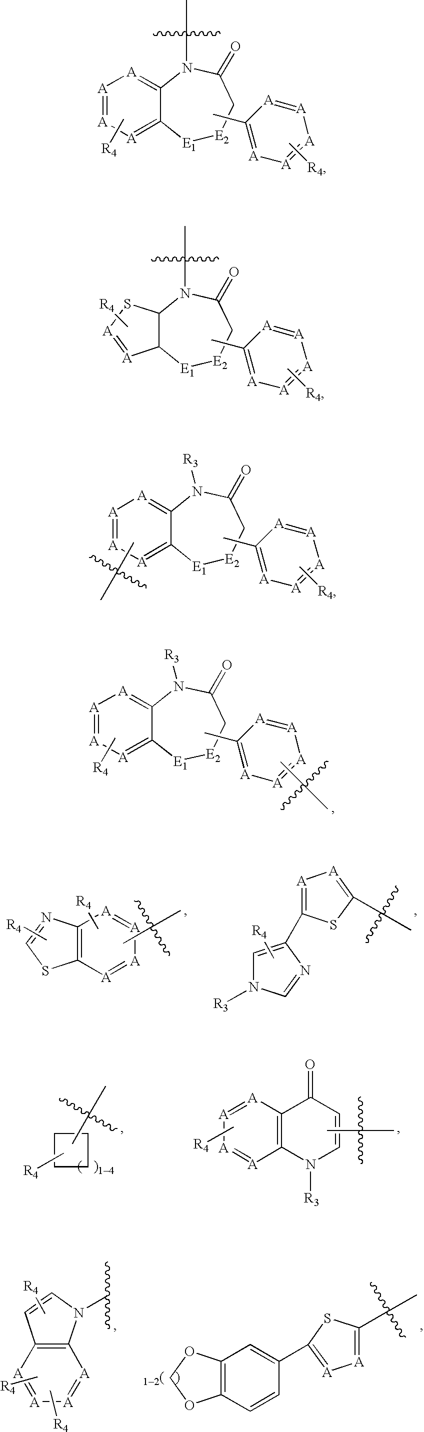 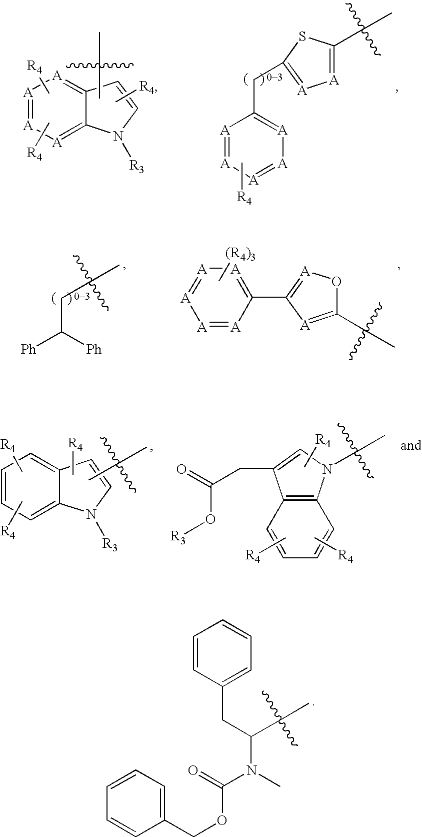
wherein A is —CH═ or —N═;
C₁ is selected from the group consisting of absent, a covalent bond, CH, CH₂, S, O, SO₂, C(O) and CR₃R₃;
C₂ is selected from the group consisting of absent, a covalent bond, CH, CH₂ and NR₃;
C₃ is selected from the group consisting of CH, N and NR₃;
D₁ is selected from the group consisting of N, CO and CH₂;
D₂ is selected from the group consisting of C, N and CH;
D₃ is selected from the group consisting of O, NR₃, SO and S;
E₁ is selected from the group consisting of S, C and N; and
E₂ is selected from the group consisting of CH, N and C(O).

Embodiment BB is another preferred embodiment according to formula (I), wherein Y-L-Z- is preferably one of the following groups: 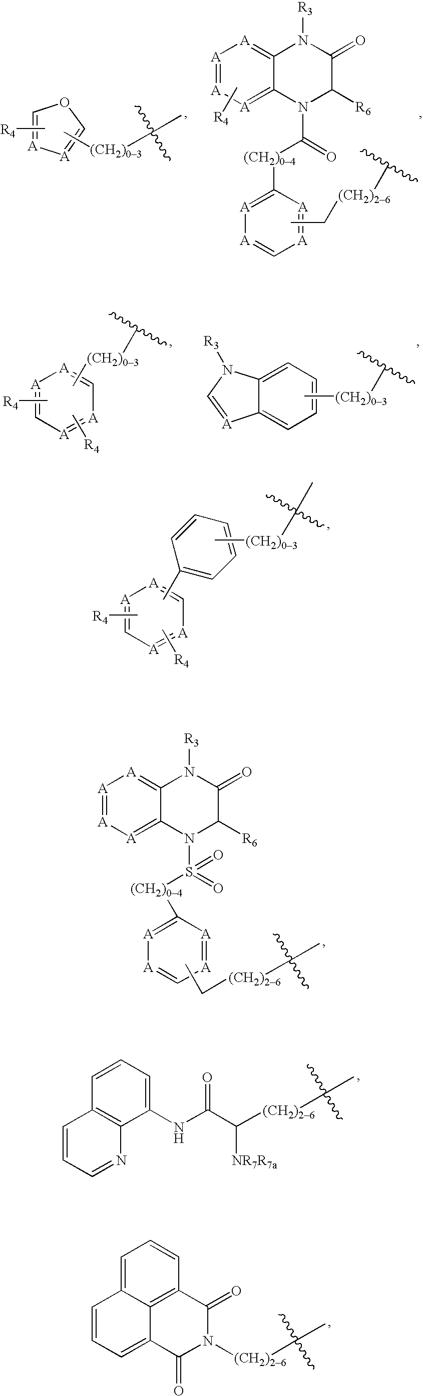 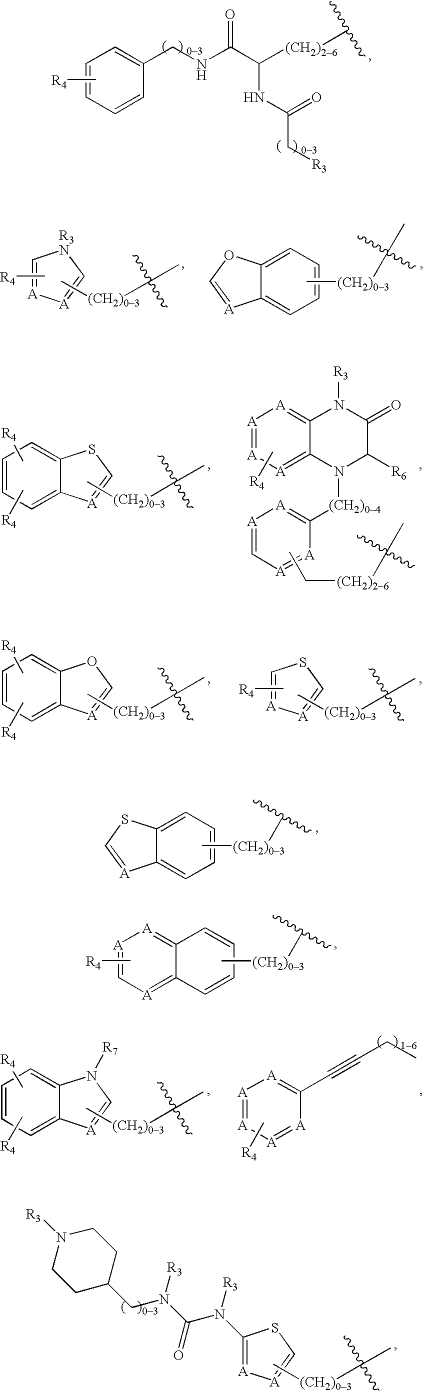 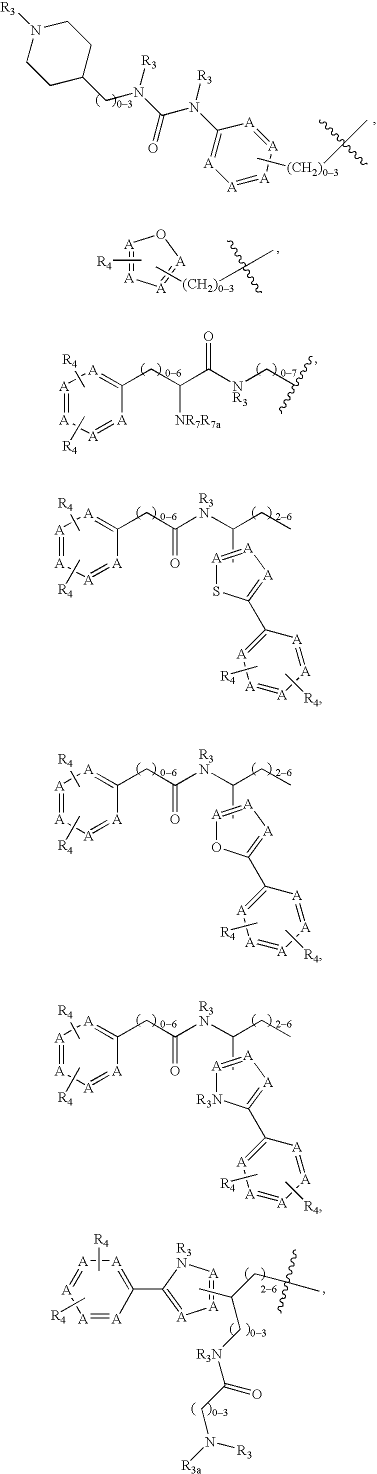 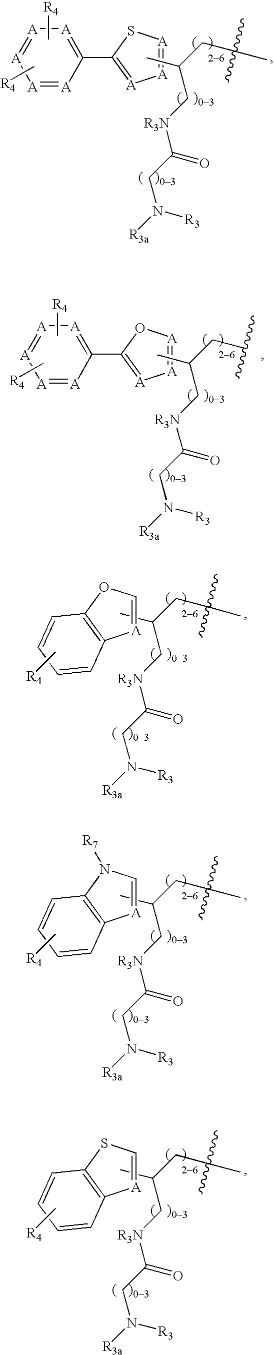 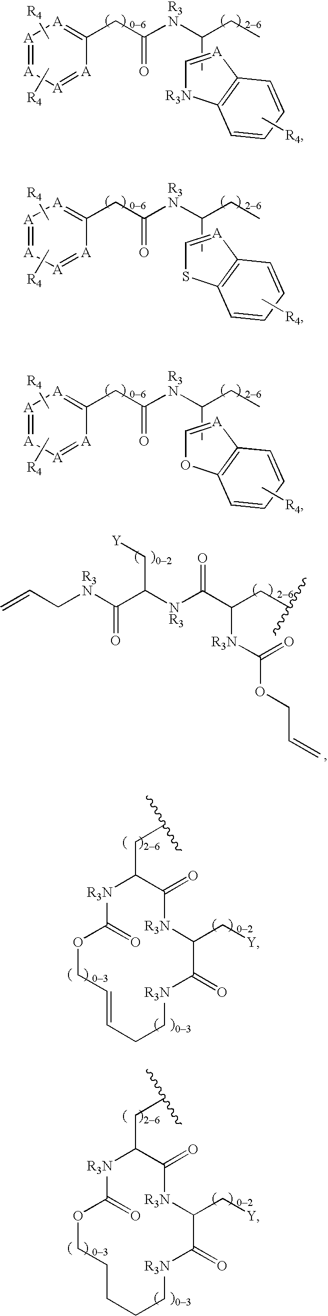 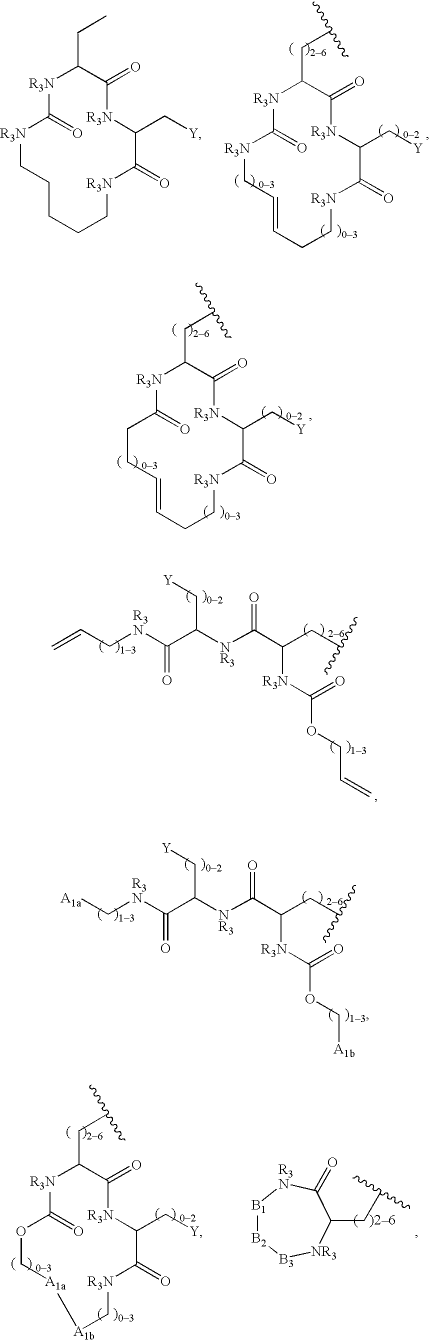 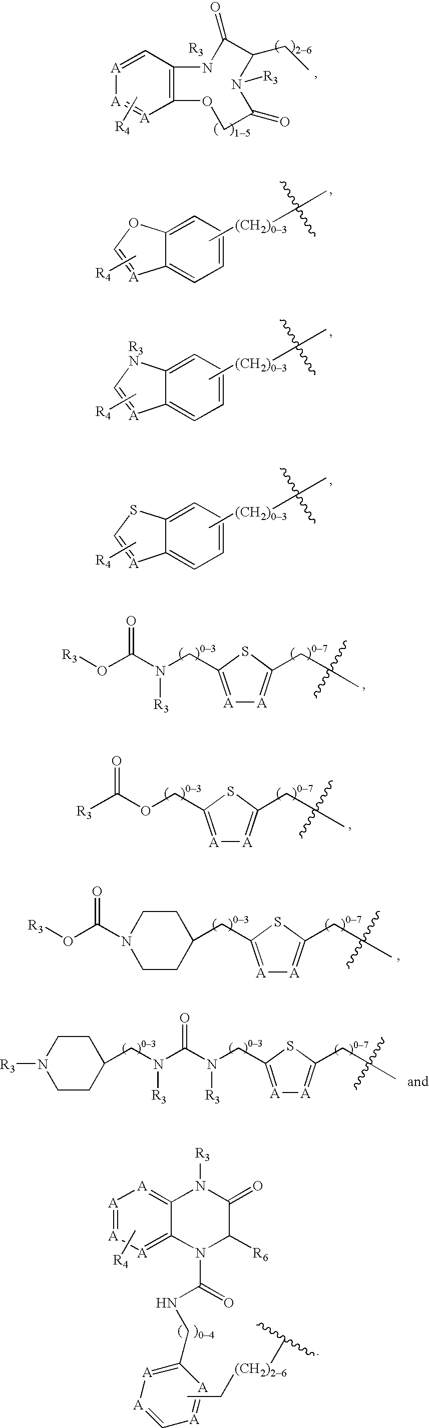
wherein A is —CH═ or —N═;
A₁ₐ and A_{1b} are independently selected from the group consisting of alkyl, alkenyl and protecting group; or
A₁ₐ and A_{1b} together via a —C₂-C₆alkylene, —C₂-C₆alkenylene or —C₂-C₆alkynylene linker, form an optionally substituted ring; and
B₁, B₂ and B₃ are each independently a natural or synthetic amino acid.

Another preferred embodiment according to formula (I), Embodiment CC, provides compounds wherein B₁, B₂ and B₃ are independently selected from the group consisting of D-Gly, L-Gly, D-Pro, L-Pro, D-Tyr, L-Tyr, D-Tyr(OR₃), L-Tyr(OR₃), D-Phe, L-Phe, D-PheR₄, L-PheR₄, D-A_{1b}, L-A_{1b}, D-Ala, L-Ala, D-ProR₃, L-ProR₃, D-Ile, L-Ile, D-Leu, L-Leu D-PheR₃, L-PheR₃, D-Pip and L-Pip.

Embodiment DD is another preferred embodiment according to formula (I), wherein L is preferably a covalent bond and Z- is preferably one of the following groups:

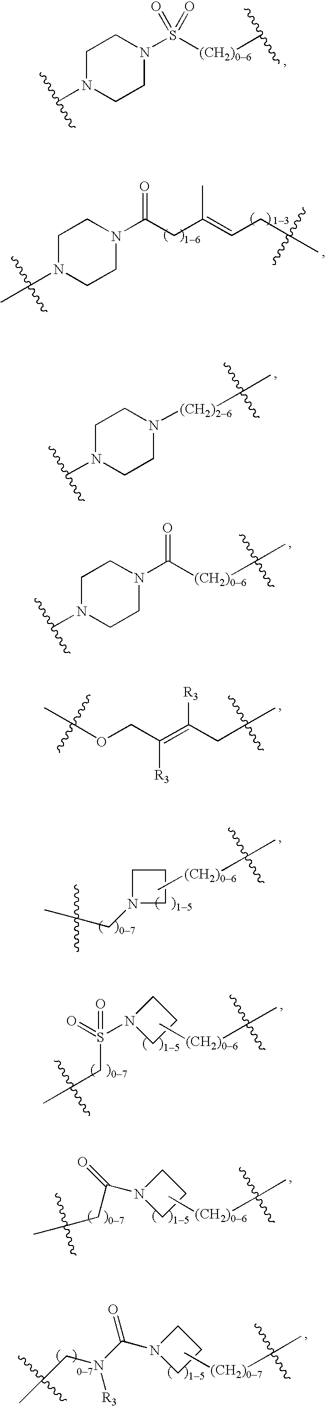

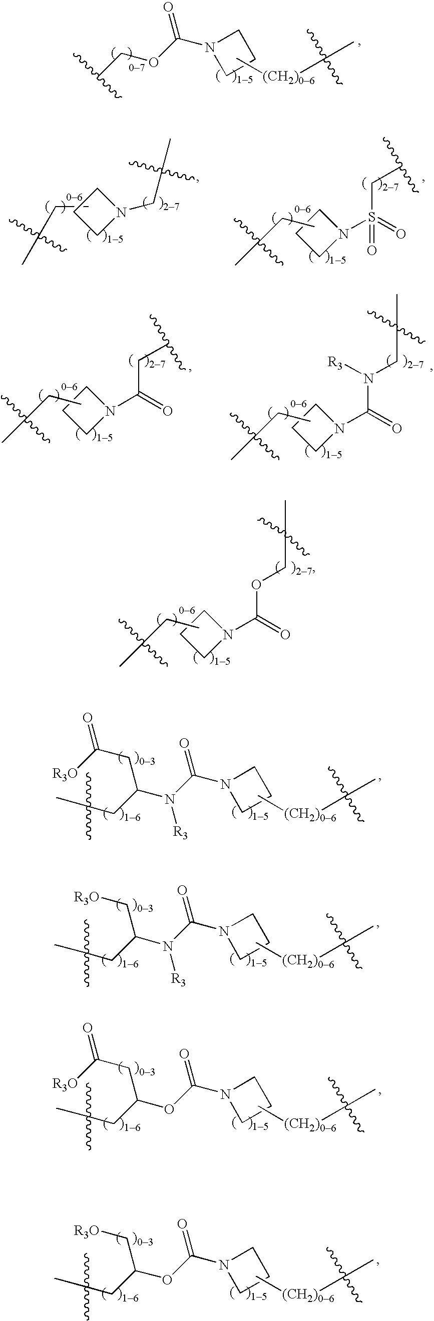

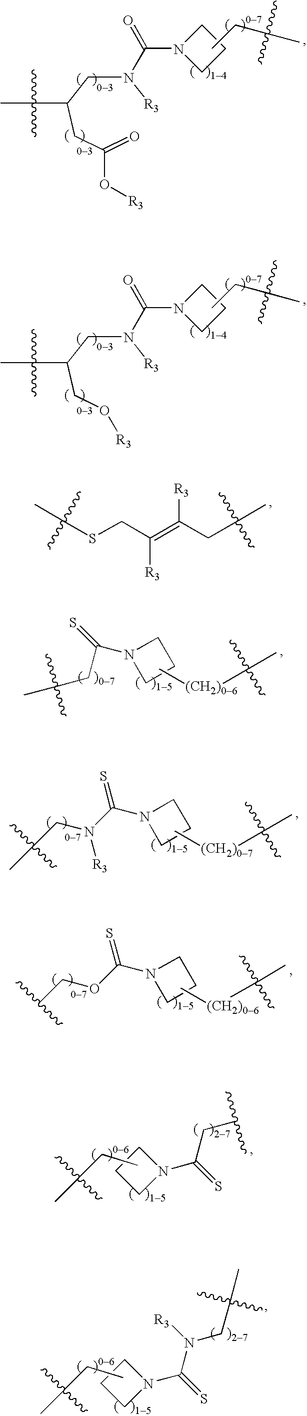

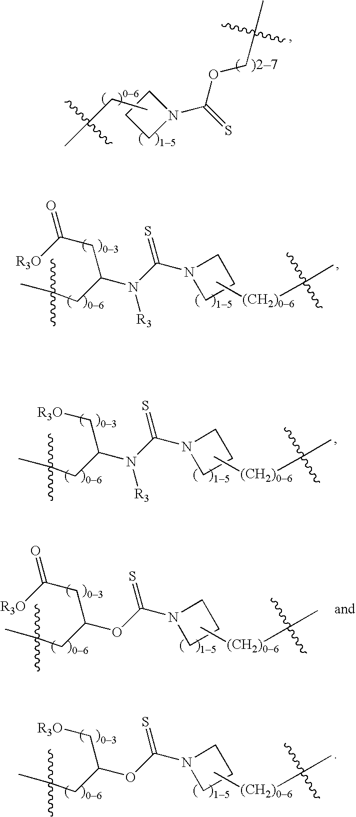

Embodiment EE is another preferred embodiment according to formula (I), wherein R₆ is preferably one of the following groups:

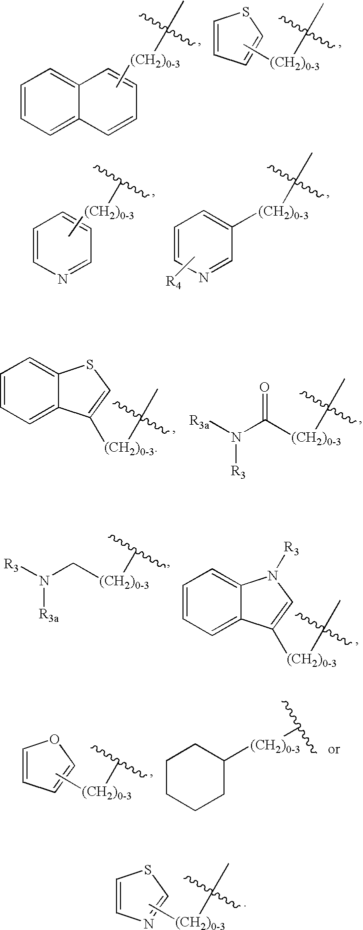

In another preferred embodiment, Embodiment FF, the invention provides compounds according to formula (I), wherein R₇ is —H, optionally substituted C₁-C₆ alkyl, —(CH₂)₂₋₄OR₃, —(CH₂)₂₋₄N(R₃)(R₃ₐ), —C(O)Ot-butyl, —C(O)O-benzyl, —(CH₂)₂-morpholinyl or —(CH₂)₂-piperazynyl.

In yet another preferred embodiment, Embodiment GG, the invention provides compounds according to formula (I), wherein then Q is —C(O)—OR₃ and X is —N(R₃)—, —C(H)₂— or —CH(OH)—. Preferably, Q is —C(O)R₃ and X is —S—, —O— or —N(R₃)—. Also preferred are compounds wherein X is —CH₂— and Q is —(CH₂)₀₋₃—X—(CH₂)₁₋₃—C(O)OR₃, —(CH₂)₀₋₃—X—(CH₂)₂₋₃—OR₃, or —(CH₂)₀₋₃—X—(CH₂)₂₋₃—N(R₃)(R₃ₐ).

In another preferred embodiment, Embodiment HH according to formula (I), the compounds are compounds wherein Y, L, Z, X, Q, R₁, R₂, R₃, R₃ₐ, R₄, R₄ₐ, R₆, R₇, and R₇ₐ are as defined in Embodiments A to GG.

Another preferred embodiment, Embodiment II provides compounds according to formula (I), wherein
W is nitrogen;
X is a covalent bond or —CH₂—;
R₁ and R₂ are —H
R₃ is —H, —OH, —C(O)—NH-aryl, —C(O)—NH₂, —C(NH₂)—C(O)—OH, NH₂, —C(NH₂)—C(O)—O—C₁-C₆ alkyl, —C(O)—OH, —C(O)—O—C₁-C₆ alkyl, aryl, heteroaryl, wherein each of the aryl and heteroaryl is optionally substituted with one or more groups selected from —OH, —CN, C₁-C₆ alkyl, —N(R₄)(R₄ₐ), halo;
Q is —C₁-C₆ alkyl-N(R₃)—C(O)—C₁-C₆ alkyl-B—(CH₂)ₙ—R₃;
B is —O—, —S(O)—, or —S—;
n is 0 or an interger from 1 to 3;
R₄ and R₄ₐ are —H; and
Y-L-Z- is aryl-C₀-C₆ alkyl-aryl-C₀-C₆ alkyl-, aryl-C₀-C₆ alkyl-aryl-C₀-C₆ alkenyl-, aryl-C₀-C₆ alkyl-aryl-C₀-C₆ alkynyl-, aryl-C₀-C₆ alkenyl-aryl-C₀-C₆ alkyl-, aryl-C₀-C₆ alkenyl-aryl-C₀-C₆ alkenyl-, aryl-C₀-C₆ alkenyl-aryl-C₀-C₆ alkynyl-, aryl-C₀-C₆ alkynyl-aryl-C₀-C₆ alkyl-, aryl-C₀-C₆ alkynyl-aryl-C₀-C₆ alkenyl-, aryl-C₀-C₆ alkynyl-aryl-C₀-C₆ alkynyl-.

Another preferred embodiment, Embodiment JJ provides compounds according to Embodiment II of the formula

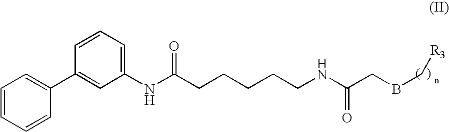

or a pharmaceutically acceptable salt thereof, wherein B, n and R₃ are any one of the following combination:

| n | B | R₃ | n | B | R₃ | n | B | R₃ |
|---|---|---|---|---|---|---|---|---|
| 1 | O | 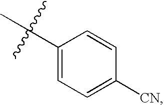 | 1 | O | 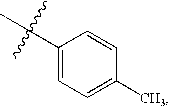 | 1 | O | 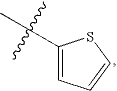 |
| 1 | O | 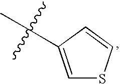 | 1 | S | 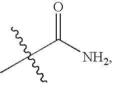 | 0 | S | 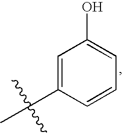 |
| 1 | O | 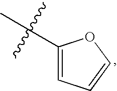 | 1 | S | 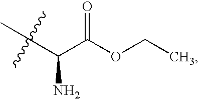 | 0 | S | 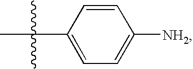 |
| 1 | O | 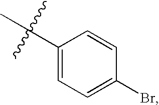 | 1 | S | 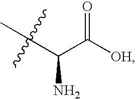 | 0 | S | 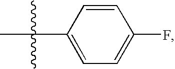 |
| 1 | O | 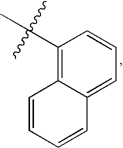 | 2 | S | —NH2 | 0 | S | 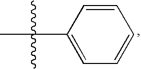 |
| 1 | S O | 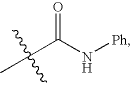 | 0 | S | 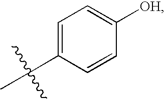 | 0 | S | 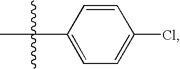 |
| 2 | S | 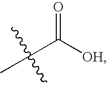 | 1 | S O | 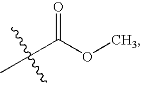 | 0 | S | 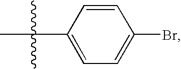 |
| 2 | S O | 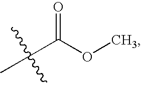 | 2 | S | 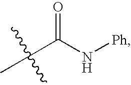 | 0 | S | 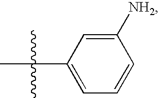 |
| 3 | S | —OH, | 2 | S O | 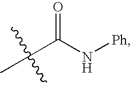 | 0 | S | 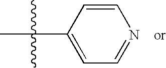 |
| 2 | S | —OH, | | | | 0 | S | 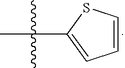 |

Another preferred embodiment, Embodiment KK provides compounds according to formula (I), wherein
W is nitrogen;
X is a covalent bond or —CH₂;
R₁ and R₂ are —H;
R₃ is H, aryl or heteroaryl, wherein each of the aryl and heteroaryl is optionally substituted with one or more groups selected from —CN, —S(O)—C₁-C₆ alkyl, C₁-C₆ alkyl, or halo;
Q is —C₁-C₆ alkyl-N(R₃)—C(O)—C₁-C₆ alkyl-B—C₁-C₆ alkyl-R₃;
B is —S—, —S(O)— or —O—; and
Y-L-Z- is aryl-C₀-C₆ alkyl-aryl-C₀-C₆ alkyl-.

Another preferred embodiment, Embodiment LL provides compounds according to Embodiment KK of the formula 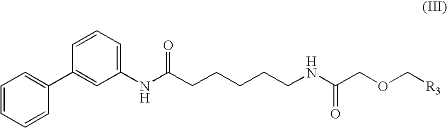
or a pharmaceutically acceptable salt thereof, wherein R₃ is selected from the group consisting of

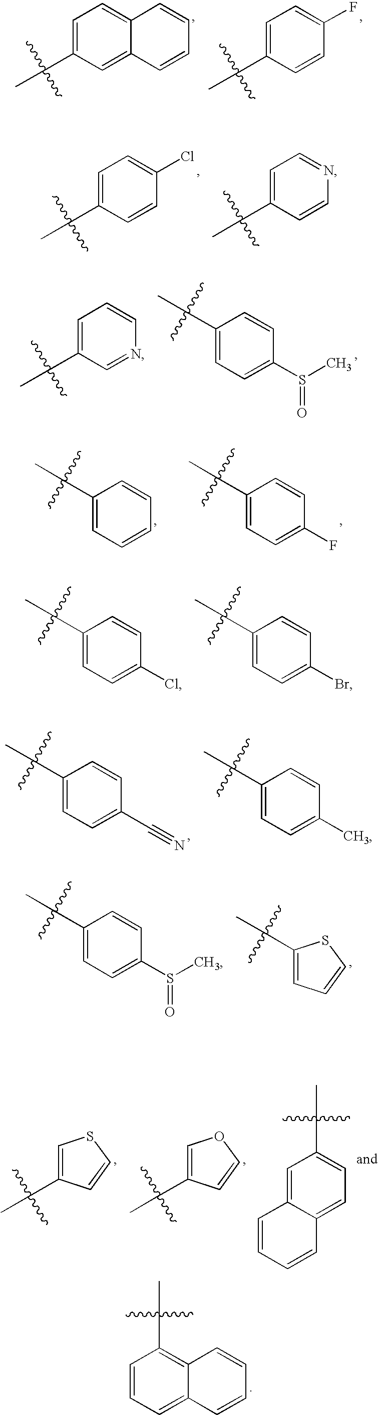

Another preferred embodiment, Embodiment MM provides compounds according to formula (I), wherein
W is nitrogen;
X is a covalent bond or —CH₂—;
R₁ and R₂ are —H;
R₃ is —H, —C₁-C₆ hydroxyalkyl-C(O)—OH, —C₁-C₆ alkyl-S—C₁-C₆ alkyl-C(NH₂)—C(O)—OR₄, —C₁-C₆ alkyl-S(O)—C₁-C₆ alkylaryl, —C₁-C₆ alkyl-S—C₀-C₆ alkylaryl, —C₁-C₆ alkyl-S—C₀-C₆ alkylheteroaryl, —C₁-C₆ alkyl-S—C₁-C₆ alkyl-OH, —C₁-C₆ alkyl-S—C₁-C₆ alkyl-C(O)—OH, —C₁-C₆ alkyl-S—C₁-C₆ hydroxyalkyl-C(O)—O—C₁-C₆ alkyl, —C₁-C₆ alkyl-S—C₁-C₆ alkyl-C(O)—O—C₁-C₆ alkyl, —C₁-C₆ alkyl-S(O)—C₁-C₆ alkyl-C(O)—OR₄, —C₁-C₆ alkyl-S—C₁-C₆ alkyl-C(O)—N(R₄)(R₄ₐ), —C₁-C₆ alkyl-S(O)—C₁-C₆ alkyl-C(O)—N(R₄)-aryl, —C₁-C₆ alkyl-S—C₁-C₆ alkyl-N(R₄)(R₄ₐ), and —C₁-C₆-alkylheteroaryl, wherein each of the aryl and heteroaryl is optionally substituted with one or more groups selected from oxo, —OH, —N(R₄)(R₄ₐ), halo, —S—C₁-C₆ alkyl, or —S(O)—C₁-C₆ alkyl;
Q is —C₁-C₆ alkyl-N(R₃)—C(O)—R₃;
R₄ and R₄ₐ are independently —H, C₁-C₆ alkyl, or aryl; and
Y-L-Z- is aryl-C₀-C₆ alkyl-aryl-C₀-C₆ alkyl-.

Another preferred embodiment, Embodiment NN provides compounds according to Embodiment MM of the formula 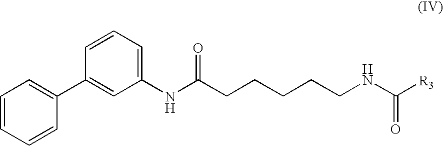
or a pharmaceutically acceptable salt thereof, wherein R₃ is selected from the group consisting of

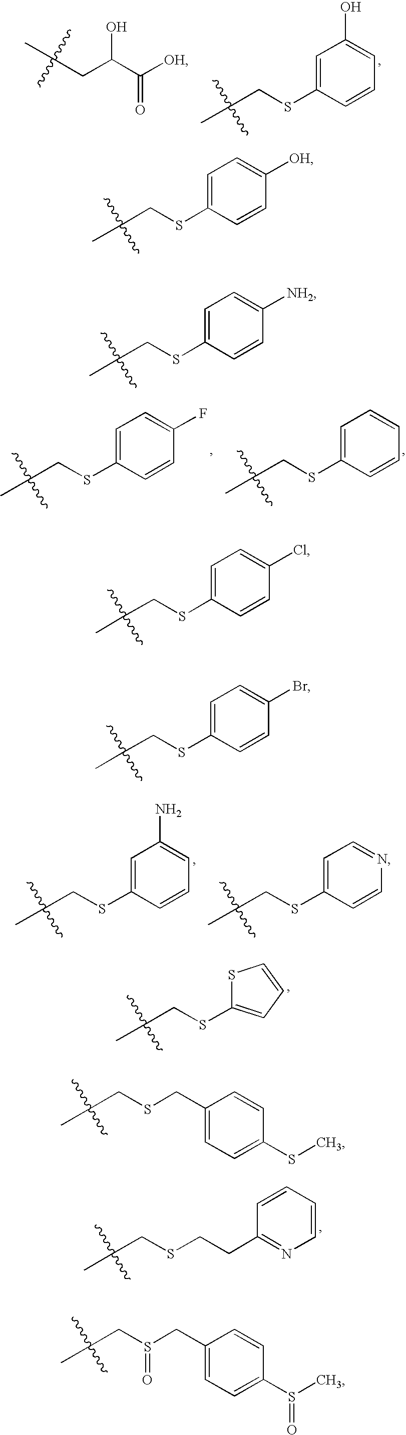

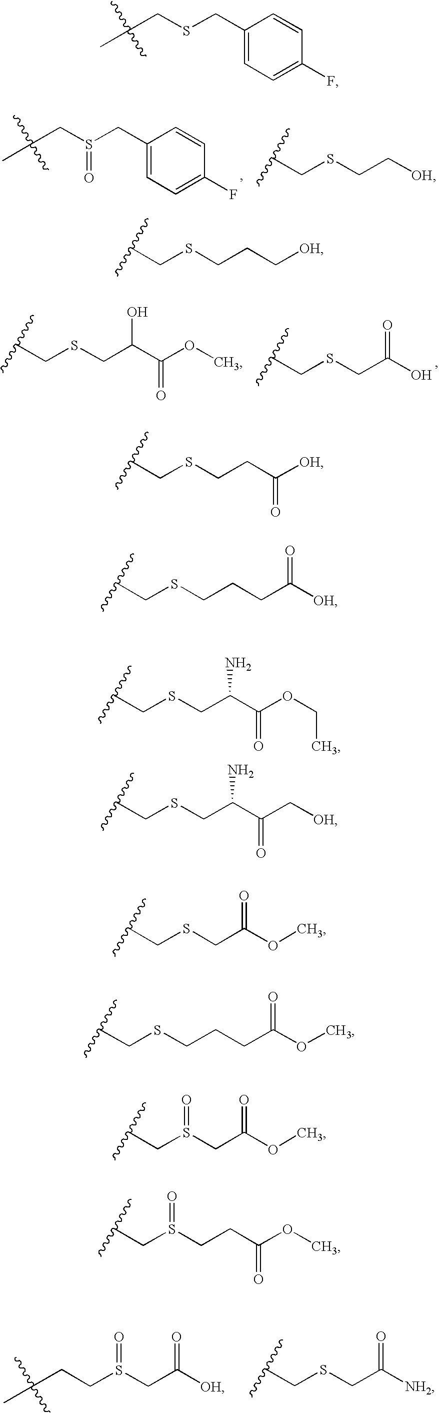

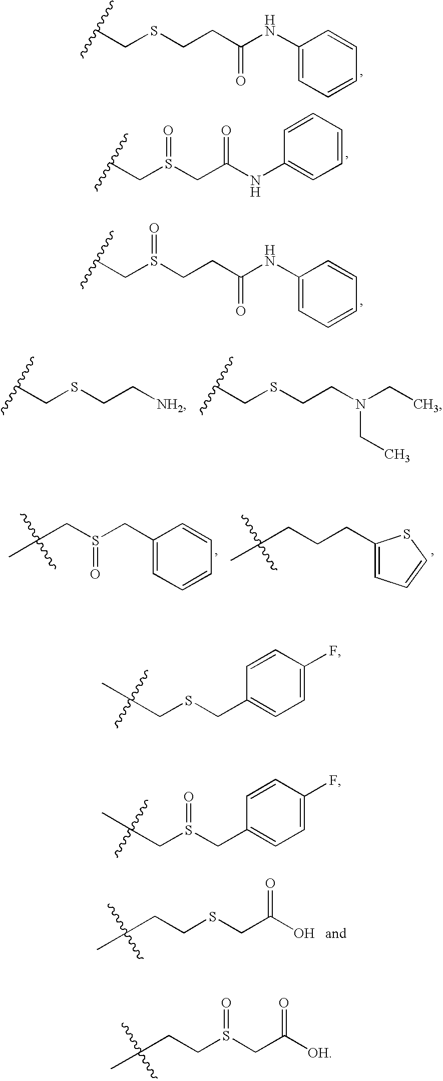

Another preferred embodiment, Embodiment OO provides compounds according to formula (I), wherein
W is nitrogen;
X is —S—;
R₁ and R₂ are —H;
R₃ is —H;
Q is —C₁-C₆-alkyl-C(O)—OR₃; and
Y-L-Z- is heteroaryl-C₀-C₆ alkyl- or heteroaryl-C₀-C₆ alkyl-heteroaryl-C₀-C₇ alkyl-aryl-C₀-C₆-alkyl, wherein each of the aryl and heteroaryl is optionally substituted with one or more groups selected from oxo or halo.

Another preferred embodiment, Embodiment PP provides compounds according to Embodiment OO that is one of the following structures:

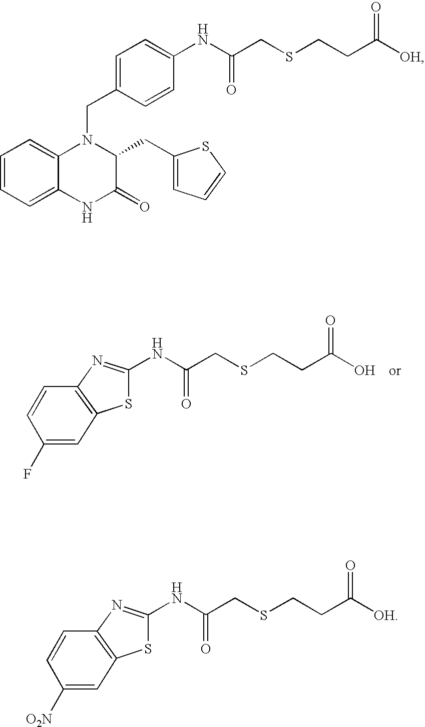

Embodiment QQ provides compounds according to formula (I) having the formula 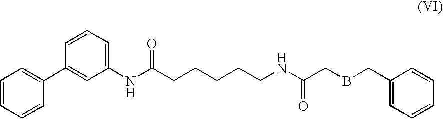
wherein B is —S— or —S(O)—.

Another preferred embodiment, Embodiment RR, provides compounds according to formula (I), wherein
W is nitrogen;
X is —S—;
R₁ and R₂ are —H;
R₃ is —H or C₁-C₆ alkyl;
R₄ is C₁-C₆ alkyl-OR₃;
Q is —C₁-C₆-alkyl-C(O)—OR₃, —C₁-C₆-alkyl-N(R₃)(R₃ₐ), C₁-C₆ alkyl substituted with —OH; and
Y-L-Z- is heteroaryl-C₁-C₆ alkyl-N(R₄)—C₁-C₆-alkyl-aryl-C₀-C₆ alkyl- or heteroaryl-C₀-C₆ alkyl-heteroaryl-C₀-C₇ alkyl-aryl-C₀-C₆-alkyl, wherein each of the aryl and heteroaryl is optionally substituted with one or more groups selected from oxo.

Another preferred embodiment, Embodiment SS, provides compounds according to Embodiment RR that is one of the following structures:

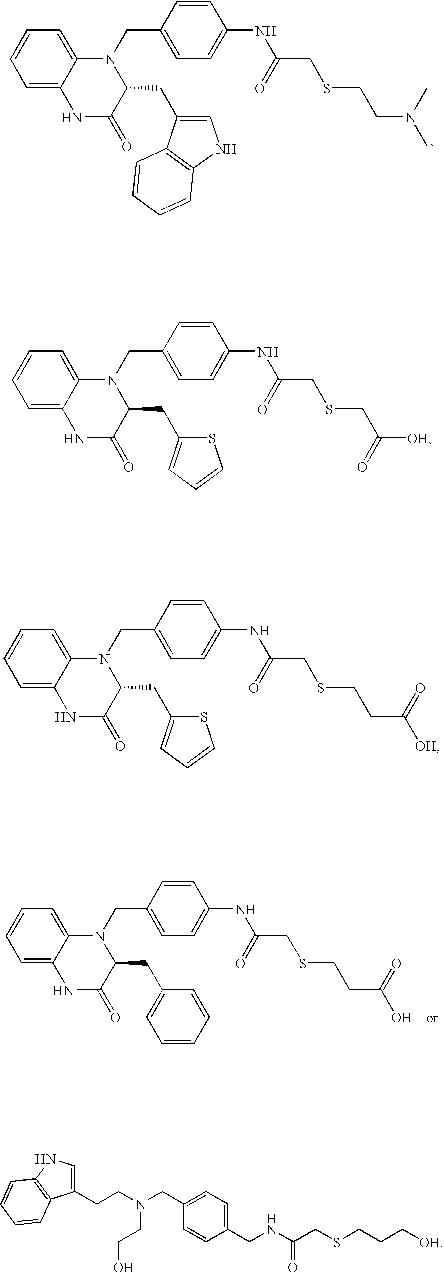

Another preferred embodiment, Embodiment TT, provides compounds according to formula (I), wherein

W is nitrogen;

X is a covalent bond or —CH₂—;

R₁ and R₂ are independently —H, —N(H)—C(O)—O—C₁-C₆ alkyl or —N(H)—C(O)—O-benzyl;

R₃ is —H or —C₁-C₆ alkyl-O—C₀-C₆ alkylaryl;

Q is —C₁-C₆ alkyl-N(R₃)—C(O)—R₃; and

Y-L-Z- is heteroaryl-C₀-C₆ alkyl-.

Another preferred embodiment, Embodiment UU, provides compounds according to Embodiment TT that is one of the following structures:

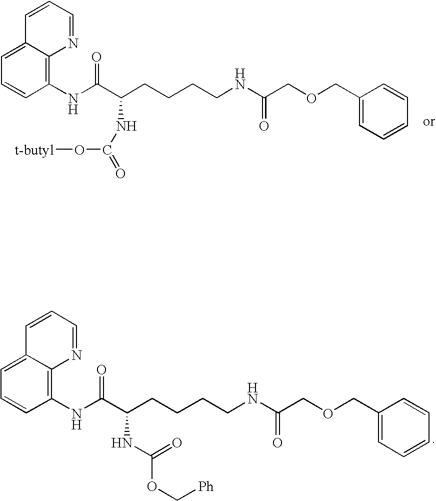

Another preferred embodiment, Embodiment TT, provides compounds according to formula (I), wherein

W is nitrogen;

Q is —C₁-C₆-alkyl-C(O)—OR₃; and

Y-L-Z- is heteroaryl-C₀-C₆ alkyl-, wherein the heteroaryl is optionally substituted with one or more groups selected from C₁-C₆ alkoxy, halo or —NO₂.

Another preferred embodiment, Embodiment WW, provides compounds according to Embodiment VV that is one of the following structures:

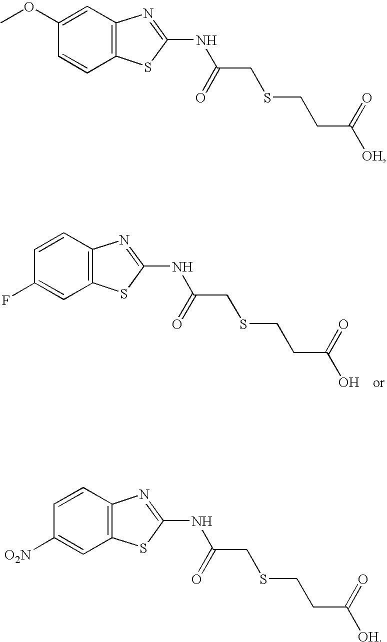

Another preferred embodiment, Embodiment XX, provides compounds according to Formula (I) wherein
W is nitrogen;
X is selected from the group consisting of S, O, SO and SO₂;
R₁ and R₂ are H or halogen;
Q is selected from the group consisting of aryl-NH₂, C₁-C₆alkyl-aryl, C₁-C₆alkyl-heteroaryl, C₁-C₆alkyl-CN, wherein the alkyl, aryl and heteroaryl are each independently optionally substituted;
Z is selected from the group consisting of —C₁-C₆alkyl-, —C₁-C₈heteroalkyl-, —C₀-C₆alkyl-aryl-C₀-C₃alkyl-X—C₀-C₃alkyl-, —C₀-C₆alkyl-heteroaryl-C₀-C₃alkyl-X—C₀-C₃alkyl-, —C₀-C₆alkyl-heteroaryl-C₂-C₆heteroalkyl-, —C₀-C₃alkyl-X—C₀-C₃alkyl-, —C₀-C₆alkyl-aryl-C₃-C₆alkynyl- and —C₀-C₆alkyl-aryl-C₃-C₆alkenyl-, wherein the alkyl, heteroalkyl, aryl, heteroaryl and alkenyl are each independently optionally substituted;
L is selected from the group consisting of —C₀-C₆alkyl-S(O)₂—N(R₃)—C₀-C₆alkyl-, —C₀-C₆alkyl-O—C(O)—N(R₃)—C₀-C₃alkyl-, —C₀-C₆alkyl-N(R₃)—S(O)₂—C₀-C₃alkyl-, -heterocyclyl-C(O)—C₀-C₃alkyl-, —C₀-C₆alkyl-N(R₃)—C(O)—C₀-C₃alkyl-, —C₀-C₆alkyl-C(O)—N(R₃)—C₀-C₃alkyl-, covalent bond, —C₀-C₆alkyl-N(R₃)—C₀-C₃alkyl-, —C₀-C₆alkyl-, —S(O)₂—N(R₃)—C₀-C₃alkyl-aryl-C₀-C₃alkyl-C(O)—N(R₃)—C₁-C₃alkyl-, —C₀-C₆alkyl-S(O)₂—C₀-C₃alkyl-, —C₀-C₃alkyl-S(O)₂—N(R₃)—C₀-C₃alkyl-heterocyclyl-C₀-C₃alkyl-, —C₀-C₃alkyl-O—C(O)—N(R₃)—C₀-C₃alkyl-heterocyclyl-C₀-C₃alkyl-, —C₀-C₃alkyl-C(O)—N(R₃)—C₀-C₃alkyl-heterocyclyl-C₀-C₃alkyl-, —C₀-C₃alkyl-N(R₃)—C(O)—O-heterocyclyl-C₀-C₃alky- and —C₀-C₆alkyl-C(O)—N(R₃)—C(O)—C₀-C₃alkyl-, wherein the alkyl, heterocyclyl and aryl are each independently optionally substituted; and
Y is selected from the group consisting of aryl-aryl, aryl, heterocyclyl-aryl, heteroaryl, heteroaryl-aryl, heterocyclyl, alkylaryl, alkylheterocyclyl, aryl-alkylheterocyclyl, heterocyclyl-alkyl-aryl, alkyl, heteroaryl-heteroaryl and heterocyclyl-heteroaryl, wherein each said Y is independently optionally substituted.

Another preferred embodiment, Embodiment YY, provides compounds according to Embodiment XX wherein Q is C₁-C₆alkyl-heteroaryl, wherein said C₁-C₆alkyl is optionally substituted with —CH₂—C(O)—O—C₁-C₆alkyl.

Another preferred embodiment, Embodiment ZZ, provides compounds according to Formula (I) wherein
W is nitrogen;
X is —O— or —S—;
R₁, R₂ are H;
Q is selected from the group consisting of C₀-C₆alkyl-aryl and heteroaryl, wherein said alkyl, aryl and heteroaryl are independently optionally substituted;
Y-L-Z is selected from the group consisting of aryl-C₀-C₆alkyl-C(O)—N(R₃)—C₁-C₇alkyl-, (R₃)(R₃ₐ)N—C₀-C₆alkyl-C(O)—N(R₃)—C₁-C₇alkyl-, aryl-C₀-C₆alkyl-O—C(O)—N(R₃)—C₁-C₇alkyl-, C₀-C₃alkyl-heteroaryl-C₀-C₃alkyl-N(R₃)—C(O)—C₁-C₇alkyl-, C₁-C₇alkyl-N(R₃)—C(O)—C₀-C₆alkyl-, heteroaryl-C₀-C₆alkyl-C(O)—N(R₃)—C₁-C₇alkyl-, wherein each of said aryl, alkyl and heteroaryl are independently optionally substituted.

Another preferred embodiment, Embodiment AAA, provides compounds according to Embodiment ZZ, wherein said C₁-C₇alkyl is optionally substituted with a substituent selected from the group consisting of heteroaryl-aryl, —C(O)—N(R₃)-heteroaryl, —N(R₃)—C(O)—O-alkenyl, heteroaryl and —N(R₃)—C(O)—O—C₀-C₃alkyl-aryl, and said C₀-C₃alkyl is optionally substituted with —C(O)—N(R₃)alkenyl.

Another preferred embodiment, Embodiment BBB, provides compounds according to Formula (I) wherein
W is nitrogen;
X is —O— or —S—;
R₁ and R₂ are H;
Q is alkyl-aryl; and
Y-L-Z is selected from the group consisting of aryl-C₀-C₃alkyl-N(R₃)—C(O)—C₁-C₇alkyl-, A₂ₐ-aryl-C₀-C₃alkyl-N(R₃)—C(O)—C₁-C₇alkyl- and heteroaryl-C₀-C₃alkyl-N(R₃)—C(O)—C₁-C₇alkyl-, wherein said aryl and heteroaryl are each independently optionally substituted, and wherein
said C₀-C₃alkyl is optionally substituted with —C(O)—N(R₃)—C₁-C₆alkyl-A₁ₐ or —C(O)—N(R₃)—C₀-C₆alkyl-C(O)-A₂ₐ; and
said C₁-C₇alkyl is optionally substituted with a substituent selected from the group consisting of —N(R₃)—C(O)—O—C₁-C₃alkyl-A_{1b}, —N(R₃)—C(O)—C₁-C₇alkyl-O-A_{2b}, —N(R₃)—C(O)-heteorcyclyl-A_{2b} and —N(R₃)—C(O)—C₂-C₇alkenyl-O-A_{2b}, wherein
A₁ₐ and A_{1b} optionally together via a C₂-C₆alkylene, C₂-C₆alkenylene or C₂-C₆alkynylene linker, form an optionally substituted ring system; and
A₂ₐ and A_{2b} together are a covalent bond and are attached to form a ring, or
Y-L-Z is B₂—B₁—N(R₃)—C(O)—C₁-C₇ alkyl-, wherein the C₁-C₇ alkyl is optionally substituted with —NR₃—B₃ and the amine of B₃ is conected with the acid of B₂ to form a peptide bond; wherein
B₁, B₂ and B₃ are each independently a natural or synthetic amino acid.

Another preferred embodiment, Embodiment CCC, provides compounds according to Formula (I) wherein
W is nitrogen;
X is —O—;
R₁ and R₂ are H;
Q is optionally substituted alkyl-aryl;
Z is optionally substituted C₁-C₈alkyl;
L is selected from the group consisting of —C₀-C₃alkyl-N(R₃)—C(O)-heterocyclyl-C₀-C₃alkyl-, —C₀-C₃alkyl-N(R₃)—C(S)-heterocyclyl-C₀-C₃alkyl-, —C₀-C₇alkyl-heterocyclyl-C₀-C₃alkyl-, —C₀-C₃alkyl-O—C(O)-heterocyclyl-C₀-C₃alkyl-, C₀-C₃alkyl-S(O)₂-heterocyclyl-C₀-C₃alkyl- and —C₀-C₃alkyl-C(O)-heterocyclyl-C₀-C₃alkyl, wherein said alkyl and heterocyclyl are independently optionally subsituted; and
Y is selected from the group consisting of heteroaryl, aryl, cycloalkyl and heteroaryl-aryl, each of which is optionally substituted.

Another preferred embodiment, Embodiment DDD, provides compounds according to Formula (I) wherein
W is nitrogen;
X is —O—;
R₁ and R₂ are H;
Q is optionally substituted alkyl-aryl;
Z is optionally substituted —C₀-C₆alkyl-heteroaryl-C₀-C₆alkyl and optionally substituted C₁-C₈alkyl;
L is selected from the group consisting of —C₀-C₆alkyl-S(O)₂-heterocyclyl-C₀-C₃alkyl, covalent bond, —C₀-C₆alkyl-, —C₀-C₆alkyl-O—C(O)—N(R₃)—C₀-C₃alkyl-, —C₀-C₆alkyl-C(O)—O—, —C₀-C₆alkyl-N(R₃)—C(O)-heterocyclyl-C₀-C₃alkyl-, —C₀-C₃alkyl-heteroaryl-C₀-C₃alkyl-N(R₃)—C₀-C₃alkyl-, —C₀-C₆alkyl-N(R₃)—C(O)—N(R₃)—C₀-C₃alkyl-, —C₀-C₆alkyl-C(O)—N(R₃)—C₀-C₃alkyl-, —C₀-C₆alkyl-S(O)₂—N(R₃)—C₀-C₃alkyl-, —C₀-C₆alkyl-C(O)—N(R₃)—C(O)—N(R₃)—C₀-C₆alkyl- and —C₀-C₆alkyl-S(O)₂—C₀-C₃alkyl, wherein the alkyl, heterocyclyl, heteroaryl are independently optionally substituted; and
Y is selected from the group consisting of aryl, alkylaryl, heteroaryl, aryl-heterocyclyl, aryl-heteroaryl, alkyl and heterocyclyl, each of which is independently optionally substituted.

Another preferred embodiment, Embodiment EEE, provides compounds according to Formula (I) wherein
W is nitrogen;
X is —O—;
R₁ and R₂ are H;
Q is optionally substituted alkyl-aryl;
Z is selected from the group consisting of —C₀-C₆alkyl-aryl-C₀-C₆alkyl-, —C₀-C₆alkyl-aryl-C₀-C₃alkyl-X—C₀-C₃alkyl-, —C₀-C₆alkyl-aryl-C₃-C₆alkenyl-C₀-C₃alkyl and —C₀-C₆alkyl-aryl-C₃-C₆alkynyl-C₀-C₃alkyl, wherein the alkyl, aryl and alkynyl are each independently optionally substituted;
L is selected from the group consisting of —C₀-C₃alkyl-N(R₃)—C₀-C₃alkyl, —C₀-C₃alkyl-heterocyclyl-C₀-C₃alkyl-O—C₀-C₃alkyl-, —C₀-C₆alkyl-S(O)₂—N(R₃)—C₀-C₆alkyl-, —C₀-C₆alkyl-O—C(O)—, —C₀-C₆alkyl-C(O)—N(R₃)—S(O)₂—C₀-C₃alkyl- and —C₀-C₆alkyl-N(R₃)—S(O)₂—C₀-C₃alkyl, wherein said alkyl and heterocyclyl are each independently optionally substituted; and
Y is selected from the group consisting of heteroaryl, aryl, heteroaryl-hetercyclyl, alkyl, aryl-heterocyclyl and cycloalky, each of which is independently optionally substituted.

Another preferred embodiment, Embodiment FFF, provides compounds according to Formula (I) wherein
W is nitrogen;
X is —O—;
R₁ and R₂ are H;
Q is optionally substituted alkyl-aryl;
Z is optionally substituted C₁-C₆alkyl;
L is —C₀-C₃alkyl-N(R₃)—C(O)—C₀-C₃alkyl-heterocyclyl-C(O)—C₀-C₃alkyl, wherein the alkyl and heterocyclyl are independently optionally substituted; and
Y is selected from the group consisting of aryl-aryl, alkyl-heteroaryl, aryl and heteroaryl, each of which is independently optionally substituted.

Another preferred embodiment, Embodiment GGG, provides compounds according to Formula (I) wherein 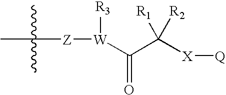
is the structure

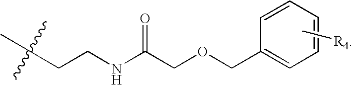

In the second aspect, the invention provides a composition comprising a compound according to the first aspect and Embodiments A to GGG and a pharmaceutically acceptable carrier.

In the third aspect, the invention provides a method of inhibiting histone deacetylase. In one embodiment, the method comprising contacting the histone deacetylase with an inhibiting effective amount of a compound according to the first aspect and Embodiments A to GGG. In a further embodiment of the third aspect, the method comprises contacting the histone deacetylase with an inhibiting effective amount of a composition according to the second aspect. In yet another embodiment, the method of inhibiting histone deacetylase in a cell comprises contacting the cell with an inhibiting effective amount of compound according to the first aspect and Embodiments A to GGG. In still another embodiment, the method of inhibiting histone deacetylase in a cell comprising contacting the cell with an inhibiting effective amount of a composition according to the second aspect.

For purposes of the present invention, the following definitions will be used (unless expressly stated otherwise).

As used herein, the terms “histone deacetylase” and “HDAC” are intended to refer to any one of a family of enzymes that remove acetyl groups from the ω-amino groups of lysine residues at the N-terminus of a histone. Unless otherwise indicated by context, the term “histone” is meant to refer to any histone protein, including H1, H2A, H2B, H3, H4, and H5, from any species. Preferred histone deacetylases include class I and class II enzymes. Other preferred histone deacetylases include class III enzymes. Preferably the histone deacetylase is a human HDAC, including, but not limited to, HDAC-1, HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7. In some other preferred embodiments, the histone deacetylase is derived from a protozoal or fungal source.

The terms “histone deacetylase inhibitor” and “inhibitor of histone deacetylase” are intended to mean a compound having a structure as defined herein, which is capable of interacting with a histone deacetylase and inhibiting its enzymatic activity.

The term “inhibiting histone deacetylase enzymatic activity” is intended to mean reducing the ability of a histone deacetylase to remove an acetyl group from a histone. The concentration of inhibitor which reduces the activity of a histone deacetylase to 50% of that of the uninhibited enzyme is determined as the IC₅₀ value.

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for 0, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetoaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” is intended to a mean straight and branched chain aliphatic group having from 1 to 12 carbon atoms, preferably 1-8 carbon atoms, and more preferably 1-6 carbon atoms, which is optionally substituted with one, two or three substituents. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” is intended to mean an unsaturated straight or branched chain aliphatic group with one or more carbon-carbon double bonds, having from 2 to 12 carbon atoms, preferably 2-8 carbon atoms, and more preferably 2-6 carbon atoms, which is optionally substituted with one, two or three substituents. Preferred alkenyl groups include, without limitation, ethenyl, propenyl, butenyl, pentenyl, and hexenyl.

The term “alkynyl” is intended to mean an unsaturated straight or branched chain aliphatic group with one or more carbon-carbon triple bonds, having from 2 to 12 carbon atoms, preferably 2-8 carbon atoms, and more preferably 2-6 carbon atoms, which is optionally substituted with one, two or three substituents. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” is intended to mean a saturated or unsaturated mono-, bi, tri- or poly-cyclic hydrocarbon group having about 3 to 15 carbons, preferably having 3 to 12 carbons, preferably 3 to 8 carbons, and more preferably 3 to 6 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” is intended to mean a saturated or unsaturated, straight or branched chain aliphatic group, as defined hereinabove, wherein one or more carbon atoms in the chain are replaced by a heteroatom selected from the group consisting of O, S, and N.

The term “aryl” is intended to mean a mono-, bi-, tri- or polycyclic C₆-C₁₄ aromatic moiety, preferably comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl.

The terms “aralkyl” or “arylalkyl” is intended to mean a group comprises an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The term “heterocyclyl” is intended to mean a group which is an optionally substituted mono-, bi-, tri- or polycyclic structure having from about 3 to 17, preferably about 3 to about 14 atoms, wherein one or more atoms are selected from the group consisting of N, O, and S. One ring of a bicyclic heterocycle or two rings of a tricyclic heterocycle may be aromatic, as in indan and 9,10-dihydro anthracene. The heterocyclic group is optionally substituted on carbon with oxo or another substituent. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean an optionally substituted mono-, bi-, tri- or polycyclic group having 5 to 14 ring atoms, preferably 5, 6, 9, or 10 ring atoms; having 6, 10, or 14 pi electrons shared in a cyclic array; and having, in addition to carbon atoms, between one or more heteroatoms selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Aromatic polycycles include, but are not limited to, naphthyl, and naphthyl substituted by one or more suitable substituents, including C₁-C₆alkyl, cycloalkylalkyl (e.g. cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and ORₐₐ, such as alkoxy, wherein Rₐₐ is selected from the group consisting of H, C₁-C₆alkyl, C₄-C₉cycloalkyl, C₄-C₉heterocycloalkyl, aryl, heteroaryl, arylalkyl, heteroarylalkyl and (CH₂)₀₋₆ZₐR_{bb}, wherein Zₐ is selected from the group consisting of O, NR_{cc}, S and S(O), and R_{bb} is selected from the group consisting of H, C₁-C₆alkyl, C₄-C₉cycloalkyl, C₄-C₉heterocycloalkyl, C₄-C₉heterocycloalkylalkyl, aryl, mixed aryl and non-aryl polycycle, heteroaryl, arylalkyl, (e.g. benzyl), and heteroarylalkyl (e.g. pyridylmethyl); and R_{cc} is selected from the group consisting of H, C₁-C₆alkyl, C₄-C₉cycloalkyl, C₄-C₉heterocycloalkyl, aryl, heteroaryl, arylalkyl (e.g. benzyl), heteroarylalkyl (e.g. pyridylmethyl) and amino acyl.

Non-aromatic polycycle substituents include, but are not limited to, bicyclic and tricyclic fused ring systems where each ring can be 4-9 membered and each ring can conatin zero, 1 or more double and/or triple bonds. Suitable examples of non-aromatic polycycles include, but are not limited to, decalin, octahydroindene, perhydrobenzocycloheptene and perhydrobenzo-[f]-azulene. Such substituents are themselves optionally substituted with for example, but not limited to, C₃-C₉cycloalkyl groups, such as cyclopropyl, cyclobutyl, cyclopentyl, cyclohexyl and the like. Unless otherwise noted, non-aromatic polycycle substituents include both unsubstituted cycloalkyl groups and cycloalkyl groups that are substituted by one or more suitable substituents, including but not limited to, C₁-C₆alkyl, halo, hydroxy, aminoalkyl, oxyalkyl, alkylamino and ORₐₐ, such as alkoxy. Preferred substituents for such cycloalkyl groups include halo, hydroxy, alkoxy, oxyalkyl, alkylamino and aminoalkyl.

Mixed aryl and non-aryl polycycle substituents include bicyclic and tricylic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane dihydroanthracene and 9H-fluorene. Such substituents are unsubstituted or substituted by nitro or as described above for non-aromatic polycycle substituents.

Polyheteroaryl substituents include bicyclic and tricyclic fused rings systems where each ring can independently be 5 or 6 membered and contain one or more heteroatom, for example, 1, 2, 3 or 4 heteroatoms, chosen from O, N or S such that the fused ring system is aromatic. Suitable examples or polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including but not limited to, straight and branched optionally substituted C₁-C₆alkyl, unsaturation (i.e., there are one or more double or triple C—C bonds), acyl, cycloalky, halo, oxyalkyl, alkylamino, aminoalkyl, acylamino and ORₐₐ, for example alkoxy, and a substituent of the formula —O—(CH₂CH═CH(CH₃)(CH₂))₁₋₃H. Examples of suitable straight and branched C₁-C₆alkyl substituents include but are not limited to methyl, ethyl, n-propyl, 2-propyl, n-butyl, sec-butyl, t-butyl and the like. Preferred substituents include halo, hydroxy, alkoxy, oxyalkyl, alkylamino and aminoalkyl. Nitrogen atoms are unsubstituted or substituted, for example by R_{cc}. Preferred substituents on such nitrogen atoms include H, C₁-C₄alkyl, acyl, aminoacyl and sulfonyl.

Non-aromatic polyheterocyclic substituents include but are not limited to bicyclic and tricyclic ring systems where each ring can be 4-9 membered, contain one or more heteratom, for example 1, 2, 3 or 4 heteratoms, chosen from O, N or S and contain zero, or one or more C—C double or triple bonds. Suitable examples of non-aromatic polyheterocycles include but are not limited to, hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydrop-1H-dicyclopenta[b,e]pyran. Unless otherwise noted, non-aromatic polyheterocyclic substituents are unsubstituted or substituted on a carbon atom by one or more substituents, including but not limited to straight and branched optionally substituted C₁-C₆alkyl, unsaturation (i.e., there are one or more double or triple C—C bonds), acyl, cycloalky, halo, oxyalkyl, alkylamino, aminoalkyl, acylamino and ORₐₐ, for example alkoxy. Examples of suitable straight and branched C₁-C₆alkyl substituents include but are not limited to methyl, ethyl, n-propyl, 2-propyl, n-butyl, sec-butyl, t-butyl and the like. Preferred substituents include halo, hydroxy, alkoxy, oxyalkyl, alkylamino and aminoalkyl. Nitrogen atoms are unsubstituted are substituted, for example, by R_{cc}. Preferred N substituents include H, C₁-C₄ alkyl, acyl, aminoacyl and sulfonyl.

Mixed aryl and non-aryl polyheterocycles substituents include but are not limited to bicyclic and tricyclic fused ring systems where each ring can be 4-9 membered, contain one or more heteroatom chosen from O, N or S and at least one of the rings must be aromatic. Suitable examples of mixed aryl and non-aryl polyheteorcycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydropyrido[2,3-b][1,4]diazepin-4-one, 1,2,3,4,6,11-hexhydro-benzo[b]pyrido[2,3-e][1,4]diazepine-5-one. Unless otherwise noted, mixed aryl and non-aryl polyheterocyclic substituents are unsubstituted or substituted on a carbon atom by one or more suitable substituents including but not limited to —N—OH, ═N—OH, optionally substituted alkyl unsaturation (i.e., there are one or more double or triple C—C bonds), acyl, cycloalky, halo, oxyalkyl, alkylamino, aminoalkyl, acylamino and ORₐₐ, for example alkoxy. Nitrogen atoms are unsubstituted or substituted, for example, by R_{cc}. Preferred N substituents include H, C₁₋₄alkyl, acyl aminoacyl and sulfonyl.

As employed herein, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—)nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl or any of these rings fused or spiro-fused to a cycloalkyl, heterocyclyl, or aryl, wherein each of the foregoing is further optionally substituted with one more moieties listed in (a), above; and
(c) —(CH₂)ₙ—NR₃OR₃₁, wherein n is from 0 (in which case the nitrogen is directly bonded to the moiety that is substituted) to 6, and R₃₀ and R₃, are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or

The term “halogen” or “halo” is intended to mean chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” is intended to mean a chemical moiety comprising one or more unpaired electrons.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluoro-3-propylphenyl. As another non-limiting example, substituted N-octyls include 2,4-dimethyl-5-ethyl-octyl and 3-cyclopentyl-octyl. Included within this definition are methylenes (—CH₂—) substituted with oxygen to form carbonyl —CO—).

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. Substituents on cyclic moieties also include 5-6 membered mono- and 9-14 membered bi-cyclic moieties attached to the parent cyclic moiety by a covalent bond to form a bi- or tri-cyclic bi-ring system. For example, an optionally substituted phenyl includes, but is not limited to, the following:

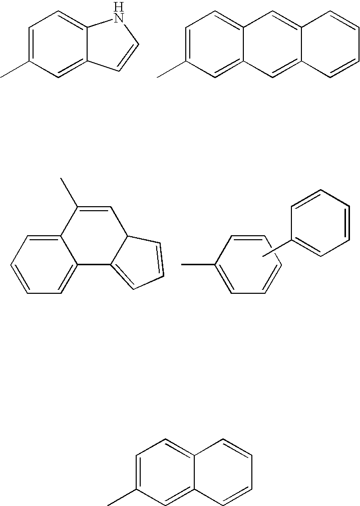

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

The term “protecting group” is intended to mean a group used in synthesis to temporarily mask the characteristic chemistry of a functional group because it interferes with another reaction. A good protecting group should be easy to put on, easy to remove and in high yielding reactions, and inert to the conditions of the reaction required. A protecting group or protective group is introduced into a molecule by chemical modification of a functional group in order to obtain chemoselectivity in a subsequent chemical reaction. One skilled in the art will recognize that during any of the processes for preparation of the compounds in the present invention, it may be necessary and/or desirable to protect sensitive or reactive groups on any of the molecules concerned. This may be achieved by means of conventional protecting groups, such as but not limited to Bn- (or —CH₂Ph), —CHPh₂, alloc (or CH₂═CH—CH₂—O—C(O)—), BOC—, -Cbz (or Z-), —F-moc, —C(O)—CF₃, N-Phthalimide, 1-Adoc-, TBDMS-, TBDPS-, TMS-, TIPS-, IPDMS-, —SiR₃, SEM-, t-Bu-, Tr-, THP- and Allyl-. These protecting groups may be removed at a convenient stage using methods known from the art.

Some compounds of the invention may have chiral centers and/or geometric isomeric centers (E- and Z-isomers), and it is to be understood that the invention encompasses all such optical, diastereoisomers and geometric isomers. The invention also comprises all tautomeric forms of the compounds disclosed herein.

The compounds of the invention may be administered as is or in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any carboxy group in the compounds of this invention.

An in vivo hydrolyzable ester of a compound of the invention containing a hydroxy group includes inorganic esters such as phosphate esters and a-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁₋₆-alkyl or N,N-di-C₁₋₆-alkyl amide such as N-methyl, N-ethyl, N-propyl, N,N-dimethyl, N-ethyl-N-methyl or N,N-diethyl amide.

The foregoing merely summarizes the one aspect and embodiments of the invention and is not intended to be limiting in nature. These aspects and embodiments are described more fully below.

Compounds

Some examples of the compounds according to the one aspect of the invention and Embodiments A to FFF are listed in the table below. These examples merely serve to exemplify some of the compounds of the one aspect of the invention and do not limit the scope of the invention.

TABLE 1a
2-(2-(6-(biphenyl-3-ylamino)-6-oxohexylamino)-2-oxoethylthio)acetic acid
N-(biphenyl-3-yl)-6-(2-(4-(methylthio)benzylthio)acetamido)hexanamide
methyl 3-(2-(6-(biphenyl-3-ylamino)-6-oxohexylamino)-2-oxoethylthio)-2-
hydroxypropanoate
methyl 4-(2-(6-(biphenyl-3-ylamino)-6-oxohexylamino)-2-oxoethylthio)butanoate
methyl 2-(2-(6-(biphenyl-3-ylamino)-6-oxohexylamino)-2-oxoethylthio)acetate
(S)-tert-butyl 6-(2-(benzyloxy)acetamido)-1-oxo-1-(quinolin-8-ylamino)hexan-2-
ylcarbamate
N-(biphenyl-3-yl)-6-(2-(2-oxo-2-(phenylamino)ethylthio)acetamido)hexanamide
2-(2-(6-(biphenyl-3-ylamino)-6-oxohexylamino)-2-oxoethylsulfinyl)acetic acid
Methyl 2-(2-(2-(biphenyl-3-ylamino)-2-oxoethylamino)-2-oxoethylsulfonyl)acetate
6-(2-(2-aminoethoxy)acetamido)-N-(biphenyl-3-yl)hexanamide
N-(biphenyl-3-yl)-6-(2-(4-fluorobenzyloxy)ethanethioamido)hexanamide
4-((2-(6-(biphenyl-3-ylamino)-6-oxohexylamino)-2-oxoethoxy)methyl)benzoic acid
N-(biphenyl-3-yl)-6-(2-(4-(hydroxymethyl)benzyloxy)acetamido)hexanamide
N-(biphenyl-3-yl)-6-(2-(4-cyanobenzyloxy)-acetamido)-hexanamide
N-(biphenyl-3-yl)-6-(2-(4-methylbenzyloxy)-acetamido)-hexanamide
N-(biphenyl-3-yl)-6-(2-(thiophen-2-ylmethoxy)-acetamido)-hexanamide
N-(biphenyl-3-yl)-6-(2-(thiophen-3-ylmethoxy)-acetamido)-hexanamide
N-(biphenyl-3-yl)-6-(2-(furan-3-ylmethoxy)-acetamido)-hexanamide
N-(biphenyl-3-yl)-6-(2-(4-bromobenzyloxy)-acetamido)-hexanamide
N-(biphenyl-3-yl)-6-(2-(naphthalen-1-ylmethoxy)-acetamido)-hexanamide
N-(biphenyl-3-yl)-6-(2-(2-oxo-2-(phenylamino)-ethylsulfinyl)-acetamido)-hexanamide
3-(2-(6-(biphenyl-3-ylamino)-6-oxohexylamino)-2-oxoethylthio)-propanoic acid
methyl 3-(2-(6-(biphenyl-3-ylamino)-6-oxohexylamino)-2-oxoethylsulfinyl)-propanoate
N-(biphenyl-3-yl)-6-(2-(3-hydroxypropylthio)-acetamido)-hexanamide
N-(biphenyl-3-yl)-6-(2-(2-hydroxyethylthio)-acetamido)-hexanamide
6-(2-(2-amino-2-oxoethylthio)-acetamido)-N-(biphenyl-3-yl)hexanamide
(R)-ethyl 2-amino-3-(2-(6-biphenyl-3-ylamino)-6-oxohexylamino)-2-oxoethylthio)-
propanoate
(R)-2-amino-3-(2-(6-biphenyl-3-ylamino)-6-oxohexylamino)-2-oxoethylthio)-propanoic
acid
6-(2-(2-aminoethylthio)-acetamido)-N-(biphenyl-3-yl)hexanamide
N-(biphenyl-3-yl)-6-(2-(4-hydroxyphenylthio)-acetamido)-hexanamide
methyl 2-(2-(6-(biphenyl-3-ylamino)-6-oxohexylamino)-2-oxoethylsulfinyl)-acetate
N-(biphenyl-3-yl)-6-(2-(3-oxo-3-(phenylamino)-propylthio)-acetamido)-hexanamide
N-(biphenyl-3-yl)-6-(2-(3-oxo-3-(phenylamino)-propylsulfinyl)-acetamido)-hexanamide
N-(biphenyl-3-yl)-6-(2-(3-hydroxyphenylthio)-acetamido)-hexanamide
6-(2-(4-aminophenylthio)-acetamido)-N-(biphenyl-3-yl)hexanamide
N-(biphenyl-3-yl)-6-(2-(4-fluorophenylthio)-acetamido)-hexanamide
N-(biphenyl-3-yl)-6-(2-(phenylthio)-acetamido)-hexanamide
N-(biphenyl-3-yl)-6-(2-(4-chlorophenylthio)-acetamido)-hexanamide
N-(biphenyl-3-yl)-6-(2-(4-bromophenylthio)-acetamido)-hexanamide
6-(2-(3-aminophenylthio)-acetamido)-N-(biphenyl-3-yl)hexanamide
N-(biphenyl-3-yl)-6-(2-(pyridin-4-ylthio)acetamido)-hexanamide
N-(biphenyl-3-yl)-6-(2-(thiophen-2-ylthio)acetamido)-hexanamide
N-(biphenyl-3-yl)-6-(2-(4-(methylthio)benzyloxy)acetamido)hexanamide)
N-(biphenyl-3-yl)-6-(2-(naphthalen-2-ylmethoxy)-acetamido)-hexanamide
N-(biphenyl-3-yl)-6-(2-(4-fluorobenzyloxy)-acetamido)-hexanamide
N-(biphenyl-3-yl)-6-(2-(4-chlorobenzyloxy)-acetamido)-hexanamide
N-(biphenyl-3-yl)-6-(2-(pyridin-4-ylmethoxy)-acetamido)-hexanamide
N-(biphenyl-3-yl)-6-(2-(pyridin-3-ylmethoxy)-acetamido)-hexanamide
N-(biphenyl-3-yl)-6-(2-(4-(methylsulfinyl)-benzyloxy)-acetamido)-hexanamide
6-(2-(benzylthio)acetamido)-N-(biphenyl-3-yl)hexanamide
6-(2-(benzylsulfinyl)acetamido)-N-(biphenyl-3-yl)hexanamide
N-(biphenyl-3-yl)-6-(4-(thiophen-2-yl)butanamido)-hexanamide
N-(biphenyl-3-yl)-6-(3-(4-fluorobenzylthio)propanamido)hexanamide
N-(biphenyl-3-yl)-6-(2-(4-fluorobenzyl-thio)acetamido)-hexanamide
N-(biphenyl-3-yl)-6-(2-(4-fluorobenzyl-sulfinyl)-acetamido)-hexanamide
2-(3-(6-(biphenyl-3-ylamino)-6-oxohexylamino)-3-oxopropylthio)-acetic acid
2-(3-(6-(biphenyl-3-ylamino)-6-oxohexylamino)-3-oxopropyl-sulfinyl)acetic acid
6-(2-(benzyloxy)acetamido)-N-(biphenyl-3-yl)hexanamide
(S)-2-amino-6-(2-(benzyloxy)acetamido)hexanoic acid
(S)-benzyl 6-(2-(benzyloxy)acetamido)-1-oxo-1-(quinolin-8-ylamino)hexan-2-
ylcarbamate
(S)-2-(2-oxo-2-(4-((3-oxo-2-(thiophen-2-ylmethyl)-3,4-dihydroquinoxalin-1(2H)-
yl)methyl)phenylamino)ethylthio)acetic acid
N-(biphenyl-3-yl)-6-(2-(2-(pyridin-2-yl)ethylthio)acetamido)hexanamide,
N-(biphenyl-3-yl)-6-(2-(2-(diethylamino)ethylthio)acetamido)hexanamide
4-(2-(6-(biphenyl-3-ylamino)-6-oxohexylamino)-2-oxoethylthio)butanoic acid
N-(biphenyl-3-yl)-6-(2-(4-(methylsulfinyl)benzylthio)acetamido)hexanamide
N-(biphenyl-3-yl)-6-(2-(2-(dimethylamino)ethylthio)-acetamido)-hexanamide
(S)-3-(2-(4-((2-benzyl-3-oxo-3,4-dihydroquinoxalin-1(2H)-yl)methyl)phenylamino)-2-
oxoethylthio)propanoic acid
(R)-3-(2-oxo-2-(4-((3-oxo-2-(thiophen-2-ylmethyl)-3,4-dihydroquinoxalin-1(2H)-
yl)methyl)-phenylamino-)ethylthio)-propanoic acid
3-(2-(6-methoxybenzo[d]thiazol-2-ylamino)-2-oxoethylthio)propanoic acid
4-(6-(biphenyl-3-ylamino)-6-oxohexylamino)-2-hydroxy-4-oxobutanoic acid
3-(2-(6-(biphenyl-3-ylamino)-6-oxohexylamino)-2-oxoethylthio)-2-hydroxypropanoic acid
N-(4-(((2-(1H-indol-3-yl)ethyl)(2-hydroxyethyl)amino)methyl)benzyl)-2-(3-
hydroxypropylthio)acetamide
(R)-N-(4-((2-((1H-indol-3-yl)methyl)-3-oxo-3,4-dihydroquinoxalin-1(2H)-
yl)methyl)phenyl)-2-(2-(dimethylamino)ethylthio)acetamide
3-(2-(benzo[d]thiazol-2-ylamino)-2-oxoethylthio)propanoic acid
3-(2-(6-fluorobenzo[d]-thiazol-2-ylamino)-2-oxoethylthio)-propanoic acid
3-({2-[(6-nitro-1,3-benzothiazol-2-yl)amino]-2-oxoethyl}thio)propanoic acid
3-(2-(6-nitrobenzo[d]-thiazol-2-ylamino)-2-oxoethylthio)-propanoic acid
3-(2-oxo-2-(6-oxo-6-(3-(pyridin-3-yl)phenylamino)hexylamino)ethylthio)-propanoic acid
N-biphenyl-3-yl-6-({[(4-fluorobenzyl)oxy]acetyl}amino)hexanamide

Synthetic Schemes and Experimental Procedures

The compounds of the invention can be prepared according to the reaction schemes for the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

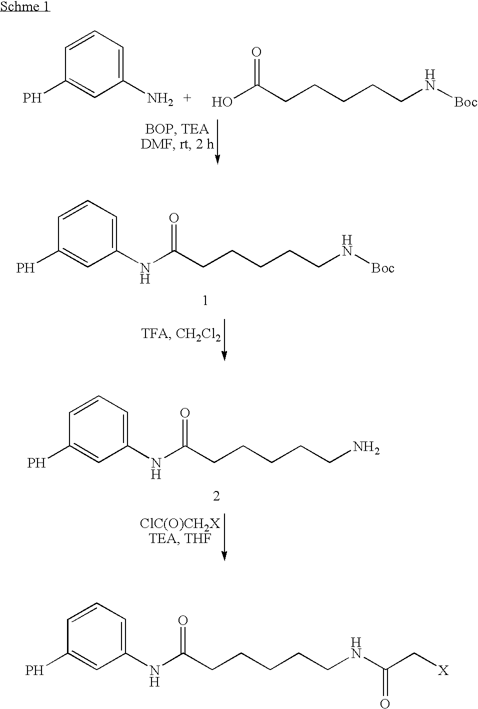

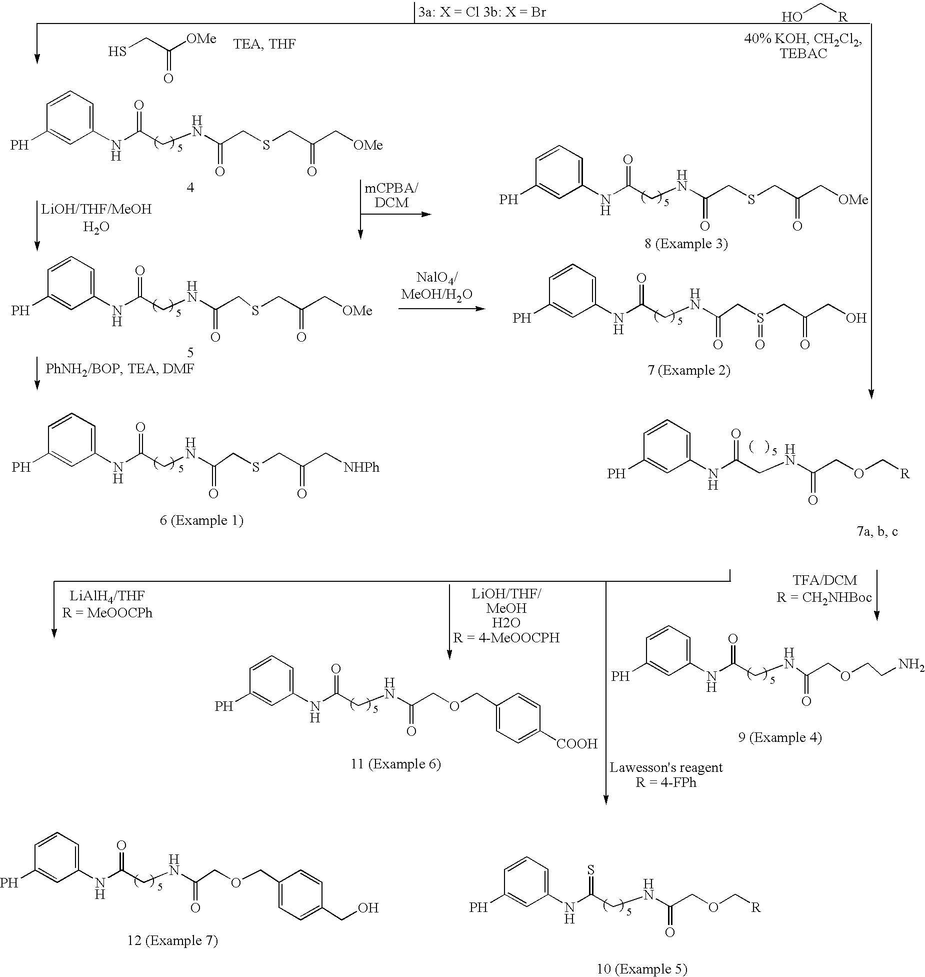

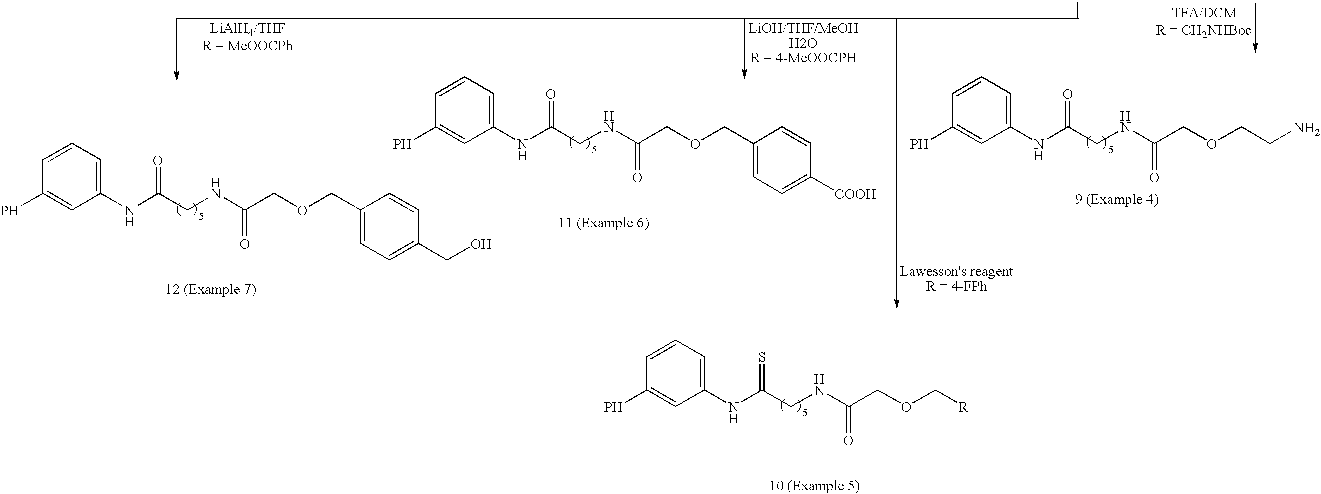

### EXAMPLE 1

### N-(biphenyl-3-yl)-6-(2-(2-oxo-2-(phenylamino)ethylthio)acetamido)hexanamide (6)

### Step 1: tert-butyl 6-(biphenyl-3-ylamino)-6-oxohexylcarbamate (1)

N-Boc-caproic acid (1.1 g, 4.77 mmol), 3-phenyl aniline (806 mg, 4.77 mmol) and BOP (2.1 g, 4.77 mmol) were dissolved in DMF (10 mL). Triethylamine (11.92 mmol, 1.66 mL) was added and the reaction was stirred for 3 hours at room temperature. The reaction was then quenched with water and extracted with ethyl acetate. The organic extract was dried (Na₂SO₄), filtered, and evaporated. The residue was purified by silica gel column chromatography with gradient of EtOAc (25-100%) in Hexane to afford 1 (1.65 g, 91%) as a beige solid. LRMS (ESI): (calc.) 382.2; (found) 383.3 (MH)⁺.

### Step 2: 6-amino-N-(biphenyl-3-yl)hexanamide (2)

To a solution of 1 (1.23 g, 3.22 mmol) in DCM (40 mL) was added TFA (6 mL). The mixture was stirred for 2 hours. The reaction was basified with NaHCO₃ (ss) and extracted with ethyl acetate. The organic layer was dried (Na₂SO₄), filtered, and evaporated to afford 2 (0.90 g, 98%) as viscous colorless oil. LRMS (ESI): (calc.) 282.5; (found) 283.0 (MH)⁺.

### Step 3: N-(biphenyl-3-yl)-6-(2-chloroacetamido)hexanamide (3a), N-(biphenyl-3-yl)-6-(2-bromoacetamido)hexanamide (3b)

Compound 3a: To a solution of 2 (1.41 g, 5.00 mmol) in DCM (10 mL) was added triethylamine (1.00 mL, 7.17 mmol) and chloroacetyl chloride (0.40 mL, 5.00 mmol). The resulting mixture was stirred for 5 min at room temperature, and then concentrated under reduced pressure. The crude material was dissolved in EtOAc, washed with NaHCO₃ (ss), water and brine. The organic layer was dried (Na₂SO₄), filtered, and evaporated to give a brown oil which was purified by silica gel column chromatography with EtOAc/Hexanes/MeOH (10:9:1) to afford 3a (1.06 g, 59% after 3 steps) as a white solid. ¹H NMR: (MeOH-d4) δ (ppm): 7.84-7.83 (m, 1H), 7.60-7.58 (m, 2H), 7.53-7.50 (m, 1H), 7.43-7.31 (m, 5H), 4.00 (s, 2H), 3.25 (t, J=7.2 Hz, 2H), 2.42 (t, J=7.2 Hz, 2H), 1.80-1.72 (m, 2H), 1.64-1.57 (m, 2H), 1.48-1.42 (m, 2H); LRMS (ESI): (found) 359.1: 361.1 (r:3:1) (MH)⁺.

Compound 3b: To a solution of 2 (4.83 g, 17.1 mmol) in THF (50 mL) was added bromoacetyl chloride (1.43 mL, 17.1 mmol) and triethylamine (7.15 mL, 51.3 mmol). The mixture was stirred for 10 min prior to extraction from brine with EtOAc, dried (Na₂SO₄), filtered, and evaporated. The residue was purified by silica gel column chromatography with gradient of EtOAc (25-100%) in Hexane to afford 3b (2.16 g, 31%) as a light pink solid. ¹H NMR: (DMSO-d₆) δ (ppm): 10.00 (s,1H), 8.29 (m,1H), 7.94 (s,1H), 7.61 (m,3H), 7.49 (t, J=7.8 Hz, 2H), 7.40 (t, J=7.8 Hz, 2H), 7.33 (d, J=7.8 Hz, 1H), 3.38 (s,2H), 3.10 (q, J=13.2,6.6 Hz, 2H), 2.36 (t, J=7.4 Hz, 2H), 1.70-1.60 (m,2H), 1.54-1.44 (m,2H), 1.40-1.30 (m,2H). LRMS (ESI): (found) 403.2: 405.1 (r:1:1) (MH)⁺.

### Step 4: methyl 2-(2-(6-(biphenyl-3-ylamino)-6-oxohexylamino)-2-oxoethylthio)acetate (4)

To a solution of 3b (270 mg, 0.67 mmol) in THF (10 mL) was added methyl thioglycolate (0.67 mmol, 0.061 mL), and triethylamine (1.68 mmol, 0.24 mL). The reaction was stirred for 15 hours at room temperature (some related examples heat at reflux) then quenched with water and extracted with ethyl acetate. The organic extract was dried (Na₂SO₄), filtered, and evaporated. The residue was purified by silica gel column chromatography with gradient of EtOAc (25-50%) in Hexane to afford 4 (230 mg, 81%) as a white solid. ¹H NMR: (DMSO-d₆) δ (ppm): 9.94 (s, 1H), 7.98 (t, J=5.7 Hz, 1H), 7.90 (s, 1H), 7.59-7.54 (m, 3H), 7.45 (t, J=7.2 Hz, 2H), 7.35 (t, J=6.5 Hz, 2H), 7.30-7.27 (m, 1H), 3.62 (s, 3H), 3.43 (s, 2H), 3.18 (s, 2H), 3.05 (q, J=6.8 Hz, 2H), 2.32 (t, J=7.2 Hz, 2H), 1.62-1.58 (m, 2H), 1.45-1.39 (m, 2H), 1.34-1.28 (m, 2H). LRMS (ESI): (calc.) 428.2; (found) 429.2 (MH)⁺.

### Step 5: 2-(2-(6-(biphenyl-3-ylamino)-6-oxohexylamino)-2-oxoethylthio)acetic acid (5)

To a solution of methyl ester 4 (210 mg, 0.49 mmol) in THF:methanol:water solvent mixture (5:1:1), was added lithium hydroxide monohydrate (103 mg, 2.45 mmol) and heated at 60° C. for 1 hour. The reaction was acidified with 1 M HCl solution and extracted with ethyl acetate. The organic extract was dried (Na₂SO₄), filtered, and evaporated to afford 5 (130 mg, 65%) as a white solid. ¹H NMR: (DMSO-d₆) δ (ppm): 9.95 (s, 1H), 8.01 (t, J=5.3 Hz, 1H), 7.90 (s, 1H), 7.59-7.54 (m, 3H), 7.45 (t, J=7.6 Hz, 2H), 7.35 (t, J=7.8 Hz, 2H), 7.30-7.27 (m, 1H), 3.33 (s, 2H), 3.18 (s, 2H), 3.05 (q, J=6.7 Hz, 2H), 2.32 (t, J=7.2 Hz, 2H), 1.64-1.57 (m, 2H), 1.47-1.40 (m, 2H), 1.35-1.28 (m, 2H). LRMS (ESI): (calc) 414.2; (found) 415.4(MH)⁺.

### Step 6: N-(biphenyl-3-yl)-6-(2-(2-oxo-2-(phenylamino)ethylthio)acetamido)hexanamide (6)

Following the same procedure as described for compound 1 (scheme 1, example 1) but substituting acid 5 for N-Boc-caproic acid and aniline for 3-phenyl aniline to afford 6 (70 mg, 84%) as a white solid. ¹H NMR: (DMSO-d₆) δ (ppm): 10.11 (s, 1H), 9.94 (s, 1H), 8.04 (t, J=5.4 Hz, 1H), 7.90 (s, 1H), 7.58-7.54 (m, 5H), 7.45 (t, J=7.2 Hz, 2H), 7.35 (t, J=7.8 Hz, 2H), 7.30-7.25 (m, 3H), 7.28 (t, J=7.4 Hz, 2H), 3.42 (s, 2H), 3.27 (s, 2H), 3.06 (q, J=6.6 Hz, 2H), 2.31 (t, J=7.2 Hz, 2H), 1.63-1.56 (m, 2H), 1.47-1.40 (m, 2H), 1.34-1.27 (m, 2H). LRMS (ESI): (calc) 505.2; (found) 506.5 (MH)⁺.

### EXAMPLE 2

### 2-(2-(6-(biphenyl-3-ylamino)-6-oxohexylamino)-2-oxoethylsulfinyl)acetic acid (7)

### Step 6: 2-(2-(6-(biphenyl-3-ylamino)-6-oxohexylamino)-2-oxoethylsulfinyl)acetic acid (7)

To a stirred solution of 5 (30 mg, 0.061 mmol) (see step 1-5 example 1, scheme 1 for preparation) in methanol:water solution (2:1) was added sodium periodate (100 mg, 0.42 mmol). The mixture was stirred at room temperature for a week. After water was added to the reaction and extracted with ethyl acetate. The organic extract was dried (Na₂SO₄), filtered, and evaporated. The residue was purified by trituration with EtOAc:MeOH (10:1) to afford 7 (24 mg, 78%) as a beige solid. ¹H NMR: (DMSO-d₆) δ (ppm): 10.33 (s, 1H), 9.94 (s, 1H), 8.31-8.29 (m, 1H), 7.90 (s, 1H), 7.59-7.53 (m, 5H), 7.45 (t, J=7.5 Hz, 2H), 7.37-7.28 (m, 5H), 7.06 (t, J=7.5 Hz, 1H), 3.83 (dd, J=32.7, 13.3 Hz, 1H), 3.84 (d, J=12.5 Hz, 2H), 3.12 (q, J=6.3 Hz, 2H), 2.32 (t, J=7.2 Hz, 2H), 1.63-1.59 (m, 2H), 1.48-1.44 (m, 2H), 1.34-1.32 (m, 2H). LRMS (ESI): (calc) 430.1; (found) 431.0 (MH)⁺.

### EXAMPLE 3

### Methyl 2-(2-(2-(biphenyl-3-ylamino)-2-oxoethylamino)-2-oxoethylsulfonyl)acetate (8)

### Step 5: Methyl 2-(2-(2-(biphenyl-3-ylamino)-2-oxoethylamino)-2-oxoethylsulfonyl)-acetate (8)

To a solution of 4 (as described in example 1, scheme 1, steps 1-4 for preparation) in DCM (6 mL) was added mCPBA (0.034 g, 0.196 mmol). The resulting solution was stirred at room temperature for 2 h and then evaporated. The residue was purified by silica gel column chromatography with gradient of MeOH (0-20%) in EtOAc to afford 8 (20 mg, 44%) as a white crystalline solid. (DMSO-d6) δ (ppm) ¹H, 10.00 (s,1H), 8.44 (s,1H), 7.96 (s,1H), 7.65-7.57 (m,3H), 7.49 (t, J=7.6 Hz, 2H), 7.43-7.36 (m,2H), 7.35-7.31 (m,1H), 4.59 (s,2H), 4.23 (s,2H), 3.74 (s,3H), 3.20-3.11 (m,2H), 2.37 (t, J=7.2 Hz, 2H), 1.71-1.60 (m,2H), 1.55-1.45 (m,2H), 1.43-1.32 (m,2H) LRMS (ESI): (calc.) 460.5; (found) 461.3 (MH)⁺.

### EXAMPLE 4

### 6-(2-(2-aminoethoxy)acetamido)-N-(biphenyl-3-yl)hexanamide (9)

### Step 4: tert-butyl 2-(2-(6-(biphenyl-3-ylamino)-6-oxohexylamino)-2-oxoethoxy)ethylcarbamate (7a)

To a solution of 3b (as described in example 1, scheme 1, steps 1-3 for preparation) (0.150 g, 0.372 mmol) in DCM (3 mL) was added tert-butyl-2-hydroxyethylcarbamate (0.575 mL, 3.72 mmol), benzyltriethylammonium chloride (0.169 g, 0.744 mmol), and 40% (w/v) aqueous KOH (3 mL). The resulting solution was stirred at room temperature for 2 h prior to extraction from brine with EtOAc. The residue was purified by silica gel column chromatography with gradient of EtOAc (25-100%) in hexane to afford 7a (110 mg, 61%) as a light yellow foam. LRMS (ESI): (calc.) 483.6; (found) 484.3 (MH)⁺.

### Step 5: 6-(2-(2-aminoethoxy)acetamido)-N-(biphenyl-3-yl)hexanamide (9)

To a solution of 7a (0.100 g, 0.207 mmol) in DCM (5 mL) was added TFA (0.5 mL). The mixture was stirred for 2 h at room temperature. The reaction was basified with aqueous NaOH and extracted with ethyl acetate. The organic extract was dried (Na₂SO₄), filtered, and evaporated to afford 9 (75 mg, 95%) as a white foam. LRMS (ESI): (calc.) 383.5; (found.) 384.2 (MH)⁺.

### EXAMPLE 5

### N-(biphenyl-3-yl)-6-(2-(4-fluorobenzyloxy)ethanethioamido)hexanamide (10)

### Step 4: N-(biphenyl-3-yl)-6-(2-(4-fluorobenzyloxy)acetamido)hexanamide (7b)

Following the same procedure as described for compound 7a (scheme 1, example 4) but substituting (4-fluorophenyl)methanol for tert-butyl-2-hydroxyethylcarbamate to afford 7b (160 mg, 91%) as a white solid. (DMSO-d6) δ (ppm) ¹H: 9.95 (s, 1H), 7.90 (s, 1H), 7.80 (t, J=5.5 Hz, 1H), 7.59-7.54 (m, 3H), 7.47-7.28 (m, 7H), 7.18-7.13 (m, 2H), 4.49 (s, 2H), 3.86 (s, 2H), 3.10 (q, J=6.7 Hz, 2H), 2.33 (t, J=7.4 Hz, 2H), 1.65-1.57 (m, 2H), 1.48-1.43 (m, 2H), 1.33-1.27 (m, 2H). MS: 448.2 (calc) 449.2 (found). LRMS (ESI): (calc) 448.2; (found) 449.2 (MH)⁺.

### Step 5: N-(biphenyl-3-yl)-6-(2-(4-fluorobenzyloxy)ethanethioamido)hexanamide (10)

To a solution of 7b (0.026 g, 0.058 mmol) in THF (3 mL) was added Lawesson's reagent (0.059 g, 0.145 mmol). The mixture was heated to 80° C. for 10 min prior to extraction from brine with EtOAc. The organic layer was dried (Na₂SO₄), filtered, and evaporated. The residue was purified by silica gel column chromatography with gradient of EtOAc (25-100%) in Hexane to afford 10 (21 mg, 75%) as a light yellow oil. (DMSO-d6) δ (ppm) ¹H, 9.58 (m,1H), 8.21 (s,1H), 7.78-7.68 (m,3H), 7.65 (d, J=7.0 Hz, 2H), 7.56-7.38 (m,7H), 7.20 (t, J=9.0 Hz, 2H), 4.56 (s,2H), 4.28 (s,2H), 3.65 (q, J=13.3, 6.7 Hz, 2H), 2.81 (t, J=7.4 Hz, 2H), 1.90-1.80 (m,2H), 1.74-1.63 (m,2H), 1.46-1.36 (m,2H). LRMS (ESI): (calc.) 480.7; (found) 481.4 (MH)⁺.

### EXAMPLE 6

### 4-((2-(6-(biphenyl-3-ylamino)-6-oxohexylamino)-2-oxoethoxy)methyl)benzoic acid (11)

### Step 4: methyl 4-((2-(6-(biphenyl-3-ylamino)-6-oxohexylamino)-2-oxoethoxy)methyl)benzoate (7c)

Following the same procedure as described for compound 7a (scheme 1 example 4) but substituting methyl 4-(hydroxymethyl)benzoate for tert-butyl-2-hydroxyethylcarbamate to 7c (43 mg, 18%) as a white solid (DMSO-d6) δ (ppm) ¹H: 9.98 (s,1H), 7.95 (m,3H), 7.89 (t, J=5.6 Hz, 1H), 7.60 (m,3H), 7.53 (d, J=8.0 Hz, 2H), 7.49 (t, J=7.8 Hz, 2H), 7.39 (t, J=7.8 Hz, 2H), 7.33 (d, J=7.6 Hz, 1H), 4.64 (s,2H), 3.95 (s,2H), 3.88 (s,3H), 3.14 (q, J=13.0,6.5 Hz, 2H), 2.36 (t, J=7.0 Hz, 2H), 1.70-1.60 (m,2H), 1.56-1.46 (m,2H), 1.40-1.30 (m,2H). LRMS (ESI): (calc) 488.6; (found) 489.1 (MH)⁺.

### Step 5: 4-((2-(6-(biphenyl-3-ylamino)-6-oxohexylamino)-2-oxoethoxy)methyl)benzoic acid (11)

Following the same procedure as described for compound 5 (step 5, scheme 1, example 1) but substituting 7c for 4 to afford 11 (43 mg, 18%) as a white solid. (DMSO-d6) δ (ppm) ¹H, 10.18 (s,1H), 7.97 (s,1H), 7.94 (d, J=7.8 Hz, 2H), 7.82 (t, J=5.7 Hz, 1H), 7.61 (m,3H), 7.48 (t, J=7.8 Hz, 2H), 7.44-7.34 (m,4H), 7.32 (d, J=7.6 Hz, 1H), 4.58 (s,2H), 3.91 (s,2H), 3.13 (q, J=13.2,6.6 Hz, 2H), 2.36 (t, J=7.3 Hz, 2H), 1.68-1.58 (m,2H), 1.52-1.42 (m,2H), 1.36-1.24 (m,2H). LRMS (ESI): (calc.) 474.5; (found) 475.3 (MH)⁺.

### EXAMPLE 7

### N-(biphenyl-3-yl)-6-(2-(4-(hydroxymethyl)benzyloxy)acetamido)hexanamide (12)

### Step 5: N-(biphenyl-3-yl)-6-(2-(4-(hydroxymethyl)benzyloxy)acetamido)hexanamide (12)

To a solution of 7c (as described in example 6, scheme 1, steps 1-4 for preparation) (0.040 g, 0.082 mmol) in THF (2 mL) was added LiAlH₄ (0.006 g, 0.164 mmol). The resulting solution was stirred at room temperature for 5 min prior to extraction with EtOAc. The organic layer was dried (Na₂SO₄), filtered, and evaporated. The residue was purified by trituration with EtOAc in hexanes to afford 12 (20 mg, 54%) as a light yellow solid. (DMSO-d6) δ (ppm) ¹H: 9.98 (s,1H), 7.94 (s,1H), 7.81 (t, J=5.7 Hz, 1H), 7.64-7.57 (m,3H), 7.48 (t, J=7.8 Hz, 2H), 7.39 (t, J=7.8 Hz, 2H), 7.35-7.30 (m,5H), 4.52 (s,2H), 4.51 (s,2H), 3.87 (s,2H), 3.14 (q, J=13.3,6.6 Hz, 2H), 2.36 (t, J=7.4 Hz, 2H), 1.70-1.60 (m,2H), 1.54-1.45 (m,2H), 1.38-1.26 (m,2H). LRMS (ESI): (calc.) 460.6; (found) 461.2 (MH)⁺.

### EXAMPLES 8-25

Example 8-25 describe the preparation of compound 13-30 using the same procedures as described Example 1-6. Characterization data are presented in a Table 1.

TABLE 1

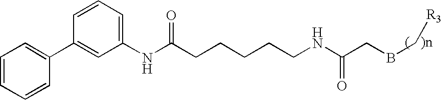

| Ex | Cpd | n | B | R₃ | Name | Characterization | Scheme |
|---|---|---|---|---|---|---|---|
| 8 | 13 | 1 | O | 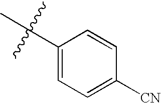 | N-(biphenyl-3- yl)-6-(2-(4- cyanobenzyloxy)- acetamido)- hexanamide | (DMSO-d6) δ (ppm) ¹H: 9.94 (s, 1H), 7.90-7.85 (m, 2H), 7.79 (d, J=8.2 Hz, 2H), 7.58-7.53 (m, 5H), 7.44 (t, J=7.2 Hz, 2H), 7.34 (t, J=7.6 Hz, 2H), 7.29-7.27 (m, 1H), 4.60 (s, 2H), 3.91 (s, 2H), 3.10 (q, J=6.7 Hz, 2H), 2.32 (t, J=7.4 Hz, 2H), 1.62-1.56 (m, 2H), 1.48-1.42 (m, 2H), 1.33-1.27 | |
| # (m, 2H). LRMS (ESI): (calc) 455.2; (found) 456.3 (MH)⁺. | 1 Step 1-4 Cpd 7c Ex 5 | | | | | | |
| 9 | 14 | 1 | O | 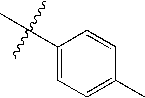 | N-(biphenyl-3- yl)-6-(2-(4- methylbenzyloxy)- acetamido)- hexanamide | (DMSO-d6) δ (ppm) ¹H: 9.95 (s, 1H), 7.90 (s, 1H), 7.75 (t, J=6.2 Hz, 1H), 7.59-7.54 (m, 3H), 7.45 (t, J=7.2 Hz, 2H), 7.35 (t, J=7.8 Hz, 2H), 7.30-7.27 (m, 1H), 7.22 (d, J=8.0 Hz, 2H), 7.13 (d, J=7.8 Hz, 2H), 4.45 (s, 2H), 3.82 (s, 2H), 3.09 (q, J=6.6 Hz, 2H), 2.32 (t, J=7.2 Hz, 2H), 2.28 (s, | |
| # 3H), 1.62-1.57 (m, 2H), 1.47-1.42 (m, 2H), 1.33-1.27 (m, 2H). LRMS (ESI): (calc) 444.3; (found) 445.3 (MH)⁺. | 1 Step 1-4 Cpd 7c Ex 5 | | | | | | |
| 10 | 15 | 1 | O | 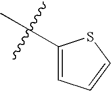 | N-(biphenyl-3- yL)-6-(2- (thiophen-2- ylmethoxy)- acetamido)- hexanamide | (DMSO-d6) δ (ppm) ¹H: 9.98 (br s, 1H), 7.94 (br s, 1H), 7.79 (br t, 1H), 7.64-7.57 (m, 3H), 7.54 (d, J=4.9 Hz, 1H), 7.49 (t, J=7.6 Hz, 2H), 7.39 (t, J=7.8 Hz, 2H), 7.34 (d, J=7.4 Hz, 1H), 7.11 (m, 1H), 7.03 (m, 1H), 4.72 (s, 2H), 3.88 (s, 2H), 3.12 (q, J=13.0, 6.3 Hz, 2H), 2.36 (t, J=7.4 | |
| # Hz, 2H), 1.70-1.60 (m, 2H), 1.54-1.44 (m, 2H), 1.38-1.28 (m, 2H). LRMS (ESI): (calc) 436.6; (found) 437.2 (MH)⁺. | 1 Step 1-4 Cpd 7c Ex 5 | | | | | | |
| 11 | 16 | 1 | O | 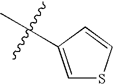 | N-(biphenyl-3- yL)-6-(2- (thiophen-3- ylmethoxy)- acetamido)- hexanamide | (DMSO-d6) δ (ppm) ¹H: 9.98 (s, 1H), 7.94 (s, 1H), 7.79 (t, J=5.7 Hz, 1H), 7.64-7.55 (m, 3H), 7.53 (m, 1H), 7.49 (m, 3H), 7.39 (t, J=7.8 Hz, 2H), 7.33 (d, J=7.6 Hz, 1H), 7.13 (d, J=4.8 Hz, 1H), 4.54 (s, 2H), 3.87 (s, 2H), 3.13 (q, J=13.0, 6.6 Hz, 2H), 2.36 (t, J=7.4 Hz, 2H), 1.70-1.59 (m, 2H), | |
| # 1.54-1.44 (m, 2H), 1.38-1.26 (m, 2H). LRMS (ESI): (calc) 436.6; (found) 437.2 (MH)⁺. 1 Step 1-4 Cpd 7c Ex 5 | | | | | | | |
| 12 | 17 | 1 | O | 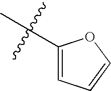 | N-(biphenyl-3- yl)-6-(2-(furan-3- ylmethoxy)- acetamido)- hexanamide | (DMSO-d6) δ (ppm) ¹H: 9.98 (br s, 1H), 7.94 (br s, 1H), 7.76 (t, J=5.7 Hz, 1H), 7.71 (s, 1H), 7.65 (t, J=1.6 Hz, 1H), 7.64-7.56 (m, 3H), 7.49 (t, J=7.8 Hz, 2H), 7.39 (t, J=7.6 Hz, 2H), 7.35 (d, J=7.6 Hz, 1H), 6.54 (s, 1H), 4.41 (s, 2H), 3.85 (s, 2H), 3.13 (q, J=13.3, 6.8 Hz, 2H), 2.36 (t, J= | |
| # 7.2 Hz, 2H), 1.70-1.60 (m, 2H), 1.54-1.44 (m, 2H), 1.38-1.28 (m, 2H). LRMS (ESI): (calc) 420.5; (found) 421.3 (MH)⁺. | 1 Step 1-4 Cpd 7c Ex 5 | | | | | | |
| 13 | 18 | 1 | O | 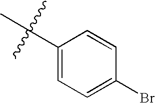 | N-(biphenyl-3- yl)-6-(2-(4- bromobenzyloxy)- acetamido)- hexanamide | (DMSO-d6) δ (ppm) ¹H: 9.99 (br s, 1H), 7.94 (br s, 1H), 7.85 (t, J=5.7 Hz, 1H), 7.64-7.58 (m, 2H), 7.58-7.54 (m, 2H), 7.49 (t, J=7.8 Hz, 2H), 7.42-7.30 (m, 5H), 4.52 (s, 2H), 3.38 (s, 2H), 3.14 (q, J=13.1, 6.5 Hz, 2H), 2.36 (t, J=7.2 Hz, 2H), 1.70-1.60 (m, 2H), 1.54-1.44 (m, 2H), 1.38-1.28 | |
| # (m, 2H). LRMS (ESI): (calc) 509.4; (found) 511.2 (MH)⁺. | 1 Step 1-4 Cpd 7c Ex 5 | | | | | | |
| 14 | 19 | 1 | O | 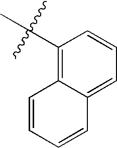 | N-(biphenyl-3- yl)-6-(2- (naphthalen-1- ylmethoxy)- acetamido)- hexanamide | (DMSO-d6) δ (ppm) ¹H: 10.00 (s, 1H), 8.17 (d, J=7.8 Hz, 1H), 7.95 (d, J=8.0 Hz, 2H), 7.90 (d, J=8.0 Hz, 1H), 7.80 (t, J=5.7 Hz, 1H), 7.65-7.55 (m, 5H), 7.54-7.45 (m, 3H), 7.42-7.35 (m, 2H), 7.32 (d, J=7.6 Hz, 1H), 5.01 (s, 2H), 4.00 (s, 2H), 3.15 (q, J=12.5, 6.3 Hz, 2H), 2.37 (t, J= | |
| # 7.2 Hz, 2H), 1.71-1.60 (m, 2H), 1.55-1.44 (m, 2H), 1.38-1.28 (m, 2H). LRMS (ESI): (calc) 480.6; (found) 481.3 (MH)⁺. | 1 Step 1-4 Cpd 7c Ex 5 | | | | | | |
| 15 | 20 | 1 | SO | 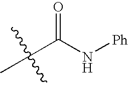 | N-(biphenyl-3- yl)-6-(2-(2-oxo-2- (phenylamino)- ethylsulfinyl)- acetamido)- hexanamide | (DMSO-d6) δ (ppm) ¹H: 10.33 (s, 1H), 9.94 (s, 1H), 8.31-8.29 (m, 1H), 7.90 (s, 1H), 7.59-7.53 (m, 5H), 7.45 (t, J=7.5 Hz, 2H), 7.37-7.28 (m, 5H), 7.06 (t, J=7.5 Hz, 1H), 3.83 (dd, J=32.7, 13.3 Hz, 1H), 3.84 (d, J=12.5 Hz, 2H), 3.12 (q, J=6.3 Hz, 2H), 2.32 (t, J=7.2 Hz, | |
| # 2H), 1.63-1.59 (m, 2H), 1.48-1.44 (m, 2H), 1.34-1.32 (m, 2H). LRMS (ESI): (calc) 505.2; (found) 506.5 (MH)⁺. | 1 Step 1-6 Ex 1 Step 6 Ex 2 | | | | | | |
| 16 | 21 | 2 | S | 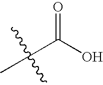 | 3-(2-(6- (biphenyl-3- ylamino)-6- oxohexylamino)- 2-oxoethylthio)- propanoic acid | (DMSO-d6) δ (ppm) ¹H: 12.28 (br s, 1H), 9.99 (s, 1H), 8.01 (m, 1H), 7.94 (s, 1H), 7.57-7.65 (m, 3H), 7.49 (t, J=7.8 Hz, 2H), 7.36-7.43 (m, 2H), 7.31-7.36 (m, 1H), 3.12 (s, 2H), 3.14-3.06 (m, 2H), 2.75 (t, J=7.0 Hz, 2H), 2.52-2.58 (m, 2H), 2.36 (t, J=7.6 Hz, 2H), 1.60-1.70 (m, 2H), | |
| # 1.42-1.53 (m, 2H), 1.31-1.40 (m, 2H) LRMS (ESI): (calc) 428.5; (found) 429.5 (MH)⁺. | 1 Step 1-5 Ex 1 | | | | | | |
| 17 | 22 | 2 | SO | 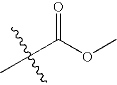 | methyl 3-(2-(6- (biphenyl-3- ylamino)-6- oxohexylamino)- 2- oxoethylsulfinyl)- propanoate | (DMSO-d6) δ (ppm) ¹H: 9.99 (s, 1H), 8.26 (m, 1H), 7.94 (s, 1H), 7.56-7.64 (m, 3H), 7.49 (t, J=7.8 Hz, 2H), 7.36-7.43 (m, 2H), 7.32 (m, 1H), 3.73 (d, J=12.9 Hz, 1H), 3.66 (s, 3H), 3.58 (d, J=12.9 Hz, 1H), 3.09-3.20 (m, 3H), 2.95-3.03 (m, 1H), 2.77 (t, J=7.4 Hz, 2H), 2.36 | |
| # (t, J=7.4 Hz, 2H), 1.60-1.70 (m, 2H), 1.43-1.54 (m, 2H), 1.31-1.42 (m, 2H) LRMS (ESI): (calc) 458.6; (found) 458.9 (MH)⁺. | 1 Step 1-4 Ex 1 Step 5 Ex 2 | | | | | | |
| 18 | 23 | 3 | S | —OH | N-(biphenyl-3- yl)-6-(2-(3- hydroxypropyl- thio)-acetamido)- hexanamide | (DMSO-d6) δ (ppm) ¹H: 10.00 (s, 1H), 7.97 (m, 2H), 7.56-7.66 (m, 3H), 7.45-7.53 (m, 2H), 7.30-7.44 (m, 3H), 4.54 (m, 1H), 3.53-3.44 (m, 2H), 3.19-3.02 (m, 4H), 2.58-2.68 (m, 2H), 2.30-2.44 (m, 2H), 1.56-1.78 (m, 4H), 1.42-1.56 (m, 2H), 1.30-1.40 (m, 2H) LRMS (ESI): (calc) 414.6; (found) | |
| # 415.2 (MH)⁺. | 1 Step 1-4 Ex 1 Step 5 Ex 2 Step 5 Ex 6 | | | | | | |
| 19 | 24 | 2 | S | —OH | N-(biphenyl-3- yl)-6-(2-(2- hydroxyethylthio)- acetamido)- hexanamide | (MeOD-d4) δ (ppm) ¹H: 7.84-7.83 (m, 1H), 7.60-7.57 (m, 2H), 7.53-7.50 (m, 1H), 7.44-7.30 (m, 5H), 3.69 (t, J=6.5 Hz, 2H), 3.23 (t, J=6.9 Hz, 2H), 3.19 (s, 2H), 2.71 (t, J=6.2 Hz, 2H), 2.42 (t, J=7.2 Hz, 2H), 1.92-1.91 (m, 1H), 1.79-1.72 (m, 2H), 1.63-1.56 (m, 2H), 1.49-1.41 | |
| # (m, 2H). LRMS (ESI): (calc) 400.2; (found) 401.2 (MH)⁺. | 1 Step 1-5 Ex 1 Step 5 Ex 6 | | | | | | |
| 20 | 25 | 1 | S | 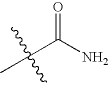 | 6-(2-(2-amino-2- oxoethylthio)- acetamido)-N- (biphenyl-3- yl)hexanamide | (DMSO-d6) δ (ppm) ¹H: 9.94 (s, 1H), 8.03 (t, J=7.1 Hz, 1H), 7.90 (s, 1H), 7.59-7.54 (m, 3H), 7.47-7.43 (m, 3H), 7.37-7.33 (m, 2H), 7.30-7.28 (m, 1H), 7.06 (br s, 1H), 3.19 (s, 2H), 3.17 (s, 2H), 3.05 (q, J=6.6 Hz, 2H), 2.32 (t, J=7.4 Hz, 2H), 1.64-1.59 (m, 2H), 1.48-1.41 (m, 2H), 1.35-1.29 | |
| # (m, 2H). LRMS (ESI): (calc) 413.2; (found) 414.2 (MH)⁺. | 1 Step 1-4 Ex 1 | | | | | | |
| 21 | 26 | 1 | S | 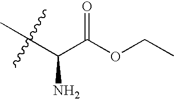 | (R)-ethyl 2- amino-3-(2-(6- (biphenyl-3- ylamino)-6- oxohexylamino)- 2-oxoethylthio)- propanoate | (DMSO-d6) δ (ppm) ¹H: 9.95 (s, 1H), 8.00 (t, J=.5 Hz, 1H), 7.90 (s, 1H), 7.59-7.56 (m, 3H), 7.45 (t, J=7.5 Hz, 2H), 7.37-7.33 (m, 2H), 7.30-7.28 (m, 1H), 4.07 (q, J=7.2 Hz, 2H), 3.10 (s, 2H), 3.05 (q, J=6.3 Hz, 2H), 2.80-2.67 (m, 2H), 2.32 (t, J=7.4 Hz, 2H), | |
| # 1.62-1.58 (m, 2H), 1.45-1.41 (m,2H), 1.34-1.30 (m, 2H), 1.19 (t, J=7.0 Hz, 3H). LRMS (ESI): (calc) 471.3; (found) 472.9 (MH)⁺. | 1 Step 1-5 Ex 1 | | | | | | |
| 22 | 27 | 1 | S | 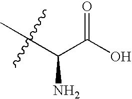 | (R)-2-amino-3- (2-(6-(biphenyl- 3-ylamino)-6- oxohexylamino)- 2-oxoethylthio)- propanoic acid | (DMSO-d6) δ (ppm) ¹H: 10.39 (s, 1H), 8.27 (t, J=6.7 Hz, 1H), 7.98 (s, 1H), 7.64-7.58 (m, 5H), 7.44 (1, J=7.6 Hz, 2H), 7.36-7.32 (m, 2H), 7.30-7.26 (m, 1H), 3.41-3.38 (m, 2H), 3.16 (dd, J=14.5, 10.0 Hz, 2H), 3.09-3.05 (m, 2H), 2.88-2.82 (m, 2H), 2.33 (t, J=7.0 Hz, | |
| # 2H), 1.63-1.56 (m, 2H), 1.48-1.42 (m, 2H), 1.36-1.31 (m, 2H). LRMS (ESI): (calc) 443.2; (found) 444.2 (MH)⁺. | 1 Step 1-6 Ex 1 | | | | | | |
| 23 | 28 | 2 | S | —NH₂ | 6-(2-(2- aminoethylthio)- acetamido)-N- (biphenyl-3- yl)hexanamide | (DMSO-d6) δ (ppm) ¹H: 9.95 (s, 1H), 8.02 (s, 1H), 7.90 (s, 1H), 7.59-7.53 (m, 3H), 7.45 (t, J=7.3 Hz, 2H), 7.35 (t, J=6.5 Hz, 2H), 7.30-7.28 (m, 1H), 3.10-3.01 (m, 4H), 2.74 (t, J=6.8 Hz, 2H), 2.32 (t, J=6.9 Hz, 2H), 1.91 (s, 2H), 1.79 (s, 2H), 1.62-1.58 (m, 2H), 1.46-1.42 (m, 2H), 1.36-1.29 | |
| # (m, 2H). LRMS (ESI): (calc) 399.2; (found) 400.7 (MH)⁺. | | | | | | | |
| 24 | 29 | — | S | 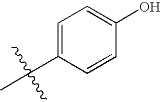 | N-(biphenyl-3- yl)-6-(2-(4- hydroxyphenyl- thio)-acetamido)- hexanamide | (DMSO-d6) δ (ppm) ¹H: 9.94 (s, 1H), 9.56 (s, 1H), 7.94-7.90 (m, 2H), 7.59-7.54 (m, 3H), 7.45 (t, J=7.6 Hz, 2H), 7.37-7.33 (m, 2H), 7.30-7.28 (m, 1H), 7.20 (d, J=8.8 Hz, 2H), 6.69 (d, J=8.7 Hz, 2H), 3.41 (s, 2H), 3.02 (q, J=6.5 Hz, 2H), 2.31 (t, J=7.2 Hz, 2H), 1.62-1.54 (m, 2H), | |
| # 1.41-1.34 (m, 2H), 1.29-1.24 (m, 2H). LRMS (ESI): (calc) 448.2; (found) 449.4 (MH)⁺. | 1 Step 1-4 Ex 1 | | | | | | |
| 25 | 30 | 1 | SO | 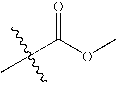 | methyl 2-(2-(6- (biphenyl-3- ylamino)-6- oxohexylamino)- 2- oxoethylsulfinyl)- acetate | (DMSO-d6) δ (ppm) ¹H: 9.96 (s, 1H), 8.28-8.29 (m, 1H), 7.90 (s, 1H), 7.59-7.54 (m, 3H), 7.45 (t, J=7.4 Hz, 2H), 7.36 (t, J=7.4 Hz, 2H), 7.30-7.28 (m, 1H), 4.08 (d, J=14.3 Hz, 1H), 3.91-3.80 (m, 2H), 3.69 (s, 3H), 3.68-3.66 (m, 2H), 3.10-3.08 (m, 2H), 2.32 (t, J=7.0 Hz, | |
| # 2H), 1.62-1.58 (m, 2H), 1.46-1.43 (m, 2H), 1.34-1.31 (m, 2H). LRMS (ESI): (calc) 444.2; (found) 445.5 (MH)⁺. | 1 Step 1-4 Ex 1 Step 6 Ex 2 | | | | | | |
| 26 | 31 | 2 | S | 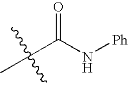 | N-(biphenyl-3- yl)-6-(2-(3-oxo-3- (phenylamino)- propylthio)- acetamido)- hexanamide | (DMSO-d6) δ (ppm) ¹H: 9.93 (s, 2H), 7.98 (t, J=5.5 Hz, 1H), 7.90 (s, 1H), 7.58-7.54 (m, 5H), 7.44 (t, J=7.2 Hz, 2H), 7.37-7.32 (m, 2H), 7.29-7.23 (m, 3H), 6.99 (td, J=7.3, 1.0 Hz, 1H), 3.11 (s, 2H), 3.06 (q, J=6.0 Hz, 2H), 2.82 (t, J=7.2 Hz, 2H), 2.61 (t, J=7.0 Hz, 2H), 2.31 | |
| # (t, J=7.2 Hz, 2H), 1.64-1.56 (m, 2H), 1.46-1.40 (m, 2H), 1.34-1.30 (m, 2H). MS: 503.3 (calc) 504.4 (found) LRMS (ESI): (calc) 503.3; (found) 504.4 (MH)⁺. | 1 Step 1-6 Ex 1 | | | | | | |
| 27 | 32 | 2 | SO | 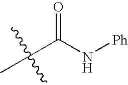 | N-(biphenyl-3- yl)-6-(2-(3-oxo-3- (phenylamino)- propylsulfinyl)- acetamido)- hexanamide | (MeOD-d4) δ (ppm) ¹H: 7.83 (s, 1H), 7.60-7.57 (m, 2H), 7.54-7.51 (m, 3H), 7.43-7.26 (m, 7H), 7.07 (1, J=7.4 Hz, 1H), 3.81-3.65 (m, 2H), 3.27 (t, J=6.7 Hz, 2H), 3.33-2.92 (m, 2H), 2.88 (t, J=6.8 Hz, 2H), 2.42 (t, J=7.4 Hz, 2H), 1.75 (quintet, J=7.8 Hz, 2H), 1.61 | |
| # (quintet, J=7.3 Hz, 2H), 1.50-1.54 (m, 2H). LRMS (ESI): (calc) 519.2; (found) 520.3 (MH)⁺. | 1 Step 1-6 Ex 1 Step 6 Ex 2 | | | | | | |
| 28 | 33 | — | S | 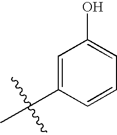 | N-(biphenyl-3- yl)-6-(2-(3- hydroxyphenyl- thio)-acetamido)- hexanamide | (MeOD-d4) δ (ppm) ¹H: 7.83 (s, 1H), 7.60-7.58 (m, 2H), 7.52 (dt, J=7.6, 1.9 Hz, 1H), 7.44-7.30 (m, 5H), 7.09 (t, J=7.7 Hz, 1H), 6.82-6.78 (m, 2H), 6.65-6.61 (m, 1H), 3.57 (s, 2H), 3.19 (t, J=6.8 Hz, 2H), 2.37 (t, J=7.4 Hz, 2H), 1.70-1.66 (m, 2H), 1.51-1.46 (m, 2H), 1.35-1.28 (m, | |
| # 2H). LRMS (ESI): (calc) 448.2 (found) 449.2 (MH)⁺. | 1 Step 1-4 Ex 1 | | | | | | |
| 29 | 34 | — | S | 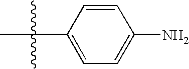 | 6-(2-(4- aminophenylthio)- acetamido)-N- (biphenyl-3- yl)hexanamide | (DMSO-d6) δ (ppm) ¹H: 9.94 (s, 1H), 7.90 (s, 1H), 7.87 (t, J=5.7 Hz, 1H), 7.59-7.54 (m, 3H), 7.45 (t, J=7.6 Hz, 2H), 7.37-7.33 (m, 2H), 7.30-7.26 (m, 1H), 7.07 (d, J=8.6 Hz, 2H), 6.47 (d, J=8.4 Hz, 2H), 5.24 (s, 2H), 3.31 (s, 2H), 3.01 (q, J=6.5 Hz, 2H), 2.31 (t, J=7.4 Hz, 2H), 1.60-1.54 | |
| # (m, 2H), 1.40-1.34 (m, 2H), 1.30-1.25 (m, 2H). LRMS (ESI): (calc) 447.2; (found) 448.3 (MH)⁺. | 1 Step 1-4 Ex 1 | | | | | | |
| 30 | 35 | — | S | 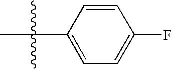 | N-(biphenyl-3- yl-6-(2-(4 fluorophenylthio)- acetamido)- hexanamide | (MeOD-d4) δ (ppm) ¹H: 7.84 (t, J=2.4 Hz, 1H), 7.60-7.57 (m, 2H), 7.52 (dt, J=7.5, 1.6 Hz, 1H), 7.44-7.30 (m, 7H), 7.07-7.02 (m, 2H), 3.52 (s, 2H), 3.17 (t, J=6.8 Hz, 2H), 2.38 (t, J=7.2 Hz, 2H), 1.72-1.67 (m, 2H), 1.50-1.44 (m, 2H), 1.36-1.30 (m, 2H). LRMS (ESI): (calc) 450.2; (found) | |
| # 451.3 (MH)⁺. LRMS (ESI): (calc); (found) (MH)⁺. | 1 Step 1-4 Ex 1 | | | | | | |
| 31 | 36 | — | S | 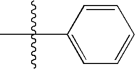 | N-(biphenyl-3- yl)-6-(2- (phenylthio)- acetamido)- hexanamide | (MeOD-d4) δ (ppm) ¹H 7.84 (s, 1H), 7.60-7.58 (m, 2H), 7.53-7.50 (m, 1H), 7.44-7.26 (m, 9H), 7.21-7.17 (m, 1H), 3.58 (s, 2H), 3.18 (t, J=6.9 Hz, 2H), 2.37 (t, J=7.2 Hz, 2H), 1.68 (quintet, J=7.8 Hz, 2H), 1.46 (quintet, J=7.8 Hz, 2H), 1.34-1.28 (m, 2H). LRMS (ESI): (calc) 432.2; (found) 433.3 | |
| # (MH)⁺. | 1 Step 1-4 Ex 1 | | | | | | |
| 32 | 37 | — | S | 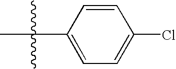 | N-(biphenyl-3- yl)-6-(2-(4- chlorophenylthio)- acetamido)- hexanamide | (MeOD-d4) δ (ppm) ¹H 7.84 (t, J=1.7 Hz, 1H), 7.60-7.57 (m, 2H), 7.52 (dt, J=7.6, 1.7 Hz, 1H), 7.44-7.27 (m, 9H), 3.58 (s, 2H), 3.18 (t, J=6.8 Hz, 2H), 2.38 (t, J=7.4 Hz, 2H), 1.69 (quintet, J=7.6 Hz, 2H), 1.48 (quintet, J=7.4 Hz, 2H), 1.36-1.30 (m, 2H). LRMS (ESI): (calc) 466.2; (found) | |
| # 467.2 (MH)⁺. | 1 Step 1-4 Ex 1 | | | | | | |
| 33 | 38 | — | S | 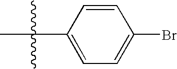 | N-(biphenyl-3- yl)-6-(2-(4- bromophenylthio)- acetamido)- hexanamide | (MeOD-d4) δ (ppm) ¹H 7.84 (s, 1H), 7.60-7.57 (m, 2H), 7.53-7.50 (m, 1H), 7.44-7.26 (m, 9H), 3.58 (s, 2H), 3.18 (t, J=6.7 Hz, 2H), 2.38 (t, J=7.4 Hz, 2H), 1.69 (quintet, J=7.6 Hz, 2H), 1.48 (quintet, J=7.3 Hz, 2H), 1.35-1.27 (m, 2H). LRMS (ESI): (calc) 511.1; 512.2 (found) (MH)⁺. | |
| # 1 Step 1-4 Ex 1 | | | | | | | |
| 34 | 39 | — | S | 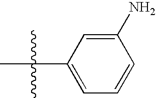 | 6-(2-(3- aminophenylthio)- acetamido)-N- (biphenyl-3- yl)hexanamide | (MeOD-d4) δ (ppm) ¹H 7.84 (s, 1H), 7.61-7.58 (m, 2H), 7.53-7.50 (m, 1H), 7.44-7.31 (m, 5H), 7.00 (t, J=7.8 Hz, 1H), 6.71 (t, J=1.8 Hz, 1H), 6.67-6.63 (m, 1H), 6.56-6.53 (m, 1H), 3.55 (s, 2H), 3.19 (t, J=6.8 Hz, 2H), 2.37 (t, J=7.4 Hz, 2H), 1.68 (quintet, J=7.3 Hz, 2H), 1.51-1.45 (m, 2H), | |
| # 1.34-1.28 (m, 2H). LRMS (ESI): (calc) 447.2; 447.7 (found) (MH)⁺. | 1 Step 1-4 Ex 1 | | | | | | |
| 35 | 40 | — | S | 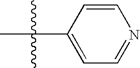 | N-(biphenyl-3- yl)-6-(2-(pyridin- 4- ylthio)acetamido)- hexanamide | (MeOD-d4) δ (ppm) ¹H: 8.34-8.30 (m, 2H), 7.87 (t, J=1.6 Hz, 1H), 7.64-7.58 (m, 2H), 7.57-7.53 (m, 1H), 7.47-7.30 (m, 7H), 3.80 (s, 2H), 3.25 (t, J=6.8 Hz, 2H), 2.40 (t, J=7.4 Hz, 2H), 1.78-1.68 (m, 2H), 1.62-1.52 (m, 2H), 1.44-1.35 (m, 2H). LRMS (ESI): (calc) 433.6; 434.4 (found) | |
| # (MH)⁺. | 1 Step 1-4 Ex 1 | | | | | | |
| 36 | 41 | — | S | 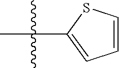 | N-(biphenyl-3- yl)-6-(2- (thiophen-2- ylthio)acetamido)- hexanamide | (MeOD-d4) δ (ppm) ¹H: 7.87 (t, J=1.8 Hz, 1H), 7.64-7.59 (m, 2H), 7.57-7.53 (m, 1H), 7.50-7.32 (m, 6H), 7.22-7.19 (m, 1H), 7.03-6.99 (m, 1H), 3.43 (s, 2H), 3.38 (s,[?]), 3.20 (t, J=6.8 Hz, 2H), 2.43 (t, J=7.2 Hz, 2H), 1.80-1.70 (m, 2H), 1.58-1.49 (m, 2H), 1.43-1.33 (m, 2H) LRMS | |
| # (ESI): (calc) 438.6; (found) 438.8 (MH)⁺. | 1 Step 1-4 Ex 1 | | | | | | |

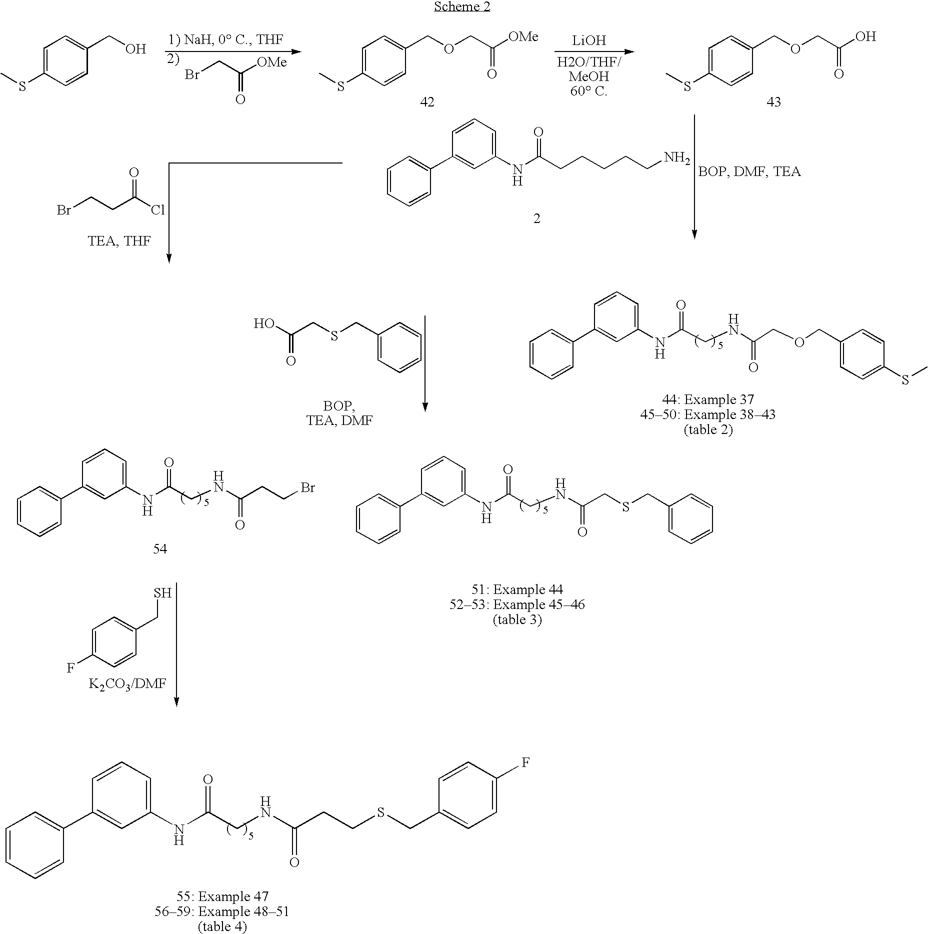

### EXAMPLE 37

### N-(biphenyl-3-yl)-6-(2-(4-(methylthio)benzyloxy)acetamido)hexanamide) (44)

### Step 1: methyl 2-(4-(methylthio)benzyloxy)acetate (42)

To a stirred solution of (4-(methylthio)phenyl)methanol (500 mg, 3.24 mmol) in THF (10 mL) at 0° C. was added sodium hydride (143 mg, 3.56 mmol). Bromomethyl acetate (3.24 mmol, 0.31 mL) was then added via a syringe and the reaction was allowed to warm to room temperature with stirring over 15 hours. The reaction mixture was quenched with water and extracted with ethyl acetate. The organic extract was dried (Na₂SO₄), filtered, and evaporated. The residue was purified by silica gel column chromatography with gradient of EtOAc (20-25%) in hexane to afford 42 (240 mg, 33%) as a clear oil.

¹H NMR: (CDCl₃) δ (ppm): 7.21 (d, J=8.6 Hz, 2H), 7.16 (d, J=8.4 Hz, 2H), 4.51 (s, 2H), 4.03 (s, 2H), 3.69 (s, 3H), 2.41 (s, 3H).

### Step 2: 2-(4-(methylthio)benzyloxy)acetic acid (43)

Following the same procedure as described for compound 5 (step 5, scheme 1, example 1) but substituting 42 for methyl ester 4, to afford 43 (160 mg, 60%) as a white solid. ¹H NMR: (CDCl₃) δ (ppm): 9.84 (br s, 1H), 7.45-7.32 (m, 4H), 4.73 (s, 2H), 4.27 (s, 2H), 2.63 (s, 3H).

### Step 3: N-(biphenyl-3-yl)-6-(2-(4-(methylthio)benzyloxy)acetamido)hexanamide) (44)

Following the same procedure as described for compound 1 (scheme 1, example 1) but substituting acid 43 for N-Boc-caproic acid, to afford 44 (160 mg, 15% from step 1-3) as a white solid. (DMSO-d6) δ (ppm) ¹H: 9.95 (s, 1H), 7.90 (t, J=1.8 Hz, 1H), 7.78 (t, J=5.8 Hz, 1H), 7.59-7.54 (m, 3H), 7.47-7.43 (m, 2H), 7.37-7.33 (m, 2H), 7.30-7.27 (m, 3H), 7.23-7.20 (m, 2H), 4.46 (s, 2H), 3.83 (s, 2H), 3.10 (q, J=7.0 Hz, 2H), 2.46 (s, 3H), 2.33 (t, J=7.5 Hz, 2H), 1.63-1.59 (m, 2H), 1.48-1.42 (m, 2H), 1.33-1.27 (m, 2H). LRMS (ESI): (calc) 476.3; (found) 477.3 (MH)⁺.

### EXAMPLE 38-43

Example 38-43 describe the preparation of compound 45-50 using the same procedures as described for compound 44 in Example 37, scheme 2. Characterization data are presented in a Table 2.

TABLE 2

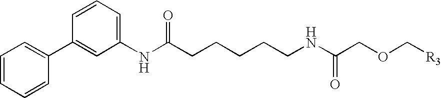

| Ex | Cpd | R₃ | Name | Characterization | Scheme |
|---|---|---|---|---|---|
| 38 | 45 | 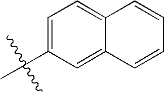 | N-(biphenyl-3-yl)-6- (2-(naphthalen-2- ylmethoxy)- acetamido)- hexanamide | (DMSO-d6) δ (ppm) ¹H: 9.96 (s, 1H), 7.92-7.84 (m, 6H), 7.60-7.55 (m, 3H), 7.52-7.49 (m, 3H), 7.48-7.43 (m, 2H), 7.38-7.34 (m, 2H), 7.31-7.28 (m, 1H), 4.69 (s, 2H), 3.93 (s, 2H), 3.13 (q, J=6.7 Hz, 2H), 2.34 (t, J=7.4 Hz, 2H), 1.64-1.60 (m, 2H), 1.50-1.46 (m, 2H), 1.33-1.29 (m, 2H). | |
| # LRMS (ESI): (calc) 480.3; (found) 481.3 (MH)⁺. | 2 Step 1-3 Ex 26 | | | | |
| 39 | 46 | 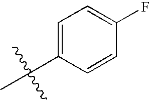 | N-(biphenyl-3-yl)-6- (2-(4- fluorobenzyloxy)- acetamido)- hexanamide | (DMSO-d6) δ (ppm) ¹H: 9.95 (s, 1H), 7.90 (s, 1H), 7.80 (t, J=5.5 Hz, 1H), 7.59-7.54 (m, 3H), 7.47-7.28 (m, 7H), 7.18-7.13 (m, 2H), 4.49 (s, 2H), 3.86 (s, 2H), 3.10 (q, J=6.7 Hz, 2H), 2.33 (t, J=7.4 Hz, 2H), 1.65-1.57 (m, 2H), 1.48-1.43 (m, 2H), 1.33-1.27 (m, 2H). LRMS (ESI): | |
| # (calc) 448.2; (found) 449.2 (MH)⁺. | 2 Step 1-3 Ex 26 | | | | |
| 40 | 47 | 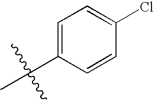 | N-(biphenyl-3-yl)-6- (2-(4- chlorobenzyloxy)- acetamido)- hexanamide | (DMSO-d6) δ (ppm) ¹H: 9.94 (s, 1H), 7.89 (s, 1H), 7.80 (t, J=5.9 Hz, 1H), 7.58-7.53 (m, 3H), 7.46-7.42 (m, 2H), 7.40-7.32 (m, 6H), 7.30-7.27 (m, 1H), 4.49 (s, 2H), 3.86 (s, 2H), 3.09 (q, J=6.8 Hz, 2H), 2.32 (t, J=7.0 Hz, 2H), 1.64-1.56 (m, 2H), 1.47-1.42 (m, 2H), 1.33-1.25 (m, 2H). LRMS (ESI): | |
| # (calc) 464.2; (found) 465.3 (MH)⁺. | 2 Step 1-3 Ex 26 | | | | |
| 41 | 48 | 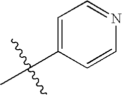 | N-(biphenyl-3-yl)-6- (2-(pyridin-4- ylmethoxy)- acetamido)- hexanamide | (CDCl₃-d6) δ (ppm) ¹H: 8.57 (d, J=5.9 Hz, 2H), 7.81 (s, 1H), 7.72 (s, 1H), 7.58-7.50 (m, 3H), 7.42-7.30 (m, 5H), 7.24 (d, J=6.1 Hz, 2H), 6.61 (br s, 1H), 4.56 (s, 2H), 3.99 (s, 2H), 3.33 (q, J=6.8 Hz, 2H), 2.40 (t, J=7.2 Hz, 2H), 1.82-1.74 (m, 2H), 1.61-1.55 (m, 2H), 1.46-1.40 (m, 2H) LRMS | |
| # (ESI): (calc) 431.3; (found) 432.3 (MH)⁺. | 2 Step 1-3 Ex 26 | | | | |
| 42 | 49 | 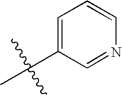 | N-(biphenyl-3-yl)-6- (2-(pyridin-3- ylmethoxy)- acetamido)- hexanamide | (DMSO-d6) δ (ppm) ¹H: 9.95 (s, 1H), 8.56 (s, 1H), 8.48 (d, J=3.1 Hz, 1H), 7.90 (s, 1H), 7.82 (t, J=5.6 Hz, 1H), 7.77 (d, J=7.8 Hz, 1H), 7.58-7.53 (m, 3H), 7.45 (t, J=7.3 Hz, 2H), 7.38-7.33 (m, 3H), 7.29-7.27 (m, 1H), 4.54 (s, 2H), 3.90 (s, 2H), 3.10 (q, J=6.5 Hz, 2H), 2.32 (t, J=7.2 Hz, 2H), | |
| # 1.61-1.59 (m, 2H), 1.47-1.42 (m, 2H), 1.33-1.27 (m, 2H) LRMS (ESI): (calc) 431.3; (found) 432.3 (MH)⁺. | 2 Step 1-3 Ex 26 | | | | |
| 43 | 50 | 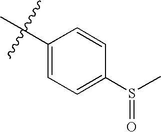 | N-(biphenyl-3-yl)-6- (2-(4- (methylsulfinyl)- benzyloxy)- acetamido)- hexanamide | (DMSO-d6) δ (ppm) ¹H: 10.03 (s, 1H), 7.95 (s, 1H), 7.89 (t, J=5.6 Hz, 1H), 7.68 (d, J=8.0 Hz, 2H), 7.56-7.63 (m, 5H), 7.48 (t, J=7.6 Hz, 2H), 7.39 (t, J=7.5 Hz, 2H), 7.32 (d, 7.8 Hz, 1H), 4.62 (s, 2H), 3.94 (s, 2H), 3.14 (q, J=12.9, 6.5 Hz, 2H), 2.76 (s, 3H), 2.37 (t, J=7.3 Hz, 2H), 1.60-1.70 | |
| # (m, 2H), 1.45-1.55 (m, 2H), 1.28-1.39 (m, 2H). LRMS (ESI): (calc) 492.6; (found) 493.0 (MH)⁺. | 2 Step 1-3 Ex 26 1 Step 6 Ex 2 | | | | |

### EXAMPLE 44

### 6-(2-(benzylthio)acetamido)-N-(biphenyl-3-yl)hexanamide (51)

### Step 1: 6-(2-(benzylthio)acetamido)-N-(biphenyl-3-yl)hexanamide (51)

Following the same procedure as described for compound 1 (scheme 1, example 1) but substituting 2-(benzylthio)acetic acid for N-Boc-caproic acid to afford 51 (142 mg, 64%) as a white solid. (DMSO-d6) δ (ppm) ¹H, 10.00 (br s,1H), 7.99 (t, J=5.5 Hz, 1H), 7.94 (br t, 1H), 7.60 (m,3H), 7.49 (t, J=7.8 Hz, 2H), 7.39 (t, J=7.8 Hz, 2H), 7.36-7.30 (m,4H), 7.28-7.22 (m,1H), 3.81 (s,2H), 3.09 (q, J=12.5, 6.7 Hz, 2H), 3.03 (s,2H), 2.37 (t, J=7.4 Hz, 2H), 1.70-1.60 (m,2H), 1.52-1.40 (m,2H), 1.40-1.30 (m,2H). LRMS (ESI): (calc) 446.6; (found) 447.2 (MH)⁺.

### EXAMPLE 45-46

Example 45-46 describe the preparation of compound 52-53 using the same procedures as described for compound 51 in Example 44. Characterization data are presented in a table 3.

TABLE 3

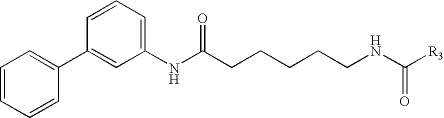

| Ex | Cpd | R₃ | Name | Characterization | Scheme |
|---|---|---|---|---|---|
| 45 | 52 | 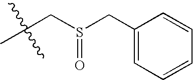 | 6-(2- (benzylsulfinyl)- acetamido)-N- (biphenyl-3- yl)hexanamide | (DMSO-d6) ≃ (ppm) ¹H: 10.01 (s, 1H), 8.30 (t, J=5.5 Hz, 1H), 7.95 (s, 1H), 7.64-7.57 (m, 3H), 7.49 (t, J=7.8 Hz, 2H), 7.42-7.36 (m, 4H), 7.35-7.31 (m, 3H), 4.26 (d, J=12.7 Hz, 1H), 4.02 (d, J=12.9 Hz, 1H), 3.68 (d, J=13.1 Hz, 1H), 3.46 (d, J=13.1 Hz, 1H), 3.14 (m, 2H), 2.36 (t, J=7.3 Hz, 2H), | |
| # 1.70-1.60 (m, 2H), 1.54-1.43 (m, 2H), 1.40-1.30 (m, 2H) LRMS (ESI): (calc) 462.6; (found) 463.2 (MH)⁺. | 2 Step 1 Ex 33 1 Step 6 Ex 2 | | | | |
| 46 | 53 | 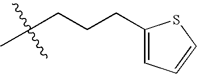 | N-(biphenyl-3-yl)- 6-(4-(thiophen-2- yl)butanamido)- hexanamide | (DMSO-d6) δ (ppm) 1H: 9.94 (s, 1H), 7.90 (t, J=1.8 Hz, 1H), 7.78 (t, J=5.7 Hz, 1H), 7.59-7.54 (m, 3H), 7.47-7.43 (m, 2H), 7.37-7.30 (m, 2H), 7.30-7.27 (m, 2H), 6.90 (dd, J=5.1, 3.3 Hz, 1H), 6.81-6.80 (m, 1H), 3.04 (q, J=6.7 Hz, 2H), 2.75 (t, J=7.4 Hz, 2H), 2.32 (t, J=7.2 Hz, 2H), 2.10 (t, J=7.4 Hz, | |
| # 2H), 1.85-1.77 (m, 2H), 1.63-1.57 (m, 2H), 1.45-1.39 (m, 2H), 1.34-1.28 (m, 2H) LRMS (ESI): (calc) 434.3; (found) 435.3 (MH)⁺. | 2 Step 1 Ex 33 | | | | |

### EXAMPLE 47

### N-(biphenyl-3-yl)-6-(3-(4-fluorobenzylthio)propanamido)hexanamide (55)

### Step 1: N-(biphenyl-3-yl)-6-(3-bromopropanamido)hexanamide (54)

To a solution of 2 (2.04 g, 7.22 mmol) in THF (20 mL) was added 3-bromo-propionyl chloride (0.73 mL, 7.22 mmol) and triethylamine (3.02 mL, 21.7 mmol). After stirring at room temperature for 30 min, the solution was diluted with brine, extracted with EtOAc.

The organic extract was dried (Na₂SO₄), filtered, and evaporated. The residue was purified by silica gel column chromatography with gradient of EtOAc (25-100%) in Hexane to afford 54 (270 mg, 9%) as a white solid. LRMS (ESI): (calc.) 417.5; (found) 418.2 (MH)⁺.

### Step 2: N-(biphenyl-3-yl)-6-(3-(4-fluorobenzylthio)propanamido)hexanamide (55)

To a solution of 54 (132 mg, 0.32 mmol) in DMF (5 mL) was added (4-fluorophenyl)-methanethiol (0.04 mL, 0.32 mmol) and K₂CO₃ (131 mg, 0.95 mmol). The resulting solution was stirred for 16 h at 60° C. prior to removal of the solvent. The residue was purified by silica gel column chromatography with gradient of EtOAc (25-100%) in Hexane to afford 55 (109 mg, 71%) as a light brown solid. (DMSO-d6) δ (ppm) ¹H: 9.99 (s,1H), 7.96 (s,1H), 7.91 (m,1H), 7.65-7.57 (m,3H), 7.48 (t, J=7.8 Hz, 2H), 7.42-7.30 (m,5H), 7.14 (t, J=8.6 Hz, 2H), 3.74 (s,2H), 3.09 (m,2H), 2.59 (t, J=7.0 Hz, 2H), 2.37 (t, J=7.0 Hz, 4H), 1.70-1.59 (m,2H), 1.53-1.42 (m,2H), 1.40-1.30 (m,2H). LRMS (ESI): (calc.) 478.6; (found) 479.6 (MH)⁺.

### EXAMPLE 48-51

Example 48-51 describe the preparation of compound 54-59 using the same procedures as described for compound 55 in Example 47. Characterization data are presented in a Table 4.

TABLE 4

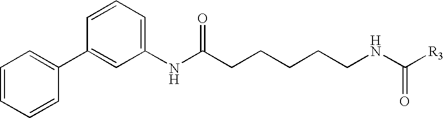

| Ex | Cpd | R₃ | Name | Characterization | Scheme |
|---|---|---|---|---|---|
| 48 | 56 | 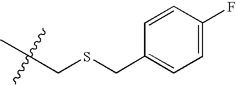 | N-(biphenyl-3-yl)-6- (2-(4-fluorobenzyl- thio)acetamido)- hexanamide | (DMSO-d6) δ (ppm) ¹H: 10.06 (s, 1H), 8.08 (m, 1H), 7.96 (s, 1H), 7.64-7.58 (m, 3H), 7.49 (t, J=7.6 Hz, 2H), 7.42-7.30 (m, 5H), 7.22 (t, J=8.8 Hz, 2H), 4.17 (d, J=12.7 Hz, 1H), 3.97 (d, J=12.9 Hz, 1H), 3.08 (q, J=12.5, 6.4 Hz, 2H), 3.04-2.94 (m, 1H), 2.80-2.70 (m, 1H), 2.52 (m, 2H), 2.36 (t, | |
| # J=7.2 Hz, 2H), 1.70-1.58 (m, 2H), 1.52-1.41 (m, 2H), 1.40-1.29 (m, 2H) LRMS (ESI): (calc.) 494.6; (found) 495.3 (MH)⁺. | 2 Step 1-2 Ex 36 | | | | |
| 49 | 57 | 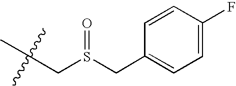 | N-(biphenyl-3-yl)-6- (2-(4-fluorobenzyl- sulfinyl)-acetamido)- hexanamide | (DMSO-d6) δ (ppm) ¹H: 10.12 (s, 1H), 8.35 (br t, 1H), 7.96 (s, 1H), 7.65-7.59 (m, 3H), 7.49 (t, J=7.6 Hz, 2H), 7.42-7.30 (m, 5H), 7.23 (t, J=8.8 Hz, 2H), 4.26 (d, J=13.1 Hz, 1H), 4.02 (d, J=12.9 Hz, 1H), 3.68 (d, J=13.1 Hz, 1H), 3.43 (d, J=13.3 Hz, 1H), 3.16-3.08 (m, 2H), 2.37 (t, J=7.2 Hz, | |
| # 2H),1.69-1.57 (m, 2H), 1.52-1.42 (m, 2H), 1.40-1.30 (m, 2H) LRMS (ESI): (calc.) 480.6; (found) 481.2 (MH)⁺. | 2 Step 1-2 Ex 36 1 Step 6 Ex 2 | | | | |
| 50 | 58 | 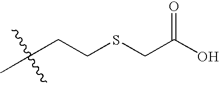 | 2-(3-(6-(biphenyl-3- ylamino)-6- oxohexylamino)-3- oxopropylthio)- acetic acid | (DMSO-d6) δ (ppm) ¹H: 9.95 (s, 1H), 7.90 (s, 1H), 7.87 (t, J=5.5 Hz, 1H), 7.60-7.53 (m, 3H), 7.45 (t, J=7.8 Hz, 2H), 7.37-7.32 (m, 2H), 7.28 (d, J=7.6 Hz, 1H), 3.23 (s, 2H), 3.03 (q, J=12.5, 6.7 Hz, 2H), 2.74 (t, J=7.0 Hz, 2H), 2.32 (m, 4H), 1.65-1.54 (m, 2H), 1.48-1.37 (m, 2H), 1.36-1.24 | |
| # (m, 2H) LRMS (ESI): (calc.) 428.5; (found) 429.5 (MH)⁺. | 2 Step 1-2 Ex 36 1 Step 5 Ex 1 | | | | |
| 51 | 59 | 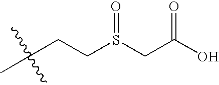 | 2-(3-(6-(biphenyl-3- ylamino)-6- oxohexylamino)-3- oxopropyl- sulfinyl)acetic acid | (DMSO-d6) δ (ppm) ¹H: 10.00 (s, 1H), 8.05 (m, 1H), 7.94 (s, 1H), 7.65-7.57 (m, 3H), 7.49 (t, J=7.4 Hz, 2H), 7.43-7.36 (m, 2H), 7.32 (d, J=7.8 Hz, 1H), 3.91 (d, J=14.3 Hz, 1H), 3.64 (d, J=14.3 Hz, 1H), 3.15-3.04 (m, 4H), 3.00-2.92 (m, 2H), 2.36 (t, J=7.2 Hz, 2H), 1.69-1.58 (m, 2H), | |
| # 1.52-1.42 (m, 2H), 1.40-1.30 (m, 2H) LRMS (ESI): (calc.) 444.5; (found) 445.4 (MH)⁺. | 2 Step 1-2 Ex 36 1 Step 5 Ex 1 1 Step 6 Ex 2 | | | | |

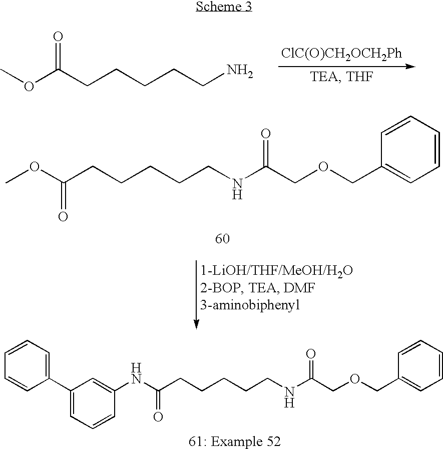

### EXAMPLE 52

### 6-(2-(benzyloxy)acetamido)-N-(biphenyl-3-yl)hexanamide (61)

### Step 1: methyl 6-(2-(benzyloxy)acetamido)hexanoate (60)

Following the same procedure as described for compound 54 (scheme 2, example 47) but substituting amine 5-Aminohexanoic for acid 2 and to afford 60 (1.60 g, 99%) as a yellow oil. LRMS (ESI): (calc.) 293.4; (found) 294.1 (MH)⁺.

### Step 2-3: 6-(2-(benzyloxy)acetamido)-N-(biphenyl-3-yl)hexanamide (61)

Following the same procedure as described for compound 5 (step 5, scheme 1, example 1) but substituting ester 60 for 4 to afford 6-(2-(benzyloxy)acetamido)hexanoic acid (1.2 g, 62%) as an off white solid.

Following the same procedure as described for compound 1 (step 1, scheme 1, example 1) but substituting acid 60 for N-Boc-caproic acid to afford 61 (1.14 g, 62%) as a white solid. (DMSO-d6) δ (ppm) ¹H: 9.96 (s, 1H), 7.93 (s, 1H), 7.79 (s, 1H), 7.60-7.56 (m, 3H), 7.47 (t, J=7.5 Hz, 2H), 7.35-7.30 (m, 7H), 4.52 (s, 2H), 3.87 (s, 2H), 3.11-3.10 (m, 2H), 2.33 (t, J=7.5 Hz, 2H), 1.61 (t, J=6.5 Hz, 2H), 1.47 (t, J=7.0 Hz, 2H), 1.32-1.29 (m, 2H). LRMS (ESI): (calc.) 430.5; (found) 431.3 (MH)⁺.

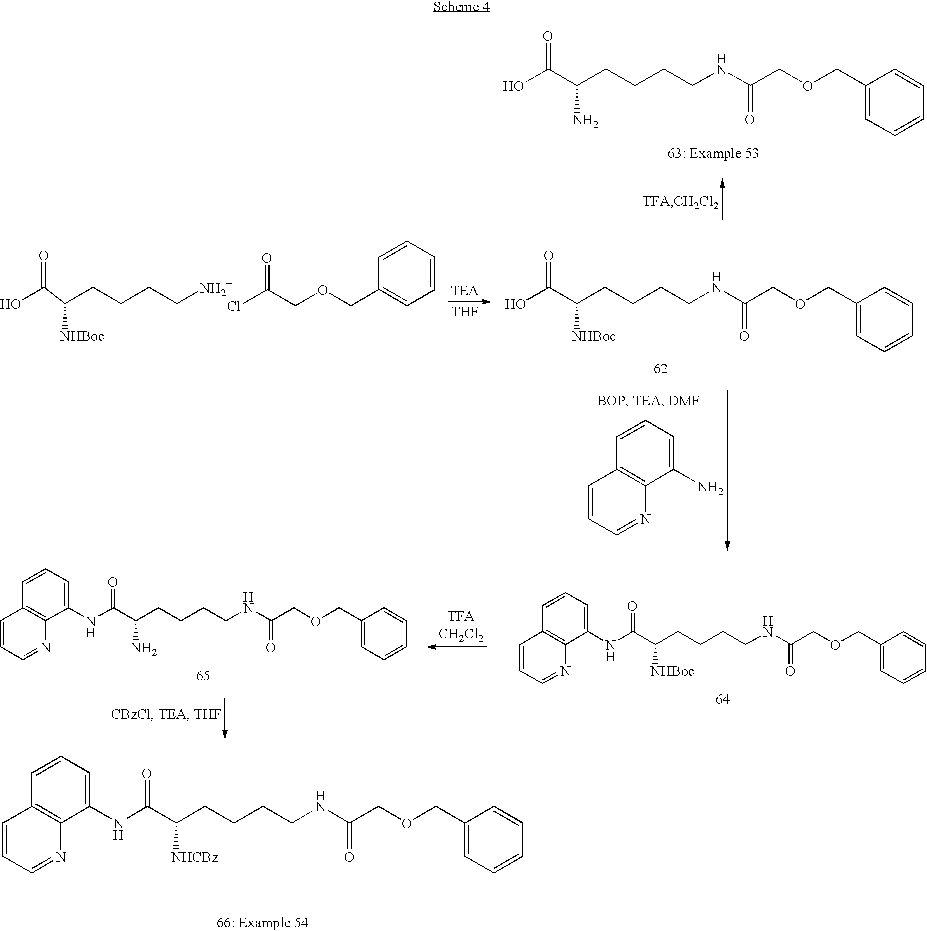

### EXAMPLE 53

### (S)-2-amino-6-(2-(benzyloxy)acetamido)hexanoic acid (63)

### Step 1: (S)-6-(2-(benzyloxy)acetamido)-2-(tert-butoxycarbonylamino)hexanoic acid (62)

Following the same procedure as described for compound 54 (scheme 2, example 47) but substituting (S)-6-Amino-2-(tert-butoxycarbonylamino)hexanoic acid for amine 2 and using 2 eq of 2-(benzyloxy)acetyl chloride to afford 62 (311 mg, 66%) as a white foam. LRMS (ESI): (calc.) 394.5; (found) 395.2 (MH)⁺.

### Step 2: (S)-2-amino-6-(2-(benzyloxy)acetamido)hexanoic acid (63)

Following the same procedure as described for compound 9 (step 5, scheme 1, example 4) but substituting 62 for 7a to afford 63 (35 mg, 29%) as a white solid. (DMSO-d6) δ (ppm) ¹H: 7.85 (m,1H), 7.40 (m,3H), 7.37-7.30 (m,2H), 4.56 (s,2H), 3.90 (s,2H), 3.14-3.08 (m,4H), 1.80-1.70 (m,1H), 1.66-1.55 (m, 1H), 1.48-1.38 (m,2H), 1.38-1.26 (m,2H).

LRMS (ESI): (calc.) 294.3; (found) 295.2 (MH)⁺.

### EXAMPLE 54

### (S)-benzyl 6-(2-(benzyloxy)acetamido)-1-oxo-1-(quinolin-8-ylamino)hexan-2-ylcarbamate (66)

### Step 2: (S)-tert-butyl 6-(2-(benzyloxy)acetamido)-1-oxo-1-(quinolin-8-ylamino)hexan-2-ylcarbamate (64)

Following the same procedure as described for compound 1 (step 1, scheme 1, example 1) but substituting 62 for N-Boc-caproic acid to afford 64 (127 mg, 69%) as a white solid. (DMSO-d6) δ (ppm) ¹H: 10.51 (br s,1H), 8.90 (dd, J=4.1, 1.4 Hz, 1H), 8.66 (d, J=7.6 Hz, 1H), 8.43 (d, J=8.2 Hz, 1H), 7.86 (t, J=5.1 Hz, 1H), 7.74-7.64 (m,3H), 7.61 (t, J=8.0 Hz, 1H), 7.39 (m,4H), 7.36-7.28 (m,1H), 4.56 (s,2H), 4.10 (m,1H), 3.89 (s,2H), 3.14 (m,2H), 1.94-1.84 (m,1H), 1.76-1.68 (m,1H), 1.50-1.40 (br s,11H), 1.32-1.26 (m,2H). LRMS (ESI): (calc.) 520.6; (found) 521.2 (MH)⁺.

### Step 3: (S)-2-amino-6-(2-(benzyloxy)acetamido)-N-(quinolin-8-yl)hexanamide (65)

Following the same procedure as described for compound 9 (step 5, scheme 1, example 4) but substituting 64 for 7a to afford 65 (75 mg, 92%) as a light green solid. (DMSO-d6) δ (ppm) ¹H: 11.30 (br s,1H), 8.96 (d, J=4.1 Hz, 1H), 8.67 (d, J=7.6 Hz, 1H), 8.43 (d, J=8.2 Hz, 1H), 7.84 (t, J=5.4 Hz, 1H), 7.72 (d, J=8.61 Hz, 1H), 7.66 (m,1H), 7.61 (t, J=7.6 Hz, 1H), 7.35 (m,4H), 7.33-7.28 (m,1H), 4.51 (s,2H), 3.86 (s,2H), 3.13 (m,2H), 1.94-1.84 (m,1H), 1.82-1.72 (m,1H), 1.56-1.40 (m,4H), 1.30-1.26 (m,1H). LRMS (ESI): (calc.) 420.5; (found) 421.3 (MH)⁺.

### Step 4: (S)-benzyl 6-(2-(benzyloxy)acetamido)-1-oxo-1-(quinolin-8-ylamino)hexan-2-ylcarbamate (66)

To a solution of 65 (81 mg, 0.192 mmol) in THF (5 mL) was added benzyl chloroformate (0.03 mL, 0.192 mmol) and triethylamine (0.15 mL, 1.08 mmol). The resulting solution was stirred at room temperature for 5 min prior to dilution with brine. The aqueous layer was then extracted with EtOAc. The organic extract was dried (Na₂SO₄), filtered, and evaporated. The residue was purified by silica gel column chromatography with gradient of EtOAc (25-100%) in Hexane to afford 66 (44 mg, 42%) as a white solid. (DMSO-d6) δ (ppm) ¹H: 10.50 (br s,1H), 8.87 (d, J=2.9 Hz, 1H), 8.66 (d, J=7.4 Hz, 1H), 8.44 (d, J=8.0 Hz, 1H), 8.15 (d, J=6.8 Hz, 1H), 7.86 (m,1H), 7.73-7.64 (m,2H), 7.61 (t, J=8.0 Hz, 1H), 7.38 (m,5H), 7.32 (m,3H), 5.17 (d, J=12.7 Hz, 1H), 5.07 (d, J=12.5 Hz, 1H), 4.55 (s,2H), 4.27 (m,1H), 3.90 (s,2H), 3.14 (m,2H), 1.98-1.83 (m,1H), 1.80-1.70 (m,1H), 1.40-1.38 (m,4H). LRMS (ESI): (calc.) 554.6; (found) 555.9 (MH)⁺.

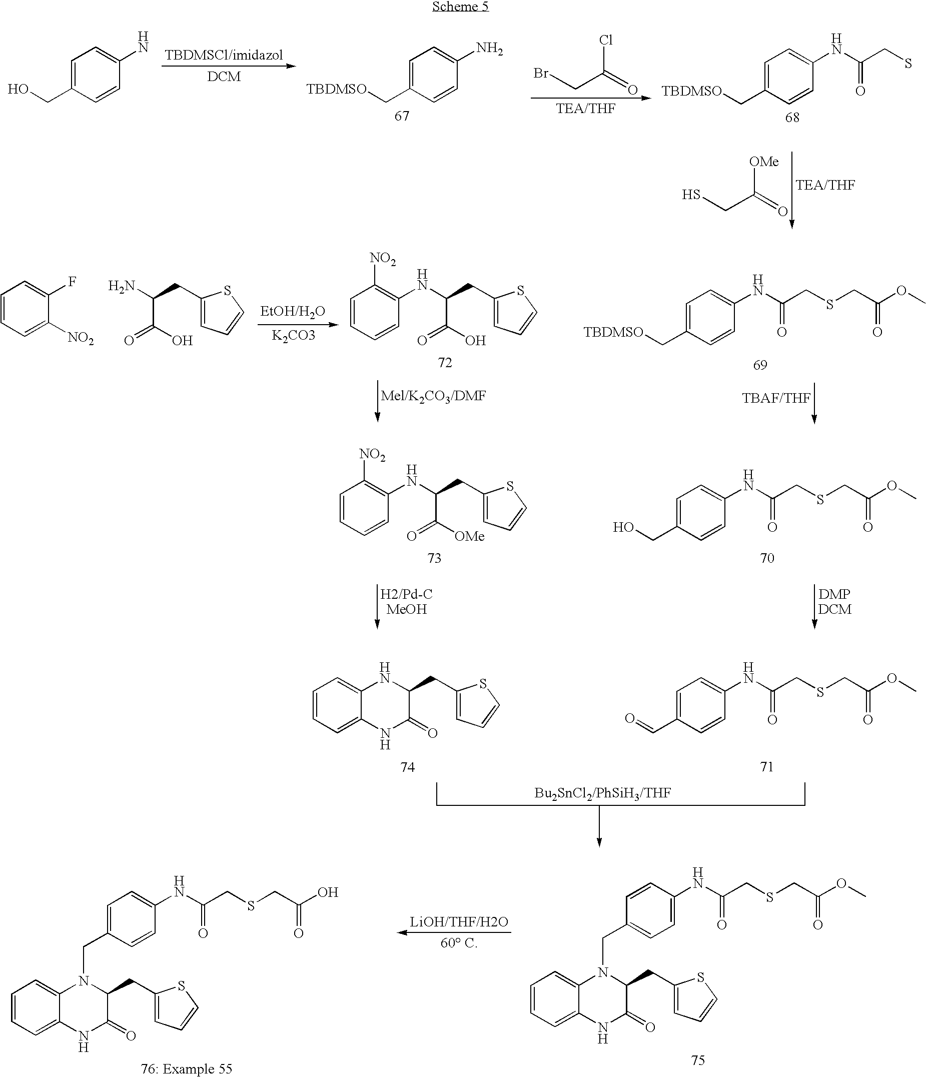

### EXAMPLE 55

### (S)-2-(2-oxo-2-(4-((3-oxo-2-(thiophen-2-ylmethyl)-3,4-dihydroquinoxalin-H (2H)-yl)methyl)phenylamino)ethylthio)acetic acid (76)

### Step 1: 4-((tert-butyldimethylsilyloxy)methyl)benzenamine (67)

To a solution of (4-Aminophenyl)methanol (1.50 g, 12.18 mmol) in CH₂Cl₂ (20 mL) was added TBDMSCl (1.84 g, 12.18 mmol) and imidazole (0.91 g, 13.40 mmol). After stirring for 1 h at room temperature, the solution was diluted with brine, and extracted with EtOAc. The organic extract was dried (Na₂SO₄), filtered, and evaporated. The residue was purified by silica gel column chromatography with gradient of EtOAc (25-100%) in Hexane to afford 67 (2.76 g, 96%) as a light yellow oil. LRMS (ESI): (calc.) 237.4; (found) 238.2 (MH)⁺.

### Step 2: 2-bromo-N-(4-((tert-butyldimethylsilyloxy)methyl)phenyl)acetamide (68)

Following the same procedure as described for compound 3b (step 3, scheme 1, example 1) but substituting amine 67 for amine 2 to afford 68 (1.72 g, 41%) as a white solid. LRMS (ESI): (calc.) 358.4; (found) 359.2 (MH)⁺.

### Step 3: methyl 2-(2-(4-((tert-butyidimethylsilyloxy)methyl)phenylamino)-2-oxoethylthio)acetate (69)

Following the same procedure as described for compound 4 (step 4, scheme 1, example 1) but substituting compound 68 for compound 3b to afford 69 (960 mg, 99%) as a light yellow oil. LRMS (ESI): (calc.) 383.5; (found) 384.2 (MH)⁺.

### Step 4: methyl 2-(2-(4-(hydroxymethyl)phenylamino)-2-oxoethylthio)acetate (70)

To a solution of 69 (0.963 g, 2.51 mmol) in THF (20 mL) was added TBAF (2.76 mL, 2.76 mmol). The resulting solution was stirred at room temperature for 2 h prior to dilution with brine, and extraction with EtOAc. The organic extracts were dried (Na₂SO₄), filtered, and evaporated. The residue was purified by silica gel column chromatography with gradient of EtOAc (25-100%) in Hexane to afford 70 (170 mg, 25%) as a light yellow oil. (DMSO-d6) δ (ppm) ¹H, 10.08 (s,1H), 7.53 (d, J=8.4 Hz, 2H), 7.26 (d, J=8.4 Hz, 2H), 5.14 (t, J=3.9 Hz, 1H), 4.46 (d, J=5.7 Hz, 2H), 3.66 (s,3H), 3.56 (s,2H), 3.45 (s,2H). LRMS (ESI): (calc.) 269.3; (found) 270.1 (MH)⁺.

### Step 5: methyl 2-(2-(4-formylphenylamino)-2-oxoethylthio)acetate (71)

To a solution of 70 (170 mg, 0.63 mmol) in CH₂Cl₂ (3 mL) was added Dess-Martin periodinane (290 mg, 0.69 mmol). The resulting solution was stirred at room temperature for 30 min prior to dilution with brine, and extraction with EtOAc. The organic extracts were dried (Na₂SO₄), filtered, and evaporated. The residue was purified by silica gel column chromatography with gradient of EtOAc (25-100%) in Hexane to afford 71 (112 mg, 67%) as a light yellow oil. LRMS (ESI): (calc.) 267.3; (found) 268.2 (MH)⁺.

### Synthesis of (S)-3-(thiophen-2-ylmethyl)-3,4-dihydroquinoxalin-2(1H)-one (74). Step 1-3

### Step 1: (S)-2-(2-nitrophenylamino)-3-(thiophen-2-yl)propanoic acid (72)

To a solution of (L)-2-amino-3-thiophen-2-yl-propionic acid (965 mg, 5.64 mmol) and 1-fluoro-2-nitrobenzene (0.59 mL, 5.64 mmol) in EtOH/H₂O (5:1, 12 mL) at room temperature was added potassium carbonate (1.56 g, 11.28 mmol). The resulting solution was heated at 100° C. for 16 h. After cooling, the solution was filtered, and evaporated to give 72 in quantitative yield. The crude was used in the subsequent reaction without further purification. LRMS (ESI): (calc.) 292.3; (found) 293.1 (MH)⁺.

### Step 2: (S)-methyl 2-(2-nitrophenylamino)-3-(thiophen-2-yl)propanoate (73)

To a solution of 72 (1.65 g, 5.64 mmol) in DMF (10 mL) was added potassium carbonate (3.12 g, 22.56 mmol) and methyl iodide (1.06 mL, 16.92 mmol). The resulting solution stirred at room temperature for 16 h. Following extraction from brine with EtOAc, the organic extracts were evaporated to give residue 73 in near quantitative yield. This material was used in the subsequent reaction without further purification. LRMS (ESI): (calc.) 306.3; (found) 307.2 (MH)⁺.

### Step 3: ((S)-3-(thiophen-2-ylmethyl)-3,4-dihydroquinoxalin-2(1H)-one (74)

To a solution of 73 (1.73 g, 5.64 mmol) in MeOH/EtOAc (2:1, 15 mL) was added 10% Pd/C (620 mg, 0.564 mmol). The resulting mixture was stirred under hydrogen atmosphere for 16 h, filtered through a pad of celite, and concentrated. The residue was purified by trituration with EtOAc:Hexane to afford 74 (1.31 g, 95%) as a light orange crystalline solid. LRMS (ESI): (calc.) 244.3; (found) 245.1 (MH)⁺.

### Step 6: (S)-methyl 2-(2-oxo-2-(4-((3-oxo-2-(thiophen-2-ylmethyl)-3,4-dihydroquinoxalin-1(2H)-yl)methyl)phenylamino)ethylthio)acetate (75)

To a solution of 74 (102 mg, 0.419 mmol) in THF (1.5 mL) was added aldehyde 710 (112 mg, 0.419 mmol), dibutyltin dichloride (13 mg, 0.0419 mmol) and triphenyl silane (0.06 mL, 0.461 mmol). The resulting solution was stirred at room temperature for 16 h and then evaporated The residue was purified by silica gel column chromatography with gradient of EtOAc (25-100%) in Hexane to afford 75 (113 mg, 54%) as a white foam. ¹H (DMSO-d6) δ (ppm) ¹H, 10.48 (s,1H), 10.10 (s,1H), 7.50 (d, J=8.2 Hz, 2H), 7.33 (d, J=5.1 Hz, 1H), 7.17 (d, J=8.4 Hz, 2H), 6.91 (m,1H), 6.75-6.86 (m,3H), 6.64-6.74 (m,2H), 4.54 (d, J=15.1 Hz, 1H), 4.14 (d, J=15.1 Hz, 1H), 4.07 (t, J=6.1 Hz, 1H), 3.64 (s,3H), 3.54 (s,2H), 3.43 (s,2H), 2.98-3.10 (m,2H). MS: (calc.) 495.6; (obt.) 496.2 (MH)⁺.

LRMS (ESI): (calc.) 495.6; (found) 496.2 (MH)⁺.

### Step 7: (S)-2-(2-oxo-2-(4-((3-oxo-2-(thiophen-2-ylmethyl)-3,4-dihydroquinoxalin-1(2H)-yl)methyl)phenylamino)ethylthio)acetic acid (76)

Following the same procedure as described for compound 5 (step 5, scheme 1, example 1) but substituting compound 75 for compound 4 to afford 76 (34 mg, 35%) as a light orange foam. (DMSO-d6) δ (ppm) ¹H: 12.65 (br s,1H), 10.48 (s,1H), 10.12 (s,1H), 7.51 (d, J=8.4 Hz, 2H), 7.34 (d, J=5.1 Hz, 1H), 7.17 (d, J=8.2 Hz, 2H), 6.92 (m,1H), 6.75-6.86 (m,3H), 6.64-6.74 (m,2H), 4.54 (d, J=15.1 Hz, 1H), 4.14 (d, J=15.1 Hz, 1H), 4.08 (t, J=6.3 Hz, 1H), 3.45 (s,2H), 3.43 (s,2H), 2.98-3.10 (m,2H). LRMS (ESI): (calc.) 481.6; (found) 482.4 (MH)⁺.

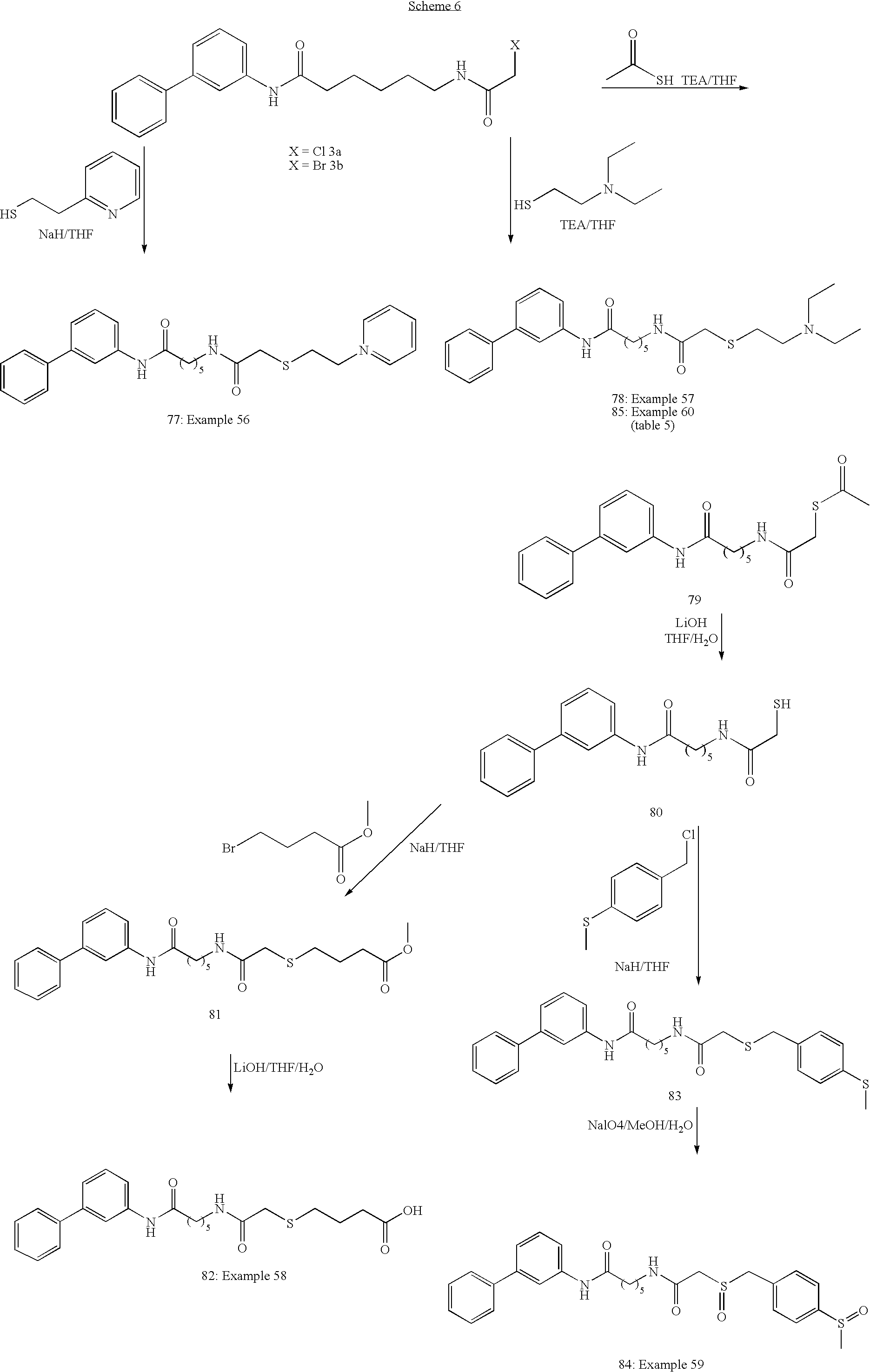

### EXAMPLE 56

### N-(biphenyl-3-yl)-6-(2-(2-(pyridin-2-yl)ethylthio)acetamido)hexanamide (77)

### Step 1: N-(biphenyl-3-yl)-6-(2-(2-(pyridin-2-yl)ethylthio)acetamido)hexanamide (77)

To a solution of 3a (108 mg, 0.302 mmol) in THF (3 mL) was added 2-(pyridin-2-yl)ethanethiol (50 mg, 0.333 mmol) and NaH (0.048 g, 1.21 mmol, 60% dispersion in oil). The resulting solution was stirred at room temperature for 2 h before being diluted with brine, basified to pH=10 with NaOH solution, and extracted with EtOAc. The organic extracts were dried (Na₂SO₄), filtered, and evaporated. The residue was purified by silica gel column chromatography with gradient of MeOH (0-25%) in AcOEt to afford 77 (44 mg, 32%) as a light yellow oil. (DMSO-d6) δ (ppm) ¹H, 10.18 (s,1H), 8.68 (s,1H), 8.22 (s,1H), 8.14 (s,1H), 7.94-7.84 (m,1H), 7.84-7.74 (m,3H), 7.72-7.63 (m,2H), 7.62-7.53 (m,2H), 7.54-7.43 (m,2H), 7.43-7.36 (m,1H), 3.60 (s,2H), 3.28 (m,2H), 3.24-3.10 (m,4H), 2.60-2.50 (m,2H), 1.90-1.76 (m,2H), 1.72-1.60 (m,2H), 1.60-1.48 (m,2H). LRMS (ESI): (calc.) 461.6; (found) 462.4 (MH)⁺.

### EXAMPLE 57

### N-(biphenyl-3-yl)-6-(2-(2-(diethylamino)ethylthio)acetamido)hexanamide (78)

### Step 1: N-(biphenyl-3-yl)-6-(2-(2-(diethylamino)ethylthio)acetamido)hexanamide (78)

Following the same procedure as described for compound 4 (step 4, scheme 1, example 1) but substituting 2-(diethylamino)ethanethiol for methyl 2-mercaptoacetate to afford 78 (48 mg, 35%) as a light yellow solid. ¹H NMR: (CDCl₃) δ (ppm): 7.92 (br s,1H), 7.82 (s,1H), 7.60-7.50 (m,3H), 7.44-7.28 (m,6H), 3.29 (q, J=13.1, 6.7 Hz, 2H), 3.23 (s,2H), 2.73-2.63 (m,4H), 2.58 (q, J=14.1, 7.0 Hz, 4H), 2.41 (t, J=7.2 Hz, 2H), 1.83-1.73 (m,2H), 1.64-1.54 (m,2H), 1.80-1.40 (m,2H), 1.04 (t, J=7.1 Hz, 6H). LRMS (ESI): (calc.) 455.7; (found) 456.3 (MH)⁺.

### EXAMPLE 58

### 4-(2-(6-(biphenyl-3-ylamino)-6-oxohexylamino)-2-oxoethylthio)butanoic acid (82)

### Step 1: S-2-(6-(biphenyl-3-ylamino)-6-oxohexylamino)-2-oxoethyl ethanethioate (79).

Following the same procedure as described for compound 4 (step 4, scheme 1, example 1) but substituting methyl 2-mercaptoacetate for thioacetic acid to afford 79 (383 mg, 96%) as a light grey solid. (DMSO-d6) δ (ppm) ¹H: 9.99 (s,1H), 8.12 (m,1H), 7.94 (s,1H), 7.65-7.57 (m,3H), 7.49 (t, J=7.8 Hz, 2H), 7.42-7.36 (m,2H), 7.35-7.31 (m,1H), 3.59 (s,2H), 3.37 (s,3H), 3.08 (q, J=12.7,6.7 Hz, 2H), 2.36 (m,2H), 1.69-1.59 (m,2H), 1.51-1.42 (m,2H), 1.40-1.30 (m,2H). LRMS (ESI): (calc.) 398.5; (found) 399.0 (MH)⁺.

### Step 2: N-(biphenyl-3-yl)-6-(2-mercaptoacetamido)hexanamide (80)

Following the same procedure as described for compound 5 (step 5, scheme 1, example 1) but substituting compound 79 for methyl ester 4 to afford 80 (323 mg, 94%) as a orange solid. (DMSO-d6) δ (ppm) ¹H, 9.99 (s,1H), 8.01 (m,1H), 7.94 (s,1H), 7.65-7.56 (m,3H), 7.49 (t, J=7.8 Hz, 2H), 7.43-7.36 (m,2H), 7.35-7.31 (m,1H), 3.14-3.08 (m,4H), 2.75 (t, J=7.8 Hz, 1H), 2.36 (t, J=7.4 Hz, 2H), 1.70-1.59 (m,2H), 1.53-1.42 (m,2H), 1.40-1.31 (m,2H). LRMS (ESI): (calc.) 356.5; (found) 357.2 (MH)⁺.

### Step 3: methyl 4-(2-(6-(biphenyl-3-ylamino)-6-oxohexylamino)-2-oxoethylthio)butanoate (81)

Following the same procedure as described for compound 77 (step 1, scheme 6, example 66) but substituting methyl 4-bromobutanoate for compound 3a and compound 80 for 2-(pyridin-2-yl)ethanethiol to afford 81 (40 mg, 22%) as a light yellow solid. (DMSO-d6) δ (ppm) ¹H: 9.98 (s,1H), 7.99 (m,1H), 7.94 (s,1H), 7.64-7.56 (m,3H), 7.49 (t, J=7.8 Hz, 2H), 7.40 (t, J=7.8 Hz, 2H), 7.35-7.31 (m,1H), 3.61 (s,3H), 3.11-3.07 (m,4H), 2.58 (t, J=7.2 Hz, 2H), 2.41 (t, J=7.4 Hz, 2H), 2.36 (t, J=7.24 Hz, 2H), 1.78-1.85 (m,2H), 1.70-1.60 (m,2H), 1.52-1.43 (m,2H), 1.40-1.30 (m,2H). LRMS (ESI): (calc.) 456.6; (found) 457.0 (MH)⁺.

### Step 4: 4-(2-(6-(biphenyl-3-ylamino)-6-oxohexylamino)-2-oxoethylthio)butanoic acid (82)

Following the same procedure as described for compound 5 (step 5, scheme 1, example 1) but substituting compound 81 for methyl ester 4 to afford 82 (15 mg, 61%) as a white solid. (DMSO-d6) δ (ppm) ¹H, 12.11 (br s,1H), 9.99 (s,1H), 8.00 (t, J=5.5 Hz, 1H), 7.95 (s,1H), 7.65-7.57 (m,3H), 7.49 (t, J=7.5 Hz, 2H), 7.43-7.36 (m,2H), 7.35-7.31 (m,1H), 3.14-3.06 (m,4H), 2.59 (t, J=7.2 Hz, 2H), 2.40-2.30 (m,4H), 1.84-1.73 (m,2H), 1.70-1.60 (m,2H), 1.54-1.42 (m,2H), 1.40-1.31 (m,2H). LRMS (ESI): (calc.) 442.6; (found) 443.4 (MH)⁺.

### EXAMPLE 59

### N-(biphenyl-3-yl)-6-(2-(4-(methylsulfinyl)benzylthio)acetamido)hexanamide (84)

### Step 3: N-(biphenyl-3-yl)-6-(2-(4-(methylthio)benzylthio)acetamido)hexanamide (83)

Following the same procedure as described for compound 77 (step 1, scheme 6, example 56) but substituting (4-thiomethyl)-benzyl chloride for compound 3a and compound 80 for 2-(pyridin-2-yl)ethanethiol to afford 83 (75 mg, 30%) as a light yellow solid. (DMSO-d6) δ (ppm) ¹H, 9.99 (s,1H), 7.98 (t, J=5.5 Hz, 1H), 7.94 (s,1H), 7.64-7.57 (m,3H), 7.52-7.46 (m,2H), 7.43-7.36 (m,2H), 7.35-7.31 (m,1H), 7.29-7.24 (m,2H), 7.24-7.19 (m,2H), 3.77 (s,2H), 3.09 (q, J=12.5,6.5 Hz, 2H), 3.01 (s,2H), 2.48 (s,3H), 2.37 (t, J=7.4 Hz, 2H), 1.70-1.60 (m,2H), 1.51-1.43 (m,2H), 1.41-1.30 (m,2H). LRMS (ESI): (calc.) 492.7; (found) 493.2 (MH)⁺.

### Step 4: N-(biphenyl-3-yl)-6-(2-(4-(methylsulfinyl)benzylthio)acetamido)hexanamide (84)

Following the same procedure as described for compound 7 (step 6, scheme 1, example 2) but substituting compound 83 for compound 5 to afford 84 (24 mg, 32%) as a white solid. (DMSO-d6) δ (ppm) ¹H, 10.32 (s,1H), 8.50 (m,1H), 8.00 (s,1H), 7.70 (d, J=8.0 Hz, 2H), 7.65 (d, J=7.6 Hz, 1H), 7.60 (d, J=7.4 Hz, 2H), 7.56-7.44 (m,4H), 7.41-7.35 (m,2H), 7.29-7.34 (m,1H), 4.36 (d, J=12.7 Hz, 1H), 4.27-4.20 (m,1H), 4.10 (d, J=12.9 Hz, 1H), 3.77 (d, J=13.3 Hz, 1H), 3.48 (d, J=11.4 Hz, 1H), 3.18 (d, J=5.3 Hz, 2H), 3.16-3.08 (m,2H), 2.79 (s,3H), 2.39 (t, J=7.0 Hz, 2H), 1.70-1.57 (m,2H), 1.54-1.42 (m,2H), 1.40-1.30 (m,2H). LRMS (ESI): (calc.) 524.7; (found) 525.1 (MH)⁺.

### EXAMPLE 60

Example 60 describes the preparation of compound 85 using the same procedures as described Example 57. Characterization data are presented in a Table 5.

TABLE 5

| Ex | Cpd | R | Name | Characterization | Scheme |
|---|---|---|---|---|---|
| 60 | 85 | 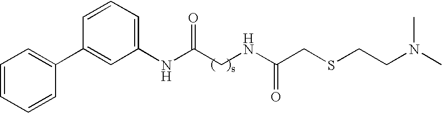 | N-(biphenyl-3-yl)- 6-(2-(2- (dimethylamino)- ethylthio)- acetamido)- hexanamide | ¹H NMR: (MeOD) δ (ppm): 7.87 (m, 1H), 7.64-7.60 (m, 2H), 7.57-7.53 (m, 1H), 7.48-7.43 (m, 2H), 7.42-7.32 (m, 3H), 3.26 (t, J=6.8 Hz, 2H), 3.21 (s, 2H), 2.79-2.73 (m, 2H), 2.69-2.63 (m, 2H), 2.45 (t, J=7.4 Hz, 2H), 2.35 (s, 6H), 1.84-1.74 (m, 2H), 1.67-1.57 (m, 2H), 1.53-1.43 (m, | |
| # 2H), 1.53-1.43 (m, 2H) LRMS (ESI): (calc.) 427.6; (found) 428.4 (MH)⁺. | 6 Step 1 Ex 57 | | | | |

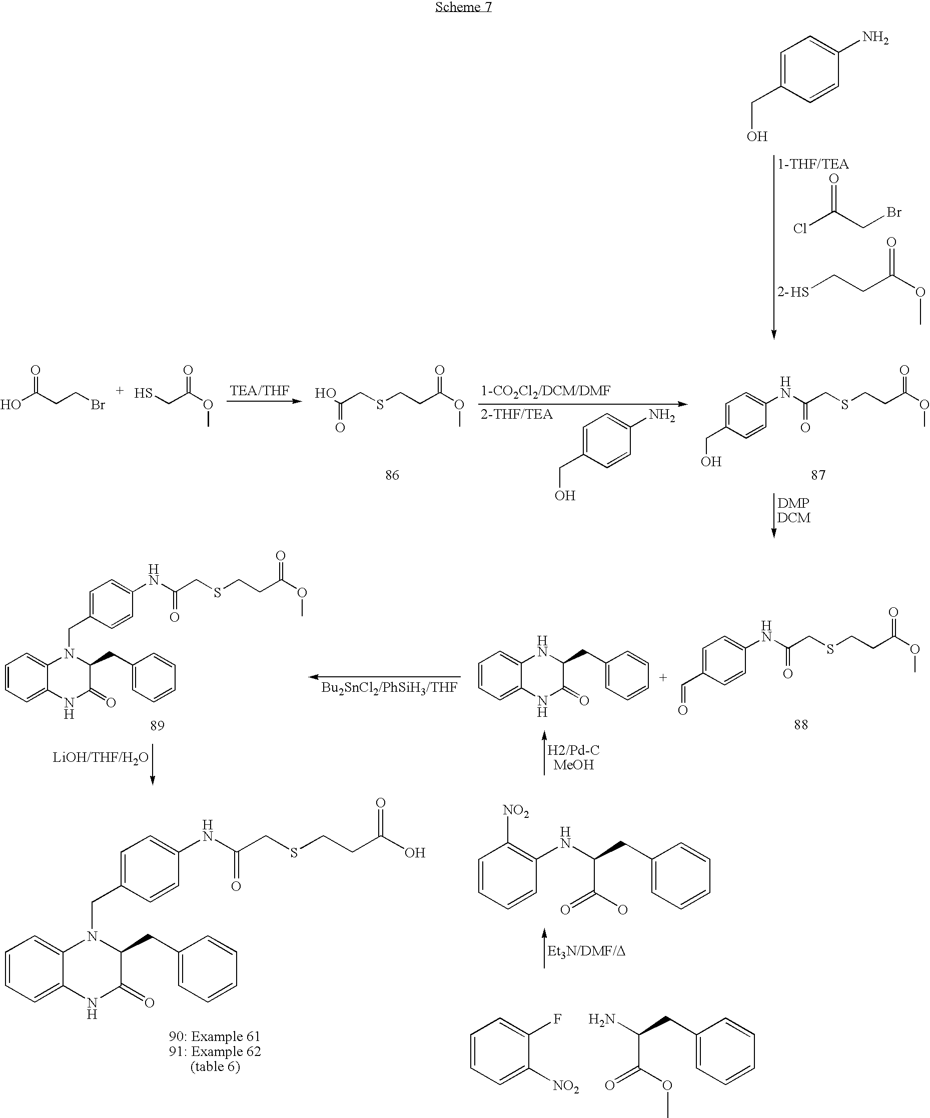

### EXAMPLE 61

### (S)-3-(2-(4-((2-benzyl-3-oxo-3,4-dihydroquinoxalin-1(2H)-yl)methyl)phenylamino)-2-oxoethylthio)propanoic acid (90)

### Procedure 1: methyl 3-(2-(4-(hydroxymethyl)phenylamino)-2-oxoethylthio)propanoate (87)

### Step 1: 2-(3-methoxy-3-oxopropylthio)acetic acid (86)

Following the same procedure as described for compound 4 (step 4, scheme 1, example 1) but substituting 3-bromopropanoic acid for compound 3b to afford 86 (942 mg, 88%) as a light yellow oil. LRMS (ESI): (calc.) 178.2; (found) 179.2 (MH)⁺.

### Step 2: methyl 3-(2-(4-(hydroxymethyl)phenylamino)-2-oxoethylthio)propanoate (87)

To a solution of 86 (665 mg, 3.73 mmol) in CH₂Cl₂ (4 mL) was added oxalyl chloride (2.24 mL, 4.48 mmol) and DMF (3 drops). The reaction was stirred for 30 mm at room temperature. To this mixture was added drop wise a solution of 4-amino-benzyl alcohol (3.73 mmol) in THF (10 mL) and triethylamine (12 mmol). The mixture was stirred at room temperature for another 15 min prior to dilution with brine, and extraction with EtOAc. The organic extracts were dried (Na₂SO₄), filtered, and evaporated. The residue was purified by silica gel column chromatography with gradient of EtOAc (25-100%) in Hexane to afford 87 (264 mg, 25%) as a light yellow foam. LRMS (ESI): (calc.) 283.2; (found) 284.2 (MH)⁺.

### Procedure 2: methyl 3-(2-(4-(hydroxymethyl)phenylamino)-2-oxoethylthio)propanoate (87)

### Step 1: methyl 3-(2-(4-(hydroxymethyl)phenylamino)-2-oxoethylthio)propanoate (87)

Following the same procedure as described for compound 69 (step 2-3, scheme 5, example 55) but substituting (4-aminophenyl)methanol for compound 67 to afford 87 (1.15 g, 45%) as a yellow oil. (DMSO-d6) δ (ppm) ¹H: 10.01 (s, 1H), 7.49 (d, J=8.6 Hz, 2H), 7.22 (d, J=8.2 Hz, 2H), 5.10 (t, J=5.7 Hz, 1H), 4.42 (d, J=5.7 Hz, 2H), 3.59 (s, 3H), 3.30 (s, 2H), 2.83 (t, J=7.2 Hz, 2H), 2.67 (t, J=7.0 Hz, 2H). LRMS (ESI): (calc.) 283.2; (found) 284.2 (MH)⁺.

### Step 3: methyl 3-(2-(4-formylphenylamino)-2-oxoethylthio)propanoate (88)

Following the same procedure as described for compound 71 (step 5, scheme 5, example 55) but substituting compound 87 for compound 70 to afford 88 (320 mg, 67%) as a white solid. (DMSO-d6) δ (ppm) ¹H: 10.49 (s, 1H), 9.85 (s, 1H), 7.85 (d, J=8.6 Hz, 2H), 7.77 (d, J=8.6 Hz, 2H), 3.58 (s, 3H), 3.37 (s, 2H), 2.84 (t, J=6.6 Hz, 2H), 2.67 (t, J=6.8 Hz, 2H).

### Synthesis of (S)-3-benzyl-3,4-dihydroquinoxalin-2(1H)-one. Step 1 and 2

### Step 1: (S)-methyl 2-(2-nitrophenylamino)-3-phenylpropanoate

To a solution of (S)-methyl 2-amino-3-phenylpropanoate (3.26 g, 23.2 mmol) and 1-fluoro-3-nitrobenzene (5 g, 23.2 mmol) in DMF (40 mL) were added triethyl amine (8.08 mL, 58.0 mmol). The reaction was heated at 100° C. for 16 hours and then the solvent was evaporated. EtOAc was added (30 mL), and the organic layer was washed with water. The organic extracts were dried (Na₂SO₄), filtered, and evaporated to afford the crude methyl ester as an orange gum. The crude was used in the next step without further purification.

### Step 2: (S)-3-benzyl-3,4-dihydroquinoxalin-2(1H)-one

Following the same procedure as described for compound 74 (step 3, scheme 5, example 55) but substituting (S)-methyl 2-(2-nitrophenylamino)-3-phenylpropanoate for 73 to afford (S)-3-benzyl-3,4-dihydroquinoxalin-2(1H)-one (130 mg, 12% after two steps) as a colourless oil. (DMSO-d6) δ (ppm) ¹H: 10.20 (s, 1H), 7.30-7.22 (m, 2H), 7.18-7.16 (m, 3H), 6.74-6.70 (m, 1H), 6.65 (d, J=7.7 Hz, 2H), 6.56-6.51 (m, 1H), 5.84 (s, 1H), 4.01-3.97 (m, 1H), 2.94-2.81 (m, 2H).

### Step 4: (S)-methyl 3-(2-(4-((2-benzyl-3-oxo-3,4-dihydroquinoxalin-1(2H)-yl)methyl)phenylamino)-2-oxoethylthio)propanoate (89)

Following the same procedure as described for compound 75 (step 6, scheme 5, example 55) but substituting (S)-3-benzyl-3,4-dihydroquinoxalin-2(1H)-one for compound 73 and compound 88 for 71 to afford 89 (90 mg, 36%) as a light yellow solid. (DMSO-d6) δ (ppm) ¹H: 10.41 (s, 1H), 10.04 (s, 1H), 7.47 (d, J=8.6 Hz, 2H), 7.22-7.13 (m, 3H), 7.09 (d, J=8.4 Hz, 2H), 7.05-7.03 (m, 2H), 6.82-6.74 (m, 2H), 6.67-6.63 (m, 2H), 4.40 (d, J=14.7 Hz, 1H), 4.01-3.97 (m, 2H), 3.57 (s, 3H), 3.28 (s, 2H), 2.83-2.75 (m, 3H), 2.70-2.64 (m, 3H). MS: 503.2 (calc) 504.2 (found)

### Step 5: (S)-3-(2-(4-((2-benzyl-3-oxo-3,4-dihydroquinoxalin-1(2H)-yl)methyl)phenylamino)-2-oxoethylthio)propanoic acid (90)

Following the same procedure as described for compound 5 (step 5, scheme 1, example 1) but substituting compound 89 for compound 4 to afford 90 (50 mg, 64%) as a yellow solid. (DMSO-d6) δ (ppm) ¹H, 12.22 (br s, 1H), 10.41 (s, 1H), 10.04 (s, 1H), 7.47 (d, J=8.4 Hz, 2H), 7.22-7.13 (m, 3H), 7.09 (d, J=8.0 Hz, 2H), 7.04 (d, J=7.0 Hz, 2H), 6.82-6.74 (m, 2H), 6.67-6.63 (m, 2H), 4.39 (d, J=14.7 Hz, 1H), 4.01-3.97 (m, 2H), 3.28 (s, 2H), 3.11-3.06 (m,1H), 2.78 (t, J=7.2 Hz, 2H), 2.70-2.66 (m,1H), 2.55 (t, J=6.9 Hz, 2H). LRMS (ESI): (calc.) 489.2; (found) 490.4 (MH)⁺.

### EXAMPLE 62

Example 62 describe the preparation of compound 91 using the same procedures as described for compound 90 in Example 61. Characterization data are presented in a Table 6.

TABLE 6

| Ex | Cpd | R | Name | Characterization | Scheme |
|---|---|---|---|---|---|
| 62 | 91 | 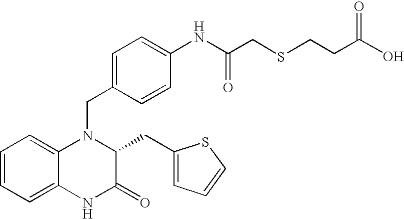 | (R)-3-(2-oxo-2- (4-((3-oxo-2- (thiophen-2- ylmethyl)-3,4- dihydroquinoxalin- 1(2H)-yl)methyl)- phenylamino-) ethylthio)-propanoic acid | (DMSO-d6) δ (ppm) ¹H: 10.24 (s, 1H), 10.09 (s, 1H), 7.20-7.40 (m, 2H), 6.80-7.10 (m, 3H), 6.25-6.70 (m, 6H), 4.26 (m, 1H), 3.84 (m, 2H), 3.05 (m, 2H), 2.78 (m, 2H), 2.16-2.34 (m, 2H), 1.76-1.86 (m, 2H) LRMS (ESI): (calc.) 495.6; | |
| # (found) 496.2 (MH)⁺. | 7 | | | | |

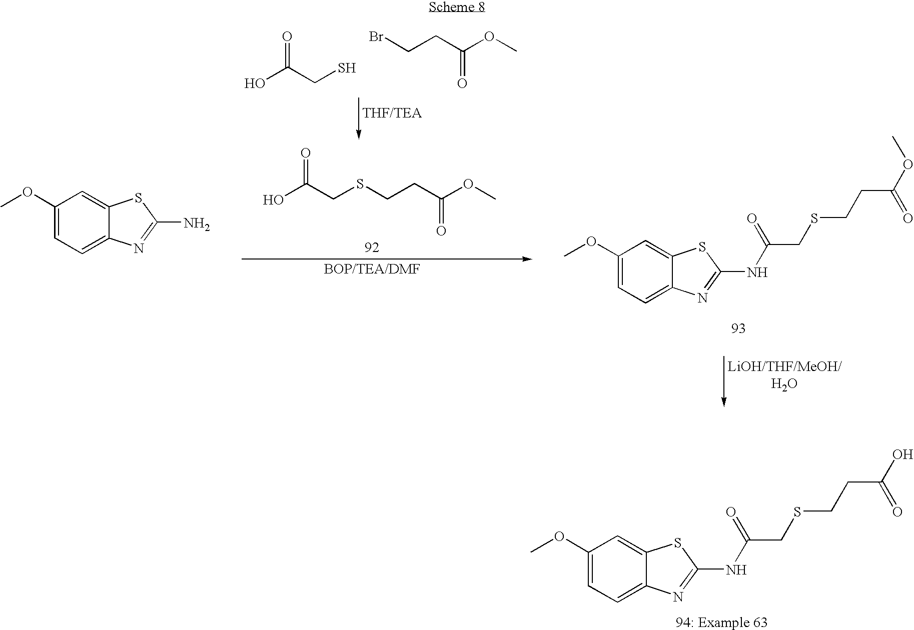

### EXAMPLE 63

### 3-(2-(6-methoxybenzo[d]thiazol-2-ylamino)-2-oxoethylthio)propanoic acid (94)

### Step 1: 2-(3-methoxy-3-oxopropylthio)acetic acid (92)

Following the same procedure as described for compound 4 (step 4, scheme 1, example 1) but substituting methyl 3-bromopropanoate for compound 3b and 2-mercaptoacetic acid for methyl 2-mercaptoacetate to afford 92 (10.1 mg, 95%) of the title compound as colorless oil.

### Step 1: methyl 3-(2-(6-methoxybenzo[d]thiazol-2-ylamino)-2-oxoethylthio)propanoate (93)

Following the same procedure as described for compound 1 (step 1, scheme 1, example 1) but substituting 6-methoxybenzo[d]thiazol-2-amine for biphenyl-3-amine and using acid 92 to afford 93 (250 mg, 51%) as white solid.

### Step 2: 3-(2-(6-methoxybenzo[d]thiazol-2-ylamino)-2-oxoethylthio)propanoic acid (94)

Following the same procedure as described for compound 5 (step 5, scheme 1, example 1) but substituting compound 93 for compound 4 to afford 94 (30 mg, 13%) as a white solid. (DMSO-d6) δ (ppm) ¹H: 12.31 (br s,1H), 7.62 (d, J=8.7 Hz, 1H), 7.56 (d, J=2.3 Hz, 1H), 7.01 (dd, J=8.8, 2.5 Hz, 1H), 3.80 (s, 3H), 3.46 (s, 2H), 2.80 (t, J=7.2 Hz, 2H), 2.57 (t, J=6.9 Hz, 2H).

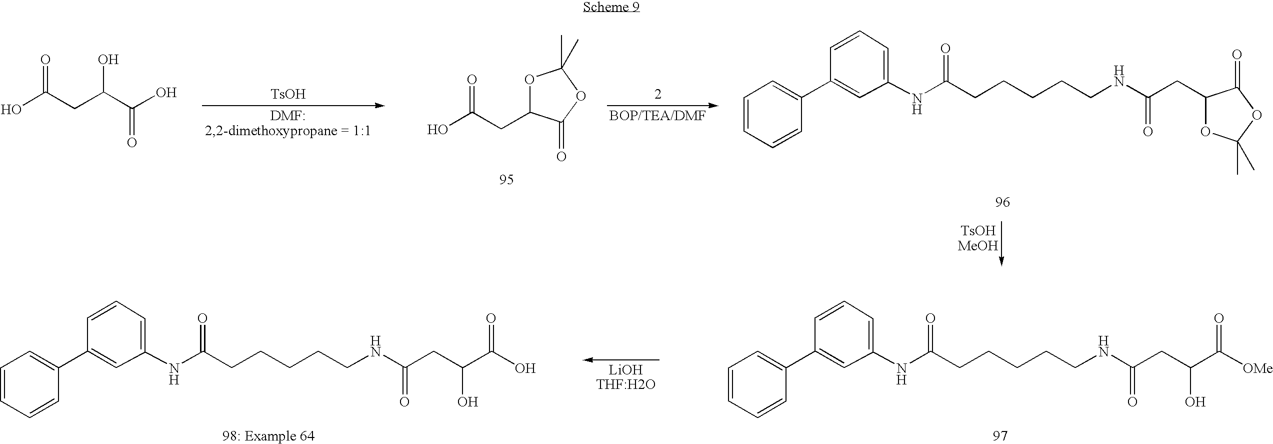

### EXAMPLE 64

### 4-(6-(biphenyl-3-ylamino)-6-oxohexylamino)-2-hydroxy-4-oxobutanoic acid (98)

### Step 1: 2-(2,2-dimethyl-5-oxo-1,3-dioxolan-4-yl)acetic acid (95)

To a solution of DL-malic acid (4 g, 29.8 mmol) in DMF: 2,2′-dimethoxypropane (1:1, 35 ml) was added p-TsOH (285 mg, 1.5 mmol). The reaction was stirred for 4 h at room temperature. EtOAc was added, and the organic layer was washed with 2M aqueous HCl (pH=2). The organic extracts were dried (Na₂SO₄), filtered, and evaporated to afford 95 (5.2 g, 99%) as a white solid. LRMS (ESI): (calc.) 174.05; (found) 197.00 (MNa)⁺.

### Step 2: N-(biphenyl-3-yl)-6-(2-(2,2-dimethyl-5-oxo-1,3-dioxolan-4-yl)acetamido)hexanamide (96)

Following the same procedure as described for compound 1 (step 1, scheme 1, example 1) but substituting compound 2 for biphenyl-3-amine and using acid 95 to afford 96 (87 mg, 63%) as a clear translucent oil. LRMS (ESI): (calc.) 438.22; (found) 461.51 (MNa)⁺.

### Step 3: methyl 4-(6-(biphenyl-3-ylamino)-6-oxohexylamino)-2-hydroxy-4-oxobutanoate (97)

To a solution of 96 (87 mg) in methanol was added p-TsOH (86 mg, 0.2 mmol). The reaction was stirred 3 h at room temperature and the concentrated in vacuo. The residue was purified by silica gel column chromatography with gradient of MeOH (50-90%) in AcOEt and then MeOH (10%) in EtOAc to afford 97 (28 mg, 35%) as a clear translucent oil. (CDCl₃) δ (ppm) ¹H: 8.08-8.01(m,1H), 7.81(s,1H), 7.56-7.50(m,3H), 7.41-7.29(m,5H), 6.51(s,1H), 4.49(s, 1H), 3.73(s,3H), 3.67(s,1H), 3.23(m,2H), 2.74-2.61 (m,2H), 2.38(t,2H,J=7 Hz), 1.72(2,H), 1.52(s,2H), 1.37(m,2H). LRMS (ESI): (calc.) 412.2; (found) 413.3 (MH)⁺.

### Step 4: 4-(6-(biphenyl-3-ylamino)-6-oxohexylamino)-2-hydroxy-4-oxobutanoic acid (98)

Following the same procedure as described for compound 5 (step 5, scheme 1, example 1) but substituting compound 97 for compound 4 to afford 98 (25 mg, 99%) as a white solid. (acetone-d6) δ(ppm) ¹H, 9.05(s,1H), 7.86(s,1H), 7.50-Hz), 2.73(s,1H), 2.49(m,2H), 2.26(t,2H,J=7 Hz), 1.59(m,2H), 1.42(m,2H), 1.27(m,2H). LRMS (ESI): (calc.) 398.18; (found) 399.2 (MH)⁺.

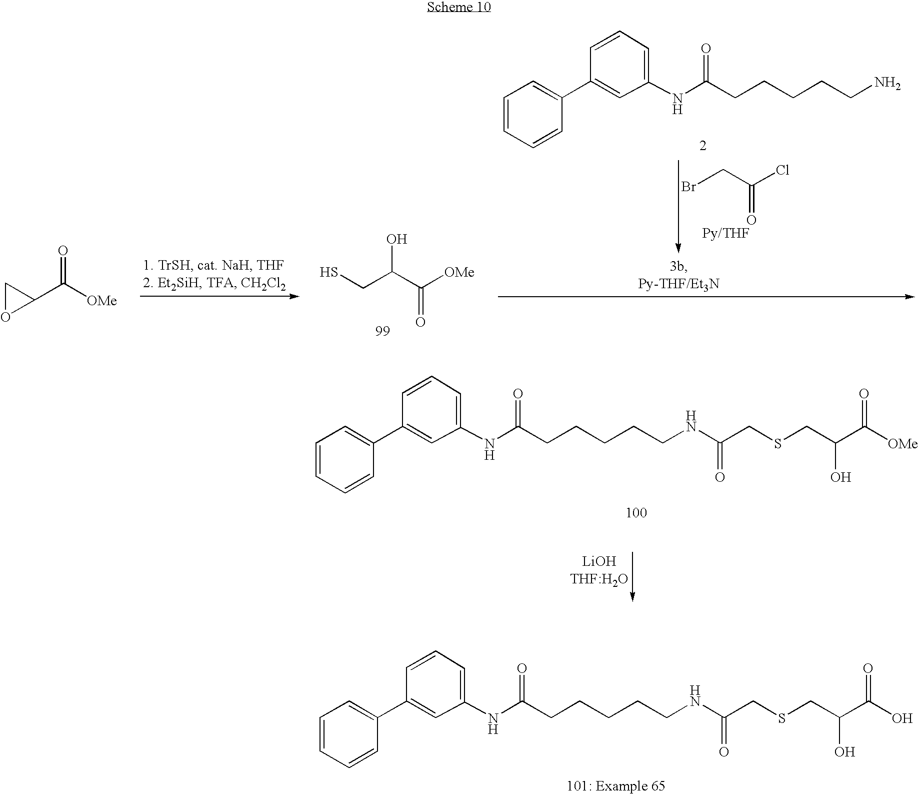

### EXAMPLE 65

### 3-(2-(6-(biphenyl-3-ylamino)-6-oxohexylamino)-2-oxoethylthio)-2-hydroxypropanoic acid (101)

### Step 1: methyl 2-hydroxy-3-mercaptopropanoate (99)

To a suspension of triphenylmethane thiol (1.35 g, 4.9 mmol) and NaH (20 mg, 0.49 mmol) in THF (20 ml) was slowly added methyl glycidyl ester (500 mg, 4.9 mmol). The reaction was stirred at 0° C. and then warmed to 23° C. over 20 minutes. The reaction mixture was quenched with sat. aqueous NH₄Cl (20 ml) and extracted with ethyl acetate. The organic extract was dried (Na₂SO₄), filtered, and evaporated. The residue was purified by silica gel column chromatography with gradient of EtOAc (10-20%) in hexane to afford the desired compound (950 mg, 51%) as a clear translucent oil. LRMS (ESI): (calc.) 378.13; (found) 401.29 (MH)⁺.

To a solution of ester (950 mg, 2.5 mmol) and Et₃SiH (441 μl, 2.76 mmol) in CH₂Cl₂ (12 ml) was added TFA (970 μl, 5 equiv, 12.6 mmol). The reaction was stirred for 30 minutes at room temperature. The reaction mixture was quenched with NaHCO₃ (ss) (10 ml) and extracted with CH₂Cl₂. The organic extract was dried (Na₂SO₄), filtered, and evaporated. The residue was purified by silica gel column chromatography with gradient of EtOAc (10-50%) in hexane to afford 99 (299 mg, 87%) as a clear translucent oil. LRMS (ESI): (calc.) 136.2; (found) (MH)⁺.

### Step 2: methyl 3-(2-(6-(biphenyl-3-ylamino)-6-oxohexylamino)-2-oxoethylthio)-2-hydroxypropanoate (100)

To a solution of 2 (100 mg, 0.35 mmol) in pyridine (71 mL):THF (1 ml) was added bromoacetyl chloride (29 ml, 0.35 mmol). The reaction was stirred at 0° C. and then warmed to 23° C. over 15 minutes. After the mixture was cooled back to 0° C. followed by the sequential addition of methyl 3-mercapto-2-hydroxy propionate (48 mg, 0.35 mmol) and Et₃N (123 ml, 0.89 mmol). The reaction was stirred for another 20 minutes at room temperature and extracted from brine with EtOAc. The organic extract was dried (Na₂SO₄), filtered, and evaporated. The residue was purified by silica gel column chromatography with gradient of EtOAc (50-100%) in hexane to afford 100 (36 mg, 22%) as a clear translucent oil. (acetone-d6) δ (ppm) ¹H: 9.20(s,1H), 8.01(s,1H), 7.63-7.61(m,3H), 7.47-7.43(m,3H), 7.37-7.33(m,3H), 4.41(dd,1H,J=4,6 Hz), 3.70(s,3H), 3.28-3.22(m,2H), 3.03(dd,1H,J=6,14 Hz), 2.93(dd,1H,J=6,14 Hz), 2.87(d,1H,J=12 Hz), 2.42(t,2H,J=7 Hz), 1.73(m,2H), 1.57(m,2H), 1.43(m,2H). LRMS (ESI): (calc.) 458.1; (found) 459.5 (MH⁺).

### Step 3: 3-(2-(6-(biphenyl-3-ylamino)-6-oxohexylamino)-2-oxoethylthio)-2-hydroxypropanoic acid (101)

Following the same procedure as described for compound 5 (step 5, scheme 1, example 1) but substituting compound 100 for compound 4 to afford 101 (34 mg, 98%) as a solid. (MeOD-d4) δ (ppm) ¹H: 7.85(s,1H), 7.58-7.52(m,3H), 7.42-7.34(m,5H), 4.29(m, 1H), 3.31-3.24(m,5H), 3.03-2.99(dd, 1H,J=5,14 Hz), 2.87-2.83(dd,1H,J=5,14 Hz), 2.43(m,2H), 1.88(m,2H), 1.61(m,2H), 1.46-1.41(m,2H). LRMS (ESI): (calc.) 444.1; (found) 451.5 (MLi⁺).

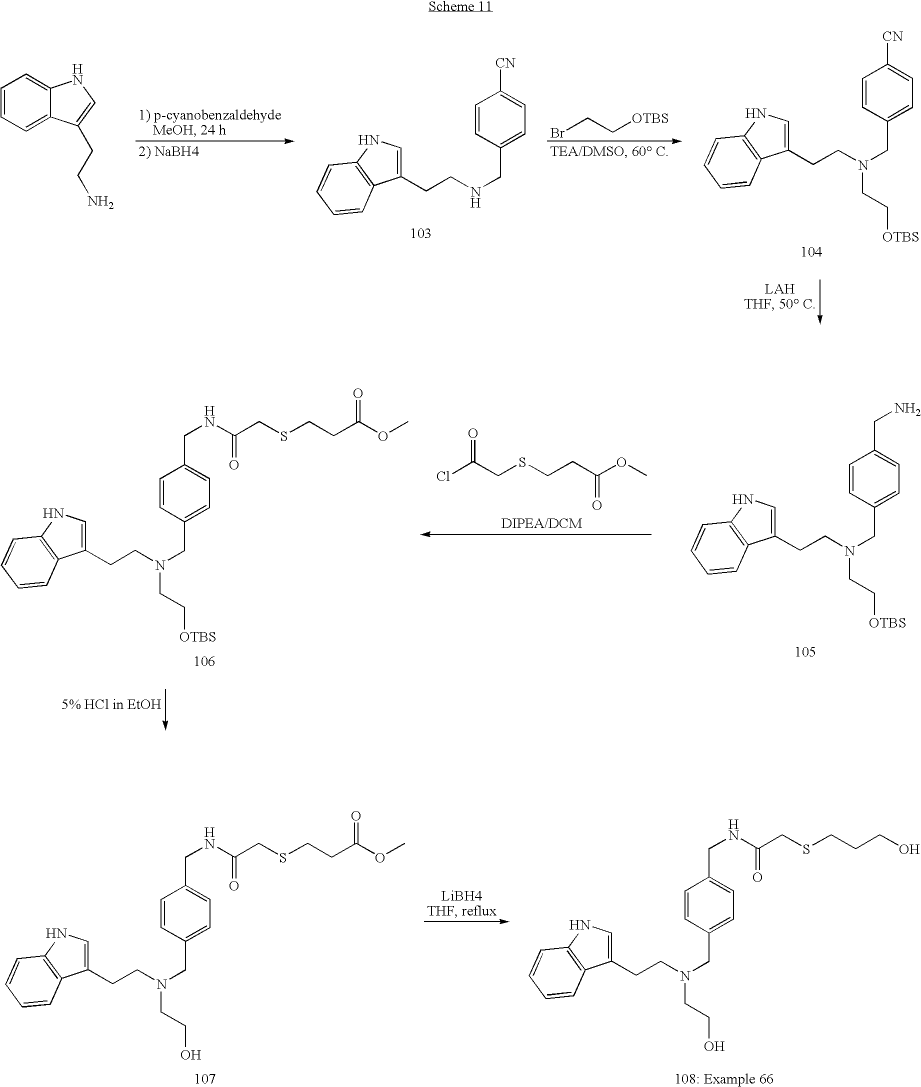

### EXAMPLE 66

### N-(4-(((2-(1H-indol-3-yl)ethyl)(2-hydroxyethyl)amino)methyl)benzyl)-2-(3-hydroxypropylthio)acetamide (108)

### Step 1: 4-((2-(1H-indol-3-yl)ethylamino)methyl)benzonitrile (103)

To a solution of tryptamine (1.02 g, 6.28 mmol) in 25 mL of MeOH was added p-cyanobenzaldehyde (841 mg, 6.41 mmol). The mixture was stirred at room temperature for 60 h. Sodium borohydride (238 mg, 6.28 mmol) was added and then the solution was stirred for another 1 h at room temperature. The reaction mixture was quenched with aqueous NH₄Cl (ss) (20 ml) and extracted with ethyl acetate. The organic extracts were washed with brine, dried (Na₂SO₄), filtered, and evaporated to give 103 in quantitative yield as a brown oil which was used for the next step without further purification. LRMS (ESI): (calc) 275.1 (found) 276.2 (MH⁺).

### Step 2: 4-((2-(1H-indol-3-yl)ethyl)(2-(tert-butyldimethylsilyloxy)ethyl)amino)methyl)benzonitrile (104)

To a crude mixture of 103 (1.73 g, 6.28 mmol) in DMSO (10 mL) was added DIPEA (1.40 mL, 8.00 mmol) and (2-bromoethoxy)-tert-butyldimethylsilane (1.67 g, 7.00 mmol). The reaction was stirred at 55° C. during 48 h. The mixture was quenched with water and extracted with DCM. The organic extracts were washed with water, dried (Na₂SO₄), filtered, and evaporated. The residue was purified by silica gel column chromatography with EtOAc (40%) in hexane to afford 104 (2.09 g, 77% after two steps) as a solid. LRMS (ESI): (calc.) 433.3; (found) 434.4(MH)⁺.

### Step 3: N-(2-(1H-indol-3-yl)ethyl)-N-(4-(aminomethyl)benzyl)-2-(tert-butyldimethylsilyloxy)ethanamine (105)

To a solution of 104 (690 mg, 1.59 mmol) in THF (15 mL) was added LAH (0.121 g, 3.19 mmol). The mixture was stirred for 1 h at 50° C. The mixture was quenched with aqueous solution of sodium sulfate (ss), filtered through a pad of celite and extracted with DCM. The organic extract was dried (Na₂SO₄), filtered, and evaporated. The residue was purified by silica gel column chromatography with MeOH (15%) in DCM with 1% of ammonium hydroxide to afford 105 (340 mg, 49%) as a yellow oil. LRMS (ESI): (calc.) 437.29; (found) 438.4 (MH)⁺.

### Step 4: methyl3-(2-(4-(((2-(1H-indol-3-yl)ethyl)(2-(tert-butyldimethylsilyloxy)ethyl)amino)methyl)benzylamino)-2-oxoothylthio)propanoate (106)

To a solution of 105 (340 mg, 0.778 mmol) in DCM (5 mL) was added DIPEA (0.23 mL, 1.3 mmol) and 3-chlorocarbonylmethylsulfanyl-propionic acid methyl ester (0.196 g, 1.0 mmol) in DCM (2 mL). The reaction mixture was stirred at room temperature for 5 minutes, then poured into a Na(HCO₃) (ss) and extracted with DCM. The organic extracts were washed with water, dried (Na₂SO₄), filtered, and evaporated to afford 106 (471 mg, 100%) which was used for the next step without purification. LRMS (ESI): (calc.) 597.3; (found) 598.5 (MH)⁺.

### Step 5: methyl3-(2-(4-(((2-(1H-indol-3-yl)ethyl)(2-hydroxyethyl)amino)methyl)benzylamino)-2-oxoethylthio)propanoate (107)

To a solution of 106 (465 mg, 0.778 mmol) in ethanol (5 mL) was added HCl (1 mL, 5%). The mixture was stirred 1 hour a room temperature. The reaction mixture was diluted in EtOAc and washed with Na(HCO₃) (ss), water and brine. The organic extract was dried (Na₂SO₄), filtered, and evaporated. The residue was purified by silica gel column chromatography with MeOH (15%) in DCM to afford 107 (0.252 g, 67%). LRMS (ESI): (calc.) 483.2; (found) 484.4 (MH)⁺.

### Step 6: N-(4-(((2-(1H-indol-3-yl)ethyl)(2-hydroxyethyl)amino)methyl)benzyl)-2-(3-hydroxypropylthio)acetamide (108)

Following the same procedure as described for compound 5 (step 5, scheme 1, example 1) but substituting compound 107 for compound 4 to afford 108 (39 mg, 11%) as a solid. (MeOD-d4) δ (ppm) ¹H: 7.42-7.38 (m, 3H), 7.34-7.32 (m, 3H), 7.11-7.08 (m, 2H), 7.00-6.96 (m, 1H), 4.40 (s, 2H), 4.29 (s, 2H), 3.87-3.84 (m, 2H), 3.60 (t, J=5.6 Hz, 2H), 3.36-3.30 (m, 2H, overlap with MeOH), 3.28-3.15 (m, 6H), 2.66 (t, J=7.2 Hz, 2H), 1.82-1.75 (m, 2H). LRMS (ESI): (calc.) 455.2; (found) 456.4 (MH)⁺.

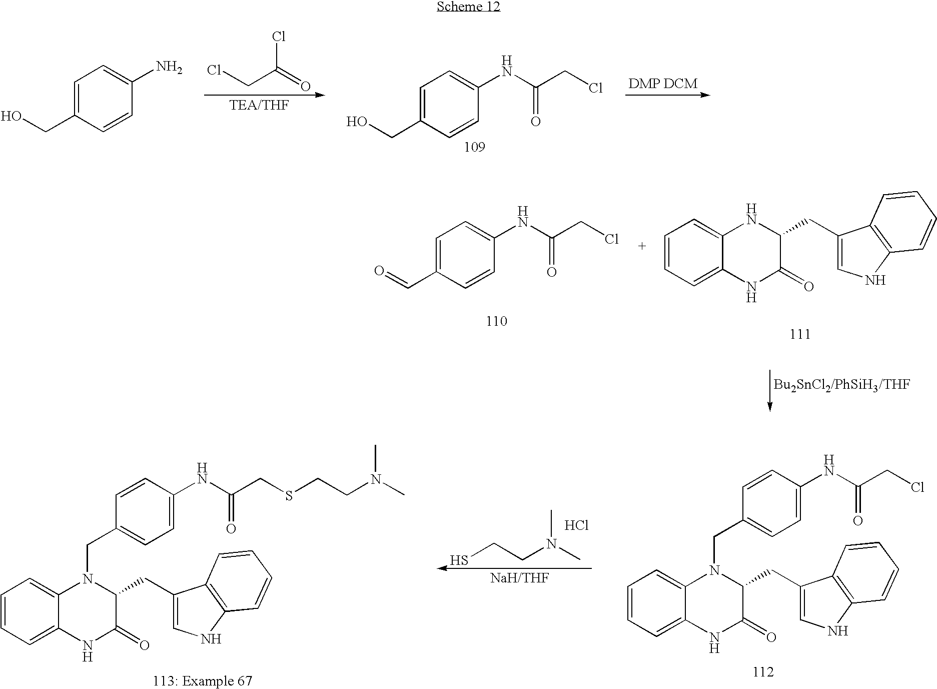

### EXAMPLE 67

### (R)-N-(4-((2-((1H-indol-3-yl)methyl)-3-oxo-3,4-dihydroquinoxalin-1(2H)-yl)methyl)phenyl)-2-(2-(dimethylamino)ethylthio)acetamide (113)

### Step 1: 2-chloro-N-(4-(hydroxymethyl)phenyl)acetamide (109)

Following the same procedure as described for compound 68 (step 2, scheme 5, example 55) but substituting (4-aminophenyl)methanol for compound 67 and 2-chloroacetyl chloride for 2-bromoacetyl chloride to afford 109 (850 mg, 66%) as a yellow solid. (DMSO-d6) δ (ppm) ¹H, 10.23 (s,1H), 7.51 (d, J=8.4 Hz, 2H), 7.25 (d, J=8.1 Hz, 2H), 5.11 (t, J=5.5 Hz, 1H), 4.43 (d, J=4.9 Hz, 2H), 4.23 (s, 2H).

### Step 2: 2-chloro-N-(4-formylphenyl)acetamide (110)

Following the same procedure as described for compound 65 (step 5, scheme 5, example 44) but substituting compound 109 for 70 to afford 110 (380 mg, 86%) as a yellow solid. (DMSO-d6) δ (ppm): 10.68 (s, 1H), 9.87 (s, 1H), 7.87 (d, J=8.6 Hz, 2H), 7.78 (d, J=8.5 Hz, 2H), 4.31 (s, 2H).

### Synthesis (R)-3-((1H-indol-3-yl)methyl)-3,4-dihydroquinoxalin-2(1H)-one (111)

Following the same procedure as described for (S)-3-benzyl-3,4-dihydroquinoxalin-2(1H)-one (step 1 and 2, scheme 7, example 61) but substituting (R)-methyl 2-amino-3-(1H-indol-3-yl)propanoate for (S)-methyl 2-amino-3-phenylpropanoate to afford 111 (550 mg, 51%) as a clear oil. (DMSO-d6) δ (ppm): 10.84 (s, 1H), 10.19 (s, 1H), 7.48 (d, J=8.0 Hz, 1H), 7.10 (s, 1H), 7.04 (td, J=7.0, 1.0 Hz, 1H), 6.94 (td, J=7.0, 1.0 Hz, 1H), 6.73-6.63 (m, 3H), 6.56-6.51 (m, 1H), 5.74 (s, 1H), 4.01-3.99 (m, 1H), 3.10-2.89 (m, 2H).

### Step 3: (R)-N-(4-((2-((1H-indol-3-yl)methyl)-3-oxo-3,4-dihydroquinoxalin-1(2H)-yl)methyl)phenyl)-2-chloroacetamide (112)

Following the same procedure as described for compound 75 (step 6, scheme 5, example 55) but substituting compound 110 for 71 and aniline 111 for 74 to afford 112 (230 mg, 40%) as a colourless oil. LRMS (ESI): (calc.) 458.2; (found) 459.2 (MH)⁺.

### Step 4: (R)-N-(4-((2-((1H-indol-3-yl)methyl)-3-oxo-3,4-dihydroquinoxalin-1(2H)-yl)methyl)phenyl)-2-(2-(dimethylamino)ethylthio)acetamide (113)

Following the same procedure as described for compound 77 (step 1, scheme 6, example 56) but substituting compound 112 for compound 3a and 2-(dimethylamino)ethanethiol hydrochloride for 2-(pyridin-2-yl)ethanethiol to afford 113 (20 mg, 16%) as a white solid. (MeOD-d4) δ(ppm) ¹H, 7.38-7.32 (m, 4H), 7.09 (t, J=7.1 Hz, 1H), 6.99-6.80 (m, 6H), 6.76-6.71 (m, 2H), 4.36 (d, J=14.7 Hz, 1H), 4.11-4.07 (m, 1H), 3.72 (d, J=14.8 Hz, 1H), 3.36 (s, 2H), 2.98-2.93 (m, 2H), 2.87-2.81 (m, 4H), 2.45 (s, 6H). LRMS (ESI): (calc.) 527.2; (found) 528.3 (MH)⁺.

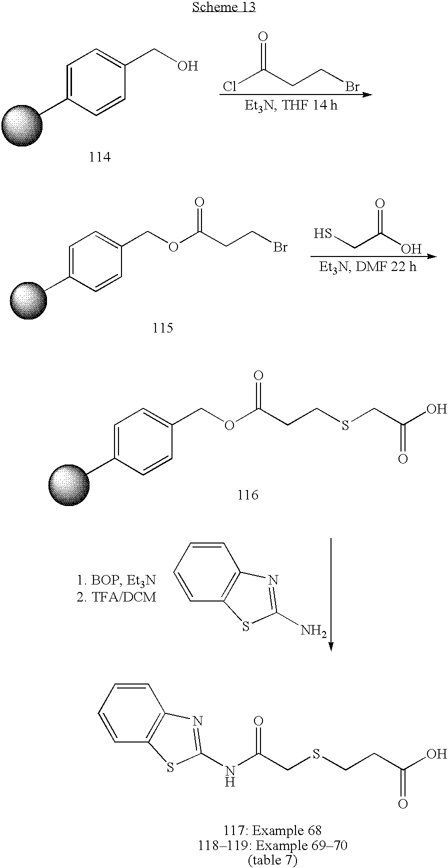

### EXAMPLE 68

### 3-(2-(benzo[d]thiazol-2-ylamino)-2-oxoethylthio)propanoic acid (117)

### Step 1: polymer supported 3-bromopropionate (115)

Wang resin 114 (3 g, 5.1 mmol, loading 1.7 mmol/g) was washed with dry THF, and then added 3-bromopropanoyl chloride (2.57 mL, 25.5 mmol), and triethylamine (3.55 mL, 25.5 mmol) in THF. The reaction was mechanically stirred gently for 14 h at room temp. The mixture was filtered and washed with DMF (×2), MeOH, DMF (×2), MeOH, DMF, THF, MeOH. The resin was dried under N₂ then under vacuum overnight to afford 115 (3.87 g, 80%).

### Step 2: polymer supported 2-(3-(benzyloxy)-3-oxopropylthio)acetic acid (116)

The resin 115 (3.87 g, 20.4 mmol) was swollen in DMF (100 ml), and then added 2-mercaptoacetic acid (7.1 ml, 102 mmol) and triethylamine (28.4 ml, 204 mmol). The misture was stirred gently at room temp for 22 h. The resin was filtered and washed with DMF (×5), 5% AcOH in DCM (×5), MeOH, DMF (×5), DCM (×5), and diethyl ether (×2) and dried under vacuum to afford 116 in quantitative yield.

### Step 3: 3-(2-(benzo[d]thiazol-2-ylamino)-2-oxoethylthio)propanoic acid (117)

To resin 116 (500 mg, 0.3 mmol) in DMF (5 ml) was added BOP (402 mg, 0.9 mmol), triethylamine (169 uL, 1.2 mmol). The mixture was stirred for 35 minutes, and then added 2-aminobenzothiazole (273 mg, 1.8 mmol). The reaction was stirred for another 16 h. The resin was filtered and washed exhaustively with DMF, 5% AcOH/DCM, MeOH, DMF, DCM, MeOH, then with DCM. The resin was then treated with 1:1 mixture of trifluoroacetic acid and dichloromethane for 3 h, and the filtrate was concentrated to afford 117 (5 mg, 5.5%) as beige solid after trituration from MeOH/H₂O and then from ether. (DMSO-d6) δ (ppm) ¹H: 7.96 (d, J=7.6 Hz, 1H), 7.73 (d, J=8.4 Hz, 1H), 7.42 (t, J=7.0 Hz, 1H), 7.30 (t, J=7.2 Hz, 1H), 3.49 (s, 2H), 2.81 (t, J=7.2 Hz, 2H), 2.57 (t, J=7.2 Hz, 2H). LRMS (ESI): (calc.) 296.03; (found) 297.1 (MH)⁺.

### EXAMPLE 69-70

Example 69-70 describe the preparation of compound 118-119 using the same procedures as described for compound 117 in Example 68. Characterization data are presented in a Table 7.

TABLE 7

| Ex | Cpd | Structure | Name | Characterization | Scheme |
|---|---|---|---|---|---|
| 69 | 118 | 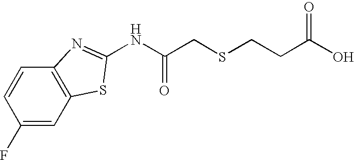 | 3-(2-(6- fluorobenzo[d]- thiazol-2-ylamino)- 2-oxoethylthio)- propanoic acid | (DMSO-d6) δ (ppm) ¹H: 12.45 (bs, 1H), 12.28 (bs, 1H), 7.88 (dd, J1=8.8 Hz, J2=2.4 Hz, 1H), 7.74 (q, J=4.5 Hz, 1H), 7.28 (td, J1=8.8 Hz, J2=2.4 Hz, 1H), 3.48 (s, 2H), 2.80 (t, J=7.2 Hz, 2H), 2.57 (t, J=7.2 Hz, 2H). LRMS (ESI): (calc.) 314.02; (found) 315.1 (MH)⁺. | 13 Step 1-3 |
| 70 | 119 | 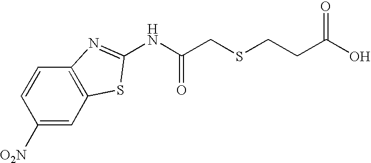 | 3-(2-(6- nitrobenzo[d]- thiazol-2-ylamino)- 2-oxoethylthio)- propanoic acid | (DMSO-d6) δ (ppm) ¹H: 12.88 (s, 1H), 12.28 (bs, 1H), 9.05 (d, J=2.4 Hz, 1H), 8.27 (dd, J1=9.0 Hz, J2=2.6 Hz, 1H), 7.89 (d, J=9.2 Hz, 1H), 3.53 (s, 2H), 2.81 (t, J=7.2 Hz, 2H), 2.57 (t, J=7.0 Hz, 2H). LRMS (ESI): (calc.) 341.0; (found) 342.0 (MH)⁺. | 13 Step 1-3 |

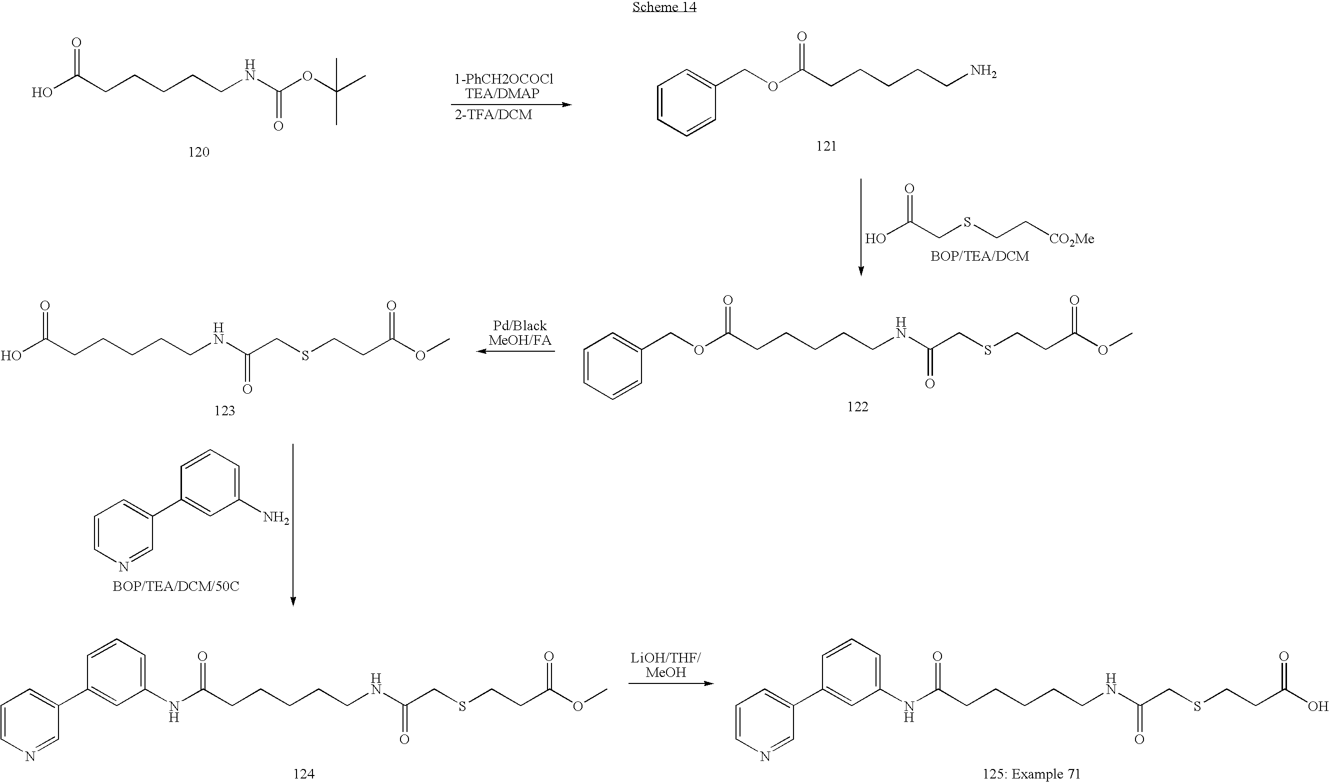

### EXAMPLE 71

### 3-(2-oxo-2-(6-oxo-6-(3-(pyridin-3-yl)phenylamino)hexylamino)ethylthio)-propanoic acid (125)

### Step 1: benzyl 6-aminohexanoate (121)

To a solution of 120 (5.0 g, 22 mmol) in DCM was added benzyl chloroformate (3.7 g, 3.34 mL, 22 mmol) at 0° C. followed by triethylamine (9 ml, 65 mmol) and DMAP (cat. amount). The mixture was stirred at room temperature for 4 hour and then the solvent was evaporated. EtOAc was added, and the organic layer was washed with NaHCO₃ (ss) and brine. The organic extracts were dried (MgSO₄), filtered, and evaporated. The residue was purified by silica gel column chromatography with gradient of EtOAc (20-50%) in hexanes to afford the desired compound (3.2 g, 50%) as colorless oil.

The Boc analog (3.2 g, 11 mmol) was treated with excess TFA. The reaction was stirred for 18 h at room temp and 121 was obtained in quantitative yield LRMS (ESI): (calc.) 221.3; (found) 222 (MH)⁺.

### Step 2: benzyl 6-(2-(3-methoxy-3-oxopropylthio)acetamido)hexanoate (122)

To a solution of 2-(3-methoxy-3-oxopropylthio)acetic acid (0.322 g, 2 mmol) in DCM was added triethylamine (1.0 mL, 4 mmol) and BOP (0.8 g, 2 mmol). The mixture was stirred for 15 minutes at room temperature, and then 121 was added (0.4 g, 2 mmol) and triethylamine (1.0 mL, 4 mmol). The mixture was heated at 50° C. for 16 h, and then the solvent was evaporated. EtOAc was added, and the organic layer was washed with water, NaHCO₃ (ss) and brine. The organic extracts were dried (MgSO₄), filtered, and evaporated. The residue was purified by silica gel column chromatography to afford 122 (0.17 g, 25%) as an oil. LRMS (ESI): (calc.) 381.5; (found) 382.3 (MH)⁺.

### Step 3: 6-(2-(3-methoxy-3-oxopropylthio)acetamido)hexanoic acid (123)

To a solution of 122 (0.17 g, 0.45 mmol) in MeOH:formic acid (1:1) (10 mL each) was added palladium black (0.17 g, 1 equivalent). The mixture was stirred for 16 h and then filtered over Celite to afford 118 (0.11 g, 84%).

### Step 4: methyl 3-(2-oxo-2-(6-oxo-6-(3-(pyridin-3-yl)phenylamino)hexylamino)-ethylthio)propanoate (124)

Following the same procedure as described for compound 122 (step 2, scheme 14, example 71) but substituting 3-(pyridin-3-yl)benzenamine for compound 121 and 123 for 2-(3-methoxy-3-oxopropylthio)acetic acid to afford 124 (20 mg, 41%) as a colorless oil. LRMS (ESI): (calc.) 443.6; (found) 444 (MH)⁺.

### Step 5: 3-(2-oxo-2-(6-oxo-6-(3-(pyridin-3-yl)phenylamino)hexylamino)ethylthio)-propanoic acid (125)

Following the same procedure as described for compound 5 (step 5, scheme 1, example 1) but substituting compound 124 for compound 4 to obtain the crude acid. The residue was purified by preparative HPLC (Aquasil C-18, 100×4.6, 5 uM) with MeOH in H₂O to afford 125 (1.5 mg, 3%) as a colorless oil.

(actone-d6) δ (ppm) ¹H, 8.8(s, 1H); 8.6(m, 1H); 8-8.2(m, 2H); 7.8(m, 1H); 7.6(m, 1H); 7.4(m, 2H); 3.3(m, 2H); 3.2(s, 2H); 2.6(dd, J=6 Hz, 2H); 2.4(dd, J=6 Hz, 2H); 1.7(m, 2H); 1.6(m, 2H); 1.4(m, 2H). LRMS (ESI): (calc.) 429.5; (found) 430.2 (MH)⁺.

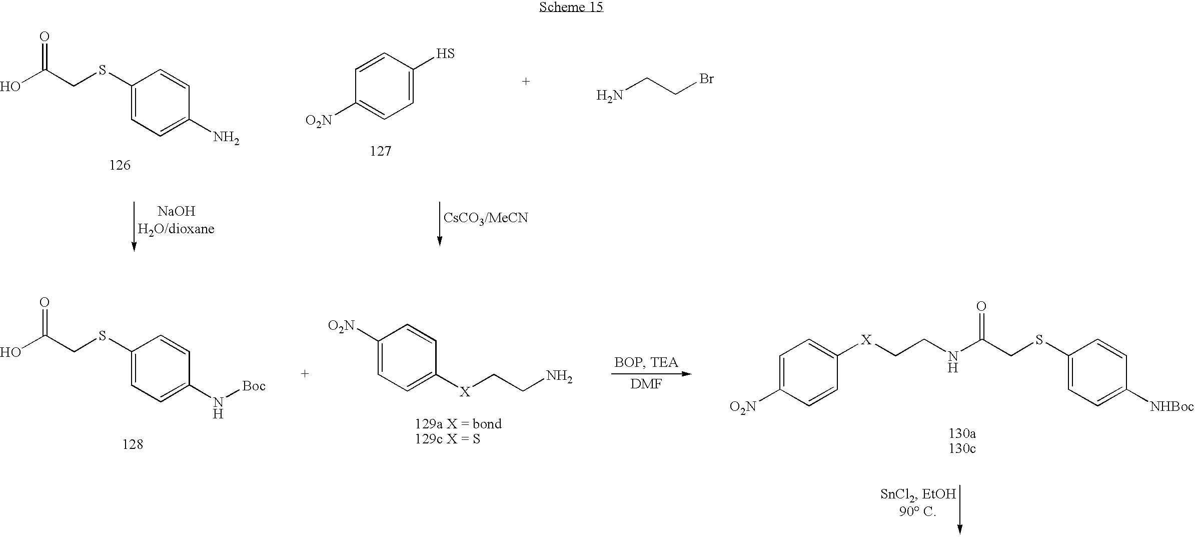

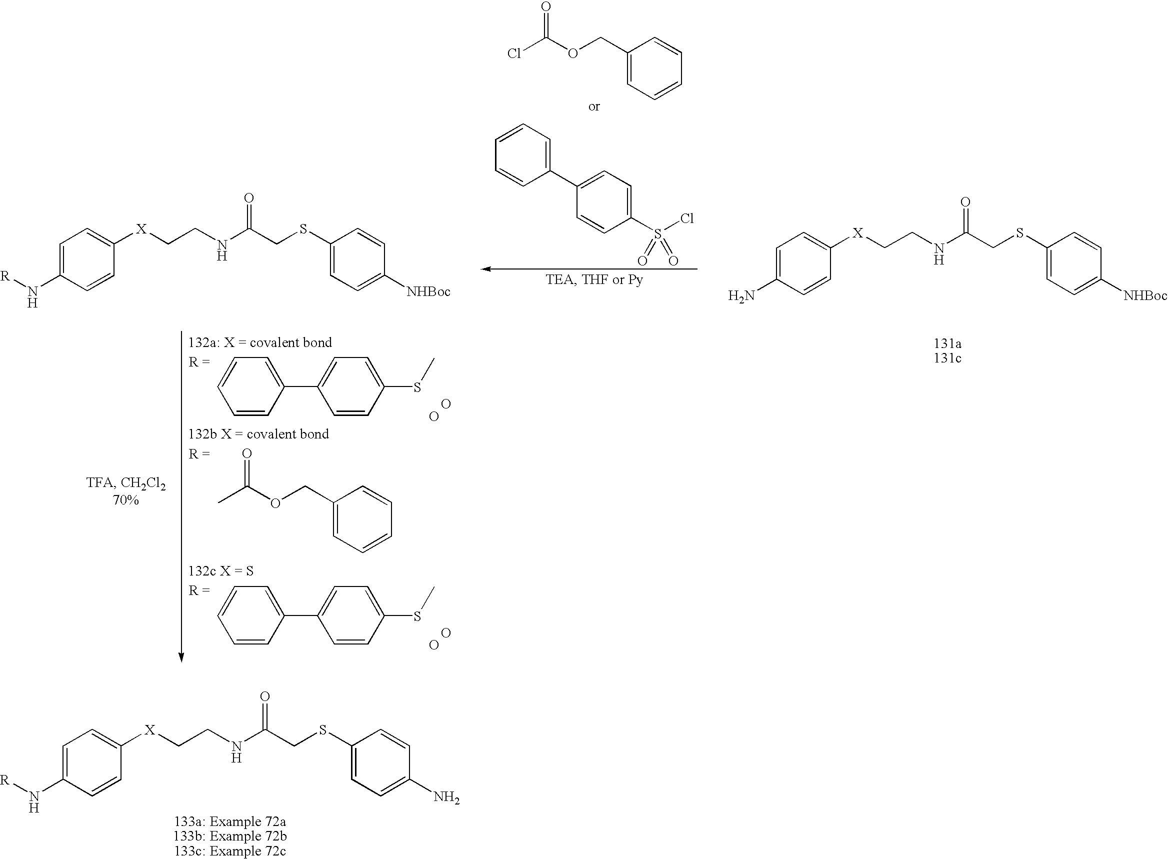

### EXAMPLE 72a

### 2-(4-aminophenylthio)-N-(4-(biphenyl-4-ylsulfonamido)phenethyl)acetamide (133a)

### Step 1: 2-(4-(tert-butoxycarbonylamino)phenylthio)acetic acid (128)

To a stirred solution of 2-(4-aminophenylthio)acetic acid 126 (678 mg, 3.70 mmol) in dioxane/water (2:1, 15 ml) was added di-tert-butyl dicarbonate (848 mg, 3.90 mmol) and NaOH (326 mg, 8.10 mmol). After stirring at room temperature for 4 hours, a precipitate formed, to which ethyl acetate (35 mL) and 3N HCl (8 mL) were added. Following extraction with ethyl acetate, the organic extract was dried (Na₂SO₄), filtered, and evaporated. The residue of 128 (608 mg, 58%) was isolated as a white solid, and used in the subsequent reaction without further purification. LRMS (ESI): (calc) 283.3; (found) 284.2 (MH)⁺.

### Intermediate: 2-(4-nitrophenylthio)ethanamine (129c) (to make 130c)

To a stirred solution of 4-nitrobenzenethiol 127 (500 mg, 3.22 mmol) in acetonitrile (10 mL) at room temperature was added 2-bromoethaneamine hydrobromide (792 mg, 3.87 mmol) and Cs₂CO₃ (5.25 g, 16.1 mmol). The resulting mixture was then heated to 100° C. for 2 hours, cooled, diluted with brine, basified to pH=12 with NaOH, and extracted with ethyl acetate. The organic extract was dried (Na₂SO₄), filtered, and evaporated. The residue was purified by silica gel column chromatography with a gradient of MeOH (0-10%) in EtOAc to afford 129c (475 mg, 74%) as a light orange solid. LRMS (ESI): (calc) 198.2; (found) 199.4 (MH)⁺.

### Step 2: tert-butyl 4-(2-(4-nitrophenethylamino)-2-oxoethylthio)phenylcarbamate (130a)

Following the same procedure as described for compound 1 (scheme 1, example 1) but substituting acid 128 for N-Boc-caproic acid and amine 129a for 3-phenyl aniline to afford 130a (289 mg, 94%) as a white solid. LRMS (ESI): (calc) 431.5; (found) 432.2 (MH)⁺.

### Step 3: tert-butyl 4-(2-(4-aminophenethylamino)-2-oxoethylthio)phenylcarbamate (131a)

To a stirred solution of tert-butyl 4-(2-(4-nitrophenethylamino)-2-oxoethylthio)phenyl-carbamate 130a (289 mg, 0.670 mmol) in ethanol (10 mL) was added tin(II) chloride dihydrate (604 mg, 2.68 mmol) at room temperature. The resulting mixture was then heated to 90° C. for 30 minutes prior to cooling, dilution with brine, and extraction with ethyl acetate. The residue was purified by silica gel column chromatography with a gradient of EtOAc (90-100%) in hexanes to afford 131a (164 mg, 61%) as a light yellow foam. LRMS (ESI): (calc) 401.5; (found) 402.4 (MH)⁺.

### Step 4: tert-butyl 4-(2-(4-(biphenyl-4-ylsulfonamido)phenethylamino)-2-oxoethylthio)phenylcarbamate (132a)

Following the same procedure as described for compound 54 (scheme 2, example 47) but substituting amine 131a for amine 2 and biphenyl-4-sulfonyl chloride for 3-bromo-propionyl chloride to afford 132a (103 mg, 46%) as white solid. LRMS (ESI): (calc.) 617.8; (found) 640.6 (M+Na)⁺.

### Step 5: 2-(4-aminophenylthio)-N-(4-(biphenyl-4-ylsulfonamido)phenethyl)acetamide (133a)

Following the same procedure as described for compound 2 (step 2, scheme 1, example 1) but substituting 132a for 1 to afford 133a (15 mg, 17%) as a light yellow solid. (MeOD-d4) δ (ppm) ¹H: 8.00-7.92 (m,1H), 7.86-7.80 (m,2H), 7.76-7.70 (m,2H), 7.66-7.61 (m,2H), 7.51-7.38 (m,3H), 7.18-7.13 (m,2H), 7.10-7.03 (m,4H), 6.69-6.64 (m,2H), 3.37-3.31 (m,4H), 2.65 (t, J=7.0 Hz, 2H). LRMS (ESI): (calc) 517.7; (found) 518.7 (MH)⁺.

### EXAMPLE 72b AND 72c

Example 72b, c describe the preparation of compound 133b and 133c using the same procedures as described for compound 133a in Example 72a. Characterization data are presented in a Table 8.

TABLE 8

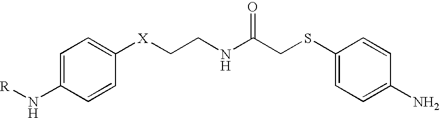

| Ex | Cpd | R | X | Name | Characterization | Scheme |
|---|---|---|---|---|---|---|
| 72b | 133b | 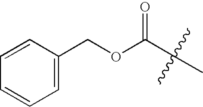 | Covalent bond | benzyl 4-(2-(2-(4- aminophenylthio)- acetamido)ethyl)- phenylcarbamate | (MeOD-d4) δ (ppm) ¹H: 7.46-7.31 (m, 7H), 7.19 (d, J=8.4 Hz, 2H), 7.09 (d, J=8.4 Hz, 2H), 6.66 (d, J=8.4 Hz, 2H), 5.20 (s, 2H), 3.38-3.35 (m, 4H), 2.68 (t, J=7.2 Hz, 2H) LRMS (ESI): (calc) 435.5; (found) 436.7 (MH)⁺. | 15 |
| 72c | 133c | 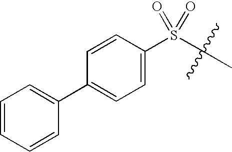 | S | 2-(4- aminophenylthio)- N-(2-(4-(biphenyl- 4- ylsulfonamido)- phenylthio)ethyl)acetamide | (MeOD-d4) δ (ppm) ¹H: 8.18-8.13 (m, 1H), 7.87-7.82 (m, 2H), 7.76-7.71 (m, 2H), 7.65-7.60 (m, 2H), 7.52-7.37 (m, 3H), 7.30-7.24 (m, 2H), 7.23-7.18 (m, 2H), 7.14-7.09 (m, 2H), 6.66-6.00 (m, 2H), [3.44-3.40 (t)], [3.40 (s)], 3.29 (s, 2H), 3.26 (t, J=7.2 | |
| # Hz, 2H), [3.12-3.07 (t)], 2.86 (t, J=6.7 Hz, 2H) LRMS (ESI): (calc) 549.7; (found) 550.7 (MH)⁺. | 15 | | | | | |

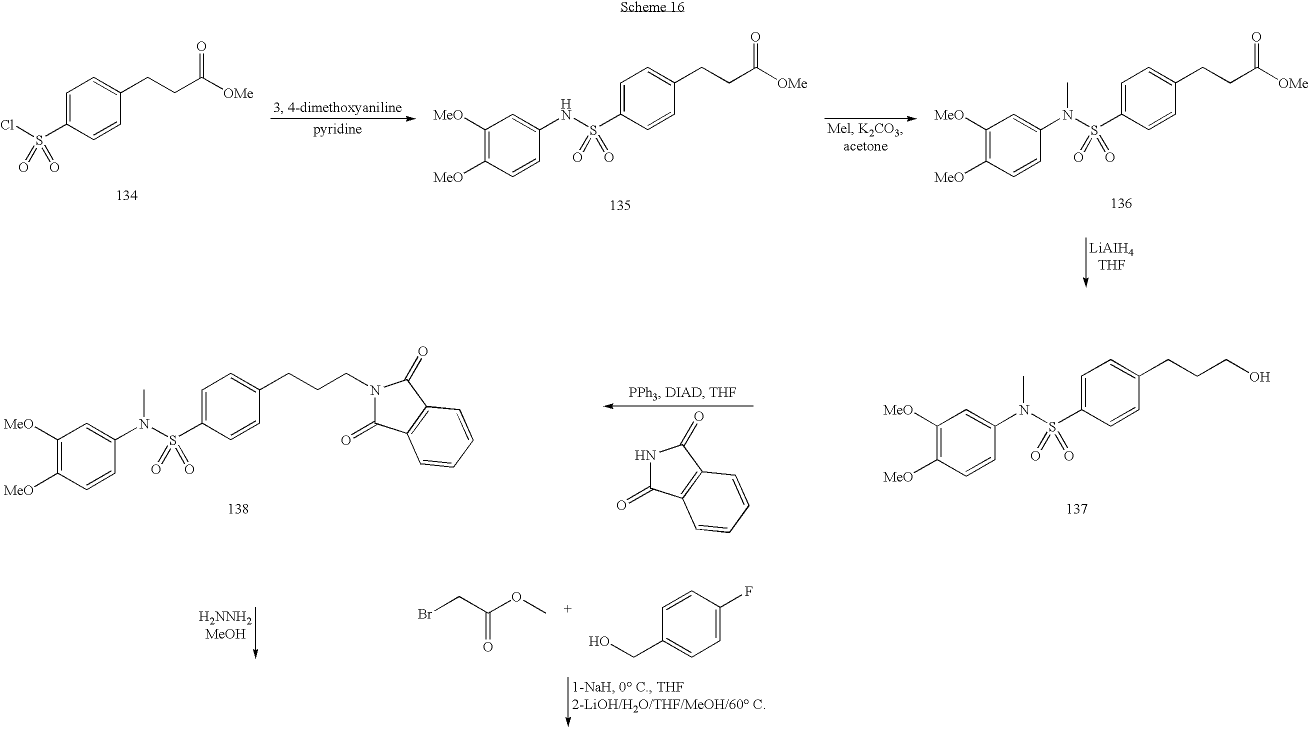

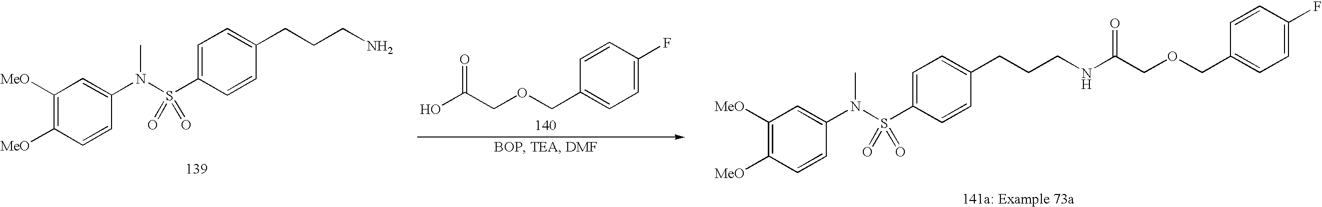

### EXAMPLE 73a

### N-(3-(4-(N-(3,4-dimethoxyphenyl)-N-methylsulfamoyl)phenyl)propyl)-2-(4-fluorobenzyloxy)acetamide (141a)

### Step 1: methyl 3-(4-(N-(3,4-dimethoxyphenyl)sulfamoyl)phenyl)propanoate (135)

Following the same procedure as described for compound 54 (scheme 2, example 47) but substituting amine 3,4-dimetoxyaniline for amine 2, and 134 for 3-bromo-propionyl chloride and using pyridine (some examples DMAP was used) as a solvent and base to afford 135 (1.90 g, 89%) as light purple solid. LRMS (ESI): (calc.) 379.4; (found) 402.1 (M+Na)⁺.

### Step 2: methyl 3-(4-(N-(3,4-dimethoxyphenyl)-N-methylsulfamoyl)phenyl)propanoate (136)

Following the same procedure as described for compound 73 (scheme 5, example 55) but substituting 135 for 72 and using acetone as a solvent to afford 136 (740 mg, 95%) as a light brown foam. LRMS (ESI): (calc.) 393.5, (found) 432.2 (M+K)⁺.

### Step 3: N-(3,4-dimethoxyphenyl)-4-(3-hydroxypropyl)-N-methylbenzenesulfonamide (137)

To a stirred solution of methyl 3-(4-(N-(3,4-dimethoxyphenyl)-N-methylsulfamoyl)phenyl)propanoate 136 (740 mg, 1.88 mmol) in tetrahydrofuran (10 mL) at 0° C. was added lithium aluminum hydride (178 mg, 4.70 mmol). The resulting solution was stirred for 30 minutes prior to quench with water, acidification to pH=1 with HCl, and extraction with ethyl acetate. The organic extract was dried (Na₂SO₄), filtered, and evaporated. The residue comprised of 137 (560 mg, 81%) as a light brown foam was used in the subsequent reaction without further purification. LRMS (ESI): (calc) 365.4; (found) 366.2 (MH)⁺.

### Step 4: N-(3,4-dimethoxyphenyl)-4-(3-(1,3-dioxoisoindolin-2-yl)propyl)-N-methylbenzenesulfonamide (138)

To a stirred solution of N-(3,4-dimethoxyphenyl)-4-(3-hydroxypropyl)-N-methylbenzenesulfonamide 137 (560 mg, 1.53 mmol) in tetrahydrofuran (5 mL) at 0° C. was added phthalimide (306 mg, 2.08 mmol) and triphenylphosphine (532 mg, 2.03 mmol). After stirring for 5 minutes, DIAD (di-isopropylazodicarboxylate) (0.39 mL, 1.99 mmol) was added dropwise, and the reaction warmed to room temperature. After stirring for 1 hour, the solution was diluted with brine, acidified to pH=2 with HCl, and extracted with ethyl acetate. The organic extract was dried (Na₂SO₄), filtered, and evaporated. The residue was purified by silica gel column chromatography using EtOAc (0-100%) in hexanes to afford 138 (546 mg, 72%) as a white foam. LRMS (ESI): (calc) 494.5; (found) 495.2 (MH)⁺.

### Step 5: 4-(3-aminopropyl)-N-(3,4-dimethoxyphenyl)-N-methylbenzenesulfonamide (139)

To a stirred solution of N-(3,4-dimethoxyphenyl)-4-(3-(1,3-dioxoisoindolin-2-yl)propyl)-N-methylbenzenesulfonamide 138 (546 mg, 1.10 mmol) in methanol (4 mL) at room temperature was added hydrazine hydrate (0.11 mL, 2.20 mmol), and the resulting solution was stirred for 16 hours. After filtering the solution through a pad of celite, the filtrate was evaporated to afford 139 (398 mg, 99%) as a light yellow foam. LRMS (ESI): (calc) 364.4; (found) 365.2 (MH)⁺.

### Intermediate: 2-(4-fluorobenzyloxy)acetic acid (140)

Following the same procedure as described for compound 43 (scheme 2, example 37) but substituting (4-fluorophenyl)methanol for (4-(methylthio)phenyl)methanol to afford 140 (13.0 g, 71%) as a white solid. (DMSO-d6) δ (ppm) ¹H: 12.62 (br s,1H), 7.37 (dd, J=8.2, 5.5 Hz, 2H), 7.50 (t, J=8.8 Hz, 2H), 4.49 (s, 2H), 4.04 (s, 2H). LRMS (ESI): (calc.) 184.2; (found) 207.2 (M+Na)⁺.

### Step 6: N-(3-(4-(N-(3,4-dimethoxyphenyl)-N-methylsulfamoyl)phenyl)propyl)-2-(4-fluorobenzyloxy)acetamide (141a)

Following the same procedure as described for compound 1 (scheme 1, example 1) but substituting acid 140 for N-Boc-caproic acid and amine 139 for 3-phenyl aniline to afford 141a (44 mg, 1%) as a light pink oil. (MeOD-d4) δ (ppm) ¹H: 7.51-7.36 (m,6H), 7.14-7.07 (m,2H), 6.85 (d, J=8.6 Hz, 1H), 6.64 (d, J=2.5 Hz, 1H), 6.58 (dd, J=8.6,2.5 Hz, 1H), 4.59 (s,2H), 3.95 (s,2H), 3.82 (s,3H), 3.69 (s,3H), 3.29 (t, J=7.0 Hz, 2H), 3.15 (s,3H), 2.73 (t, J=8.0 Hz, 2H), 1.92-1.83 (m,2H). LRMS (ESI): (calc) 530.6; (found) 531.3 (MH)⁺.

### EXAMPLE 73b

Example 73b describe the preparation of compound 141b using the same procedures as described for compound 141a in Example 73a. Characterization data are presented in a Table 9.

TABLE 9

| Ex | Cpd | structure | Name | Characterization | Scheme |
|---|---|---|---|---|---|
| 73b | 141b | 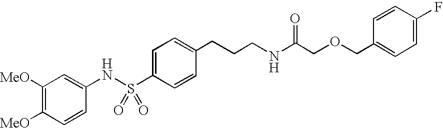 | N-(3-(4-(N-(3,4- dimethoxyphenyl)- sulfamoyl)phenyl)- propyl)-2-(4- fluorobenzyloxy)- acetamide | (MeOD-d4) δ (ppm) ¹H: 7.67-7.62 (m, 2H), 7.46-7.40 (m, 2H), 7.37-7.32 (m, 2H), 7.15-7.07 (m, 2H), 6.80 (d, J=8.6 Hz, 1H), 6.69 (d, J=2.3 Hz, 1H), 6.62-6.56 (m, 1H), 4.58 (s, 2H), 3.93 (s, 2H), 3.77 (s, 3H), 3.72 (s, 3H), 3.26 (t, J=7.0 Hz, 2H), 2.70 (t, | |
| # J=8.0 Hz, 2H), 1.90-1.80 (m, 2H) LRMS (ESI): (calc) 516.6; (found) 517.2 (MH)⁺. | 16 (steps 1, 3-6) | | | | |
| 73c | 141c | 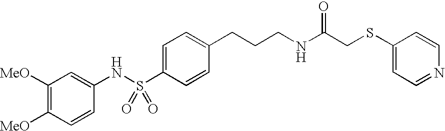 | N-(3-(4-(N-(3,4- dimethoxyphenyl)- sulfamoyl)phenyl)- propyl)-2-(pyridin- 4-ylthio)acetamide | (MeOD-d4) δ (ppm) ¹H: 8.34 (dd, J=4.7, 1.6 Hz, 2H), 7.64-7.59 (m, 2H), 7.36 (dd, J=4.7, 1.6 Hz, 2H), 7.28-7.23 (m, 2H), 6.78 (d, J=8.6 Hz, 1H), 6.68 (d, J=2.3 Hz, 1H), 6.58 (dd, J=8.6, 2.5 Hz, 1H), 3.81 (s, 2H), 3.76 (s, 3H), 3.71 (s, 3H), 3.22 (t, J=6.8 | |
| # Hz, 2H), 2.62 (t, J=8.0 Hz, 2H), 1.83-1.74 (m, 2H) LRMS (ESI): (calc) 501.6; (found) 502.6 (MH)⁺. 16 (steps 1, 3-6) | | | | | |

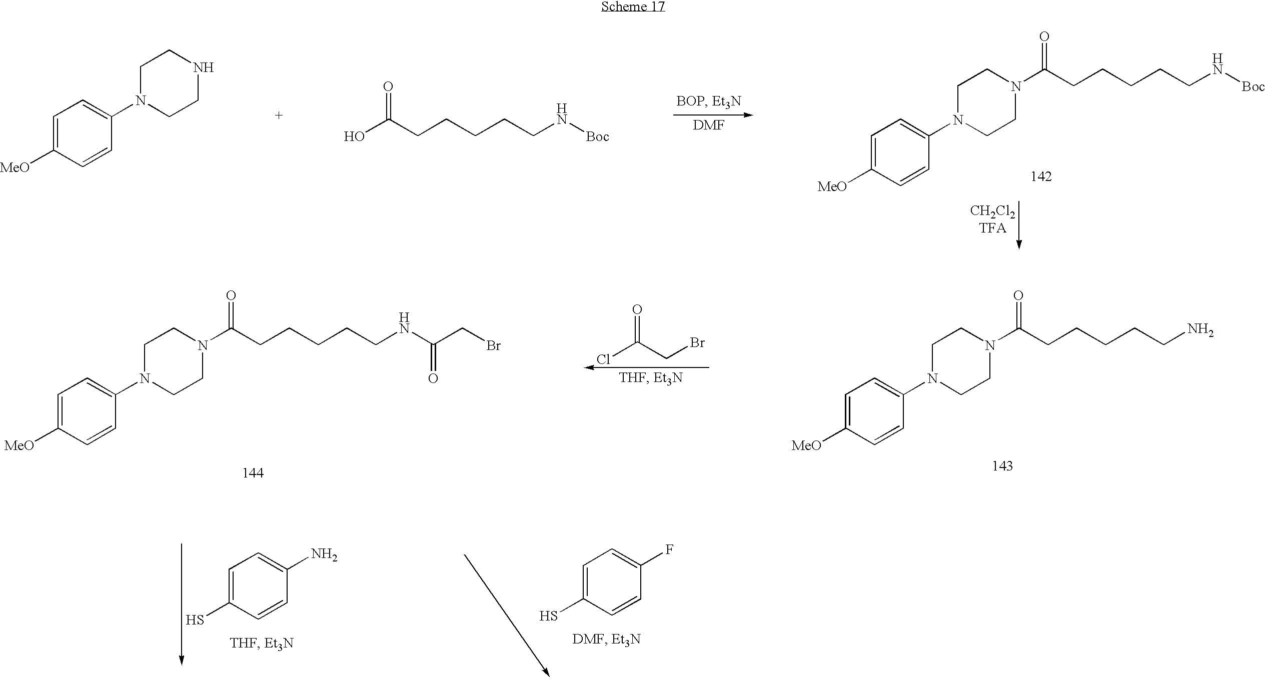

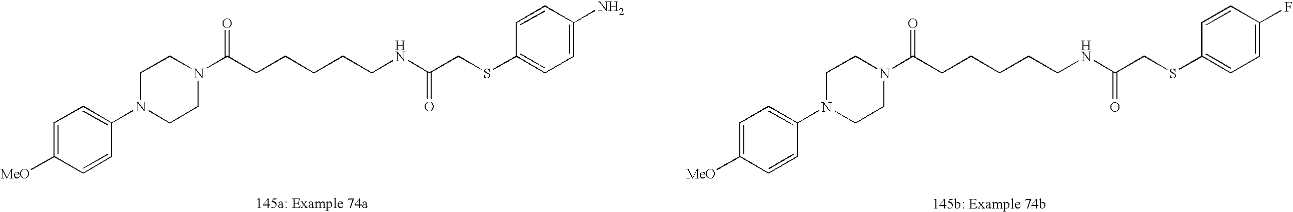

### EXAMPLE 74a

### 2-(4-aminophenylthio)-N-(6-(4-(4-methoxyphenyl)piperazin-1-yl)-6-oxohexyl)acetamide (145a)

### Step 1: tert-butyl 6-(4-(4-methoxyphenyl)piperazin-1-yl)-6-oxohexylcarbamate (142)

Following the same procedure as described for compound 1 (scheme 1, example 1) but substituting 1-(4-methoxyphenyl)piperazine for 3-phenyl aniline to afford 142 (5.22 g, 99%) as a yellow oil. LRMS (ESI): (calc) 405.5; (found) 406.2 (MH)⁺.

### Step 2: 6-amino-1-(4-(4-methoxyphenyl)piperazin-1-yl)hexan-1-one (143)

Following the same procedure as described for compound 2 (step 2, scheme 1, example 1) but substituting 142 for 1 to afford 143 (2.08 g, 53%) as an orange oil. LRMS (ESI): (calc) 305.4; (found) 306.4 (MH)⁺.

### Step 3: 2-bromo-N-(6-(4-(4-methoxyphenyl)piperazin-1-yl)-6-oxohexyl)acetamide (144)

Following the same procedure as described for compound 3b (step 3, scheme 1, example 1) but substituting 143 for 2 to afford 144 (934 mg, 67%) as a yellow oil. LRMS (ESI): (calc) 426.4; (found) 427.5 (MH)⁺.

### Step 4: 2-(4-aminophenylthio)-N-(6-(4-(4-methoxyphenyl)piperazin-1-yl)-6-oxohexyl)acetamide (145a)

Following the same procedure as described for compound 4 (step 4, scheme 1, example 1) but substituting 144 for 3b and 4-aminobenzenethiol for methyl 2-mercaptoacetate to afford 145a (165 mg, 75%) as a yellow oil. (MeOD-d4) δ (ppm) ¹H: 7.23 (d, J=8.6 Hz, 2H), 6.97 (d, J=9.2 Hz, 2H), 6.86 (d, J=9.2 Hz, 2H), 6.65 (d, J=8.6 Hz, 2H), 3.77 (s,3H), 3.76-3.72 (m,2H), 3.71-3.67 (m,2H), 3.38 (m,2H), 3.17 (t, J=6.8 Hz, 2H), 3.07 (t, J=5.1 Hz, 2H), 3.02 (t, J=5.1 Hz, 2H), 2.40 (t, J=7.8 Hz, 2H), 1.65-1.58 (m,2H), 1.51-1.42 (m,2H), 1.34-1.26 (m,2H). LRMS (ESI): (calc) 470.2; (found) 471.6 (MH)⁺.

### EXAMPLE 74b

### 2-(4-fluorophenylthio)-N-(6-(4-(4-methoxyphenyl)piperazin-1-yl)-6-oxohexyl)acetamide (145b)

### Step 1-4: 2-(4-fluorophenylthio)-N-(6-(4-(4-methoxyphenyl)piperazin-1-yl)-6-oxohexyl)acetamide (145b)

Following the same procedure as described for compound 145a (step 1-4, scheme 17, example 74a) but substituting 4-fluorobenzenethiol for 4-aminobenzenethiol to afford 145b (445 mg, 85%) as a white solid. (MeOD-d4) δ (ppm) ¹H: 7.47 (t, J=6.8 Hz, 2H), 7.09 (t, J=8.6 Hz, 2H), 6.98 (d,-J=9.0 Hz, 2H), 6.86 (d, J=8.8 Hz, 2H), 3.77 (s,3H), 3.76-3.73 (m,2H), 3.72-3.68 (m,2H), 3.57 (s,2H), 3.18 (t, J=6.7 Hz, 2H), 3.09-3.07 (m,2H), 3.04-3.02 (m,2H), 2.42 (t, J=7.4 Hz, 2H), 1.63-1.56 (m,2H), 1.51-1,44 (m,2H), 1.33-1.26 (m,2H). LRMS (ESI): (calc) 473.6; (found) 474.5 (MH)⁺.

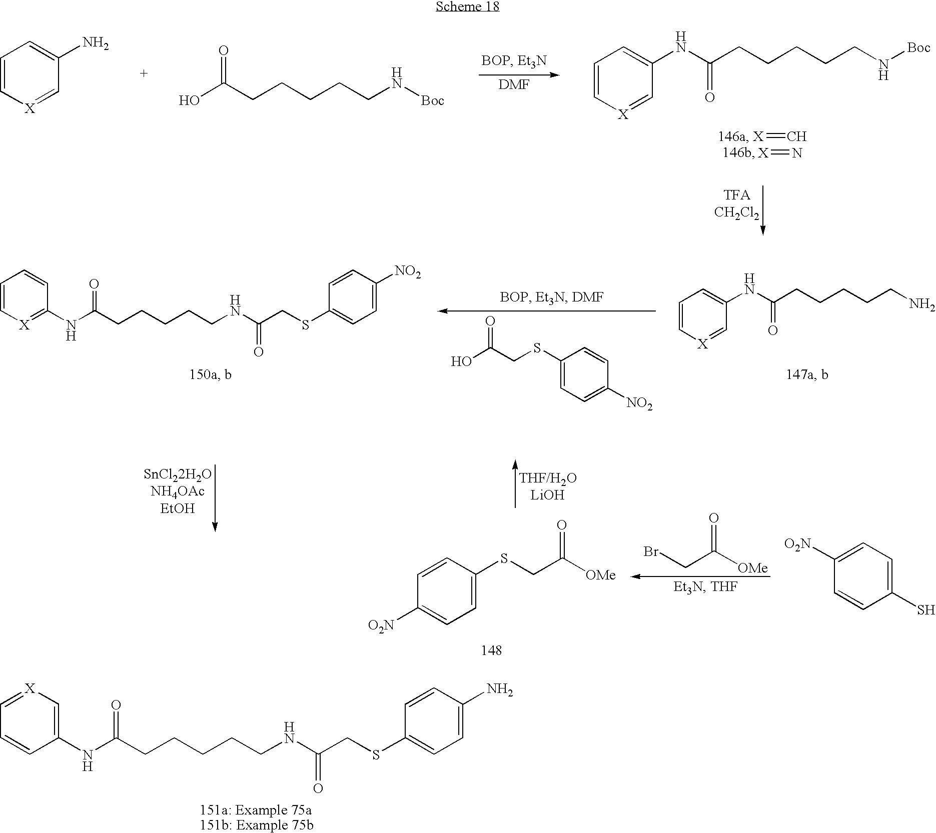

### EXAMPLE 75a

### 6-(2-(4-aminophenylthio)acetamido)-N-phenylhexanamid (151a)

### Step 1: tert-butyl 6-oxo-6-(phenylamino)hexylcarbamate (146a)

Following the same procedure as described for compound 1 (scheme 1, example 1) but substituting aniline for 3-phenyl aniline to afford 146a (2.90 g, 32%) as a light yellow solid. LRMS (ESI): (calc) 306.2; (found) 307.4 (MH)⁺.

### Step 2: 6-amino-N-phenylhexanamide (147a)

Following the same procedure as described for compound 2 (step 2, scheme 1, example 1) but substituting 146a for 1 to afford 147a (631 mg, 69%) as a light yellow oil. LRMS (ESI): (calc) 206.3; (found) 207.2 (MH)⁺.

### Intermediate: 2-(4-nitrophenylthio)acetic acid (149)

### Step A: methyl 2-(4-nitrophenylthio)acetate (148)

Following the same procedure as described for compound 4 (step 4, scheme 1, example 1) but substituting methyl 2-bromoacetate for 3b and 4-nitrobenzenethiol for methyl 2-mercaptoacetate to afford 148 (1.33 g, 90%) as an orange solid. LRMS (ESI): (calc) 227.2; (found) 228.2 (MH)⁺.

### Step B: 2-(4-nitrophenylthio)acetic acid (149)

Following the same procedure as described for compound 5 (step 5, scheme 1, example 1) but substituting 148 for 4 to afford 149 (1.12 g, 94%) as a yellow solid. LRMS (ESI): (calc) 213.0; (found) 211.9 (M−H⁺).

### Step 3: 6-(2-(4-nitrophenylthio)acetamido)-N-phenylhexanamide (150a)

Following the same procedure as described for compound 1 (scheme 1, example 1) but substituting acid 149 for N-Boc-caproic acid and amine 147a for 3-phenyl aniline to afford 150a (361 mg, 57%) as a light yellow solid. LRMS (ESI): (calc) 401.5; (found) 402.7 (MH)⁺.

### Step 4: 6-(2-(4-aminophenylthio)acetamido)-N-phenylhexanamide (151a)

To a stirred solution of 6-(2-(4-nitrophenylthio)acetamido)-N-phenylhexanamide 150a (204 mg, 0.51 mmol) in ethanol (10 mL) was added tin(II) chloride dihydrate (460 mg, 204 mmol) and NH₄OAc (393 mg, 5.10 mmol) at room temperature. After heating to 100° C. for 20 minutes, the solution was cooled, diluted with water, basified to pH=9 with NaOH, and extracted with ethyl acetate. The combined organic extracts were dried (Na₂SO₄), filtered, and evaporated. The residue was purified by silica gel column chromatography using EtOAc (0-100%) in hexanes to afford 151a (155 mg, 82%) as a colorless oil. (MeOD-d4) δ (ppm) ¹H: 8.57-8.49 (m,1H), 7.57-7.50 (m,2H), 7.33-7.24 (m,2H), 7.23-7.17 (m,2H), 7.11-7.02 (m,1H), 6.68-6.60 (m,2H), 3.39-3.34 (m,2H), 3.18-3.10 (m,2H), 2.40-2.31 (m,2H), 1.74-1.62 (m,2H), 1.51-1.41 (m,2H), 1.35-1.23(m,2H). LRMS (ESI): (calc) 371.5; (found) 394.0 (M+Na)⁺.

### EXAMPLE 75b

### 6-(2-(4-aminophenylthio)acetamido)-N-(pyridin-3-yl)hexanamide (151b)

### Step 1-4: 6-(2-(4-aminophenylthio)acetamido)-N-(pyridin-3-yl)hexanamide (151b)

Following the same procedure as described for compound 151a (step 1-4, scheme 18, example 75a) but substituting pyridin-3-amine for aniline to afford 151b (252 mg, 79%) as an orange oil. (MeOD-d4) δ (ppm) ¹H: 8.77-8.73 (m,1H), 8.27-8.24 (m,1H), 8.15-8.11 (m,1H), 8.00-7.95 (m,1H), 7.43-7.38 (m,1H), 7.24 (s,1H), 7.21 (s,1H), 6.67 (s,1H), 6.65 (s,1H), 3.39-3.38 (m,2H), 3.21-3.14 (m,2H), 2.42 (t, J=7.4 Hz, 2H), 1.75-1.66 (m,2H), 1.53-1.43 (m,2H), 1.36-1.27 (m,2H). LRMS (ESI): (calc) 372.5; (found) 373.2 (MH)⁺.

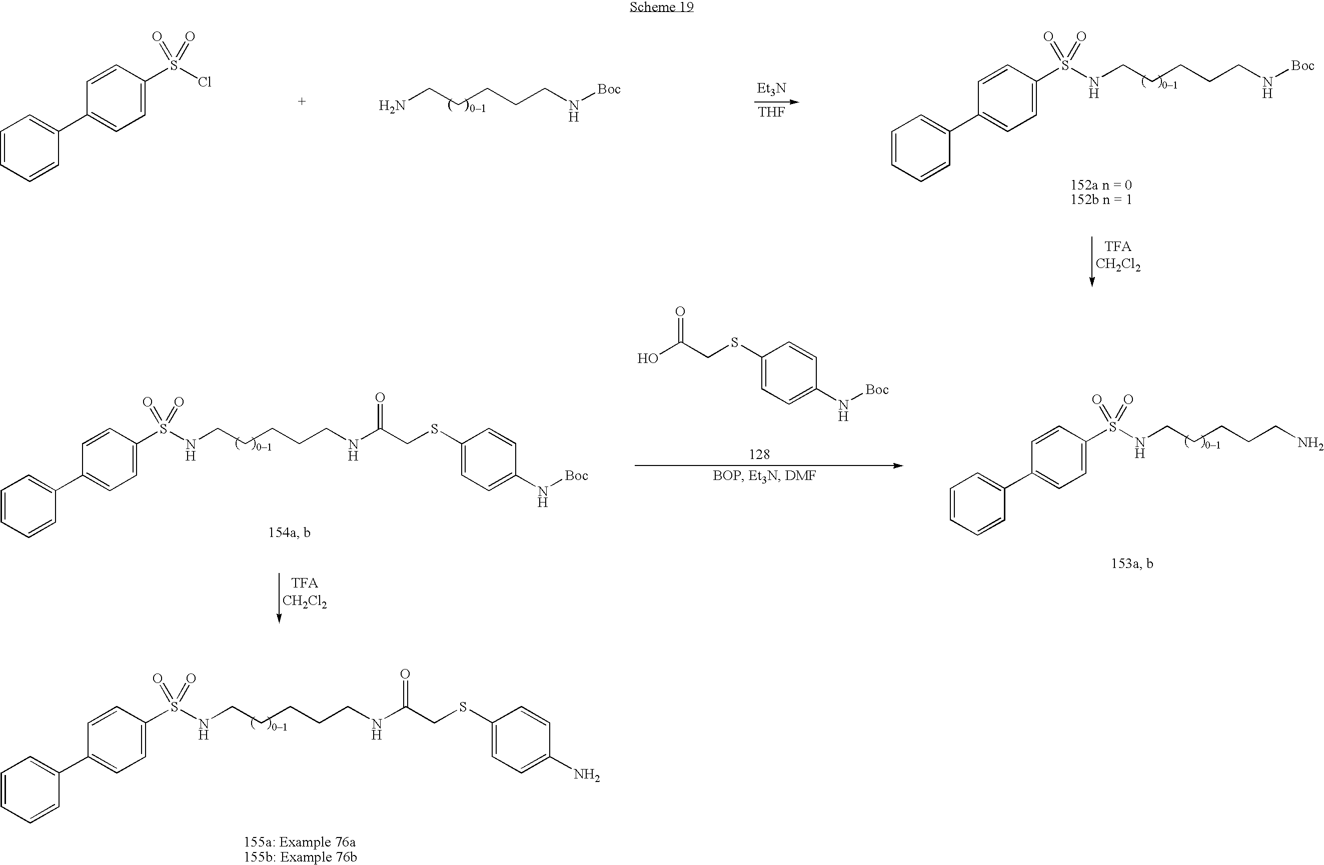

### EXAMPLE 76a

### 2-(4-aminophenylthio)-N-(4-(biphenyl-4-ylsulfonamido)butyl)acetamide (155a)

### Step 1: tert-butyl 4-(biphenyl-4-ylsulfonamido)butylcarbamate (152a)

Following the same procedure as described for compound 54 (scheme 2, example 47) but substituting tert-butyl 4-aminobutylcarbamate for amine 2 and biphenyl-4-sulfonyl chloride for 3-bromo-propionyl chloride to afford 152a (534 mg, quantitative) as a yellow solid. LRMS (ESI): (calc.) 404.2; (found) 405.3 (MH)⁺.

### Step 2: N-(4-aminobutyl)biphenyl-4-sulfonamide (153a)

Following the same procedure as described for compound 2 (step 2, scheme 1, example 1) but substituting 152a for 1 to afford 153a (374 mg, 87%) as a yellow solid. LRMS (ESI): (calc) 304.1; (found) 305.0 (MH)⁺.

### Step 3: tert-butyl 4-(2-(4-(biphenyl-4-ylsulfonamido)butylamino)-2-oxoethylthio)phenylcarbamate (154a)

Following the same procedure as described for compound 1 (scheme 1, example 1) but substituting acid 128 for N-Boc-caproic acid and amine 153a for 3-phenyl aniline to afford 154a (284 mg, 70%) as a white solid. LRMS (ESI): (calc) 569.2; (found) 570.2 (MH)⁺.

### Step 4: 2-(4-aminophenylthio)-N-(4-(biphenyl-4-ylsulfonamido)butyl)acetamide (155a)

Following the same procedure as described for compound 2 (step 2, scheme 1, example 1) but substituting 154a for 1 to afford 155a (235 mg, 74%) as a white solid. (DMSO-d6) δ (ppm) ¹H: 7.95-7.87 (m,5H), 7.80-7.76 (m,2H), 7.68 (t, J=8.0 Hz, 1H), 7.56-7.53 (m,2H), 7.50-7.46 (m,1H), 7.12 (d, J=8.4 Hz, 2H), 6.52 (d, J=8.4 Hz, 2H), 5.69 (s,2H), 3.32 (s,2H), 3.02-2.99 (m,2H), 2.81-2.74 (m,2H), 1.40-1.34 (m,4H). LRMS (ESI): (calc) 469.6; (found) 470.0 (MH)⁺.

### EXAMPLE 76b

### 2-(4-aminophenylthio)-N-(5-(biphenyl-4-ylsulfonamido)pentyl)acetamide (155b)

### Step 1-4: 2-(4-aminophenylthio)-N-(5-(biphenyl-4-ylsulfonamido)pentyl)acetamide (155b)

Following the same procedure as described for compound 155a (step 14, scheme 19, example 76a) but substituting tert-butyl 5-aminopentylcarbamate for tert-butyl 4-aminobutylcarbamate to afford 155b (42 mg, 73%) as a white solid. (MeOD-d4) δ (ppm) ¹H: 7.97-7.93 (m,2H), 7.87-7.84 (m,2H), 7.74-7.70 (m,2H), 7.54-7.49 (m,2H), 7.47-7.42 (m,1H), 7.24-7.20 (m,2H), 6.68-6.63 (m,2H), 3.36 (s,2H), 3.09 (t, J=6.9 Hz, 2H), 2.88 (t, J=7.0 Hz, 2H), 1.49-1.41 (m,2H), 1.40-1.31 (m,2H), 1.25-1.15 (m,2H). LRMS (ESI): (calc) 483.6; (found) 484.7 (MH)⁺.

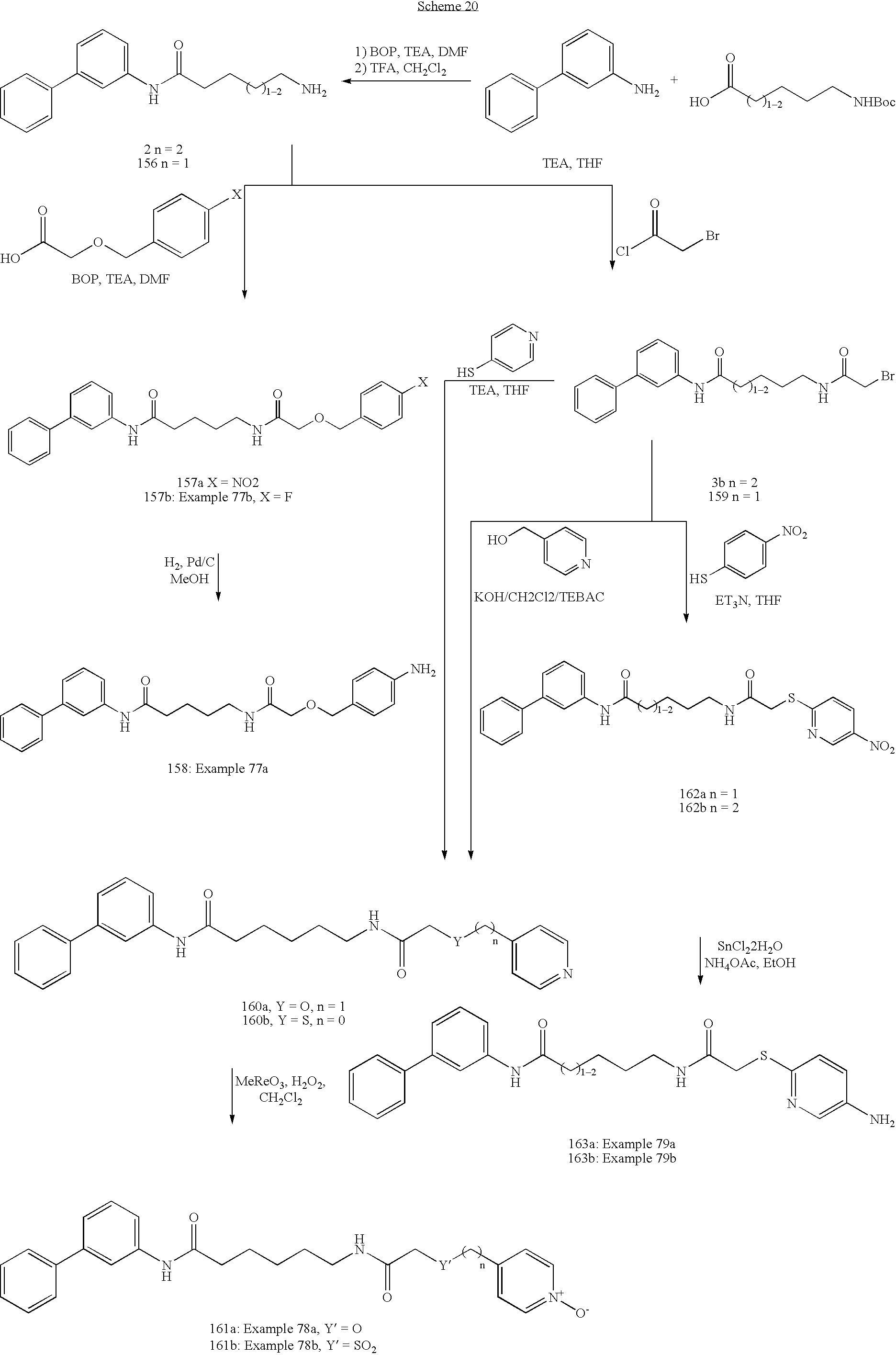

### EXAMPLE 77a

### 5-(2-(4-aminobenzyloxy)acetamido)-N-(biphenyl-3-yl)pentanamide (158)

### Step 1: 5-amino-N-(biphenyl-3-yl)pentanamide (156)

Following the same procedure as described for compound 2 (step 1 and 2, scheme 1, example 1) but substituting 5-(tert-butoxycarbonylamino)pentanoic acid for 6-(tert-butoxycarbonylamino)hexanoic acid to afford 156 (4.10 g, 95%) as light orange oil. LRMS (ESI): (calc) 368.5; (found) 369.2 (MH)⁺.

### Intermediate: 2-(4-nitrobenzyloxy)acetic acid

Following the same procedure as described for compound 43 (step 1 and 2, scheme 2, example 37) but substituting (4-nitrophenyl)methanol for (4-(methylthio)phenyl)methanol to afford 2-(4-nitrobenzyloxy)acetic acid (1.56 g, 77%) as light orange solid. LRMS (ESI): (calc) 211.2; (found) 210.0 (M−H⁺).

### Step 2: N-(biphenyl-3-yl)-5-(2-(4-nitrobenzyloxy)acetamido)pentanamide (157a)

Following the same procedure as described for compound 1 (step 1, scheme 1, example 1) but substituting 2-(4-nitrobenzyloxy)acetic acid for N-Boc-caproic acid and amine 156 for 3-phenyl aniline to afford 157a (253 mg, 44%) as an orange oil. LRMS (ESI): (calc) 461.5; (found) 462.3 (MH)⁺.

### Step 3: 5-(2-(4-aminobenzyloxy)acetamido)-N-(biphenyl-3-yl)pentanamide (158)

Following the same procedure as described for compound 74 (step 3, scheme 5, example 55) but substituting 157a for 73 to afford 158 (7 mg, 3%) as a white foam. (MeOD-d4) δ (ppm) ¹H: 7.87 (t, J=1.6 Hz, 1H), 7.64-7.58 (m,2H), 7.57-7.52 (m,1H), 7.47-7.31 (m,5H), 7.12 (d, J=8.4 Hz, 2H), 6.69 (d, J=8.4 Hz, 2H), 4.45 (s,2H), 3.89 (s,2H), 3.29 (t, J=6.8 Hz, 2H), 2.46 (t, J=5.1 Hz, 2H), 1.80-1.70 (m,2H), 1.67-1.56 (m,2H). LRMS (ESI): (calc) 431.5; (found) 432.2 (MH)⁺.

### EXAMPLE 77b

### N-(biphenyl-3-yl)-5-(2-(4-fluorobenzyloxy)acetamido)pentanamide (157b)

### Step 1-2: N-(biphenyl-3-yl)-5-(2-(4-fluorobenzyloxy)acetamido)pentanamide (157b)

Following the same procedure as described for compound 157a (scheme 20, example 77a) but substituting 2-(4-fluorobenzyloxy)acetic acid (140) for 2-(4-nitrobenzyloxy)acetic acid to afford 157b (75 mg, 86%) as light yellow solid. (MeOD-d4) δ (ppm) ¹H, 7.89 (t, J=1.8 Hz, 1H), 7.63-7.58 (m,2H), 7.55 (dt, J=7.6,1.6 Hz, 1H), 7.46-7.31 (m,7H), 7.10-7.04 (m,2H), 4.56 (s,2H), 3.95 (s,2H), 3.30 (t, J=6.8 Hz, 2H), 2.44 (t, J=7.0 Hz, 2H), 1.80-1.70 (m,2H), 1.67-1.57 (m,2H). LRMS (ESI): (calc) 434.5; (found) 435.2 (MH)⁺.

### EXAMPLE 78a

### 4-((2-(6-(biphenyl-3-ylamino)-6-oxohexylamino)-2-oxoethoxy)methyl)pyridine 1-oxide (161a)

### Step 1: N-(biphenyl-3-yl)-6-(2-(pyridin-4-ylmethoxy)acetamido)hexanamide (160a)

Following the same procedure as described for compound 7a (step 4, scheme 1, example 4) but substituting 2 pyridin-4-ylmethanol for tert-butyl-2-hydroxyethylcarbamate to afford 160a (83 mg, 52%) as a yellow oil. LRMS (ESI): (calc) 431.7; (found) 432.2 (MH)⁺.

### Step 2: 4-((2-(6-(biphenyl-3-ylamino)-6-oxohexylamino)-2-oxoethoxy)methyl)pyridine 1-oxide (161a)

To a stirred solution of 160a (83 mg, 0.192 mmol) in dichloromethane (3.5 mL) was added methyl trioxorhenium (3 mg, 0.013 mmol) and hydrogen peroxide (35% by wt in water, 0.02 mL, 0.231 mmol). The resulting solution was stirred for 2 hours prior to evaporation of solvents, and direct purification of the residue by silica gel column chromatography with a gradient of MeOH (040%) in EtOAc to afford 161a (73 mg, 85%) as a light yellow oil. (MeOD-d4) δ (ppm) ¹H: 8.32-8.23 (m,3H), 7.87 (t, J=1.8 Hz, 1H), 7.61-7.56 (m,2H), 7.55-7.50 (m,3H), 7.45-7.31 (m,4H), 4.68 (s,1H), 4.59 (s,2H), 4.03 (s,2H), 3.30 (t, J=6.8 Hz, 2H), 2.44 (t, J=7.2 Hz, 2H), 1.82-1.72 (m,2H), 1.66-1.56 (m,2H), 1.50-1.40 (m,2H). LRMS (ESI): (calc) 447.5; (found) 448.5 (MH)⁺.

### EXAMPLE 78b

### 4-(2-(6-(biphenyl-3-ylamino)-6-oxohexylamino)-2-oxoethylsulfonyl)pyridine 1-oxide (161b)

### Step 1: N-(biphenyl-3-yl)-6-(2-(pyridin-4-ylthio)acetamido)hexanamide (160b)

Following the same procedure as described for compound 4 (step 4, scheme 1, example 1) but substituting pyridine-4-thiol for 4-aminobenzenethiol to afford 160b (75 mg, 66%) as a light yellow solid. (MeOD-d4) δ (ppm) ¹H: 8.34-8.30 (m,2H), 7.87 (t, J=1.6 Hz, 1H), 7.64-7.58 (m,2H), 7.57-7.53 (m,1H), 7.47-7.30 (m,7H), 3.80 (s,2H), 3.25 (t, J=6.8 Hz, 2H), 2.40 (t, J=7.4 Hz, 2H), 1.78-1.68 (m,2H), 1.62-1.52 (m,2H), 1.44-1.35 (m,2H). LRMS (ESI): (calc) 433.6; (found) 434.4 (MH)⁺.

### Step 2: 4-(2-(6-(biphenyl-3-ylamino)-6-oxohexylamino)-2-oxoethylsulfonyl)pyridine 1-oxide (161b)

Following the same procedure as described for compound 161a (step 2, scheme 20, example 78a) but substituting 160b for 160a to afford 161b (9 mg, 6%) as light yellow solid. (MeOD-d4) δ (ppm) ¹H: 8.49-8.45 (m,2H), 7.95-7.92 (m,2H), 7.89-7.87 (m,1H), 7.64-7.60 (m,2H), 7.58-7.54 (m,1H), 7.48-7.34 (m,5H), 3.38 (s,4H), 3.22 (t, J=6.9 Hz, 2H), 2.44 (t, J=7.4 Hz, 2H), 1.80-1.72 (m,2H), 1.61-1.52 (m,2H), 1.48-1.40 (m,2H). LRMS (ESI): (calc) 481.6; (found) 482.5 (MH)⁺.

### EXAMPLE 79a

### 5-(2-(5-aminopyridin-2-ylthio)acetamido)-N-(biphenyl-3-yl)pentanamide (163a)

### Step 1: N-(biphenyl-3-yl)-5-(2-bromoacetamido)pentanamide (159)

Following the same procedure as described for compound 3b (step 3, scheme 1, example 1) but substituting 156 for 2 to afford 159 (683 mg, 34%) as a light orange solid. LRMS (ESI): (calc) 389.4; (found) 390.1 (MH)⁺.

### Step 2: N-(biphenyl-3-yl)-5-(2-(5-nitropyridin-2-ylthio)acetamido)pentanamide (162a)

Following the same procedure as described for compound 4 (step 4, scheme 1, example 1) but substituting 159 for 3b and 5-nitropyridine-2-thiol for methyl thioglycolate to afford 162a (218 mg, 90%) as an orange oil. LRMS (ESI): (calc) 464.2; (found) 465.2 (MH)⁺.

### Step 3: 5-(2-(5-aminopyridin-2-ylthio)acetamido)-N-(biphenyl-3-yl)pentanamide (163a)

Following the same procedure as described for compound 151a (step 4, scheme 18, example 75a) but substituting 162a for 150a to afford 163a (195 mg, 84%) as a white solid. (MeOD-d4) δ (ppm) ¹H: 7.96-7.94 (m,1H), 7.88-7.87 (m,1H), 7.65-7.61 (m,2H), 7.58-7.54 (m,1H), 7.49-7.34 (m,5H), 7.16-7.12 (m,1H), 7.01-6.96 (m,1H), 3.69 (s,2H), 3.25 (t, J=8.0 Hz, 2H), 2.40 (t, J=8.2 Hz, 2H), 1.73-1.64 (m,2H), 1.60-1.52 (m,2H). LRMS (ESI): (calc) 434.6; (found) 435.1 (MH)⁺.

### EXAMPLE 79b

### 6-(2-(5-aminopyridin-2-ylthio)acetamido)-N-(biphenyl-3-yl)hexanamide (163b)

### Step 1: 6-(2-(5-aminopyridin-2-ylthio)acetamido)-N-(biphenyl-3-yl)hexanamide (163b)

Following the same procedure as described for compound 163a (scheme 20, example 79a) but substituting 3b for 159 in Step 2 to afford 162b, which was then immediately reacted according to the same procedure as described for compound 151a (step 4, scheme 18, example 75a) but substituting the crude material for 150a to afford 163b (19 mg, 20%) as a yellow oily solid. (MeOD-d4) δ (ppm) ¹H: 7.94 (dd, J=2.7,0.8 Hz, 1H), 7.88-7.85 (m,1H), 7.63-7.58 (m,2H), 7.56-7.52 (m,1H), 7.46-7.32 (m,5H), 7.12 (dd, J=8.4,0.8 Hz, 1H), 7.00 (dd, J=5.7,2.7 Hz, 1H), 3.66 (s,2H), 3.21 (t, J=6.8 Hz, 2H), 2.38 (t, J=7.8 Hz, 2H), 1.76-1.65 (m,2H), 1.56-1.46 (m,2H), 1.39-1.29 (m,2H). LRMS (ESI): (calc) 448.6; (found) 449.3 (MH)⁺.

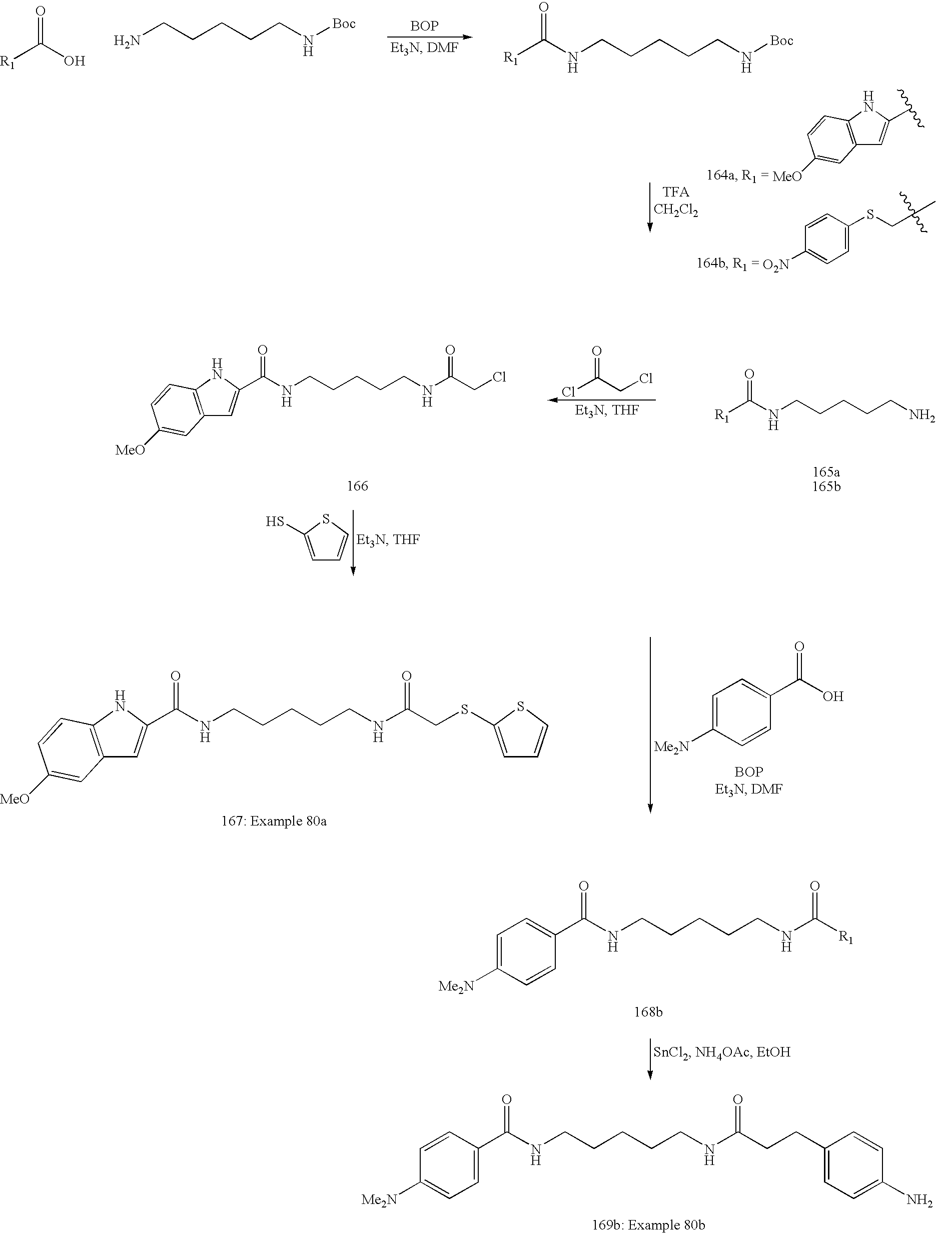

### EXAMPLE 80a

### 5-methoxy-N-(5-(2-(thiophen-2-ylthio)acetamido)pentyl)-1H-indole-2-carboxamide (167)

### Step 1: tert-butyl 5-(5-methoxy-1H-indole-2-carboxamido)pentylcarbamate (164a)

Following the same procedure as described for compound 1 (scheme 1, example 1) but substituting 5-methoxy-1H-indole-2-carboxylic acid for N-Boc-caproic acid and amine tert-butyl 5-aminopentylcarbamate for 3-phenyl aniline to afford 164a (784 mg, 99%) as a yellow oil. LRMS (ESI): (calc) 375.2; (found) 376.2 (MH)⁺.

### Step 2: N-(5-aminopentyl)-5-methoxy-1H-indole-2-carboxamide (165a)

Following the same procedure as described for compound 2 (step 2, scheme 1, example 1) but substituting 164a for 1 to afford 165a (402 mg, 52%) as a yellow oil. LRMS (ESI): (calc) 275.2; (found) 276.3 (MH)⁺.

### Step 3: N-(5-(2-chloroacetamido)pentyl)-5-methoxy-1H-indole-2-carboxamide (166)

Following the same procedure as described for compound 3a (step 3, scheme 1, example 1) but substituting 165a for 2 to afford 166 (436 mg, 85%) as a yellow solid. LRMS (ESI): (calc) 351.8; (found) 352.5 (MH)⁺.

### Step 4: 5-methoxy-N-(5-(2-(thiophen-2-ylthio)acetamido)pentyl)-1H-indole-2-carboxamide (167)

Following the same procedure as described for compound 4 (step 4, scheme 1, example 1) but substituting 166 for 3b and thiophene-2-thiol for methyl 2-mercaptoacetate to afford 167 (156 mg, 98%) as a light yellow solid. (DMSO-d6) δ (ppm) ¹H, 11.42 (s,1H), 8.43 (t, J=5.9 HZ, 1H), 8.04 (t, J=5.5 Hz, 1H), 7.66-7.64 (m,1H), 7.34 (d, J=8.8 Hz, 1H), 7.22-7.20 (m,1H), 7.11-7.03 (m,3H), 6.87-6.83 (m,1H), 3.79 (s,3H), 3.50 (s,2H), 3.32-3.25 (m,2H), 3.08 (q, J=5.9,2.7 Hz, 2H), 1.60-1.50 (m,2H), 1.48-1.39 (m,2H), 1.34-1.25 (m,2H). LRMS (ESI): (calc) 431.6; (found) 432.4 (MH)⁺.

### EXAMPLE 80b

### N-(5-(2-(4-aminophenylthio)acetamido)pentyl)-4-(dimethylamino)benzamide (169b)

### Step 1: tert-butyl 5-(2-(4-nitrophenylthio)acetamido)pentylcarbamate (164b)

Following the same procedure as described for compound 1 (scheme 1, example 1) but substituting 2-(4-nitrophenylthio)acetic acid for N-Boc-caproic acid and amine tert-butyl 5-aminopentylcarbamate for 3-phenyl aniline to afford 164b (668 mg, 99%) as a light yellow oil. LRMS (ESI): (calc) 397.4; (found) 398.2 (MH)⁺.

### Step 2: N-(5-aminopentyl)-2-(4-nitrophenylthio)acetamide (165b)

Following the same procedure as described for compound 2 (step 2, scheme 1, example 1) but substituting 164b for 1 to afford 165b (500 mg, 99%) as a viscous yellow oil. LRMS (ESI): (calc) 297.4; (found) 298.2 (MH)⁺.

### Step 3: 4-(dimethylamino)-N-(5-(2-(4-nitrophenylthio)acetamido)pentyl)benzamide (168b)

Following the same procedure as described for compound 1 (scheme 1, example 1) but substituting 4-(dimethylamino)benzoic acid for N-Boc-caproic acid and amine 165b for 3-phenyl aniline to afford 168b (98 mg, 26%) as a yellow solid. LRMS (ESI): (calc) 444.6; (found) 445.2 (MH)⁺.

### Step 4: N-(5-(2-(4-aminophenylthio)acetamido)pentyl)-4-(dimethylamino)benzamide (169b)

Following the same procedure as described for compound 163a (scheme 20, example 79a) but substituting 168b for 162a to afford 169b (11 mg, 12%) as a light yellow solid.

(MeOD-d4) δ (ppm) ¹H: 7.75-7.70 (m,2H), 7.24-7.20 (m,2H), 6.77-6.72 (m,2H), 6.69-6.63 (m,2H), 3.40-3.32 (m,4H), 3.16 (t, J=6.7 Hz, 2H), 3.04 (s,6H), 1.66-1.54 (m,2H), 1.52-1.42 (m,2H), 1.34-1.24 (m,2H). LRMS (ESI): (calc) 414.6; (found) 415.5 (MH)⁺.

### EXAMPLE 80c,d,e

Example 80c,d,e describe the preparation of compound 168c,d,e using the same procedures as described for compound 168b (steps 1 to 3) in Example 80b. Characterization data are presented in a Table 10.

TABLE 10

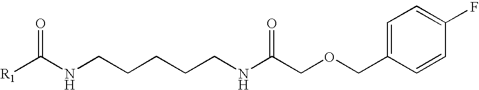

| Ex | Cpd | R₁ | Name | Characterization | Scheme |
|---|---|---|---|---|---|
| 80c | 168c | 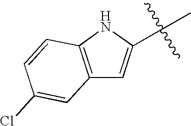 | 5-chloro-N-(5-(2- (4- flurobenzyloxy) acetamido)pentyl )-1H-indole-2- carboxamide | (MeOD-d4) δ (ppm) ¹H: 7.60 (dd, J = 2.0, 0.6 Hz, 1H), 7.45-7.35 (m, 3H), 7.20 (dd, J =8.8, 2.0 Hz, 1H), 7.12-7.03 (m, 3H), 4.54 (s, 2H), 3.94 (s, 2H), 3.43 (t, J = 7.0 Hz, 2H), 3.29 (t, J = 7.0 Hz, 2H), 1.75-1.56 (m, 4H), 1.50-1.40 (m, 2H) LRMS (ESI): (calc) 445.9; | |
| # (found) 446.2 (MH)⁺. | 21 (steps 1-3) | | | | |
| 80d | 168d | 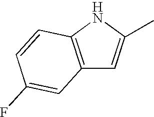 | 5-fluoro-N-(5-(2- (4- flourobenzyloxy) acetamido)pentyl )-1H-indole-2- carboxamide | (MeOD-d4) δ (ppm) ¹H: 7.45-7.41 (m, 1H), 7.39-7.34 (m, 2H), 7.29-7.25 (m, 1H), 7.10-6.98 (m, 4H), 4.53 (s, 2H), 3.93 (s, 2H), 3.42 (t, J =7.0 Hz, 2H), 3.28 (t, J = 7.0 Hz, 2H), 1.73-1.55 (m, 4H), 1.47-1.39 (m, 2H) LRMS (ESI): (calc) 429.5; (found) 430.2 (MH)⁺. | 21 (steps 1—3) |
| 80e | 168e | 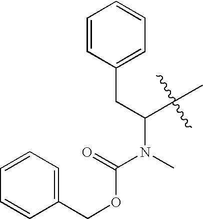 | (S)-benzyl 1-(5- (2-(4- fluorobenzyloxy) acetamido)pentyl amino)-1-oxo-3- phenylpropan-2- yl(methyl)carbamate | (MeOD-d4) δ (ppm) ¹H: 7.46-7.40 (m, 2H), 7.39-7.15 (m,10 H), 7.15-7.07 (m, 2H), 5.15-4.80 (m, 3H), 4.59 (s, 2H), 3.96 (s, 2H), 3.30-3.14 (m, 5H), 3.05-2.95 (m, 1H), 2.88 (s, 3H), 1.57-1.45 (m, 4H), 1.35-1.25 (m, 2H) LRMS (ESI): (calc) 563.7; (found) | |
| # 564.4 (MH)⁺. | 21 (steps 1-3) | | | | |

### EXAMPLE 80f

Example 80f describe the preparation of compound 169c using the same procedures as described for compound 169b in Example 80b. Characterization data are presented in a Table 11.

TABLE 11

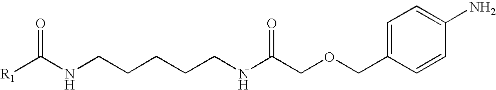

| Ex | Cpd | R₁ | Name | Characterization | Scheme |
|---|---|---|---|---|---|
| 81f | 169c | 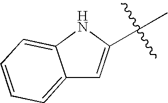 | N-(5-(2-(4- aminophenylthio) acetamido)pentyl )-1H-indole-2- carboxamide | (MeOD-d4) δ (ppm) ¹H: 7.60 (d, J = 8.0 Hz, 1H), 7.46 (d, J = 7.4 Hz, 1H), 7.27-7.19 (m, 3H), 7.10-7.04 (m, 2H), 6.68-6.63 (m, 2H), 3.42-3.36 (m, 4H), 3.17 (t, J = 6.7 Hz, 2H), 1.69-1.59 (m, 2H), 1.54-1.43 (m, 2H), 1.36-1.26 (m, 2H) LRMS (ESI): (calc) 409.5; (found) | |
| # 411.1 MH⁺. | 21 (steps 1-4 (ex 80b)) | | | | |

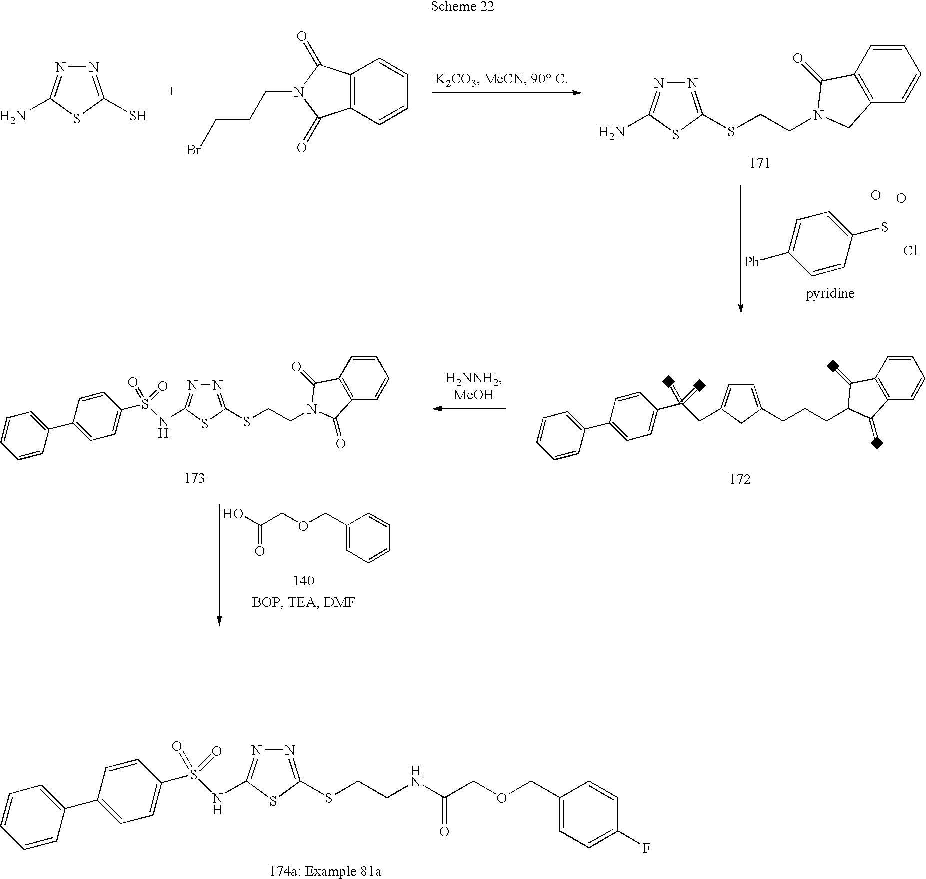

### EXAMPLE 81a

### N-(2-(5-(biphenyl-4-ylsulfonamido)-1,3,4-thiadiazol-2-ylthio)ethyl)-2-(4-fluorobenzyloxy)acetamide (174a)

### Step 1: 2-(2-(5-amino-1,3,4-thiadiazol-2-ylthio)ethyl)isoindoline-1,3-dione (171)

To a stirred solution of 5-amino-1,3,4-thiadiazole-2-thiol (600 mg, 4.50 mmol) in solvent (acetonitrile, dimethylformamide or acetone) (15 mL) at room temperature was added 2-(3-bromopropyl)isoindoline-1,3-dione (1.26 g, 4.95 mmol) and K₂CO₃ (3.11 g, 22.5 mmol). The resulting solution was heated to 90° C. for 30 minutes prior to cooling, dilution with brine, adjustment to pH=13 with NaOH, and extraction with ethyl acetate. The combined organic extracts were dried (Na₂SO₄), filtered, and concentrated. The residue was triturated from 20% MeOH in EtOAc with hexanes to afford 171 (350 mg, 25%) as a light yellow solid. LRMS (ESI): (calc) 306.4; (found) 307.2 (MH)⁺.

### Step 2: N-(5-(2-(1,3-dioxoisoindolin-2-yl)ethylthio)-1,3,4-thiadiazol-2-yl)biphenyl-4-sulfonamide (172)

Following the same procedure as described for compound 54 (scheme 2, example 47) but substituting amine 171 for amine 2 and biphenyl-4-sulfonyl chloride for 3-bromo-propionyl chloride to afford 172 (226 mg, 38%) as a light yellow solid. LRMS (ESI): (calc.) 522.6; (found) 523.5 (MH)⁺.

### Step 3: N-(5-(2-aminoethylthio)-1,3,4-thiadiazol-2-yl)biphenyl-4-sulfonamide (173)

To a stirred solution of N-(5-(2-(1,3-dioxoisoindolin-2-yl)ethylthio)-1,3,4-thiadiazol-2-yl)biphenyl-4-sulfonamide 172 (226 mg, 0.432 mmol) in methanol (4 mL) was added hydrazine hydrate (0.042 mL, 0.865 mmol) at room temperature. The resulting solution was stirred for 16 hours prior to removal of the white precipitate by filtration, and evaporation of the filtrate to afford 173 (170 mg, 99%) as a white solid which was used in the subsequent step without further purification. LRMS (ESI): (calc.) 392.5; (found) 393.1 (MH)⁺.

### Step 4: N-(2-(5-(biphenyl-4-ylsulfonamido)-1,3,4-thiadiazol-2-ylthio)ethyl)-2-(4-fluorobenzyloxy)acetamide (174a)

Following the same procedure as described for compound 1 (scheme 1, example 1) but substituting 2-(4-fluorobenzyloxy)acetic acid 140 for N-Boc-caproic acid and amine 173 for 3-phenyl aniline to afford 174a (42 mg, 17%) as a white solid. (MeOD-d4) δ (ppm) ¹H: 8.20-7.86 (m,3H), 7.68-7.63 (m,2H), 7.60-7.55 (m,2H), 7.46-7.38 (m,2H), 7.38-7.30 (m,3H), 7.06-6.99 (m,2H), 4.47 (s,2H), 3.85 (s,2H), 3.51 (t, J=6.5 Hz, 2H), 3.21 (t, J=6.5 Hz, 2H). LRMS (ESI): (calc) 558.7; (found) 559.2 (MH)⁺.

### EXAMPLE 81b,c

Example 81b,c describe the preparation of compound 174b,c using the same procedures as described for compound 174a in Example 81a. Characterization data are presented in a Table 12.

TABLE 12

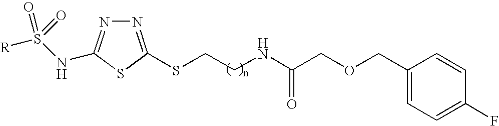

| Ex | Cpd | n | R | Name | Characterization | Scheme |
|---|---|---|---|---|---|---|
| 81b | 174b | 2 | 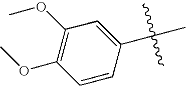 | N-(3-(5-(3,4- dimethoxyphenylsulfona mido)-1,3,4-thiadiazoi-2- ylthio)propyl)-2-(4- fluorobenzyloxy)acetamide | (MeOD-d4) δ (ppm) ¹H: 7.48 (dd, J = 8.4, 2.2 Hz, 1H), 7.46-7.40 (m,3H), 7.14-7.07 (m, 2H), 7.02 (d, J = 8.4 Hz, 1H), 4.59 (s, 2H), 3.96 (s, 2H), 3.88 (s, 3H), 3.87 (s, 3H), 3.40-3.35 (m, 2H), 3.11 (t, J = 7.2 Hz, 2H), 1.96-1.88 (m, 2H) LRMS (ESI): | |
| # (calc) 556.7; (found) 557.4 (MH)⁺. | 22 | | | | | |
| 81c | 174c | 3 | 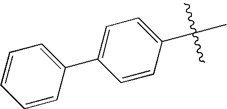 | N-(4-(5-(biphenyl-4- ylsulfonamido)-1,3,4- thiadiazol-2-ylthio)butyl)- 2-(4- fluorobenzyloxy)acetamide | (MeOD-d4) δ (ppm) ¹H: 8.22-7.86 (m, 3H), 7.71-7.58 (m, 4H), 7.46-7.32 (m, 5H), 7.10-7.02 (m, 3H), 4.52 (s, 2H), 3.89 (s, 2H), 3.21 (t, J = 6.3 Hz, 2H), 3.06 (t, J = 7.0 Hz, 2H), 1.70-1.56 (m, 4H) LRMS (ESI): (calc) 586.7; (found) 587.0 (MH)⁺. | 22 |

### EXAMPLE 81e,f

Example 81e describe the preparation of compound 174e using the same procedures as described for compound 174a in Example 81a. Characterization data is presented in a Table 13.

Example 81f describe the preparation of compound 174f using the same procedures as described for compound 7 in Example 2. Characterization data is presented in a Table 13.

TABLE 13

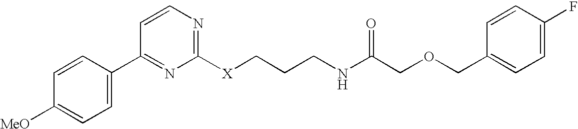

| Ex | Cpd | X | Name | Characterization | Scheme |
|---|---|---|---|---|---|
| 81e | 174e | S | 2-(4-fluorobenzyloxy)-N- | (MeOD-d4) δ (ppm) ¹H: 8.57 (d, J= | 22 (steps 1,3,4) |
| | | | (3-(4-(4- | 6.1 Hz, 1H), 8.32-8.26 (m, 2H), 7.83 | |
| | | | methoxyphenyl)pyrimidin- | (d, J = 6.1 Hz, 1H), 7.44-7.38 | |
| | | | 2-ylthio)propyl)acetamide | (m, 2H), 7.18-7.14 (m, 2H), 7.11- | |
| | | | | 7.05 (m, 2H), 4.59 (s, 2H), 3.97 | |
| | | | | (s, 2H), 3.93 (s, 3H), 3.48 (t, J = 6.7 | |
| | | | | Hz, 2H), 3.44 (t, J = 7.4 Hz, 2H), | |
| | | | | 2.13-2.05 (m, 2H) | |
| | | | | LRMS (ESI): (calc) 441.5; (found) | |
| | | | | 442.5 (MH)⁺. | |
| B1f | 174f | S═O | 2-(4-fluorobenzyloxy)-N- | (MeOD-d4) δ (ppm) ¹H: 8.86 (d, J = | 22 (steps 1,3,4) |
| | | | (3-(4-(4- | 5.5 Hz, 1H), 8.32-8.24 (m, 2H), 8.01 | 1 (step 6 (ex 2)) |
| | | | methoxyphenyl)pyrimidin- | (d, J = 5.5 Hz, 1H), 7.44-7.35 | |
| | | | 2- | (m, 2H), 7.15-7.04 (m, 4H), 4.54 | |
| | | | ylsulfinyl)propyl)acetamide | (s, 2H), 3.91 (s, 5H), 3.42-3.35 | |
| | | | | (m, 3H), 3.25-3.17 (m, 1H), 2.20- | |
| | | | | 2.05 (m, 1H), 1.90-1.77 (m, 1H) | |
| | | | | LRMS (ESI): (calc) 457.5; (found) | |
| | | | | 458.3 (MH)⁺. | |

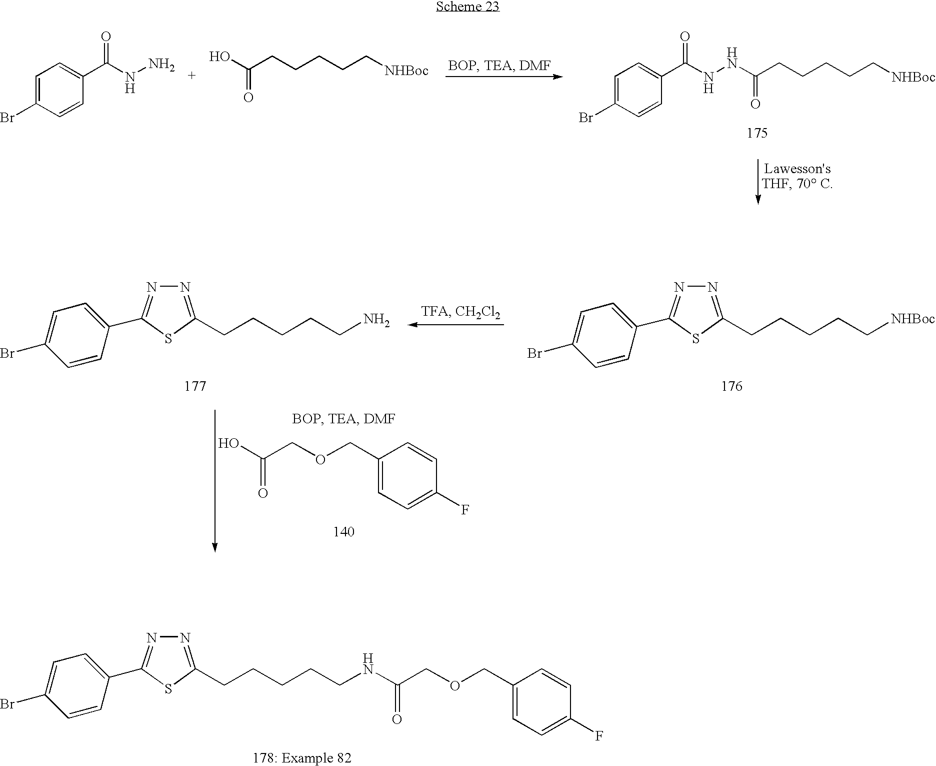

### EXAMPLE 82

### N-(5-(5-(4-bromophenyl)-1,3,4-thiadiazol-2-yl)pentyl)-2-(4-fluorobenzyloxy)acetamide (178)

### Step 1: tert-butyl 6-(2-(4-bromobenzoyl)hydrazinyl)-6-oxohexylcarbamate (175)

Following the same procedure as described for compound 1 (scheme 1, example 1) but substituting 4-bromobenzohydrazide for 3-phenyl aniline to afford 175 (1.03 mg, 74%) as a white solid. LRMS (ESI): (calc) 428.4; (found) 429.2 (MH)⁺.

### Step 2: tert-butyl 5-(5-(4-bromophenyl)-1,3,4-thiadiazol-2-yl)pentylcarbamate (176)

To a stirred solution of tert-butyl 6-(2-(4-bromobenzoyl)hydrazinyl)-6-oxohexylcarbamate 175 (1.03 g, 2.40 mmol) in tetrahydrofuran (20 mL) at room temperature was added Lawesson's reagent (1.07 g, 2.64 mmol). The resulting solution was heated to 70° C. for 2 hours prior to cooling, removal of the solvent, and direct purification of the residue by silica gel column chromatography using EtOAc (0-100%) in hexanes to afford 176 (378 mg, 37%) as a yellow solid. LRMS (ESI): (calc) 426.4; (found) 427.6 (MH)⁺.

### Step 3: 5-(5-(4-bromophenyl)-1,3,4-thiadiazol-2-yl)pentan-1-amine (177)

Following the same procedure as described for compound 2 (step 2, scheme 1, example 1) but substituting 176 for 1 to afford 177 (275 mg, 95%) as light yellow solid. LRMS (ESI): (calc) 326.4; (found) 327.2 (MH)⁺.

### Step 4: N-(5-(5-(4-bromophenyl)-1,3,4-thiadiazol-2-yl)pentyl)-2-(4-fluorobenzyloxy)acetamide (178)

Following the same procedure as described for compound 1 (scheme 1, example 1) but substituting 2-(4-fluorobenzyloxy)acetic acid 140 for N-Boc-caproic acid and amine 177 for 3-phenyl aniline to afford 178 (21 mg, 5%) as a light yellow solid. (MeOD-d4) δ (ppm) ¹H: 7.92-7.87 (m,2H), 7.75-7.70 (m,2H), 7.44-7.38 (m,2H), 7.13-7.07 (m,2H), 4.57 (s,2H), 3.95 (s,2H), 3.28 (t, J=7.0 Hz, 2H), 3.20 (t, J=7.6 Hz, 2H), 1.96-1.86 (m,2H), 1.67-1.58 (m,2H), 1.53-1.43 (m,2H). LRMS (ESI): (calc) 492.4; (found) 493.4 (MH)⁺.

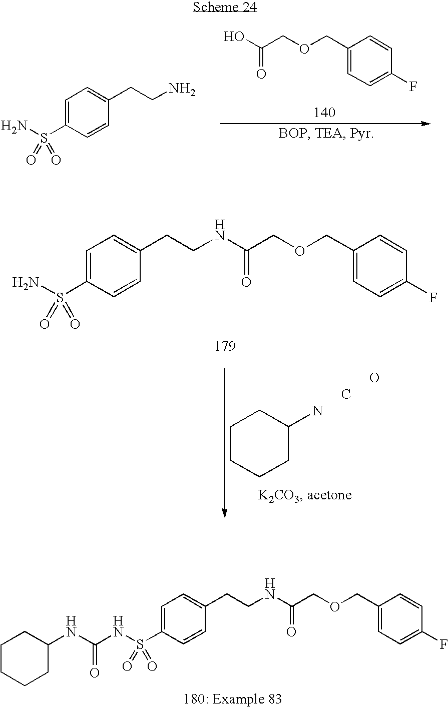

### EXAMPLE 83

### N-(4-(N-(cyclohexylcarbamoyl)sulfamoyl)phenethyl)-2-(4-fluorobenzyloxy)acetamide (180)

### Step 1: 2-(4-fluorobenzyloxy)-N-(4-sulfamoylphenethyl)acetamide (179)

Following the same procedure as described for compound 1 (scheme 1, example 1) but substituting 2-(4-fluorobenzyloxy)acetic acid 140 for N-Boc-caproic acid and 4-(2-aminoethyl)benzenesulfonamide for 3-phenyl aniline to afford 179 (275 mg, 55%) as a white solid. (MeOD-d4) δ (ppm) ¹H: 7.88-7.83 (m,2H), 7.46-7.36 (m,4H), 7.16-7.10 (m,2H), 4.45 (s,2H), 3.93 (s,2H), 3.54 (t, J=7.6 Hz, 2H), 2.94 (t, J=7.0 Hz, 2H). LRMS (ESI): (calc) 366.4; (found) 367.1 (MH)⁺.

### Step 2: N-(4-(N-(cyclohexylcarbamoyl)sulfamoyl)phenethyl)-2-(4-fluorobenzyloxy)acetamide (180)

To a stirred solution of 2-(4-fluorobenzyloxy)-N-(4-sulfamoylphenethyl)acetamide 179 (100 mg, 0.273 mmol) in acetone (2 mL) at room temperature was added K₂CO₃ (75 mg, 0.546 mmol), and the resulting solution stirred for 5 minutes prior to the addition of cyclohexylisocyanate (0.05 mL, 0.409 mmol). The resulting solution was heated to reflux for 16 hours prior to cooling, dilution with brine, acidification to pH=1 with HCl, and extraction with ethyl acetate. The combined organic extracts were dried (Na₂SO₄), filtered, and concentrated. The residue was then purified by acid-base extraction to afford 180 (7 mg, 5%) as a white solid. (MeOD-d4) δ (ppm) ¹H: 7.95-7.90 (m,2H), 7.50-7.46 (m,2H), 7.41-7.35 (m,2H), 7.16-7.09 (m,2H), 4.54 (s,2H), 3.93 (s,2H), 3.55 (t, J=7.2 Hz, 2H), 3.50-3.38 (m,1H), 2.96 (t, J=6.8 Hz, 2H), 1.82-1.55 (m,6H), 1.39-1.14 (m,4H). LRMS (ESI): (calc) 491.6; (found) 492.3 (MH)⁺.

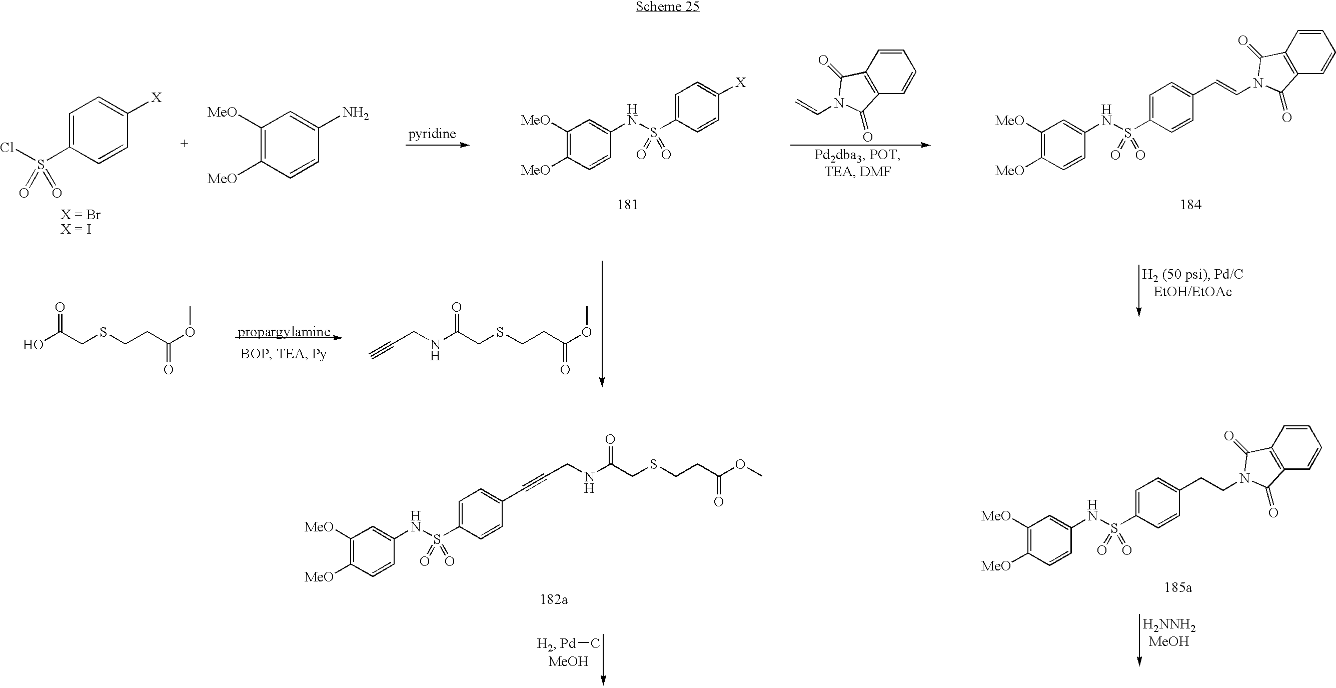

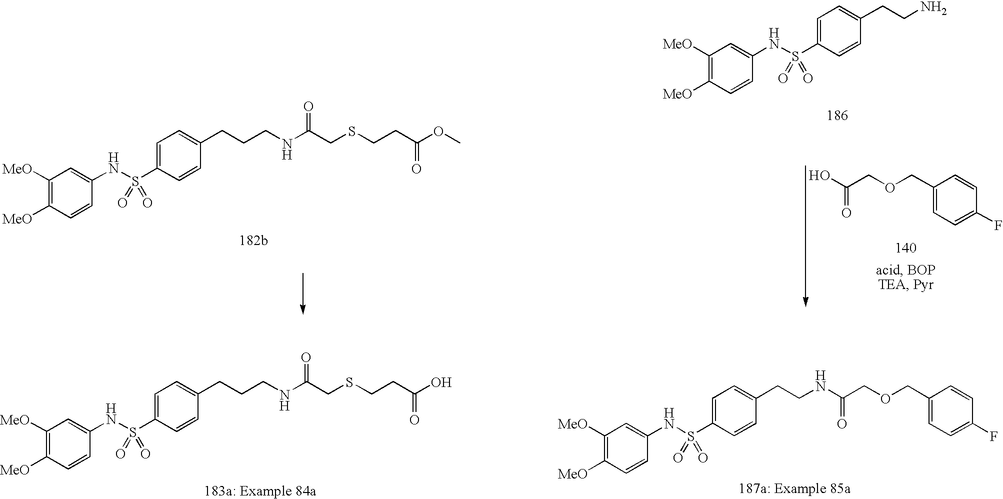

### EXAMPLE 84a

### 3-(2-(3-(4-(N-(3,4-dimethoxyphenyl)sulfamoyl)phenyl)propylamino)-2-oxoethylthio)propanoic (183a)

### Step 1: N-(3,4-dimethoxyphenyl)-4-iodobenzenesulfonamide (181)

To a stirred solution of 4-iodobenzene-1-sulfonyl chloride (1.00 g, 3.31 mmol) in pyridine (8 mL) at room temperature was added 3,4-dimethoxyaniline (507 mg, 3.31 mmol). The resulting solution was stirred for 2 hours prior to removal of the solvent, and direct purification of the residue by silica gel column chromatography using MeOH (10%) in EtOAc to afford 181 (769 mg, 55%) as a light pink solid. LRMS (ESI): (calc) 419.2; (found) 420.2 (MH)⁺.

### Intermediate: methyl 3-(2-oxo-2-(prop-2-ynylamino)ethylthio)propanoate

Following the same procedure as described for compound 1 (scheme 1, example 1) but substituting 2-(3-methoxy-3-oxopropylthio)acetic acid for N-Boc-caproic acid and propargylamine for 3-phenyl aniline to afford methyl 3-(2-oxo-2-(prop-2-ynylamino)ethylthio)propanoate (633 mg, 69%) as a white solid. LRMS (ESI): (calc) 215.3; (found) 216.2 (MH)⁺.

### Step 2: methyl 3-(2-(3-(4-(N-(3,4-dimethoxyphenyl)sulfamoyl)phenyl)prop-2-ynylamino)-2-oxoethylthio)propanoate (182a)

To a stirred solution of N-(3,4-dimethoxyphenyl)-4-iodobenzenesulfonamide 181 (560 mg, 1.34 mmol) in acetonitrile (7 mL) at room temperature was added methyl 3-(2-oxo-2-(prop-2-ynylamino)ethylthio)propanoate (316 mg, 1.47 mmol), Pd Cl₂(PPh₃)₂ (47 mg, 0.067 mmol), copper(I) iodide (26 mg, 0.134 mmol), and triethylamine (0.28 mL, 2.01 mmol). The resulting solution was stirred for 2 hours prior to dilution with brine, adjustment to pH=1 with HCl, and extraction with ethyl acetate. The combined organic extracts were dried (Na₂SO₄), filtered, and concentrated. The residue was purified by silica gel column chromatography using EtOAc (0-75%) in hexanes to afford 182a (336 mg, 49%) as an orange oil. LRMS (ESI): (calc) 506.6; (found) 507.2 (MH)⁺.

### Step 3: methyl 3-(2-(3-(4-(N-(3,4-dimethoxyphenyl)sulfamoyl)phenyl)propylamino)-2-oxoethylthio)propanoate (182b)

Following the same procedure as described for compound 74 (scheme 5, example 55) but substituting 182a for 73 to afford 182b (120 mg, 56%) as a yellow oil. (MeOD-d4) δ (ppm) ¹H: 7.67-7.62 (m,2H), 7.35-7.31 (m,2H), 6.77 (d, J=8.6 Hz, 1H), 6.70 (d, J=2.3 Hz, 1H), 6.60 (dd, J=8.6,2.3 Hz, 1H), 3.75 (s,3H), 3.71 (s,3H), 3.65 (s,3H), 3.26-3.19 (m,4H), 2.85 (t, J=6.8 Hz, 2H), 2.72-2.65 (m,4H), 1.87-1.77 (m,2H). LRMS (ESI): (calc) 510.6; (found) 511.3 (MH)⁺.

### Step 4: 3-(2-(3-(4-(N-(3,4-dimethoxyphenyl)sulfamoyl)phenyl)propylamino)-2-oxoethylthio)propanoic (183a)

Following the same procedure as described for compound 5 (scheme 1, example 1) but substituting 182b for 4 to afford 183a (79 mg, 80%) as a yellow oil. (MeOD-d4) δ (ppm) ¹H: 7.67-7.61 (m,2H), 7.37-7.31 (m,2H), 6.78 (d, J=8.8 Hz, 1H), 6.69 (d, J=2.3 Hz, 1H), 6.59 (dd, J=8.4,2.3 Hz, 1H), 3.76 (s,3H), 3.72 (s,3H), 3.60-3.20 (m,4H), 2.84 (t, J=6.8 Hz, 2H), 2.74-2.62 (m,4H), 1.88-1.80 (m,2H). LRMS (ESI): (calc) 496.6; (found) 497.2 (MH)⁺.

### EXAMPLE 84b,c,d,e,f

Example 84b,c,d,e,f describe the preparation of compound 182b,c,d,e,f using the same procedures as described for compound 182a in Example 84a or mention in the table 14. Characterization data are presented in a Table 14.

TABLE 14

| | | | | | Scheme |
|---|---|---|---|---|---|
| Ex | Cpd | structure | Name | Characterization | (step) |
| 84b | 182b | 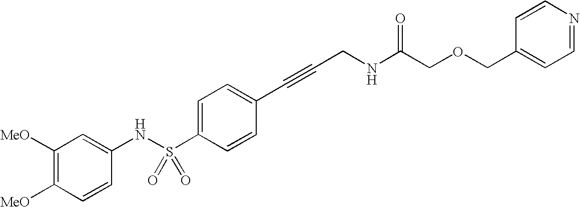 | N-(3-(4-(N-(3,4- dimethoxyphenyl )sulfamoyl)phenyl) prop-2-ynyl)- 2-(pyridin-4- ylmethoxy)acetamide | (DMSO-d6) δ (ppm) ¹H: 10.01 (s, 1H), 8.53 (t, J =5.7 Hz, 1H), 7.69 (d, J =8.2 Hz, 3H), 7.59 (d, J =8.2 Hz, 3H), 6.82 (d, J =8.4 Hz, 1H), | |
| # 6.69 (s, 1H), 6.58-6.50 (m, 1H), 4.61 (br s, 2H), 4.21 (d, J = 5.7 Hz, 2H), 4.07 (s, 2H), 3.69 (s, 3H), 3.66 (s, 3H) LRMS (ESI): (calc) 495.6; (found) 496.4 MH⁺. | 25 (1-2 (ex 84a)) 25 (1-2(ex 84a)) | | | | |
| 84c | 182c | 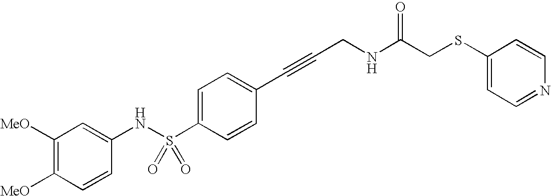 | N-(3-(4-(N-(3,4- dimethoxyphenyl )sulfamoyl)phen yl)prop-2-ynyl)- 2-(pyridin-4- ylthio)acetamide | (MeOD-d4) δ (ppm) ¹H: 8.33 (br s, 2H), 7.69-7.64 (m, 2H), 7.48-7.43 (m, 2H), 7.36 (d, J = 5.5 Hz, 2H), 6.80 (d, J = 8.6 Hz, 1H), 6.70 (d, | |
| # J = 2.5 Hz, 1H), 6.57 (dd, J =8.6, 2.3 Hz, 1H), 4.24 (s, 2H), 3.88 (s, 2H), 3.78 (s, 3H), 3.74 (s, 3H) LRMS (ESI): (calc) 497.6; (found) 498.2 MH⁺. | 25 (1-2 (ex 84a)) | | | | |
| 84d | 182d | 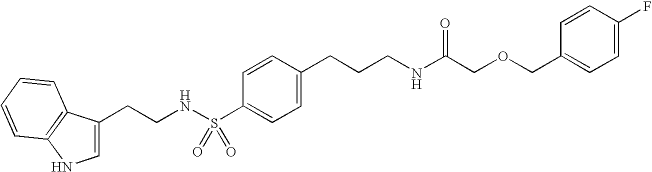 | N-(3-(4-(N-(2- (1H-indol-3- yl)ethyl)sulfamoyl )phenyl)propyl)- 2-(4- fluorobenzyloxy) acetamide | (MeOD-d4) δ (ppm) ¹H: 7.74-7.70 (m, 2H), 7.45-7.30 (m, 6H), 7.13-7.05 (m, 3H), 7.02-6.95 (m, 2H), 4.57 (s, 2H), 3.93 (s, 2H), 3.29 (t, | |
| # J =7.0 Hz, 2H), 3.16 (t, J =7.8 Hz, 2H), 2.88 (t, J =7.6 Hz, 2H), 2.72 (t, J =8.2 Hz, 2H), 1.92-1.82 (m, 2H) LRMS (ESI): (calc) 523.6; (found) 524.1 (MH)⁺. | 25(-3(ex 84a)) | | | | |
| 84e | 182e | 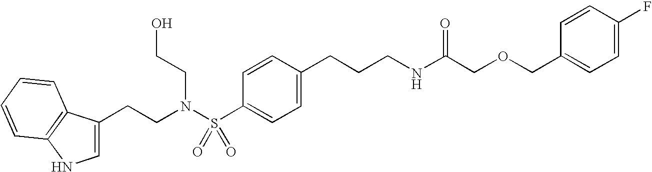 | N-(3-(4-(N-(2- (1H-indol-3- yl)ethyl)-N-(2- hydroxyethyl)sulamoyl) phenyl)propyl )-2-(4- fluorobenzyloxy) acetamide | (MeOD-d4) δ (ppm) ¹H: 10.21 (br s, 1H), 7.90 (t, J = 5.7 Hz, 1H), 7.73-7.69 (m, 2H), 7.55-7.51 (m, 1H), 7.40-7.30 (m, 5H), | |
| # 7.12-6.95 (m, 5H), 4.53 (s, 2H), 3.91 (s, 2H), 3.68 (t, J =6.3 Hz, 2H), 3.48-3.41 (m, 2H), 3.30-3.22 (m, 4H), 3.06-2.98 (m, 2H), 2.70-2.62 (m, 2H), 1.87-1.77 (m,2H) LRMS (ESI): (calc) 567.7; (found) 568.4 MH⁺. | 25 (1 (ex 84a)) 22 (1 (ex 81a)) 25 (2-3 (ex 84a)) | | | | |
| 84f | 182f | 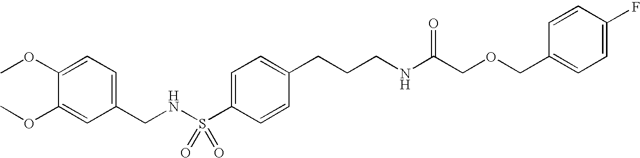 | N-(3-(4-(N-(3,4- dimethoxybenzyl )sulfamoyl)phenyl) propyl)-2-(4- fluorobenzyloxy) acetamide | (MeOD-d4) δ (ppm) ¹H: 7.72-7.68 (m, 2H), 7.47-7.41 (m, 2H), 7.35-7.30 (m, 2H), 7.14-7.07 (m, 2H), 6.82-6.77 (m, 1H), 6.76-6.71 (m, 2H), 4.59 (s, 2H), 4.04 (s, 2H), | |
| # 3.96 (s, 2H), 3.77 (s, 3H), 3.71 (s, 3H), 3.28 (t, J =7.0 Hz, 2H), 2.69 (t, J =7.8 Hz, 2H), 1.90-1.80 (m, 2H) LRMS (ESI): (calc) 530.6; (found) 531.0 (MH)⁺. | 25 (1-3 (ex 84a)) | | | | |

### EXAMPLE 85a

### N-(4-(N-(3,4-dimethoxyphenyl)sulfamoyl)phenethyl)-2-(4-fluorobenzyloxy)acetamide (187a)

### Step 1: 4-bromo-N-(3,4-dimethoxyphenyl)benzenesulfonamide (181)

Following the same procedure as described for compound 181 (X═I) (scheme 25, example 84a) but substituting 4-bromobenzene-1-sulfonyl chloride for 4-iodobenzene-1-sulfonyl chloride to afford 181 (X═Br) (887 mg, 90%) as a light pink solid. LRMS (ESI): (calc) 372.2; (found) 373.5 (MH)⁺.

### Step 2: (E)-N-(3,4-dimethoxyphenyl)-4-(2-(1,3-dioxoisoindolin-2 yl)vinyl)benzenesulfonamide (184)

To a stirred solution of 4-bromo-N-(3,4-dimethoxyphenyl)benzenesulfonamide 181 (887 mg, 2.39 mmol) in dimethylformamide (20 mL) at room temperature was added vinyl phthalamide (414 mg, 2.39 mmol), Pd₂ dba₃ (131 mg, 0.143 mmol), tri-o-tolylphosphine (87 mg, 0.287 mmol), and triethylamine (0.83 mL, 5.98 mmol). The resulting solution was heated to 100° C. for 16 hours prior to cooling, dilution with brine, adjustment to pH=2 with HCl, and extraction with ethyl acetate. The combined organic extracts were dried (Na₂SO₄), filtered, and concentrated. The residue was purified by silica gel column chromatography using EtOAc (0-100%) in hexanes to afford 184 (285 mg, 26%) as a light yellow solid. LRMS (ESI): (calc) 464.5; (found) 465.2 (MH)⁺.

### Step 3: N-(3,4-dimethoxyphenyl)-4-(2-(1,3-dioxoisoindolin-2-yl)ethyl)benzenesulfonamide (185a)

To a solution of (E)-N-(3,4-dimethoxyphenyl)-4-(2-(1,3-dioxoisoindolin-2-yl)vinyl)benzenesulfonamide 184 (285 mg, 0.614 mmol) in ethanol/ethyl acetate (1:1, 20 mL) was added 10% Pd/C (83 mg). This solution was then placed under 50 p.s.i. of hydrogen in a Parr shaker for 7 hours. Subsequently, the solution was filtered through a pad of celite to remove the catalyst, and the filtrate evaporated to afford 185a (190 mg, 67%) as a light yellow solid which was used in the subsequent reaction without further purification. LRMS (ESI): (calc) 466.5; (found) 467.2 (MH)⁺.

### Step 4: 4-(2-aminoethyl)-N-(3,4-dimethoxyphenyl)benzenesulfonamide (186)

Following the same procedure as described for compound 173 (scheme 22, example 81a) but substituting 185a for 172 to afford 186 (100 mg, 73%) as a light yellow foam. LRMS (ESI): (calc) 336.4; (found) 337.4 (MH)⁺.

### Step 5: N-(4-(N-(3,4-dimethoxyphenyl)sulfamoyl)phenethyl)-2-(4-fluorobenzyloxy)acetamide (187a)

Following the same procedure as described for compound 1 (scheme 1, example 1) but substituting 2-(4-fluorobenzyloxy)acetic acid 140 for N-Boc-caproic acid and 186 for 3-phenyl aniline to afford 187a (30 mg, 2%) as a white solid. (MeOD-d4) δ (ppm) ¹H: 7.69-7.64 (m,2H), 7.40-7.32 (m,4H), 7.15-7.07 (m,2H), 6.77 (d, J=8.6 Hz, 1H), 6.71 (d, J=2.5 Hz, 1H), 6.58 (dd, J=8.4,2.3 Hz, 1H), 4.50 (s,2H), 3.88 (s,2H), 3.75 (s,3H), 3.73 (s,3H), 3.50 (t, J=7.0 Hz, 2H), 2.90 (t, J=7.2 Hz, 2H). LRMS (ESI): (calc) 502.6; (found) 503.2 (MH)⁺.

### EXAMPLE 85b

Example 85b describe the preparation of compound 185b using the same procedures as described for compound 185a in Example 85a. Characterization data are presented in a Table 15.

TABLE 15

| Ex | Cpd | structure | Name | Characterization | Scheme |
|---|---|---|---|---|---|
| 85b | 185 b | 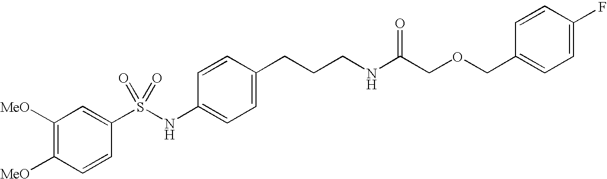 | N-(3-(4-(3,4- dimethoxyphenyl sulfonamido)phenyl)propyl)-2-(4- fluorobenzyloxy) actamime | (MeOD-d4) δ (ppm) ¹H: 7.46-7.41 (m, 2H), 7.34 (dd, J = 8.4, 2.2 Hz, 1H), 7.19 (d, J = 2.2 Hz, 1H), 7.15-7.07 (m, 4H), 7.04-6.98 (m, 3H), 4.58 (s, | |
| # 2H), 3.93 (s, 2H), 3.87 (s, 3H), 3.77 (s, 3H), 3.24 (t, J = 7.0 Hz, 2H), 2.58 (t, J = 7.8 Hz, 2H), 1.84 1.74 (m, 2H) LRMS (ESI): (calc) 516.6; (found) 517.0 (MH)⁺. | 25 (steps 1-3 (ex 85a)) | | | | |

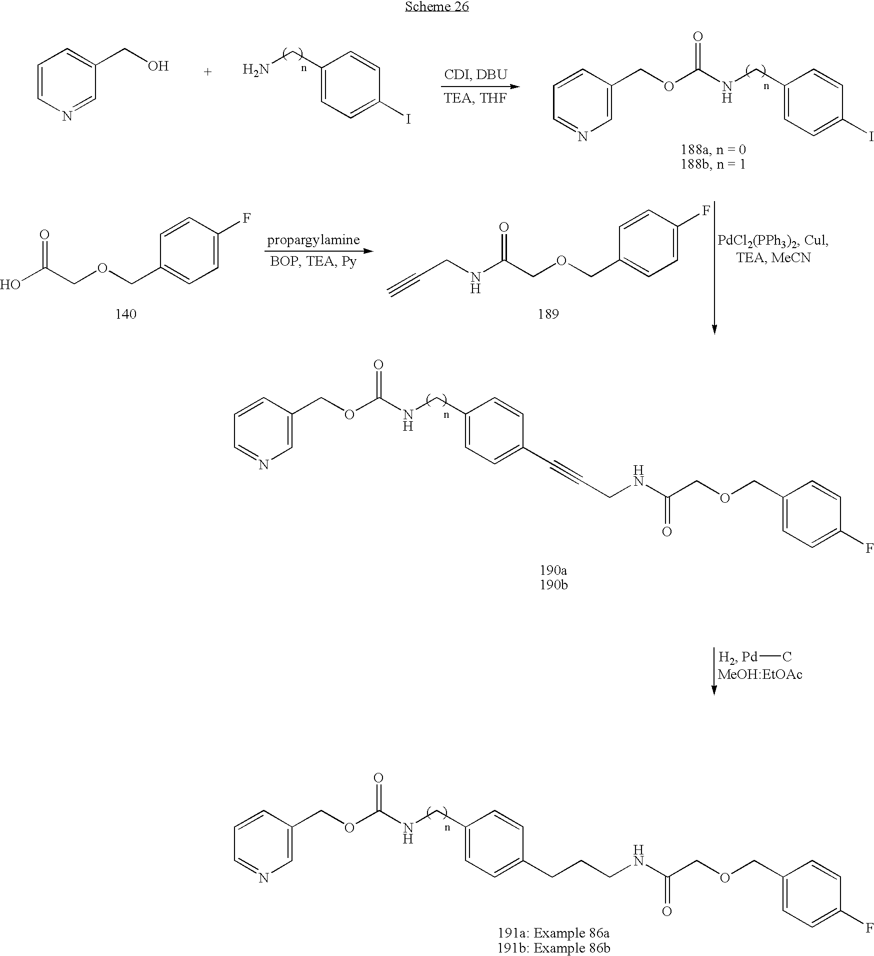

### EXAMPLE 86a

### pyridin-3-ylmethyl 4-(3-(2-(4-fluorobenzyloxy)acetamido)propyl)phenylcarbamate (191a)

### Step 1: pyridin-3-ylmethyl 4-iodophenylcarbamate (188a)

To a stirred solution of carbonyl diimidazole (CDI) (2.69 g, 16.55 mmol) in tetrahydrofuran (12 mL) at 0° C. was added a solution of pyridin-3-ylmethanol (1.61 mL, 16.55 mmol) in tetrahydrofuran (5 mL). The resulting solution was stirred for 1 hour at room temperature prior to the addition of 4-iodoaniline (3.62 g, 16.55 mmol), DBU (2.48 mL, 16.55 mmol) and triethylamine (2.31 mL, 16.55 mmol) dissolved in tetrahydrofuran (10 mL). The resulting solution was then stirred at room temperature for 16 hours prior to dilution with brine, adjustment to pH=13 with NaOH, and extraction with ethyl acetate. The combined organic extracts were dried (Na₂SO₄), filtered, and concentrated. The residue was the taken up in ethyl acetate, and triturated with hexanes to afford 188a (1.27 g, 17%) as a light grey solid. LRMS (ESI): (calc) 354.2; (found) 355.2 (MH)⁺.

### Step 2: 2-(4-fluorobenzyloxy)-N-(prop-2-ynyl)acetamide (189)

Following the same procedure as described for methyl 3-(2-oxo-2-(prop-2-ynylamino)ethylthio)propanoate (scheme 25, example 84a) but substituting 2-(4-fluorobenzyloxy)acetic acid 140 for 2-(3-methoxy-3-oxopropylthio)acetic acid to afford 189 (1.52 g, 62%) as a light yellow oil. LRMS (ESI): (calc) 221.2; (found) 222.6 (MH)⁺.

### Step 3: pyridin-3-ylmethyl 4-(3-(2-(4-fluorobenzyloxy)acetamido)prop-1-ynyl)phenylcarbamate (190a)

Following the same procedure as described for compound 182a (scheme 25, example 84a) but substituting 188a for 181 and 189 for methyl 3-(2-oxo-2-(prop-2-ynylamino)ethylthio)propanoate to afford 190a (270 mg, 62%) as a light grey solid. (MeOD-d4) δ (ppm) ¹H, 8.66 (s,1H), 8.58-8.50 (m,1H), 7.98-7.93 (m,1H), 7.53-7.33 (m,7H), 7.14-7.08 (m,2H), 5.28 (s,2H), 4.62 (s,2H), 4.25 (s,2H), 4.02 (s,2H). LRMS (ESI): (calc) 447.5; (found) 448.2 (MH)⁺.

### Step 4: pyridin-3-ylmethyl 4-(3-(2-(4-fluorobenzyloxy)acetamido)propyl)phenylcarbamate (191a)

Following the same procedure as described for compound 74 (scheme 5, example 55) but substituting 190a for 73 to afford 191a (40 mg, 50%) as a white solid.

(MeOD-d4) δ (ppm) ¹H, 8.70-8.62 (m,1H), 8.57-8.50 (m,1H), 7.98-7.93 (m,1H), 7.53-7.34 (m,5H), 7.17-7.08 (m,4H), 5.26 (s,2H), 4.58 (s,2H), 3.94 (s,2H), 3.28 (t, J=7.0 Hz, 2H), 2.61 (t, J=7.4 Hz, 2H), 1.88-1.79 (m,2H). LRMS (ESI): (calc) 451.5; (found) 452.2 (MH)⁺.

### EXAMPLE 86b

### pyridin-3-ylmethyl 4-(3-(2-(4-fluorobenzyloxy)acetamido)propyl)benzylcarbamate (191b)

### Step 1-4: pyridin-3-ylmethyl 4-(3-(2-(4-fluorobenzyloxy)acetamido)propyl)benzylcarbamate (191b)

Following the same procedure as described for compound 191a (scheme 26, example 88a) but substituting (4-iodophenyl)methanamine for 4-iodoaniline to afford 191b (25 mg, 23%) as a light yellow oil. (MeOD-d4) δ (ppm) ¹H, 8.59 (s,1H), 8.50 (d, J=3.9 Hz, 1H), 7.88 (d, J=7.8 Hz, 1H), 7.47-7.40 (m,3H), 7.24-7.07 (m,6H), 5.18 (s,2H), 4.59 (s,2H), 4.28 (s,2H), 3.94 (s,2H), 3.27 (t, J=7.0 Hz, 2H), 2.63 (t, J=7.8 Hz, 2H), 1.88-1.78 (m,2H). LRMS (ESI): (calc) 465.5; (found) 466.3 (MH)⁺.

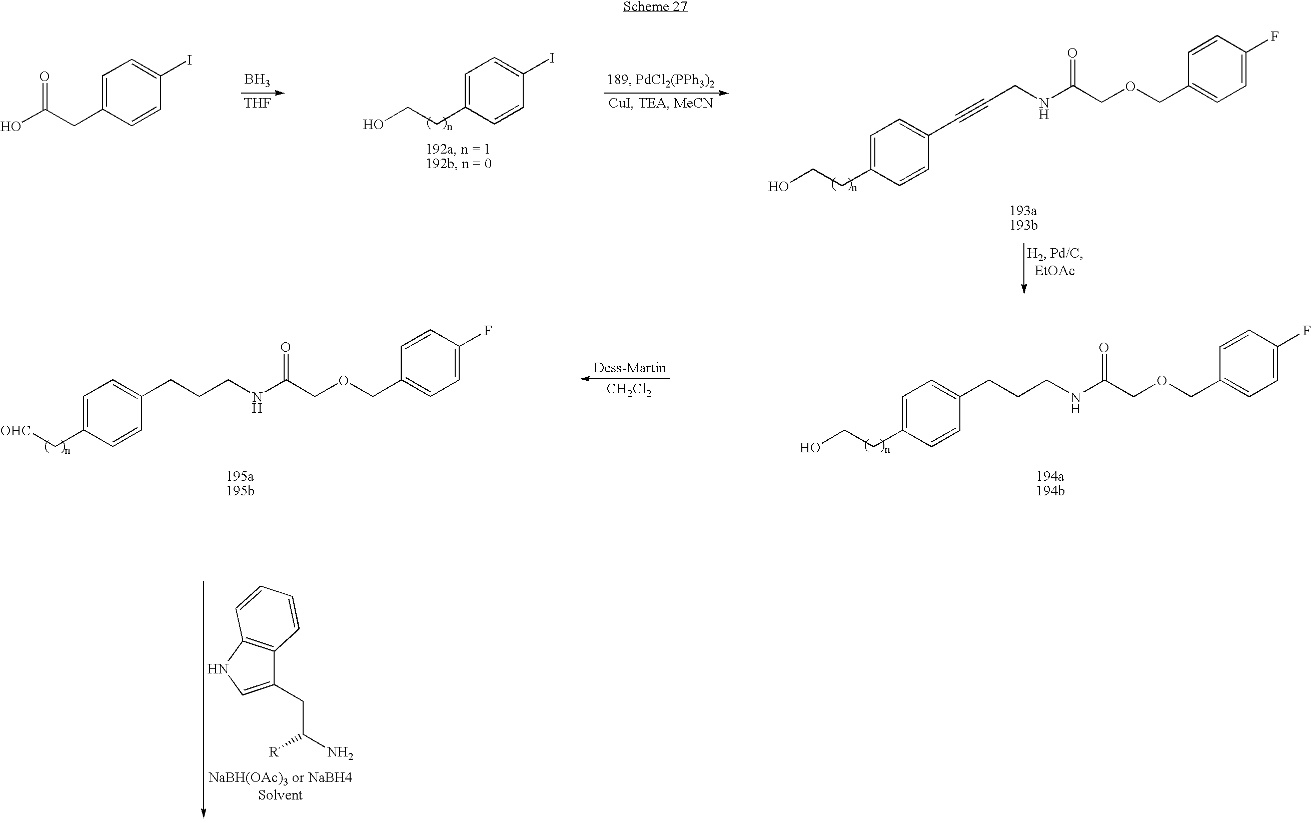

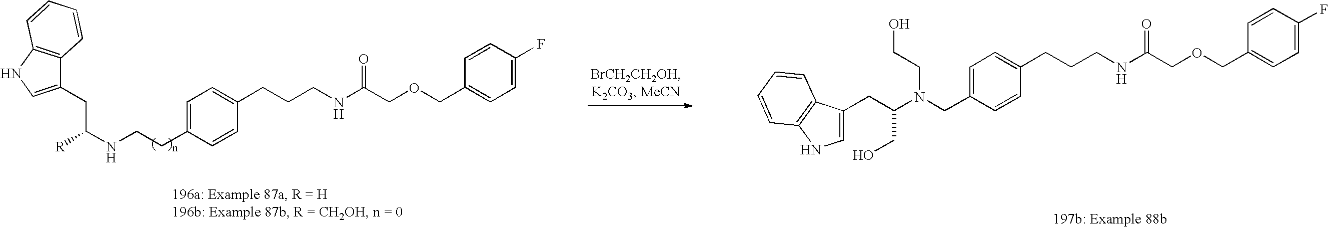

### EXAMPLE 87a

### N-(3-(4-(2-(2-(1H-indol-3-yl)ethylamino)ethyl)phenyl)propyl)-2-(4-fluorobenzyloxy)acetamide (196a)

### Step 1: 2-(4-iodophenyl)ethanol (192a)

To a stirred solution of 2-(4-iodophenyl)acetic acid (2.00 g, 7.63 mmol) in tetrahydrofuran (150 mL) at room temperature was added borane-tetrahydrofuran complex (1.0 M in tetrahydrofuran, 7.63 mL, 7.63 mmol). The resulting solution was stirred for 6 hours prior to dilution with brine, adjustment to pH=13 with NaOH, and extraction with ethyl acetate. The combined organic extracts were dried (Na₂SO₄), filtered, and concentrated to afford 192a (620 mg, 33%) as a light orange solid which was used in the subsequent reaction without further purification. LRMS (ESI): (calc) 248.1; (found) 271.1 (M+Na)⁺.

### Step 2: 2-(4-fluorobenzyloxy)-N-(3-(4-(2-hydroxyethyl)phenyl)prop-2-ynyl)acetamide (193a)

Following the same procedure as described for compound 182a (scheme 25, example 84a) but substituting 192a for 181 and 189 for methyl 3-(2-oxo-2-(prop-2-ynylamino)ethylthio)propanoate to afford 193a (578 mg, 70%) as a light orange oil. LRMS (ESI): (calc) 341.4; (found) 342.2 (MH)⁺.

### Step 3: 2-(4-fluorobenzyloxy)-N-(3-(4-(2-hydroxyethyl)phenyl)propyl)acetamide (194a)

Following the same procedure as described for compound 74 (scheme 5, example 55) but substituting 193a for 73 to afford 194a (500 mg, 86%) as a light orange oil. LRMS (ESI): (calc) 345.4; (found) 346.2 (MH)⁺.

### Step 4: 2-(4-fluorobenzyloxy)-N-(3-(4-(2-oxoethyl)phenyl)propyl)acetamide (195a)

To a stirred solution of 2-(4-fluorobenzyloxy)-N-(3-(4-(2-hydroxyethyl)phenyl)propyl)acetamide 194a (500 mg, 1.45 mmol) in dichloromethane (5 mL) at room temperature was added Dess-Martin reagent (737 mg, 1.74 mmol). The resulting solution was stirred for 2 hours prior to dilution with brine, adjustment to pH=13 with NaOH, and extraction with ethy lacetate. The combined organic extracts were dried (Na₂SO₄), filtered, and concentrated to afford 195a (392 mg, 79%) as a light yellow oil which was used in the subsequent reaction without further purification. LRMS (ESI): (calc) 343.4; (found) 344.2 (MH)⁺.

### Step 5: N-(3-(4-(2-(2-(1H-indol-3-yl)ethylamino)ethyl)phenyl)propyl)-2-(4-fluorobenzyloxy)acetamide (196a)

To a stirred solution of 2-(4-fluorobenzyloxy)-N-(3-(4-(2-oxoethyl)phenyl)propyl)acetamide 195a (196 mg, 0.571 mmol) in solvent (tetrahydrofuran or methanol) (4 mL) at room temperature was added tryptamine (101 mg, 0.628 mmol), and the resulting solution stirred for 16 hours. Reductant (NaBH(OAc)₃ or NaBH₄) (0.742 mmol) was then added, and the solution stirred for a further 16 hours prior to dilution with brine, adjustment to pH=13 with NaOH, and extraction with ethyl acetate. The combined organic extracts were dried (Na₂SO₄), filtered, and evaporated. The residue was purified by silica gel column chromatography using MeOH (0-30%) in EtOAc to afford 196a (12 mg, 4%) as a light yellow oil. (MeOD-d4) δ (ppm) ¹H: 7.58-7.53 (m,1H), 7.46-7.40 (m,2H), 7.37-7.33 (m,1H), 7.14-6.95 (m,9H), 4.59 (s,2H), 3.96 (s,2H), 3.26 (t, J=7.0 Hz, 2H), 3.00-2.92 (m,4H), 2.85 (t, J=7.0 Hz, 2H), 2.73 (t, J=7.2 Hz, 2H), 2.58 (t, J=8.0 Hz, 2H), 1.86-1.76 (m,2H). LRMS (ESI): (calc) 487.6; (found) 488.6 (MH)⁺.

### EXAMPLE 87b

### (S)-2-(4-fluorobenzyloxy)-N-(3-(4-((1-hydroxy-3-(1H-indol-3-yl)propan-2-ylamino)methyl)phenyl)propyl)acetamide (196b)

### Step 1: (S)-2-(4-fluorobenzyloxy)-N-(3-(4-((1-hydroxy-3-(1H-indol-3-yl)propan-2-ylamino)methyl)phenyl)propyl)acetamide (196b)

Following the same procedure as described for compound 196a (scheme 27, steps 2-5, example 87a), but substituting (4-iodophenyl)methanol for 192a in Step 1, and (S)-2-amino-3-(1H-indol-3-yl)propan-1-ol for tryptamine in Step 5 to afford 196b (231 mg, 50%) as a light yellow oil. (MeOD-d4) δ (ppm) ¹H, 7.49-7.45 (m,1H), 7.40-7.34 (m,3H), 7.14-7.03 (m,8H), 7.20-6.96 (m,1H), 4.51 (s,2H), 3.92 (s,2H), 3.78 (d, J=1.8 Hz, 2H), 3.64-3.52 (m,2H), 3.24 (t, J=7.2 Hz, 2H), 3.10-3.00 (m,1H), 2.91 (d, J=7.0 Hz, 2H), 2.57 (t, J=7.8 Hz, 2H), 1.84-1.74 (m,2H). LRMS (ESI): (calc) 503.6; (found) 504.9 (MH)⁺.

### EXAMPLE 88b

### (S)-2-(4-fluorobenzyloxy)-N-(3-(4-(((1-hydroxy-3-(1H-indol-3-yl)propan-2-yl)(2-hydroxyethyl)amino)methyl)phenyl)propyl)acetamide (197b)

### Step 1: (S)-2-(4-fluorobenzyloxy)-N-(3-(4-(((1-hydroxy-3-(1H-indol-3-yl)propan-2-yl)(2-hydroxyethyl)amino)methyl)phenyl)propyl)acetamide (197b)

To a stirred solution of (S)-2-(4-fluorobenzyloxy)-N-(3-(4-((1-hydroxy-3-(1H-indol-3-yl)propan-2-ylamino)methyl)phenyl)propyl)acetamide 196b (170 mg, 0.338 mmol) in acetonitrile (4 mL) at room temperature was added 2-bromoethanol (0.24 mL, 3.38 mmol) and K₂CO₃ (140 mg, 1.01 mmol). The resulting solution was heated to 60° C. for 16 hours prior to cooling, dilution with brine, adjustment to pH=10 with NaOH, and extraction with ethyl acetate. The combined organic extracts were dried (Na₂SO₄), filtered, and evaporated. The residue was purified by silica gel column chromatography using MeOH (0-30%) in EtOAc to afford 197b (67 mg, 36%) as a colorless oil. (MeOD-d4) δ (ppm) ¹H: 7.46-7.32 (m,4H), 7.27-7.21 (m,2H), 7.14-7.06 (m,5H), 7.02-6.94 (m,2H), 4.55 (s,2H), 3.94 (s,2H), 3.88 (d, J=13.7 Hz, 1H), 3.73 (d, J=13.7 Hz, 1H), 3.63-3.41 (m,4H), 3.26 (t, J=7.2 Hz, 2H), 3.25-3.16 (m,1H), 3.06 (dd, J=14.3,5.3 Hz, 1H), 2.99-2.90 (m,1H), 2.78-2.63 (m,2H), 2.61 (t, J=7.8 Hz, 2H), 1.87-1.77 (m,2H). LRMS (ESI): (calc) 547.7; (found) 548.2 (MH)⁺.

### EXAMPLE 87c-l AND 88c-o

Example 87c-l and 88c-o describe the preparation of compound 196c-l and 197c-o using the same procedures as described for compound 196a,b and 197b in Example 87a,b and 88b. Characterization data are presented in a Table 16.

TABLE 16

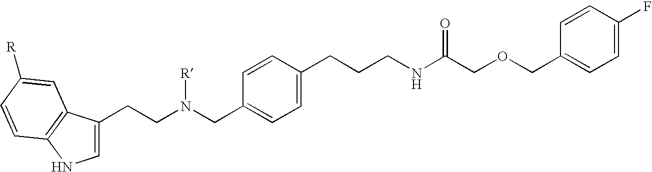

| | | | | | | Scheme |
|---|---|---|---|---|---|---|
| Ex | Cpd | R or structure | R′ or structure | Name | Characterization | (steps) |
| 87c | 196c | OMe | H | 2-(4- | (MeOD-d4)) □(ppm): 7.35 | 27(2-5) |
| | | | | fluorobenzyloxy | (dd, J = 8.6, 5.5 Hz, 2H), | |
| | | | | )-N-(3-(4-((2-(5- | 7.20 (d, J = 8.8 Hz, 1H), 7.12 | |
| | | | | methoxy-1H- | −6.95 (m, 8H), 6.73 (dd, | |
| | | | | indol-3- | J = 8.6, 2.4 Hz, 1H), 4.49 (s, | |
| | | | | yl)ethylamino) | 2H), 3.89 (s, 2H), 3.74 (s, | |
| | | | | methyl)phenyl) | 3H), 3.67 (s, 2H), 3.21 (t, | |
| | | | | propyl)acetamide | J=7.0 Hz, 2H), 2.92 - 2.81 | |
| | | | | | (m, 4H), 2.55 (t, J = 7.6 Hz, | |
| | | | | | 2H), 1.78 (quintet, J = 7.4 | |
| | | | | | Hz, 2H). | |
| | | | | | LRMS (ESI): (calc) 503.2; | |
| | | | | | (found) 504.3 (MH)⁺. | |
| 87d | 196d | OBn | H | N-(3-(4-((2-(5- | (MeOD-d4)) □(ppm): 7.43- | 27 (2-5) |
| | | | | (benzyloxy)- | 7.40 (m, 2H), 7.37-7.20 | |
| | | | | 1H-indol-3- | (m, 6H), 7.13-6.99 (m, | |
| | | | | yl)ethylamino) | 8H), 6.82 (dd, J = 8.6, 2.3 | |
| | | | | methyl)phenyl) | Hz, 1H), 5.00 (s, 2H), 4.50 | |
| | | | | propyl)-2-(4- | (s, 2H), 3.88 (s, 2H), 3.68 | |
| | | | | fluorobenzyloxy | (s, 2H), 3.18 (t, J = 7.2 Hz, | |
| | | | | )acetamide | 2H), 2.92-2.81 (m, 4H), | |
| | | | | | 2.53 (t, J = 7.4 Hz, 2H), 1.74 | |
| | | | | | (quintet, J = 7.2 Hz, 2H). | |
| | | | | | LRMS (ESI): (calc) 579.3; | |
| | | | | | (found) 580.6 (MH)⁻. | |
| 87e | 196e | F | H | N-(3-(4-((2-(5- | (MeOD-d4)) □(ppm): 7.40 | 27(2-5) |
| | | | | fluoro-1H-indol- | (dd, J = 8.6, 5.5 Hz, 2H), | |
| | | | | 3- | 7.28 (dd, J = 8.8, 4.5 Hz, | |
| | | | | yl)ethylamino) | 1H), 7.20-7.04 (m, 8H), | |
| | | | | methyl)phenyl) | 6.84 (td, J = 9.0, 2.6 Hz, 1H), | |
| | | | | propyl)-2-(4- | 4.55 (s, 2H), 3.92 (s, 2H), | |
| | | | | fluorobenzyloxy | 3.77 (s, 2H), 3.24 (t, J = 7.2 | |
| | | | | )acetamide | Hz, 2H), 2.96-2.87 (m, | |
| | | | | | 4H), 2.61 (t, J = 7.4 Hz, 2H), | |
| | | | | | 1.81 (quintet, J = 7.2 Hz, | |
| | | | | | 2H). | |
| | | | | | LRMS (ESI): (calc) 491.3; | |
| | | | | | (found) 492.7 (MH)⁻. | |
| 87f | 196f | H | H | N-(3-(4-((2- | (MeOD-d4)) □(ppm): 7.49 | 27(2-5) |
| | | | | (1H-indol-3- | (dt, J = 7.8, 1.0 Hz, 1H), 7.40 | |
| | | | | yl)ethylamino) | (dd, J = 8.8, 5.3 Hz, 2H), | |
| | | | | methyl)phenyl) | 7.32 (dt, J = 8.0, 0.8 Hz, 1H), | |
| | | | | propyl)-2-(4- | 7.17-7.02 (m, 8H), 6.98- | |
| | | | | fluorobenzyloxy | 6.94 (m, 1H), 4.54 (s, 2H), | |
| | | | | )acetamide | 3.91 (s, 2H), 3.72 (s, 2H), | |
| | | | | | 3.23 (t, J = 7.0 Hz, 2H), 2.99- | |
| | | | | | 2.87 (m, 4H), 2.60 (t, | |
| | | | | | J = 7.4 Hz, 2H), 1.80 | |
| | | | | | (quintet, J = 7.2 Hz, 2H). | |
| | | | | | LRMS (ESI): (calc) 473.3; | |
| | | | | | (found) 474.3 (MH)⁻. | |
| 88c | 197c | OMe | —CH₂CH₂OH | 2-(4- | (MeOD-d4)) □(ppm): 7.38 | 27 (2-6) |
| | | | | fluorobenzyloxy | (dd, J = 8.8, 5.5 Hz, 2H), | |
| | | | | )-N-(3-(4-(((2- | 7.24 (d, J = 8.2 Hz, 2H), 7.18 | |
| | | | | hydroxyethyl)(2 | (d, J = 8.6 Hz, 2H), 7.12- | |
| | | | | -(5-methoxy- | 7.04 (m, 4H), 6.95 (s, 1H), | |
| | | | | 1H-indol-3- | 6.84 (d, J = 2.4 Hz, 1H), 6.70 | |
| | | | | yl)ethyl)amino) | (dd, J = 8.8, 2.3 Hz, 1H), | |
| | | | | methyl)phenyl) | 4.53 (s, 2H), 3.91 (s, 2H), | |
| | | | | propyl)acetamide | 3.76-3.62 (m, 7H), 3.24 (t, | |
| | | | | | J = 7.2 Hz, 2H), 2.91-2.72 | |
| | | | | | (m, 6H), 2.59 (t, J = 7.4 Hz, | |
| | | | | | 2H), 1.80 (quintet, J = 7.2 | |
| | | | | | Hz, 2H). | |
| | | | | | LRMS (ESI): (calc) 547.3; | |
| | | | | | (found) 548.5 (MH)⁻. | |
| 88d | 197d | OBn | —CH₂CH₂OH | N-(3-(4-(((2-(5- | (MeOD-d4)) □(ppm): 7.43- | 27 (2-6) |
| | | | | (benzyloxy)- | 7.18 (m, 10H), 7.09-7.02 | |
| | | | | 1H-indol-3- | (m, 4H), 6.95-6.92 (m, | |
| | | | | yl)ethyl)(2- | 2H), 6.78 (dd, J = 8.8, 2.3 | |
| | | | | hydroxyethyl) | Hz, 1H), 4.96 (s, 2H), 4.50 | |
| | | | | amino)methyl) | (s, 2H), 3.87 (s, 2H), 3.66- | |
| | | | | phenyl)propyl)-2- | 3.60 (m, 4H), 3.17 (t, J = 7.0 | |
| | | | | (4- | Hz, 2H), 2.88-2.84 (m, | |
| | | | | fluorobenzyloxy | 2H), 2.76-2.68 (m, 4H), | |
| | | | | )acetamide | 2.51 (t, J = 7.4 Hz, 2H), 1.73 | |
| | | | | | (quintet, J = 7.6 Hz, 2H). | |
| | | | | | LRMS (ESI): (calc) 523.3; | |
| | | | | | (found) 524.3 (MH)⁻. | |
| 88e | 197e | F | —CH₂CH₂OH | N-(3-(4-(((2-(5- | (MeOD-d4)) □(ppm): 7.39 | 27 (2-6) |
| | | | | fluoro-1H-indol- | (dd, J = 8.8, 5.5 Hz, 2H), | |
| | | | | 3-yl)ethyl)(2- | 7.25-7.21 (m, 3H), 7.15- | |
| | | | | hydroxyethyl) | 7.03 (m, 5H), 6.97 (dd, | |
| | | | | amino)methyl) | J = 10.0, 2.3 Hz, 1H), 6.79 | |
| | | | | phenyl)propyl)-2- | (td, J = 9.0, 2.3 Hz, 1H), 4.54 | |
| | | | | (4- | (s, 2H), 3.91 (s, 2H), 3.69 | |
| | | | | fluorobenzyloxy | (s, 2H), 3.63 (t, J = 6.5 Hz, | |
| | | | | )acetamide | 2H), 3.25 (t, J = 7.0 Hz, 2H), | |
| | | | | | 2.88-2.71 (m, 8H), 2.61 (t, | |
| | | | | | J = 7.7 Hz, 2H), 1.82 | |
| | | | | | (quintet, J = 7.4 Hz, 2H). | |
| | | | | | LRMS (ESI): (calc) 535.3; | |
| | | | | | (found) 536.6 (MH)⁻. | |
| 88g | 197g | H | 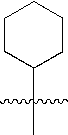 | N-(3-(4-(((2- 1H-indol-3- yl)ethyl)(cyclohexyl) amino)methyl) phenyl)propyl)- 2-(4- fluorobenzyloxy )acetamide | (MeOD-d4)) □(ppm): 8.55 (s, 1H), 7.41-7.37 (m, 4H), 7.33-7.26 (m, 3H), 7.10-7.03 (m, 4H), 6.96 (t, J = 7.8 Hz, 1H), | |
| # 4.55 (s, 2H), 4.24 (s, 2H), 3.93 (s, 2H), 3.28-3.24 (m, SH), 2.99 (t, J = 7.4 Hz, 2H), 2.67 (t, J = 7.6 Hz, 2H), 2.08 (d, J = 10.7 Hz, 2H), 1.92-1.80 (m, 4H), 1.70-1.53 (m, 3H), 1.37-1.16 (m, 3H). LRMS (ESI): (calc) 555.3; (found) 556.3 (MH)⁻. | 27 (2-6) | | | | | |
| 88h | 197h | H | 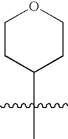 | N-(3-(4-(((2- (1H-indol-3- yl)ethyl)(tetrahydro -2H-pyran-4- yl)amino)methyl) phenyl)propyl) -2-(-4- fluorobenzyloxy )acetamide | (MeOD-d4)) □(ppm): 7.40-7.21 (m, 8H), 7.08-6.99 (m, 4H), 6.94-6.90 (m, 1H), 4.54 (s, 2H), 4.02-3.92 (m, 6H), 3.37 (t, J = 10.4 Hz, 2H), 3.26 (t, J = 7.2 Hz, 2H), | |
| # 3.18-3.17 (m, 1H), 3.03-2.96 (m, 2H), 2.91-2.87 (m, 2H), )acetamide 2.65 (t, J = 7.2 Hz, 2H), 1.88-1.69 (m, 6H). LRMS (ESI): (calc) 557.3; (found) 558.3 (MH)⁺. | 27 (2-6) | | | | | |
| 88k | 197k | H | —CH₂CH₂CH₂OH | N-(3-(4-(((2- | (MeOD-d4) □(ppm): 7.39- | 27 (2-6) |
| | | | | (1H-indol-3- | 7.32 (m, 3H), 7.29 (d, J = 8.0 | |
| | | | | yl)ethyl)(3- | Hz, 1H), 7.21 (d, J = 8.0 Hz, | |
| | | | | hydroxypropyl) | 2H), 7.13 (d, J = 7.8 Hz, 2H), | |
| | | | | amino)methyl) | 7.08-7.01 (m, 3H), 6.96 (s, | |
| | | | | henyl)propyl)- | 1H), 6.92 (t, J = 7.6 Hz, 1H), | |
| | | | | 2-(4- | 4.52 (s, 2H), 3.90 (s, 2H), | |
| | | | | fluorobenzyloxy | 3.64-3.60 (m, 4H), 3.24 (t, | |
| | | | | )acetamide | J = 7.2 Hz, 2H), 2.93-2.89 | |
| | | | | | (m, 2H), 2.76-2.68 (m, | |
| | | | | | 4H), 2.60 (t, J = 7.4 Hz, 2H), | |
| | | | | | 1.85-1.79 (m, 4H). | |
| | | | | | LRMS (ESI): (calc) 531.3; | |
| | | | | | (found) 532.4 (MH)⁺. | |
| 88l | 197l | H | CH₂CH₂(OH)CH₂OH | N-(3-(4-(((2- | (MeOD-d4) ═(ppm): 7.39- | 27 (2-6) |
| | | | | (1H-indol-3- | 7.32 (m, 3H), 7.29 (d, J = 8.2 | |
| | | | | yl)ethyl)(2,3- | Hz, 1H), 7.22 (d, J = 8.0 Hz, | |
| | | | | dihydroxypropy | 2H), 7.12-7.01 (m, 5H), | |
| | | | | l)amino)methyl) | 6.95 (s, 1H), 6.91 (t, J = 7.0 | |
| | | | | phenyl)propyl)- | Hz, 1H), 4.52 (s, 2H), 3.92 | |
| | | | | 2-(4- | (s, 2H), 3.80-3.65 (m, 3H), | |
| | | | | fluorobenzyloxy | 3.60-3.54 (m, 1H), 3.48- | |
| | | | | )acetamide | 3.43 (m, 1H), 3.24 (t, J = 7.0 | |
| | | | | | Hz, 2H), 2.94-2.89 (m, | |
| | | | | | 2H), 2.81-2.77 (m, 2H), | |
| | | | | | 2.70-2.57 (m, 4H), 1.81 | |
| | | | | | (quintet, J = 7.4 Hz, 2H). | |
| | | | | | LRMS (ESI): (calc) 547.3; | |
| | | | | | (found) 548.3 (MH)⁺. | |

| | | | | |
|---|---|---|---|---|
| 87g | 196g | 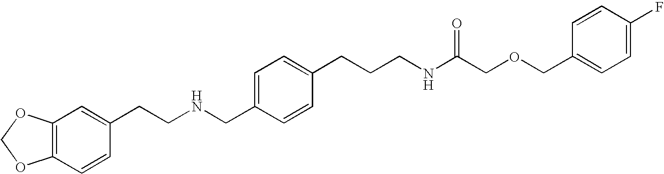 | N-(3-(4-((2- (benzo[d][1,3]dioxol-5- yl)ethylamino) methyl)phenyl) propyl)-2-(4- fluorobenzyloxy )acetamide | (MeOD-d4)) □(ppm): 7.40 (dd, J = 8.4, 5.9 Hz, 2H), 7.19-7.13 (m, 4H), 7.07 (t, J = 8.8 Hz, 2H), 6.71-6.60 (m, 3H), 5.86 (s, 2H), 4.54 (s, 2H), 3.91 (s, 2H), |
| # 3.69 (s, 2H), 3.23 (t, J = 7.0 Hz, 2H), 2.77-2.68 (m, 4H), 2.59 (t, J = 7.4 Hz, 2H), 1.80 (quintet, J = 7.4 Hz, 2H). LRMS (ESI): (calc) 478.2; (found) 479.6 (MH)⁺. | 27(2-5) | | | |
| 87h | 196h | 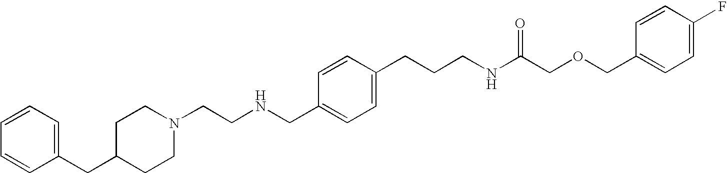 | N-(3-(4-((2-(4- benzylpiperidin -1- yl)ethylamino) methyl)phenyl) propyl)-2-(4- fluorobenzyloxy )acetamide | (MeOD-d4)) □(ppm): 7.41 (dd, J = 8.2, 5.5 Hz, 2H), 7.26-7.22 (m, 4H), 7.18-7.06 (m, 7H), 4.57 (s, 2H), 3.92 (s, 2H), 3.72 (s, 2H), 3.24 (t, J = 7.0 Hz, 2H), 2.84 (d, J = |
| # 11.8 Hz, 2H), 2.69 (t, J = 6.6 Hz, 2H), 2.61 (t, J = 7.4 Hz, 2H), 2.53-2.45 (m, 4H), 1.96-1.90 (m, 2H), 1.81 (quintet, J = 7.2 Hz, 2H), 1.62-1.51 (m, 3H), 1.31-1.21 (m, 2H). LRMS (ESI): (calc) 531.3; (found) 532.5 (MH)⁺. | 27(2-5) | | | |
| 88i | 197i | 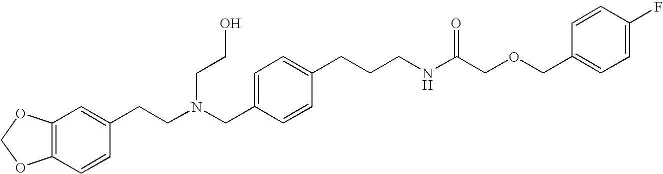 | N-(3-(4-(((2- (benzo[d][1,3]dioxol-5- yl)ethyl)(2- hydroxyethyl) amino)methyl) phenyl)propyl)-2- (4- fluorobenzyloxy )acetamide | (MeOD-d4) □(ppm): 7.39 (dd, J = 8.8, 5.5 Hz, 2H), 7.20 (d, J = 8.0 Hz, 2H), 7.12 (d, J = 8.2 Hz, 2H), 7.07 (t, J = 8.8 Hz, 2H), 6.67 (d, J = 7.9 |
| # Hz, 1 H), 6.60-6.55 (m, 2H), 5.85 (s, 2H), 4.55 (s, 2H), 3.92 (s, 2H), 3.64 (s, 2H), 3.24 (t, J = 7.0 Hz, 2H), 2.67-2.58 (m, 8H), 1.81 (quintet, J = 7.4 Hz, 2H). LRMS (ESI): (calc) 522.3; (found) 523.3 (MH)⁺. | 27 (2-6) | | | |
| 88j | 197j | 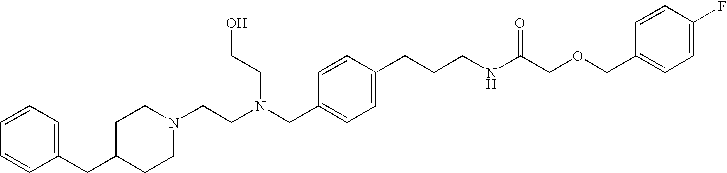 | N-(3-(4-(((2-(4- benzylpiperidin -1-yl)ethyl)(2- hydroxyethyl) amino)methyl) phenyl)propyl)-2- (4-fluorobenzyloxy )acetainide | (MeOD-d4) □(ppm): 7.40 (dd, J = 8.5, 5.5 Hz, 2H), 7.26-7.22 (m, 4H), 7.16-7.05 (m, 7H), 4.55 (s, 2H), 3.92 (s, 2H), 3.60-3.56 (m, 4H), 3.22 (t, J = 7.0 Hz, 2H), 2.77 |
| # (d, J = 11.7 Hz, 2H), 2.66-2.57 (m, 6H), 2.53-2.42 (m, 4H), 1.96-1.89 (m, 2H), 1.78 (quintet, J = 7.2 Hz, 2H), 1.60-1.48 (m, 3H), 1.29-1.20 (m, 2H). LRMS (ESI): (calc) 575.4; (found) 576.6 (MH)⁺. | 27 (2-6) | | | |
| 87i | 196i | 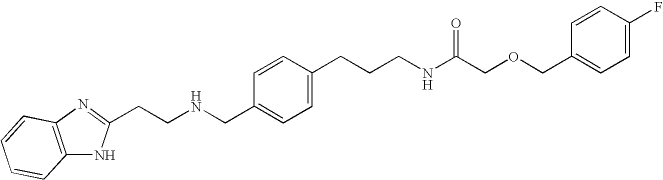 | N-(3-(4-((2- (1H- benzo[d]imidazol-2- yl)ethylainino) methyl)phenyl) propyl)-2-(4- fluorobenzyloxy )acetamide | (MeOD-d4) □(ppm): 7.50-7.47 (m, 2H), 7.38 (dd, J = 8.4, 5.5 Hz, 2H), 7.24 (d, J = 7.8 Hz, 2H), 7.20-7.17 (m, 2H), 7.14 (d, J = 8.0 Hz, 2H), 7.06 (t, J= 8.8 Hz, 2H), 4.54 |
| # (s, 2H), 3.91 (s, 2H), 3.80 (s, 2H), 3.23 (t, J = 7.0 Hz, 2H), 3.09 (s, 4H), 2.59 (t, J = 7.4 Hz, 2H), 1.79 (quintet, J = 7.4 Hz, 2H). LRMS (ESI): (calc) 474.2; found 475.3 (MH)⁺. | 27 (2-5) | | | |
| 88m | 197m | 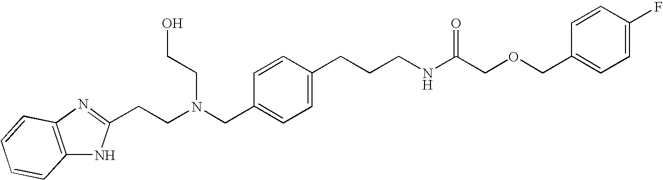 | N-(3-(4-(((2- (1H- benzo[d]imidazol -2-yl)ehtyl)(2- hydroxoyethyl) amino)methyl) phenyl)propyl)-2- (4- fluorobenzyloxy )acetamide | (MeOD-d4) □(ppm): 7.47-7.45 (m, 2H), 7.40 (dd, J = 8.8, 5.7 Hz, 2H), 7.20-7.15 (m, 2H), 7.09-7.02 (m, 4H), 6.90 (d, J = 8.2 Hz, 2H), 3.67 (t, |
| # J = 5.6 Hz, 2H), 2H), 4.55 (s, 2H), 3.92 (s, 3.62 (s, 2H), 3.19 (t, J = 7.2 Hz, 2H), 3.01 (t, J = 6.8 Hz, 2H), 2.90 (t, J = 6.1 Hz, 2H), 2.72 (t, J = 6.7 Hz, 2H), 2.50 (t, J = 7.4 Hz, 2H), 1.72 (quintet, J = 7.2 Hz, 2H). LRMS (ESI): (calc) 518.3; (found) 519.4 (MH)⁺. | 27 (2-6) | | | |
| 88n | 197n | 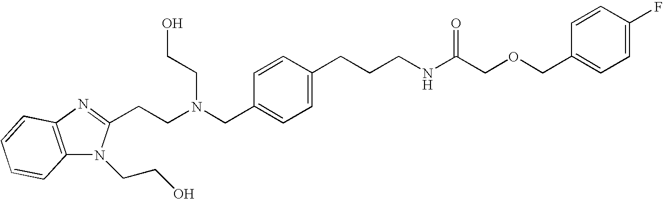 | 2-(4- fluorobenzyloxy )-N-(3-(4-(((2- hydroxyethyl)(2 -1(-2- hydroxyethyl)- 1H- benzo[d]imidazol -2- yl)ethyl)amino) methyl)phenyl) propyl)acetanide | (MeOD-d4) □(ppm): 7.56 7.54 (m, 1 H), 7.44-7.38 (m, 3H), 7.25-7.18 (m, 2H), 7.10-7.05 (m, 4H), 6.95 (d, J = 8.0 Hz, 2H), 4.56 (s, |
| # 2H), 4.17 (t, J = 5.5 Hz, 2H), 3.92 (s, 2H), 3.77 (t, J = 5.3 Hz, 2H), 3.68-3.62 (m, 4H), 3.23 (t, J = 7.2 Hz, 2H), 3.12 (t, J = 7.0 Hz, 2H), 2.98 (t, J = 7.2 Hz, 2H), 2.75 (t, J = 5.7 Hz, 2H), 2.54 (t, J = 7.6 Hz, 2H), 1.77 (quintet, J = 7.6 Hz, 2H). LRMS (ESI): (calc) 562.3; (found) 563.4 | | | | |
| # (MH)⁺. | 27 (2-5) | | | |
| 87k | 196k | 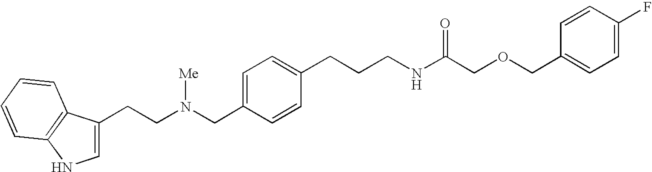 | N-(3-(4-(((2- (1H-indol-3- yl)ethyl)(methyl )amino)methyl) phenyl)propyl)- 2-(4- fluorobenzyloxy )acetamide | (MeOD-d4) d(ppm) ¹H 7.46-7.38 (m, 3H), 7.35-7.32 (m, 1 H), 7.29-7.25 (m, 2H), 7.22-7.17 (m,2H), 7.1-7.06 (m, 3H), 7.03 (s,1H), 7.01-6.95 (m, 1H), 4.57 (s, 2H), 3.94 (s, 2H), 3.61 |
| # (s,2H), 3.28 (t, J = 7.2 Hz, 2H), 3.28 (t, J = 7.2 Hz, 2H), 3.03-2.96 (m, 2H), 2.77-2.70 (m, 2H), 2.65 (t, J = 8.0 Hz, 2H), 2.35 (s, 3H), 1.90-1.80 (m,2H) LRMS (ESI): (calc) 487.6 (found) 488.3 (MH)⁺. | 27 (2-5) | | | |
| 87l | 196l | 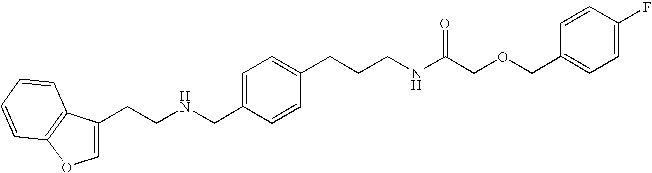 | N-(3-(4-((2- (benzofuran-3- yl)ethylamino) methyl)phenyl) propyl)-2-(4- fluorobenzyloxy )acetainide | (MeOD-d4) □(ppm): 7.54-7.51 (m, 2H), 7.43-7.37 (m, 3H), 7.28-7.04 (m, 8H), 4.55 (s, 2H), 3.91 (s, 2H), 3.74 (s, 2H), 3.24 (t, J = 7.0 Hz, 2H), 2.89 (s, 4H), 2.60 (t, J = 7.4 Hz, 2H), 1.80 (quintet, |
| # J = 7.2 Hz, 2H). LRMS (ESI): (calc) 474.2; (found) 475.5 (MH)⁺. | 27 (2-5) | | | |
| 88o | 197o | 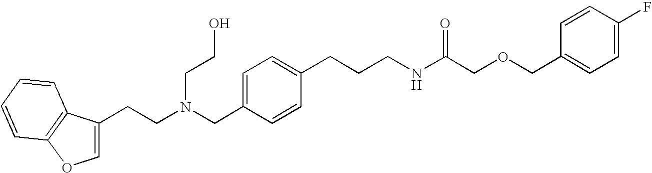 | N-(3-(4-(((2- (benzofuran-3- yl)ethyl)(2- hydroxyethyl) amino)methyl) phenyl)propyl)-2- (4- fluorobenzyloxy )acetamide | (MeOD-d4) d(ppm): 7.49 (s, 1H), 7.41-7.36 (m, 4H), 7.24-7.19 (m, 3H), 7.15-7.04 (m, 5H), 4.55 (s, 2H), 3.92 (s, 2H), 3.68 (s, 2H), 3.62 (t, J = 6.5 Hz, 2H), 3.24 (t, J = 7.2 Hz, 2H), |
| # 2.86-2.78 (m, 4H), 2.72 (t, J = 6.3 Hz, 2H), 2.60 (t, J = 7.4 Hz, 2H), 1.81 (quintet, J = 7.4 Hz, 2H). LRMS (ESI): (calc) 518.3; found 519.4 (MH)⁺. | 27 (2-6) | | | |

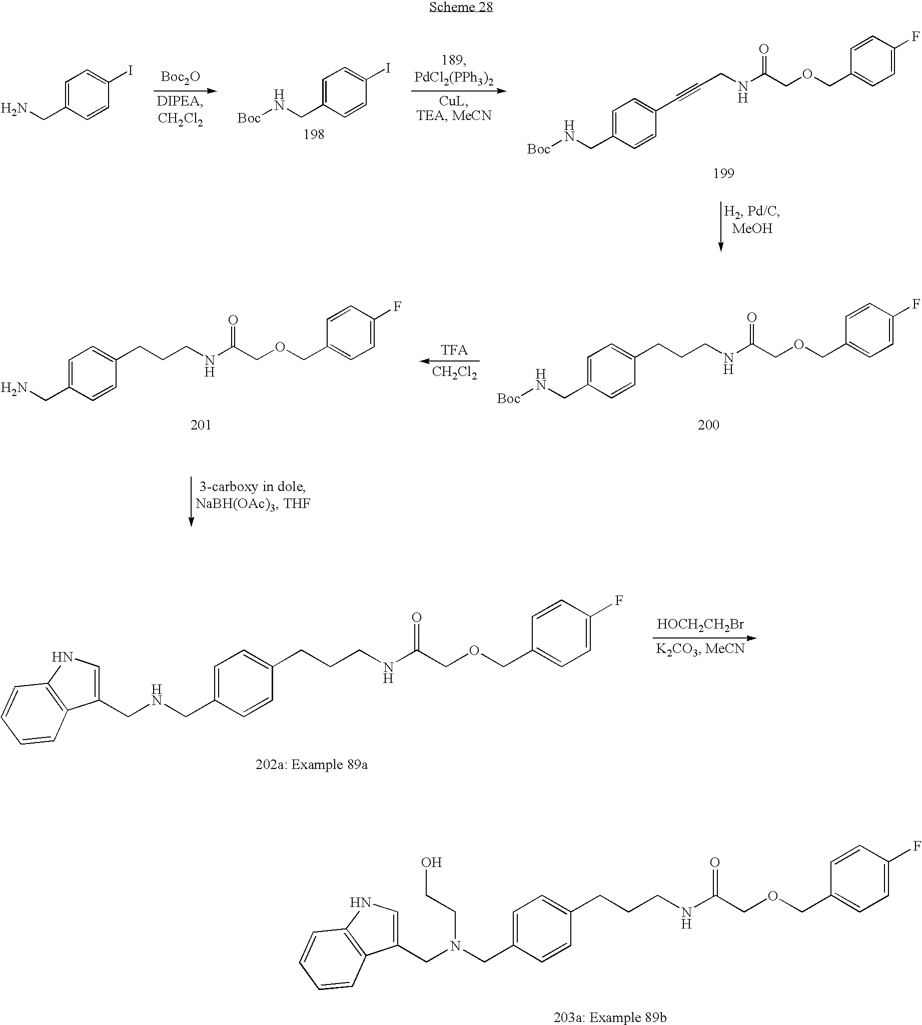

### EXAMPLE 89a

### N-(3-(4-(((1H-indol-3-yl)methylamino)methyl)phenyl)propyl)-2-(4-fluorobenzyloxy)acetamide (202a)

### Step 1: tert-butyl 4-iodobenzylcarbamate (198)

To a stirred solution of (4-iodophenyl)methanamine hydrochloride (941 mg, 3.49 mmol) in dichloromethane (10 mL) at room temperature was added diisopropylethylamine (0.73 mL, 4.19 mmol) and di-tert-butyl dicarbonate (914 mg, 4.19 mmol). The resulting solution was stirred for 2 hours prior to dilution with brine, adjustment to pH=1 with HCl, and extraction with ethyl acetate. The combined organic extracts were dried (Na₂SO₄), filtered, and concentrated. The residue was the taken up in ethyl acetate, and triturated with hexanes to afford 198 (600 mg, 52%) as a white solid. LRMS (ESI): (calc) 333.2; (found) 334.2 (MH)⁺.

### Step 2: tert-butyl 4-(3-(2-(4-fluorobenzyloxy)acetamido)prop-1-ynyl)benzylcarbamate (199)

Following the same procedure as described for compound 182a (scheme 25, example 84a) but substituting 198 for 181 and 189 for methyl 3-(2-oxo-2-(prop-2-ynylamino)ethylthio)propanoate to afford 199 (553 mg, 82%) as a light yellow solid. LRMS (ESI): (calc) 426.5; (found) 427.2 (MH)⁺.

### Step 3: tert-butyl 4-(3-(2-(4-fluorobenzyloxy)acetamido)propyl)benzylcarbamate (200)

Following the same procedure as described for compound 74 (scheme 5, example 55) but substituting 199 for 73 to afford 200 (505 mg, 90%) as a white solid. LRMS (ESI): (calc) 430.5; (found) 431.0 (MH)⁺.

### Step 4: N-(3-(4-(aminomethyl)phenyl)propyl)-2-(4-fluorobenzyloxy)acetamide (201)

Following the same procedure as described for compound 2 (step 2, scheme 1, example 1) but substituting 200 for 1 to afford 201 (378 mg, 97%) as light white solid. LRMS (ESI): (calc) 330.4; (found) 331.2 (MH)⁺.

### Step 5: N-(3-(4-(((1H-indol-3-yl)methylamino)methyl)phenyl)propyl)-2-(4-fluorobenzyloxy)acetamide (202a)

Following the same procedure as described for compound 196a (step 5, scheme 27, example 87a) but substituting 1H-indole-3-carbaldehyde for 195a and 201 for tryptamine to afford 202a (42 mg, 8%) as white solid. (MeOD-d4) δ (ppm) ¹H: 7.55 (dt, J=7.8,1.0 Hz, 1H), 7.44-7.36 (m,3H), 7.29-7.17 (m,5H), 7.15-7.01 (m,4H), 4.56 (s,2H), 3.95 (s,2H), 3.94 (s,2H), 3.78 (s,2H), 3.28 (t, J=7.3 Hz, 2H), 2.64 (t, J=8.0 Hz, 2H), 1.90-1.80 (m,2H). LRMS (ESI): (calc) 459.6; (found) 460.3 (MH)⁺.

### EXAMPLE 89b

### N-(3-(4-((((1H-indol-3-yl)methyl)(2-hydroxyethyl)amino)methyl)phenyl)propyl)-2-(4-fluorobenzyloxy)acetamide (203a)

### Step 1: N-(3-(4-((((1H-indol-3-yl)methyl)(2-hydroxyethyl)amino)methyl)phenyl)propyl)-2-(4-fluorobenzyloxy)acetamide (203a)

Following the same procedure as described for compound 197b (step 1, scheme 27, example 88b) but substituting 202a for 196b to afford 203a (5 mg, 14%) as a light yellow oil. (MeOD-d4) δ (ppm) ¹H: 7.63-7.59 (m,1H), 7.44-7.35 (m,3H), 7.32-7.27 (m,2H), 7.24-7.16 (m,3H), 7.14-7.07 (m,3H), 7.05-7.00 (m,1H), 4.56 (s,2H), 3.93 (s,2H), 3.87 (s,2H), 3.69-3.62 (m,4H), 3.28 (t, J=7.0 Hz, 2H), 2.69 (t, J=6.7 Hz, 2H), 2.65 (t, J=7.8 Hz, 2H), 1.90-1.80 (m,2H). LRMS (ESI): (calc) 503.6; (found) 504.2 (MH)⁺.

### EXAMPLE 89c-f

Example 89c-f describe the preparation of compound 202-f using the same procedures as described for compound 202a and 203a in Example 89a and 89b. Characterization data are presented in a Table 17.

TABLE 17

| | | | | | Scheme |
|---|---|---|---|---|---|
| Ex | Cpd | structure | Name | Characterization | (steps) |
| 89c | 202c | 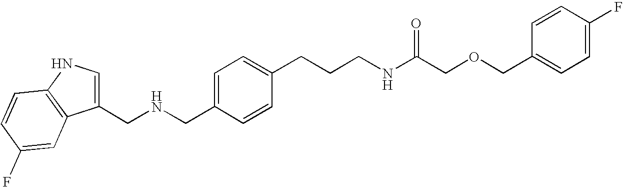 | N-(3-(4-(((5-fluoro- 1H-indol-3- yl)methylamino)methyl )phenyl)propyl)- 2-(4- fluorobenzyloxy)acetamide | (MeOD-d4) □(ppm) ¹H: 7.44-7.23 (m, 9H), 7.13-7.06 (m, 2H), 6.98-6.91 (m, 1H), 4.57 (s, 2H), 4.17 (s, 2H), 4.00 (s, 2H), 3.94 (s,2H), 3.27 (t, J = 7.0 Hz, 2H), 2.66 (t, J =7.8 Hz, | |
| # 2H), 1.90-1.80 (m, 2H) LRMS (ESI): (calc) 477.5 (found) 478.4 (MH)⁺. | 28 (1-5) | | | | |
| 89d | 203d | 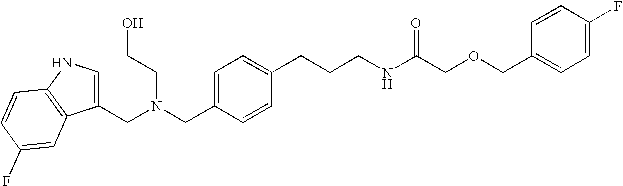 | N-(3-(4-((((5-fluoro- 1H-indol-3- yl)methyl)(2- hydroxyethyl)amino )methyl)phenyl) propyl)-2-(4- fluorobenzyloxy) acetamide | (MeOD-d4) □(ppm) ¹H: 7.44-7.38 (m, 2H), 7.33-7.22 (m, 5H), 7.21-7.16 (m, 2H), 7.13-7.07 (m, 2H), 6.91-6.84 (m, 1H), 4.56 (s, 2H), 3.93 (s, 2H), 3.79 (s, 2H), | |
| # 3.67-3.62 (m, 4H), 3.28 (t, J = 7.2 Hz, 2H), 2.70-2.61 (m, 4H), 1.90-1.80 (m, 2H) LRMS (ESI): (calc) 521.6 (found) 544.2 (M + Na)³⁰ . | 28 (1-6) | | | | |
| 89e | 203d | 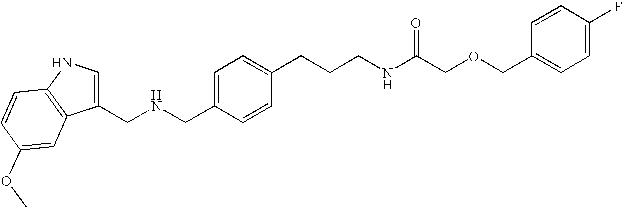 | 2-(4- fluorobenzyboxy)-N- (3-(4-(((5-methoxy- 1H-indol-3- yl)methylamino) methyl)phenyl)propyl) acetamide | (MeOD-d4) □(ppm) ¹H: 7.46-7.41 (m, 2H), 7.40-7.35 (m, 3H), 7.34-7.27 (m, 3H), 7.14-7.07 (m, 3H), 6.84 (dd, J = 8.8, 2.3 Hz, 1H), 4.59 (s, 2H), 4.26 (s, 2H), 4.09 (s, 2H), 3.96 | |
| # (s, 2H), 3.85 (s, 3H), 3.28 (t, J = 7.0 Hz, 2H), 2.68 (t, J =8.0 Hz, 2H), 1.90-1.80 (m, 2H) LRMS (ESI): (calc) 489.6 (found) 512.3 (M + Na)+ | 28 (1-5) | | | | |
| 89f | 203f | 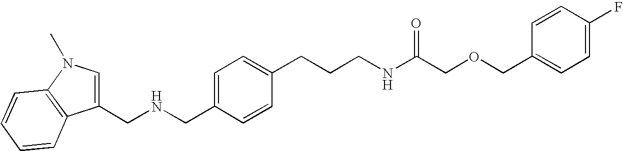 | 2-(4- fluorobenzyloxy)-N- (3-(4-(((1-methyl- 1H-indol-3- yl)methylamino)methyl )phenyl)propyl) acetamide | (MeOD-d4) □(ppm) ¹H: 7.59 (dt, J =8.0, 0.8 Hz, 1H), 7.44-7.37 (m, 3H), 7.33-7.28 (m, 2H), 7.26-7.20 (m, 4H), 7.14-7.06 (m, 3H), 4.55 (s, 2H), 4.11 (s, 2H), 3.93 (s, 4H), 3.79 | |
| # (s, 3H), 3.26 (t, J = 7.0 Hz, 2H), 2.64 (t, J = 8.0 Hz, 2H), 1.88-1.79 (m, 2H) LRMS (ESI): (calc) 473.6 (found) 474.3 (MH)+ | 28 (1-5) | | | | |

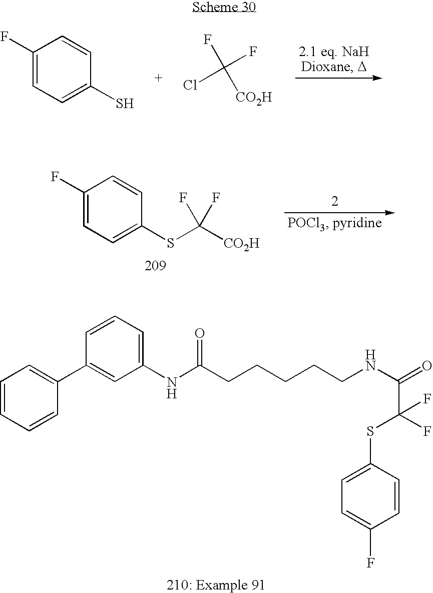

### EXAMPLE 91

### N-(biphenyl-3-yl)-6-(2,2-difluoro-2-(4-fluorophenylthio)acetamido)hexanamide (210)

### Step 1: 2,2-difluoro-2-(4-fluorophenylthio)acetic acid (209).

4-Fluorothiophenol (1.64 mL, 15.3 mmol) and 2-chloro-2,2-difluoroacetic acid (1.47 mL, 15.3 mmol) were dissolved in dioxane, cooled to 0° C., and sodium hydride (60%, 1.28 g, 32.1 mmol) was added. The mixture was then raised to room temperature and, when bubbling had ceased, was heated at 100° C. for 3 hours. The mixture was then cooled to room temperature, 5 mL of ethanol was added, and the suspension poured into a mixture of ice in 1 N HCl. The mixture was then extracted with ethyl acetate and the organic layer washed with water, then brine and dried (MgSO₄). The residue was triturated with hexanes; the solvent was removed to afford 209 as a beige solid (3.05 g, 90%). LRMS (ESI): (calc) 222.0; (found) 220.9 (M)-.

### Step 2: N-(biphenyl-3-yl)-6-(2,2-difluoro-2-(4-fluorophenylthio)acetamido)-hexanamide (210)

Amide 2 (50 mg, 0.177 mmol) and acid 209 (39 mg, 0.177 mmol) were dissolved in pyridine (2 mL) and cooled to 0° C. Phosphoryl trichoride (19 μL, 0.212 mmol) was then added dropwise and the mixture left to return to room temperature overnight. The solution was then poured into ice water, extracted with ethyl acetate, and the combined organic layers were washed with water, brine and dried (MgSO₄). The residue was purified by column chromatography (25% to 75% ethyl acetate in hexanes) to obtain compound 210 (67 mg, 78%) as a colorless oil. (CD₃OD) δ (ppm) ¹H: 7.85 (s, 1H), 7.60 (m, 4H), 7.51 (d, J=7.6 Hz, 1H), 7.32-7.42 (m, 5H), 7.16 (t, J=8.4 Hz, 2H), 3.18 (t, J=6.8 Hz, 2H), 2.4 (t, J=7.2 Hz, 2H), 1.71 (quin, J=8.0 Hz, 2H), 1.50 (quin, J=7.2 Hz, 2H), 1.34 (m, 2H). LRMS (ESI): (calc) 486.1; (found), 509.1 (M+Na)⁺.

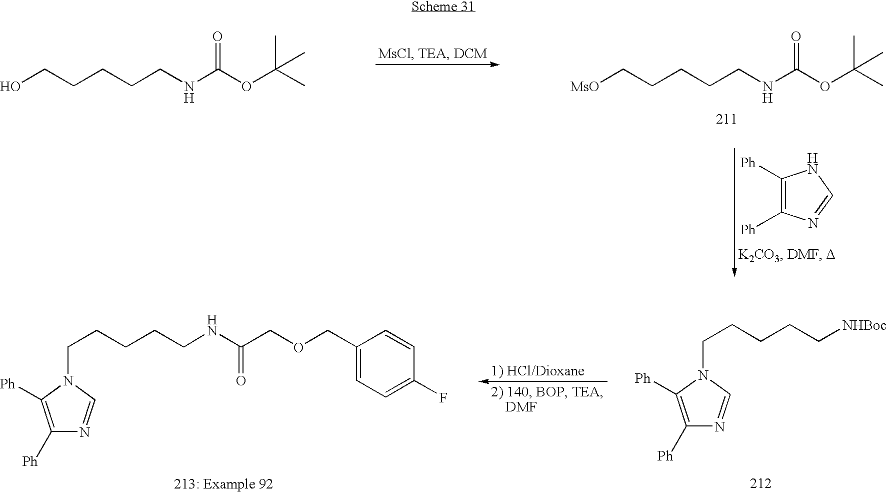

### EXAMPLE 92

### N-(5-(4,5-diphenyl-1H-imidazol-1-yl)pentyl)-2-(4-fluorobenzyloxy)acetamide (213)

### Step 1: 5-(tert-butoxycarbonylamino)pentyl methanesulfonate (211).

tert-Butyl 5-hydroxypentylcarbamate (2 g, 9.8 mmol) was dissolved in anhydrous dichloromethane (20 mL) and cooled to 0° C. Triethylamine (2.46 mL, 17.7 mmol) was added dropwise followed by slow addition of methanesulfonyl chloride (1.13 mL, 14.7 mmol). The mixture was stirred at room temperature overnight and quenched with saturated sodium bicarbonate solution. The reaction mixture was extracted with dichloromethane and the combined organic layers washed with water and brine. The organic extract was dried (MgSO₄) filtered, and concentrated to afford 211 as a yellow solid in quantitative yield. LRMS (ESI): (calc) 281.1; (found), 304.1 (M+Na)⁺.

### Step 2: tert-butyl 5-(4,5-diphenyl-1H-imidazol-1-yl)pentylcarbamate (212).

4,5-diphenyl-1H-imidazole (200 mg, 0.907 mmol) and mesylate 211 (280 mg, 1 mmol) were dissolved in DMF. Potassium carbonate (248 mg, 1.8 mmol) was then added and the mixture heated at 90° C. for 14 hours. The mixture was cooled and partitioned between water and ethyl acetate and the organic layer was washed with water, then once with brine and dried (MgSO₄). The solvent was removed and the residue purified by column chromatography (50% to 80% ethyl acetate in hexanes) to obtain compound 212 (294 mg, 81%). LRMS (ESI): (calc) 405.24; (found) 406.31 (MH)⁺.

### Step 3: N-(5-(4,5-diphenyl-1H-imidazol-1-yl)pentyl)-2-(4-fluorobenzyloxy)acetamide (213)

Compound 212 (150 mg, 0.369 mmol) was dissolved in 4 M HCl in dioxane (922 uL, 3.69 mmol) and stirred at room temperature for 1 hour. The residue was used in the next step without further purification.

Following the same procedure as described for compound 1 (scheme 1, example 1) but substituting 140 for N-Boc-caproic acid and residue amine for 3-phenyl aniline to afford 213 (144 mg, 83%) as a colorless oil. (CD₃OD) δ (ppm) ¹H: 7.80 (s, 1H), 7.45 (m, 3H), 7.31-7.35 (m, 6H), 7.13-7.16 (m, 3H), 7.05 (m, 2H), 4.51 (s, 2H), 3.88 (m, 4H), 3.11 (t, 2H, J=7.2 Hz), 1.54 (quin, 2H, J=7.2 Hz), 1.36 (quin, 2H, J=7.2 Hz), 1.14 (m, 2H). LRMS (ESI): (calc) 471.2; (found), 472.4 (MH)⁺.

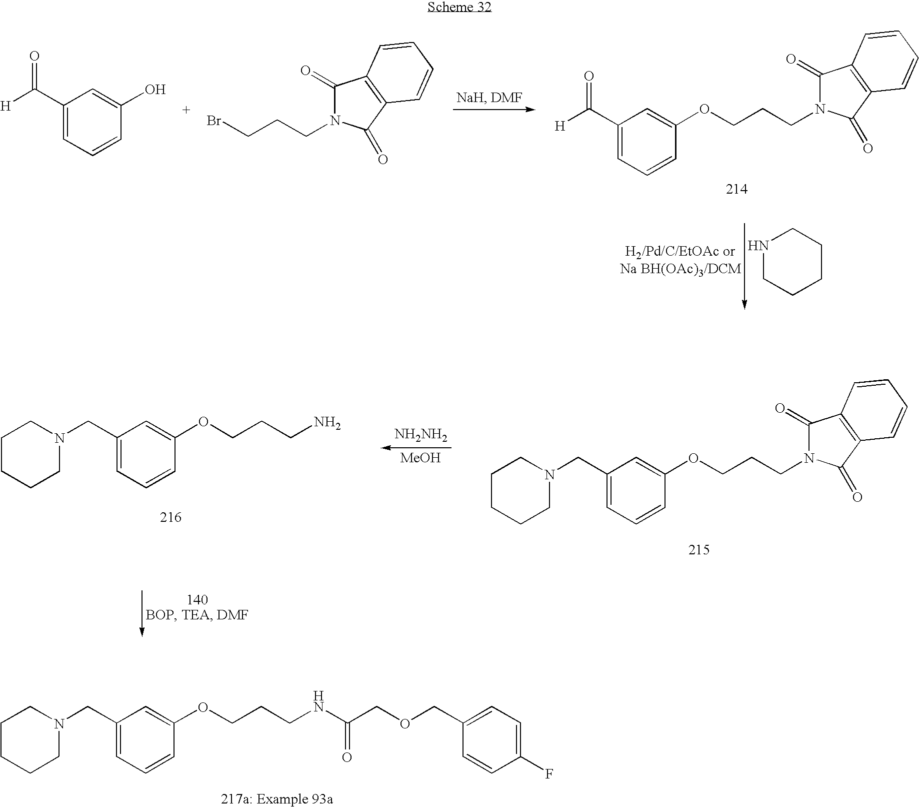

### EXAMPLE 93a

### 2-(4-fluorobenzyloxy)-N-(3-(3-(piperidin-1-ylmethyl)phenoxy)propyl)acetamide (217a)

### Step 1: 3-(3-(1,3-dioxoisoindolin-2-yl)propoxy)benzaldehyde (214)

Following the same procedure as described for compound 42 (scheme 2, step 1) but substituting 3-hydroxybenzaldehyde for (4-(methylthio)phenyl)methanol, bromopropyl phtalamide for bromomethyl acetate and DMF for THF to afford 214 (3.55 g, 70%) as a white solid.

(DMSO-d6) δ(ppm): 9.90 (s, 1H), 7.84-7.79 (m, 4H), 7.47-7.42 (m, 2H), 7.26 (d, J=2.2 Hz, 1H), 7.12-7.08 (m, 1H), 4.07 (t, J=5.9 Hz, 2H), 3.76 (t, J=6.7 Hz, 2H), 2.06 (quintet, J=6.3 Hz, 2H).

### Step 2: 2-(3-(3-(piperidin-1-ylmethyl)phenoxy)propyl)isoindoline-1,3-dione (215)

To a solution of 214 (3.55 g, 11.49 mmol) in ethyl acetate (50 mL) were added piperdine (1.14 mL) and Pd/C (500 mg). The reaction was stirred at room temperature under a H₂ atmosphere for 16 hours. The Pd/C was filtered off and the reaction mixture was concentrated. The residue was purified by silica gel column chromatography with a gradient of ethyl acetate (75-100%) in hexanes to afford 215 (2.75 g, 64%) as a light yellow oil. (DMSO-d6) δ(ppm): 7.86-7.80 (m, 4H), 7.13 (t, J=7.8 Hz, 1H), 6.79 (d, J=7.8 Hz, 1H), 6.65-6.62 (m, 2H), 3.97 (t, J=5.9 Hz, 2H), 3.74 (t, J=6.8 Hz, 2H), 3.29 (s, 2H), 2.24 (br s, 4H), 2.03 (quintet, J=6.3 Hz, 2H), 1.46-1.41 (m, 4H), 1.35-1.33 (m, 2H).

### Step 3: 3-(3-(piperidin-1-ylmethyl)phenoxy)propan-1-amine (216)

Following the same procedure as described for compound 139 (scheme 16, step 5) but substituting phtalamide 138 for 215 to afford 216 (1.60 g, 91%) as a yellow oil. (DMSO-d6) δ(ppm): 7.17 (t, J=7.8 Hz, 1H), 6.81-6.74 (m, 3H), 3.97 (t, J=6.4 Hz, 2H), 3.34 (s, 2H), 2.66 (t, J=6.7 Hz, 2H), 2.27 (br s, 4H), 1.78-1.73 (m, 2H), 1.47-1.43 (m, 4H), 1.36-1.34 (m, 2H).

### Step 4: 2-(4-fluorobenzyloxy)-N-(3-(3-(piperidin-1-ylmethyl)phenoxy)propyl)acetamide (217a)

Following the same procedure as described for compound 1 (scheme 1, step 1) but substituting 140 for N-Boc-caproic acid and 216 for 3-phenyl aniline to afford 217a (170 mg, 33%) as a white oily solid.

(MeOD-d4) δ(ppm): 7.38 (dd, J=8.6, 5.3 Hz, 2H), 7.19 (t, J=7.8 Hz, 1H), 7.05 (t, J=8.8 Hz, 2H), 6.90-6.87 (m, 2H), 6.81-6.77 (m, 1H), 4.57 (s, 2H), 4.02 (t, J=6.1 Hz, 2H), 3.94 (s, 2H), 3.46-3.42 (m, 4H), 2.39 (br s, 4H), 1.99 (quintet, J=6.3 Hz, 2H), 1.57 (quintet, J=5.5 Hz, 4H), 1.45-1.44 (m, 2H).

### EXAMPLE 93b-g

Example 93b-g describe the preparation of compound 217b-g using the same procedures as described for compound 217a in Example 93a. Characterization data are presented in a Table 18.

TABLE 18

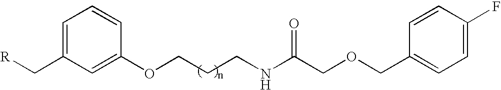

| Ex | Cpd | n | R or structure | Name | Characterization | Scheme |
|---|---|---|---|---|---|---|
| 93b | 217b | 0 | 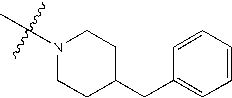 | N-(3-((4- benzylpiperidin-1- yl)methyl)phenoxy)propyl )-2-(4- fluorobenzyloxy)acetamide | (MeOD-d4) □(ppm): 7.39-7.36 (m, 2H), 7.25-7.02 (m, 6H), 6.91-6.87 (m, 2H), 6.82 (dd, J = 8.2, 1.8 Hz, 1 H), 4.56 (s, 2H), 4.02 (t, J = 8.1 Hz, 2H), 3.93 (s, 2H), 3.55 (s, 2H), 3.43 (t, J = 6.8 Hz, 2H), 2.93 (d, J = 11.9 Hz, 2H), 2.52 (d, J = 6.7 Hz, | |
| # 2H), 2.08 (t, J = 11.8 Hz, 2H), 1.99 (quintet, J = 6.3 Hz, 2H), 1.64-1.52 (m, 3H), 1.35-1.24 (m, 2H). LRMS (ESI): (calc) 504.3; (found) 505.5 (MH)⁺. | 32 | | | | | |
| 93c | 217c | 0 | 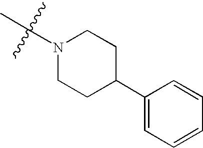 | 2-(4-fluorobenzyloxy)-N- (3-(3-((4- phenylpiperidin-1- yl)methyl)phenoxy)propyl) acetamide | (MeOD-d4)□(ppm) 7.39-7.36 (m, 2H), 7.27-7.19 (m, 5H), 7.17-7.13 (m, 1H), 7.05 (t, J = 11.2 Hz, 2H), 6.95-6.90 (m, 2H), 6.83-6.80 (m, 1H), 4.57 (s, 2H), 4.04 (t, J = 6.9 Hz, 2H), 3.94 (s, 2H), | |
| # 3.52 (s, 2H), 3.45 (t, J = 6.7 Hz, 2H), 3.00 (d, J = 12.1 Hz, 2H), 2.55-2.49 (m, 1H), 2.16-2.09 (m, 2H), 2.00 (quintet, J = 6.3 Hz, 2H), 1.80-1.71 (m, 4H). LRMS (ESI): (calc) 490.3; (found) 491.3 (MH)⁺. | 32 | | | | | |
| 93d | 217d | 0 | 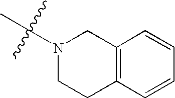 | N-(3-(3-((3,4- dihydroisoquinolin- 2(1H)- yl)methyl)phenoxy)propyl )-2-(4- fluorobenzyloxy)acetamide | (MeOD-d4) □(ppm): 7.36 (dd, J = 8.8, 5.5 Hz, 2H), 7.22 (t, J = 7.8 Hz, 1H), 7.10-6.94 (m, 8H), 6.82 (dd, J = 8.2, 1.8 Hz, 1H), 4.55 (s, 2H), 4.04 (t, J = 6.1 Hz, 2H), 3.93 (s, 2H), | |
| # 3.65 (s, 2H), 3.60 (s, 2H), 3.44 (t, J = 6.6 Hz, 2H), 2.88 (t, J = 6.0 Hz, 2H), 2.74 (t, J = 5.8 Hz, 2H), 1.99 (quintet, J = 6.3 Hz, 2H). LRMS (ESI): (calc) 462.2; (found) 463.2 (MH)⁺. | 32 | | | | | |
| 93e | 217e | 0 | 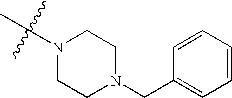 | N-(3-(3-((4- benzylpiperazin-1- yl)methyl)phenoxy)propyl )-2-(4- fluorobenzyloxy)acetamide | (MeOD-d4) □(ppm): 7.40-7.34 (m, 7H), 7.26 (t, J = 7.8 Hz, 1H), 7.06 (t, J = 8.8 Hz, 2H), 6.95-6.93 (m, 2H), 6.89-6.87 (m, 1H), 4.56 (s, 2H), 4.02 (t, J = 5.9 Hz, 2H), 3.93 (s, 2H), 3.88 (s, | |
| # 2H), 3.81 (s, 2H), 3.43 (t, J = 6.8 Hz, 2H), 2.88 (s, 8H), 2.01-1.96 (m, 2H). LRMS (ESI): (calc) 505.3; (found) 506.3 (MH)⁺. | 32 | | | | | |
| 93f | 217f | 1 | 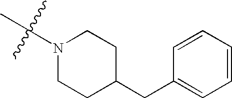 | N-(4-(3-((4- benzylpiperidin-1- yl)methyt)phenoxy)butyl) -2-(4- fluorobenzyloxy)acetamide | (MeOD-d4) □(ppm): 7.39 (dd, J = 8.6, 5.5 Hz, 2H), 7.24-7.03 (m, 8H), 6.88-6.78 (m, 3H), 4.54 (s, 2H), 3.97 (t, J = 5.8 Hz, 2H), 3.92 (s, 2H), 3.42 (s, 2H), 3.30 (t, J = 6.5 Hz, 2H), 2.86-2.82 | |
| # (m, 2H), 2.50 (d, J = 6.9 Hz, 2H), 1.92 (t, J = 9.8 Hz, 2H), 1.79-1.66 (m, 4H), 1.60-1.49 (m, 3H), 1.32-1.25 (m, 2H). LRMS (ESI): (calc) 518.3; (found) 519.3 (MH)⁺. | 32 | | | | | |

| | | | | |
|---|---|---|---|---|
| 93g | 217g | 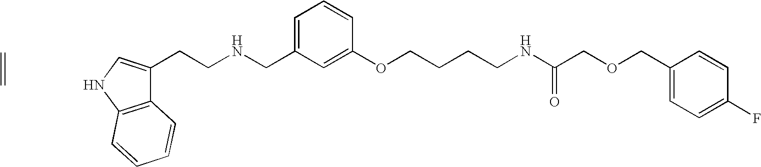 | N-(4-(3-((2-(1H-indol-3- yl)ethylamino)methyl) phenoxy)butyl)-2-(4- fluorobenzyloxy)acetamide | (MeOD-d4) □(ppm) ¹H: 7.52 (d, J = 7.8 Hz, 1H), 7.42-7.29 (m, 4H), 7.14-6.94 (m, 4H), 4.56 (s, 2H), 4.08 (s, 2H), 3.98 (t, J =6.1 Hz, 2H), 3.93 (s, 2H), 3.30-3.29 (m, 2H), 3.26-3.22 (m, 2H), |
| # 3.15-3.11 (m, 2H), 1.82-1.75 (m, 2H), 1.72-1.65 (m, 2H) LRMS (ESI): (calc) 503.3 (found) 504.6 (MH)⁺. | 32 | | | |

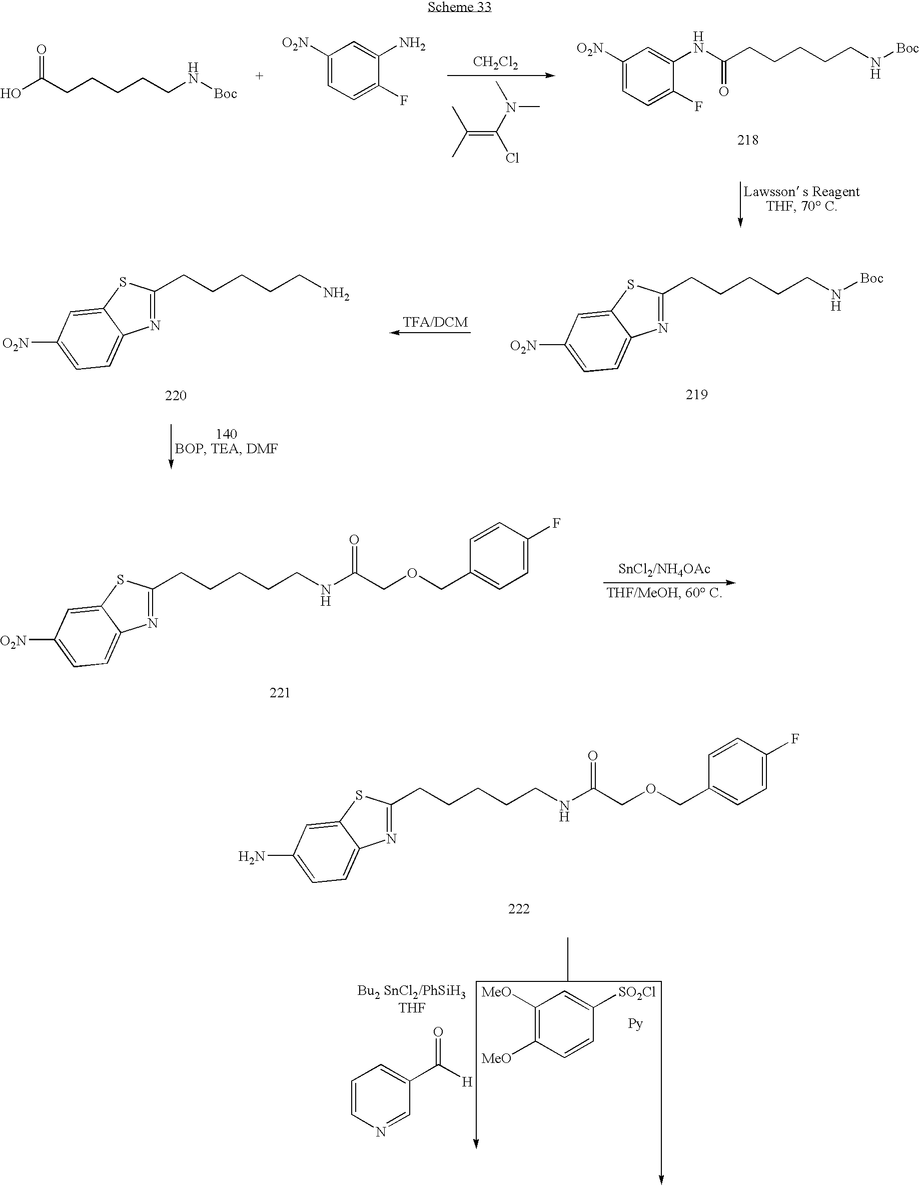

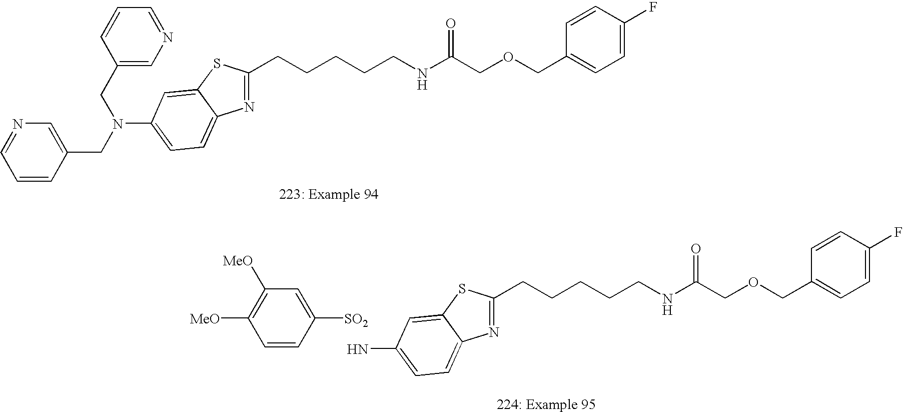

### EXAMPLE 94

### N-(5-(6-(bis(pyridin-3-ylmethyl)amino)benzo[d]thiazol-2-yl)pentyl)-2-(4-fluorobenzyloxy)acetamide (223)

### Step 1: tert-butyl 6-(2-fluoro-5-nitrophenylamino)-6-oxohexylcarbamate (218)

To a solution of 6-(tert-butoxycarbonylamino)hexanoic acid (4.95 g, 21.40 mmol) in CH₂Cl₂ at room temperature was added 1-chloro-N,N,2-trimethylprop-1-en-1-amine (3.40 ml, 25.68 mmol) and he reaction mixture was stirred for 1 hour. Then 2-fluoro-5-nitrobenzenamine (3.34 g, 21.40 mmol) was added. The reaction was stirred for 20 minutes and quenched with water. Aqueous extraction was performed with ethyl acetate. The organic layer was dried (MgSO₄) and concentrated to afford 218 (3.08 g, 39%) as a white solid.

(MeOD-d4) □(ppm): 9.05 (dd,J=2.7, 6.7 Hz, 1H), 8.06-8.02 (m, 1H), 7.39 (t, J=9.2 Hz, 1H), 3.04 (t, J=6.8 Hz, 2H), 2.49 (t, J=7.4 Hz, 2H), 1.74-1.69 (m, 2H), 1.54-1.48 (m, 2H), 1.41 (s, 11H).

### Step 2: tert-butyl 5-(6-nitrobenzo[d]thiazol-2-yl)pentylcarbamate (219)

Following the same procedure as described for compound 10 (scheme 1, step 5) but substituting 218 for 7b to afford 219 (1.97 g, 65%) as a yellow solid.

(DMSO-d6) □(ppm):8.70 (d, J=2.2 Hz, 1H), 8.35-8.33 (m, 1H), 8.25-8.22 (m, 1H), 6.78 (t, J=5.5 Hz, 1H), 3.15 (t, J=7.4 Hz, 2H), 2.88 (dd, J=6.3, 12.5 Hz, 2H), 1.82-1.78 (m, 2H), 1.42-1.37 (m, 2H), 1.32 (s, 9H).

### Step 3: 5-(6-nitrobenzo[d]thiazol-2-yl)pentan-1-amine (220)

Following the same procedure as described for compound 2 (step 2, scheme 1, example 1) but substituting 219 for 1 to afford 220 (1.32 g, 92%) as a red oil.

(MeOD-d4) □(ppm): 8.74 (d, J=2.3 Hz, 1H), 8.30-8.26 (m, 1H), 8.20-8.17 (m; 1H), 3.22 (t, J=7.6 Hz, 2H), 2.70 (t, J=7.0 Hz, 2H), 1.98-1.91 (m, 2H), 1.61-1.48 (m, 4H).

### Step 4: 2-(4-fluorobenzyloxy)-N-(5-(6-nitrobenzo[d]thiazol-2-yl)pentyl)acetamide (221)

Following the same procedure as described for compound 1 (scheme 1, step 1) but substituting 140 for N-Boc-caproic acid and 220 for 3-phenyl aniline to afford 221 (1.01 g, 47%) as an orange oil.

(MeOD-d4) □(ppm): 8.71 (d, J=2.2 Hz, 1H), 8.27-8.24 (m, 1H), 8.17-8.14 (m, 1H), 7.39-7.34 (m, 2H), 7.06 (t, J=8.8 Hz, 2H), 4.52 (s, 2H), 3.91 (s, 2H), 3.25 (t, J=6.8 Hz, 2H), 3.20 (t, J=7.4 Hz), 1.94 (m, 2H), 1.62-1.56 (m, 2H), 1.50-1.44 (m, 2H).

### Step 5: N-(5-(6-aminobenzo[d]thiazol-2-yl)pentyl)-2-(4-fluorobenzyloxy)acetamide (222)

Following the same procedure as described for compound 163a (scheme 20, example 79a) but substituting 221 for 162a and employing 3:1 methanol:THF as solvent to afford 222 (753 mg, 80%) as an orange oil.

(DMSO-d6) □(ppm): 8.69 (d, J=2.2 Hz, 1H), 8.34-8.32 (m, 1H), 8.24-8.21 (m, 1H), 7.80 (t, J=5.7 Hz, 1H), 7.40-7.36 (m, 2H), 7.17-7.13 (m, 2H), 4.46 (s, 2H), 3.84 (s, 2H), 3.16 (t, J=7.4 Hz, 2H), 3.08 (dd, J=6.7, 13.1 Hz, 2H), 1.84-1.80 (m, 2H), 1.49-1.45 (m, 2H), 1.37-1.32 (m, 2H).

### Step 6: N-(5-(6-(bis(pyridin-3-ylmethyl)amino)benzo[d]thiazol-2-yl)pentyl)-2-(4-fluorobenzyloxy)acetamide (223)

Following the same procedure as described for compound 75 (step 6, scheme 5, example 55) but substituting 222 for compound 74 and 2 equivalents of nicotinaldehyde for 71 to afford 223 (92 mg, 25%) as a colourless oil.

(MeOD-d4) □(ppm): 8.49-8.48 (m, 2H), 8.41 (d, J=4.9 Hz, 2H), 7.88 (s, 1H), 7.77 (d, J=8.0 Hz, 2H), 7.66 (d, J=8.8 Hz, 1H), 7.40-7.34 (m, 4H), 7.18 (d, J=2.5 Hz, 1H), 7.08-7.00 (m, 3H), 4.84 (s, 4H), 4.50 (s, 2H), 3.89 (s, 2H), 3.21 (dd, J=6.8, 13.3 Hz, 2H), 3.03 (t, J=7.4 Hz, 2H), 1.85-1.81 (m, 2H), 1.57-1.52 (m, 2H), 1.43-1.37 (m, 2H).

### EXAMPLE 95

### N-(5-(6-(3,4-dimethoxyphenylsulfonamido)benzo[d]thiazol-2-yl)pentyl)-2-(4-fluorobenzyloxy)acetamide (224)

### Step 6: N-(5-(6-(3,4-dimethoxyphenylsulfonamido)benzo[d]thiazol-2-yl)pentyl)-2-(4-fluorobenzyloxy)acetamide (224)

To a solution of 222 (251 mg, 0.63 mmol) and DMAP (8 mg, 0.063 mmol) in pyridine was added 3,4-dimethoxybenzene-1-sulfonyl chloride (149 mg, 0.63 mmol). The reaction was stirred at room temperature for 2 hours and quenched with water. Aqueous extraction was performed with dichloromethane. The organic layer was dried (MgSO₄) and concentrated. The residue was chromatographed on prep HPLC (with gradient of ethyl acetate 50-100% in hexanes in silicagel column) to afford 224 (110 mg, 30%) as a white solid.

(MeOD-d4) □(ppm): 7.75 (d, J=8.6 Hz, 1H), 7.63 (d, J=1.8 Hz, 1H), 7.37-7.33 (m, 3H), 7.24 (d, J=2.2 Hz, 1H), 7.16 (dd, J=8.6, 2.2 Hz, 1H), 7.08-7.03 (t, J=9.0 Hz, 2H), 6.94 (d, J=8.6 Hz, 1H), 4.50 (s, 2H), 3.89 (s, 2H), 3.80 (s, 3H), 3.73 (s, 3H), 3.23 (t, J=6.8 Hz, 2H), 3.08 (t, J=7.4 Hz, 2H), 1.90-1.82 (m, 2H), 1.58-1.53 (m, 2H), 1.45-1.39 (m, 2H).

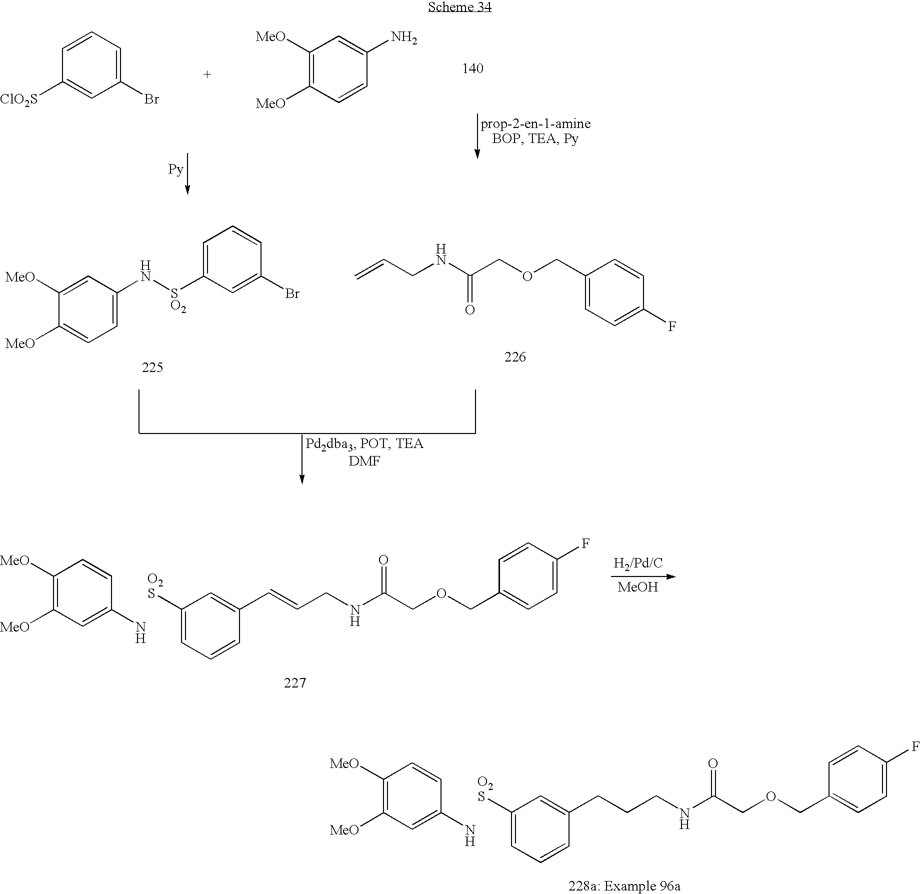

### EXAMPLE 96a

### N-(3-(3-(N-(3,4-dimethoxyphenyl)sulfamoyl)phenyl)propyl)-2-(4-fluorobenzyloxy)acetamide (228a)

### Step 1: 3-bromo-N-(3,4-dimethoxyphenyl)benzenesulfonamide (225)

Following the same procedure as described for compound 54 (scheme 2, example 47) but substituting 3,4-dimethoxyaniline for amine 2 and 3-bromobenzene-1-sulfonyl chloride for 2-(benzyloxy)acetyl chloride along with catalytic DMAP to afford 225 (900 mg, 80%) as a white solid. (DMSO-d6) □(ppm): 10.00 (s,1H), 7.83-7.79 (m, 2H), 7.66-7.63 (m, 1H), 7.51-7.46 (m, 1H), 6.80 (d, J=8.8 Hz, 1H), 6.65 (d, J=2.3 Hz, 1H), 6.53 (dd, J=8.4, 2.4 Hz, 1H), 3.65 (s, 3H), 3.62 (s, 3H).

### Step 2: N-allyl-2-(4-fluorobenzyloxy)acetamide (226)

Following the same procedure as described for compound 1 (scheme 1, step 1) but substituting 140 for N-Boc-caproic acid and allyl amine for 3-phenyl aniline to afford 226 (750 mg, 62%) as a colourless oil. (DMSO-d6) □(ppm): 7.97 (s, 1H), 7.42 (dd, J=8.6, 5.8 Hz, 2H), 7.17 (t, J=8.8 Hz, 2H), 5.83-5.73 (m, 1H),-5.11-5.00 (m, 2H), 4.50 (s, 2H), 3.90 (s, 2H), 3.73-3.69 (m, 2H).

### Step 3: (E)-N-(3-(3-(N-(3,4-dimethoxyphenyl)sulfamoyl)phenyl)allyl)-2-(4-fluorobenzyloxy)acetamide (227)

Following the same procedure as described for compound 184 (scheme 25, step 2, example 85a) but substituting 225 for compound 181 and 226 for vinyl phthalamide to afford 227 (250 mg, 28%) as a colourless oily solid. (MeOD-d4) □(ppm): 7.67 (t, J=1.8 Hz, 1H), 7.58-7.53 (m, 2H), 7.44-7.39 (m, 3H), 7.08 (t, J=9.0 Hz, 2H), 6.76 (d, J=8.6 Hz, 1H), 6.65 (d, J=2.4 Hz, 1H), 6.57-6.49 (m, 2H), 6.27-6.20 (m, 1H), 4.60 (s, 2H), 4.00-3.98 (m, 4H), 3.73 (s, 3H), 3.68 (s, 3H).

### Step 4: N-(3-(3-(N-(3,4-dimethoxyphenyl)sulfamoyl)phenyl)propyl)-2-(4-fluorobenzyloxy)acetamide (228a)

Following the same procedure as described for compound 74 (scheme 5, example 55) but substituting 227 for 73 to afford 228 (160 mg, 80%) as a white solid.

(MeOD-d4) □(ppm): 7.53 (dt, J=6.9, 2.0 Hz, 1H), 7.48 (s, 1H), 7.42-7.34 (m, 4H), 7.08 (t, J=11.2 Hz, 2H), 6.74 (d, J=8.6 Hz, 1H), 6.63 (d, J=2.3 Hz, 1H), 6.56 (dd, J=8.4, 2.4 Hz, 1H), 4.56 (s, 2H), 3.93 (s, 2H), 3.71 (s, 3H), 3.66 (s, 3H), 3.15 (t, J=7.0 Hz, 2H), 2.61 (t, J=7.6 Hz, 2H), 1.72 (quintet, J=7.2 Hz, 2H).

### EXAMPLE 96b,c

Example 96b,c describe the preparation of compound 228b,c using the same procedures as described for compound 228a in Example 96a. Characterization data are presented in a Table 19.

TABLE 19

| Ex | Cpd | structure | Name | Characterization | Scheme |
|---|---|---|---|---|---|
| 96b | 228b | 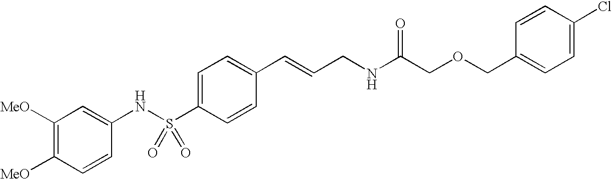 | (E)-2-(4- chlorobenzyloxy)-N-(3- (4-(N-(3,4- dimethoxyphenyl)sulfamoly) phenyl)allyl)acetamide | (MeOD-c14) d(ppm): d(ppm): 7.62 (d, J = 8.6 Hz, 2H), 7.47 (d, J = 8.6 Hz, 2H), 7.40-7.34 (m, 4H), 6.76 (d, J = 8.6 Hz, 1H), 6.67 (d, J = 2.5 Hz, 1H), 6.55 (dd, J = 8.6 and 2.3 Hz, 1H), 6.53 (bd, | |
| # J = 16 Hz, 1H), 6.36 (dt, J = 16, 5.7Hz, 1H), 4.60 (s, 2H), 4.02 (m, 2H), 4.00 (s, 2H), 3.74 (s, 3H), 3.69 (s, 3H). LRMS (ESI): 530.1 (calc) 529.0 (M-) (found). | 34 (step 1-4) | | | | |
| 96c | 228c | 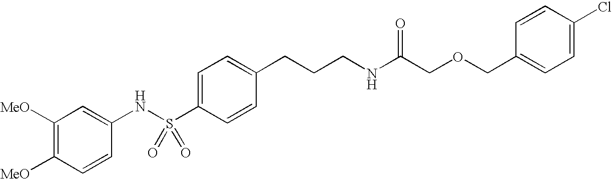 | 2-(4-chlorobenzyloxy)- N-(3-(4-(N-(3,4- dimethoxyphenyl)sulfamoyl) phenyl)propyl)acetamide | (DMSO-d6) d(ppm): 9.84 (s, 1H), 7.87 (t, J = 6.5 Hz, 1H), 7.59 (d, J = 8.4 Hz, 2H), 7.42-7.33 (m, 6H), 6.76 (d, J = 8.6 Hz, 1H), 6.62 (d, J = 2.5 Hz, 1H), 6.52 (dd, J = 8.6, 2.5 Hz, 1H), 4.50 (s, 2H), | |
| # 3.86 (s, 2H), 3.63 (s, 3H), 3.59 (s, 3H), 3.07 (q, J = 6.1 Hz, 2H), 2.58 (t, J = 7.8 Hz, 2H), 1.67 (qi, J = 7.2 Hz, 2H). LRMS (ESI): 532.1 (calc) 531.0 (M-) (found). | 34 (step 4) | | | | |

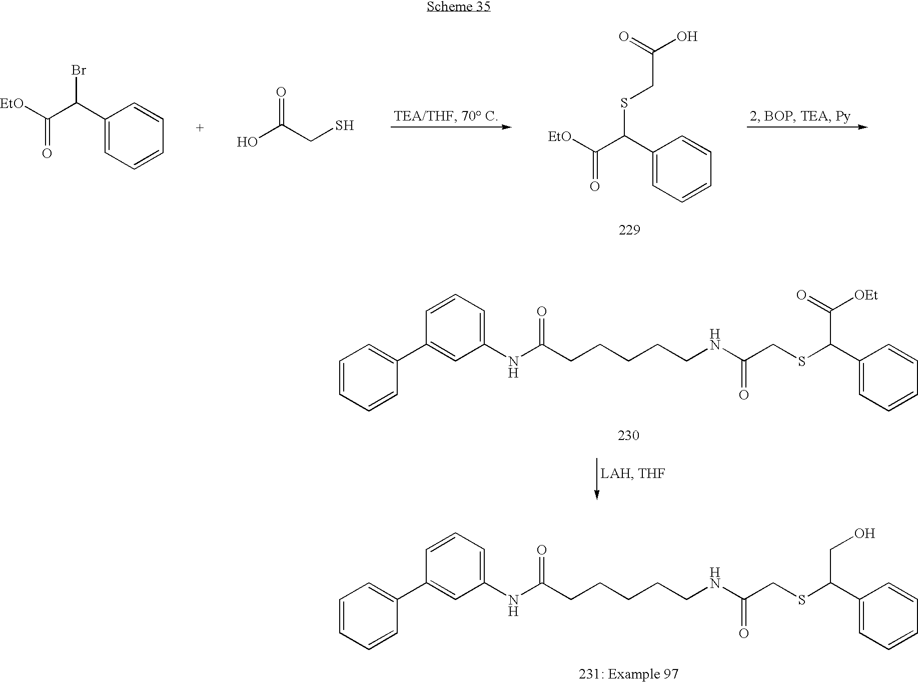

### EXAMPLE 97

### N-(biphenyl-3-yl)-6-(2-(2-hydroxy-1-phenylethylthio)acetamido)hexanamide (231)

### Step 1: 2-(2-ethoxy-2-oxo-1-phenylethylthio)acetic acid (229)

Following the same procedure as described for compound 4 (step 4, scheme 1, example 1) but substituting 2-mercaptoacetic acid for methyl 2-mercaptoacetate and ethyl 2-bromo-2-phenylacetate for 3b and heating to 80° C. to afford 229 (1.05 g, 99%) as a colourless oil. (DMSO-d6) δ (ppm): 12.71 (br s, 1H), 7.39-7.29 (m, 5H), 4.87 (s, 1H), 4.15-4.04 (m, 2H), 3.28 (d, J=15.3 Hz, 1H), 3.18 (d, J=15.4 Hz, 1H), 1.17-1.11 (m, 3H).

### Step 2: ethyl 2-(2-(6-(biphenyl-3-ylamino)-6-oxohexylamino)-2-oxoethylthio)-2-phenylacetate (230)

Following the same procedure as described for compound 1 (scheme 1, step 1) but substituting 229 for N-Boc-caproic acid and 2 for 3-phenyl aniline to afford 230 (520 mg, 78%) as a colourless oil.

(MeOD-d4) δ(ppm): 7.85 (s, 1H), 7.61-7.58 (m, 2H), 7.53 (dt, J=7.3, 1.7 Hz, 1H), 7.44-7.26 (m, 10H), 4.82 (s, 1H), 4.18-4.10 (m, 2H), 3.22-3.04 (m, 4H), 2.41 (t, J=7.4 Hz, 2H), 1.73 (quintet, J=7.2 Hz, 2H), 1.56-1.50 (m, 2H), 1.43-1.41 (m, 2H), 1.19 (t, J=7.2 Hz, 3H).

### Step 3: N-(biphenyl-3-yl)-6-(2-(2-hydroxy-1-phenylethylthio)acetamido)hexanamide (231)

Following the same procedure as described for compound 12 (scheme 1, step 5, example) but substituting 230 for 7c and employing 2 equivalents of LiAlH₄ with 1 h stir time to afford 231 (25 mg, 16%) as a colourless oil.

(MeOD-d4) δ(ppm): 7.85 (t, J=1.6 Hz, 1H), 7.60-7.57 (m, 2H), 7.53 (dt, J=7.5, 2.0 Hz, 1H), 7.44-7.21 (m, 10H), 4.02 (t, J=7.0 Hz, 1H), 3.85-3.83 (m, 1H), 3.16-3.12 (m, 3H), 2.98 (d, J=15.1 Hz, 1H), 2.40 (t, J=7.4 Hz, 2H), 1.73 (quintet, J=7.6 Hz, 2H), 1.55-1.49 (m, 2H), 1.44-1.40 (m, 2H).

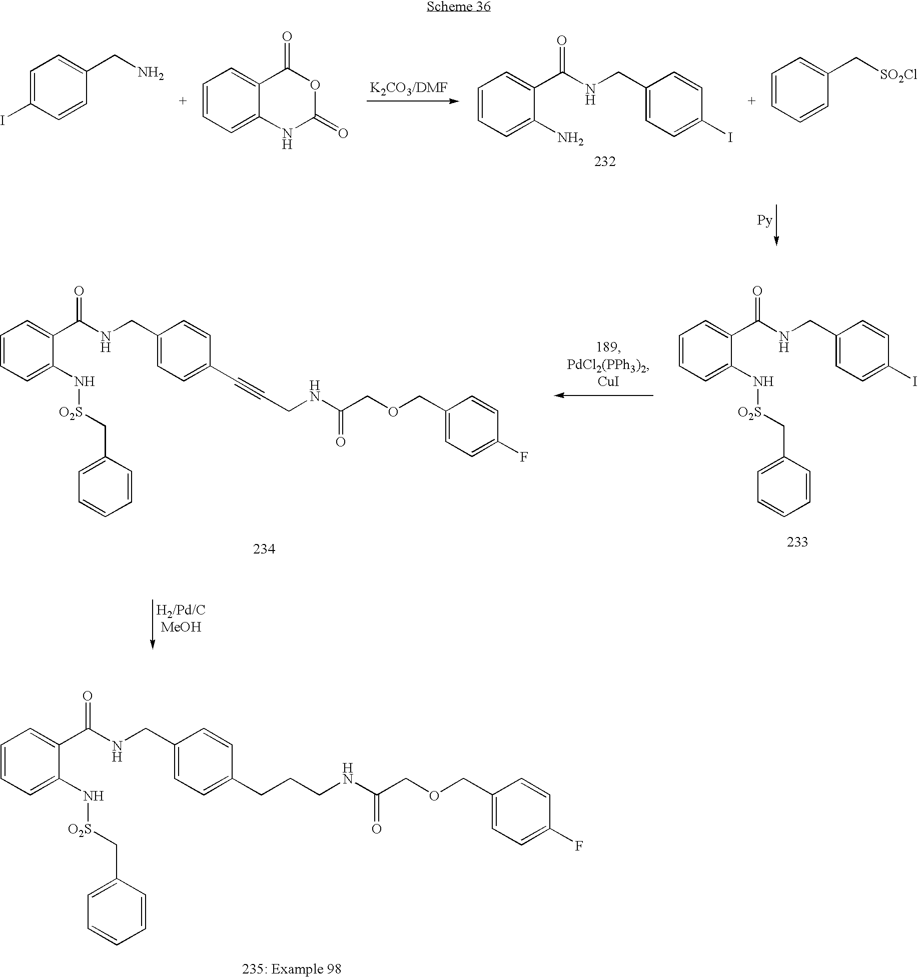

### EXAMPLE 98

### N-(4-(3-(2-(4-fluorobenzyloxy)acetamido)propyl)benzyl)-2-(phenylmethylsulfonamido)benzamide (235)

### Step 1: 2-amino-N-(4-iodobenzyl)benzamide (232)

4-iodobenzylamine hydrochloride (1.07 g, 3.98 mmol) was dissolved in DMF (15 mL) with 1H-benzo[d][1,3]oxazine-2,4-dione (650 mg, 3.98 mmol). K₂CO₃ (1.38 g, 9.95 mmol) was added and the reaction stirred at 50° C. for 1 h. Aqueous extraction was performed with ethyl acetate and water and the organic layer was separated, dried with MgSO₄ and the concentrated. The residue was triturated with a 50% hexanes:ethyl acetate solvent mixture to afford 233 (820 mg, 50%) as a beige solid.

(DMSO-d6) δ(ppm): 8.78 (t, J=5.9 Hz, 1H), 7.77-7.63 (m, 2H), 7.53-7.50 (m, 1H), 7.25-7.22 (m, 3H), 7.14-7.09 (m, 1H), 6.68-6.47 (m, 1H), 6.42 (br s, 2H), 4.34 (d, J=6.0 Hz, 2H).

### Step 2: N-(4-iodobenzyl)-2-(phenylmethylsulfonamido)benzamide (233)

Following the same procedure as described for compound 54 (scheme 2, example 47) but substituting 232 for amine 2 and phenylmethanesulfonyl chloride for 2-(benzyloxy)acetyl chloride to afford 233 (640 mg, 56%) as a white solid.

(DMSO-d6) δ(ppm): 11.16 (s, 1H), 9.42 (t, J=5.3 Hz, 1H), 7.85 (d, J=7.8 Hz, 1H), 7.68 (d, J=8.2 Hz, 2H), 7.53-7.47 (m, 2H), 7.31-7.09 (m, 8H), 4.58 (s, 2H), 4.33 (d, J=5.9 Hz, 2H).

### Step 3: N-(4-(3-(2-(4-fluorobenzyloxy)acetamido)prop-1-ynyl)benzyl)-2-(phenylmethylsulfonamido)benzamide (234)

Following the same procedure as described for compound 182a (scheme 25, example 84a) but substituting 233 for 181 and 189 for methyl 3-(2-oxo-2-(prop-2-ynylamino)ethylthio)propanoate to afford 234 which was used in the next step without further purification.

(MeOD-d4) δ(ppm): 7.73 (dd, J=7.8, 1.4 Hz, 1H), 7.66 (dd, J=8.4, 1.2 Hz, 1H), 7.49-7.44 (m, 1H), 7.43-7.36 (m, 4H), 7.30-7.12 (m, 8H), 7.07 (t, J=8.8 Hz, 2H), 4.58 (s, 2H), 4.43 (s, 2H), 4.40 (s, 2H), 4.23 (s, 2H), 3.98 (s, 2H).

### Step 4: N-(4-(3-(2-(4-fluorobenzyloxy)acetamido)propyl)benzyl)-2-(phenylmethylsulfonamido)benzamide (235)

Following the same procedure as described for compound 74 (scheme 5, example 55) but substituting 234 for 73 to afford 235 (90 mg, 13% (2 steps)) as a white foam.

(MeOD-d4) δ(ppm): 7.71 (dd, J=8.0, 1.6 Hz, 1H), 7.64 (dd, J=8.4, 1.0 Hz, 1H), 7.46-7.42 (m, 1H), 7.38 (dd, J=8.6, 5.5 Hz, 2H), 7.28-7.10 (m, 10H), 7.06 (t, J=8.8 Hz, 2H), 4.53 (s, 2H), 4.39 (s, 2H), 4.38 (s, 2H), 3.89 (s, 2H), 3.22 (t, J=7.0 Hz, 2H), 2.59 (t, J=7.4 Hz, 2H), 1.78 (quintet, J=7.2 Hz, 2H).

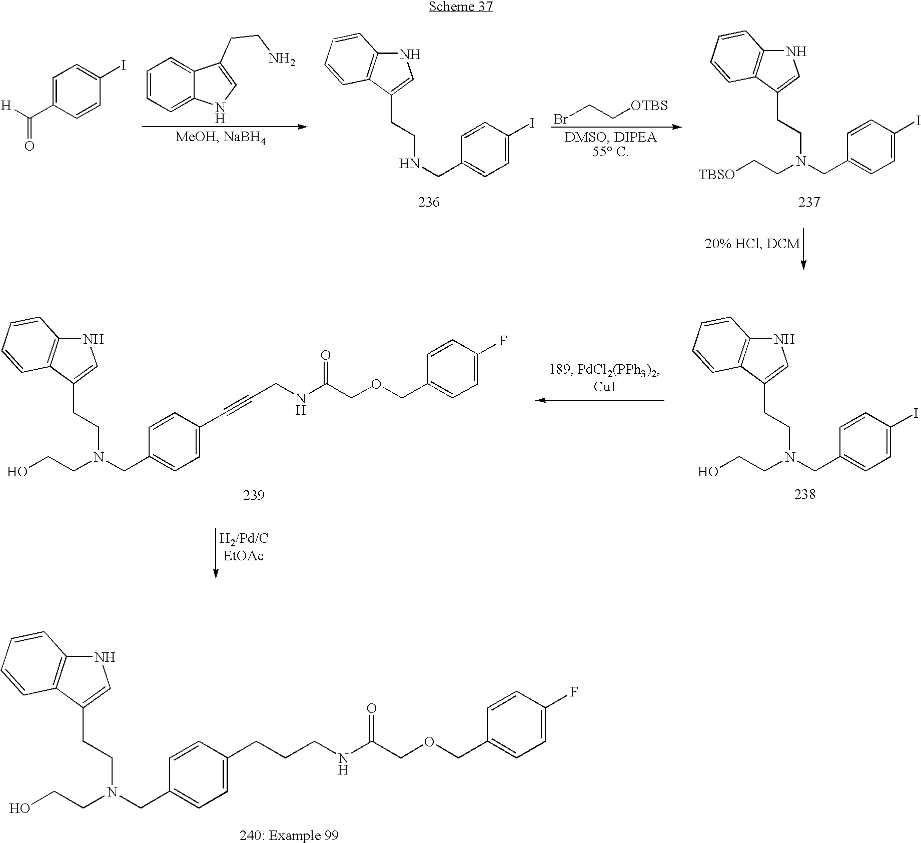

### EXAMPLE 99

### N-(3-(4-(((2-(1H-indol-3-yl)ethyl)(2-hydroxyethyl)amino)methyl)phenyl)propyl)-2-(4-fluorobenzyloxy)acetamide (240)

### Step 1: 2-(1H-indol-3-yl)-N-(4-iodobenzyl)ethanamine (236)

Following the same procedure as described for compound 196a (step 5, scheme 27, example 87a) but substituting 4-iodobenzaldehyde for 195a to afford 236 (950 mg, 62%) as an off-white solid.

(DMSO-d6) δ(ppm): 10.75 (s, 1H), 7.64-7.60 (m, 2H), 7.45 (d, J=7.8 Hz, 1H), 7.29 (d, J=8.0 Hz, 1H), 7.13-7.08 (m, 3H), 7.02 (td, J=7.0, 1.0 Hz, 1H), 6.95-6.90 (m, 1H), 3.66 (s, 2H), 2.82-2.70 (m, 4H).

### Step 2: N-(2-(1H-indol-3-yl)ethyl)-2-(tert-butyldimethylsilyloxy)-N-(4-iodobenzyl)ethanamine (237)

To a solution of 236 (925 mg, 2.46 mmol) in DMSO (10 mL) was added (2-Bromoethoxy)(tert-butyl)dimethylsilane (577 μL, 2.71 mmol) and DIPEA (556 μL, 3.20 mmol). The reaction heated to 55° C. and allowed to stir for 16 h. Aqueous extraction was performed with ethyl acetate and water and the organic layer was separated, dried with MgSO₄ and concentrated. The residue was purified was in prep HPLC (gradient ethyl acetate (0-50%) in: hexanes) to afford 237 (520 mg, 40%) as a clear oil.

(DMSO-d6) δ(ppm): 10.75 (s, 1H), 7.65-7.62 (m, 2H), 7.35-7.29 (m, 2H), 7.15 (d, J=8.2 Hz, 2H), 7.09 (d, J=2.4 Hz, 1H), 7.05-7.01 (m, 1H), 6.93-6.89 (m, 1H), 3.67 (s, 2H), 3.64 (t, J=6.5 Hz, 2H), 2.84-2.71 (m, 4H), 2.63 (t, J=6.5 Hz, 2H), 0.83 (s, 9H), 0.00 (s, 6H).

### Step 3: N-(3-(4-(((2-(1H-indol-3-yl)ethyl)(2-(tert-butyidimethylsilyloxy)ethyl)amino)methyl)phenyl)prop-2-ynyl)-2-(4-fluorobenzyloxy)acetamide (238)

Following the same procedure as described for compound 182a (scheme 25, example 84a) but substituting 237 for 181 and 189 for methyl 3-(2-oxo-2-(prop-2-ynylamino)ethylthio)propanoate to afford 238 (150 mg, 25%) as a yellow oil.

(DMSOD6) δ(ppm): 10.75 (s, 1H), 8.41-8.39 (m, 1H), 7.45 (dd, J=8.6, 5.5 Hz, 2H), 7.37-7.29 (m, 6H), 7.19 (t, J=9.0 Hz, 2H), 7.09 (d, J=2.3 Hz, 1H), 7.05-7.01 (m, 1H), 6.94-6.89 (m, 1H), 4.54 (s, 2H), 4.15 (d, J=5.8 Hz, 2H), 3.96 (s, 2H), 3.73 (s, 2H), 3.65 (t, J=6.4 Hz, 2H), 2.86-2.74 (m, 4H), 2.64 (t, J=6.3 Hz, 2H), 0.84 (s, 9H), 0.00 (s, 6H).

### Step 4: N-(3-(4-(((2-(1H-indol-3-yl)ethyl)(2-hydroxyethyl)amino)methyl)phenyl)prop-2-ynyl)-2-(4-fluorobenzyloxy)acetamide (239)

238 (130 mg, 0.207 mmol) was dissolved in 20% HCL in ethanol (20 mL). The reaction was stirred at room temperature for 1 h and quenched with saturated NaHCO₃ solution Aqueous extraction was performed with ethyl acetate and water and the organic layer was separated, dried with MgSO₄ and concentrated. The residue was purified in silica gel column chromatography with ethyl acetate (5%) in methanol to afford 239 (55 mg, 52%) as a yellow oil.

(MeOD-d4) δ(ppm): 7.40 (dd, J=8.8, 5.5 Hz, 2H), 7.37-7.27 (m, 6H), 7.09-7.01 (m, 3H), 6.97 (s, 1H), 6.95-6.90 (m, 1H), 4.57 (s, 2H), 4.23 (s, 2H), 3.98 (s, 2H), 3.72 (s, 2H), 3.62 (t, J=6.3 Hz, 2H), 2.94-2.89 (m, 2H), 2.82-2.77 (m, 2H), 2.72 (t, J=6.2 Hz, 2H).

### Step 5: N-(3-(4-(((2-(1H-indol-3-yl)ethyl)(2-hydroxyethyl)amino)methyl)phenyl)propyl)-2-(4-fluorobenzyloxy)acetamide (240)

Following the same procedure as described for compound 74 (scheme 5, example 55) but substituting 234 for 73 and ethyl acetate for methanol to afford 240 (4 mg, 20%) as a clear oil.

(MeOD-d4) δ(ppm): 7.42-7.37 (m, 3H), 7.34-7.30 (m, 3H), 7.22 (d, J=7.8 Hz, 2H), 7.11-7.04 (m, 4H), 6.97-6.93 (m, 1H), 4.56 (s, 2H), 4.20 (s, 2H), 3.93 (s, 2H), 3.82 (t, J=5.5 Hz, 2H), 3.27-3.11 (m, 8H), 2.64 (t, J=7.4 Hz, 2H), 1.83 (quintet, J=7.2 Hz, 2H).

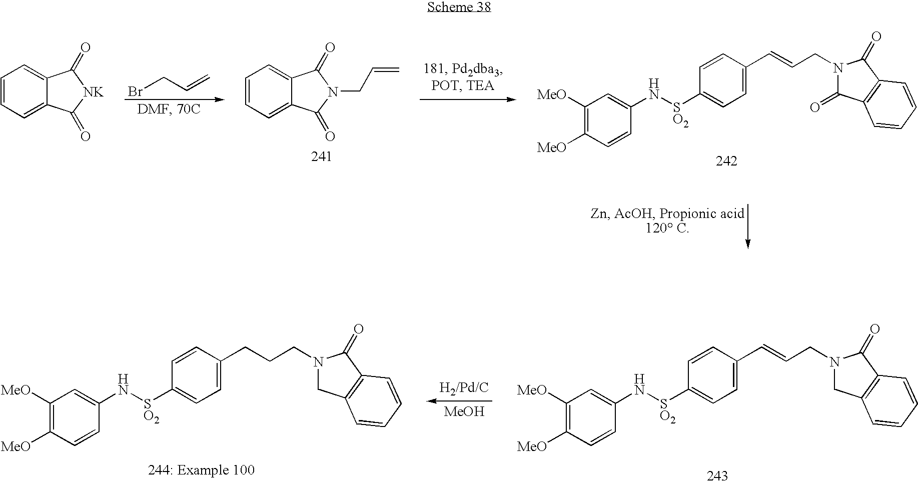

### EXAMPLE 100

### N-(3,4-dimethoxyphenyl)-4-(3-(1-oxoisoindolin-2-yl)propyl)benzenesulfonamide (244)

### Step 1: 2-allylisoindoline-1,3-dione (241)

To a solution of phtalamide potassium salt (6 g, 32.4 mmol) in DMF (40 mL), was added allyl bromide (2.78 mL, 32.4 mmol). The solution was heated to 70° C. and stirred for 16 h. The DMF was removed by rotary evaporation and aqueous extraction was performed with ethyl acetate and water and the organic layer was separated, dried with NaSO₄ and concentrated. The residue was purified by silica gel column chromatography with a gradient of ethyl acetate (20-80%) in hexanes to afford 241 (3.4 g, 57%) as a white solid.

(DMSO-d6) δ(ppm): 7.89-7.80 (m, 4H), 5.90-5.80 (m, 1H), 5.12 (t, J=1.6 Hz, 1H), 5.10-5.06 (m, 1H), 4.16 (dt, J=5.1, 1.8 Hz, 2H).

### Step 2: (E)-N-(3,4-dimethoxyphenyl)-4-(3-(1,3-dioxoisoindolin-2-yl)prop-1-enyl)benzenesulfonamide (242)

Following the same procedure as described for compound 184 (scheme 25, step 2, example 85a) but substituting 241 for vinyl phthalamide to afford 242 (1.0 g, 18%) as a white solid.

(DMSO-d6) δ(ppm): 9.84 (s, 1H), 7.90-7.81 (m, 4H), 7.68-7.53 (m, 4H), 6.74 (d, J=8.8 Hz, 1H), 6.64 (d, J=2.5 Hz, 1H), 6.59-6.42 (m, 3H), 4.35-4.33 (m, 2H), 3.62 (s, 3H), 3.59 (s, 3H).

### Step 3: (E)-N-(3,4-dimethoxyphenyl)-4-(3-(1-oxoisoindolin-2-yl)prop-1-enyl)benzenesulfonamide (243)

To a solution of 242 (235 mg, 0.492 mmol) in acetic acid (25 mL), was added zinc dust (1.8 g, 27.6 mmol) and propionic acid (2.5 mL). The suspension was heated to 120° C. and stirred for 3 h. The zinc dust was filtered off through Celite and the acetic acid removed via rotary evaporation. Aqueous extraction was performed with ethyl acetate and water. The organic layer was separated, dried with NaSO₄ and concentrated. The residue was purified by silica gel column chromatography with a gradient of ethyl acetate (33-100%) in hexanes to afford 243 (140 mg, 62%) as a colourless oil.

(MeOD-d4) δ(ppm): 7.78 (d, J=7.6 Hz, 1H), 7.63-7.48 (m, 7H), 6.75 (d, J=8.6 Hz, 1H), 6.68 (d, J=2.6 Hz, 1H), 6.64-6.61 (m, 1H), 6.54 (dd, J=8.6, 2.5 Hz, 1H), 6.50-6.42 (m, 1H), 4.52 (s, 2H), 4.42-4.40 (m, 2H), 3.73 (s, 3H), 3.69 (s, 3H).

### Step 4: N-(3,4-dimethoxyphenyl)-4-(3-(1-oxoisoindolin-2-yl)propyl)benzenesulfonamide (244)

Following the same procedure as described for compound 74 (scheme 5, example 55) but substituting 243 for 73 to afford 244 (65 mg, 59%) as a light yellow oily solid.

(MeOD-d4) δ(ppm): 7.75-7.72 (m, 1H), 7.60-7.45 (m, 5H), 7.32 (d, J=8.4 Hz, 2H), 6.74 (d, J=8.6 Hz, 1H), 6.65 (d, J=2.3 Hz, 1H), 6.55 (dd, J=8.6, 2.5 Hz, 1H), 4.46 (s, 2H), 3.71 (s, 3H), 3.67 (s, 3H), 3.62 (t, J=7.0 Hz, 2H), 2.69 (t, J=7.4 Hz, 2H), 1.98 (quintet, J=5.5 Hz, 2H).

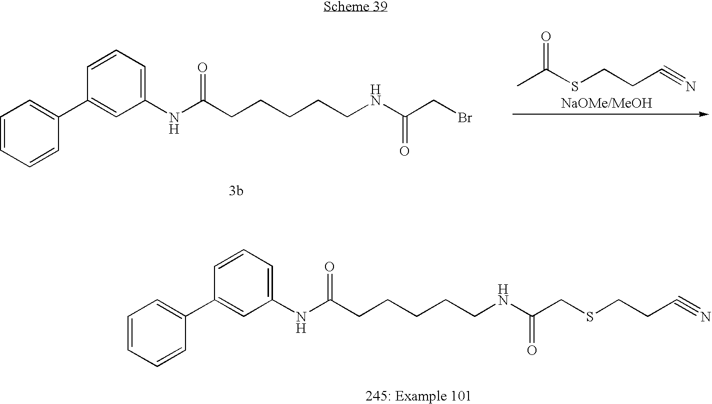

### EXAMPLE 101

### N-(biphenyl-3-yl)-6-(2-(2-cyanoethylthio)acetamido)hexanamide (245)

### Step 1: N-(biphenyl-3-yl)-6-(2-(2-cyanoethylthio)acetamido)hexanamide (245)

To a solution of 3b (0.250 mg, 0.622 mmol) in methanol (5.0 mL) with 3-(acetylthio)propionitrile) (0.088 mg, 0.684 mmol) was added 25% sodium methoxide in methanol (40 μl, 0.684 mmol). After stirring 30 minutes at room temperature, the solution was diluted in ethyl acetate and washed with water and brine. The organic phase was dried (Na₂SO₄), filtered and concentrated. The residue was purified by silica gel column chromatography with ethyl acetate to afford 245 (143 mg, 56%) as an off-white solid. (MeOD-d4) δ(ppm) ¹H: 7.84 (m, 1H), 7.61-7.58 (m, 2H), 7.53-7.51 (m, 1H), 7.44-7.31 (m, 5H), 3.25-3.21 (m, 4H), 2.86-2.83 (m, 2H), 2.78-2.75 (m, 2H), 2.43 (t, J=7.6 Hz, 2H), 1.80-1.72 (m, 2H), 1.63-1.56 (m, 2H), 1.49-1.43 (m, 2H). LRMS (ESI): (calc) 409.2 (found) 410.3 (MH)⁺.

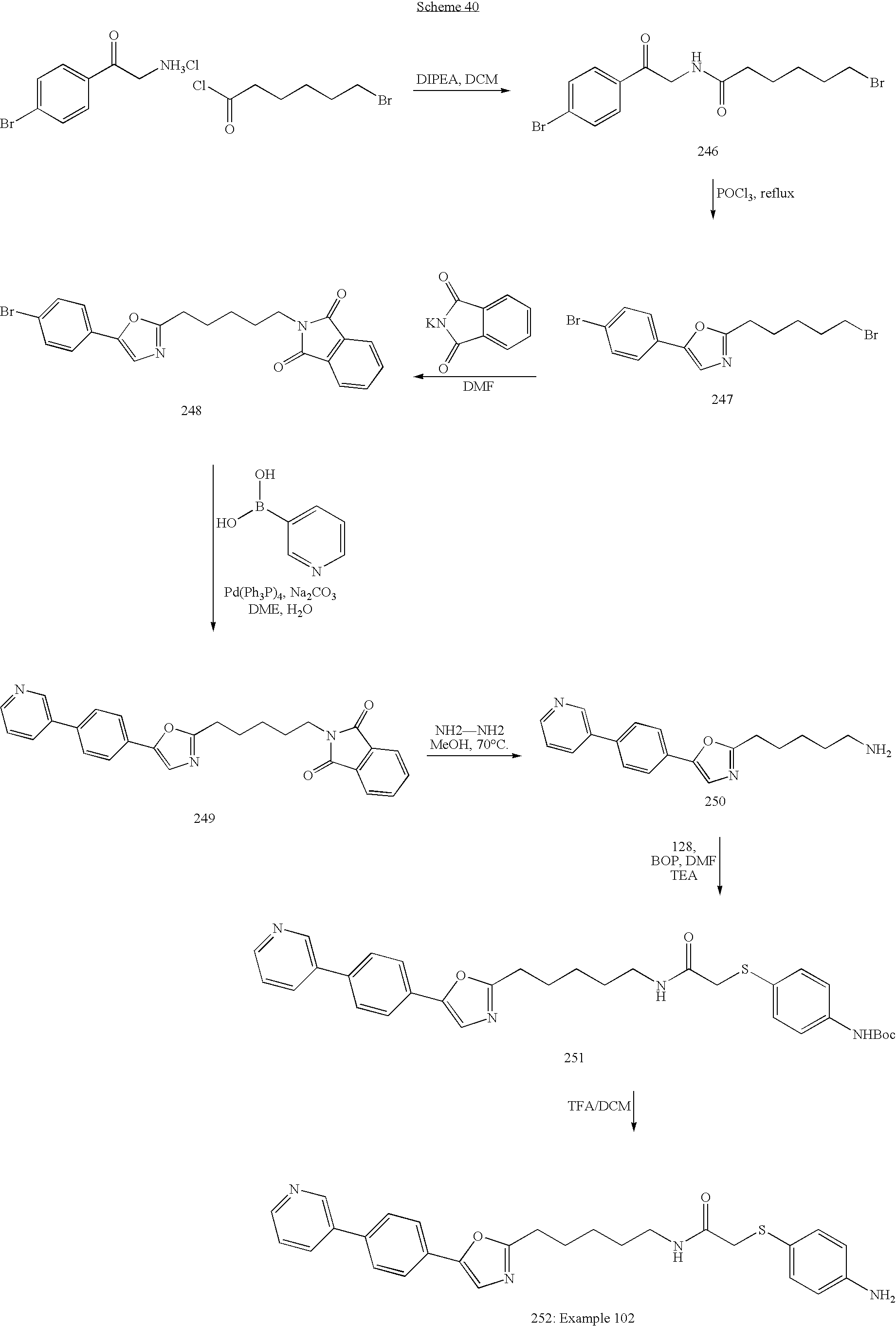

### EXAMPLE 102

### 2-(4-aminophenylthio)-N-(5-(5-(4-(pyridin-3-yl)phenyl)oxazol-2-yl)pentyl)acetamide (252)

### Step 1: 6-bromo-N-(2-(4-bromophenyl)-2-oxoethyl)hexanamide (246)

Following the same procedure as described for compound 106 (step 4, scheme 11, example 66) but substituting 6-bromohexanoyl chloride for 3-chlorocarbonylmethylsulfanyl-propionic acid methyl ester and 4-bromophenacylamine hydrochloride for amine 105 to afford 246 as a pink solid. LRMS (ESI): (calc) 389.0 (found) 390.1 (MH)⁺.

### Step 2: 2-(5-bromopentyl)-5-(4-bromophenyl)oxazole (247)

The crude compound 246 was dissolved in 20 mL of POCl₃ and stirred at 100° C. for 45 minutes. The mixture was then concentrated under reduced pressure and the residue was dissolved in ethyl acetate. The organic solution was washed with a NaHCO₃ (ss), water and brine. The organic phase was dried (Na₂SO₄), filtered and evaporated to afford 247 (530 mg, 33% for 2 steps) as a beige solid. LRMS (ESI): (calc) 371.0 (found) 372.0 (MH)⁺.

### Step 3: 2-(5-(5-(4-bromophenyl)oxazol-2-yl)pentyl)isoindoline-1,3-dione (248)

The crude compound 247 (530 mg, 1.43 mmol) was dissolved in DMF (10 mL) and potassium phthalamide (344 mg, 1.86 mmol) was added. The solution was stirred at 70° C. for 3 h then cooled at room temperature and poured into water. The product was extracted with ethyl acetate/hexanes 1:1. The combined organic phases were washed with water, 5% aqueous sodium hydroxide solution and brine. The organic phase was dried (Na₂SO₄), filtered and evaporated to afford 248 (204 mg, 33%) as white solid. LRMS (ESI): (calc) 438.1 (found) 439.2 (MH)⁺.

### Step 4: 2-(5-(5-(4-(pyridin-3-yl)phenyl)oxazol-2-yl)pentyl)isoindoline-1,3-dione (249)

Compound 248 (204 mg, 0.466 mmol) and 3-pyridineboronic acid (63 mg, 0.512 mmol) were dissolved in 10 mL of DME/water 4:1. Pd(Ph₃P)₄ (54 mg, 0.047 mmol) and sodium carbonate (100 mg, 0.932 mmol) were added and the solution was stirred at 90° C. for 1 hour. The reaction mixture was cooled at room temperature and poured into ether. The organic phase was washed with water, brine and dried (Na₂SO₄), filtered and concentrated. The residue was purified by silica gel column chromatography with 100% ethylacetate to afford 249 (122 mg, 60%) as a white solid. LRMS (ESI): (calc) 437.2 (found) 438.3 (MH)⁺.

### Step 5: 5-(5-(4-(pyridin-3-yl)phenyl)oxazol-2-yl)pentan-1-amine (250)

Following the same procedure as described for compound 139 (step 5, scheme 16, example 73a) but substituting 249 for 138 and stirring at 70° C. for 3 h to afford 250 (55 mg, 64%) as a white solid. LRMS (ESI): (calc) 307.2 (found) 308.4 (MH)⁺.

### Step 6: tert-butyl 4-(2-oxo-2-(5-(5-(4-(pyridin-3-yl)phenyl)oxazol-2-yl)pentylamino)ethylthio)phenylcarbamate (251)

Following the same procedure as described for compound 1 (step 1, scheme 1, example 1) but substituting 2-(4-(tert-butoxycarbonylamino)phenylthio)acetic acid for N-Boc-caproic acid and amine 250 for 3-phenyl aniline to afford 251 (19 mg, 19%) as a colorless oil. LRMS (ESI): (calc) 572.3 (found) 573.5 (MH)⁺.

### Step 7: 2-(4-aminophenylthio)-N-(5-(5-(4-(pyridin-3-yl)phenyl)oxazol-2-yl)pentyl)acetamide (252)

Following the same procedure as described for compound 2 (step 2, scheme 1, example 1) but substituting 251 for 1 to afford 252 (10 mg, 63%) as a white solid. (MeOD-d4) δ(ppm) ¹H: 8.85 (dd, J=2.4, 0.8 Hz, 1H), 8.53 (dd, J=5.0, 1.6 Hz, 1H), 8.14 (ddd, J=8.0, 2.4, 1.6 Hz, 1H), 7.82 (d, J=8.8 Hz, 2H), 7.76 (d, J=8.8 Hz, 2H), 7.54 (ddd, J=8.0, 5.0, 0.8 Hz, 1H), 7.47 (s, 1H), 7.19 (d, J=8.4 Hz, 2H), 6.63 (d, J=8.4 Hz, 2H), 3.34 (s, 2H), 3.14 (t, J=6.8 Hz, 2H), 2.85 (t, J=7.6 Hz, 2H), 1.84-1.76 (m, 2H), 1.50-1.43 (m, 2H), 1.35-1.27 (m, 2H). LRMS (ESI): (calc) 472.2 (found) 473.6 (MH)⁺.

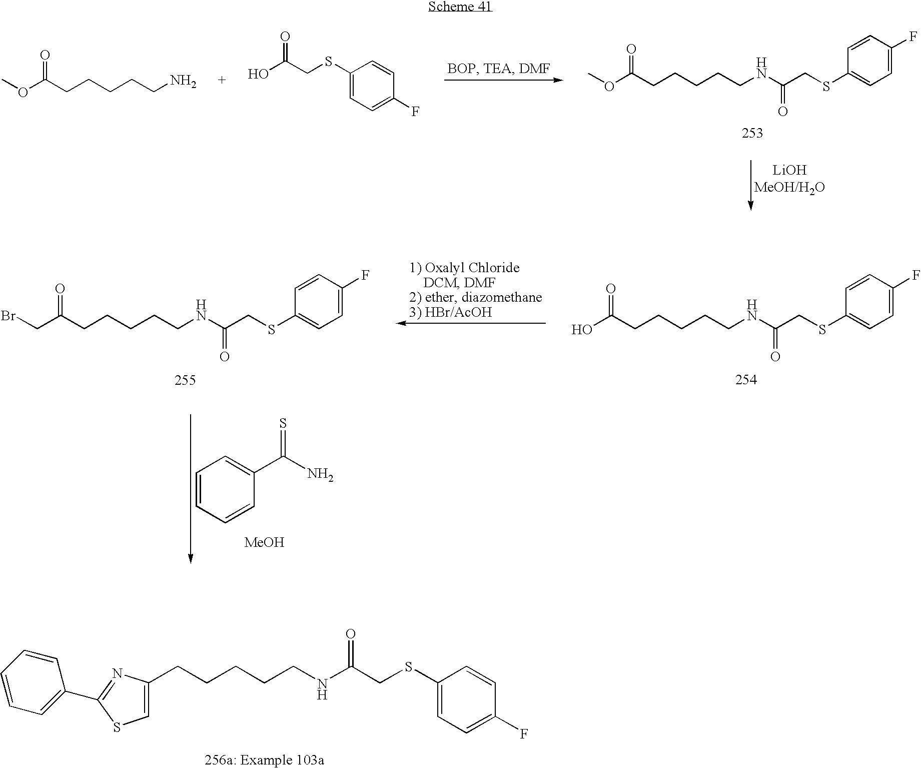

### EXAMPLE 103a

### 2-(4-fluorophenylthio)-N-(5-(2-phenylthiazol-4-yl)pentyl)acetamide (256a)

### Step 1: methyl 6-(2-(4-fluorophenylthio)acetamido)hexanoate (253)

Following the same procedure as described for compound 1 (step 1, scheme 1, example 1) but substituting 2-(4-fluorophenylthio)acetic acid for N-Boc-caproic acid and methyl 6-aminohexanoate hydrochloride for 3-phenyl aniline to afford 253 (1.24 g, quantitative yield) as a colorless oil. LRMS (ESI): (calc) 313.1 (found) 314.3 (MH)⁺.

### Step 2: 6-(2-(4-fluorophenylthio)acetamido)hexanoic acid (254)

Following the same procedure as described for compound 5 (step 5, scheme 1, example 1) but substituting 253 for methyl ester 4 and using MeOH/water 3:1 as solvent to afford 254 (1.20 g, quantitative yield) as a white solid. LRMS (ESI): (calc) 299.1 (found) 300.1 (MH)⁺.

### Step 3: N-(7-bromo-6-oxoheptyl)-2-(4-fluorophenylthio)acetamide (255)

To a solution of 254 (1.20 g, 4.0 mmol) in dichloromethane (20 mL) was added oxalyl chloride (0.45 mL, 1.3 eq) and DMF (3 drops). The reaction was stirred for 30 minutes at room temperature and then concentrated under reduced pressure. The residue was dissolved in DCM (75 mL) and an excess of diazomethane (prepared from N-nitroso-N-methylurea) was added at room temperature. After stirring 16 hours at room temperature, the solution was cooled at 0° C. and a solution of 33 wt. % of HBr in acetic acid (3.0 mL, 16.6 mmol) was added dropwise. The reaction mixture was stirred 1 h and then washed with NaHCO₃ (ss). The organic phase was dried (Na₂SO₄), filtered and evaporated. The brown oil was purified by silica gel column chromatography with ethyl acetate (50%) in hexanes to afford 255 (0.721 g, 48%) as a brown oil. LRMS (ESI): (calc) 375.0 (found) 376.0 (MH)⁺.

### Step 4: 2-(4-fluorophenylthio)-N-(5-(2-phenylthiazol-4-yl)pentyl)acetamide (256a)

To a solution of 255 (113 mg, 0.301 mmol) in MeOH (3.0 mL) was added benzothioamide (45 mg, 0.331 mmol). After stirring 1 hour at room temperature, the solution was concentrated under reduced pressure. The residue was purified by preparative HPLC with a gradient of methanol (30-90%) in water in aquasil-C₁₈ column to afford 256a (58 mg, 12%). (MeOD-d4) δ(ppm) ¹H: 7.92-7.89 (m, 2H), 7.48-7.42 (m, 5H), 7.13 (t, J=0.8 Hz, 1H), 7.08-7.04 (m, 2H), 3.52 (s, 2H), 3.15 (t, J=6.8 Hz, 2H), 2.77 (t, J=7.2 Hz, 2H), 1.76-1.68 (m, 2H), 1.50-1.43 (m, 2H), 1.33-1.26 (m, 2H). LRMS (ESI): (calc) 414.12 (found) 415.30 (MH)⁺.

### EXAMPLE 103b,c

Example 103b,c describe the preparation of compound 256b,c using the same procedures as described for compound 256a in Example 103a. Characterization data are presented in a Table 20.

TABLE 20

| Ex | Cpd | structure | Name | Characterization | Scheme |
|---|---|---|---|---|---|
| 103b | 256b | 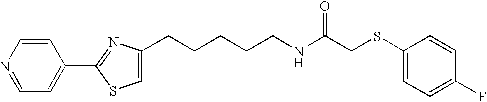 | 2-(4-fluoro- phenylthio)- N-(5-(2-(pyridin- 4-yl)thiazol-4- yl)pentyl)- acetamide | (MeOD-d4) δ(ppm) ¹H: 8.64 (d, J=1.6 Hz, 1H), 8.63 (d, J=1.6 Hz, 1H), 7.94 (d, J=1.6 Hz, 1H), 7.93 (d, J=1.6 Hz, | |
| # 1H), 7.44 (dd, J=8.8 Hz, 5.6 Hz, 2H), 7.34 (s, 1H), 7.06 (t, J=8.8 Hz, 2H), 3.52 (s, 2H), 3.15 (t, J=6.8 Hz, 2H), 2.81 (t, J=7.6 Hz, 2H), 1.77-1.70 (m, 2H), 1.50-1.43 (m, 2H), 1.33-1.25 (m, 2H) LRMS (ESI): (calc) 415.12 (found) 416.19 (MH)⁺ | 41 (Steps 1-4) Ex 103a | | | | |
| 103c | 256c | 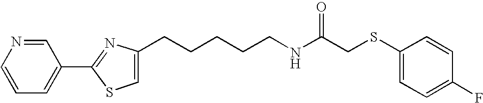 | 2-(4-fluoro- phenylthio)- N-(5-(2-(pyridin- 3-yl)thiazol-4- yl)pentyl)- acetamide | (MeOD-d4) δ(ppm) ¹H: 9.90 (d, J=2.4 Hz, 1H), 8.60 (dd, J=4.8, 1.6 Hz, 1H), 8.35-8.32 (m, 1H), 7.54 (ddd, J=8.0, 4.8, 0.4 Hz, | |
| # 1H), 7.46-7.42 (m, 2H), 7.28 (s, 1H), 7.08-7.04 (m, 2H), 3.52 (s, 2H), 3.15 (t, J=6.8 Hz, 2H), 2.80 (t, J=7.2 Hz, 2H), 1.77-1.70 (m, 2H), 1.50-1.42 (m, 2H), 1.33-1.26 (m, 2H) LRMS (ESI): (calc) 415.12 (found) 416.16 (MH)⁺ | 41 (Steps 1-4) Ex 103a | | | | |

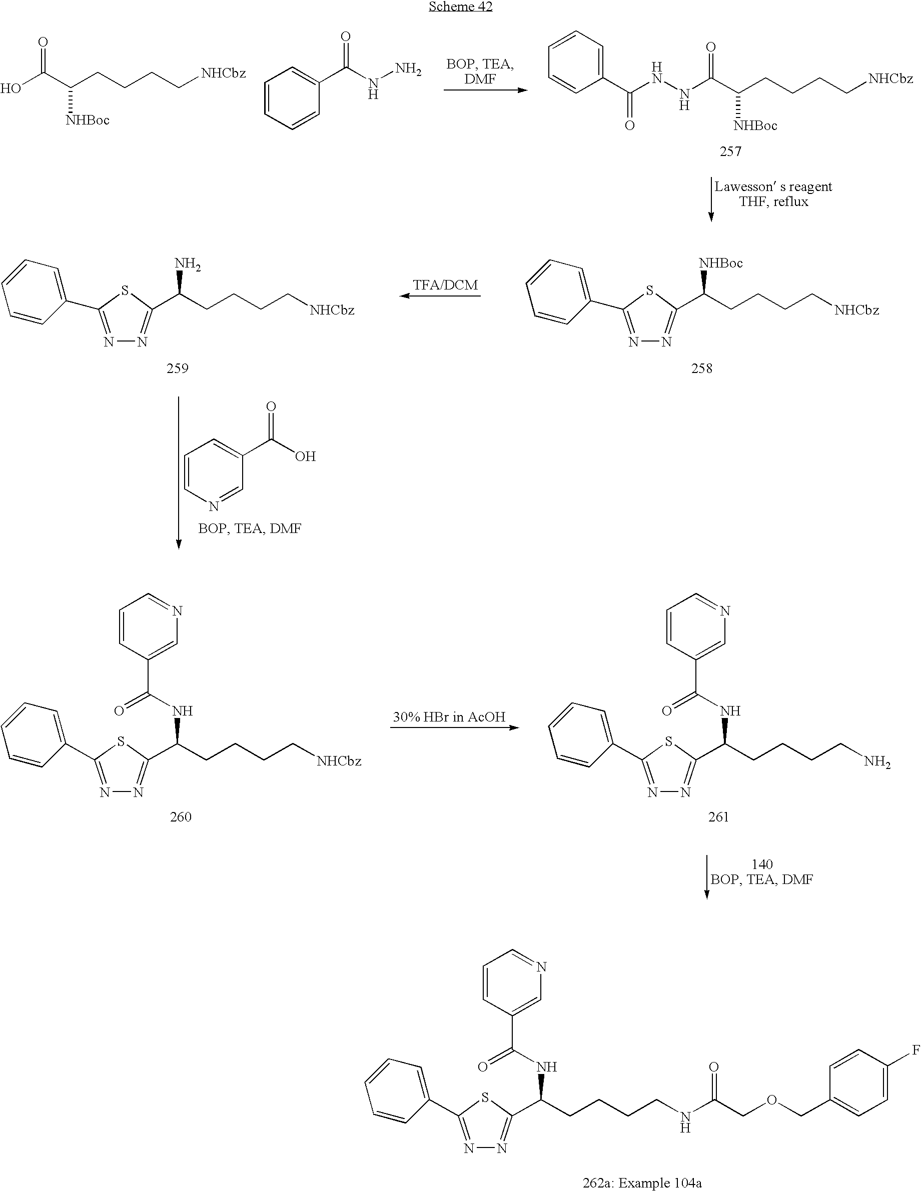

### EXAMPLE 104a

### (S)-N-(5-(2-(4-fluorobenzyloxy)acetamido)-1-(5-phenyl-1,3,4-thiadiazol-2-yl)pentyl)nicotinamide (262a)

### Step 1: (S)-benzyl 5-(t-butylcarbamate)-6-(2-benzoylhydrazinyl)-6-oxohexylcarbamate (257)

Following the same procedure as described for compound 1 (step 1, scheme 1, example 1) but substituting BOC-LYS(Z)-OH for N-Boc-caproic acid and benzhydrazide for 3-phenyl aniline to afford 257 (7.4 g, 80%) as a white solid. LRMS (ESI): (calc) 498.2 (found) 499.4 (MH)⁺.

### Step 2: (S)-benzyl 5-(t-butylcarbamate)-5-(5-phenyl-1,3,4-thiadiazol-2-yl)pentylcarbamate (258)

Following the same procedure as described for compound 176 (step 2, scheme 23, example 82) but substituting 257 for 175 to afford 258 (2.39 g, 46%) as a white solid. LRMS (ESI): (calc) 496.2 (found) 497.3 (MH)⁺.

### Step 3: (S)-benzyl 5-amino-5-(5-phenyl-1,3,4-thiadiazol-2-yl)pentylcarbamate (259)

Following the same procedure as described for compound 2 (step 2, scheme 1, example 1) but substituting 258 for 1 to afford 259 (2.39 g, 46%) as a white solid. LRMS (ESI): (calc) 496.2 (found) 497.3 (MH)⁺.

### Step 4: (S)-benzyl 5-(nicotinamido)-5-(5-phenyl-1,3,4-thiadiazol-2-yl)pentylcarbamate (260)

Following the same procedure as described for compound 1 (step 1, scheme 1, example 1) but substituting nicotinic acid for N-Boc-caproic acid and 259 for 3-phenyl aniline to afford 260 (256 mg, 92%) as a white foam. LRMS (ESI): (calc) 501.2 (found) 502.3 (MH)⁺.

### Step 5: (S)-N-(5-amino-1-(5-phenyl-1,3,4-thiadiazol-2-yl)pentyl)nicotinamide (261)

Compound 260 (256 mg, 0.511 mmol) was dissolved in 33% wt. HBr in acetic acid (3.0 mL) and stirred 1 hour. Then the reaction mixture was concentrated and the orange solid obtained was washed with 30% ethyl acetate in hexanes. The solid was dissolved in water. A solution of 5% aqueous sodium hydroxide solution was added until PH=12. The product was extracted with DCM. The combined organic phases were dried (Na₂SO₄), filtered and evaporated to afford 260 (251 mg, quantitative) as a beige solid. LRMS (ESI): (calc) 367.1 (found) 368.3 (MH)⁺.

### Step 6: (S)-N-(5-(2-(4-fluorobenzyloxy)acetamido)-1-(5-phenyl-1,3,4-thiadiazol-2-yl)pentyl)nicotinamide (262a)

Following the same procedure as described for compound 1 (step 1, scheme 1, example 1) but substituting 2-(4-fluorobenzyloxy)acetic acid for N-Boc-caproic acid and 261 for 3-phenyl aniline to afford 262a (78 mg, 27%) as a colorless oil. (MeOD-d4) δ(ppm) ¹H: 9.04 (s, 1H), 8.71 (s, 1H), 8.31 (d, J=3.6 Hz, 1H), 7.94 (s, 2H), 7.53-7.51 (m, 4H), 7.35 (d, J=5.3 Hz, 2H), 7.08-7.04 (m, 2H), 5.60 (t, J=6.1 Hz, 1H), 4.52 (s, 2H), 3.90 (s, 2H), 3.30-3.27 (m, 2H), 2.28-2.18 (m, 2H), 1.65-1.55 (m, 4H)

LRMS (ESI): (calc) 533.19 (found) 534.57 (MH)⁺.

### EXAMPLE 104b

Example 104b describe the preparation of compound 262b using the same procedures as described for compound 262a in Example 104a. Characterization data are presented in Table 21.

TABLE 21

| | | | | | Scheme |
|---|---|---|---|---|---|
| Ex | Cpd | structure | Name | Characterization | (steps) |
| 104b | 262b | 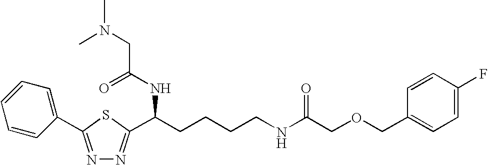 | (S)-2-(di- methylamino)- N-(5-(2-(4- fluorobenzyloxy)- acetamido)-1-(5- phenyl-1,3,4- thiadiazol-2- yl)pentyl)- acetamide | (MeOD-d4) δ(ppm) ¹H: 7.93 (d, J=7.4 Hz, 2H), 7.57-7.52 (m, 3H), 7.41-7.37 (m, 2H), 7.08 (t, | |
| # J=8.6 Hz, 2H), 5.40 (dd, J=8.8, 5.9 Hz, 1H), 4.55 (s, 2H), 3.92 (s, 2H), 3.27 (t, J=6.8 Hz, 2H), 3.06 (s, 2H), 2.31 (s, 6H), 2.12-2.02 (m, 2H), 1.65-1.42 (m, 4H) LRMS (ESI): (calc) 513.22 (found) 514.87 (MH)⁺ | 42 (1-6 (ex 104a)) | | | | |

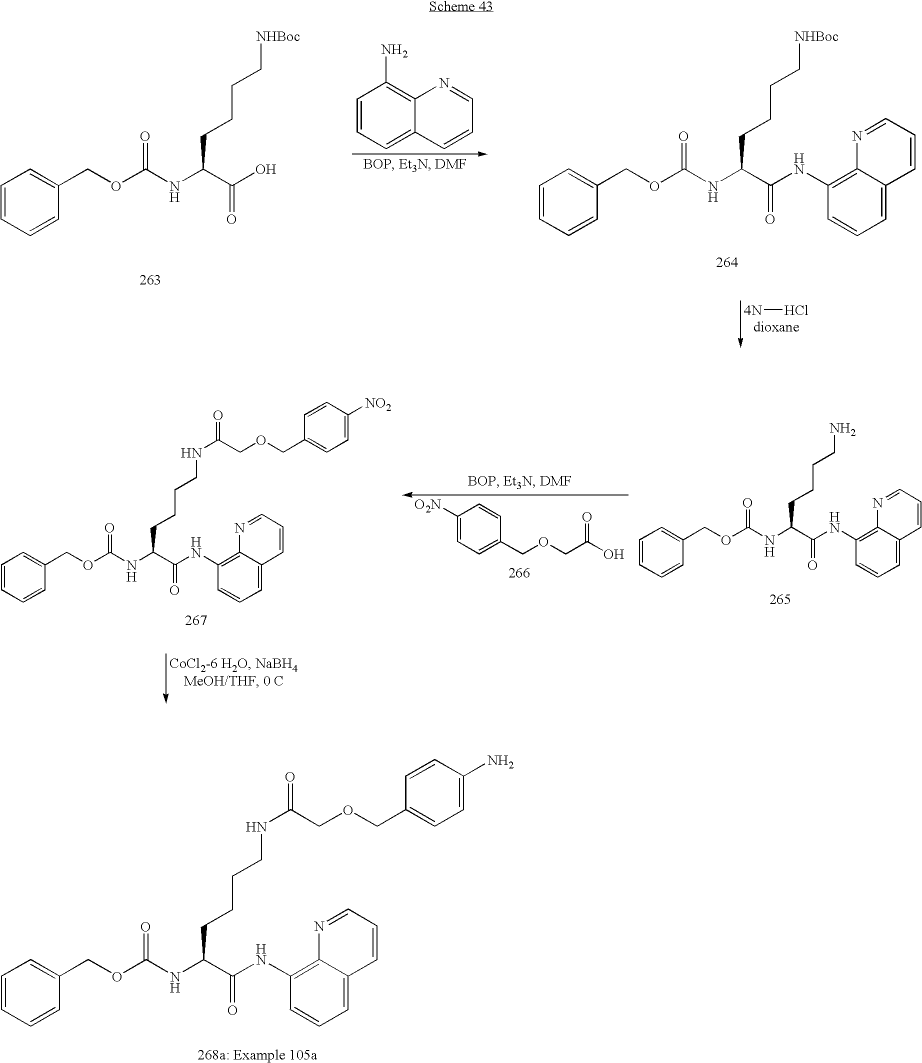

### EXAMPLE 105a

### (S)-benzyl 6-(2-(4-aminobenzyloxy)acetamido)-1-oxo-1-(quinolin-8-ylamino)hexan-2-ylcarbamate (268a)

### Step 1: (S)-benzyl 6-amino-(tert-butoxycarbonylamino)-1-oxo-1-(quinoline-8-ylamino)hexan-2-ylcarbamate (264)

Following the same procedure as described for compound 1 (scheme 1, example 1, step 1) but substituting Z-L-Lys(Boc)-OH for N-Boc-caproic acid and 8-aminoquinoline for 3-phenyl aniline to afford 264 (0.92 g, 70%) as an oil. LRMS (ESI): (calc) 506.3; (found) 507.4 (MH)⁺.

### Step 2: (S)-benzyl 6-amino-1-oxo-1-(quinolin-8-ylamino)hexan-2-ylcarbamate bis hydrochloride (265)

Compound 264 (0.3 g, 0.59 mmol) was treated with 4N—HCl in dioxane (10 mL) for 2 hours at room temperature. The mixture was then concentrated to afford 265 (0.38 g, quantitative yield) as a solid. LRMS (ESI): (calc) 406.2; (found) 406.5 (MH)⁺.

### Intermediate: 2-(4-nitrobenzyloxy)acetic acid (266)

Following the same procedure as described for compound 42 (scheme 2, example 37 step 1) but substituting (4-nitrophenyl)methanol for (4-(methylthio)phenyl)methanol and then following the same procedure as described for compound 5 (step 5, scheme 1, example 1) but substituting ethyl 2-(4-nitrobenzyloxy)acetate for methyl ester 4, to afford 266 (0.96 g, 94%). LRMS (ESI): (calc) 211.1; (found) 210.2 (MH)⁺.

### Step 3: (S)-benzyl 6-(2-(4-nitrobenzyloxy)acetamido)-1-oxo-1-(quinolin-8-ylamino)hexan-2-ylcarbamate (267)

Following the same procedure as described for compound 1 (scheme 1, example 1, step 1) but substituting acid 2-(4-nitrobenzyloxy)acetic acid for N-Boc-caproic acid and compound 265 for 3-phenyl aniline to afford 267 (40 mg, 22%). LRMS (ESI): (calc) 599.2; (found) 600.3 (MH)⁺.

### Step 4: (S)-benzyl 6-(2-(4-aminobenzyloxy)acetamido)-1-oxo-1-(quinolin-8-ylamino)hexan-2-ylcarbamate (268)

To solution of compound 267 (18 mg, 30 mmol) and cobalt chloride hexahydrate (71 mg, 0.3 mmol) in a 1:1 mixture of THF and MeOH (1 mL) at 0° C. was added sodium borohydride (11 mg, 0.3 mmol). After stirring for 35 minutes, the mixture was acidified with 3N HCl, and then basified with concentrated ammonium hydroxide. Extraction with DCM, the organic layers were dried (Na₂SO₄), filtered, and concentrated. The residue was purified by silica gel column chromatography with a 5% MeOH in DCM to afford 268 (7 mg, 41%). (MeOD-d4) δ(ppm) ¹H: 8.75 (dd, J=4.3, 1.3 Hz, 1H), 8.64 (d, J=7.4 Hz, 1H), 8.29 (dd, J=8.1, 1.3 Hz, 1H), 7.62 (d, J=8.4 Hz, 1H), 7.56-7.50 (m, 2H), 7.40-7.20 (m, 5H), 7.07 (d, J=8.4 Hz, 2H), 6.68 (d, J=8.2 Hz, 2H), 5.21 and 5.10 (AB doublet, J=12.4 Hz, 2H), 4.40 (s, 2H), 4.36-4.32 (m, 1H), 3.84 (s, 2H), 3.23 (t, J=6.6 Hz, 2H), 2.12-1.98, (m, 1H), 1.90-1.77 (m, 1H), 1.70-1.43 (m, 4H); LRMS (ESI): (calc) 569.3 (found) 570.4 (MH)⁺.

### EXAMPLE 105b

Example 105b describe the preparation of compound 268b using the same procedures as described for compound 268a in Example 105a. Characterization data are presented in Table 22.

TABLE 22

| | | | | | Scheme |
|---|---|---|---|---|---|
| Ex | Cpd | structure | Name | Characterization | (steps) |
| 105b | 268b | 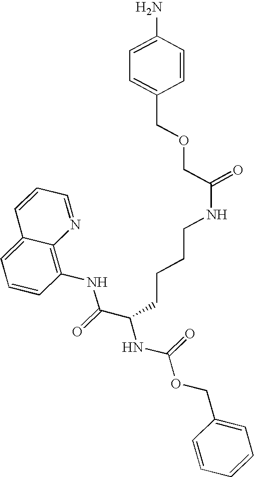 | (R)-benzyl 6-(2-(4- aminobenzyloxy)- acetamido)-1-oxo-1- (quinolin-8-ylamino)- hexan-2-ylcarbamate | (MeOD-d4) δ(ppm) ¹H: 8.76-8.74 (m, 1H), 8.63 (d, J=7.6 Hz, 1H), 8.30-8.27 (m, 1H), 7.61 (d, J=8.3 Hz, 1H), 7.55-7.50 (m, 2H), 7.40-7.20 (m, 5H), 7.07 | |
| # (d, J=8.2 Hz, 2H), 6.68 (d, J=8.2 Hz, 2H), 5.21 and 5.10 (AB doublet, J=12.5 Hz, 2H), 4.40 (s, 2H), 4.36-4.32 (m, 1H), 3.84 (s, 2H), 3.23 (t, J=6.3 Hz, 2H), 2.12-1.95, (m, 1H), 1.90-1.77 (m, 1H), 1.65-1.43 (m, 4H). LRMS (ESI): (calc) 569.3 (found) 570.97 (MH)⁺. | 43 | | | | |

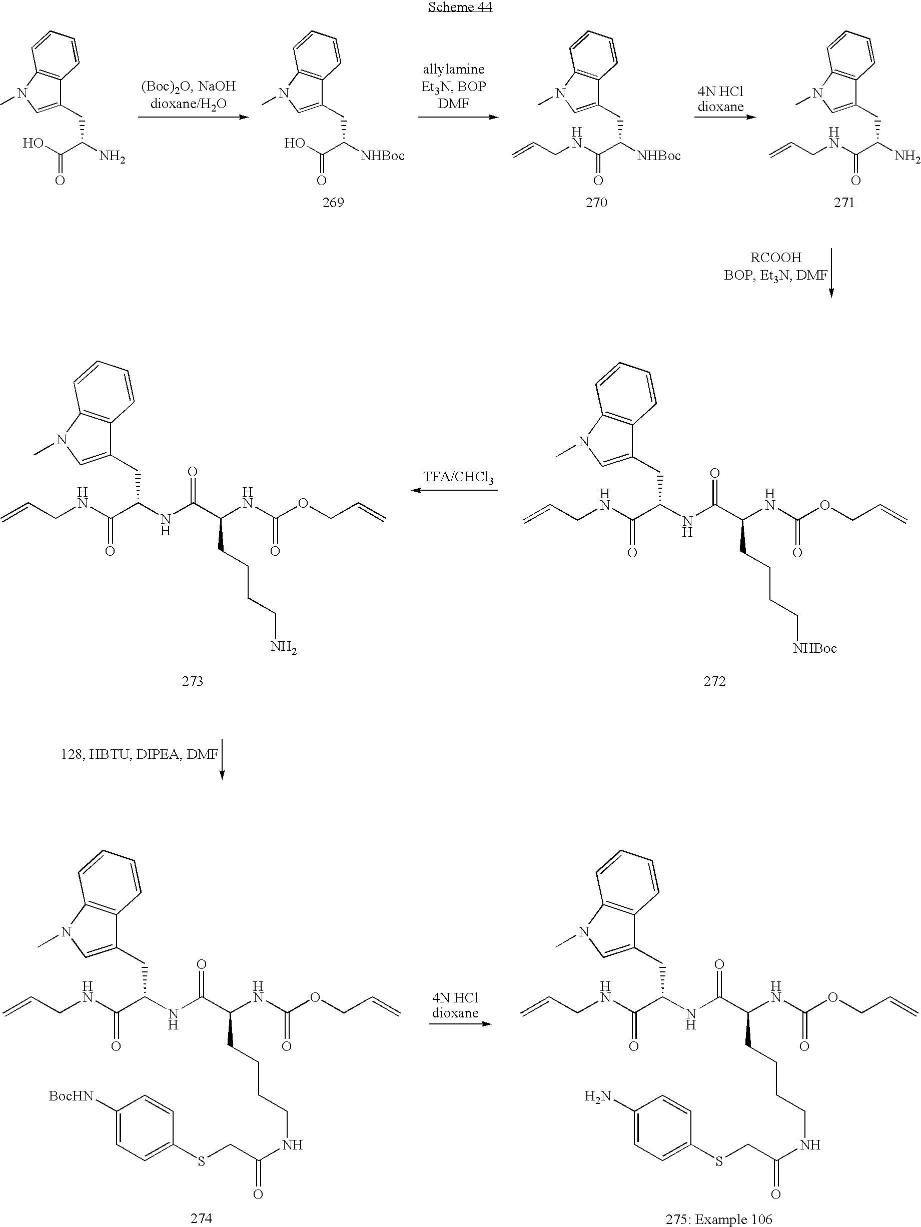

### EXAMPLE 106

### allyl(S)-1-((S)-1-(allylamino)-3-(1-methyl-1H-indol-3-yl)-1-oxopropan-2-ylamino)-6-(2-(4-aminophenylthio)acetamido)-1-oxohexan-2-ylcarbamate (275)

### Step 1: (S)-2-(tert-butoxycarbonylamino)-3-(1-methyl-1H-indol-3-yl)propanoic acid (269)

To a solution of 1-methyl-L-trp-OH (1 g, 4.6 mmol) and di-tert-butyl dicarbonate (1.5 g, 6.9 mmol) in a 1:1 mixture of water/dioxane (40 mL) at 0° C. was added sodium hydroxide (0.4 g, 10 mmol). The resulting mixture was stirred at 0° C. for 2 hours, then diluted with ethyl ether, acidified with 3N HCl and extracted with ethyl acetate. The organic layer was extracted from brine and dried (Na₂SO₄), filtered and concentrated to afford 275 (1.4 g, 96%). LRMS (ESI): (calc) 318.2 (found) 317.3 (MH)⁺.

### Step 2: (S)-tert-butyl 1-(allylamino)-3-(1-methyl-1H-indol-3-yl)-1-oxopropan-2-ylcarbamate (270)

Following the same procedure as described for compound 1 (scheme 1, example 1) but substituting compound 269 for N-Boc-caproic acid and allylamine for 3-phenyl aniline to afford 270 (1.6 g, quantitavive). LRMS (ESI): (calc) 357.2; (found) 357.3 (MH)⁺.

### Step 3: (S)-N-allyl-2-amino-3-(1-methyl-1H-indol-3-yl)propanamide (271)

Following the same procedure as described for compound 265 (scheme 43, example 105a, step 2) but substituting 270 for 264 to afford 271 (1.3 g, quant.). LRMS (ESI): (calc) 257.2; (found) 257.3 (MH)⁺.

### Step 4: allyl(S)-1-((S)-1-(allylamino)-3-(1-methyl-1H-indol-3-yl)-1-oxopropan-2-ylamino)-6-(tert-butoxycarbonylamino)-1-oxohexan-2-ylcarbamate (S)-2-amino-3-(1-methyl-1H-indol-3-yl)propanoate (272)

Following the same procedure as described for compound 1 (scheme 1, example 1) but substituting Aloc-L-Lys(Boc)-OH-DCHA for N-Boc-caproic acid and compound 271 for 3-phenyl aniline to afford 272 (1.6 g, quantitative yield). LRMS (ESI): (calc) 569.3; (found) 570.3 (MH)⁺.

### Step 5: allyl(S)-1-((S)-1-(allylamino)-3-(1-methyl-1H-indol-3-yl)-1-oxopropan-2-ylamino)-6-amino-1-oxohexan-2-ylcarbamate (S)-2-amino-3-(1-methyl-1H-indol-3-yl)propanoate (273)

Following the same procedure as described for compound 9 (scheme 1, example 4, step 5) but substituting compound 272 for compound 7a and using chloroform as a solvent to afford 273 (45 mg, 74%). LRMS (ESI): (calc) 469.3; (found) 470.4 (MH)⁺.

### Step 6: allyl(S)-1-((S)-1-(allylamino)-3-(1-methyl-1H-indol-3-yl)-1-oxopropan-2-ylamino)-6-(2-(4-tert-butyl thiophenylcarbamate)acetamido)-1-oxohexan-2-ylcarbamate (274)

To a stirred solution of 128 (33 mg, 0.12 mmol) and DIPEA (37 mg, 0.29 mmol) in DMF (1 mL) was added HBTU (40 mg, 0.11 mmol). After 15 minutes 273 (45 mg, 0.1 mmol) was added at room temperature for 3 h. The mixture was diluted with ethyl acetate, extracted from brine, dried (Na₂SO₄), filtered and concentrated. The residue was purified by silica gel column chromatography with gradient of MeOH (0-5%) in DCM to afford 274 (25 mg, 35%). LRMS (ESI): (calc) 734.4; (found) 735.4 (MH)⁺.

### Step 7: allyl(S)-1-((S)-1-(allylamino)-3-(1-methyl-1H-indol-3-yl)-1-oxopropan-2-ylamino)-6-(2-(4-aminophenylthio)acetamido)-1-oxohexan-2-ylcarbamate (275)

Following the same procedure as described for compound 265 (scheme 43, example 105a, step 2) but substituting compound 274 for compound 264 to afford 275 (5 mg, 26%). (MeOD-d4) δ(ppm) ¹H, 7.57 (d, J=7.9 Hz, 1H), 7.30 (d, J=7.8 Hz, 1H), 7.20 (dd, J=6.6, 1.5 Hz, 2H), 7.15 (t, J=7.0 Hz, 1H), 7.04 (t, J=7.4 Hz, 1H), 7.01 (s, 1H), 6.62 (dd, J=6.5, 1.8 Hz, 2H), 5.98-5.82 (m, 1H), 5.73-5.60 (m, 1H), 5.28 (d, J=17.0 Hz, 1H), 5.16 (d, 10.4 Hz, 1H), 5.03-4.95 (m, 2H), 4.62 (t, J=6.9 Hz, 1H), 4.58-4.40 (m, 2H), 4.0-3.90 (m,1H), 3.74 (s, 3H), 3.73-3.60 (m, 2H), 3.34 (s, 2H), 3.25 and 3.16 (AB doublet, J=14.6, 6.9 Hz, 2H), 3.10-2.97 (m, 2H), 1.60-1.40 (m, 2H), 1.40-1.20 (m, 2H), 1.22-1.0 (m, 2H).

LRMS (ESI): (calc) 634.3 (found) 636.1 (MH)⁺.

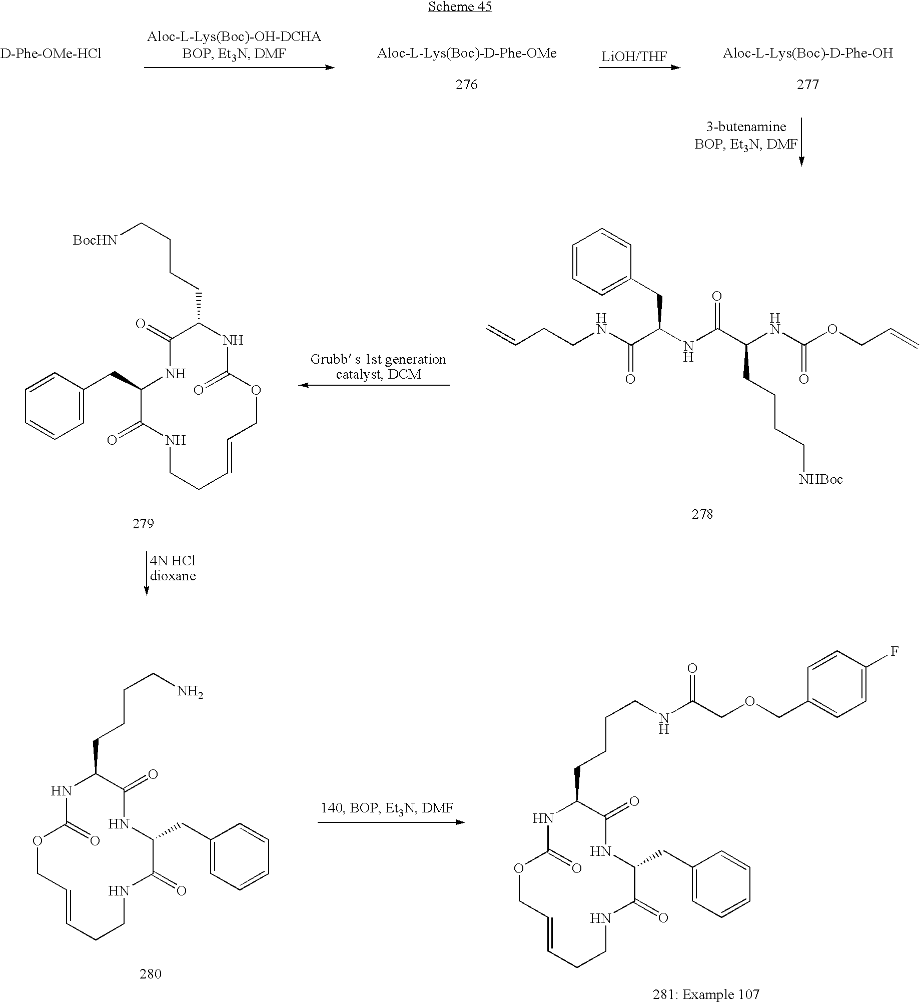

### EXAMPLE 107

### N-(4-((4S,7R,E)-7-benzyl-2,5,8-trioxo-1-oxa-3,6,9-triazacyclotetradec-12-en-4-yl)butyl)-2-(4-fluorobenzyloxy)acetamide (281)

### Step 1: Aloc-L-Lys(Boc)-D-Phe-OMe (276)

Following the same procedure as described for compound 1 (scheme 1, example 1, step 1) but substituting Aloc-L-Lys(Boc)-OH-DHCA for N-Boc-caproic acid and D-Phe-OMe-HCl for 3-phenyl aniline to afford 276 (0.12 g, 51%). LRMS (ESI): (calc) 491.3; (found) 492.6 (MH)⁺.

### Step 2: Aloc-L-Lys(Boc)-D-Phe-OH (277)

Following the same procedure as described for compound 5 (scheme 1, example 1, step 5) but substituting 276 for methyl ester 4, to afford 277 (0.21 g, 88%). LRMS (ESI): (calc) 477.3; (found) 476.6 (MH)⁺.

### Step 3: allyl(S)-6-tert-butoxycarbonylamino-1-((R)-1-(but-3-enylamino)-1-oxo-3-phenylpropan-2-ylamino)-1-oxohexan-2-ylcarbamate (278)

Following the same procedure as described for compound 1 (scheme 1, example 1, step 1) but substituting compound 277 for N-Boc-caproic acid and 3-butenamine for 3-phenyl aniline to afford 278 (0.16 g, 70%). LRMS (ESI): (calc) 530.3; (found) 531.3 (MH)⁺.

### Step 4: (4S,7R,E)-4-(4-(tert-butoxycarbonylamino)-butyl)-7-benzyl-1-oxa-3,6,9-triazacyclotetradec-12-ene-2,5,8-trione (279)

To a stirred solution of compound 278 (47 mg, 0.09 mmol) in DCM was added Grubb's 1^{st} generation catalyst (11 mg, 0.01 mmol). The mixture was stirred over night, diluted with methanol and treated with activated charcoal for 1 h then silica gel was added and the suspension was concentrated. The residue was purified by silica gel column chromatography with a gradient of ethyl acetate (25-100%) in hexanes to afford 279 (15 mg, 33%). LRMS (ESI): (calc) 502.3; (found) 503.1 (MH)⁺.

### Step 5: (4S,7R,E)-4-(4-aminobutyl)-7-benzyl-1-oxa-3,6,9-triazacyclotetradec-12-ene-2,5,8-trione (280)

Following the same procedure as described for compound 265 (scheme 43, example 105a, step 2) but substituting 279 for 264 to afford 280 (25 mg, quant.). LRMS (ESI): (calc) 402.2; (found) 403.1 (MH)⁺.

### Step 6: N-(4-((4S,7R,E)-7-benzyl-2,5,8-trioxo-1-oxa-3,6,9-triazacyclotetradec-12-en-4-yl)butyl)-2-(4-fluorobenzyloxy)acetamide (281)

Following the same procedure as described for compound 1 (scheme 1, example 1, step 1) but substituting 140 for N-Boc-caproic acid and 280 for 3-phenyl aniline to afford 281 (4 mg, 11%). (MeOD-d4) δ(ppm) ¹H: 7.44-7.38 (m, 2H), 7.29-7.17 (m, 5H), 7.12-7.06 (m, 2H), 5.80-5.68 (m, 1H), 5.50-5.40 (m, 1H), 5.01-4.62 (m, 3H), 4.62-4.52 (m, 2H), 4.28-4.19 (m, 1H), 4.00-3.79 (m, 3H), 3.74-360 (m, 1H), 3.42-3.34 (m, 1H), 3.22-3.02 (m, 3H), 2.88-2.72 (m, 2H), 2.40-2.26 (m, 1H), 2.10-2.02 (m, 1H), 1.44-0.82 (m, 8H). LRMS (ESI): (calc) 568.3 (found) 569.7 (MH)⁺.

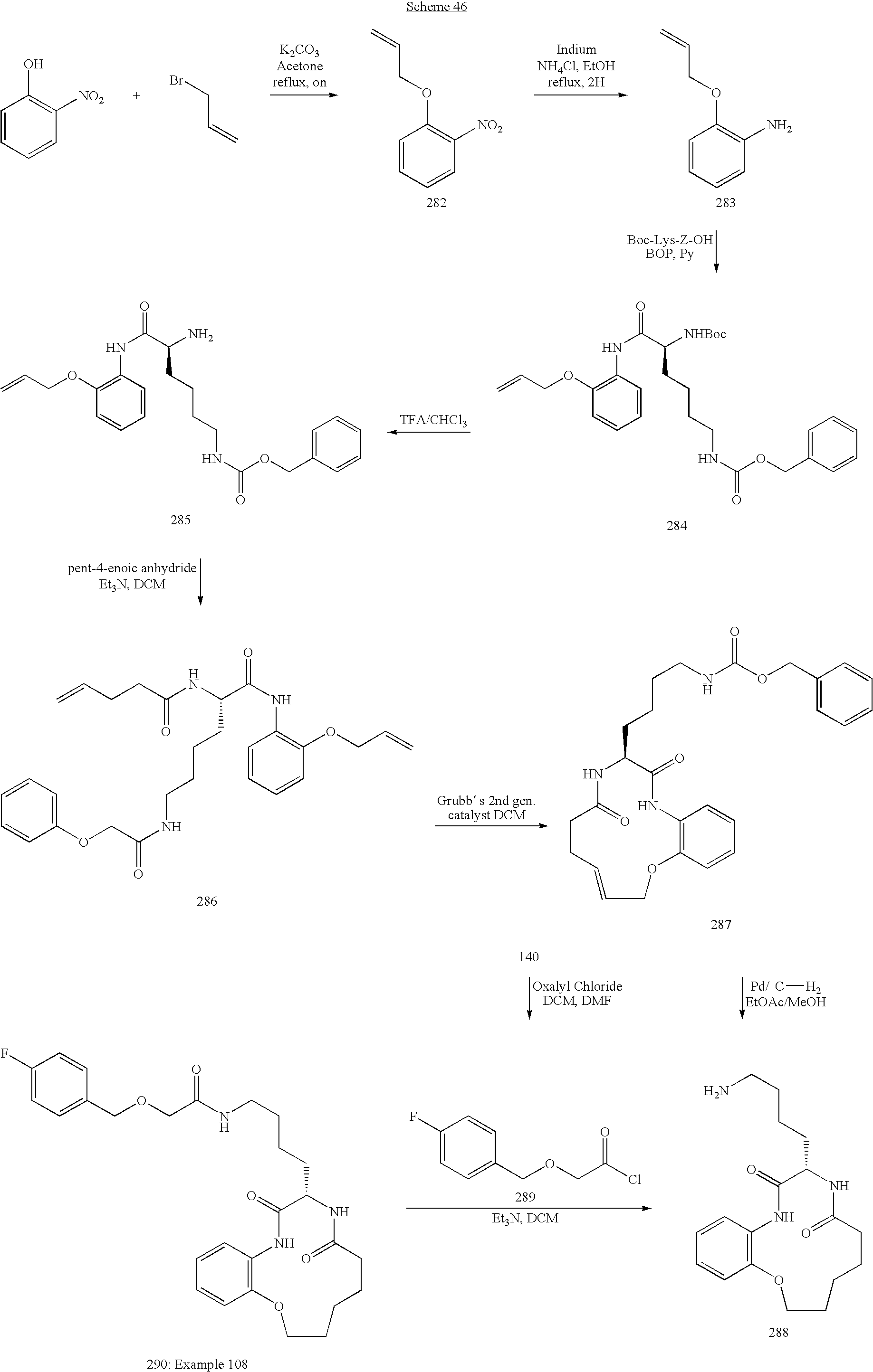

### EXAMPLE 108

### (S)-N-(4-(2,5-dioxo-1,2,3,4,5,6,7,8,9,10-decahydrobenzo[b][1,4,7]oxadiazacyclotridecin-3-yl)butyl)-2-(4-fluorobenzyloxy)acetamide (288)

### Step 1: 1-(allyloxy)-2-nitrobenzene (282)

Following the same procedure as described for compound 171 (scheme 22, example 81a, step 1) but substituting 2-nitrophenol for 5-amino-1,3,4-thiadiazole-2-thiol and allyl bromide for 2-(3-bromopropyl)isoindoline-1,3-dione to afford 282 (6.0 g, 95%) as a yellow oil. LRMS (ESI): (calc) 176.1 (found) 177.2 (MH)⁺.

### Step 2: 2-(allyloxy)aniline (283)

To a stirred suspension of compound 282 (3.0 g, 17 mmol) and ammonium chloride saturated solution (8 mL) in refluxing ethanol (35 mL) was added indium (11.8 g, 102 mmol). The mixture was stirred 2 hours, filtered, concentrated, diluted with ethyl acetate, and extracted with 3N HCl solution. The aqueous layer was basified with 10% NaOH solution and extracted with ethyl acetate. The organics layer was extracted from brine, dried (Na₂SO₄), treated with activated charcoal, filtered, and concentrated to afford 283 (1.1 g, 44%) as a black oil. LRMS (ESI): (calc) 149.1 (found) 149.9 (MH)⁺.

### Step 3: (S)-benzyl 6-(2-(allyloxy)phenylamino)-5-tert-butyl carbamate-6-oxohexylcarbamate (284)

Following the same procedure as described for compound 1 (scheme 1, example 1, step 1) but substituting Boc-L-Lys-Z-OH for N-Boc-caproic acid, 283 for 3-phenyl aniline and pyridine for triethylamine-DMF to afford 284 (1.24 g, 91%). LRMS (ESI): (calc) 511.27; (found) 512.7 (MH)⁺.

### Step 4: (S)-benzyl 6-(2-(allyloxy)phenylamino)-5-amino-6-oxohexylcarbamate (285)

Following the same procedure as described for compound 9 (scheme 1, example 4, step 5) but substituting compound 284 for compound 7a and chloroform for DCM to afford 285 (1.1 g, quantitative yield). LRMS (ESI): (calc) 411.2; (found) 412.3 (MH)⁺.

### Step 5: (S)-benzyl 6-(2-(allyloxy)phenylamino)-6-oxo-5-pent-4-enamidohexylcarbamate (286)

Following the same procedure as described for compound 54 (scheme 2, example 47, step 1) but substituting pent-4-enoic anhydride for 3-bromopropanoyl chloride and DCM for THF to afford 286 (0.23 g, 54%) as white solid. LRMS (ESI): (calc) 493.3; (found) 494.1 (MH)⁺.

### Step 6: (S,E)-benzyl 4-(2,5-dioxo-1,2,3,4,5,6,7,10-octahydrobenzo[b][1,4,7]oxadiazacyclotridecin-3-yl)butylcarbamate (287)

Following the same procedure as described for compound 279 (scheme 45, example 107, step 4) but substituting compound 286 for compound 278 and Grubb's 2^{nd} generation catalyst for Grubb's 1st generation catalyst to afford 287 (0.065 g, 30%). LRMS (ESI): (calc) 465.2; (found) 466.6 (MH)⁺.

### Step 7: (S)-3-(4-aminobutyl)-3,4,7,8,9,10-hexahydrobenzo[b][1,4,7]oxadiazacyclotridecine-2,5(1H,6H)-dione (288)

Following the same procedure as described for compound 74 (scheme 5, example 55) but substituting 287 for 73 to afford 288 (0.055 g, quant.). LRMS (ESI): (calc) 333.2; (found) 334.4 (MH)⁺.

### Intermediate: 2-(4-fluorobenzyloxy)acetyl chloride (289)

Following the same procedure as described for compound 255 (scheme 41, example 103a, step 3-1)) but substituting 140 for 254 to afford 289. The product was used without further purification.

### Step 8: (S)-N-(4-(2,5-dioxo-1,2,3,4,5,6,7,8,9,10-decahydrobenzo[b][1,4,7]oxadiazacyclotridecin-3-yl)butyl)-2-(4-fluorobenzyloxy)acetamide (290)

Following the same procedure as described for compound 3a (scheme 1, example 1) but substituting 289 for chloroacetyl chloride, compound 288 for amine compound 2 and DCM for THF to afford 290 (0.02 g, 44%). (MeOD-d4) δ(ppm) ¹H: 8.12 (dd, J=8.0, 1.5 Hz, 1H), 7.42-7.38 (m, 2H), 7.1-7.0 (m, 3H), 6.99-6.89 (m, 2H), 4.56 (s, 2H), 4.42-4.38 (m, 1H), 4.35-4.30 (m, 1H), 3.93 (s, 2H), 3.83 (td, J=9.4, 3.6 Hz, 1H), 3.28-3.23 (m, 2H), 2.6-2.5 (m, 1H), 2.29-2.14 (m, 1H), 2.05-1.89 (m, 2H), 1.88-1.39 (m, 10H). LRMS (ESI): (calc) 499.3 (found) 500.6 (MH)⁺.

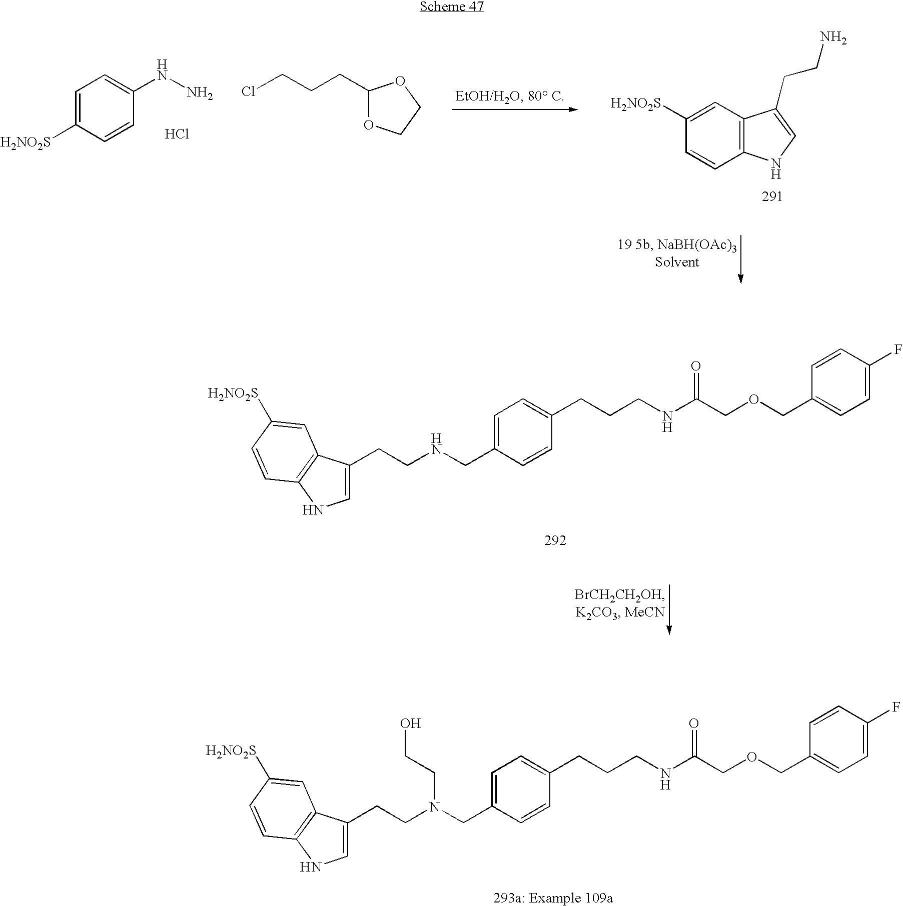

### EXAMPLE 109a

### 2-(4-fluorobenzyloxy)-N-(3-(4-((2-(5-sulfamoyl-1H-indol-3-yl)ethylamino)methyl)phenyl)propyl)acetamide (293a)

### Step 1: 3-(2-aminoethyl)-1H-indole-5-sulfonamide (291)

To a stirred solution of 4-hydrazinylbenzenesulfonamide hydrochloride (740 mg, 3.32 mmol) in a 5: 1 ethanol-water mixture (18 mL), was added 2-(3-chloropropyl)-1,3-dioxolane (500 mg, 3.32 mmol). The solution was heated to 80° C. and stirred for 2 h. The solvent was removed via rotary evaporation. The residue was purified with silica gel column chromatography employing a 20:48:2 methanol:dichloromethane:ammonium hydroxide moving to 33:63:2 methanol:dichloromethane:ammonium solvent gradient to afford 291 (400 mg, 51%) as a red solid.

(DMSO-d6) δ(ppm): 11.56 (s, 1H), 8.08 (d, J=1.6 Hz, 1H), 7.58-7.55 (m, 1H), 7.51-7.48 (m, 1H), 7.42 (d, J=2.3 Hz, 1H), 7.13 (s, 2H), 3.15 (d, J=5.1 Hz, 2H), 3.05 (s, 4H).

### Step 2: 3-(2-(4-(3-(2-(4-fluorobenzyloxy)acetamido)propyl)benzylamino)ethyl)-1H-indol-5-yl sulfite (292)

Following the same procedure as described for compound 196b (step 5, scheme 27, example 87b) but substituting 291 for tryptamine to afford 292 (60 mg, 17%) as a colourless oil.

(MeOD-d4) δ(ppm): 8.17 (d, J=1.6 Hz 1H), 7.66 (dd, J=8.6, 1.8 Hz, 1H), 7.45 (d, J=8.6 Hz, 1H), 7.38 (dd, J=8.6, 5.5 Hz, 2H),-7.20-7.04 (m, 7H), 4.53 (s, 2H), 3.90 (s, 2H), 3.74 (s, 2H), 3.23 (t, J=7.2 Hz, 2H), 3.01-2.89 (m, 4H), 2.58 (t, J=7.6 Hz, 2H), 1.79 (quintet, J=7.4 Hz, 2H).

### Step 3: 2-(4-fluorobenzyloxy)-N-(3-(4-((2-(5-sulfamoyl-1H-indol-3-yl)ethylamino)methyl)phenyl)propyl)acetamide (293a)

Following the same procedure as described for compound 197b (step 1, scheme 27, example 88b) but substituting 292 for 196b to afford 293a (15 mg, 31%) as a colourless oil.

(MeOD-d4) δ(ppm): 8.12 (d, J=1.8 Hz, 1H), 7.63 (dd, J=8.6, 1.8 Hz, 1H), 7.45-7.37 (m, 3H), 7.23 (d, J=8.0 Hz, 2H), 7.16-7.04 (m, 5H), 4.55 (s, 2H), 3.91 (s, 2H), 3.71 (s, 2H), 3.62 (t, J=6.2 Hz, 2H), 3.24 (t, J=7.0 Hz, 2H), 2.96 (t, J=6.8 Hz, 2H), 2.85-2.80 (m, 2H), 2.73 (t, J=6.3 Hz, 2H), 2.60 (t, J=7.4 Hz, 2H), 1.81 (quintet, J=7.4 Hz, 2H).

### EXAMPLE 109b,c

Example 109b describes the preparation of compound 293b, using the same procedures as described for compound 293a in Example 109a. Characterization data are presented in a Table 23.

TABLE 23

| | | | | | Scheme |
|---|---|---|---|---|---|
| Ex | Cpd | structure | Name | Characterization | (steps) |
| 109b | 293b | 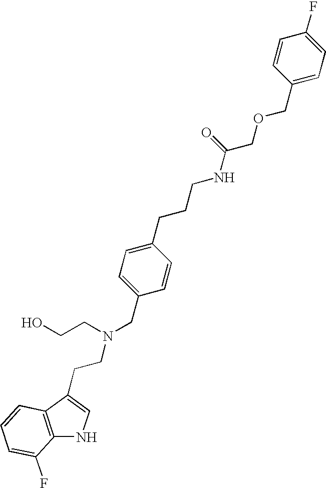 | N-(3-(4-(((2-(7- fluoro-1H-indol-3- yl)ethyl)(2- hydroxyethyl)amino)- methyl)phenyl)pro- pyl)-2-(4- fluorobenzyloxy)- acetamide | (MeOD-d4) δ(ppm) 1H: 7.38 (dd, J=8.8, 5.8 Hz, 2H), 7.24 (d, J=8.2 Hz, 2H), 7.16-7.02 (m, 6H), 6.88-6.83 (m, 1H), 6.79-6.73 (m, 1H), 4.54 (s, 2H), 3.91 (s, 2H), | |
| # 3.69 (s, 2H), 3.62 (t, J=6.5 Hz, 2H), 3.24 (t, J=7.0 Hz, 2H), 2.92-2.88 (m, 2H), 2.80-2.76 (m, 2H), 2.72 (t, J=6.3 Hz, 2H), 2.60 (t, J=7.6 Hz, 2H), 1.81 (quintet, J=7.2 Hz, 2H). LRMS (ESI): (calc.) 535.3 (found) 536.3 (MH)+ | 47 | | | | |
| 109c | 293c | 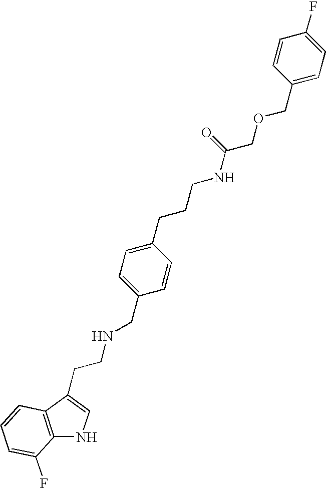 | N-(3-(4-((2-(7- fluoro-1H-indol-3- yl)ethylamino)- methyl)phenyl)propyl)- 2-(4-fluoro- benzyloxy)acetamide | (MeOD-d4) δ(ppm) 1H: 7.36 (dd, J=8.4, | |
| # 5.5 Hz, 2H), 7.28 (d, J=7.8 Hz, 1H), 7.18 (d, J=8.0 Hz, 2H), 7.13-7.02 (m, 5H), 6.94-6.88 (m, 1H), 6.83-6.77 (m, 1H), 4.51 (s, 2H), 3.90 (s, 2H), 3.77 (s, 2H), 3.22 (t, J=7.0 Hz, 2H), 3.00-2.90 (m, 4H), 2.57 (t, J=7.4 Hz, 2H), 1.78 (quintet, J=7.4 Hz, 2H). LRMS (ESI): (calc.) 491.2 (found) 492.3 (MH)+ | 47 (step 1-2) | | | | |

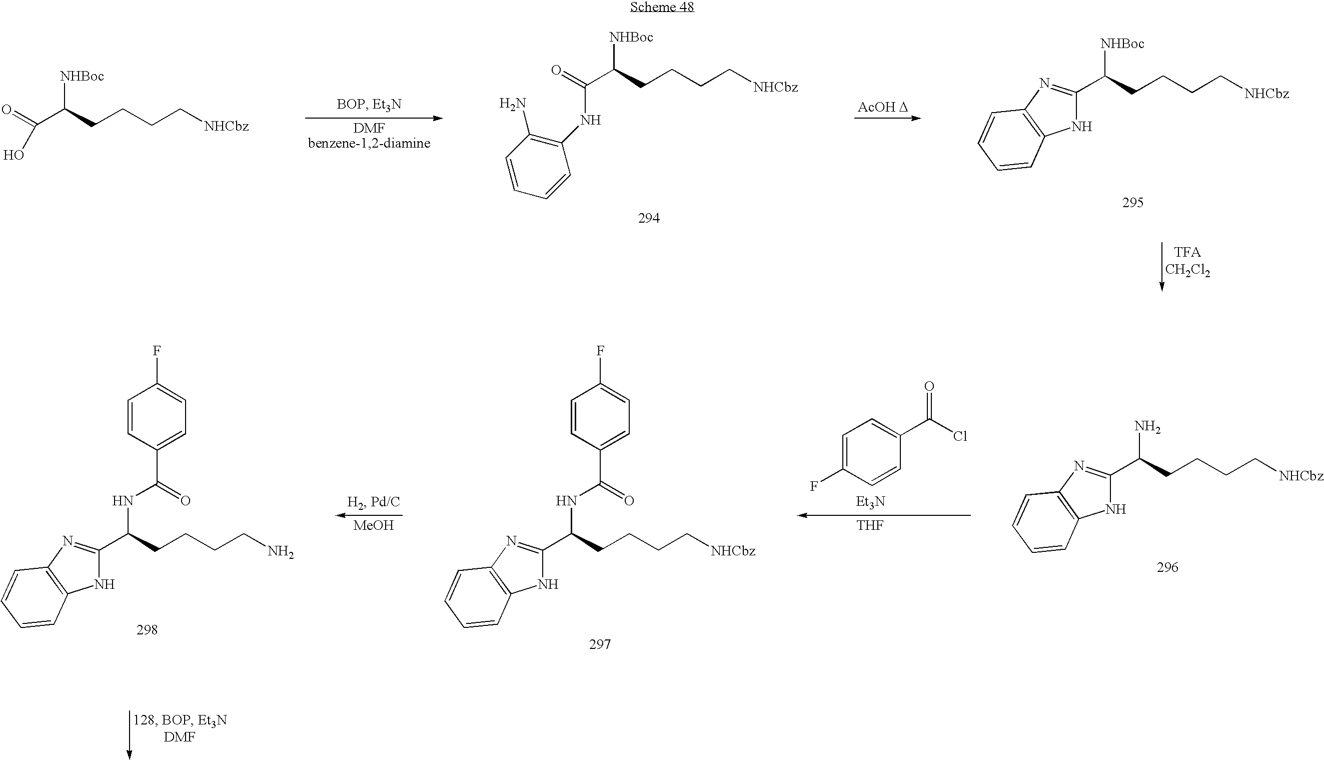

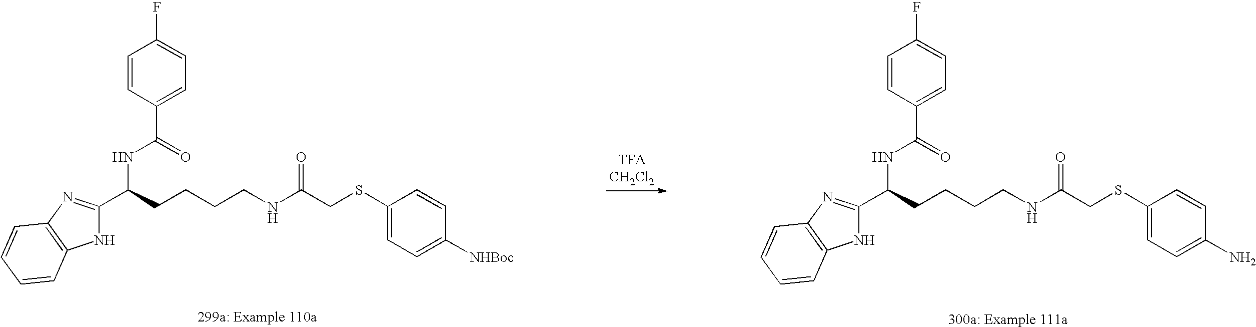

### EXAMPLE 110a

### (S)-tert-butyl 4-(2-(5-(1H-benzo[d]imidazol-2-yl)-5-(4-fluorobenzamido)pentylamino)-2-oxoethylthio)phenylcarbamate (299a)

### Step 1 (S)-6-(benzyloxycarbonylamino)-2-(tert-butoxycarbonylamino)hexanoic-2-aminobenzamide (294)

Following the same procedure as described for compound 1 (step 1, scheme 1, example 1) but substituting acid (S)-6-(benzyloxycarbonylamino)-2-(tert-butoxycarbonylamino)hexanoic acid and benzene-1,2-diamine for N-Boc-caproic acid and 3-phenyl aniline, respectively, to afford 294 (1.32 g, 99%) as a yellow oil. LRMS (ESI): (calc.) 470.2; (found) 471 (MH)⁺.

### Step 2: (S)-benzyl 5-(tert-butoxycarbonylamino)-5-(1H-benzo[d]imidazol-2-yl)pentylcarbamate (295)

Acetic acid (6 mL) was added to 294 (1.55 g, 3.3 mmol) and heated at 90° C. for 20 minutes. The solvent was evaporated under reduced pressure. The residue was then purified by silica gel column chromatography with gradient of EtOAc (20-100%) in hexane to afford 295 (1.49 g, 99%) as a light yellow oil. LRMS (ESI): (calc.) 452.2; (found) 453.2 (MH)⁺.

### Step 3: (S)-benzyl 5-amino-5-(1H-benzo[d]imidazol-2-yl)pentylcarbamate (296)

Following the same procedure as described for compound 2 (scheme 1, example 1, step 2) but substituting 295 for 1 to afford 296 (1.1 g, 95%) as a yellow oil. LRMS (ESI): (calc.) 352.2; (found) 353.1(MH)⁺.

### Step 4 (297) (S)-benzyl 5-(1H-benzo[d]imidazol-2-yl)-5-(4-fluorobenzamido)pentylcarbamate

Following the same procedure as described for compound 54 (scheme 2, example 47, step 1) but substituting 296 for 2 and 4-fluorobenzoyl chloride for 3-bromopropanoyl chloride to afford 297 (165 mg, 81%) as a deep yellow oil. LRMS (ESI): (calc.) 474.2; (found) 475 (MNa)⁺.

### Step 5 (298) (S)-N-(5-amino-1-(1H-benzo[d]imidazol-2-yl)pentyl)-4-fluorobenzamide

Following the same procedure as described for compound 74 (step 8, scheme 5, example 55) but substituting 297 for 73 to afford 298 (119 mg, 99%) as a yellow oil. LRMS (ESI): (calc.) 340.1; (found) 341.2 (MH)⁺.

### Step 6: (S)-tert-butyl 4-(2-(5-(1H-benzo[d]imidazol-2-yl)-5-(4-fluorobenzamido)pentylamino)-2-oxoethylthio)phenylcarbamate (299a)

Following the same procedure as described for compound 1 (step 1, scheme 1, example 1) but substituting acid 128 and amine 298 for N-Boc-caproic acid and 3-phenyl aniline, respectively, to afford 299a (175 mg, 99%) as a yellow oil. LRMS (ESI): (calc.) 605.2 (found) 606.7 (MH)⁺.

### EXAMPLE 111a

### (S)-N-(5-(2-(4-aminophenylthio)acetamido)-1-(1H-benzo[d]imidazol-2-yl)pentyl)-4-fluorobenzamide (300a)

### Step 7: (S)-N-(5-(2-(4-aminophenylthio)acetamido)-1-(1H-benzo[d]imidazol-2-yl)pentyl)-4-fluorobenzamide (300a)

Following the same procedure as described for compound 2 (scheme 1, example 1, step 2) but substituting 299a for 1 to afford 300a (15 mg, 9%) as a yellow oil. (MeOD-d4) δ (ppm):7.98 (qobs, J=6.0, 9.0 Hz, 2H); 7.55-7.53 (m, 2H); 7.24-7.15 (m, 6H); 6.61(d, J=9 Hz, 2H); 5.34 (qobs, J=6.0, 9.0 Hz, 1H); 3.31 (s, 2H); 3.15-3.11 (m, 2H); 2.18-2.02 (m, 2H); 1.52-1.27 (m, 4H) LRMS (ESI): (calc.) 505.1; (found) 506.5(MH)⁺.

### EXAMPLE 110b,c,d

Example 110b,c,d describe the preparation of compound 299b,c,d using the same procedures as described for compound 299a in Example 111a. Characterization data are presented in a Table 24.

TABLE 24

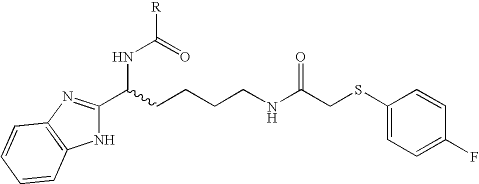

| Ex | Cpd | R | Stereo chemistry | Name | Characterization | Scheme (step) |
|---|---|---|---|---|---|---|
| 110b | 299b | 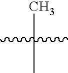 | S | (S)-N-(5-acetamido- 5-(1H-benzo-[d]imidazol- 2-yl)pentyl)-2-(4-fluoro- phenylthio)acetamide | (MeOD-d4) δ(ppm): 7.52 (bs, 2H); 7.39 (m, 2H); 7.20 (m, 2H); 7.02 (t, 2H, J=8.8 Hz); 5.09 (dd, | |
| # J=6.3, 8.6 Hz, 1H); 3.50 (s, 2H); 3.14 (t, J=6.8, 13.6 Hz, 2H); 2.10-2.01 (m, 4H); 1.93-1.83 (m, 1H); 1.50-1.43 (m, 2H); 1.37-1.24 (m, 2H) LRMS (ESI): 428.1 (calc) 429.8 (found) | 48 (1-6) | | | | | |
| 110c | 299c | 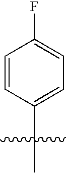 | R | (S)-N-(1-(1H- benzo[d]-imidazol- 2-yl)-5-(2-(4- fluorophenylthio)- acetamido)pentyl)-4- fluorobenzamide | (MeOD-d4) δ(ppm): 7.98 (q, J=5.5, 9.0 Hz, 2H); 7.53 (m, 2H); 7.38 | |
| # (q, J=5.3, 9.0 Hz, 2H); 7.21 (m, 4H); 7.01 (t, J=8.6 Hz, 2H); 5.35 (dd, J=6.0, 8.8 Hz, 1H); 3.48 (s, 2H); 3.15 (t, J=6.6 Hz, 2H); 2.11 (m, 2H); 1.42 (m, 4H) LRMS (ESI): 508.1 (calc) 509.8 (found) | 48 (1-6) | | | | | |
| 110d | 299d | 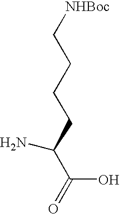 | 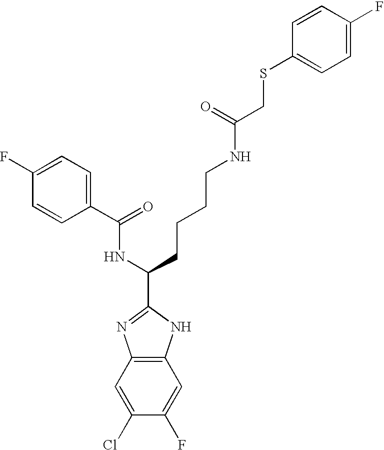 | (S)-N-(1-(5-chloro-6- fluoro-1H-benzo[d]- imidazol-2-yl)-5-(2-(4- fluorophenylthio)- acetamido)pentyl)-4- fluorobenzamide | MeOD-d4) δ(ppm): 7.28 (t, J=5.5 Hz, 2H); 7.58 (d, J=6.7 Hz, | |
| # 1H); 7.37 (m, 3H); 7.18 (t, J=9.8 Hz, 2H); 7.02 (t, J=8.8 Hz, 2H); 5.31 (t, J=9.6 Hz, 1H); 3.48 (s, 2H); 3.16 (t, J=6.7 Hz, 2H, J=6.7 Hz); 2.11 (m, 2H); 1.51 (m, 2H); 1.38 (m, 2H) LRMS (ESI): 560.3 (calc) 561.1 (MH)⁺. | 48 (1) 48(1-4) | | | | | |

### EXAMPLE 111e,f,g

Example 111e,f,g describe the preparation of compound 300e.f,g using the same procedures as described for compound 300a in Example 111a. Characterization data are presented in a Table 25.

TABLE 25

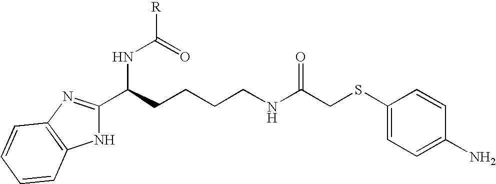

| | | | | | Scheme |
|---|---|---|---|---|---|
| Ex | Cpd | R | Name | Characterization | (step) |
| 111e | 300e | 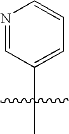 | (S)-N-(5-(2-(4- aminophenylthio) acetamido)-1-(1H- benzo[d]imidazol- 2-yl)pentyl)- nicotinamide | (MeOD-d4) d(ppm): 9.09 (s, 1H); 8.69 (d, J=5.0 Hz, 1H); 8.34 (d, J=8 Hz, 1H); 7.54 (m, 3H); 7.21 | |
| # (m, 2H); 7.16 (d, J=8 Hz, 2H); 6.61 (d, J=8 Hz, 2H); 5.35 (qobs, J=6, 9 Hz, 1H); 3.31 (s, 2H); 3.14 (m, 2H); 2.20-2.04 (m, 2H); 1.52-1.28 (m, 4H) LRMS (ESI): 488.2 (calc) 489.5 (found) | 48 | | | | |
| 111f | 300f | 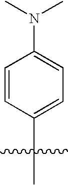 | (S)-N-(5-(2-(4- aminophenylthio) acetamido)-1-(1H- benzo[d]imidazol- 2-yl)pentyl)-4- (dimethylamino)- benzamide | (MeOD-d4) d(ppm): 7.81 (d, J=9Hz, 2H); 7.52 | |
| # (m, 2H); 7.19 (m, 4H); 6.74 (d, J=9 Hz, 2H); 6.61 (d, J=9 Hz, 2H); 5.35 (qobs, J=6, 8 Hz, 1H); 3.13 (m, 2H); 3.02 (s, 6H); 2.16-1.88 (m, 2H); 1.50-1.28 (m, 4H) LRMS (ESI): 530.2 (calc) 531.2 (found) | 48 | | | | |
| 111g | 300g | 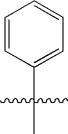 | (S)-N-(5-(2-(4- aminophenylthio) acetamido)-1-(1H- benzo[d]imidazol- 2-yl)pentyl)benzamide | (MeOD-d4) d(ppm): 7.91 (d, J=7 Hz, 2H); 7.56-7.52 (m, 2H); 7.49-7.45 (m, 2H); 7.22-7.19 (m, 2H); | |
| # 7.16 (d, J=9 Hz, 2H); 6.61 (d, J=9 Hz, 2H); 5.35 (qobs, J=6.0, 9.0 Hz, 1H); 3.34 (s, 2H); 3.15-3.11 (m, 2H), 2.18-2.06 (m, 2H), 1.48-1.29 (m, 4H) LRMS (ESI): 487.6 (calc) 488.5 (found) | 48 | | | | |

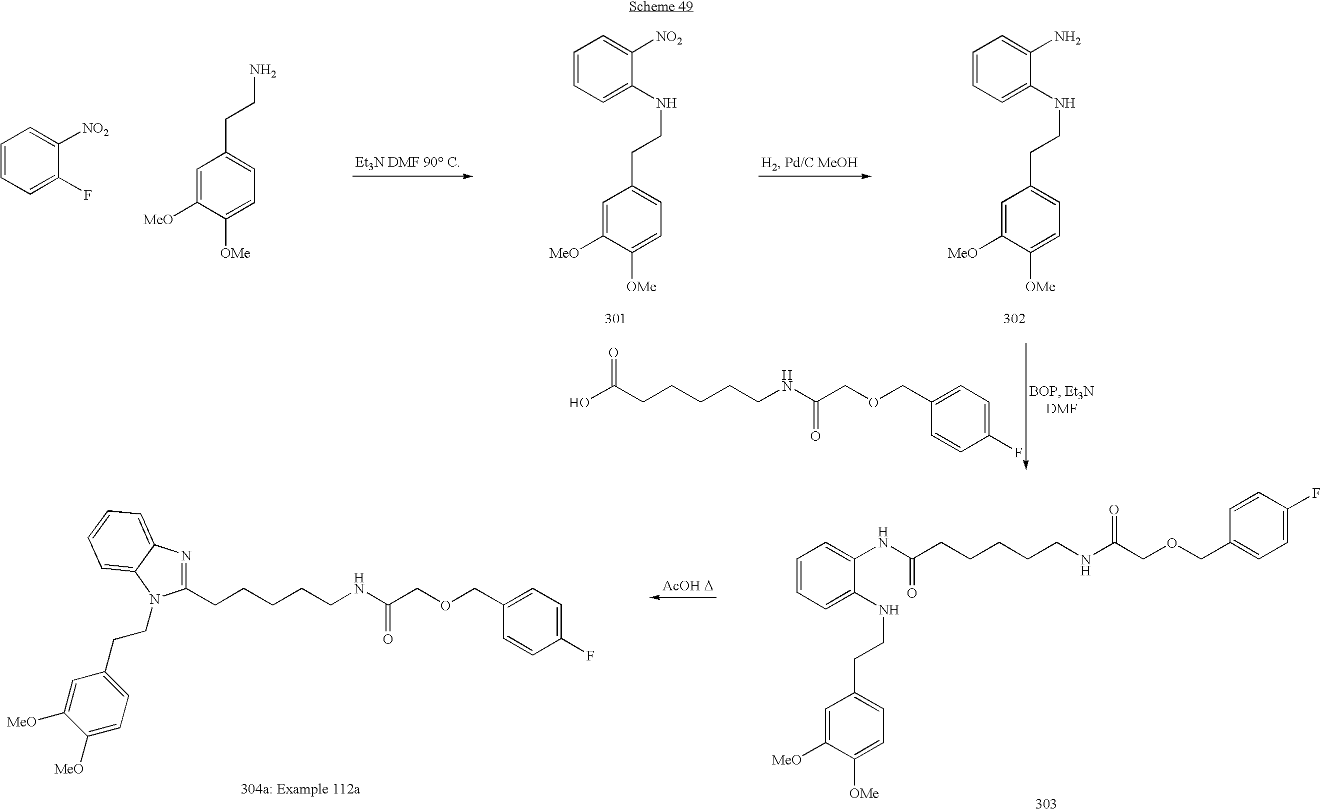

### EXAMPLE 112a

### N-(5-(1-(3,4-dimethoxyphenethyl)-1H-benzo[d]imidazol-2-yl)pentyl)-2-(4-fluorobenzyloxy)acetamide (304a)

### Step 1: N-(3,4-dimethoxyphenethyl)-2-nitroaniline (301)

2-(3,4-dimethoxyphenyl)ethanamine (0.83 mL, 4.96 mmol) was added to a solution of 1-fluoro-2-nitrobenzene (700 mg, 4.96 mmol) and Et₃N (2.0 mL, 15 mmol) in DMF (10 mL) and the reaction heated at 90° C. overnight. The reaction was then concentrated under reduced pressure and the resulting residue was partitioned between EtOAc and H₂O. The organic phase was separated, dried over Na₂SO₄, filtered and concentrated under reduced pressure. The residue was then purified by way of a silica plug to afford 301 (1.31 g, 87%) as bright yellow oil. LRMS (ESI): 302.1(calc) 325.1 (MNa)⁺.

### Step 2: N-1-(3,4-dimethoxyphenethyl)benzene-1,2-diamine (302)

Following the same procedure as described for compound 74 (scheme 5, example 55, step 1) but substituting 301 for 73 to afford 302 (725 mg, 62%) as a yellow oil. LRMS (ESI): (calc.) 272.1; (found) 273 (MH)⁺.

### Intermadiate: 6-(2-(4-fluorobenzyloxy)acetamido)hexanoic acid:

Following the same procedure as described for compound 61 (scheme 3, example 52, step 1 and 2-1)) but substituting 140 for 2-(benzyloxy)acetic acid to afford 6-(2-(4-fluorobenzyloxy)acetamido)hexanoic acid (1.42 g, 98%) as a yellow oil. LRMS (ESI): (calc.) 297.1; (found) 304.1(MLi)+.

### Step 3: N-(2-(3,4-dimethoxyphenethylamino)phenyl)-6-(2-(4-fluorobenzyloxy)acetamido)hexanamide (303)

Following the same procedure as described for compound 1 (scheme 1, example 1, step 1) but substituting amine 302 and 6-(2-(4-fluorobenzyloxy)acetamido)hexanoic acid for N-Boc-caproic acid and 3-phenyl aniline, respecectively, to afford 303 (64 mg, 32%) deep yellow oil. LRMS (ESI): (calc.) 551.3; (found) 552.3 (MH)⁺.

### Step 4 (AA) N-(5-(1-(3,4-dimethoxyphenethyl)-1H-benzo[d]imidazol-2-yl)pentyl)-2-(4-fluorobenzyloxy)acetamide (304a)

Following the same procedure as described for compound 295 (scheme 48, example 110a, step 2) but substituting 303 for 294 to afford 304a (5.9 mg, 7%) as a yellow oil. (MeOD-d4) δ(ppm):7.56 (d, J=7.0 Hz, 1H); 7.47 (d, J=7.2 Hz, 1H); 7.38 (q, J=5.7, 8.2 Hz, 2H); 7.24 (m, 2H); 7.06 (t,J=8.8 Hz, 2H); 6.75 (d, 1H, J=8.2 Hz); 6.46 (d, J=8.0 Hz, 1H); 6.20 (s, 1H); 4.53 (s, 2H); 4.42 (t, J=6.1 Hz, 2H); 3.90 (s, 2H); 3.73 (s, 3H); 3.51 (s, 3H); 3.19 (t, J=14.1 Hz, 2H); 3.04 (t, J=12.1 Hz, 2H); 2.32 (t, J=15 Hz, 2H); 1.56 (m, 2H); 1.45 (m, 2H);1.26 (m, 2H) LRMS (ESI): (calc.) 533.2; (found) 534.3 (MH)⁺.

### EXAMPLE 112b-e

Examples 112b-d describe the preparation of compounds 304b-d using the same procedures as described for compound 304a in Example 112a. Characterization data are presented in a Table 26.

TABLE 26

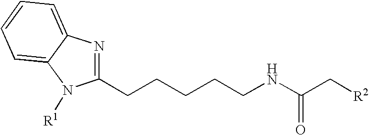

| | | | | | | Scheme |
|---|---|---|---|---|---|---|
| Ex | Cpd | R¹ | Name | Characterization | R² | (step) |
| 112b | 304b | 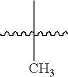 | 2-(4-fluoro- benzyloxy)-N- (5-(1-methyl-1H- benzo[d]imidazol- 2-yl)pentyl)acetamide | (MeOD-d4) d(ppm): 7.55 (d, J=7.6 Hz, 1H); 7.44 (d, J=8.9 Hz, 1H); 7.36 (m, 2H); 7.23 (m, 2H); 7.06 (t, J=8.8 Hz, 2H); | | |
| # 4.50 (s, 2H); 3.87 (s, 2H); 3.79 (s, 3H); 3.24 (t, J=6.8 Hz, 2H); 2.94 (t, J=7.6 Hz, 2H); 1.85 (m, 2H); 1.57 (m, 2H); 1.44 (m, 2H) LRMS: 383.2 (calc) 384.30 (found) | 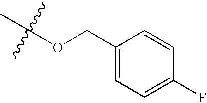 | 49 | | | | |
| 112c | 304c | 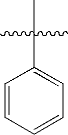 | 2-(4-fluorobenzyloxy)- N-(4-(1-phenyl-1H- benzo[d]imidazol-2- yl)butyl)acetamide | (MeOD-d4) d(ppm): 7.53 (m, 4H); 7.35 (d, J=7.0 Hz, 2H); 7.26 (q, J=5.5, 7.8 Hz, 2H); | | |
| # 7.15 (m, 2H); 6.97 (m, 3H); 4.41 (s, 2H); 3.77 (s, 2H); 3.05 (t, J=7.0 Hz, 2H); 2.72 (t, J=7.4 Hz, 2H); 1.6 (m, 2H); 1.34 (m, 2H); 1.18 (m, 4H) LRMS: 445.5 (calc) 446.2 (found) | 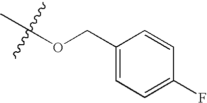 | 49 | | | | |
| 112d | 304d | 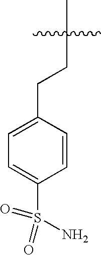 | 2-(4-fluorobenzyloxy)- N-(5-(1-(4-sulfamoyl- phenethyl)-1H- benzo[d]-imidazol-2- yl)pentyl)acetamide | (MeOD-d4) d(ppm): 7.72 (d, J=8.4 Hz, 2H); 7.56 (d, J=5.5 Hz, 1H); 7.49 (d, J=7.2 Hz, | | |
| # 1H); 4.53 (s, 2H); 4.49 (t, J=13.1 Hz, 2H); 3.89 (s, 2H), 3.20 (t, J=7.2 Hz, 4H); 2.40 (t, J=15.4 Hz, 2H); 1.60 (m, 2H): 1.48 (m, 2H); 1.29 (m, 2H) LRMS: 552.22 (calc) 553.26 (found) | 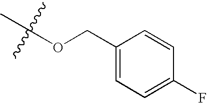 | 49 | | | | |
| 112e | 304e | H | N-(5-(1H-benzo[d]- imidazol-2-yl)pentyl)- 2-(4-fluorobenzyloxy)- acetamide | (MeOD-d4) d(ppm): 7.47 (m, 2H); 7.37 (m, 2H); 7.17 (m, 2H); 7.06 (t, J=8.9 Hz, 2H); 4.50 | | |
| # (s, 2H); 3.88 (s, 2H); 3.22 (t, J=7.0 Hz, 2H); 2.89 (t, J=7.4 Hz, 2H); 1.86 (m, 2H); 1.57 (m, 2H); 1.38 (m, 2H) LRMS: 369.1 (calc) 370.2 (found) | 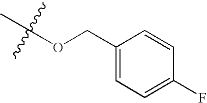 | 49 | | | | |

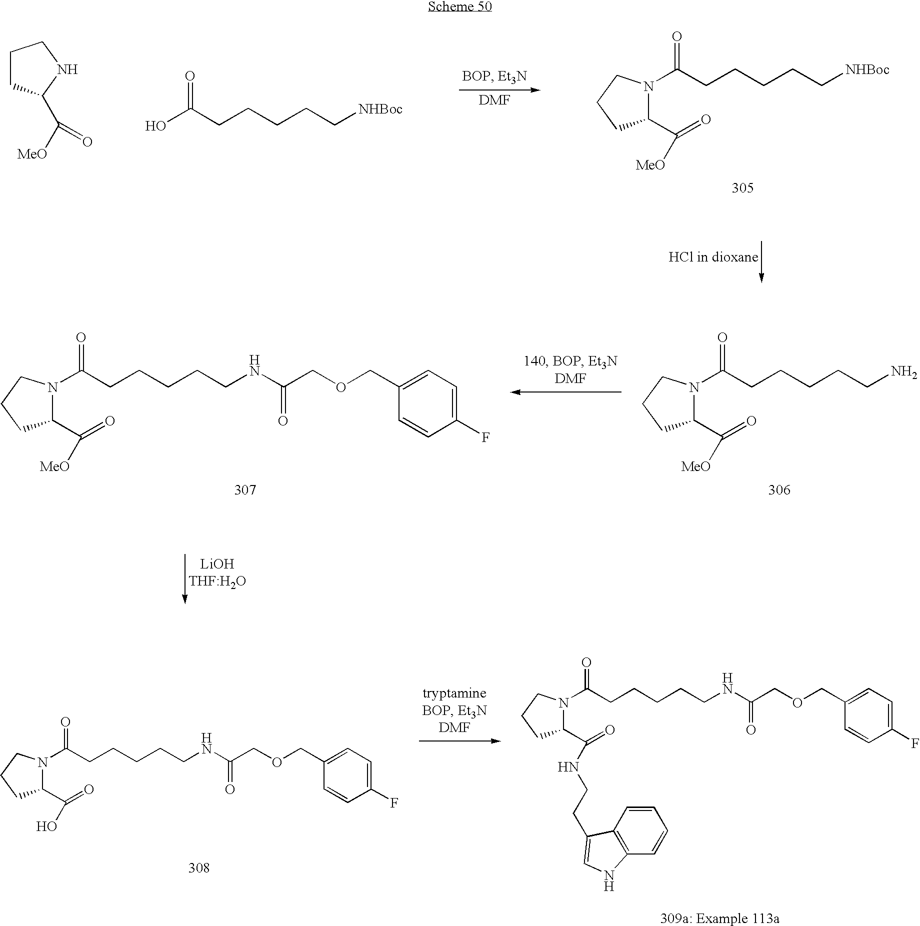

### EXAMPLE 113a

### (S)-N-(2-(1H-indol-3-yl)ethyl)-1-(6-(2-(4-fluorobenzyloxy)acetamido)hexanoyl)pyrrolidine-2-carboxamide (309a)

### Step 1: (S)-methyl 1-(6-(tert-butoxycarbonylamino)hexanoyl)pyrrolidine-2-carboxylate (305)

Following the same procedure as described for compound 1 (step 1, scheme 1, example 1) but substituting proline methyl ester for 3-phenyl aniline to afford 305 (1.7 g, 56% yield) as a pale yellow oil. LRMS (ESI): (calc.) 342.2; (found) 343.2(MH)⁺.

### Step 2: (S)-methyl 1-(6-aminohexanoyl)pyrrolidine-2-carboxylate (306)

Following the same procedure as described for compound 213 (scheme 31, example 92, step 3-1)) but substituting 305 for 212 to afford 306 (1 g, 71%) as a white solid. LRMS (ESI): (calc.) 242.1; (found) 243.0(MH)⁺.

### Step 3: (S)-methyl 1-(6-(2-(4-fluorobenzyloxy)acetamido)hexanoyl)pyrrolidine-2-carboxylate (307)

Following the same procedure as described for compound 1 (scheme 1, example 1, step 1) but substituting 140 and 306 for N-Boc-caproic acid and 3-phenyl aniline, respectively, to afford 307 (998 mg, 68%) as a pale yellow oil. LRMS (ESI): (calc.) 408.2; (found) 409.3 (MH)⁺.

### Step 4: (S)-1-(6-(2-(4-fluorobenzyloxy)acetamido)hexanoyl)pyrrolidine-2-carboxylic acid (308)

Following the same procedure as described for compound 5 (scheme 1, example 1, step 5) but substituting 307 for 4 to afford 308 (737 mg, 76%) as a yellow oil. LRMS (ESI): (calc.) 394.1; (found) 401(MLi)⁺.

### Step 5: (S)-N-(2-(1H-indol-3-yl)ethyl)-1-(6-(2-(4-fluorobenzyloxy)acetamido)hexanoyl)pyrrolidine-2-carboxamide (309a)

Following the same procedure as described for compound 1 (scheme 1, example 1, step 1) but substituting acid 308 and tryptamine for N-Boc-caproic acid and 3-phenyl aniline, respectively, to afford 309a (11 mg, 12%) as a yellow oil. (MeOD-d4) δ(ppm):7.55 (d, J=7.6 Hz, 1H); 7.35 (m, 3H); 7.03 (m, 5H); 4.54 (s;rotamer 4.47 ppm, 2H); 4.31(m, 1H); 3.91 (s,rotamer 3.85 ppm, 2H); 3.50 (m, 4H); 3.19 (m, 2H); 2.30-1.80 (m, 6H); 1.58-1.17 (m, 6H) LRMS (ESI): 536.2 (calc) 537.4 (found).

### EXAMPLES 113b-f

Examples 113b-f describe the preparation of compounds 309b-f using the same procedures as described for compound 309a in Example 113a Characterization data are presented in a Table 27.

TABLE 27

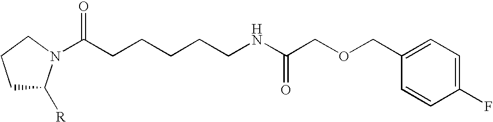

| | | | | | Scheme |
|---|---|---|---|---|---|
| Ex | Cpd | R | Name | Characterization | (step) |
| 113b | 309b | 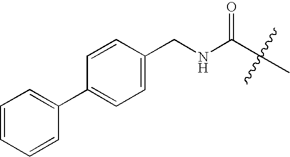 | (S)-N-(biphenyl-4- ylmethyl)-1-(6-(2- (4-fluorobenzyloxy)- acetamido)hexanoyl) pyrrolidine-2- carboxamide | (MeOD-d4) d(ppm): 7.57 (m, 4H); 7.37 (m, 7H); 7.06 (m, 2H); 4.54 (m, 2H); 4.42 (m, 3H); 3.90 s, 2H; | |
| # rotamer 3.86 ppm); 3.66-3.56 (m, 2H); 3.21-3.12 (m, 2H); 2.42-1.97 (m, 6H); 1.64-1.36 (m, 6H) LRMS: 559.2 (calc) 560.6 (found) | 50 | | | | |
| 113c | 309c | 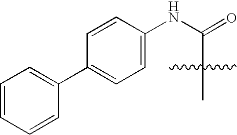 | (S)-N-(biphenyl-4- yl)-1-(6-(2-(4- fluorobenzyloxy)- acetamido)hexanoyl) pyrrolidine-2- carboxamide | (MeOD-d4) d(ppm): 7.62-7.56 (m, 6H); 7.37-7.28 (m, 5H); 7.06 (m, 2H); | |
| # 4.57-4.47 (m, 3H); 3.90 (s, 2H; rotamer 3.67 ppm); 3.67-3.58 (m, 2H); 3.22-3.14 (m, 2H); 2.40-2.02 (m, 6H); 1.62-1.29 (m, 6H) LRMS: 545.2 (calc) 546.6 (found) | 50 | | | | |
| 113d | 309d | 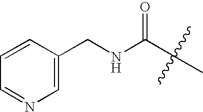 | (S)-1-(6-(2-(4- fluorobenzyloxy)- acetamido)hexanoyl)- N-(pyridin-3- ylmethyl)pyrrolidine- 2-carboxamide | (MeOD-d4) d(ppm): 8.45 (bm, 2H); 7.91-7.79 (m, 1H); 7.39 (t, J=12 Hz, 3H); 7.08 (t, J=8.8 Hz, | |
| # 2H); 4.56 (s, 2H); 4.41 (m, 3H); 3.92 (s, 2H); 3.58 (m, 2H); 3.21 (m, 2H); 2.38 (t, J=7.6 Hz, 2H); 2.19-1.93 (m, 4H); 1.63-1.33 (m, 6H) LRMS: 484.2 (calc) 485.3 (found) | 50 | | | | |
| 113e | 309e | 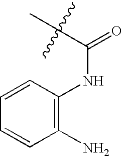 | (S)-N-(2-aminophenyl)- 1-(6-(2-(4-fluoro- benzyloxy)acetamido)- hexanoyl) pyrrolidine-2- carboxamide | (MeOD-d4) d(ppm): 7.41 (m, 2H), 7.10-7.01 (m, 4H); 6.79 (d, J=7.2 Hz, | |
| # 1H); 6.65 (d, J=7.2 Hz, 1H); 4.54 (s, 2H); 4.50 (m, 1H); 3.90 (s, 2H); 3.66 (m, 2H); 3.22 (m, 2H); 2.41-2.01 (m, 6H); 1.65-1.35 (m, 6H) LRMS: 484.2 (calc) 485.3 (found) | 50 | | | | |
| 113f | 309f | 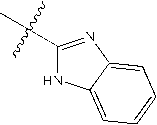 | (S)-N-(6-(2-(1H- benzo[d]imidazol- 2-yl)pyrrolidin-1-yl)- 6-oxohexyl)-2-(4- fluorobenzyloxy)- acetamide | (MeOD-d4) d(ppm): 7.49 (m, 2H); 7.38 (m, 2H); 7.18 (m, 2H); 7.07 (m, | |
| # 2H); 5.27 (d, J=8 Hz, 1H); 4.53 (s, 2H); 3.90 (s, 2H); 3.90 (s,2H; rotamer 3.87); 3.63 (m, 1H); 3.22 (t, J=4.4 Hz, 2H); 3.02 (m, 1H); 2.60-2.00 (m, 6H); 1.84-1.08 (m, 6H) LRMS: 466.2 (calc) 467.3 (found) | 50 48 (2) | | | | |

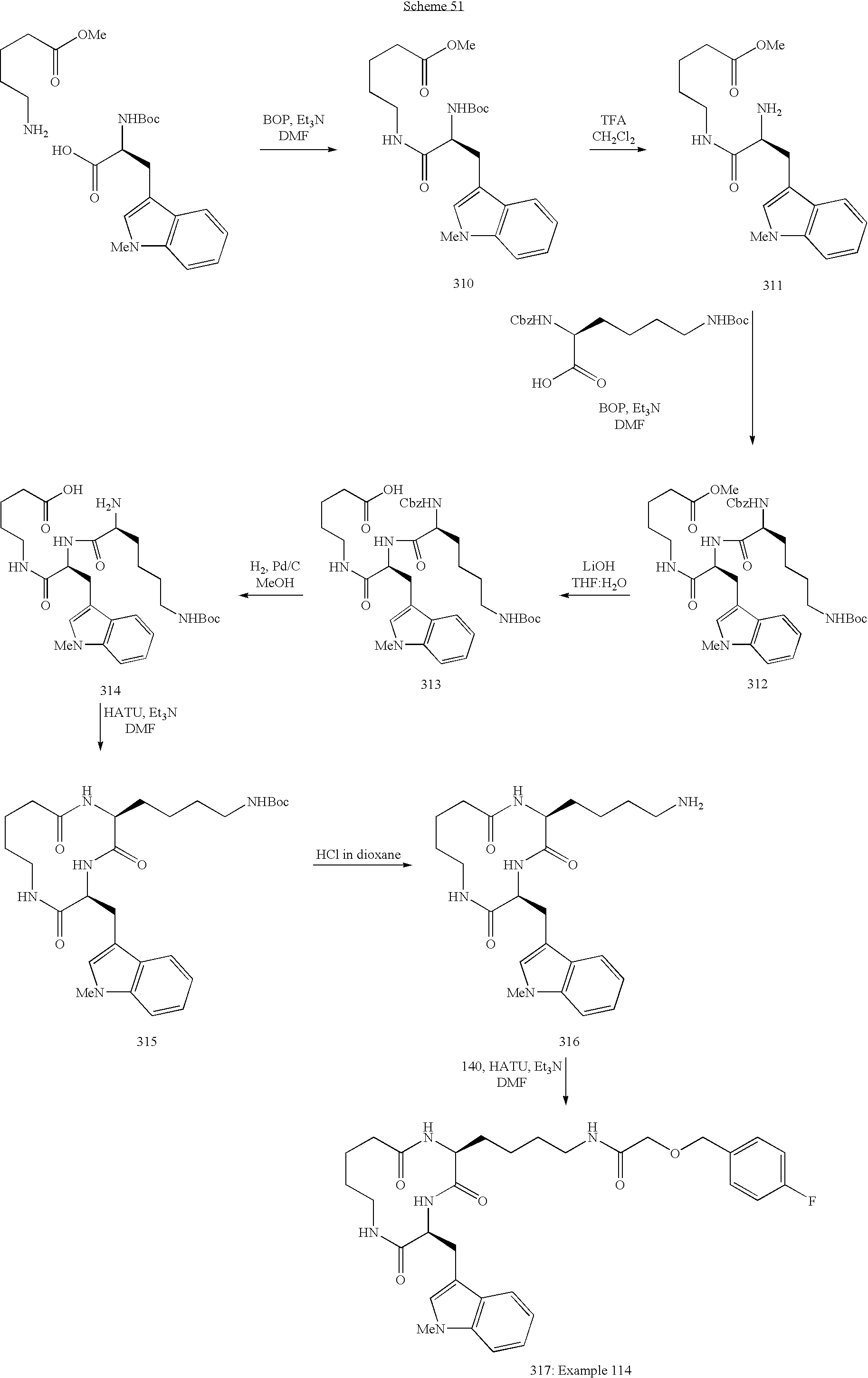

### EXAMPLE 114

### 2-(4-fluorobenzyloxy)-N-(4-((2S,5S)-5-((1-methyl-1H-indol-3-yl)methyl)-3,6,12-trioxo-1,4,7-triazacyclododecan-2-yl)butyl)acetamide (317)

### Step 1: (S)-methyl 5-(2-(tert-butoxycarbonylamino)-3-(1-methyl-1H-indol-3-yl)propanamido)pentanoate (310)

Following the same procedure as described for compound 1 (scheme 1, example 1, step 1) but substituting (S)-2-(tert-butoxycarbonylamino)-3-(1-methyl-1H-indol-3-yl)propanoic acid and methyl 5-aminopentanoate for N-Boc-caproic acid and 3-phenyl aniline, respectively, to afford 310 (1.42 g, 96%) as a yellow oil. LRMS (ESI): (calc.) 431.2; (found) 432.1 (MH)⁺.

### Step 2: (S)-methyl 5-(2-amino-3-(1-methyl-1H-indol-3-yl)propanamido)pentanoate (311)

Following the same procedure as described for compound 2 (scheme 1, example 1, step 2) but substituting 310 for 1 to afford amine 311 (1.05 g, 96%) as a white foam. LRMS (ESI): (calc.) 331.1; (found) 332.1 (MH)⁺.

### Step 3: (10S,13S)-methyl 10-(benzyloxycarbonylamino)-2,2-dimethyl-13-((1-methyl-1H-indol-3-yl)methyl)-4,11,14-trioxo-3-oxa-5,12,15-triazaicosan-20-oate (312)

Following the same procedure as described for compound 1 (scheme 1, example 1, step 1) but substituting (S)-2-(benzyloxycarbonylamino)-6-(tert-butoxycarbonylamino)hexanoic acid and amine 311 for N-Boc-caproic acid and 3-phenyl aniline, respectively, to afford 312 (2.16 g, 98%) as a yellow foam. LRMS (ESI): (calc.) 693.3; (found) 694.4(MH)⁺.

### Step 4: (10S,13S)-10-(benzyloxycarbonylamino)-2,2-dimethyl-13-((1-methyl-1H-indol-3-yl)methyl)-4,11,14-trioxo-3-oxa-5,12,15-triazaicosan-20-oic acid (313)

Following the same procedure as described for compound 5 (scheme 1, example 1, step 5) but substituting 312 for 4 to afford 313 (997 mg, 99%) as a clear oil. LRMS (ESI): (calc.) 679.3; (found) 680.4(MH)⁺.

### Step 5: (10S,13S)-10-amino-2,2-dimethyl-13-((1-methyl-1H-indol-3-yl)methyl)-4,11,14-trioxo-3-oxa-5,12,15-triazaicosan-20-oic acid (314)

Following the same procedure as described for compound 74 (scheme 5, example 55, step 3) but substituting 313 for 73 to afford 314 (759 mg, 96%) as a yellow oil. LRMS (ESI): (calc.) 545.3; (found) 546.6(MH)⁺.

### Step 6: tert-butyl 4-((2S,5S)-5-((1-methyl-1H-indol-3-yl)methyl)-3,6,12-trioxo-1,4,7-triazacyclododecan-2-yl)butylcarbamate (315)

A solution of HATU (43 mg, 0.11 mmol) in DMF (1 mL) was added to a solution of 314 (20 mg, 0.04 mmol) and Et₃N (53 μL) in DMF (3 mL).The reaction was stirred for 3 h, and then partitioned between EtOAc and H₂O. The organic phase was separated, dried (Na₂SO₄), filtered and concentrated under reduced pressure. The residue was purified by trituration with Et₂O to afford 315 (11 mg, 52%) as a white solid. LRMS (ESI): (calc.) 527.3; (found) 528.4(MH)⁺.

### Step 7: (3S,6S)-6-(4-aminobutyl)-3-((1-methyl-1H-indol-3-yl)methyl)-1,4,7-triazacyclododecane-2,5,8-trione (316)

Following the same procedure as described for compound 213 (scheme 31, example 92, step 3-1)) but substituting 315 for 212 to afford amine 316 (8 mg, 97%) as a white solid. LRMS (ESI): (calc.) 427.2; (found) 428.4(MH)⁺.

### Step 8: 2-(4-fluorobenzyloxy)-N-(4-((2S,5S)-5-((1-methyl-1H-indol-3-yl)methyl)-3,6,12-trioxo-1,4,7-triazacyclododecan-2-yl)butyl)acetamide (317)

Following the same procedure as described for compound 315 (scheme 51, example 114, step 6) but substituting 316 and 2-(4-fluorobenzyloxy)acetic acid for 314 to afford 317 (2 mg, 19%) as a white solid. (DMSO-d₆) δ (ppm): 8.00 (d, J=8.4 Hz, 1H); 7.84 (d, J=8. Hz, 1H); 7.76 (t, J=6.0 Hz, 1H); 7.50 (d, J=8.0 Hz, 1H); 7.39 (m, 2H); 7.32 (d, J=8.2 Hz, 1H); 7.17 (t, J=6.4 Hz, 2H); 7.08 (t, J=7.0 Hz, 1H); 6.98 (m, 2H); 6.89 (m, 1H); 4.47 (s, 2H); 4.27 (m, 1H); 3.86 (m, 3H); 3.68 (s, 3H); 3.20 (m, 2H); 2.96 (m, 3H); 2.28 (m, 1H); 1.86 (t, J=11.5 Hz, 1H); 1.70-1.02 (m, 11H) LRMS (ESI): (calc.) 593.3; (found) 428.4(MH)⁺. LRMS: 593.3 (calc) 594.6 (found)

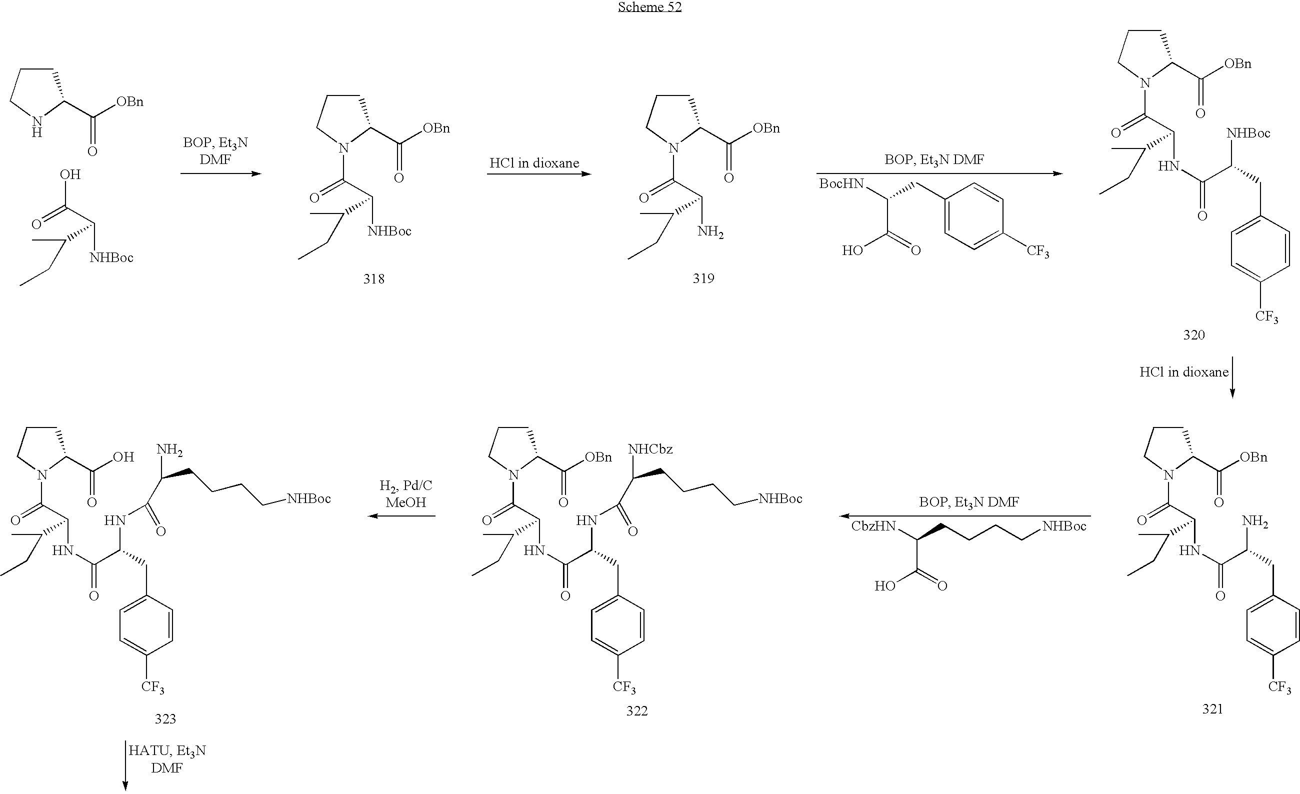

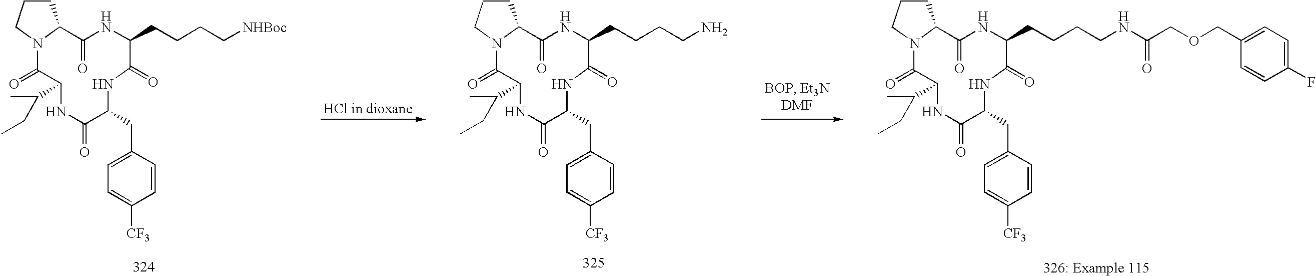

### EXAMPLE 115

### N-(4-((3S,6R,9S,14aR)-9-sec-butyl-1,4,7,10-tetraoxo-6-(4-(trifluoromethyl)benzyl)-tetradecahydropyrrolo[1,2-a][1,4,7,10]tetraazacyclododecin-3-yl)butyl)-2-(4-fluorobenzyloxy)acetamide (326)

### Step 1: (R)-benzyl 1-((2S,3R)-2-(tert-butoxycarbonylamino)-3-methylpentanoyl)pyrrolidine-2-carboxylate (318)

Following the same procedure as described for compound 1 (scheme 1, example 1, step 1) but substituting (2S,3R)-2-(tert-butoxycarbonylamino)-3-methylpentanoic acid and (R)-benzyl pyrrolidine-2-carboxylate for N-Boc-caproic acid and 3-phenyl aniline, respectively, to afford 318 (1.8 g, 82%) as a viscous clear oil. LRMS (ESI): (calc.) 418.2; (found) 419.3(MH)⁺.

### Step 2: (R)-benzyl 1-((2S,3R)-2-amino-3-methylpentanoyl)pyrrolidine-2-carboxylate (319)

Following the same procedure as described for compound 213 (scheme 31, example 92, step 3-1)) but substituting 318 for 212 to afford 319 (1.26 g, 98%) as a white foam. LRMS (ESI): (calc.) 318.1; (found) 319.2(MH)⁺.

### Step 3: (R)-benzyl 1-((2S,3R)-2-((R)-2-(tert-butoxycarbonylamino)-3-(4-(trifluoromethyl)phenyl)propanamido)-3-methylpentanoyl)pyrrolidine-2-carboxylate (320)

Following the same procedure as described for compound 1 (scheme 1, example 1, step 1) but substituting (R)-2-(tert-butoxycarbonylamino)-3-(4-(trifluoromethyl)phenyl)propanoic acid and 319 for N-Boc-caproic acid and 3-phenyl aniline, respectively, to afford 320 (1.5 g, 99%) as a viscous pale yellow oil. LRMS (ESI): (calc.) 633.3; (found) 634.2(MH)⁺.

### Step 4: (R)-benzyl 1-((2S,3R)-2-((R)-2-amino-3-(4-(trifluoromethyl)phenyl)propanamido)-3-methylpentanoyl)pyrrolidine-2-carboxylate (321)

Following the same procedure as described for compound 213 (scheme 31, example 92, step 3-1)) but substituting 320 for 212 to afford 321 (450 mg, 99%) as a white solid. LRMS (ESI): (calc.) 533.2; (found) 534.2(MH)⁺.

### Step 5: (R)-benzyl 1-((10R,13R,16S)-10-(benzyloxycarbonylamino)-16-sec-butyl-2,2-dimethyl-4,11,14-trioxo-13-(4-(trifluoromethyl)benzyl)-3-oxa-5,12,15-triazaheptadecane)pyrrolidine-2-carboxylate (322)

Following the same procedure as described for compound 1 (scheme 1, example 1, step 1) but substituting (S)-2-(benzyloxycarbonylamino)-6-(tert-butoxycarbonylamino)hexanoic acid and 321 for N-Boc-caproic acid and 3-phenyl aniline, respectively, to afford 322 (705 mg, 99%) as a viscous clear oil. LRMS (ESI): (calc.) 895.4; (found) 896.3(MH)⁺.

### Step 6: (R)-1-((10S,13R,16S)-10-amino-16-sec-butyl-2,2-dimethyl-4,11,14-trioxo-13-(4-(trifluoromethyl)benzyl)-3-oxa-5,12,15-triazaheptadecane)pyrrolidine-2-carboxylic acid (323)

Following the same procedure as described for compound 74 (scheme 5, example 55, step 3) but substituting 322 for 73 to afford 323 (67 mg, 36%) as a white solid. LRMS (ESI): (calc.) 671.3; (found) 672.2(MH)⁺.

### Step 7: tert-butyl 4-((3S,6R,9S,14aR)-9-sec-butyl-1,4,7,10-tetraoxo-6-(4-(trifluoromethyl)benzyl)-tetradecahydropyrrolo[1,2-a][1,4,7,10]tetraazacyclododecin-3-yl)butylcarbamate (324)

Following the same procedure as described for compound 315 (scheme 51, example 114, step 6) but substituting 323 for 314 to afford 324 (36 mg, 56%) as a white solid. LRMS (ESI): (calc.) 653.3; (found) 654.4(MH)⁺.

### Step 8: (3S,6R,9S,14aR)-3-(4-aminobutyl)-9-sec-butyl-6-(4-(trifluoromethyl)benzyl)-decahydropyrrolo[1,2-a][1,4,7,10]tetraazacyclododecine-1,4,7,10-tetraone (325)

Following the same procedure as described for compound 213 (scheme 31, example 92, step 3-1)) but substituting 324 for 212 to afford 214 (31 mg, 99%) as a white solid. LRMS (ESI): (calc.) 533.2; (found) 534.2(MH)⁺.

### Step 9: N-(4-((3S,6R,9S,14aR)-9-sec-butyl-1,4,7,10-tetraoxo-6-(4-(trifluoromethyl)benzyl)-tetradecahydropyrrolo[1,2-a][1,4,7,10]tetraazacyclododecin-3-yl)butyl)-2-(4-fluorobenzyloxy)acetamide (326)

Following the same procedure as described for compound 1 (scheme 1, example 1, step 1) but substituting 140 and amine 325 for N-Boc-caproic acid and 3-phenyl aniline, respectively, to afford 326 (15 mg, 36%) as a white solid. (MeOD-d₄) δ (ppm): 7.54 (d, J=7.8 Hz, 2H); 7.40 (m, 4H); 7.08 (t, J=8.8 Hz, 2H); 4.79 (d, J=7.2 Hz, 1H); 4.75 (t, J=7.8 Hz, 1H); 4.55(s, 2H); 4.46 (d, J=10.9 Hz, 1H); 4.29 (t, J=7.4 Hz, 1H); 3.98 (t, J=9.7 Hz, 1H); 3.91 (s, 2H); 3.19 (m, 3H); 2.94 (dd, J=8.2, 14.2 Hz, 1H); 2.32 (m, 1H); 2.19 (m, 1H); 2.01 (m, 1H); 1.90 (m, 1H); 1.81 (m, 1H); 1.68 (m, 1H); 1.64-1.42 (m, 4H); 1.32-1.06 (m, 3H); 0.85 (m, 6H). LRMS (ESI): (calc.) 719.3; (found) 720.3(MH)⁺.

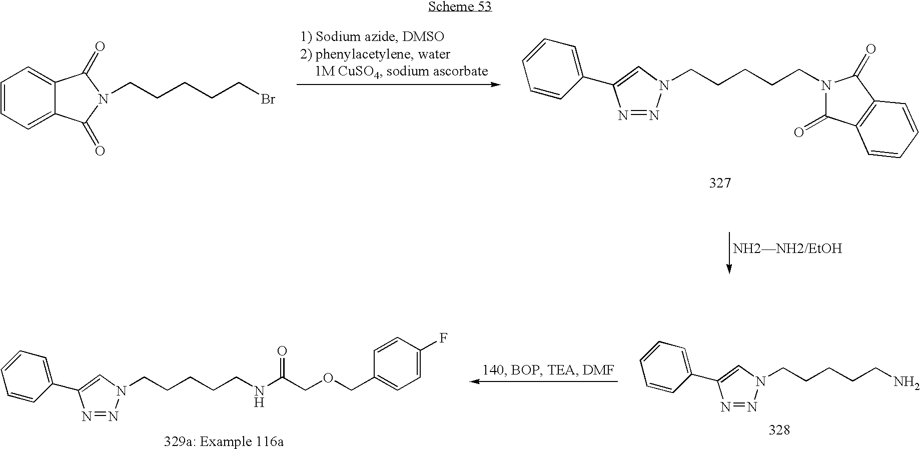

### EXAMPLE 116a

### 2-(4-fluorobenzyloxy)-N-(5-(4-phenyl-1H-1,2,3-triazol-1-yl)pentyl)acetamide (329a)

### Step 1: 2-(5-(4-phenyl-1H-1,2,3-triazol-1-yl)pentyl)isoindoline-1,3-dione (327)

N-(5-Bromopentyl)phthalimide (0.750 g, 2.54 mmol) was dissolved in a 0.5M solution of sodium azide in DMSO (5.0 mL, 2.5 mmol). After stirring 6 hours at room temperature, water (10.0 mL), sodium ascorbate (0.050 g, 0.25 mmol), phenylacetylene (0.255 g, 2.50 mmol) and a 1 M aqueous solution of copper sulfate (0.50 mL, 5.0 mmol) were added in that order. The reaction mixture was stirred at room temperature 16 hours and water (10.0 mL) was added until the product precipitated from the solution. The product was collected by filtration and then triturated in isopropyl ether to give 327 (0.530 g, 58%) as a green solid. LRMS (ESI): (calc) 381.2 (found) 382.3 (MH)⁺.

### Step 2: 5-(4-phenyl-1H-1,2,3-triazol-1-yl)pentan-1-amine (328)

Following the same procedure as described for compound 139 (step 5, scheme 16, example 73a) but substituting 327 for 138 to give 328 (0.350 g, 82%) as a beige solid. LRMS (ESI): (calc) 230.2 (found) 231.2 (MH)⁺.

### Step 3: 2-(4-fluorobenzyloxy)-N-(5-(4-phenyl-1H-1,2,3-triazol-1-yl)pentyl)acetamide (329a)

Following the same procedure as described for compound 1 (step 1, scheme 1, example 1) but substituting 2-(4-fluorobenzyloxy)acetic acid for N-Boc-caproic acid and 328 for 3-phenyl aniline to afford 329a (190 mg, 63%) as a white solid. (MeOD-d4) δ(ppm): 8.32 (s, 1H), 7.82-7.80 (m, 2H), 7.43 (t, J=7.4H, 2H), 7.41-7.33 (m, 3H), 7.06 (t, J=8.6 Hz, 2H), 4.51 (s, 2H), 4.46 (t, J=6.8 Hz, 2H), 3.89 (s, 2H), 3.24 (t, J=6.8 Hz, 2H), 2.03-1.95 (m, 2H), 1.62-1.55 (m, 2H), 1.39-1.31 (m, 2H). LRMS (ESI): (calc) 396.2 (found) 397.3 (MH)⁺.

### EXAMPLE 116b

Example 116b describe the preparation of compound 329b using the same procedures as described for compound 329a in Example 116a Characterization data are presented in a Table 28.

TABLE 28

| | | Starting | | | | Scheme |
|---|---|---|---|---|---|---|
| Ex | Cpd | Material | Product | Name | Characterization | (step) |
| 116b | 329b | 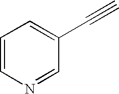 | 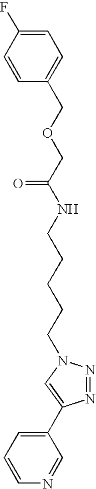 | 2-(4-fluorobenzyloxy)- N-(5-(4-(pyridin-3-yl)- 1H-1,2,3-triazol-1- yl)pentyl)acetamide | (MeOD-δ4) d(ppm): 9.00-9.01 (m, 1H), 8.49-8.52 (m, 2H), 8.26-8.28 (m, 1H), 7.50-7.53 (m, | |
| # 1H), 7.35-7.38 (m, 2H), 7.04-7.09 (m, 2H), 4.47-4.52 (m, 4H), 3.90 (s, 2H), 3.22-3.26 (m, 2H), 1.98-2.02 (m, 2H), 1.57-1.60 (m, 2H), 1.35-1.37 (m, 2H). LRMS (ESI): (calc) 397.2 (found) 398.2 | PG (1 (exB1) 16 (5 (Ex73a) 1 (1 (ex1) | | | | | |

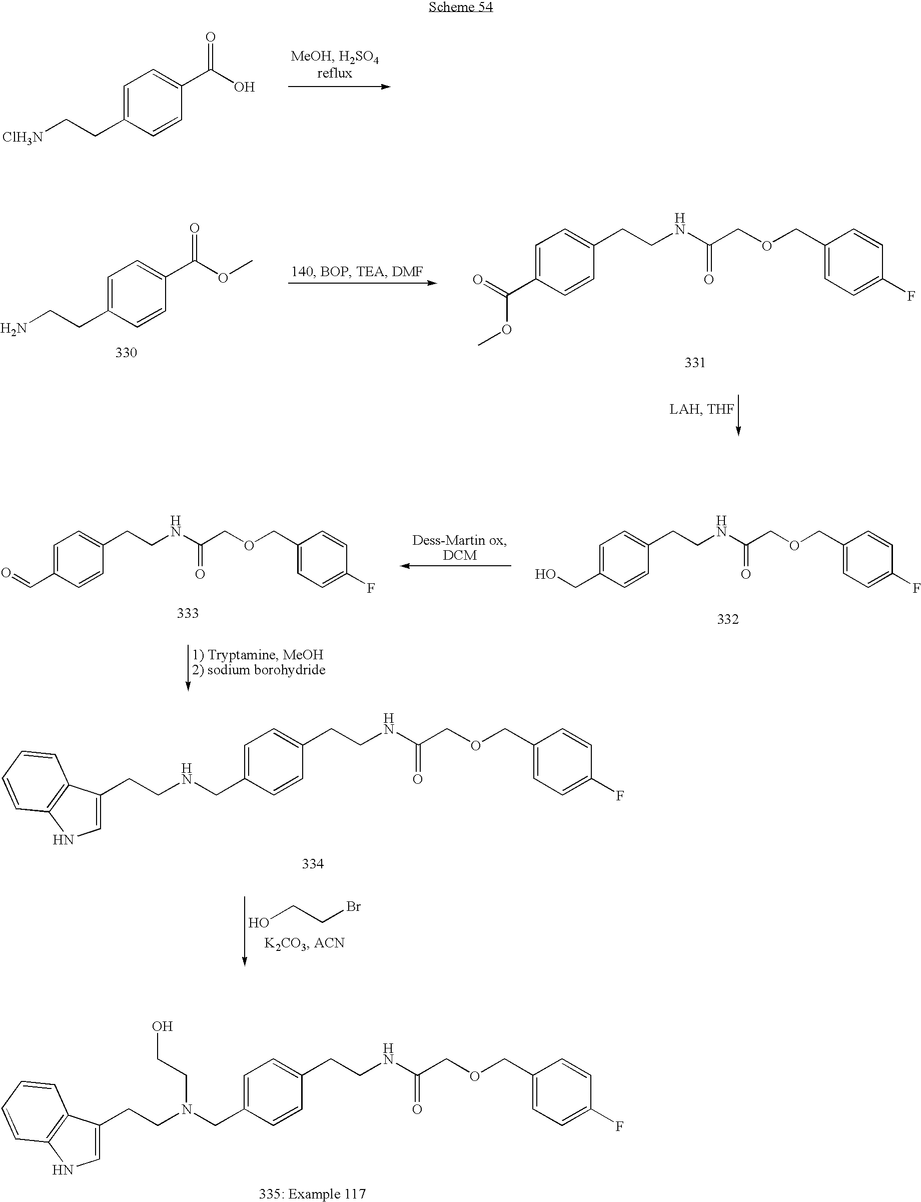

### EXAMPLE 117

### N-(4-(((2-(1H-indol-3-yl)ethyl)(2-hydroxyethyl)amino)methyl)phenethyl)-2-(4-fluorobenzyloxy)acetamide (335)

### Step 1: methyl 4-(2-aminoethyl)benzoate (330)

4-(2-aminoethyl)benzoic acid hydrochloride (1.00 g, 4.97 mmol) was dissolved in methanol (50.0 mL) and 6 drops of concentrated sulfuric acid ware added. After refluxing 16 h, the solution was concentrated under reduced pressure and the residue was dissolved in ethyl acetate. The organic phase was washed with sodium bicarbonate (ss), water and brine. The organic layer was dried over sodium sulfate, filtered and evaporated. The product was purified by silica gel column chromatography with 75% ethyl acetate in hexanes as to afford 330 (0.808 g, 91%) as a colorless oil. LRMS (ESI): (calc) 179.1 (found) 180.2 (MH)⁺.

### Step 2: methyl 4-(2-(2-(4-fluorobenzyloxy)acetamido)ethyl)benzoate (331)

Following the same procedure as described for compound 1 (step 1, scheme 1, example 1) but substituting 2-(4-fluorobenzyloxy)acetic acid for N-Boc-caproic acid and 330 for 3-phenyl aniline to afford 331 (0.753 g, 72%) as a yellow solid. LRMS (ESI): (calc) 345.1 (found) 346.2 (MH)⁺.

### Step 3: 2-(4-fluorobenzyloxy)-N-(4-(hydroxymethyl)phenethyl)acetamide (332)

Following the same procedure as described for compound 12 (step 5, scheme 1, example 7) but substituting 331 for 7c to afford 332 (0.880 g, 74%) as an off white solid. LRMS (ESI): (calc) 317.1 (found) 318.2 (MH)⁺.

### Step 4: 2-(4-fluorobenzyloxy)-N-(4-formylphenethyl)acetamide (333)

Following the same procedure as described for compound 195a (step 4, scheme 27, example 87a) but substituting alcohol 332 for alcohol 194a to afford 333 (0.672 g, 77%) as clear colorless oil. LRMS (ESI): (calc) 315.1 (found) 316.2 (MH)⁺.

### Step 5: N-(4-((2-(1H-indol-3-yl)ethylamino)methyl)phenethyl)-2-(4-fluorobenzyloxy)acetamide (334)

Following the same procedure as described for compound 103 (step 1, scheme 11, example 66) but substituting 333 for p-cyanobenzaldehyde to afford 334 (0.135 g, 14%) as a yellow solid. (MeOH-d4) δ(ppm): 7.49 (d, J=8.0 Hz, 1H), 7.33-7.29 (m, 3H), 7.19 (dd, J=12.9, 8.2 Hz, 4H), 7.08-6.98 (m, 5H), 4.45 (s, 2H), 3.87 (s, 2H), 3.80 (s, 2H), 3.45 (t, J=7.34 Hz, 2H), 3.01-2.91 (m, 4H), 2.80 (t, J=7.34 Hz, 2H). LRMS (ESI): (calc) 459.2 (found) 460.3 (MH)⁺.

### Step 6: N-(4-(((2-(1H-indol-3-yl)ethyl)(2-hydroxyethyl)amino)methyl)phenethyl)-2-(4-fluorobenzyloxy)acetamide (335)

Following the same procedure as described for compound 197b (step 1, scheme 27, example 88b) but substituting amine 334 for amine 196a to afford 335 (40 mg, 27%) as a yellow oil after purification by silica gel column chromatography with ethyl acetate. (MeOH-d4) δ(ppm): 7.37 (d, J=7.8 Hz, 1H), 7.30-7.27 (m, 5H), 7.17 (d, J=8.0 Hz, 2H), 7.07-7.02 (m, 3H), 6.97 (s, 1H), 6.95-6.91 (m, 1H), 4.43 (s, 2H), 3.87 (s, 2H), 3.73 (s, 2H), 3.64 (t, J=6.3 Hz, 2H), 3.48 (t, J=7.2 Hz, 2H), 2.95-2.91 (m, 2H), 2.83-2.73 (m, 6H). LRMS (ESI): (calc) 503.3 (found) 504.3 (MH)⁺.

### EXAMPLE 300-354

Example 300-354 describe the preparation of compound 500-554 using the same procedures as described for in the scheme column in the table below Characterization data are presented in a Table 29.

TABLE 29

| | | Starting | |
|---|---|---|---|
| Ex | Cpd | Material | Product |
| 300 | 500 | 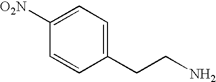 | 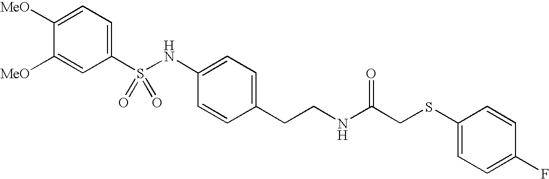 |
| 301 | 501 | 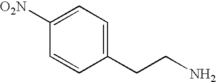 | 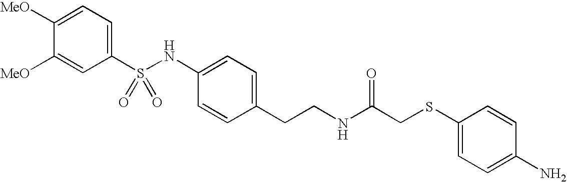 |
| 302 | 502 | 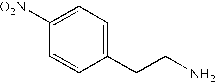 | 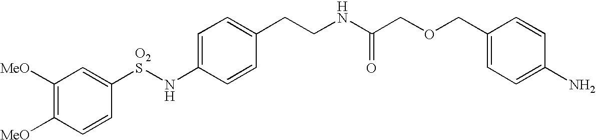 |
| 303 | 503 | 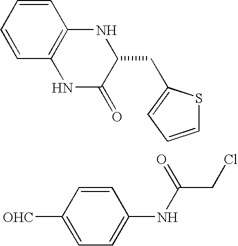 | 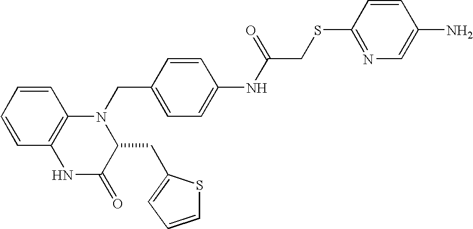 |
| 304 | 504 | 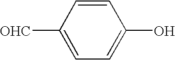 | 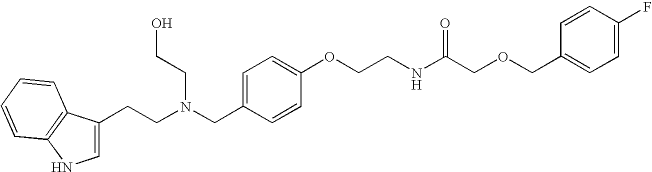 |
| 305 | 505 | 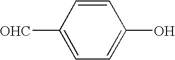 | 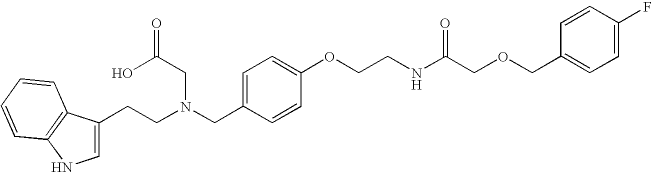 |
| 306 | 506 | 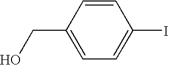 | 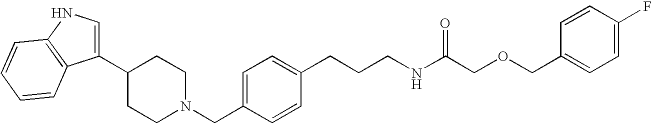 |
| 307 | 507 | 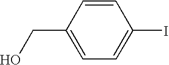 | 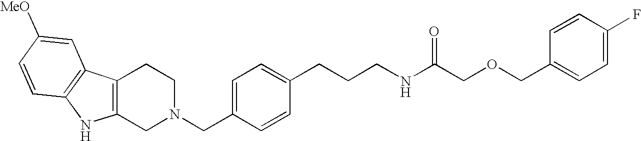 |
| 308 | 508 | 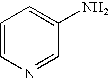 | 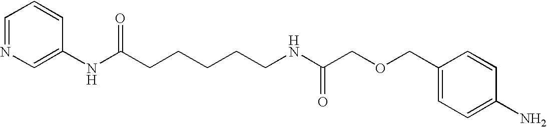 |
| 309 | 509 | 3b | 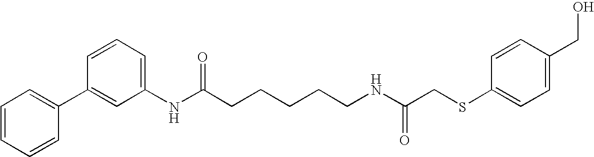 |
| 310 | 510 | 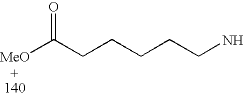 | 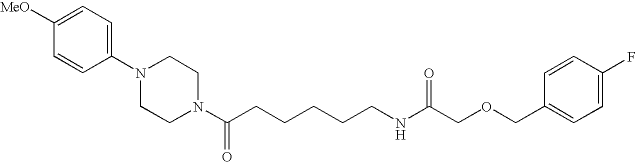 |
| 311 | 511 | 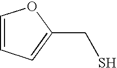 | 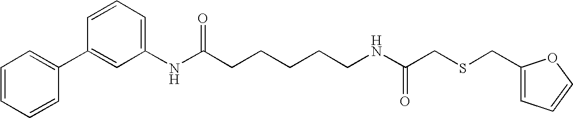 |
| 312 | 512 | 511 | 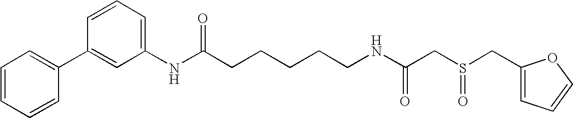 |
| 313 | 513 | 140 | 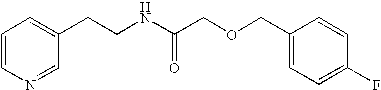 |
| 314 | 514 | 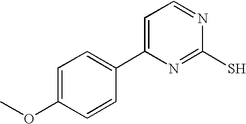 | 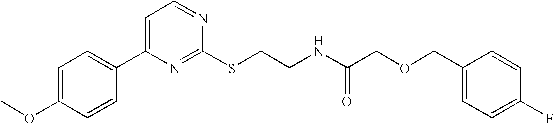 |
| 315 | 515 | 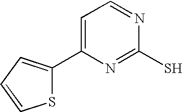 | 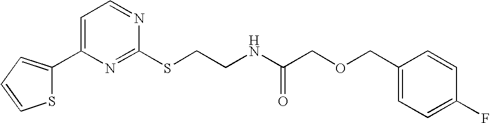 |
| 316 | 516 | 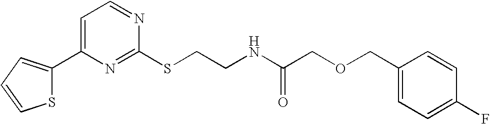 | 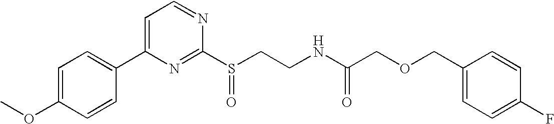 |
| 317 | 517 | 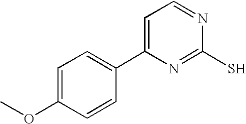 | 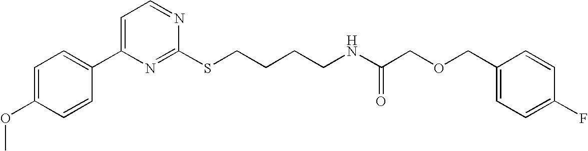 |
| 318 | 518 | 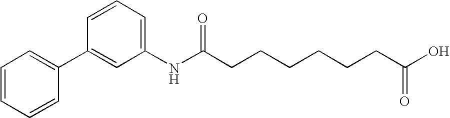 | 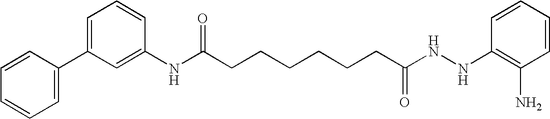 |
| 319 | 519 | 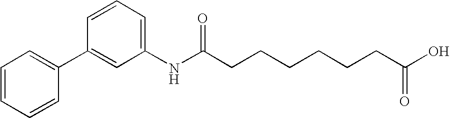 | 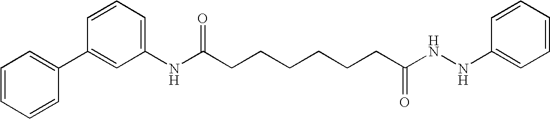 |
| 320 | 520 | 2 | 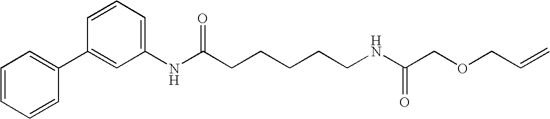 |
| 321 | 521 | 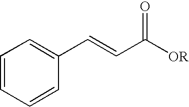 | 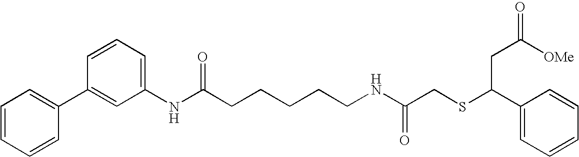 |
| 322 | 522 | 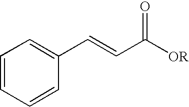 | 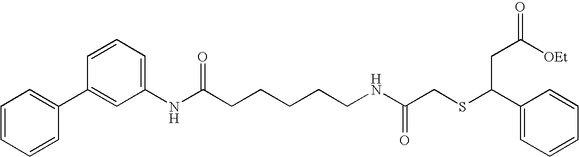 |
| 323 | 523 | 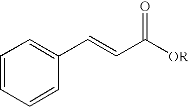 | 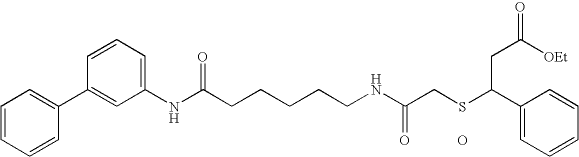 |
| 324 | 524 | 177 | 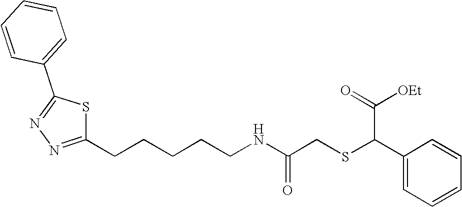 |
| 325 | 525 | 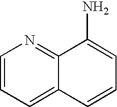 | 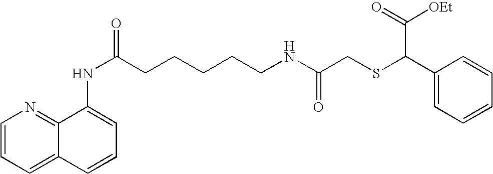 |
| 326 | 526 | 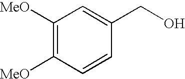 | 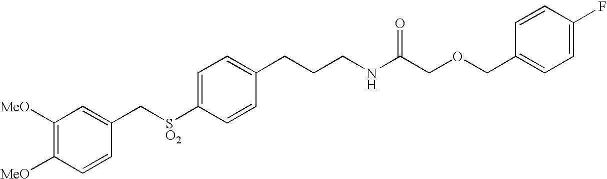 |
| 327 | 527 | 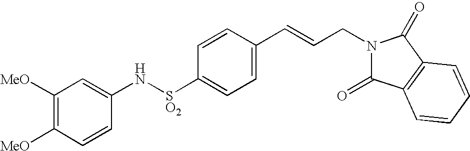 | 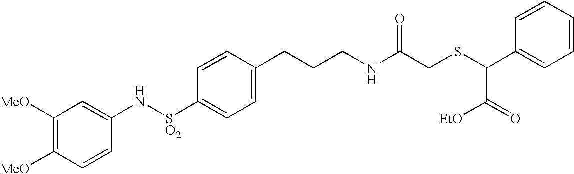 |
| 328 | 528 | 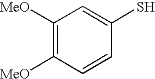 | 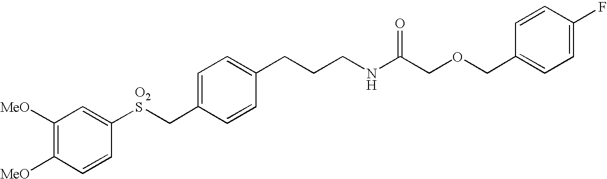 |
| 329 | 529 | 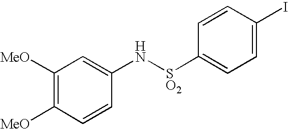 | 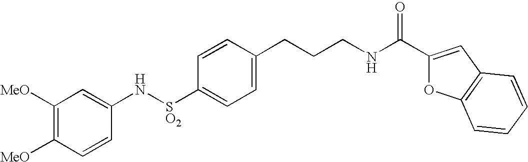 |
| 330 | 530 | 214 | 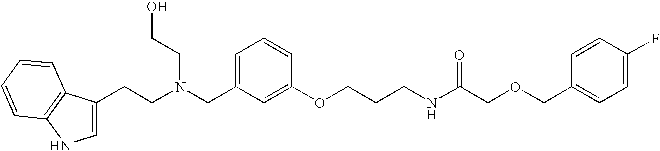 |
| 331 | 531 | 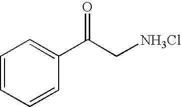 | 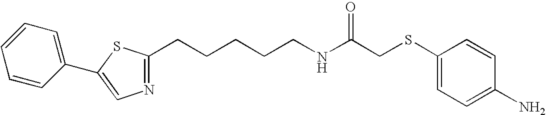 |
| 332 | 532 | 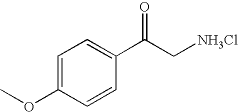 | 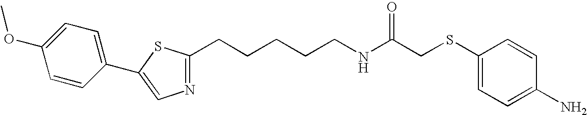 |
| 333 | 533 | 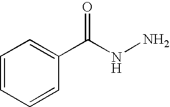 | 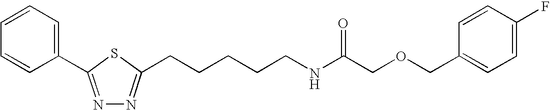 |
| 334 | 534 | 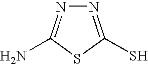 | 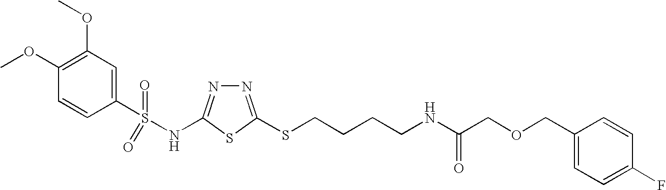 |
| 335 | 535 | 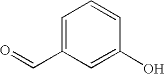 | 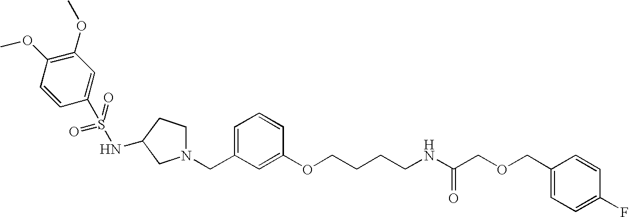 |
| 336 | 536 | 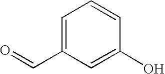 | 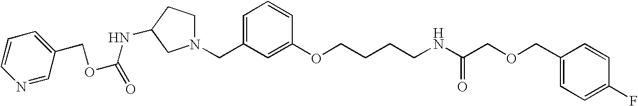 |
| 337 | 537 | 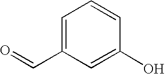 | 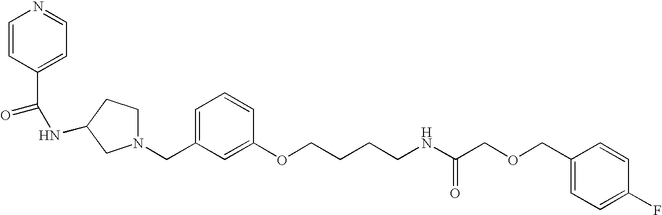 |
| 338 | 538 | 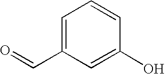 | 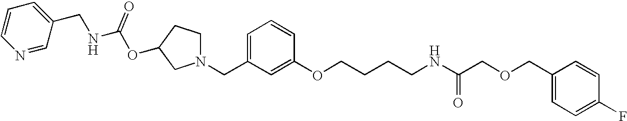 |
| 339 | 539 | 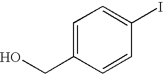 | 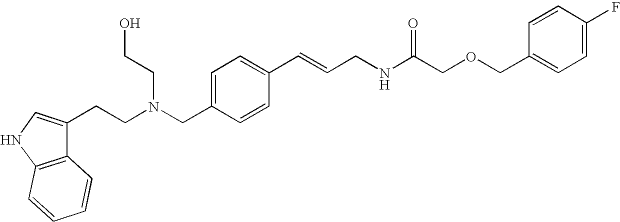 |
| 340 | 540 | 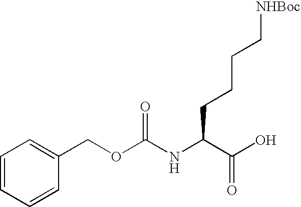 | 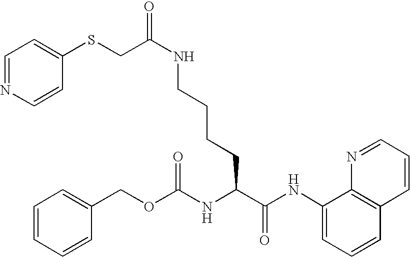 |
| 341 | 541 | 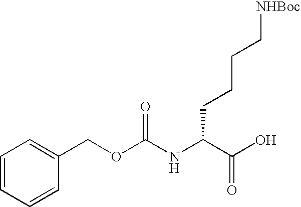 | 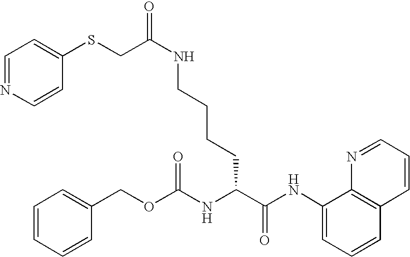 |
| 342 | 542 | 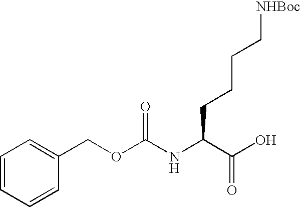 | 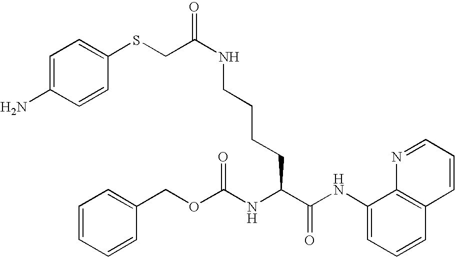 |
| 343 | 543 | 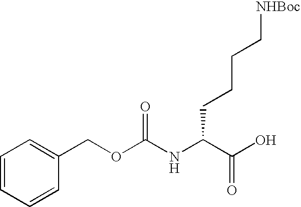 | 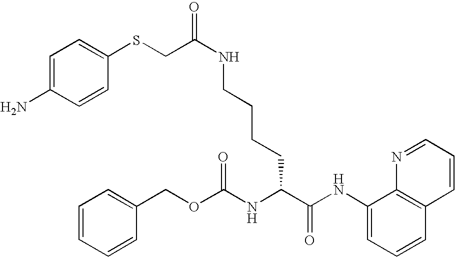 |
| 344 | 544 | 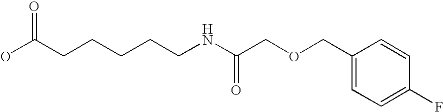 | 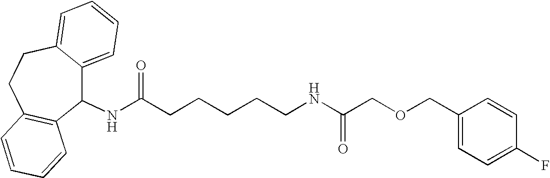 |
| 345 | 545 | 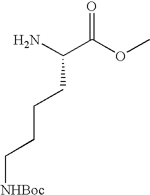 | 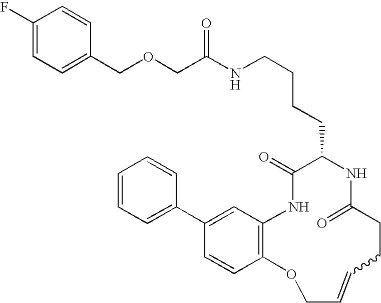 |
| 346 | 546 | 89284 | 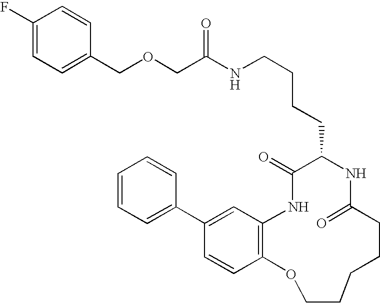 |
| 347 | 547 | 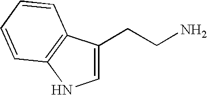 | 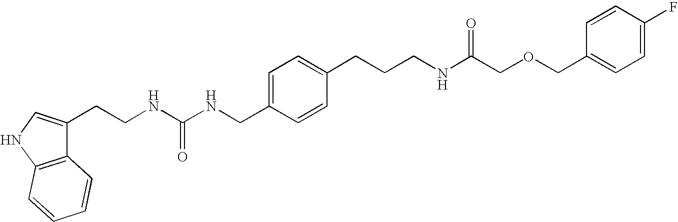 |
| 348 | 548 | 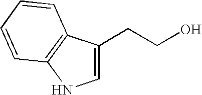 | 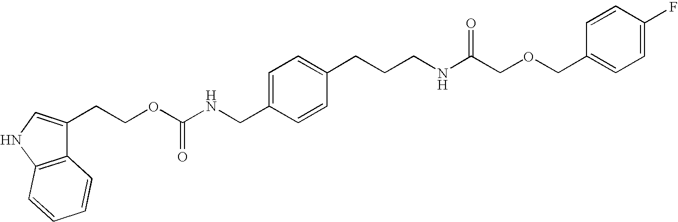 |
| 349 | 549 | 189 | 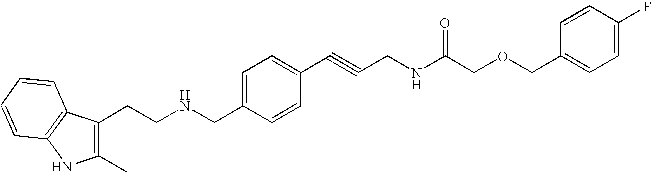 |
| 350 | 550 | 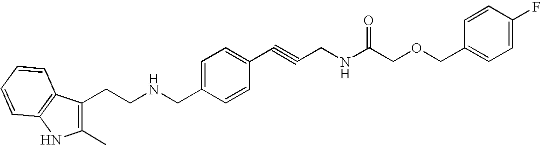 | 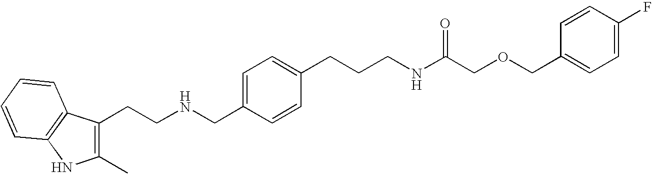 |
| 351 | 551 | 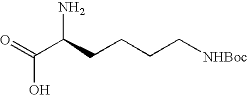 | 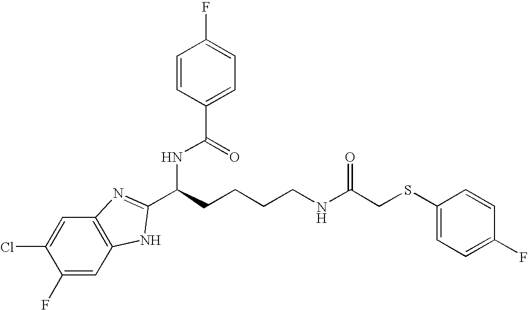 |
| 352 | 552 | 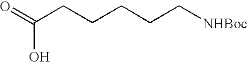 | 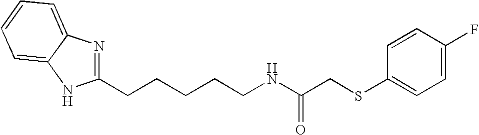 |
| 353 | 553 | 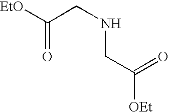 | 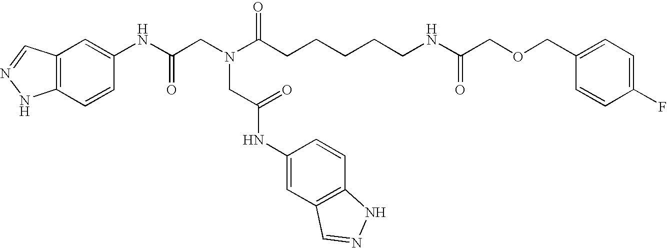 |
| 354 | 554 | 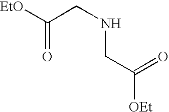 | 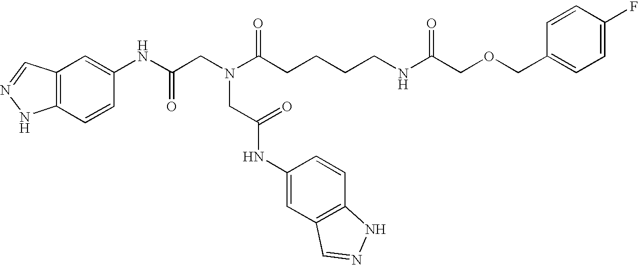 |

| | | | | Scheme |
|---|---|---|---|---|
| Ex | Cpd | Name | Characterization | (step) |
| 300 | 500 | N-(4-(3,4-dimethoxyphenyl | (MeOD-d4) δ (ppm) ¹H: 7.32-7.26 (m, 3H), 7.18 (d, J=2.2 Hz, | 1 (3) |
| | | sulfonamido)phenethyl)-2-(4- | 1H), 7.01-6.94 (m, 6H), 6.88 (d, J=8.6 Hz, 1H), 3.77 (s, 3H), 3.72 (s, | 2 (1) |
| | | fluorophenylthio)acetamide | 3H), 3.47 (s, 2H), 3.30 (t, J=7.2 Hz, 2H), 2.61 (1, J=7.2 Hz, 2H) | 18 (4) |
| | | | LRMS (ESI): (calc) 504.6; (found) 505.0 (MH)⁺. | 2 (1 (ex 47)) |
| 301 | 501 | 2-(4-aminophenylthio)-N-(4- | (MeOD-d4) δ (ppm) 1H: 10.02 (s, 1H), 7.98 (1, J=5.7 Hz, 1H), | 1 (1) |
| | | (3,4-dimethoxyphenylsulfonamido)- | 7.33 (dd, J=8.6, 2.2 Hz, 1H), 7.26 (d, J=2.3 Hz, 1H), | 15 (3) |
| | | phenethyl)acetamide | 7.16-7.02 (m, 7H), 6.58-6.49 (m, 2H), 5.29 (br s, 2H), | 2 (1 (ex 47)) |
| | | | 3.81 (s, 3H), 3.77 (s, 3H), 3.33 (s, 2H), 3.24-3.16 (m, 2H), | 1 (5 (ex 4)) |
| | | | 2.61-2.53 (m, 2H) | |
| | | | LRMS (ESI): (calc) 501.6; (found) 502.7 (MH)⁺. | |
| 302 | 502 | 2-(4-aminobenzyloxy)-N-(4-(3,4- | (MeOD-d4) δ (ppm): 7.30 (dd, J=8.4, 2.1 Hz, 1H), | 28 (1) |
| | | dimethoxyphenylsulfonamido)- | 7.18 (d, J=2.2 Hz, 1H), 7.08-7.00 (m, 6H), 6.92 | 5 (3) (ex 55) |
| | | phenethyl)acetamide | (d, J=8.6 Hz, 1H), 6.68 (d, J=8.4 Hz, 2H), 4.33 | 2 (1 (ex 47)) |
| | | | (s, 2H), 3.80 (s, 3H), 3.72 (s, 3H), 3.37 (t, J=7.2 | 1 (2) 1 (1) |
| | | | Hz, 2H), 2.70 (t, J=7.2 Hz, 2H). | 5 (3) (ex 55) |
| 303 | 503 | (R)-2-(5-aminopyridin-2-ylthio)-N- | (MeOD-d4) δ (ppm) ¹H: 7.97 (dd, J=2.9, 0.6 Hz, 1H), | 5 (1-3, 6) |
| | | (4-((3-oxo-2-(thiophen-2-ylmethyl)- | 7.45-7.40 (m, 2H), 7.24-7.07 (m, 4H), 7.02-6.98 (m, 1H), | 15 (3) |
| | | 3,4-dihydroquinoxalin-1(2H)- | 6.95-6.86 (m, 2H), 6.82-6.70 (m, 4H), 5.49 (s, 1H), 4.54 (d, | 1 (3, 4 (ex 1)) |
| | | yl)methyl)phenyl)acetamide | J=15.1 Hz, 1H), 4.12-4.02 (m, 2H), 4.00 (d, J=14.9 Hz, 1H), | |
| | | | 3.77 (s, 2H), 3.09-2.99 (m, 2H) | |
| | | | LRMS (ESI): (calc) 515.7; (found) 516.7 (MH)⁺. | |
| 304 | 504 | N-(2-(4-(((2-(1H-indol-3- | (MeOD-d4) δ (ppm) ¹H: 7.42-7.26 (m, 6H), 7.10-6.86 | 22 (1) |
| | | yl)ethyl)(2-hydroxyethyl)- | (m, 7H), 4.55 (s, 2H), 4.07 (t, J=5.7 Hz, 2H), 3.99 (s, 2H), | 27 (5) |
| | | amino)methyl) phenoxy)ethyl)-2- | 3.72 (s, 2H), 3.69-3.62 (m, 4H), 2.99-2.93 (m, 2H), 2.87-2.81 | 22 (1) |
| | | (4-fluorobenzyloxy)acetamide | (m, 2H), 2.77 (t, J=6.3 Hz, 2H) | 1 (2) 1 (1) |
| | | | LRMS (ESI): (calc) 519.6; (found) 520.6 (MH)⁺. | 27 (5) |
| 305 | 505 | 2-((2-(1H-indol-3-yl)ethyl)(4- | (MeOD-d4) δ (ppm) ¹H: 7.44-7.33 (m, 4H), 7.28 (d, | 22 (1) |
| | | (2-(2-(4-fluorobenzyloxy)acetamido) | J=8.6 Hz, 2H), 7.18-7.03 (m, 4H), 6.94 (t, J=7.8 Hz, 1H), | 27 (5) |
| | | ethoxy)benzyl)amino)acetic acid | 6.82 (d, J=8.6 Hz, 2H), 4.58 (s, 2H), 4.31 (s, 2H), 4.02 (t, | 22 (1) |
| | | | J=5.7 Hz, 2H), 4.01 (s, 2H), 3.76 (s, 2H), 3.66 (t, J=5.5 | 1 (2) 1 (1) |
| | | | Hz, 2H), 3.45 (t, J=7.6 Hz, 2H), 3.20 (t, J=7.2 Hz, 2H) | 1 (5 (ex 1)) |
| | | | LRMS (ESI): (calc) 533.6; (found) 534.6 (MH)⁺. | |
| 306 | 506 | N-(3-(4-((4-(1H-indol-3-yl)- | (MeOD-d4) δ (ppm) ¹H: 7.58 (d, J=8.0 Hz, 1H), | 27 (1 (ex 196b)) |
| | | piperidin-1-yl)methyl)phenyl)- | 7.41-7.31 (m, 3H), 7.29-7.24 (m, 2H), 7.20-7.15 (m, 2H), | |
| | | propyl)-2-(4-fluorobenzyloxy)- | 7.12-7.04 (m, 3H), 7.02-6.97 (m, 2H), 4.53 (s, 2H), 3.93 | |
| | | acetamide | (s, 2H), 3.52 (s, 2H), 3.26 (t, J=7.2 Hz, 2H), 3.03-2.94 | |
| | | | (m, 2H), 2.86-2.75 (m, 1H), 2.62 (t, J=8.0 Hz, 2H), | |
| | | | 2.22-2.10 (m, 2H), 2.04-1.94 (m, 2H), 1.90-1.76 (m, 4H) | |
| | | | LRMS (ESI): (calc) 513.6; (found) 514.7 (MH)⁺. | |
| 307 | 507 | 2-(4-fluorobenzyloxy)-N-(3- | (MeOD-d4) δ (ppm) ¹H: 7.44-7.37 (m, 2H), 7.34-7.30 | 27 (1 (ex 196b)) |
| | | (4-((6-methoxy-3,4-dihydro- | (m, 2H), 7.23-7.06 (m, 5H), 6.91 (d, J=2.5 Hz, 1H), 6.71 (dd, | |
| | | 1H-pyrido[3,4-b]indol-2(9H)- | J=8.8, 2.3 Hz, 1H), 4.57 (s, 2H), 3.94 (s, 2H), 3.81 (s, 3H), 3.73 (s, 2H), | |
| | | yl)methyl)phenyl)propyl)acetamide | 3.63 (s, 2H), 3.3.28 (t, J=7.2 Hz, 2H), 2.90-2.83 (m, 2H), 2.82-2.74 | |
| | | | (m, 2H), 2.65 (t, J=7.8 Hz, 2H), 1.90-1.80 (m, 2H) | |
| | | | LRMS (ESI): (calc) 515.6; (found) 516.4 (MH)⁺. | |
| 308 | 508 | 6-(2-(4-aminobenzyloxy)- | (MeOD-d4) δ (ppm): 8.71 (d, J=6.6 Hz, 1H), 8.22 (dd, J=4.9, | 1 (1) 1 (2) |
| | | acetamido)-N-(pyridin-3- | 1.6 Hz, 1H), 8.11-8.08 (m, 1H), 7.39-7.35 (m, 1H), 7.09 (d, J=8.6 Hz, | 1 (1) 5 (3) |
| | | yl)hexanamide | 2H), 6.69 (d, J=8.6 Hz, 2H), 4.43 (s, 2H), 3.85 (s, 2H), 3.23 (t, J=7.0 Hz, | (ex 55) |
| | | | 2H), 2.42 (t, J=7.4 Hz, 2H), 1.74 (quintet, J=7.4 Hz, 2H), 1.56 (quintet, | |
| | | | J=7.2 Hz, 2H), 1.44-1.38 (m, 2H). | |
| | | | LRMS (ESI): (calc) 370.2; (found) 371.2 (MH)⁺. | |
| 309 | 509 | N-(biphenyl-3-yl)-6-(2-(4- | (MeOD-d4) δ (ppm): 7.84 (s, 1H), 7.60-7.58 (m, 2H), 7.53-7.50 | 1 (4 (Ex 1)) |
| | | (hydroxymethyl)phenylthio)- | (m, 1H), 7.44-7.27 (m, 9H), 4.56 (s, 2H), 3.58 (s, 2H), 3.18 (t, J=6.8 | |
| | | acetamido)hexanamide | Hz, 2H), 2.35 (t, J=7.4 Hz, 2H), 1.69-1.63 (m, 2H), 1.49-1.43 (m, 2H), | |
| | | | 1.33-1.27 (m, 2H). | |
| | | | LRMS (ESI): (calc) 462.2; (found) 485.1 (MNa)⁺. | |
| 310 | 510 | 2-(4-fluorobenzyloxy)-N-(6- | (MeOD-d4) δ (ppm): 7.42-7.37 (m, 2H), 7.08 (t, J=8.8 Hz, 2H), | 1 (1) |
| | | (4-(4-methoxyphenyl)piperazin-1- | 6.94 (d, J=9.0 Hz, 2H), 6.83 (d, J=9.2 Hz, 2H), 4.55 (s, 2H), 3.92 | 1 (5 (Ex 1)) |
| | | yl)-6-oxohexyl)acetamide | (s, 2H), 3.73 (s, 3H), 3.68 (dt, J=15.6, 5.1 Hz, 4H), 3.24 (t, J=7.0 Hz, | 1 (1) |
| | | | 2H), 3.00 (dt, J=21.3, 5.1 Hz, 4H), 2.42 (t, J=7.6 Hz, 2H), 1.53 | |
| | | | (quintet, J=7.4 Hz, 2H), 1.55 (quintet, J=7.2 Hz, 2H), 1.40-1.32 (m, 2H). | |
| | | | LRMS (ESI): (calc) 471.3; (found) 472.5 (MH)⁺. | |
| 311 | 511 | N-(biphenyl-3-yl)-6-(2-(furan- | (MeOD-d4) δ (ppm): 7.38 (dd, J=8.6, 5.3 Hz, 2H), 7.19 (t, J=7.8 Hz, | 2 (2 (ex 47)) |
| | | 2-ylmethylthio)acetamido)- | 1H), 7.05 (t, J=8.8 Hz, 2H), 6.90-6.87 (m, 2H), 6.81-6.77 (m, 1H), | MeOH/RT 1(1) |
| | | hexanamide | 4.57 (s, 2H), 4.02 (t, J=6.1 Hz, 2H), 3.94 (s, 2H), 3.46-3.42 (m, 4H), | |
| | | | 2.39 (br s, 4H), 1.99 (quintet, J=6.3 Hz, 2H), 1.57 (quintet, J=5.5 Hz, | |
| | | | 4H), 1.45-1.44 (m, 2H). | |
| | | | LRMS (ESI): (calc) 436.2; (found) 437.2 (MH)⁺. | |
| 312 | 512 | N-(biphenyl-3-yl)-6-(2- | (DMSOD6) δ (ppm): 9.96 (s, 1H), 8.28 (t, J=5.5 Hz, 1H), 7.91 (s, 1H), | 1 (6 (ex 2)) |
| | | (furan-2-ylmethylsulfinyl)- | 7.67-7.66 (m, 1H), 7.59-7.54 (m, 3H), 7.45 (t, J=7.2 Hz, 2H), | |
| | | acetamido)hexanamide | 7.38-7.33 (m, 2H), 7.30-7.28 (m, 1H), 6.46 (dd, J=3.1, 1.7 Hz, | |
| | | | 1H), 6.42 (d, J=2.7 Hz, 1H), 4.31 (d, J=14.1 Hz, 1H), 4.12 (d, | |
| | | | J=14.1 Hz, 1H), 3.68 (d, J=13.3 Hz, 1H), 3.50 (d, J=13.3 Hz, | |
| | | | 1H), 3.08 (q, J=6.8 Hz, 2H), 2.30 (t, J=7.2 Hz, 2H), 1.59 (quintet, | |
| | | | J=7.4 Hz, 2H), 1.43 (quintet, J=7.6 Hz, 2H), 1.33-1.27 (m, 2H). | |
| | | | LRMS (ESI): (calc) 452.2; (found) 453.4 (MH)⁺. | |
| 313 | 513 | 2-(4-fluorobenzyloxy)-N-(2- | (DMSOD6) δ (ppm): 8.40-8.39 (m, 2H), 7.88 (t, J=6.1 Hz, | 1 (1) |
| | | (pyridin-3-yl)ethyl)acetamide | 1H), 7.61 (dt, J=7.6, 2.2 Hz, 1H), 7.39-7.35 (m, 2H), | |
| | | | 7.30-7.27 (dd, J=7.6, 4.7 Hz, 1H), 7.17 (t, J=9.0 Hz, | |
| | | | 2H), 4.44 (s, 2H), 3.83 (s, 2H), 3.35 (q, J=7.0 Hz, 2H), | |
| | | | 2.76 (t, J=7.0 Hz, 2H), 2.49-2.47 (m, 1H). | |
| | | | LRMS (ESI): (calc) 288.1; (found) 289.1 (MH)⁺. | |
| 314 | 514 | 2-(4-fluorobenzyloxy)-N-(2- | (DMSOD6) δ (ppm): 8.56 (d, J=5.3 Hz, 1H), 8.18 | 6 (1 (Ex 56)) |
| | | (4-(4-methoxyphenyl)pyrimidin- | (d, J=9.0 Hz, 2H), 8.08 (t, J=5.3 Hz, 1H), 7.69 (d, | DMF |
| | | 2-ylthio)ethyl)acetamide | J=5.3 Hz, 1H), 7.38 (m, 2H), 7.15 (t, J=9.0 Hz, 2H), | 16 (5) EtOH |
| | | | 7.05 (d, J=9.0 Hz, 2H), 4.47 (s, 2H), 3.87 (s, 2H), | 1(1) |
| | | | 3.82 (s, 3H), 3.52-3.47 (m, 2H), 3.38-3.28 (m, 2H). | |
| | | | LRMS (ESI): (calc) 427.1; (found) 428.6 (MH)⁺. | |
| 315 | 515 | 2-(4-fluorobenzyloxy)-N-(2- | (DMSOD6) δ (ppm): 8.56 (d, J=5.3 Hz, 1H), 8.07-8.06 | 6 (1 (Ex 56)) |
| | | (4-(thiophen-2-yl)pyrimidin- | (m, 1H), 8.05 (t, J=5.7 Hz, 1H), 7.84 (d, J=4.9 Hz, 1H), | DMF |
| | | 2-ylthio)ethyl)acetamide | 7.65 (d, J=5.3 Hz, 1H), 7.40-7.37 (m, 2H), 7.25-7.23 (m, 1H), | 16 (5) EtOH |
| | | | 7.15 (t, J=8.8 Hz, 2H), 4.47 (s, 2H), 3.87 (s, 2H), 3.48 (q, | 1 (1) |
| | | | J=6.5, 12.5 Hz, 2H), 3.29-3.25 (m, 2H). | |
| | | | LRMS (ESI): (calc) 403.1; (found) 404.1 (MH)⁺. | |
| 316 | 516 | 2-(4-fluorobenzyloxy)-N-(2- | (MeOD-d4) δ (ppm): 8.80 (d, J=5.3 Hz, 1H), 8.23 (d, | 1 (6 (ex 2)) |
| | | (4-(4-methoxyphenyl)pyrimidin- | J=8.6 Hz, 2H), 7.95 (d, J=5.5 Hz, 1H), 7.33 (dd, J=8.6, | |
| | | 2-ylsulfinyl)ethyl)acetamide | 5.7 Hz, 2H), 7.08-7.02 (m, 4H), 4.45 (s, 2H), | |
| | | | 3.88 (s, 3H), 3.83-3.70 (m, 4H), 3.60-3.43 (m, 2H). | |
| | | | LRMS (ESI): (calc) 443.1; (found) 444.5 (MH)⁺. | |
| 317 | 517 | 2-(4-fluorobenzyloxy)-N-(4- | (MeOD-d4) δ (ppm): 8.43 (d, J=5.3 Hz, 1H), 8.11 (d, | 6 (1 (Ex 56)) |
| | | (4-(4-methoxyphenyl)pyrimidin- | J=8.0 Hz, 2H), 7.49 (d, J=5.3 Hz, 1H), 7.34-7.30 | DMF |
| | | 2-ylthio)butyl)acetamide | (m, 2H), 7.06-7.00 (m, 4H), 4.47 (s, 2H), 3.87 (s, 2H), | 16 (5) EtOH |
| | | | 3.86 (s, 3H), 3.34-3.28 (m, 2H), 3.24 (t, J=7.0 Hz, 2H), | 1 (1) |
| | | | 1.84-1.79 (m, 2H), 1.75-1.69 (m, 2H). | |
| | | | LRMS (ESI): (calc) 455.2; (found) 456.2 (MH)⁺. | |
| 318 | 518 | 8-(2-(2-aminophenyl)hydrazinyl)- | (MeOD-d4) δ (ppm): 7.84 (t, J=1.6 Hz, 1H), 7.61-7.58 | 1 (1) |
| | | N-(biphenyl-3-yl)-8-oxoctanamide | (m, 2H), 7.53 (dt, J=7.4, 2.0 Hz, 1H), 7.44-7.30 (m, 5H), | With Molecular |
| | | | 6.79-6.61 (m, 4H), 2.40 (t, J=7.2 Hz, 2H), 2.28 (t, J=7.2 | Sieves |
| | | | Hz, 2H), 1.76-1.68 (m, 4H), 1.46-1.42 (m, 4H). | 5 (3, ex 55) |
| | | | LRMS (ESI): (calc) 430.2; (found) 431.2 (MH)⁺. | |
| 319 | 519 | N-(biphenyl-3-yl)-8-oxo-8-(2- | (MeOD-d4) δ (ppm): 7.85 (t, J=1.8 Hz, 1H), 7.61-7.58 | 1 (1) |
| | | phenylhydrazinyl)octanamide | (m, 2H), 7.53 (dt, J=7.5, 2.0 Hz, 1H), 7.45-7.31 (m, 5H), | |
| | | | 7.16 (t, J=7.6 Hz, 2H), 6.79-6.76 (m, 3H), 2.41 (t, | |
| | | | J=7.2 Hz, 2H), 2.28 (t, J=7.2 Hz, 2H), 1.72 (quintet, | |
| | | | J=7.6 Hz, 4H), 1.45 (quintet, J=3.5 Hz, 4H). | |
| | | | LRMS (ESI): (calc) 415.2; (found) 416.2 (MH)⁺. | |
| 320 | 520 | 6-(2-(allyloxy)acetamido)-N- | (MeOD-d4) δ (ppm): 7.85 (t, J=1.7 Hz, 1H), 7.59-7.51 | 1 (1) |
| | | (biphenyl-3-yl)hexanamide | (m, 2H), 7.53 (dt, J=7.5, 1.8 Hz, 1H), 7.42-7.28 (m, 5H), | Pyridine |
| | | | 5.93-5.83 (m, 1H), 5.30-5.24 (m, 1H), 5.19-5.15 (m, 1H), | |
| | | | 4.01-3.98 (m, 2H), 3.23 (t, J=7.0 Hz, 2H), 2.38 (t, J=7.3 | |
| | | | Hz, 2H), 1.72 (quintet, J=7.5 Hz, 2H), 1.56 (quintet, J=7.5 | |
| | | | Hz, 2H), 1.43-1.36 (m, 2H). | |
| | | | LRMS (ESI): (calc) 380.2; (found) 381.2 (MH)⁺. | |
| 321 | 521 | methyl 3-(2-(6-(biphenyl-3- | (MeOD-d4) δ (ppm): 7.85 (t, J=1.8 Hz, 1H), 7.60-7.56 | 1 (4 (Ex 1)) |
| | | ylamino)-6-oxohexylamino)- | (m, 2H), 7.52 (dt, J=7.2, 2.0 Hz, 1H), 7.43-7.20 (m, 10H), | 70° C. |
| | | 2-oxoethylthio)-3- | 4.32 (t, J=7.9 Hz, 1H), 3.56 (s, 3H), 3.17-3.13 (m, 2H), | 1 (1) |
| | | phenylpropanoate | 3.09-2.81 (m, 4H), 2.41 (t, J=7.2 Hz, 2H), 1.74 (quintet, | |
| | | | J=7.4 Hz, 2H), 1.56-1.50 (m, 2H), 1.45-1.39 (m, 2H). | |
| 322 | 522 | ethyl 3-(2-(6-(biphenyl-3- | (MeOD-d4) δ (ppm): 7.85 (t, J=1.6 Hz, 1H), 7.60-7.57 | 1 (4 (Ex 1)) |
| | | ylamino)-6-oxohexylamino)- | (m, 2H), 7.53 (dt, J=7.3, 2.0 Hz, 1H), 7.44-7.20 (m, 10H), | 70° C. |
| | | 2-oxoethylthio)-3- | 4.32 (t, J=8.4 Hz, 1H), 4.04-3.98 (m, 2H), 3.18-2.78 (m, 6H), | 1 (1) |
| | | phenylpropanoate | 2.42 (t, J=7.2 Hz, 2H), 1.74 (quintet, J=7.6 Hz, 2H), 1.55 (quintet, | |
| | | | J=7.2 Hz, 2H), 1.46-1.40 (m, 2H), 1.11 (t, J=7.0 Hz, 3H). | |
| | | | LRMS (ESI): (calc) 532.3; (found) 533.3 (MH)⁺. | |
| 323 | 523 | ethyl 3-(2-(6-(biphenyl-3- | (MeOD-d4) δ (ppm): 7.84(t, J=1.8 Hz, 1H), 7.61-7.58 | 1 (6 (ex 2)) |
| | | ylamino)-6-oxohexylamino)- | (m, 2H), 7.54-7.48 (m, 2H), 7.44-7.30 (m, 9H), 5.07, | |
| | | 2-oxoethylsulfinyl)-3- | 5.27 (s, s, 1H), 4.30-4.19 (m, 2H), 3.89-3.38 (m, 2H), | |
| | | phenylpropanoate | 3.20 (q, J=6.8 Hz, 2H), 2.38 (td, J=7.5, 1.4 Hz, 2H), | |
| | | | 1.75-1.67 (m, 2H) 1.57-1.50 (m, 2H) 1.44-1.38 (m, 2H), | |
| | | | 1.26-1.27 (m, 3H). | |
| | | | LRMS (ESI): (calc) 534.2; (found) 535.2 (MH)⁺. | |
| 324 | 524 | ethyl 2-(2-oxo-2-(5-(5- | (MeOD-d4) δ (ppm): 7.94-7.90 (m, 3H), 7.55-7.47 (m, 3H), | 1 (1) |
| | | phenyl-1,3,4-thiadiazol-2- | 7.43-7.40 (m, 2H), 7.35-7.26 (m, 3H), 4.83 (s, 1H), 4.20-4.08 | |
| | | yl)pentylamino)ethylthio)-2- | (m, 2H), 3.23-3.05 (m, 6H), 1.86 (quintet, J=7.8 Hz, 2H), | |
| | | phenylacetate | 1.59-1.53 (m, 2H), 1.49-1.41 (m, 2H), 1.19 (t, J=7.0 Hz, 3H). | |
| | | | LRMS (ESI): (calc) 483.2; (found) 484.3 (MH)⁺. | |
| 325 | 525 | ethyl 2-(2-oxo-2-(6-oxo-6- | (MeOD-d4) δ (ppm): 8.85 (dd, J=4.1, 1.6 Hz, 1H), 8.62 (dd, | 1 (1) |
| | | (quinolin-8-ylamino)- | J=7.6, 1.2 Hz, 1H), 8.29 (dd, J=8.4, 1.7 Hz, 1H), 7.60 (dd, | |
| | | hexylamino)ethylthio)-2- | J=8.2, 1.2 Hz, 1H), 7.55-7.50 (m, 2H), 7.43-7.40 (m, 2H), | |
| | | phenylacetate | 7.34-7.30 (m, 3H), 4.82 (s, 1H), 4.18-4.09 (m, 2H), 3.221∝3.04 | |
| | | | (m, 4H), 2.60 (t, J=7.4 Hz, 2H), 1.79 (quintet, J=7.4 Hz, 2H), | |
| | | | 1.58-1.52 (m, 2H), 1.49-1.42 (m, 2H), 1.18 (t, J=7.2 Hz, 3H). | |
| | | | LRMS (ESI): (calc) 493.2; (found) 494.3 (MH)⁺. | |
| 326 | 526 | N-(3-(4-(3,4-dimethoxybenzyl- | (DMSOD6) δ (ppm): 7.89 (t, J=5.7 Hz, 1H), 7.57 | 31 (1 & 2) |
| | | sulfonyl)phenyl)propyl)-2-(4- | (d, J=8.4 Hz, 2H), 7.44-7.38 (m, 4H), 7.12 (t, J=9.0 Hz, | THF/thiol |
| | | fluorobenzyloxy)acetamide | 2H), 6.84 (d, J=8.4 Hz, 2H), 6.68 (dd, J=8.4, 2.1 Hz, 1H), | 1-pot RT |
| | | | 6.53 (d, J=2.0 Hz, 1H), 4.50 (d, J=5.8 Hz, 2H), | 1 (5 (ex 3)) |
| | | | 3.87 (s, 2H), 3.69 (s, 3H), 3.51 (s, 3H), 3.10 (q, J=6.8 Hz, | 25 (2 (ex 85a)) |
| | | | 2H), 2.64 (t, J=7.4 Hz, 2H), 1.72 (quintet, J=7.4 Hz, 2H). | 5 (3 (ex 55)) |
| | | | LRMS (ESI): (calc) 515.2; (found) 516.2 (MH)⁺. | |
| 327 | 527 | ethyl 2-(2-(3-(4-(N-(3,4- | (MeOD-d4) d (ppm): 8.54 (s, 1H), 7.60 (d, J=8.2 Hz, 2H), | 16 (5) ETOH |
| | | dimethoxyphenyl)sulfamoyl) | 7.43-7.39 (m, 2H), 7.33-7.24 (m, 5H), 6.76 (d, J=8.6 | 1 (1) |
| | | phenyl)propylamino)-2- | Hz, 1H), 6.67 (d, J=2.3 Hz, 1H), 6.56 (dd, J=8.0, | |
| | | oxoethylthio)-2-phenylacetate | 2.3 Hz, 1H), 4.83 (s, 1H), 4.17-4.09 (m, 2H), 3.73 (s, 3H), | |
| | | | 3.69 (s, 3H), 3.22-3.05 (m, 4H), 2.67 (t, J=7.5 Hz, 2H), | |
| | | | 1.76 (quintet, J=7.7 Hz, 2H), 1.17 (t, J=7.0 Hz, 3H). | |
| | | | LRMS (ESI): (calc) 586.2; (found) 587.3 (MH)⁺. | |
| 328 | 528 | ethyl 2-(2-(3-(4-(N-(3,4- | (MeOD-d4) d (ppm): 8.54 (s, 1H), 7.60 (d, J=8.2 Hz, 2H), | 1 (4 (Ex 1)) |
| | | dimethoxyphenyl)sulfamoyl) | 7.43-7.39 (m, 2H), 7.33-7.24 (m, 5H), 6.76 (d, | 1 (5 (Ex 3)) |
| | | phenyl)propylamino)-2- | J=8.6 Hz, 1H), 6.67 (d, J=2.3 Hz, 1H), 6.56 (dd, | 25 (2 (Ex 84a)) |
| | | oxoethylthio)-2-phenylacetate | J=8.0, 2.3 Hz, 1H), 4.83 (s, 1H), 4.17-4.09 (m, 2H), | 5 (3) (ex 55) |
| | | | 3.73 (s, 3H), 3.69 (s, 3H), 3.22-3.05 (m, 4H), 2.67 | |
| | | | (t, J=7.5 Hz, 2H), 1.76 (quintet, J=7.7 Hz, 2H), | |
| | | | 1.17 (t, J=7.0 Hz, 3H). | |
| | | | LRMS (ESI): (calc) 515.2; (found) 516.1 (MH)⁺. | |
| 329 | 529 | N-(3-(4-(N-(3,4-dimethoxy- | (MeOD-d4) d (ppm): 7.71 (dt, J=5.8, 1.2 Hz, 1H), 7.62-7.56 | 25 (2 (Ex 84a)) |
| | | phenyl)sulfamoyl)phenyl)propyl)- | (m, 3H), 7.47-7.42 (m, 2H), 7.37-7.29 (m, 3H), 6.76 | 5 (3) (ex 55) |
| | | benzofuran-2-carboxamide | (d, J=8.6 Hz, 1H), 6.65 (d, J=2.4 Hz, 1H), 6.55 (dd, | 1 (1) |
| | | | J=8.6, 2.3 Hz, 1H), 3.73 (s, 3H), 3.69 (s, 3H), 3.41 (t, | |
| | | | J=7.0 Hz, 2H), 2.75 (t, J=7.6 Hz, 2H), 1.94 (quintet, | |
| | | | J=7.4 Hz, 2H). | |
| | | | LRMS (ESI): (calc) 494.2; (found) 495.2 (MH)⁺. | |
| 330 | 530 | N-(3-(3-(((2-(1H-indol-3- | (MeOD-d4) d (ppm): 7.39-7.28 (m, 4H), 7.16 (t, J=7.6 Hz, | 32 (1) |
| | | yl)ethyl)(2-hydroxyethyl)amino)- | 1H), 7.06-6.97 (m, 4H), 6.94-6.89 (m, 3H), 6.76-6.73 | 27 (5 (Ex 87a)) |
| | | methyl) phenoxy)propyl)-2-(4- | (m, 1H), 4.49 (s, 2H), 3.93 (t, J=5.9 Hz, 2H), 3.89 (s, 2H), | 27 (1 (Ex 88b)) |
| | | fluorobenzyloxy)acetamide | 3.69 (s, 2H), 3.61 (t, J=6.2 Hz, 2H), 3.41 (t, J=6.6 Hz, | 16 (5) ETOH |
| | | | 2H), 2.94-2.90 (m, 2H), 2.83-2.79 (m, 2H), 2.72 (t, 1 (1) | |
| | | | J=6.3 Hz, 2H), 1.95 (quintet, J=6.3 Hz, 2H). | |
| | | | LRMS (ESI): (calc) 533.3; (found) 534.4 (MH)⁺. | |
| 331 | 531 | 2-(4-aminophenylthio)-N- | (MeOD-d4) d (ppm) ¹H: 7.89 (s, 1H), 7.60-7.57 (m, 2H), | 23 |
| | | (5-(5-phenylthiazol-2- | 7.42-7.38 (m, 2H), 7.35-7.30 (m, 1H), 7.22-7.18 (m, 2H), | (1-4 (ex 82)) |
| | | yl)pentyl)acetamide | 6.65-6.61 (m, 2H), 3.35 (s, 2H), 3.13 (t, J=6.8 Hz, 2H), | 1 (step 2) |
| | | | 2.99 (t, J=7.6 Hz, 2H), 1.81-1.74 (m, 2H), 1.49-1.42 | Ex 1 |
| | | | (m, 2H), 1.34-1.26 (m, 2H) | |
| | | | LRMS (ESI): (calc) 411.2 (found) 412.69 (MH)⁺ | |
| 332 | 532 | 2-(4-aminophenylthio)-N-(5- | (MeOH-d4) δ (ppm) ¹H: 7.76 (s, 1H), 7.51 (d, J=9.0 | 23 |
| | | (5-(4-methoxyphenyl)thiazol- | Hz, 2H), 7.19 (d, J=8.4 Hz, 2H), 6.96 (d, J=9.0 Hz, 2H), | (steps 1-4) |
| | | 2-yl)pentyl)acetamide | 6.63 (d, J=8.4 Hz, 2H), 3.82 (s, 3H), 3.34 (s, 2H), 3.13 | Ex 82 |
| | | | (t, J=6.8 Hz, 2H), 2.97 (t, J=7.6 Hz, 2H), 1.80-1.73 | (step 2) |
| | | | (m, 2H), 1.49-1.41 (m, 2H), 1.34-1.26 (m, 2H) | Ex 1 |
| | | | LRMS (ESI): (calc) 441.15 (found) 442.36 (MH)⁺ | |
| 333 | 533 | 2-(4-fluorobenzyloxy)-N-(5- | (MeOH-d4) δ (ppm) ¹H: 7.94-7.92 (m, 2H), 7.54-7.49 | 23 |
| | | (5-phenyl-1,3,4-thiadiazol-2- | (m, 3H), 7.39-7.36 (m, 2H), 7.08-7.04 (t, J=8.8 Hz, 2H), | (steps 1-3) |
| | | yl)pentyl)acetamide | 4.54 (s, 2H), 3.30-3.23 (t, J=6.8 Hz, 2H), 3.18-3.14 (t, | Ex 82 |
| | | | J=7.4 Hz, 2H), 1.91-1.84 (m, 2H), 1.63-1.56 (m, 2H), | 11 (step 4) |
| | | | 1.49-1.43 (m, 2H) | |
| | | | LRMS (ESI): (calc) 413.16 (found) 414.21 (MH)⁺ | |
| 334 | 534 | N-(4-(5-(3,4-dimethoxy- | (MeOH-d4) δ (ppm) ¹H: 7.50-7.33 (m, 4H), 7.10-7.03 | 22 |
| | | phenylsulfonamido)-1,3,4- | (m, 3H), 4.89 (s, 2H), 3.92 (s, 2H), 3.87 (s, 3H), 3.85 (s, 3H), | (Steps 1-4) |
| | | thiadiazol-2-ylthio)butyl)- | 3.25 (t, J=6.7 Hz, 2H), 3.18 (t, J=7.1 Hz, 2H), 1.76-1.62 | Ex 81a |
| | | 2-(4-fluorobenzyloxy)acetamide | (m, 4H) LRMS (ESI): (calc) 570.1 (found) 571.1 (MH)⁺ | |
| 335 | 535 | N-(4-(3-((3-(3,4-dimethoxy- | (MeOD-d4) δ (ppm) ¹H: 7.43-7.38 (m, 3H), 7.31 (d, | 32 (1-4) |
| | | phenylsulfonamido)pyrrolidin-1- | J=2.2 Hz, 1H), 7.17 (t, J=7.8 Hz, 1H), 7.10-7.02 | 1 (5 (ex 5)) |
| | | yl)methyl)phenoxy)butyl)-2- | (m, 3H), 6.82-6.78 (m, 3H), 4.56 (s, 2H), 3.94 (s, 2H), | 16 (1) |
| | | (4-fluorobenzyloxy)acetamide | 3.88 (s, 3H), 3.84 (s, 3H), 3.75-3.68 (m, 1H), 3.52 (d, | |
| | | | J=12.5 Hz, 1H), 3.45 (d, J=12.7 Hz, 1H), 3.35-3.30 | |
| | | | (m, 2H), 2.59-2.46 (m, 3), 2.18 (dd, J=10.0, 5.9 Hz, 1H), | |
| | | | 2.09-1.99 (m, 1H), 1.82-1.67 (m, 4H), 1.58-1.50(m, 1H) | |
| | | | LRMS (ESI): (calc) 629.3 (found) 630.88 (MH)⁺. | |
| 336 | 536 | pyridin-3-ylmethyl 1-(3-(4-(2- | (MeOH-d4) δ (ppm) ¹H: 8.54 (s, 1H), 8.47 (d, | 32 (1-4) |
| | | (4-fluorobenzyloxy)acetamido) | J=3.9 Hz, 1H), 7.84 (d, J=7.2 Hz, 1H), 7.44-7.38 | 1 (5 (ex 5)) |
| | | butoxy)benzyl)pyrrolidin-3- | (m, 3H), 7.20 (t, J=8.2 Hz, 1H), 7.10-7.05 (m, 2H), | 26 (Step 2) |
| | | ylcarbamate | 6.90-6.80 (m, 3H), 5.11 (s, 2H), 4.56 (s, 2H), 4.12-4.11 | Ex 81a |
| | | | (m, 1H), 3.99 (t, J=5.9 Hz, 2H), 3.93 (s, 2H), 3.57 (s, | |
| | | | 2H), 3.32-3.29 (m, 2H), 2.84-2.80 (m, 1H), 2.67-2.64 | |
| | | | (m, 1H), 2.56-2.51 (m, 1H), 2.40 (dd, J=14.7, | |
| | | | 8.0 Hz, 1H), 2.25-2.19 (m, 1H), 1.82-1.61 (m, 5H) | |
| | | | LRMS (ESI): (calc) 564.3 (found) 565.95 (MH⁺) | |
| 337 | 537 | N-(1-(3-(4-(2-(4- | (MeOH-d4) δ (ppm) ¹H: 8.68-8.67 (m, 2H), 7.77-7.76 | 32 (1-4) |
| | | fluorobenzyloxy)acetamido) | (m, 2H), 7.42-7.38 (m, 2H), 7.22 (t, J=8.0 Hz, 1H), 7.10-7.05 | 1 (5 (ex 5)) |
| | | butoxy)benzyl)pyrrolidin-3- | (m, 2H), 6.93-6.90 (m, 2H), 6.82 (dd, J=8.2, 2.3 Hz, 1H), | 1 (step 3) |
| | | yl)isonicotinamide | 4.56-4.53 (m, 3H), 3.99 (t, J=6.1 Hz, 2H), 3.93 (s, 2H), 3.63 | Ex 1 |
| | | | (s, 2H), 3.32-3.29 (m, 2H), 2.91-2.87 (m, 1H), 2.84-2.78 (m, | |
| | | | 1H), 2.62-2.54 (m, 2H), 2.40-2.31 (m, 1H), 1.87-1.65 (m, 5H) | |
| | | | LRMS (ESI): (calc) 534.3 (found) 535.77 (MH)⁺ | |
| 338 | 538 | 1-(3-(4-(2-(4-fluoro- | (MeOH-d4) δ (ppm) ¹H: 8.47-8.42 (m, 2H), 7.75 (d, | 32 (1-4) |
| | | benzyloxy)acetamido) | J=7.8 Hz, 1H), 7.42-7.37 (m, 3H), 7.20 (t, J=7.8 Hz, 1H), | 1 (5 (ex 5)) |
| | | butoxy)benzyl)pyrrolidin-3-yl | 7.10-7.05 (m, 2H), 6.91-6.87 (m, 2H), 6.82 (ddd, J=8.2, | 26 (step 1) |
| | | pyridin-3-ylmethylcarbamate | 2.5, 0.8 Hz, 1H), 5.10-5.06 (m, 1H), 4.56 (s, 2H), 4.30 | Ex 86a |
| | | | (s, 2H), 3.99 (d, J=5.9 Hz, 2H), 3.93 (s, 2H), 3.65 (d, | 16 (Step 5) |
| | | | J=12.7 Hz, 1H), 3.53 (d, J=12.7 Hz, 1H), 3.33-3.29 | Ex 73a |
| | | | (m, 2H), 2.83-2.63 (m, 3H), 2.48-2.42 (m, 1H), 2.27-2.22 | 1 (Step 1) |
| | | | (m, 1H), 1.88-1.68 (m, 5H) | Ex 1 |
| | | | LRMS (ESI): (calc) 564.3 (found) 565.61 (MH)⁺ | |
| 339 | 539 | (E)-N-(3-(4-(((2-(1H-indol-3- | (MeOH-d4) δ (ppm) ¹H: 7.44-7.28 (m, 8H), | 11 (step 1) |
| | | yl)ethyl)(2-hydroxyethyl)- | 7.10-6.98 (m, 4H), 6.91 (t, J=7.8 Hz, 1H), 6.53 (d, | Ex 66 |
| | | amino)methyl) phenyl)allyl)- | J=16.0 Hz, 1H), 6.21 (dt, J=16.0, 5.9 Hz, 1H), 4.60 | 27 (step 1) |
| | | 2-(4-fluorobenzyloxy)acetamide | (s, 2H), 4.01-3.99 (m, 4H), 3.73 (s, 2H), 3.63 (t, | Ex 88b |
| | | | J=6.3 Hz, 2H), 2.95-2.92 (m, 2H), 2.84-2.80 (m, | 25 (step 2) |
| | | | 2H), 2.74 (t, J=6.1 Hz, 2H) | Ex 85a |
| | | | LRMS (ESI): (calc) 515.3 (found) 516.2 (MH)⁺ | 16 (step 5) |
| | | | | Ex 73a |
| | | | | 1 (step 1) |
| | | | | Ex 1 |
| 340 | 540 | (S)-benzyl 1-oxo-6-(2- | (MeOD-d4) δ (ppm) ¹H: 8.75 (dd, J=4.1, 1.6 Hz, 1H), | 43 (1-2) |
| | | (pyridin-4-ylthio)acetamido)- | 8.64 (d, J=6.8 Hz, 1H), 8.37-8.25 (m, 3H), 7.62 (d, | 1 (3 (ex 1)) |
| | | 1-(quinolin-8-ylamino)- | J=8.3 Hz, 1H), 7.56-7.5 (m, 2H), 7.4-7.2 (m, 6H), | 1 (4 (ex 1)) |
| | | hexan-2-ylcarbamate | 5.22 and 5.11 (ab doublet, J=2.1 Hz, 2H), 4.38-4.2 | DIPEA at 50C |
| | | | (m, 1H), 3.78 (s, 2H), 3.23 (t, J=6.4 Hz, 2H), 2.1-1.95 | |
| | | | (m, 1H), 1.85-1.75 (m, 1H), 1.6-1.4 (m, 4H). | |
| | | | LRMS (ESI): (calc) 557.2 (found) 558.8 (MH)⁺. | |
| 341 | 541 | (R)-benzyl 1-oxo-6-(2- | (MeOD-d4) δ (ppm) ¹H: 8.74 (dd, J=4.2, 1.6 Hz, 1H), | 43 (1-2) |
| | | (pyridin-4-ylthio)acetamido)- | 8.64 (d, J=6.8 Hz, 1H), 8.37-8.2 (m, 4H), 7.65-7.6 | 1 (3 (ex 1)) |
| | | 1-(quinolin-8-ylamino)hexan- | (m, 1H), 7.55-7.5 (m, 2H), 7.39 (dd, J=5.3, 1.6 Hz, 2H), | 1 (4 (ex 1)) |
| | | 2-ylcarbamate | 7.3-7.25 (m, 4H), 5.22 and 5.11 (ab doublet, J=2.5 Hz, 2H), | DIPEA at 50C |
| | | | 4.34-4.30 (m, 1H), 3.76 (s, 2H), 3.22 (t, J=6.6 Hz, 2H), | |
| | | | 2.05-1.95 (m, 1H), 1.81-1.75 (m, 1H), 1.6-1.4 (m, 4H,). | |
| | | | LRMS (ESI): (calc) 557.2 (found) 558.4 (MH)⁺. | |
| 342 | 542 | (S)-benzyl 6-(2-(4-amino- | (MeOD-d4) δ (ppm) ¹H: 8.76 (dd, J=4.2, 1.6 Hz, 1H), | 43 (1-2) |
| | | phenylthio)acetamido)-1-oxo- | 8.62 (d, J=7.5 Hz, 1H), 8.29 (dd, J=8.2, 1.6 Hz, 1H), 7.62 | 1 (3 (ex 1)) |
| | | 1-(quinolin-8-ylamino)- | (d, J=8.4 Hz, 1H), 7.56-7.51 (m, 2H), 7.4-7.36 (m, 1.5H), | 1 (4 (ex 1)) |
| | | hexan-2-ylcarbamate | 7.3-7.25 (m, 2.5H), 7.19 (d, J=8.6 Hz, 2H), 6.63 (d, | DIPEA at 50C |
| | | | J=8.4 Hz, 2H), 5.21 and 5.11 (ab doublet, J=12.3 Hz, 2H), | |
| | | | 4.33-4.28 (m, 1H), 3.34 (s, 2H), 3.13 (t, J=6.5 Hz, 2H,), | |
| | | | 2.0-1.84 (m, 1H), 1.8-1.61 (m, 1H), 1.5-1.2 (m, 1H). | |
| | | | LRMS (ESI): (calc) 571.2; (found) 572.8 (MH)⁺. | |
| 343 | 543 | (R)-benzyl 6-(2-(4-amino- | (MeOD-d4) δ (ppm) ¹H: 8.76 (dd, J=4.2, 1.7 Hz, 1H), | 43 (1-2) |
| | | phenylthio)acetamido)-1- | 8.64 (d, J=7.4 Hz, 1H), 8.29 (dd, J=8.3, 1.5 Hz, 1H), | 1 (3 (ex 1)) |
| | | oxo-1-(quinolin-8-ylamino)- | 7.62 (d, J=8.3 Hz, 1H), 7.56-7.51 (m, 2H), 7.4-7.37 | 1 (4 (ex 1)) |
| | | hexan-2-ylcarbamate | (m, 1.5H), 7.3-7.20 (m, 2.5H), 7.19 (d, J=8.6 Hz, 2H), | DIPEA at 50C |
| | | | 6.63 (d, J=8.4 Hz, 2H), 5.21 and 5.11 (ab doublet, | |
| | | | J=12.7 Hz, 2H), 4.33-4.22 (m, 1H), 3.34 (s, 2H), | |
| | | | 3.13 (t, J=6.5 Hz, 2H), 2.0-1.84 (m, 1H), 1.8-1.71 | |
| | | | (m, 1H), 1.5-1.22 (m, 1H). | |
| | | | LRMS (ESI): (calc) 571.2 (found) 572.9 (MH)⁺. | |
| 344 | 544 | N-(10,11-dihydro-5H- | (CDCl3-d) δ (ppm) ¹H: 7.45-7.38 (m, 2H), 7.33-7.25 | 1 (3 (ex 1)) |
| | | dibenzo[a,a]cyclohepten- | (m, 2H), 7.24-7.11 (m, 4H), 7.10-7.02 (m, 2H), 6.53 (br s, 1H), | |
| | | 5-yl)-6-(2-(4-fluorobenzyloxy)- | 6.36-6.30 (m, 1H), 6.27-6.21 (m, 1H), 4.50 (s, 2H), 3.91 (s, 2H), | |
| | | acetamido)-hexanamide | 3.39-3.16 (m, 5H), 3.14-3.02 (m, 2H), 2.17 (t, J=7.4 Hz, | |
| | | | 2H), 1.71-1.58 (m, 2H), 1.52-1.40 (m, 2H), 1.32-1.20 (m, 2H). | |
| | | | LRMS (ESI): (calc) 488.2 (found) 489.3 (MH)⁺. | |
| 345 | 545 | (S, E/Z (7:3))-N-(4-(2,5-dioxo- | (MeOD-d4) δ (ppm) ¹H: 8.66 (dd, J=2.4 hHz, 0.6H), | 2 (1 (ex 47)) |
| | | 14-phenyl-1,2,3,4,5,6,7,10- | 8.40 (dd, J=1.8 Hz, 0.3H), 7.57-7.54 (m, 2H), 7.45-7.25 | 43 (2 (ex 105a)) |
| | | octahydrobenzo[b][1,4,7]- | (m, 7H), 7.22 (d, J=8.4 Hz, 1H), 7.09-7.02 (m, 2H), | 1 (1 (ex 1)) |
| | | oxadiazacyclotridecin-3-yl)butyl)- | 6.12-6.02 (m, 0.7H), 5.78-5.65 (m, 1.3H), 4.68-4.42 | 1 (5 (ex 1)) |
| | | 2-(4-fluorobenzyloxy)acetamide | (m, 4H), 4.25-4.15 (m, 2H), 3.94-3.89 (m, 2H), 2.5-2.38 | 46 (1,2 (ex 108)) |
| | | | (m, 4H), 2.05-1.65 (m, 2H), 1.64-1.38 (m, 4H). | 45 (4 (ex 107)) |
| | | | LRMS (ESI): (calc) 573.3 (found) 574.2 (MH)⁺. | |
| 346 | 546 | (S)-N-(4-(2,5-dioxo-14-phenyl- | (MeOD-d4) δ (ppm) ¹H: 8.45 (d, J=2.2 Hz, 0.7H), | 5 (3 (ex 55)) |
| | | 1,2,3,4,5,6,7,8,9,10-decahydro- | 8.16 (s, 0.3H), 7.54 (d, J=7.5 Hz, 2H), 7.42-7.34 | |
| | | benzo[b][1,4,7]oxadiaza- | (m, 4H), 7.33-7.25 (m, 3H), 4.56-4.50 (m, 2H), | |
| | | cyclotridecin-3-yl)butyl)-2-(4- | 3.95-3.85 (m, 3H), 3.30-3.22 (m, 2H), 2.64-2.52 | |
| | | fluorobenzyloxy)acetamide | (m, 1H), 2.32-2.24 (m, 1H), 2.10-1.26 (m, 12H). | |
| | | | LRMS (ESI): (calc) 575.3 (found) 576.3 (MH)⁺. | |
| 347 | 547 | N-(3-(4-((3-(2-(1H-indol-3- | (acetone-d6) δ (ppm): 10.11 (bs, 1H): 7.58 (d, J=7.2 | 26 (1 (ex 86a)) |
| | | yl)ethyl)ureido)methyl)- | Hz, 1H); 7.45-7.27 (m, 4H); 7.18 (d, J=8.0 Hz, 2H); | inPy 26 |
| | | phenyl)propyl)-2-(4- | 7.14-7.05 (m, 5H); 7.01-6.96 (m, 1H); 4.58 (s), | (3 (ex 86a)) |
| | | fluorobenzyloxy)acetamide | 4.56 (s) (2H); 4.34 (s), 4.29 (s) (2H); 3.92 (s), 3.90 | 5 (3 (ex 55, |
| | | | (s)(2H); 3.47 (dd, J=6.8, 7.2 Hz, 2H); 3.23 (q, | comp74)), |
| | | | J=6.8 Hz, 2H); 2.91 (dd, J=6.8, 7.2 Hz, 2H); | in AcOEt |
| | | | 2.58 (t; J=7.6 Hz, 2H); 1.83-1.76 (m, 2H). | |
| 348 | 548 | 2-(1H-indol-3-yl)ethyl 4-(3- | (CDCl3) δ (ppm): 8.42 (bs, 1H); 7.62 (d, J=7.6 Hz, 1H); | 26 (1 (ex 86a)) |
| | | (2-(4-fluorobenzyloxy)acetamido) | 7.35-7.20 (m, 3H); 7.18-7.04 (m, 8H); 6.99 (bs, 1H); | in Py |
| | | propyl)benzylcarbamate | 6.61 (bs, 1H); 5.10 (bs, 1H); 4.51 (s, 2H); 4.38 (dd, | 26 (3 (ex 86a)) |
| | | | J=6.8, 7.2 Hz, 2H); 4.31 (bs, 2H); 3.96 (s, 2H); 3.31 | 5 (3 (ex 55, |
| | | | (q, J=6.8 Hz, 2H); 3.09 (dd, J=6.8, 7.2 Hz, 2H); | comp74)), |
| | | | 2.62 (t, J=7.6 hz, 2H); 1.87-1.80 (m, 2H). | in AcOEt |
| 349 | 549 | 2-(4-fluorobenzyloxy)-N-(3- | (MeOD-d4) δ (ppm): 7.39-7.35 (m, 3H); 7.30 (bd, | 26 (2 (ex 86a)) |
| | | (4-((2-(2-methyl-1H-indol-3- | J=8.4 Hz, 2H); 7.21 (bd, J=7.6 Hz, 1H); 7.15 (bd, | 27 (5 (ex 87a)) |
| | | yl)ethylamino)methyl)phenyl)- | J=8.0 Hz, 2H); 7.04 (bt, J=8.8 Hz, 2H); 6.98 (dt, | |
| | | prop-2-ynyl)acetamide | J=1.2, 8.0 Hz, 1H); 6.91 (dt, J=1.2, 8.0 Hz, 1H); | |
| | | | 4.54 (s, 2H); 4.21 (s, 2H); 3.95 (s, 2H); 3.69 (s, 2H); | |
| | | | 2.88 (t, J=7.2 Hz, 2H); 2.76 (t, J=7.2 Hz, 2H); | |
| | | | 2.31 (s, 3H). | |
| 350 | 550 | 2-(4-fluorobenzyloxy)-N-(3- | (MeOD-d4) δ (ppm): 7.39-7.36 (m, 3H); 7.21 (bd, | 5 (3(ex 55, |
| | | (4-((2-(2-methyl-1H-indol-3- | J=8.0 Hz, 1H); 7.14-7.04 (m, 6H); 6.98 (td, J=1.2, | comp74)), |
| | | yl)ethylamino)methyl)phenyl)- | 6.8 Hz, 1H); 6.91 (td, J=1.2, 8.0 Hz, 1H); 4.52 (s, 2H); | in AcOEt |
| | | propyl)acetamide | 3.90 (s, 2H); 3.71 (s, 2H); 3.22 (t, J=7.2 Hz, 2H); | |
| | | | 2.90 (t, J=6.8 Hz, 2H); 2.80 (d, J=6.8 Hz, 2H); | |
| | | | 2.58 (t, J=7.2 Hz, 2H); 2.32 (s, 3H); 1.78 (t, | |
| | | | J=7.2 Hz, 2H). | |
| 351 | 551 | (S)-N-(1-(5-chloro-6-fluoro- | (MeOD-d4) δ (ppm): 7.28 (t, J=5.5 Hz, 2H); 7.58 (d, | 1 (1) |
| | | 1H-benzo[d]imidazol-2-yl)- | J=6.7 Hz, 1H); 7.37 (m, 3H); 7.18 (t, J=9.8 Hz, 2H); | 1 (1) |
| | | 5-(2-(4-fluorophenylthio)- | 7.02 (t, J=8.8 Hz, 2H); 5.31 (t, J=9.6 Hz, 1H); | 48 (2) |
| | | acetamido) pentyl)-4- | 3.48 (s, 2H); 3.16 (t, J=6.7 Hz, 2H, J=6.7 Hz); | 1 (2) |
| | | fluorobenzamide | 2.11 (m, 2H); 1.51 (m, 2H); 1.38 (m, 2H) | 1 (1) |
| | | | LRMS (ESI): 560.3 (calc) 561.1 (MH)⁺. | |
| 352 | 552 | N-(5-(1H-benzo[d]imidazol- | (MeOD-d4) d (ppm): 7.55 (q, J=3.1, 6.1 Hz, 2H); 7.40 | 1 (1) |
| | | 2-yl)pentyl)-2-(4- | (m, 2H); 7.31 (q, J=3.1, 6.1 Hz, 2H); 7.02 (t, | 48 (2) |
| | | fluorophenylthio)acetamide | J=9.0 Hz, 2H); 3.50 (s, 2H): 3.14 (t, J=6.8 Hz, 2H); | 1 (2) |
| | | | 2.94 (t, J=7.4 Hz, 2H); 1.82 (m, 2H); 1.47 (m, 2H); | 1 (4, ex1) |
| | | | 1.29 (m, 2H) LRMS: 371.1 (calc) 372.5 (found) | |
| 353 | 553 | N,N-bis(2-(1H-indazol-5- | (MeOD-d4) d (ppm): 8.21 (s, 1H); 8.14 (s, 1H); 8.01 | 1 (1) |
| | | ylamino)-2-oxoethyl)-6- | (m, 2H); 7.57 (m, 4H); 7.31 (m, 2H); 7.01 (m, 2H); | 1 (5, ex1) |
| | | (2-(4-fluorobenzyloxy)- | 4.46 (m, 4H); 4.31 (s, 2H); 3.83 (s, 2H); 3.18 (m, | 1 (1) |
| | | acetamido) hexanamide | 2H); 2.47 (m, 2H); 1.65 (m, 2H); 1.52 LRMS (ESI): | |
| | | | (calc.) 628.2; (found) 629.3 (MH)⁺. | |
| 354 | 554 | N,N-bis(2-(1H-indazol-5- | (MeOD-d4) d (ppm): 8.22 (s, 1H); 8.14 (s, 1H); 8.01 | 1 (1) |
| | | ylamino)-2-oxoethyl)-5-(2- | (d, J=5.3 Hz, 2H); 7.53 (m, 4H); 7.30 (m, 2H); | 1 (5, ex1) |
| | | (4-fluorobenzyloxy)acetamido) | 7.03 (t, J=8.6 Hz, 2H); 4.44 (s, 2H); 4.43 (s, | 1 (1) |
| | | pentanamide | 2H); 4.30 (s, 2H); 3.82 (s, 2H); 3.14 (t, J=6.8 | |
| | | | Hz, 2H); 2.44 (t, J=7.2 Hz, 2H); 1.65 (m, 2H); | |
| | | | 1.45 (m, 2H); 1.31 (m, 2H) | |
| | | | LRMS (ESI): 642.6 (calc) 643.3 (found) | |

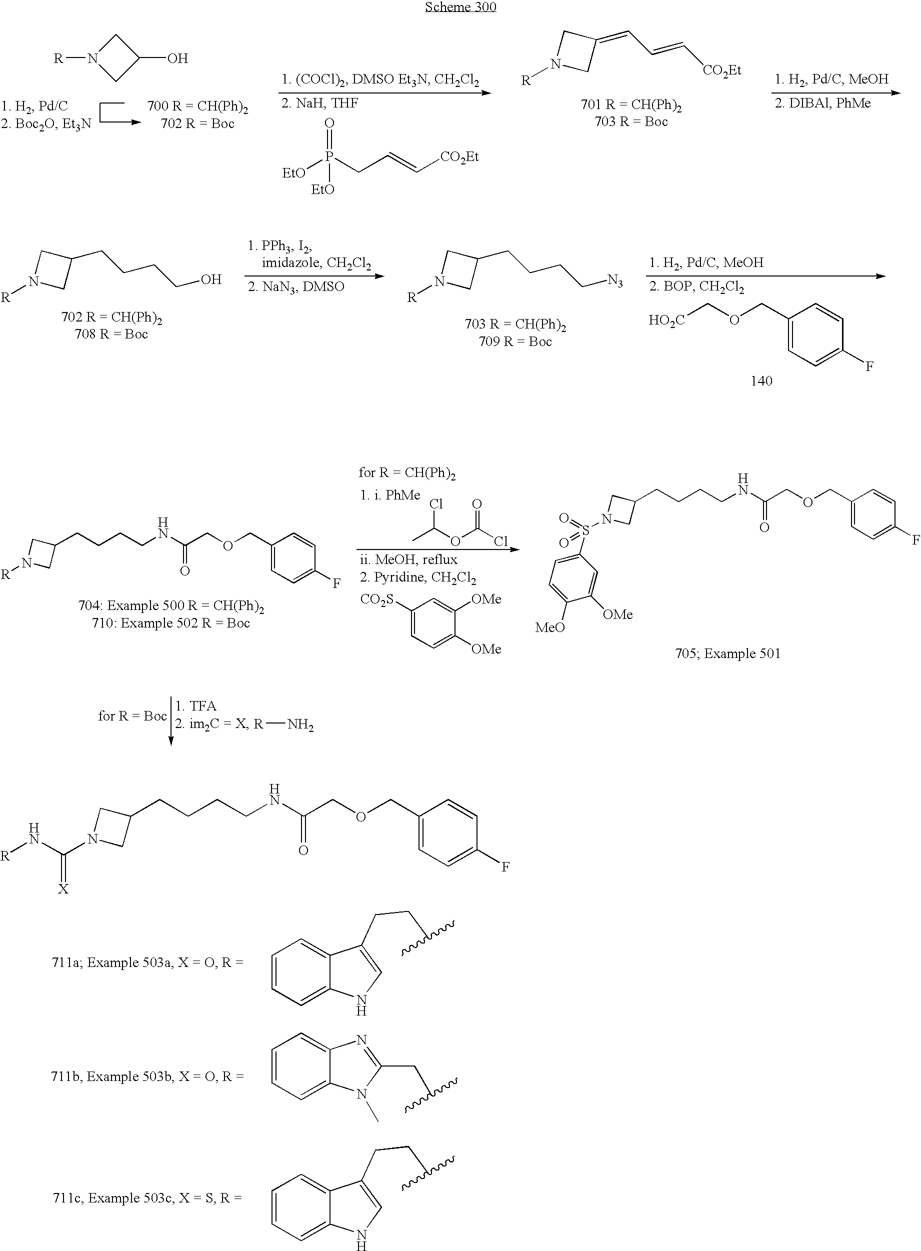

### EXAMPLE 500

### N-(4-(1-benzhydrylazetidin-3-yl)butyl)-2-(4-fluorobenzyloxy)acetamide (704)

### Step 1: (E)-ethyl 4-(1-benzhydrylazetidin-3-ylidene)but-2-enoate (701)

To a stirred solution of oxalyl chloride (0.91 mL, 10.46 mmol) in CH₂Cl₂ (20 mL) at −78° C. was added DMSO (0.74 mL, 10.46 mmol). After 20 min, a solution of 1-benzhydrylazetidin-3-ol 700 (1.0 g, 4.18 mmol) in CH₂Cl₂ (5 mL) was added. The solution was stirred at −78° C. for another 20 min and Et₃N (2.91 mL, 20.92 mmol) was added. The solution was slowly warmed to 0° C. over 1 h. A mixture of 1:1 EtOAc/hexane was added and the salt was filtered through a pad of Celite-Silica gel. The solution was evaporated to dryness and the crude ketone in THF (20 mL) was cooled in an ice bath. (E)-ethyl 4-(diethoxyphosphoryl)but-2-enoate (1.11 mL, 5.02 mmol) and NaH (218 mg, 5.44 mmol) were added and the resulting mixture was slowly warmed to room temperature and stirred for 1 h. H₂O was added and the mixture was extracted with EtOAc. The organic phase was dried (Na₂SO₄) and evaporated. The residue was purified by silica gel column chromatography with EtOAc (20%) in hexane to afford 701 (1.27 g, 99%) as a mixture of cis and trans isomers.

(CD₃CN) δ (ppm) ¹H: 7.49 (d, J=8.4 Hz, 4H), 7.31 (t, J=7.6 Hz, 4H), 7.22 (t, J=4.0 Hz, 2H), 7.12 (dd. J=4.0, 11.2 Hz, 1H), 6.06 (dt, J=2.0, 11.2 Hz, 1H), 5.82 (d, J=15.6 Hz, 1H), 4.62 (s, 1H), 4.13 (q, J=6.8 Hz, 2H), 3.98 (s, 2H), 3.84 (s, 2H), 1.24 (t, J=7.2 Hz, 3H). LRMS (ESI): (calc) 333.4; (found) 334.2 (MH)⁺.

### Step 2: 4-(1-benzhydrylazetidin-3-yl)butan-1-ol (702)

To a stirred solution of (E)-ethyl 4-(1-benzhydrylazetidin-3-ylidene)but-2-enoate 701 (965 mg, 2.89 mmol) in MeOH (29 mL) was carefully added palladium on charcoal (10% w/w) (30 mg). A balloon filled with hydrogen was attached to the flask and the suspension was stirred for 3 h. The suspension was filtered and evaporated. To a stirred solution of the resulting ester in PhMe (14 mL) at −78° C. was added a 1.0M solution of DIBAL in PhMe (13.9 mL, 13.94 mmol). After stirring for 1 h, MeOH was added and warmed to 0° C. 3 drops of H₂O were added followed by Et₂O (10 mL) and stirred at room temperature for 10 min. The gel that was formed was filtered through Celite and washed with EtOAc to yield 702 (522 mg, 60%). LRMS (ESI): (calc) 295.4; (found) 296.2 (MH)⁺.

### Step 3: 3-(4-azidobutyl)-1-benzhydrylazetidine (703)

To a stirred solution of 702 (522 mg, 1.77 mmol) in CH₂Cl₂ (4.5 mL) at 0° C. were added sequentially PPh₃ (509 mg, 1.94 mmol), imidazole (240 mg, 3.53 mmol) and a solution of iodine (494 mg, 1.94 mmol) in CH₂Cl₂ (10 mL). The yellow solution was stirred at room temperature for 1 h. Saturated solutions of NaHCO₃ and sodium thiosulfate were added and CH₂Cl₂ extractions. The organic phase was dried (Na₂SO₄) and evaporated. The residue was purified by silica gel column chromatography with EtOAc (15%) in hexane to afford 1-benzhydryl-3-(4-iodobutyl)azetidine (349 mg, 49%). LRMS (ESI): (calc) 405.3; (found) 406.4 (MH)⁺. To a stirred solution of 1-benzhydryl-3-(4-iodobutyl)azetidine (349 mg, 0.86 mmol) in DMSO (4.0 mL) was added NaN₃ (112 mg, 1.72 mmol). The solution was stirred for 0.5 h and H₂O was added followed by EtOAc extractions. The organic phase was dried over Na₂SO₄ and evaporated. The residue was purified by silica gel column chromatography with EtOAc (15%) in hexane to afford 703 (250 mg, 91%). LRMS (ESI): (calc) 320.4; (found) 321.2 (MH)⁺.

### Step 4: N-(4-(1-benzhydrylazetidin-3-yl)butyl)-2-(4-fluorobenzyloxy)acetamide (704)

To a stirred solution of 3-(4-azidobutyl)-1-benzhydrylazetidine 703 (250 mg, 0.78 mmol) in MeOH (5.0 mL) was carefully added palladium on charcoal (10% w/w) (20 mg). A balloon filled with hydrogen was attached to the flask and the suspension was stirred for 0.5 h. The suspension was filtered and evaporated. To a stirred solution of the amine in CH₂Cl₂ (7.5 mL) at 0° C. were added 2-(4-fluorobenzyloxy)acetic acid 140 (158 mg, 0.86 mmol), Et₃N (0.24 mL, 1.72 mmol) and BOP (380 mg, 0.86 mmol). The mixture was stirred at room temperature overnight and evaporated to dryness. The residue was purified by silica gel column chromatography with a gradient of EtOAc (50% to 70%) in hexane to afford 704 (243 mg, 68%).

(MEOD-d₄) δ (ppm) ¹H: 7.39-7.36 (m, 6H), 7.28-7.24 (m, 4H), 7.17 (t, J=7.6 Hz, 2H), 7.05 (t, J=8.4 Hz, 2H), 4.53 (s, 2H), 4.41 (s,1H), 3.90 (s, 2H), 3.36 (t, J=7.6 Hz, 2H), 3.19 (t, J=7.2 Hz, 2H), 2.76 (t, J=7.6 Hz, 2H), 2.44 (qt, J=7.2 Hz, 1H), 1.53 (qt, J=8.0 Hz, 2H), 1.47 (qt, J=7.6 Hz, 2H), 1.25-1.19 (m, 2H). LRMS (ESI): (calc) 460.6; (found) 461.3 (MH)⁺.

### EXAMPLE 501

### N-(4-(1-(3,4-dimethoxyphenysulfonyl)azetidin-3-yl)butyl)-2-(4-fluorobenzyloxy)acetamide (705)

To a stirred solution of N-(4-(1-benzhydrylazetidin-3-yl)butyl)-2-(4-fluorobenzyloxy)acetamide 704 (60 mg, 0.13 mmol) in ClCH₂CH₂Cl (0.9 mL) at 0° C. was added 1-chloroethyl chloroformate (0.03 mL, 0.30 mmol). The solution was warmed to room temperature and was then stirred overnight at 65° C. The solution was evaporated to dryness. The residue was dissolved in MeOH (1.3 mL) and heated at 70° C. for 6 h. The solution was evaporated to dryness to yield the crude amine. To a stirred solution of the amine in CH₂Cl₂ (0.9 mL) were added sequentially pyridine (0.02 mL, 0.20 mmol) and 3,4-dimethoxybenzene-1-sulfonyl chloride (40 mg, 0.17 mmol). The mixture was stirred at room temperature overnight and evaporated to dryness. The residue was purified by silica gel column chromatography with EtOAc (80%) in hexane to afford 705 (6.5 mg, 17%). (MEOD-d₄) δ (ppm) ¹H: 7.43 (dd, J=2.0, 8.4 Hz, 1H), 7.41-7.38 (m, 2H), 7.30 (d, J=2.0 Hz, 1H), 7.18 (d, J=8.8 Hz, 1H), 7.08 (t, J=8.8 Hz, 2H), 4.55 (s, 2H), 3.92 (s, 3H), 3.91 (s, 3H), 3.89 (s, 2H), 3.82 (t, J=8.4 Hz, 2H), 3.35 (t, J=6.0 Hz, 2H), 3.14 (t, J=7.2 Hz, 2H), 2.38 (qt, J=7.6 Hz, 1H), 1.39 (qt, J=7.2 Hz, 2H), 1.34-1.28 (m, 2H), 1.15-1.09 (m, 2H). LRMS (ESI): (calc) 494.6; (found) 495.2 (MH)⁺.

### EXAMPLE 502

### tert-butyl 3-(4-(2-(4-fluorobenzyloxy)acetamido)butyl)azetidine-1-carboxylate ((710)

### Step 1: tert-butyl 3-hydroxyazetidine-1-carboxylate (706)

To a stirred solution of 700 (1.0 g, 4.18 mmol) in MeOH (10 mL) was carefully added palladium on charcoal (10% w/w) (500 mg). A balloon filled with hydrogen was attached to the flask and the suspension was stirred for 2 h. The suspension was filtered and evaporated. To a stirred solution of the azetidin-3-ol in MeOH (20 mL) were added Et₃N (0.70 mL, 5.01 mmol) and Boc₂O (957 mg, 4.39 mmol). The resulting mixture was stirred at room temperature for 12 h. The solution was evaporated and purified by silica gel column chromatography with EtOAc (50%) in hexane to afford 706 (624 mg, 86%) as a clear oil.

(MEOD-d₄) δ (ppm) ¹H: 4.50-4.47 (m, 1H), 4.10 (t, J=7.2 Hz, 2H), 3.70 (dd, J=4.4, 9.2 Hz, 2H), 1.43 (s, 9H). LRMS (ESI): (calc) 173.2; (found) 212.0 (MK)⁺.

### Step 2: (E)-tert-butyl 3-(4-ethoxy-4-oxobut-2-enylidene)azetidine-1-carboxylate (707)

Following the same procedure as described for compound 701 but substituting 700 for carbamate 706, to afford 707 (385 mg, 40%).

(MEOD-d₄) δ (ppm) ¹H: 7.14 (dd, J=11.6, 15.2 Hz, 1H), 6.19 (d, J=11.6 Hz, 1H), 5.88 (d, J=15.2 Hz, 1H), 4.69 (s, 2H), 4.57 (s, 2H), 4.18 (q, J=6.8 Hz, 2H), 1.44 (s, 9H), 1.28 (t, J=7.2 Hz, 3H). LRMS (ESI): (calc) 267.3; (found) 290.0 (MNa)⁺.

### Step 3: tert-butyl 3-(4-hydroxybutyl)azetidine-1-carboxylate (708)

(E)-tert-butyl 3-(4-ethoxy-4-oxobut-2-enylidene)azetidine-1-carboxylate 707 (563 mg, 2.11 mmol) in MeOH (10.5 mL) and palladium on charcoal (10% w/w) (25 mg) was hydrogenated under 1 atm of hydrogen gas 3 h. The suspension was filtered and evaporated and the resulting ester in THF (10.5 mL) at 0° C. was added to LiAlH₄ (200 mg, 5.27 mmol). After 20 min, H₂O was added and the mixture was extracted with EtOAc. The organic phase was dried (Na₂SO₄) and evaporated to yield 708 (466 mg, 96%).

(CDCl₃) δ (ppm) ¹H: 3.92 (t, J=8.0 Hz, 2H), 3.58 (t, J=6.4 Hz, 2H), 3.46 (t, J=5.6 Hz, 2H), 2.45-2.38 (m, 1H), 1.88 (br, 1H), 1.57-1.46 (m, 4H), 1.36 (s, 9H), 1.29-1.17 (m, 2H). LRMS (ESI): (calc) 229.3; (found) 252.1 (MNa)⁺.

### Step 4: tert-butyl 3-(4-azidobutyl)azetidine-1-carboxylate (709)

To a stirred solution of tert-butyl 3-(4-hydroxybutyl)azetidine-1-carboxylate 708 (298 mg, 1.30 mmol) in CH₂Cl₂ (3.5 mL) at 0° C. were added sequentially PPh₃ (375 mg, 1.43 mmol), imidazole (177 mg, 2.60 mmol) and a solution of iodine (363 mg, 1.43 mmol) in CH₂Cl₂ (8.5 mL). The yellow solution was stirred at room temperature for 3 h. Saturated solutions of NaHCO₃ and sodium thiosulfate were added and CH₂Cl₂ extractions. The organic phase was dried over Na₂SO₄ and evaporated. The residue was purified by silica gel column chromatography with EtOAc (20%) in hexane to afford tert-butyl 3-(4-iodobutyl)azetidine-1-carboxylate (286 mg, 65%). (CD₃CN) δ (ppm) ¹H 3.94 (t, J=7.6 Hz, 2H), 3.47 (br, 2H), 3.27 (t, J=7.2 Hz, 2H), 2.52-2.48 (m, 1H), 1.81 (qt, J=7.2 Hz, 2H), 1.58 (q, J=7.6 Hz, 2H), 1.41 (s, 9H), 1.37-1.31 (m, 2H). LRMS (ESI): (calc) 339.2; (found) 362.0 (MNa)⁺.

To a stirred solution of tert-butyl 3-(4-iodobutyl)azetidine-1-carboxylate (286 mg, 0.84 mmol) in DMSO (4.5 mL) was added NaN₃ (110 mg, 1.69 mmol). The solution was stirred for 0.5 h and H₂O was added followed by EtOAc extractions. The organic phase was dried over Na₂SO₄ and evaporated. The residue was purified by silica gel column chromatography with EtOAc (20%) in hexane to afford 709 (189 mg, 88%).

(CDCl₃) δ (ppm) ¹H: 4.00 (t, J=8.4 Hz, 2H), 3.53 (dd, J=5.2, 8.4 Hz, 2H), 3.28 (t, J=6.8 Hz, 2H), 2.50-2.46 (m,1H), 1.60 (qt, J=7.2 Hz, 4H), 1.43 (s, 9H), 1.35-1.28 (m, 2H). LRMS (ESI): (calc) 254.3; (found) 277.2 (MNa)⁺.

### Step 5: tert-butyl 3-(4-(2-(4-fluorobenzyloxy)acetamido)butyl)azetidine-1-carboxylate (710)

Following the same procedure as described for compound 704, Example 500 but substituting 703 for azide 709, to afford 710 (120 mg, 41%). (CD₃CN) δ (ppm) ¹H: 7.42 (dd, J=5.6, 8.4 Hz, 2H), 7.14 (t, J=8.8 Hz, 2H), 6.86 (br, 1H), 4.55 (s, 2H), 3.92 (br, 2H), 3.91 (s, 2H), 3.47 (br, 2H), 3.19 (q, J=6.8 Hz, 2H), 2.49 (m, 1H), 1.58 (q, J=7.6 Hz, 2H), 1.46 (qt, J=7.2 Hz, 2H), 1.41 (s, 9H), 1.25 (m, 1H). LRMS (ESI): (calc) 394.5; (found) 417.3 (MNa)⁺.

### EXAMPLE 503a

### N-(2-(1H-indol-3-yl)ethyl)-3-(4-(2-(4-fluorobenzyloxy)acetamido)butyl)azetidine-1-carboxamide (711a)

To a stirred solution of tert-butyl 3-(4-(2-(4-fluorobenzyloxy)acetamido)butyl)azetidine-1-carboxylate 710 (110 mg, 0.28 mmol) in CH₂Cl₂ (1.5 mL) was added TFA (0.5 mL). After stirring for 1 h at room temperature, the solution was evaporated to dryness. To a stirred solution of 1,1′-carbonyldiimidazole (45 mg, 0.28 mmol) in THF (1.0 mL) was added dropwise a solution of tryptamine (45 mg, 0.28 mmol) in THF (1.0 mL). After stirring for 30 min at room temperature, a solution of the crude amine in THF (1.0 mL) was added and the resulting mixture was stirred overnight. A solution of 10% HCl was added and EtOAc extractions. The organic phase was dried over Na₂SO₄ and evaporated to dryness. The residue was purified by silica gel column chromatography with MeOH (5% to 10%) in EtOAc and by HPLC to afford 711a (40 mg, 30%).

(MEOD-d₄) δ (ppm) ¹H: 10.20 (br, 1H), 7.90 (br, 1H), 7.56 (d, J=8.0 Hz, 1H), 7.40 (dd, J=5.2, 8.4 Hz, 2H), 7.31 (d, J=8.0 Hz, 1H), 7.11-7.05 (m, 4H), 6.99 (t, J=7.2 Hz, 1H), 6.14 (br, 1H), 4.56 (s, 2H), 3.92 (s, 2H), 3.90 (t, J=8.0 Hz, 2H), 3.44 (t, J=5.6 Hz, 2H), 3.38 (t, J=7.2 Hz, 2H), 3.21 (q, J=6.4 Hz, 2H), 2.91 (t,. J=7.2 Hz, 2H), 2.53-2.46 (m, 1H), 1.59-1.47 (m, 4H), 1.28-1.21 (m, 2H). LRMS (ESI): (calc) 480.6; (found) 481.3 (MH)⁺.

### EXAMPLES 503b

### 3-(4-(2-(4-fluorobenzyloxy)acetamido)butyl)-N-((1-methyl-1H-benzo[d]imidazol-2-yl)methyl)azetidine-1-carboxamide (711b)

Compound 711b was prepared (36 mg, 35%) according to the method desribed for compound 711a, Example 503a, but replacing tryptamine with (1-methyl-1H-benzo[d]imidazol-2-yl)methanamine.

(MEOD-d₄) δ (ppm) ¹H: 8.16 (br, 1H), 7.91 (br, 1H), 7.62 (d, J=7.2 Hz, 1H), 7.54 (d, J=7.2 Hz, 1H), 7.41-7.38 (m, 2H), 7.37-7.29 (m, 2H), 7.08 (t, J=8.4 Hz, 2H), 4.64 (s, 2H), 4.56 (s, 2H), 4.05 (t, J=8.0 Hz, 2H), 3.92 (s, 2H), 3.87 (s, 3H), 3.58 (t, J=5.6 Hz, 2H), 3.23 (q, J=6.8 Hz, 2H), 2.57 (qt, J=6.0 Hz, 1H), 1.61 (q, J=8.0 Hz, 2H), 1.52 (qt, J=7.2 Hz, 2H), 1.31-1.24 (m, 2H). LRMS (ESI): (calc) 481.6; (found) 482.3 (MH)⁺.

### EXAMPLES 503c

### N-(4-(1-(2-(1H-indol-3-yl)ethylcarbamothioyl)azetidin-3-yl)butyl)-2-(4-fluorobenzyloxy)acetamide (711c)

Compound 711b was prepared (26 mg, 22%) according to the method desribed for compound 711a, Example 503a, but replacing 1,1′-carbonyldiimidazole with 1,1′-thiocarbonyldiimidazole. (MEOD-d₄) δ (ppm) ¹H, 9.18 (br, 1H), 7.67 (d, J=7.6 Hz, 1H), 7.44-7.40 (m, 3H), 7.16-7.12 (m, 3H), 7.07 (t, J=7.6 Hz, 1H), 6.89 (br, 1H), 6.00 (br, 1H), 4.55 (s, 2H), 4.01 (br, 2H), 3.92 (s, 2H), 3.75 (q, J=6.4 Hz, 2H), 3.55 (br, 2H), 3.20 (q, J=6.4 Hz, 2H), 3.00 (t, J=7.2 Hz, 2H), 2.52-2.48 (m, 1H), 2.25 (br, 1H), 1.56 (q, J=7.6 Hz, 2H), 1.48 (qt, J=7.2 Hz, 2H), 1.29-1.21 (m, 2H). LRMS (ESI): (calc) 496.6; (found) 497.2 (MH)⁺.

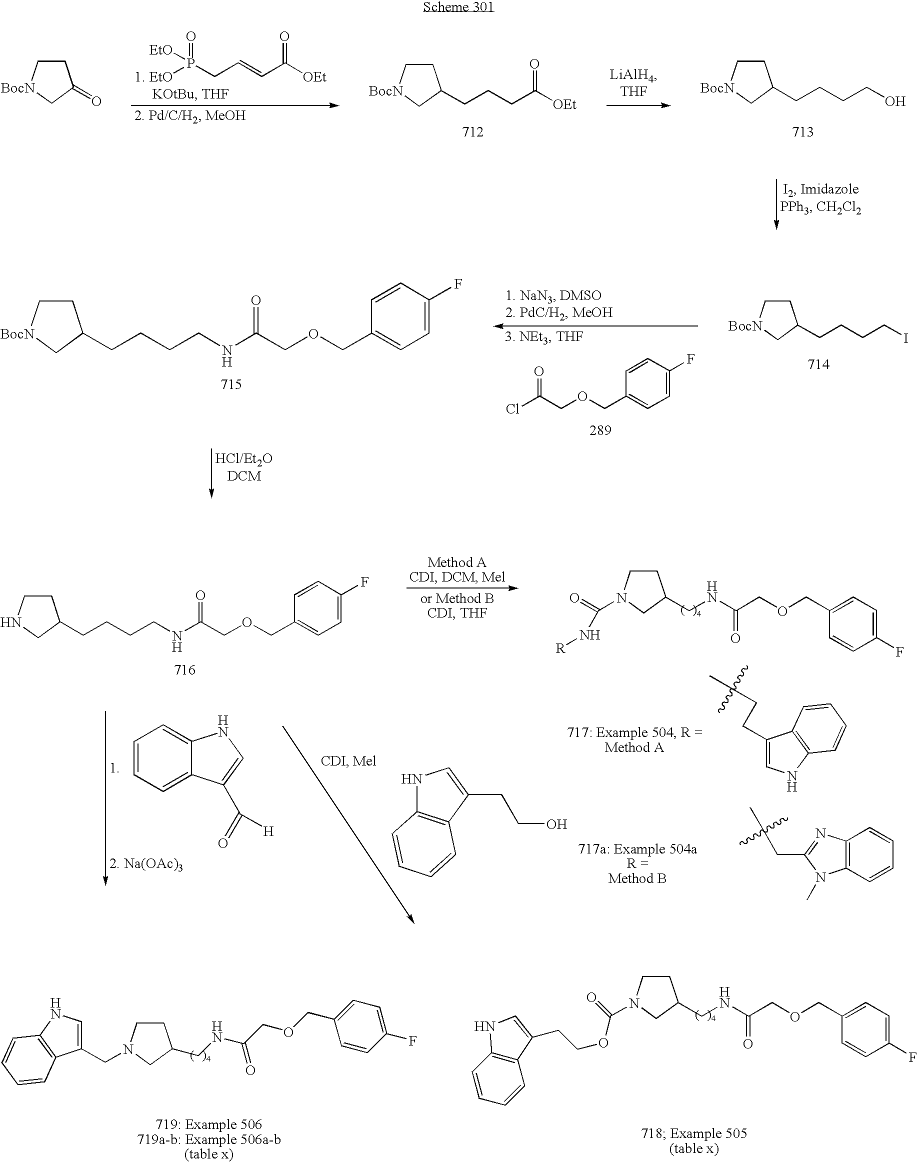

### EXAMPLE 504

### N-(2-(1H-indol-3-yl)ethyl)-3-(4-(2-(4-fluorobenzyloxy)acetamido)butyl)pyrrolidine-1-carboxamide (717)

### Step 1: tert-butyl 3-(4-ethoxy-4-oxobutyl)pyrrolidine-1-carboxylate (712)

Triethyl 4-phosphonocrotonate (1.44 g, 6.47 mmol) was added to a mixture of potassium tert-butoxide (726 mg, 6.47 mmol) in THF (10 mL) at 0° C. After 10 min, 1-N-Boc-3-pyrrolidinone (1.00 g, 5.39 mmol) was added to the mixture and the reaction was warmed up slowly to room temperature, and stirred for 1.5 h. A solution of sodium carbonate was added and the aqueous mixture was extracted with EtOAc and the organic extract was dried (Na₂SO₄), filtered, and evaporated. To give (E)-tert-butyl 3-((E)-4-ethoxy-4-oxobut-2-enylidene)pyrrolidine-1-carboxylate which was used without further purification. LRMS (ESI): (calc) 281.3; (found) 304.3 (MNa)⁺.

The unsaturated ester from above (1.52 g, 5.39 mmol) and 10% Palladium on carbon (20% wt, 300 mg) in MeOH (27 mL) and EtOAc (1 mL) was hydrogenated under 1 atmosphere of H₂ for 2 h. The material was purified by silica gel column chromatography with ethyl acetate (10%) in hexane to give 712 (610 mg 40% over 2 steps).

(MEOD-d₄) δ (ppm) ¹H, 4.11 (q, J=7.2 Hz, 2H), 3.53-3:47 (m, 1H), 3.44-3.39 (m, 1H), 3.27-3.18 (m, 1H), 3.87-3.81 (m, 1H), 2.33 (t, J=7.4 Hz, 2H), 2.16-2.09 (m, 2H), 2.05-2.00 (m, 1H), 1.68-1.59 (m, 2H), 1.58-1.48 (m, 12H), 1.24 (t, J=7.2 Hz, 3H).

LRMS (ESI): (calc) 285.3; (found).308.2 (MNa)+

### Step 2: tert-butyl 3-(4-hydroxybutyl)pyrrolidine-1-carboxylate (713)

A solution of ester 712 (639 mg, 2.23 mmol) in THF (3 mL) was added to a mixture of lithium aluminumhydride (254 mg, 6.71 mmol) in THF (3 mL) at room temperature. The reaction stirred for 30 min. The reaction was cooled to 0° C. and slowly quenched with H₂O until the grey mixture turned white. The reaction was diluted with 10% HCl in water. The aqueous mixture was extracted with ethyl acetate. The organic extract was dried (Na₂SO₄), filtered, and evaporated. Product 713 was used without further purification (522 mg, 96%). (MEOD-d₄) δ (ppm) ¹H, 3.55 (t, J=6.4 Hz, 2H), 3.52-3.47 (m, 1H), 3.44-3.39 (m, 1H), 3.29-3.18 (m, 1H), 2.86-2.81 (m, 1H), 2.14-2.13 (m, 1H), 2.04-1.98 (m, 2H), 1.60-1.48 (m, 3H), 1.45 (s, 9H), 1.40-1.35 (m, 4H).

LRMS (ESI): (calc) 243.3; (found) 266.2 (MNa)⁺.

### Step 3: tert-butyl 3-(4-iodobutyl)pyrrolidine-1-carboxylate (714)

Imidazole (160 mg, 2.36 mmol), iodine (598 mg, 2.36 mmol) and 713 (522 mg, 2.15 mmol) were added in the mentioned order to a solution of triphenylphosphine (618 mg, 2.36 mmol) in DCM (10 mL). The bright orange mixture stirred for 16 h. The formed solid was filtered off and the filtrate was evaporated. The residue was purified by silica gel column chromatography with EtOAc (20%) in Hexane to give 714 (574 mg 69%).

(MEOD-d₄) δ (ppm) ¹H: 3.52-3.48 (m, 1H), 3.44-3.40 (m, 1H), 3.26-3.18 (m, 3H), 3.87-3.81 (m, 1H), 2.16-2.10 (m, 1H), 2.05-2.01 (m, 1H), 1.85-1.78 (m, 2H), 1.60-1.45 (m, 14H).

LRMS (ESI): (calc) 353.2; (found).376.2 (MNa)+

### Step 4: tert-butyl 3-(4-(2-(4-fluorobenzyloxy)acetamido)butyl)pyrrolidine-1-carboxylate (715)

A solution of sodium azide (211 mg, 3.24 mmol) and 714 (574 mg, 1.62 mmol) in DMSO (8 mL) stirred at room temperature for 6 h. The reaction was diluted with H₂O. The aqueous mixture was extracted with ethyl acetate. The organic extract was dried (Na₂SO₄), filtered, and evaporated to give the crude azide, tert-butyl 3-(4-azidobutyl)pyrrolidine-1-carboxylate. LRMS (ESI): (calc) 268.3; (found) 291.2 (MNa)⁺.

The crude azide (435 g, 1.62 mmol) in methanol (8 mL) and 10% Palladium on carbon (20% wt, 87 mg) in ethyl acetate (1 mL) was hydrogenated under 1 atmosphere of H₂ for 16 h, to give the crude amine, tert-butyl 3-(4-aminobutyl)pyrrolidine-1-carboxylate. LRMS (ESI): (calc) 242.3; (found) 243.3 (MH)⁺.

A solution of acid chloride 289 (1.67 mmol) in THF (5 mL) was added to the crude amine from above (445 mg, 1.84 mmol) and triethylamine (0.436 mL, 3.35 mmol) in THF (5 mL). The reaction was stirred at room temperature for 3 h, then was diluted with 10% HCl in water and extracted with ethyl acetate. The organic extract was dried (Na₂SO₄), filtered, and evaporated and the residue was purified by silica gel column chromatography with ethyl acetate (30%-60%) in Hexanes to give 715 (196 mg 28% over 3 steps).

(CDCl₃) δ (ppm) ¹H: 7.32-7.30 (m, 2H), 7.08-7.04 (m, 2H), 6.53 (bs, 1H), 4.53 (s, 2H), 3.96 (s, 2H), 3.56-3.37 (m, 2H), 3.29-3.20 (m, 3H), 2.88-2.77 (m, 1H), 2.07-2.00 (m, 1H), 1.99-1.91 (m, 1H), 1.56-1.48 (m, 3H), 1.45 (s, 9H), 1.43-1.29 (m, 4H).

LRMS (ESI): (calc) 408.5; (found) 431.2 (MNa)⁺.

### Step 5: 2-(4-fluorobenzyloxy)-N-(4-(pyrrolidin-3-yl)butyl)acetamide (716)

A solution of dry HCl (4M, 1.19 mL, 4.79 mmol) in dioxane was added to 715 (196 mg, 0.479 mmol) in minimum amount of DCM (1 mL). The reaction stirred at room temperature for 2 h. The solvent was evaporated and the residue was triturated with ether to give the HCl salt of amine 716 (160 mg, 97%).

(MEOD-d₄) δ (ppm) ¹H: 7.43-7.39 (m, 2H), 7.11-7.06 (m, 2H), 4.57 (s, 2H), 3.94 (s, 2H), 3.86-3.33 (m, 2H), 3.26-3.17 (m, 3H), 2.81-2.76 (m, 1H), 2.31-2.13 (m, 2H), 1.64-1.44 (m, 7H). LRMS (ESI): (calc) 308.3; (found) 309.2 (MNa)⁺.

### Step 6: N-(2-(1H-indol-3-yl)ethyl)-3-(4-(2-(4-fluorobenzyloxy)acetamido)-butyl)pyrrolidine-1-carboxamide (717)

Method A: Tryptamine (104 mg, 0.648 mmol) and CDI (420 mg, 2.59 mmol) in DCM (2 mL) were stirred at room temperature for 2 h. The reaction was diluted with H₂O, and the aqueous mixture was extracted with EtOAc and the organic extract was dried (Na₂SO₄), filtered, and evaporated. The residue was dissolved in acetonitrile (0.5 mL) and a solution of methyliodide (0.045 mL, 0.486 mmol) was added and the reaction was stirred for 2 h, concentrated and a solution of amine 716 (100 mg, 0.324 mmol) in acetonitrile (0.5 mL) was added. The reaction was stirred for 16 h, and then it was diluted with 10% NaOH in water and the aqueous mixture was extracted with ethyl acetate and the organic extract was dried (Na₂SO₄), filtered, and evaporated. The residue was purified by silica gel column chromatography with methanol (1%-2%) in DCM to give 717 (65 mg, 40%).

(MEOD-d₄) δ (ppm) ¹H, 7.55 (d, J=8 Hz, 1H), 7.42-7.38 (m, 2H), 7.31 (d, J=8 Hz, 1H), 7.10-7.05 (m, 4H), 6.99-6.95 (m, 1H), 4.56 (s, 2H), 3.93 (s, 2H), 3.44-3.34 (m, 4H), 3.24-3.17 (m, 3H), 2.95-2.91(m, 2H), 2.80-2.76 (m, 1H), 2.14-2.10 (m, 1H), 2.03-1.99 (m, 1H), 1.55-1.44 (m, 3H), 1.40-1.30 (m, 4H).

LRMS (ESI): (calc) 494.6; (found) 495.3 (MH)⁺.

### EXAMPLE 504a

### 3-(4-(2-(4-fluorobenzyloxy)acetamido)butyl)-N-((1-methyl-1H-benzo[d]imidazol-2-yl)methyl)pyrrolidine-1-carboxamide (717a)

Method B: A solution of (1-methyl-1H-benzo[d]imidazol-2-yl)methanamine (39 mg, 0.243 mmol) in THF (0.5 mL) was added to CDI (39 mg, 0.243 mmol) in THF (1 mL). The reaction was stirred for 30 min then a solution of 716 (75 mg, 0.243 mmol) in DMF (0.5 mL) was added and the mixture was stirred for 16 h. The reaction was diluted with H₂O and the aqueous layer was extracted with ethyl acetate. The organic extract was dried (Na₂SO₄), filtered, and evaporated. The residue was purified by silica gel column chromatography with methanol (1%-3%) in dichloromethane to give 717a (45 mg, 37%).

(MEOD-d₄) δ (ppm) ¹H: 7.59 (d, J=7.6 Hz, 1H), 7.47 (d, J=8.0 Hz, 1H), 7.41-7.38 (m, 2H), 7.29-7.21 (m, 2H), 7.10-7.05 (m, 2H), 4.63 (s, 2H), 4.55 (s, 2H), 3.92 (s, 2H), 3.84 (s, 3H), 3.61-3.52 (m, 1H), 3.51-3.45 (m, 1H), 3.34-3.29 (m, 1H), 3.23 (t, J=7.0 Hz, 2H), 2.92 (t, J=9.2 Hz, 1H), 2.19-2.06 (m, 2H), 1.55-1.47 (m, 3H), 1.45-1.33 (m, 4H).

LRMS (ESI): (calc) 495.5; (found) 496.3 (MH)⁺.

### EXAMPLE 505

### 2-(1H-indol-3-yl)ethyl 3-(4-(2-(4-fluorobenzyloxy)acetamido)butyl)pyrrolidine-1-carboxylate (718)

Compound 718, Example 505, was prepared using method A of Example 504 replacing tryptamine with 2-(1H-indol-3-yl)ethanol.

(MEOD-d₄) δ (ppm) ¹H: 7.53 (d, J=8.4 Hz, 1H), 7.39-7.36 (m, 2H), 7.31 (d, J=8.0 Hz, 1H), 7.08-7.03 (m, 4H), 6.98-6.96 (m, 1H), 4.56 (s, 2H), 4.27 (t, J=6.8 Hz, 2H), 3.91 (s, 2H), 3.51-3.29 (m, 2H), 3.24-3.13 (m, 3H), 3.05 (t, J=6.8 Hz, 2H), 2.85-2.70 (m, 1H), 2.05-1.90 (m, 2H), 1.52-1.22 (m, 7H).

LRMS: (calc) 495.6; (found) 496.3 (MH)⁺.

### EXAMPLE 506

### N-(4-(1-((1H-indol-3-yl)methyl)pyrrolidin-3-yl)butyl)-2-(4-fluorobenzyloxy)-acetamide (719)

A solution of 1H-indole-3-carbaldehyde (38 mg, 0.243 mmol) and 716 (75 mg, 0.243 mmol) in THF (2 ml) was stirred at room temperature for 1 h. Sodium triacetoxyborohydride (51 mg, 0.243 mmol) was added and the mixture stirred for 16 h. The reaction was diluted with 10% NaOH in water. The aqueous mixture was extracted with ethyl acetate. The organic extract was dried (Na₂SO₄), filtered, and evaporated. The residue was purified by prep-hplc with gradient of methanol (20%-95%) in water to give 719 (50 mg, 47%).

(MEOD-d₄) δ (ppm) ¹H: 8.50 (s, 1H), 7.71 (d, J=8.0 Hz, 1H), 7.51 (s, 1H), 7.43 (d, J=8.0 Hz, 1H), 7.39-7.35 (m, 2H), 7.20-7.11 (m, 2H), 7.08-7.04 (m, 2H), 4.53 (s, 2H), 4.50 (s, 2H), 3.90 (s, 2H), 3.51-3.46 (m, 1H), 3.36-3.31 (m, 2H), 3.19 (t, J=7.0 Hz, 2H), 2.93-2.88 (m, 1H), 2.34-2.26 (m, 1H), 2.19-2.11 (m, 1H), 1.66-1.57 (m, 1H), 1.51-1.38 (m, 4H), 1.36-1.26 (m, 2H). LRMS: 437.5 (calc) 438.3 (found, M+1)

Examples 506a-b describe the preparation of compounds 719a-b using the same procedure as described for compound 719, Example 506, in scheme 301, replacing 1H-indole-3-carbaldehyde with 6-methoxy-1H-indole-3-carbaldehyde and 6-fluoro-1H-indole-3-carbaldehyde respectively. Characterization data are presented in Table 30.

TABLE 30

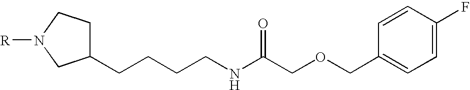

| Ex | Cpd | R₃ | Name | Characterization | Scheme |
|---|---|---|---|---|---|
| 506a | 719a | 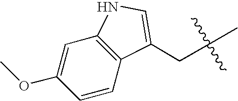 | 2-(4- fluorobenzyloxy)- N-(4-(1-((5- methoxy-1H- indol-2- yl)methyl) pyrrolidin- 3-yl)butyl) acetamide | ¹H NMR: (MEOD-d₄) δ (ppm): 8.52 (s, 1H), 7.46 (s, 1H), 7.40-7.36 (m, 2H), 7.31 (d, J=8.8Hz, 1H), 7.22 (d, J=2.4Hz, 1H), 7.09-7.05 (m, 2H), 6.84 (dd, J=8.8, 2.4Hz, 1H), 4.54 (s, 2H), 4.50 (s, 2H), 3.90 (s, 2H), 3.84 (s, 3H), 3.53-3.49 (m, 1H), | |
| # 3.38-3.34 (m, 2H), 3.20 (t, J=7.2Hz, 2H), 2.95-2.90 (m, 1H), 2.37-2.29 (m, 1H), 2.22-2.14 (m, 1H), 1.70-1.60 (m, 1H), 1.53-1.37 (m, 4H), 1.35-1.22 (m, 2H). | 301 example 506 | | | | |
| 506b | 719b | 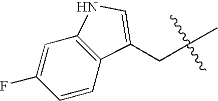 | N-(4-(1-((5- fluoro-1H-indol- 2-yl)methyl) pyrrolidin-3- yl)butyl)-2-(4- fluorobenzyloxy) acetamide | ¹H NMR: (MEOD-d₄) δ (ppm): 8.49 (s, 1H), 7.58 (s, 1H), 7.47 (dd, J=10, 2.4Hz, 1H), 7.42-7.36 (m, 3H), 7.09-7.04 (m, 2H), 6.96 (td, J=6.9, 1.8Hz, 1H), 4.54 (s, 2H), 4.50 (s, 2H), 3.91 (s, 2H), 3.52-3.47 (m, 1H), 3.36-3.34 (m, 2H), 3.20 (t, J=7.0Hz, 2H), | |
| # 2.96-2.88 (m, 1H), 3.39-3.32 (m, 1H), 2.20-2.15 (m, 1H), 1.67-1.62 (m, 1H), 1.53-1.39 (m, 4H), 1.35-1.23 (m, 2H). | 301 example 506 | | | | |

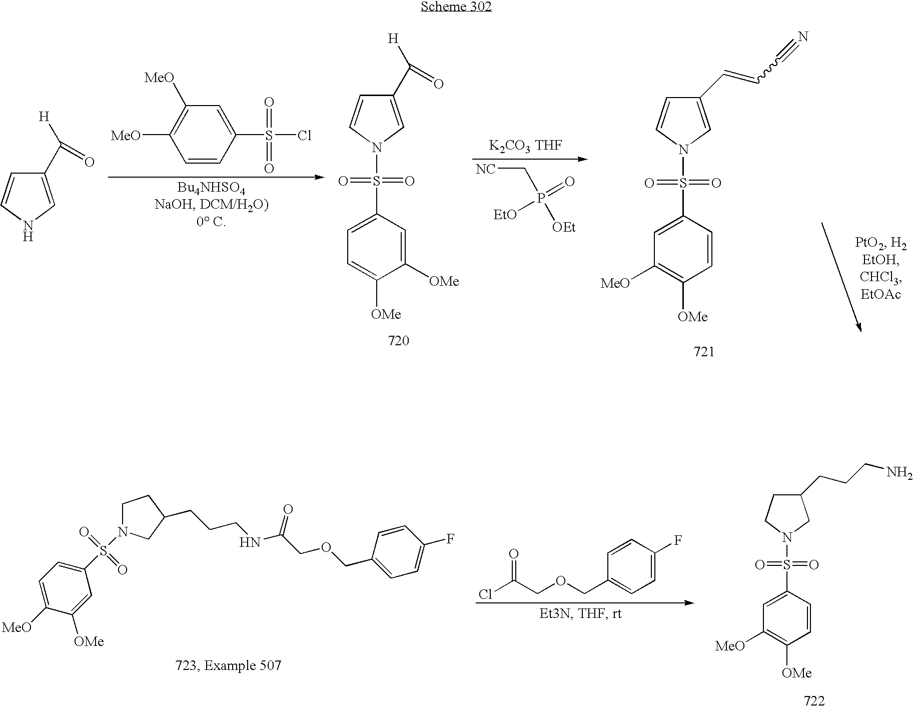

### EXAMPLE 507

### N-(3-(1-(3,4-dimethoxyphenylsulfonyl)pyrrolidin-3-yl)propyl)-2-(4-fluorobenzyloxy)acetamide (723)

### Step 1: 1-(3,4-dimethoxyphenylsulfonyl)-1H-pyrrole-3-carbaldehyde (720)

3,4-dimethoxybenzene-1-sulfonyl chloride (755 mg, 3.2 mmol, 1.1 eq.) in DCM (3.8 ml) was added to an ice cold mixture of 50% NaOH (3 ml), Bu₄NHSO₄ (17 mg), DCM (3.4 ml) and 1H-pyrrole-3-carbaldehyde (276 mg, 2.9 mmol, prepared according to the method of Brian L. Bray et al, JOC, 1990, 55, 6317-6328; Brian L. Bray et al, JOC, 1988, 53, 6115-8). The reaction was allowed to warm-up to room temperature over 2 h and was stirred at room temperature for 16 h. Saturated NaHCO₃ was added and the organic layer was separated and the aqueous layer extracted with DCM (×2). The organic extracts were washed with sat'd NaHCO₃, dried (MgSO₄), filtered and concentrated leaving a beige solid which was triturated from ether to give 720 (85% yield).

(CDCl₃) δ (ppm) ¹H: 9.81 (s, 1H0, 7.76 (m, 1H), 7.58 (dxd, J=2.3, 8.6 Hz, 1H), 7.31 (d, J=2.3 Hz, 1H), 7.17 (m, 1H), 6.95 (d, J=8.6 Hz, 1H), 6.7 (m, 1H), 3.94 (s, 3H), 3.93 (s, 3H). LRMS (ESI): (calc.)295.3; (found) 295.9 (MH)⁺

### Step 2: 3-(1-(3,4-dimethoxyphenylsulfonyl)-1H-pyrrol-3-yl)acrylonitrile (721)

A mixture of diethyl cyanomethylphosphonate (330 mg/302 uL, 1.83 mmol, 1.2 eq.) and K₂CO₃ (211 mg, 1.53 mmol) in THF (6 ml) was stirred at room temperature for 10 min, then at 70° C. for 20 min after which aldehyde 720 (450 mg, 1.52 mmol) was added and the mixture was stirred at 70° C. for 16 h. The material was taken to dryness and the residue was purified by silica gel column chromatography with EtOAc (30-40%) in Hexane to afford 721 (298 mg, 61.6% yield, as a mixture of 4:1 trans:cis isomers)

LRMS (ESI): (calc.) 318.4; (found) 319.0 (MH)⁺

### Step 3: 3-(1-(3,4-dimethoxyphenylsulfonyl)pyrrolidin-3-yl)propan-1-amine (722)

Nitrile 721 (298 mg, 0.94 mmol), platinum oxide (36 mg) in EtOH (8 ml), CHCl₃ (0.6 ml), and EtOAc (1.6 ml) (to help solublise the compound) were placed under H₂ atmosphere. The material was not fully soluble. After 16 h a sample checked by MS indicated the presence of the ring reduced amine 722. The catalyst was filtered through Celite, and the filtrate was concentrated and triturated from ether, leaving 722 as white solid (119 mg, 38.5% yield, ˜85% pure by HPLC).

LRMS (ESI): (calc.) 328.4; (found) 329.0 (MH)⁺

### Step 4: N-(3-(1-(3,4-dimethoxyphenylsulfonyl)pyrrolidin-3-yl)propyl)-2-(4-fluorobenzyloxy)acetamide (723)

Amine 722 (100 mg, 0.30 mmol) was added to acid chloride 289 (0.4 mmol) in THF (3 ml) followed by TEA (0.101 g/139.4 uL, 1 mmol). After 16 h at room temperature, EtOAc and sat'd NaHCO₃ were added and the organic layer was separated, washed with H₂O, 0.5 N HCl, brine, dried (MgSO₄), filtered and concentrated leaving a white solid. Trituration from ether, then from EtOAc/hexanes gave 723 as white solid (77 mg, 52% yield). (CD₃CN) δ (ppm) ¹H: 7.41 (m,m, 2H), 7.27 (d, J=2.2 Hz, 1H), 7.11 (m, 3H), 6.83 (bs, 1H), 4.53 (s, 2H), 3.89 (s, 5H), 3.88 (s, 3H), 3.4 (dxd, J=7.5, 9.9 Hz, 1H), 3.3 (m, 1H), 3.13 (m, 3H), 2.75 (dxd, J=7.9, 9.9 Hz, 1H), 1.93 (m, 1H), 1.38 (m, 3H), 1.2 (m, 2H).

LRMS (ESI): (calc.) 494.6; (found) 495.0 (MH)⁺

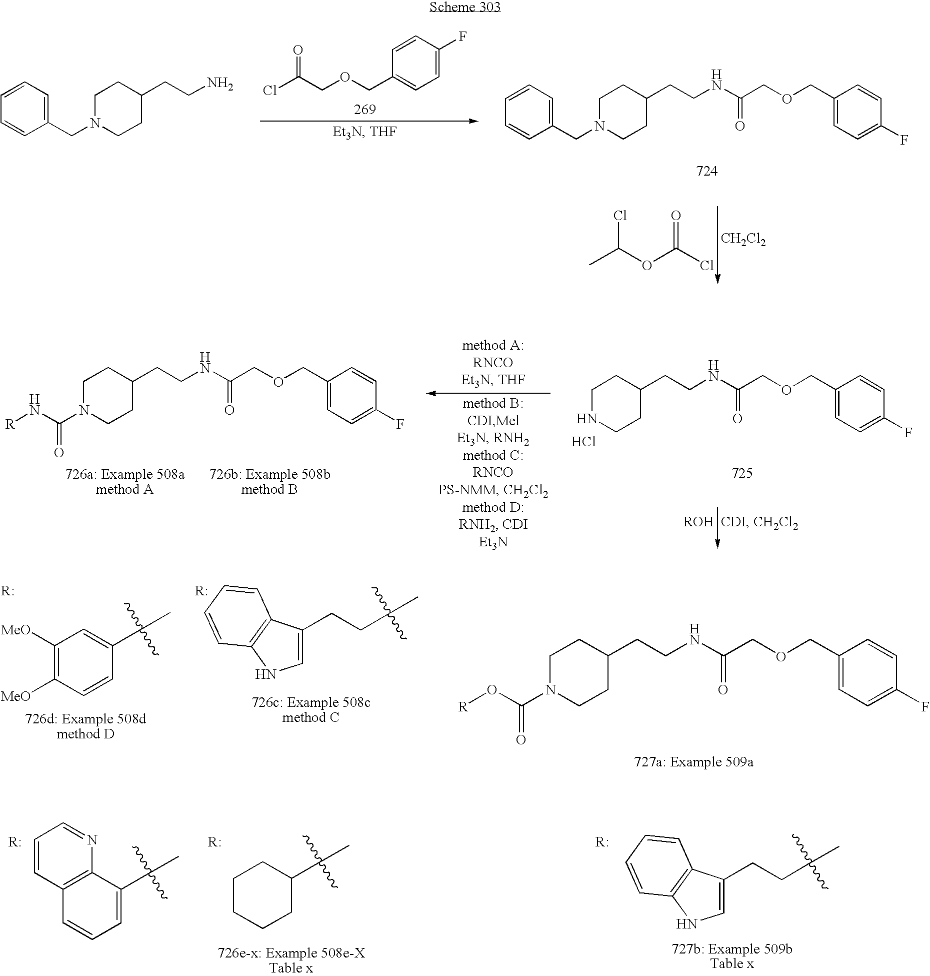

### EXAMPLE 508a

### 4-(2-(2-(4-fluorobenzyloxy)acetamido)ethyl)-N-(3,4-dimethoxyphenyl)piperidine-1 carboxamide (726a)

### Step 1: 2-(4-fluorobenzyloxy)-N-(2-(1-benzylpiperidin-4-yl)ethyl)acetamide (724)

To a stirred solution of acid chloride 289 (16.5 mmol) and triethylamine (4.6 mL, 33 mmol) in THF (60 mL) at 0° C. was added a solution of 4-(2-aminoethyl)-1-benzylpiperidine (4.0 mL, 18.1 mmol) in THF (6 mL) dropwise. The reaction was stirred at room temperature for 16 h. The reaction mixture was quenched with a saturated solution of sodium carbonate in water. The aqueous mixture was extracted with ethyl acetate. The organic extract was dried (MgSO₄), filtered, and evaporated. The residue was purified by silica gel column chromatography with gradient of methanol (0-10%) in dichloromethane to give 724 (4.87 g, 70%).

(DMSO-d₆) δ (ppm) ¹H: 7.75 (t, J=5.9 Hz, 1H), 7.40 (dd, J=8.4 and 5.7 Hz, 2H), 7.30-7.09 (m, 7H), 4.48 (s, 2H), 3.84 (s, 2H), 3.39 (s, 2H), 3.10 (q, J=7.2 Hz, 2H), 2.73 (bd, J=12 Hz, 2H), 1.83 (bt, J=9.8 Hz, 2H), 1.59 (bd, J=12 Hz, 2H), 1.33 (q, J=7.4 Hz, 2H), 1.28-1.13 (m, 1H), 1.08 (qd, J=12 and 3.5 Hz, 2H). LRMS (ESI): (calc) 384.2; (found) 385.2 (MH)⁺.

### Step 2: 2-(4-fluorobenzyloxy)-N-(2-(piperidin-4-yl)ethyl)acetamide hydrochloride (725)

To a stirred solution of 724 (4.87 g, 12.7 mmol) in dichloromethane (64 mL) was added 1-chloroethyl chloroformate (2.74 mL, 25.4 mmol). The reaction was stirred at room temperature for 1.5 h. All the solvent was evaporated then the residue was stirred in methanol (40 mL) at 50° C. for 45 min. The solvent was evaporated then the residue was purified by silica gel column chromatography with gradient of methanol (0-20%) in dichloromethane to give 725 (3.10 g, 74%).

(DMSO-d₆) δ (ppm) ¹H, 8.97-8.87 (m, 1H), 8.76-8.63 (m, 1H), 7.84 (t, J=5.9 Hz, 1H), 7.42 (dd, J=8.4 and 5.7 Hz, 2H), 7.18 (t, J=8.8 Hz, 2H), 4.50 (s, 2H), 3.86 (s, 2H), 3.19 (bd, J=13 Hz, 2H), 3.12 (q, J=6.8 Hz, 2H), 2.76 (bq, J=11 Hz, 2H), 1.79 (bd, J=13 Hz, 2H), 1.58-1.42 (m, 1H), 1.36 (q, J=7.0 Hz, 2H), 1.30-1.25 (m, 2H). LRMS (ESI): (calc) 330.2; (found) 295.2 (M−Cl)⁺.

### Step 3: 4-(2-(2-(4-fluorobenzyloxy)acetamido)ethyl)-N-(3,4-dimethoxyphenyl)piperidine-1-carboxamide (726a)

Method A:

To a stirred solution of 725 (40 mg, 0.12 mmol) in THF (1 mL) was added triethylamine (0.10 mL, 0.69 mmol), then 3,4-dimethoxyphenylisocyanate (18 uL, 0.12 mmol). The reaction was stirred at room temperature for 16 h. More 3,4-dimethoxyphenylisocyanate (18 uL, 0.12 mmol) was added. The reaction was stirred at room temperature for 1 h. The reaction mixture was quenched with water. The aqueous mixture was extracted with dichloromethane. The organic extract was dried (MgSO₄), filtered, and evaporated. The residue was purified by prep-hplc with gradient of methanol (20-100% in water to give 726a (27 mg, 49%).

(DMSO-d₆) δ (ppm) ¹H: 8.24 (s,1H), 7.81 (t, J=5.9 Hz, 1H), 7.42 (d, J=5.7 Hz, 1H), 7.40 (d, J=5.7 Hz, 1H), 7.17 (t, J=9.0 Hz, 2H), 7.13 (d, J=2.3 Hz, 1H), 6.94 (dd, J=8.8 and 2.5 Hz, 1H), 6.78 (d, J=8.8 Hz, 1H), 4.49 (s, 2H), 4.05 (bd, J=13 Hz, 2H), 3.85 (s, 2H), 3.67 (s, 3H), 3.66 (s, 3H), 3.13 (q, J=6.7 Hz, 2H), 2.67 (bt, J=12 Hz, 2H), 1.66 (bd, J=11 Hz, 2H), 1.40-1.33 (m, 3H), 1.04-0.96 (m, 2H). LRMS (ESI): (calc) 473.2; (found) 474.1 (MH)⁺.

### EXAMPLE 508b

### N-(2-(1H-indol-3-yl)ethyl)-4-(2-(2-(4-fluorobenzyloxy)acetamido)ethyl)piperidine-1-carboxamide (726b)

Method B:

To a stirred solution of 725 (53 mg, 0.18 mmol) in THF (3.6 mL) was added 1,1′-carbonyldiimidazole (117 mg, 0.72 mmol). The mixture was stirred for 4 h at 60° C. The solvent was evaporated, and the residue diluted with dichloromethane and washed with brine. The organic layer was dried (MgSO₄), filtered, and evaporated. Acetonitrile (1.0 mL) and iodomethane (0.25 mL) were added to the residue. The reaction was stirred at room temperature for 16 h. The solvent was evaporated and the residue was put in dichloromethane (1.0 mL), then tryptamine (87 mg, 0.54 mmol) was added, followed by triethylamine (0.25 mL). The reaction was stirred for 16 h at room temperature. The solvent was evaporated and the residue was purified by prep-hplc with gradient of methanol (20-100%) in water to give 726b (32 mg, 37%).

(DMSO-d₆) δ (ppm) ¹H: 10.76 (s, 1H), 7.78 (t, J=5.9 Hz, 1H), 7.52 (d, J=7.8 Hz, 1H), 7.41 (dd, J=8.8 and 5.7 Hz, 2H), 7.30 (d, J=8.0 Hz, 1H), 7.17 (t, J=9.0 Hz, 2H), 7.09 (d, J=2.3 Hz, 1H), 7.03 (td, J=7.0 and 1.2 Hz, 1H), 6.94 (t, J=6.8 Hz, 1H), 6.52 (t, J=5.3 Hz, 1H), 4.49 (s, 2H), 3.91 (bd, J=13 Hz, 2H), 3.85 (s, 2H), 3.27-3.22 (m, 2H), 3.12 (q, J=6.3 Hz, 2H), 2.78 (t, J=8.4 Hz, 2H), 2.56 (t, J=12 Hz, 2H), 1.59 (bd, J=12 Hz, 2H), 1.35-1.30 (m, 3H), 0.96-0.88 (m, 2H). LRMS (ESI): (calc) 480.3; (found) 481.2 (MH)⁺.

### EXAMPLE 508c

### N-cyclohexyl-4-(2-(2-(4-fluorobenzyloxy)acetamido)ethyl)piperidine-1-carboxamide (726c)

Method C:

To a stirred solution of PS-NMM (300 mg, 0.68 mmol) in dichloromethane (1.0 mL) was added 725 (83 mg, 0.25 mmol) in dichloromethane (1.5 mL), followed by cyclohexyl isocyanate (29 uL, 0.23 mmol). The reaction was stirred at room temperature for 16 h. PS-NMM was filtered, solvent evaporated and the residue was purified by prep-hplc with gradient of methanol (20-100%) in water to give 726c (54 mg, 56%).

(DMSO-d₆) δ (ppm) ¹H, 7.78 (t, J=5.9 Hz, 1H), 7.42 (dd, J=8.4 and 5.7 Hz, 2H), 7.18 (t, J=8.8 Hz, 2H), 6.03 (d, J=7.6 Hz, 1H), 4.49 (s, 2H), 3.91 (bd, J=13 Hz, 2H), 3.85 (s, 2H), 3.40-3.33 (m, 1H), 3.12 (q, J=6.3 Hz, 2H), 2.55-2.50 (m, 2H), 1.71-1.64 (m, 4H), 1.60-1.53 (m, 3H), 1.40-1.28 (m, 3H), 1.28-1.00 (m, 5H), 1.00-0.88 (m, 2H). LRMS (ESI): (calc) 419.3; (found) 420.3 (MH)⁺.

### EXAMPLE 508d

### 4-(2-(2-(4-fluorobenzyloxy)acetamido)ethyl)-N-(quinolin-8-yl)piperidine-1-carboxamide (726d)

Method D:

To a stirred solution of 8-aminoquinoline (36 mg, 0.25 mmol) in THF (1.0 mL) was added 1,1′-carbonyldiimidazole (40 mg, 0.25 mmol). The mixture was stirred for 1 h at 60° C. To that intermediate was added 725 (90 mg, 0.30 mmol), triethylamine (0.09 mL, 0.63 mmol) and DCM (0.5 mL). The reaction was stirred at room temperature for 16 h. The solvent was evaporated and the residue was purified by prep-hplc with gradient of methanol (20-100%) in water to give 726d (50 mg, 43%).

(DMSO-d₆) δ (ppm) ¹H: 9.32 (s, 1H), 8.88 (dd, J=4.3 and 1.6 Hz, 1H), 8.41-8.34 (m, 2H), 7.82 (t, J=5.9 Hz, 1H), 7.61 (dd, J=8.2 and 4.3 Hz, 1H), 7.52 (d, J=4.5 Hz, 2H), 7.43 (dd, J=8.4 and 5.7 Hz, 2H), 7.18 (t, J=8.8 Hz, 2H), 4.51 (s, 2H), 4.09 (bd, J=13 Hz, 2H), 3.87 (s, 2H), 3.17 (q, J=6.7 Hz, 2H), 1.78 (bd, J=11 Hz, 2H), 1.58-1.45 (m, 1H), 1.40 (q, J=7.0 Hz, 2H), 1.11 (qd, J=12 and 3.5 Hz, 2H). LRMS (ESI): (calc) 464.2; (found)465.3 (MH)⁺.

Example 508e-p describe the preparation of compound 726e-p using the same procedures as described in scheme 303, utilizing method A, B, C or D. Characterization data are presented in Table 31.

TABLE 31

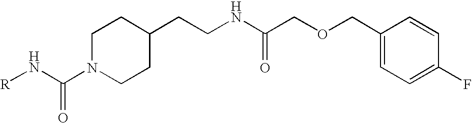

| Ex | Cpd | R | Name | Characterization | Scheme |
|---|---|---|---|---|---|
| 508e | 726e | 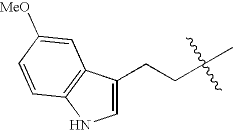 | 4-(2-(2-(4- fluorobenzyloxy) acetamido)ethyl)- N-(2-(5-methoxy- 1H-indol-3- yl)ethyl) piperidine- 1-carboxamide | (DMSO-d₆) δ (ppm) ¹H: 10.60 (s, 1H), 7.79 (t, J=5.9Hz, 1H), 7.43 (d, J=6.1Hz, 1H), 7.41 (d, J=5.7Hz, 1H), 7.20 (d, J=8.6Hz, 1H), 7.18 (t, J=9.2Hz, 2H), 7.04 (dd, J=18 and 2.3Hz, 2H), 6.69 (dd, J=8.6 and 2.3 Hz, 1H), 6.51 (t, J=5.3Hz, 1H), 4.50 | |
| # (s, 2H), 3.92 (bd, J=13Hz, 2H), 3.86 (s, 2H), 3.74 (s, 3H), 3.25 (q, J=8.2Hz, 2H), 3.13 (q, J=6.3Hz, 2H), 2.75 (t, J=7.8Hz, 2H), 2.56 (t, J=12Hz, 2H), 1.60 (d, J=12Hz, 2H), 1.41-1.30 (m, 3H), 0.93 (q, J=7.6Hz, 2H). LRMS (ESI): 510.3 (calc) 511.3 (MH)+ (found). | 303 Method B 508b | | | | |
| 508f | 726f | 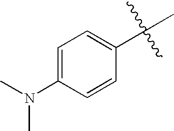 | 4-(2-(2-(4- fluorobenzyloxy) acetamido)ethyl)- N-(4- (dimethylamino) phenyl) piperidine- 1-carboxamide | (DMSO-d₆) δ (ppm) ¹H: 8.10 (s, 1H), 7.80 (t, J=5.7Hz, 1H), 7.44-7.40 (m, 2H), 7.23-7.16 (m, 4H), 6.63 (d, J=9.2Hz, 2H), 4.50 (s, 2H), 4.06 (bd, J=13Hz, 2H), 3.86 (s, 2H), 3.15 (q, J=6.7Hz, 2H), 2.80 (s, 6H), 2.66 (t, J=12Hz, 2H), 1.66 (d, J=12Hz, 2H), | |
| # 1.48-1.33 (m, 3H), 1.05-0.96 (m, 2H). LRMS (ESI): 456.2 (calc) 457.2 (MH)+ (found) | 303 Method A 508a | | | | |
| 508g | 726g | 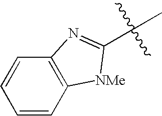 | 4-(2-(2-(4- fluorobenzyloxy) acetamido)ethyl)- N-(1-methyl-1H- benzo[d]imidazol- 2-yl)piperidine-1- carboxamide | (DMSO-d₆) δ (ppm) ¹H: 11.91 (s, 1H), 7.80 (t, J=6.1Hz, 1H), 7.42 (dd, 8.4 and 5.7Hz, 2H), 7.32 (d, J=6.7Hz, 1H), 7.25 (d, J=7.4Hz, 1H), 7.18 (t, J=8.8Hz, 2H), 7.11-7.03 (m, 2H), 4.75-4.60 (m, 1H), 4.50 (s, 2H), 4.42-4.25 (s, 1H), 3.86 (s, 2H), | |
| # 3.45 (s, 3H), 3.15 (q, J=7.2Hz, 2H), 2.82-2.54 (m, 2H), 1.66 (bd, J=11Hz, 2H), 1.50-1.40 (m, 1H), 1.37 (q, J=6.8Hz, 2H), 1.03-0.95 (m, 2H). LRMS (ESI): 467.2 (calc) 468.3 (MH)+(found) | 303 Method D 508d | | | | |
| 508h | 726h | 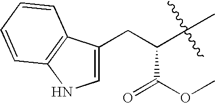 | (S)-methyl 2-(4- (2-(2-(4- fluorobenzyloxy) acetamido)ethyl) piperidine-1- carboxamido)-3- (1H-indol-3- yl)propanoate | (DMSO-d₆) δ (ppm) ¹H: 10.82 (s, 1H), 7.78 (t, J=5.9Hz, 1H), 7.49 (d, J=7.8Hz, 1H), 7.41 (dd, J=8.4 and 5.7Hz, 2H), 7.32 (d, J=7.8Hz, 1H), 7.18 (t, J=9.0Hz, 2H), 7.14 (d, J=2.3Hz, 1H), 7.05 (t, J=6.8Hz, 1H), 6.96 (t, J=7.0Hz, 1H), 6.63 | |
| # (d, J=7.6Hz, 1H), 4.50 (s, 2H), 4.28 (q, J=8.6Hz, 1H), 3.91-3.85 (m, 2H), 3.86 (s, 2H), 3.56 (s, 3H), 3.14-3.02 (m, 4H), 2.66-2.50 (m, 2H), 1.57 (bd, 14Hz, 2H), 1.41-1.29 (m, 3H), 0.98-0.80 (m, 2H). LRMS (ESI): 538.2 (calc) 539.3 (MH)+ (found) | 303 Method D 508d | | | | |
| 508i | 726i | 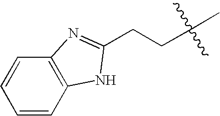 | N-(2-(1H- benzo[d]imidazol- 2-yl)ethyl)-4-(2- (2-(4- fluorobenzyloxy) acetamido)ethyl) piperidine-1- carboxamide | (DMSO-d₆) δ (ppm) ¹H: 8.18 (s, 1H), 7.78 (t, J=5.1Hz, 1H), 7.55-7.32 (m, 4H), 7.18 (t, J=9.0Hz, 2H), 7.09 (dd, J=6.1 and 2.9Hz, 2H), 6.65 (t, J=5.7Hz, 1H), 4.50 (s, 2H), 3.89 (bd, J=13Hz, 2H), 3.86 (s, 2H), 3.44 (q, J=6.3Hz, 2H), 3.12 (q, | |
| # J=6.7Hz, 2H), 2.93 (t, J=7.4Hz, 2H), 2.57 (bt, J=13Hz, 2H), 1.59 (bd, J=12Hz, 2H), 1.42-1.29 (m, 3H), 1.00-0.87 (m, 2H). LRMS (ESI): 481.2 (calc) 482.3 (MH)+ (found) | 303 Method B 508b | | | | |
| 508j | 726j | 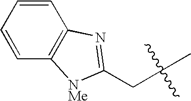 | 4-(2-(2-(4- fluorobenzyloxy) acetamido) ethyl)- N-((1-methyl-1H- benzo[d]imidazol- 2-yl)methyl) piperidine- 1-carboxamide | (DMSO-d₆) δ (ppm) ¹H: 8.14 (s, 1H), 7.79 (t, J=5.9Hz, 1H), 7.55 (d, J=7.8Hz, 1H), 7.49 (d, J=7.4Hz, 1H), 7.42 (dd, J=8.4 and 5.7Hz, 2H), 7.23-7.13 (m, 4H), 7.05 (t, J=5.3Hz, 1H), 4.50-4.46 (m, 4H), 3.96 (bd, J=13Hz, 2H), 3.85 (s, | |
| # 2H), 3.76 (s, 3H), 3.12 (q, J=6.7Hz, 2H), 2.61 (bt, J=12Hz, 2H), 1.61 (bd, J=12Hz, 2H), 1.45-1.32 (m, 3H), 0.96 (bq, J=13Hz, 2H). LRMS (ESI): 481.2 (calc) 482.3 (MH)+ (found) | 303 Method B 508b | | | | |
| 508k | 726k | 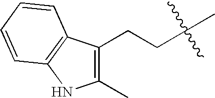 | 4-(2-(2-(4- fluorobenzyloxy) acetamido)ethyl)- N-(2-(2-methyl- 1H-indol-3- yl)ethyl) piperidine- 1-carboxamide | (DMSO-d₆) δ (ppm) ¹H: 10.66 (s, 1H), 7.79 (t, J=5.7Hz, 1H), 7.44-7.40 (m, 3H), 7.18 (t, J=8.6Hz, 3H), 6.94 (t, J=6.8Hz, 1H), 6.89 (t, J=7.4Hz, 1H), 6.50 (t, J=5.3Hz, 1H), 4.50 (s, 2H), 3.91 (db, J=13Hz, 2H), 3.86 (s, 2H), 3.20-3.10 (m, 4H), 2.72 | |
| # (t, J=8.2Hz, 2H), 2.55 (bt, J=12Hz, 2H), 2.29 (s, 3H), 1.60 (bd, J=13Hz, 2H), 1.42-1.32 (m, 3H), 1.00-0.88 (m, 2H). LRMS (ESI): 494.3 (calc) 495.3 (MH)+ (found) | 303 Method B 508b | | | | |
| 508l | 726l | 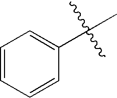 | 4-(2-(2-(4- fluorobenzyloxy) acetamido)ethyl)- N- phenylpiperidine- 1-carboxamide | (DMSO-d₆) δ (ppm) ¹H: 8.41 (s, 1H), 7.81 (t, J=5.7Hz, 1H), 7.44-7.40 (m, 4H), 7.22-7.15 (m, 4H), 6.90 (t, J=7.2Hz, 1H), 4.50 (s, 2H), 4.09 (bd, J=13Hz, 2H), 3.87 (s, 2H), 3.15 (q, J=6.5Hz, 2H), 2.71 (bt, J=11Hz, 2H), 1.68 (bd, J=11Hz, 2H), 1.50-1.34 (m, 3H), 1.02 (qd, J=12 | |
| # and 2.5Hz, 2H). LRMS (ESI): 413.2 (calc) 414.3 (MH)+ (found) | 303 Method C 508c | | | | |
| 508m | 726m | 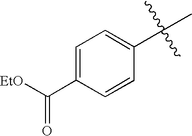 | ethyl 4-(4-(2-(2-(4- fluorobenzyloxy) acetamido)ethyl) piperidine-1- carboxamido) benzoate | (DMSO-d₆) δ (ppm) ¹H: 8.83 (s, 1H), 7.81 (d, J=8.6Hz, 2H), 7.59 (d, J=8.8Hz, 2H), 7.42 (dd, J=8.4 and 5.7Hz, 2H), 7.18 (t, J=9.0Hz, 2H), 4.50 (s, 2H), 4.25 (q, J=7.0Hz, 2H), 4.10 (bd, J=13Hz, 2H), 3.86 (s, 2H), 3.15 (q, J=7.0Hz, 2H), 2.74 (t, J=12Hz, 2H), 1.69 (bd, | |
| # J=11Hz, 2H), 1.50-1.42 (m, 1H), 1.37 (q, J=7.0Hz, 2H), 1.29 (t, J=7.0Hz, 3H), 1.03 (qd, J=13 and 3.5Hz, 2H). LRMS (ESI): 485.2 (calc) 486.3 (MH)+(found) | 303 Method C 508c | | | | |
| 508n | 726n | 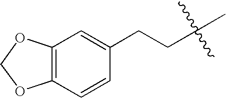 | N-(2- (benzo[d][1,3]dioxol-5-yl) ethyl)-4-(2-(2-(4- fluorobenzyloxy) acetamido)ethyl) piperidine- 1-carboxamide | (DMSO-d₆) δ (ppm) ¹H: 7.78 (t, J=5.7Hz, 1H), 7.42 (dd, J=8.4 and 5.7Hz, 2H), 7.18 (t, J=8.8Hz, 2H), 6.79 (d, J=7.8Hz, 1H), 6.73 (d, J=1.6Hz, 1H), 6.61 (dd, J=8.0 and 1.6Hz, 1H), 6.44 (t, J=5.5Hz, 1H), 5.94 (s, 2H), 4.50 (s, 2H), | |
| # 3.89 (bd, J=15Hz, 2H), 3.86 (s, 2H), 3.19-3.09 (m, 4H), 2.60 (t, J=8.0Hz, 2H), 2.55-2.50 (m, 2H), 1.58 (bd, J=13Hz, 2H), 1.42-1.30 (m, 3H), 0.89 (bq, J=8.2Hz, 2H) LRMS (ESI): 485.2 (calc) 486.3 (MH)+(found) | 303 Method C 508c | | | | |
| 508o | 726o | 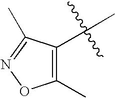 | N-(3,5- dimethylisoxazol- 4-yl)-4-(2-(2-(4- fluorobenzyloxy) acetamido)ethyl) piperidine- 1-carboxamide | (DMSO-d₆) δ (ppm) ¹H: 7.82-7.78 (m, 2H), 7.42 (dd, J=8.6 and 5.7Hz, 2H), 7.18 (t, J=8.8Hz, 2H), 4.50 (s, 2H), 4.00 (bd, J=13Hz, 2H), 3.86 (s, 2H), 3.14 (q, J=6.7Hz, 2H), 2.71 (bt, J=11Hz, 2H), 2.19 (s, 3H), 2.02 (s, 3H), 1.66 (bd, J=11Hz, 2H), | |
| # 1.50-1.40 (m, 1H), 1.37 (q, J=6.8Hz, 2H), 1.01 (qd, J=13 and 4.1Hz, 2H). LRMS (ESI): 432.2 (calc) 433.3 (MH)+ (found) | 303 Method C 508c | | | | |
| 508p | 726p | 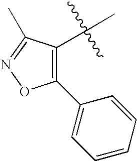 | 4-(2-(2-(4- fluorobenzyloxy) acetamido)ethyl)- N-(3-methyl-5- phenylisoxazol-4- yl)piperidine- 1-carboxamide | (DMSO-d₆) δ (ppm) ¹H:8.17 (s, 1H), 7.82 (t, J=5.9Hz, 1H), 7.77 (d, J=6.8Hz, 1H), 7.54-7.47 (m, 3H), 7.43 (dd, J=8.4 and 5.7Hz, 2H), 7.19 (t, J=8.8Hz, 2H), 4.51 (s, 2H), 4.05 (bd, J=13Hz, 2H), 3.87 (s, 2H), 3.16 (q, J=6.8Hz, 2H), 2.11 (s, 3H), | |
| # 1.69 (bd, J=11Hz, 2H), 1.57-1.42 (m, 1H), 1.39 (q, J=7.0Hz, 2H), 1.03 (qd, J=12 and 2.7Hz, 2H). LRMS (ESI): 494.2 (calc) 495.3 (MH)+ (found) | 303 Method C 508c | | | | |
| 508q | 726q | 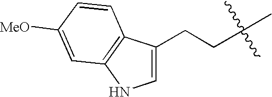 | 4-(2-(2-(4- fluorobenzyloxy) acetamido)ethyl)- N-(2-(6-methoxy- 1H-indol-3- yl)ethyl) piperidine- 1-carboxamide | (DMSO-d₆) δ (ppm) ¹H:10.56 (s, 1H), 7.79 (t, J=5.3Hz, 1H), 7.44-7.37 (m, 3H), 7.18 (t, J=8.8Hz, 2H), 6.95 (d, J=2.2Hz, 1H), 6.81 (d, J=2.2Hz, 1H), 6.62 (dd, J=8.4 and 2.3Hz, 1H), 6.51 (t, J=5.5Hz, 1H), 4.50 (s, 2H), 3.91 (bd, J=13Hz, | |
| # 2H), 3.86 (s, 2H), 3.73 (s, 3H), 3.30-3.21 (m, 2H), 3.13 (q, J=6.3Hz, 2H), 2.74 (t, J=8.0Hz, 2H), 2.57 (bt, J=12Hz, 2H), 1.59 (bd, J=13Hz, 2H), 1.42-1.30 (m, 3H), 1.00-0.92 (m, 2H). LRMS (ESI): 510.3 (calc) 511.3 (MH)+(found) | 303 Method D 508d | | | | |
| 508r | 726r | 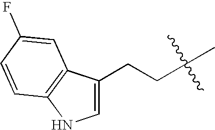 | N-(2-(5-fluoro- 1H-indol-3-yl) ethyl)-4-(2-(2-(4- fluorobenzyloxy) acetamido)ethyl) piperidine- 1-carboxamide | (DMSO-d⁶) δ (ppm) ¹H:10.87 (s, 1H), 7.79 (t, J=5.9Hz, 1H), 7.42 (dd, J=8.4 and 5.7Hz, 2H), 7.31-7.25 (m, 2H), 7.20-7.16 (m, 3H), 6.87 (td, J=9.4 and 2.3Hz, 1H), 6.53 (t, J=5.3Hz, 1H), 4.50 (s, 2H), 3.91 (bd, J=13Hz, 2H), 3.86 (s, 2H), 3.31-3.20 | |
| # (m, 2H), 3.13 (q, J=6.1Hz, 2H), 2.75 (t, J=7.6Hz, 2H), 2.59-2.51 (m, 2H), 1.59 (bd, J=11Hz, 2H), 1.42-1.30 (m, 3H), 1.00-0.88 (m, 2H). LRMS (ESI): 498.2 (calc) 499.3 (MH)+(found) | 303 Method D 508d | | | | |
| 508s | 726s | 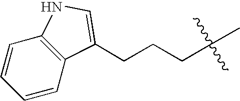 | N-(3-(1H-indol-3- yl)propyl)-4-(2- (2-(4- fluorobenzyloxy) acetamido)ethyl) piperidine- 1-carboxamide | (DMSO-d₆) δ (ppm) ¹H:10.73 (s, 1H), 7.79 (t, J=5.9Hz, 1H), 7.46 (d, J=7.6Hz, 1H), 7.41 (dd, J=8.4 and 5.7Hz, 2H), 7.31 (d, J=8.2Hz, 1H), 7.18 (t, J=9.0Hz, 2H), 7.11 (d, J=2.0Hz, 1H), 7.04 (t, J=8.0Hz, 1H), 6.94 (t, J=7.8Hz, 1H), 6.41 (t, J=5.3Hz, | |
| # 1H), 4.49 (s, 2H), 3.90 (bd, J=13Hz, 2H), 3.85 (s, 2H), 3.12 (q, J=6.3Hz, 2H), 3.06 (q, J=6.5Hz, 2H), 2.64 (t, J=7.4Hz, 2H), 2.55 (bt, J=12Hz, 2H), 1.75 (qt, J=7.2Hz, 2H), 1.59 (bd, J=12Hz, 2H), 1.42-1.30 (m, 3H), 0.92 (bq, J=12Hz, 2H). LRMS (ESI): 494.3 (calc) 495.3 (MH)+ (found) | 303 Method D 508d | | | | |

### EXAMPLE 509a

### 2-(1H-indol-3-yl)ethyl 4-(2-(2-(4-fluorobenzyloxy)acetamido)ethyl)piperidine-1-carboxylate (727a)

To a stirred solution of 1,1′-carbonyldiimidazole (58 mg, 0.36 mmol) in dichloromethane (1 mL) at 0° C. was added a solution of tryptophol (58 mg, 0.36 mmol) in dichloromethane (1.0 mL). The mixture was stirred for 1 h at room temperature. The amine 725 (106 mg, 0.36 mmol) was added, and the reaction stirred for 2 h at room temperature. The solvent was evaporated and the residue was purified by silica gel column chromatography with gradient of methanol (0-10%) in dichloromethane. The collected product was re-purified by prep-hplc with gradient of methanol (20-100%) in water to give 727a (20 mg, 23%).

(DMSO-d₆) δ(ppm) ¹H: 10.83 (s, 1H), 7.78 (t, J=5.7 Hz, 1H), 7.51 (d, J=8.0 Hz, 1H), 7.43-7.38 (m, 2H), 7.31 (d, J=8.0 Hz, 1H), 7.20-7.14 (m, 3H), 7.04 (t, J=6.8 Hz, 1H), 6.95 (t, J=7.0 Hz, 1H), 4.49 (s, 2H), 4.17 (t, J=7.0 Hz, 2H), 4.00-3.85 (m, 2H), 3.85 (s, 2H), 3.11 (q, J=6.7 Hz, 2H), 2.96 (t, J=6.8 Hz, 2H), 2.75-2.60 (m, 2H), 1.65-1.53 (m, 2H), 1.42-1.30 (m, 3H), 1.00-0.83 (m, 2H). LRMS (ESI): (calc) 481.2; (found) 482.1 (MH)⁺

Table 32 presents the characterization data for example 509b prepared according to the method described for compound 727a, example 509a, in scheme 303

TABLE 32

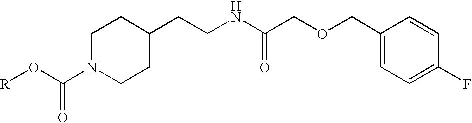

| Ex | Cpd | R | Name | Characterization | Scheme |
|---|---|---|---|---|---|
| 509b | 727b | pyridin-3- ylmethyl 4-(2- (2-(4- fluorobenzyloxy) acetamido)ethyl) piperidine- 1-carboxylate | (DMSO-d₆) δ (ppm) ¹H: 8.67 (s, 1H), 8.63 (d, J=3.7Hz, 1H), 7.91-7.86 (m, 2H), 7.55-7.48 (m, 3H), 7.28 (t, J=8.8Hz, 2H), 5.19 (s, 2H), 4.60 (s, 2H), 4.06 (bd, J=13Hz, 2H), 3.96 (s, 2H), 3.23 (q, J=6.7Hz, 2H), 2.95-2.73 (m, 2H), 1.76 (bd, J=13Hz, 2H), 1.59-1.48 (m, 1H), | | |
| # 1.45 (q, J=7.2Hz, 2H), 7.08 (qd, J=12 and 4.1 Hz, 2H). LRMS (ESI): 429.2 (calc) 430.1 (MH)+ (found) | 303 727a | | | | |

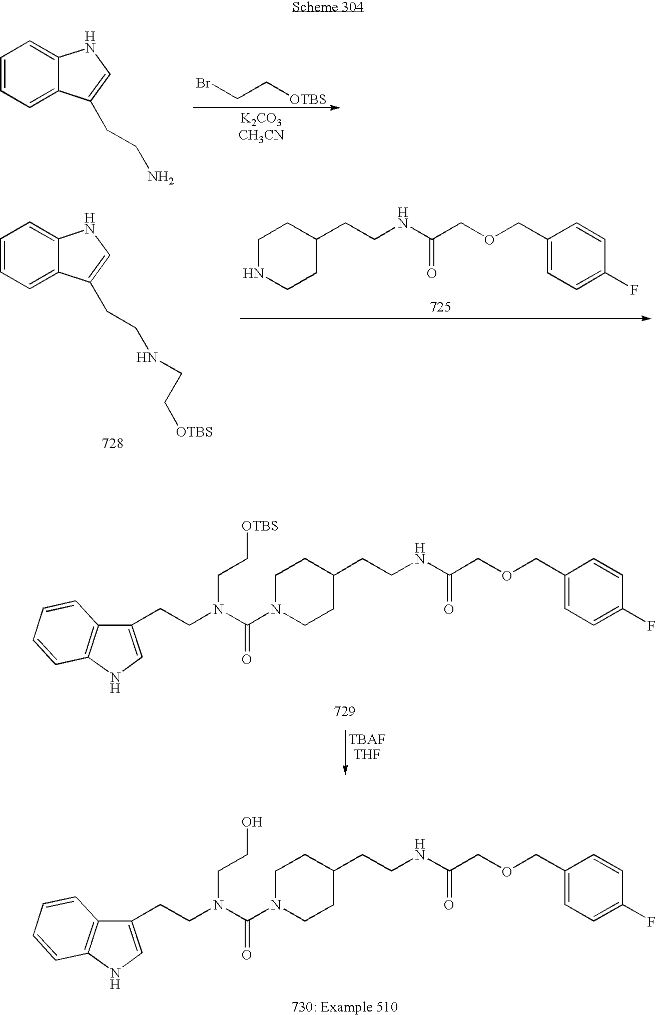

### EXAMPLE 510

### N-(2-(1H-indol-2-yl)ethyl)-4-(2-(2-(4-fluorobenzyloxy)acetamido)ethyl)-N-(2-hydroxyethyl)piperidine-1-carboxamide (730)

### Step 1: N-(2-(1H-indol-3-yl)ethyl)-2-(tert-butyldimethylsilyloxy)ethanamine (728)

To a stirred solution of tryptamine (896 mg, 5.59 mmol) in acetonitrile (23 mL) at 0° C. was added potassium carbonate (1.93 g, 13.98 mmol), followed by (2-bromoethoxy)-tert-butyldimethylsilane (1 mL, 4.66 mmol). The reaction was stirred for 2 h at room temperature, and then for 16 h at 60° C. The solvent was evaporated, a solution of saturated sodium carbonate in water was added, and the residue was extracted with ethyl acetate. The organic extract was dried (MgSO₄), filtered, and evaporated. The residue was purified by silica gel column chromatography with gradient of methanol (0-20%) in dichloromethane to give 728 (560 mg, 38%).

(DMSO-d₆) δ (ppm) ¹H: 10.80 (s, 1H), 7.52 (d, J=8.0 Hz, 1H), 7.33 (d, J=8.2 Hz, 1H), 7.12 (d, J=2.3 Hz, 1H), 7.06 (t, J=7.0 Hz, 1H), 6.97 (t, J=8.0 Hz, 1H), 3.63 (t, J=5.7 Hz, 2H), 3.19 (d, J=3.7 Hz, 1H), 2.83 (s, 4H), 2.65 (t, J=5.7 Hz, 2H), 0.82 (s, 9H), 0.00 (s, 6H). LRMS (ESI): (calc) 318.2; 9found) 319.2 (MH)⁺.

### Step 2: N-(2-(1H-indol-2-yl)ethyl)-N-(2-(tert-butyldimethylsilyloxy)ethyl)-4-(2-(2-(4-fluorobenzyloxy)acetamido)ethyl)piperidine-1-carboxamide (729)

Compound 729 (14 mg, 9%) was prepared starting from 725 (89 mg, 0.28 mmol) and following the same procedures as described for compound 726b, Example 508b, scheme 303, method B,

LRMS (ESI): (calc) 638.4; (found) 639.4 (MH)⁺.

### Step 3: N-(2-(1H-indol-2-yl)ethyl)-4-(2-(2-(4-fluorobenzyloxy)acetamido)ethyl)-N-(2-hydroxyethyl)piperidine-1-carboxamide (730)

To a stirred solution of 729 (14 mg, 0.02 mmol) in THF (0.5 ml) at 0° C. was added tetrabutylammonium fluoride (26 uL of 1M solution in THF, 0.03 mmol). The mixture was stirred for 16 h at room temperature. The solvent was evaporated and the residue was purified by prep-hplc with gradient of methanol (10-100%) in water to give 730 (5 mg, 37%). (MEOD-d₄) δ (ppm) ¹H: 7.55 (d, J=7.8 Hz, 1H), 7.40 (dd, J=8.4 and 5.5 Hz, 2H), 7.31 (d, J=8.2 Hz, 1H), 7.12-7.04 (m, 3H), 7.02-6.97 (m, 2H), 4.56 (s, 2H), 3.92 (s, 2H), 3.64 (t, J=6.1 Hz, 2H), 3.55 (t, J=6.8 Hz, 2H), 3.42-3.35 (m, 4H), 3.21 (t, J=6.8 Hz, 2H), 2.97 (t, J=6.8 Hz, 2H), 2.53 (t, J=11 Hz, 2H), 1.52 (d, J=12 Hz, 2H), 1.40-1.27 (m, 3H), 0.86 (q, J=13 Hz, 2H). LRMS (ESI): (calc) 524.3; (found).525.3 (MH)⁺.

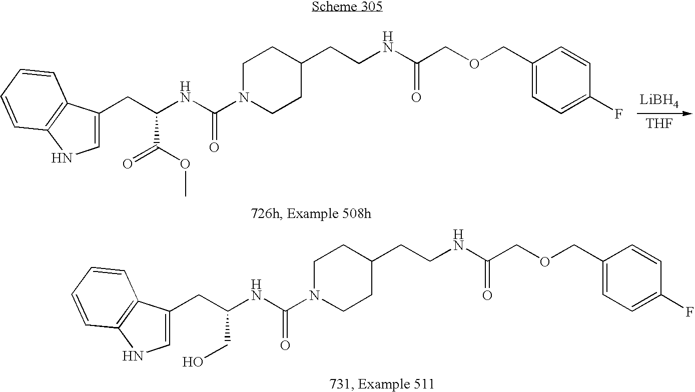

### EXAMPLE 511

### (S)-4-(2-(2-(4-fluorobenzyloxy)acetamido)ethyl)-N-(1-hydroxy-3-(1H-indol-3-yl)propan-2-yl)piperidine-1-carboxamide (731)

To a stirred solution of (S)-methyl 2-(4-(2-(2-(4-fluorobenzyloxy)acetamido)-ethyl)piperidine-1-carboxamido)-3-(1H-indol-3-yl)propanoate 726 h (33 mg, 0.06 mmol) in THF (0.5 mL) was added lithium borohydride (2 mg, 0.07 mmol). The mixture was stirred for 1 h at 50° C. Lithium borohydride was filtered, and the solvent was evaporated. The residue was purified by prep-hplc with gradient of methanol (20-100%) in water to give 731 (15 mg, 48%).

(DMSO-d₆) δ (ppm) ¹H: 10.73 (s, 1H), 7.78 (t, J=6.1 Hz, 1H), 7.60 (d, J=7.4 Hz, 1H), 7.42 (dd, J=8.4 and 5.7 Hz, 2H), 7.29 (d, J=8.0 Hz, 1H), 7.18 (t, J=8.8 Hz, 2H), 7.07 (d, J=2.2 Hz, 1H), 7.03 (t, J=7.0 Hz, 1H), 6.94 (t, J=7.8 Hz, 1H), 5.98 (d, J=7.6 Hz, 1H), 4.64 (t, J=5.5 Hz, 1H), 4.50 (s, 2H), 3.98-3.80 (m, 5H), 3.41-3.33 (m, 1H), 3.12 (q, J=6.1 Hz, 2H), 2.87 (dd, J=14 and 6.5 Hz, 1H), 2.76 (dd, J=14 and 7.4 Hz, 1H), 2.60-2.51 (m, 2H), 1.61-1.53 (m, 2H), 1.40-1.28 (m, 3H), 0.98-0.80 (m, 2H). LRMS (ESI): (calc) 510.2; (found) 511.3 (MH)⁺.

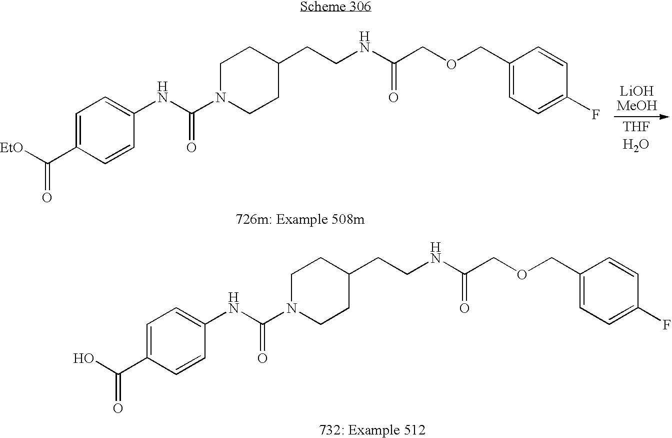

### EXAMPLE 512

### 4-(4-(2-(2-(4-fluorobenzyloxy)acetamido)ethyl)piperidine-1-carboxamido)benzoic acid (732)

To a stirred solution of 726m, example 508, Table 31 (41 mg, 0.08 mmol) in methanol (0.25 mL), water (0.25 mL) and THF (0.25 mL) was added lithium hydroxide monohydrate (18 mg, 0.42 mmol). The reaction was stirred at room temperature for 2 h. Tetrahydrofuran was evaporated, and the residue was purified by prep-hplc with gradient of methanol (20-100%) in water to give 732 (27 mg, 71%).

(DMSO-d₆) δ (ppm) ¹H, 8.77 (s, 1H), 7.81 (t, J=6.1 Hz, 1H), 7.79 (d, J=8.8 Hz, 2H), 7.55 (d, J=8.8 Hz, 2H), 7.42 (dd, J=8.4 and 5.7 Hz, 2H), 7.18 (t, J=8.8 Hz, 2H), 4.50 (s, 2H), 4.09 (bd, J=13 Hz, 2H), 3.86 (s, 2H), 3.15 (q, J=6.1 Hz, 2H), 2.74 (t, J=12 Hz, 2H), 1.69 (bd, J=12 Hz, 2H), 1.50-1.40 (m, 1H), 1.37 (q, J=7.0 Hz, 2H), 1.03 (qd, J=14 and 3.9 Hz, 2H). LRMS (ESI): (calc) 457.2; (found) 458.3 (MH)⁺.

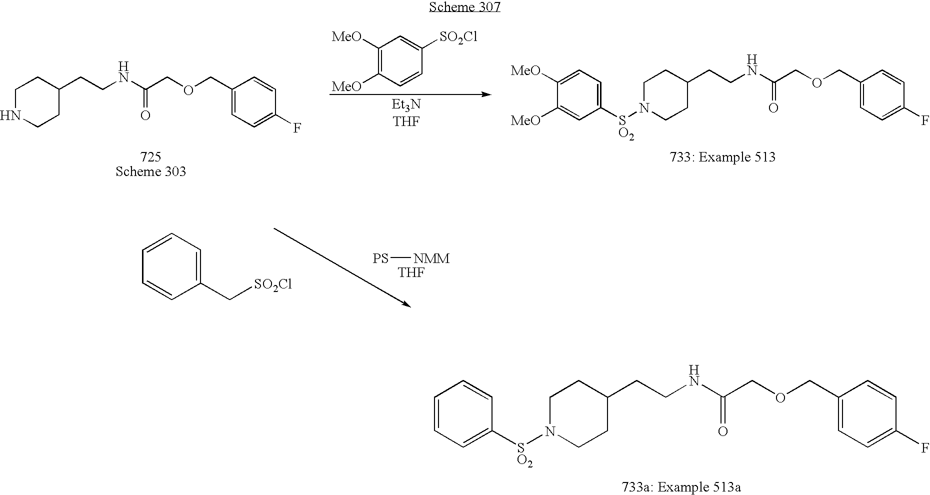

### EXAMPLE 513

### N-(2-(1-(3,4-dimethoxyphenylsulfonyl)piperidin-4-yl)ethyl)-2-(4-fluorobenzyloxy)acetamide (733)

To a stirred solution of 2-(4-fluorobenzyloxy)-N-(2-(piperidin-4-yl)ethyl)acetamide 725, scheme 303 (0.12 mmol) in THF (1.0 mL) was added triethylamine (0.10 mL, 0.69 mmol), followed by 3,4-dimethoxybenzenesulfonyl chloride (29 mg, 0.12 mmol). The mixture was stirred for 16 h at room temperature. More 3,4-dimethoxybenzenesulfonyl chloride (29 mg, 0.12 mmol) was added, and the mixture was stirred for 1 h more. The reaction mixture was quenched with water. The aqueous mixture was extracted with dichloromethane. The organic extract was dried (MgSO₄), filtered, and evaporated. The residue was crystallized from diethyl ether to give 733 (31 mg, 54%). (DMSO-d₆) d(ppm) ¹H, 7.75 (t, J=5.9 Hz, 1H), 7.38 (d, J=5.7 Hz, 1H), 7.36 (d, J=5.7 Hz, 1H), 7.28 (dd, J=8.4 and 2.2 Hz, 1H), 7.16-7.10 (m, 4H), 4.44 (s, 2H), 3.82 (s, 3H), 3.80 (s, 3H), 3.57 (bd, J=12 Hz, 2H), 3.06 (bq, J=6.8 Hz, 2H), 2.10 (bt, J=10 Hz, 2H), 1.69 (bd, J=10 Hz, 2H), 1.29 (bq, J=6.8 Hz, 2H), 1.17-1.04 (m, 3H). LRMS (ESI): 494.2 (calc) 495.0 (MH)+ (found)

### EXAMPLE 513a

### N-(2-(1-(benzylsulfonyl)piperidin-4-yl)ethyl)-2-(4-fluorobenzyloxy)acetamide (733a)

To a stirred solution of PS-NMM (329 mg, 0.75 mmol) in DCM (2.5 mL) was added 2amine 725 (83 mg, 0.25 mmol) followed by α-toluenesulfonyl chloride (44 mg, 0.23 mmol). The reaction was stirred at room temperature for 16 h. More α-toluenesulfonyl chloride (44 mg, 0.23 mmol) was added, and the mixture stirred for 1 h at room temperature, and 1.5 h at 50° C. PS-NMM was filtered, solvent evaporated and the residue was purified by prep-hplc with gradient of methanol (20-100%) in water to give 733a (19 mg, 18%).

(DMSO-d₆) δ (ppm) ¹H, 7.80 (t, J=5.7 Hz, 1H), 7.44-7.33 (m, 7H), 7.18 (t, J=9.0 Hz, 2H), 4.50 (s, 2H), 4.35 (s, 2H), 3.85 (s, 2H), 3.50 (bd, J=12 Hz, 2H), 3.11 (q, J=6.8 Hz, 2H), 2.60 (td, J=12 and 2.3 Hz, 2H), 1.67 (bd, J=1 Hz, 2H), 1.40-1.23 (m, 3H), 1.02 (qd, J=12 and 4.1 Hz, 2H). LRMS (ESI): (calc) 448.2; (found) 449.2 (MH)⁺.

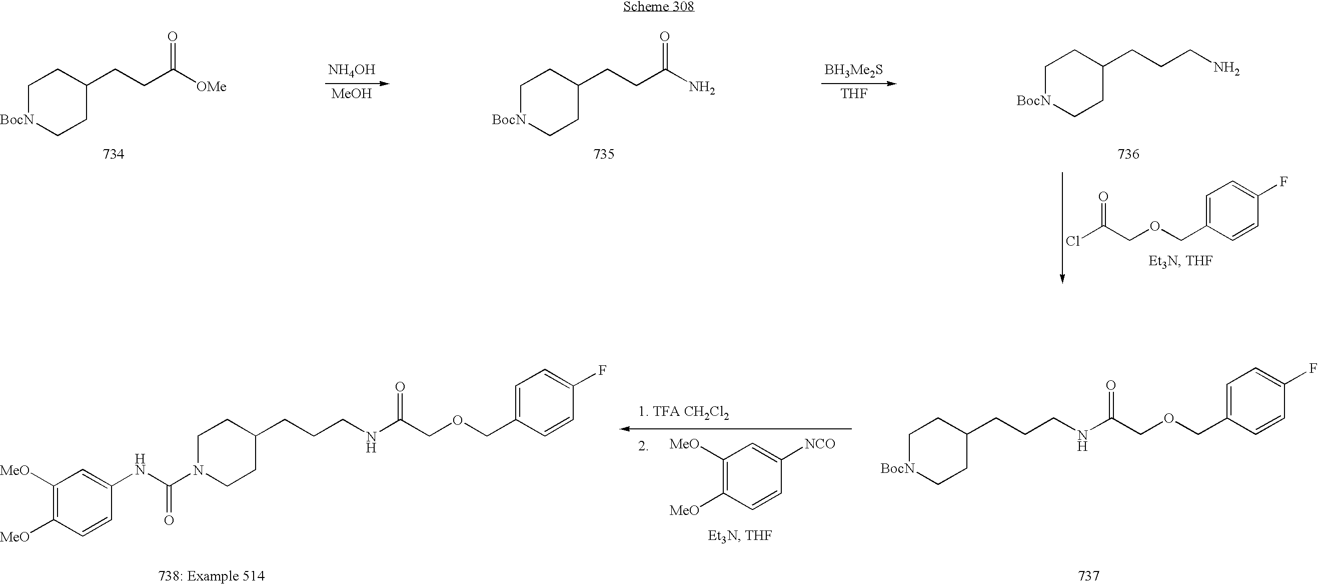

### EXAMPLE 514

### 4-(3-(2-(4-fluorobenzyloxy)acetamido)propyl)-N-(3,4-dimethoxyphenyl)piperidine-1-carboxamide (738)

### Step 1: tert-butyl 4-(3-amino-3-oxopropyl)piperidine-1-carboxylate (735)

Ammonium hydroxide (3 mL of 28% in H₂O) was added to a stirred solution of tert-butyl 4-(3-methoxy-3-oxopropyl)piperidine-1-carboxylate 734 (646 mg, 2.38 mmol, prepared from N-boc-4-piperidinemethanol by oxidation followed by the sequence desribed by Klein, Scott I. et al.; J. Med. Chem. 1998, 41; 14; 2492-2502) in methanol (3 mL). The mixture was stirred for 45 min at room temperature, and then for 16 h at 50° C. The solvent was evaporated. The residue was purified by silica gel column chromatography with gradient of methanol (0-10%) in DCM to give 735 (490 mg, 80%).

(DMSO-d₆) δ (ppm) ¹H, 7.23 (s, 1H), 6.69 (s, 1H), 3.88 (bd, J=9.8 Hz, 2H), 2.78-2.50 (m, 2H), 2.03 (t, J=7.2 Hz, 2H), 1.58 (bd, J=12 Hz, 2H), 1.44-1.20 (m, 3H), 1.36 (s, 9H), 0.91 (qd, J=12 and 4.3 Hz, 2H). LRMS (ESI): (calc) 256.2; (found) 256.0 (M)⁺.

### Step 2: tert-butyl 4-(3-aminopropyl)piperidine-1-carboxylate (736)

To 735 (490 mg, 1.91 mmol) was added borane methyl sulfide complex (7.6 mL of a 2M solution in THF, 15.3 mmol). The mixture was stirred at 60° C. for 4 h. The reaction mixture was quenched with a saturated solution of sodium carbonate in water. The aqueous mixture was extracted with dichloromethane. The organic extract was dried (MgSO₄), filtered, and evaporated. The crude product was used without purification.

### Step 3: tert-butyl 4-(3-(2-(4-fluorobenzyloxy)acetamido)propyl)piperidine-1-carboxylate (737)

Compound 736 (0.96 mmol) in THF (3 mL) and triethylamine (0.25 mL, 1.8 mmol), was reacted with 2-(4-fluorobenzyloxy)acetyl chloride (0.90 mmol) in THF (1.5 mL) as described for the synthesis of compound 723, example 507, step 4, scheme 302. Purification by silica gel column chromatography with gradient of ethyl acetate (60-80%) in hexane gave 737 (65 mg, 17%).

LRMS (ESI): (calc) 408.2; (found) 431.1 (M+Na)⁺.

### Step 4: 4-(3-(2-(4-fluorobenzyloxy)acetamido)propyl)-N-(3,4-dimethoxyphenyl)piperidine-1-carboxamide (738)

To a stirred solution of 737 (65 mg, 0.16 mmol) in DCM (0.7 mL) was added trifluoroacetic acid (0.1 mL). The mixture was stirred for 4.5 h at room temperature. The solvent was evaporated giving the crude amine, 2-(4-fluorobenzyloxy)-N-(3-(piperidin-4-yl)propyl)acetamide. LRMS (ESI): 308.2 (calc) 309.1 (MH)⁺ (found).

To the crude amine (0.16 mmol) in THF (1 mL) at 0° C. was added triethylamine (0.07 mL, 0.48 mmol), then 3,4-dimethoxyphenylisocyanate (24 uL, 0.16 mmol). The reaction was stirred at room temperature for 16 h. More 3,4-dimethoxyphenylisocyanate (18 uL, 0.12 mmol) was added. The reaction was stirred at room temperature for 1 h. The reaction mixture was quenched with brine. The aqueous mixture was extracted with dichloromethane. The organic extract was dried (MgSO₄), filtered, and evaporated. The residue was purified by silica gel column chromatography with gradient of methanol (0-5%) in dichloromethane to give 738 (33 mg, 42%).

(DMSO-d₆) δ (ppm) ¹H, 8.24 (s,1H), 7.79 (t, J=5.5 Hz, 1H), 7.41 (dd, J=8.6 and 5.7 Hz, 2H), 7.19-7.13 (m, 3H), 6.94 (dd, J=8.6 and 2.5 Hz, 1H), 6.78 (d, J=8.8 Hz, 1H), 4.49 (s, 2H), 4.07 (bd, J=13 Hz, 2H), 3.85 (s, 2H), 3.67 (s, 3H), 3.66 (s, 3H), 3.07 (q, J=6.5 Hz, 2H), 2.65 (bt, J=12 Hz, 2H), 1.64-1.61 (m, 2H), 1.45-1.39 (m, 3H), 1.19-1.16 (m, 2H), 1.02-0.93 (m, 2H). LRMS (ESI): (calc) 487.2; (found) 488.1 (MH)⁺.

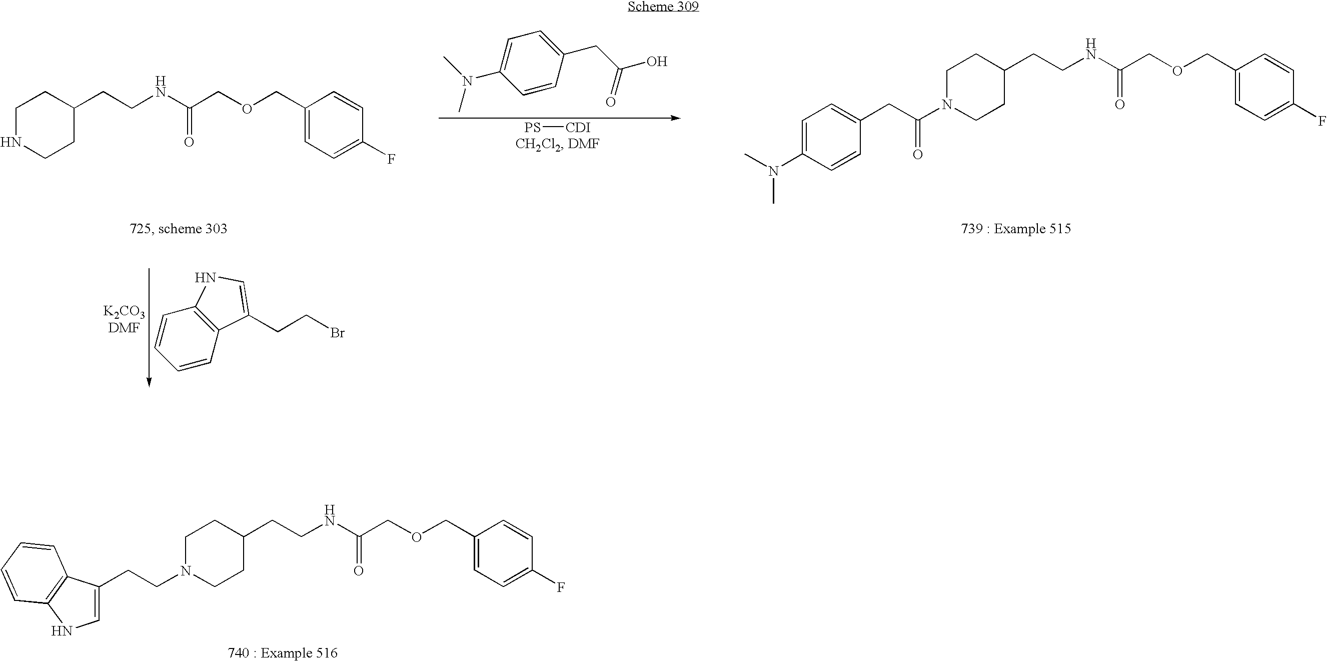

### EXAMPLE 515

### N-(2-(1-(2-(4-(dimethylamino)phenyl)acetyl)piperidin-4-yl)ethyl)-2-(4-fluorobenzyloxy)acetamide (739)

To a stirred solution of PS-CDI (424 mg, 0.5 mmol) in DCM (2.5 mL) was added 4-(dimethylamino)phenylacetic acid (67 mg, 0.37 mmol), and the mixture was stirred for 10 min. Then a solution of amine 725 (83 mg, 0.25 mmol) in DMF (1 mL) was added. The reaction was stirred at room temperature for 16 h. PS-CDI was filtered, solvent evaporated and the residue was purified by prep-hplc with gradient of methanol (20-100%) in water to give 739 (36 mg, 32%).

(DMSO-d₆) δ (ppm) ¹H, 7.77 (t, J=5.7 Hz, 1H), 7.41 (dd, J=8.2 and 5.7 Hz, 2H), 7.17 (t, J=8.8 Hz, 2H), 7.01 (d, J=8.6 Hz, 2H), 6.65 (d, J=8.8 Hz, 2H), 4.49 (s, 2H), 4.33 (bd, J=13 Hz, 1H), 3.88 (bd, J=113 Hz, 1H), 3.84 (s, 2H), 3.52 (s, 2H), 3.10 (q, J=6.8 Hz, 2H), 2.89-2.84 (m, 1H), 2.83 (s, 6H), 2.50-2.41 (m, 1H), 1.65-1.56 (M, 2H), 1.44-1.40 (m, 1H), 0.83 (sxd, 12 and 3.9 Hz, 2H). LRMS (ESI): (calc) 455.3; (found) 456.3 (MH)⁺.

### EXAMPLE 516

### N-(2-(1-(2-(1H-indol-3-yl)ethyl)piperidin-4-yl)ethyl)-2-(4-fluorobenzyloxy)acetamide (740)

To a stirred solution of amine 725 (50 mg, 0.17 mmol) in dimethylformamide (1.7 mL) was added potassium carbonate (94 mg, 0.68 mmol) followed by 3-(2-bromoethyl)indole (38 mg, 0.17 mmol). The reaction was stirred at 90° C. for 2 h. The reaction mixture was quenched with a saturated solution of sodium carbonate in water. The aqueous mixture was extracted with ethyl acetate, and the organic extract was dried (MgSO₄), filtered, and evaporated. The residue was purified by silica gel column chromatography with gradient of methanol (0-10%) in dichloromethane to give 740 (25 mg, 34%).

(DMSO-d₆) δ (ppm) ¹H: (10.76 (s, 1H), 7.77 (t, J=5.9 Hz, 1H), 7.48 (d, J=7.8 Hz, 1H), 7.41 (dd, J=8.6 and 5.7 Hz, 2H), 7.30 (d, J=8.0 Hz, 1H), 7.17 (t, J=9.0 Hz, 2H), 7.12 (s, 1H), 7.03 (t, J=7.8 Hz, 1H), 6.94 (t, J=7.0 Hz, 1H), 4.49 (s, 2H), 3.85 (s, 2H), 3.12 (q, J=7.0 Hz, 2H), 3.02-2.90 (m, 2H), 2.90-2.78 (m, 2H), 2.65-2.50 (m, 2H), 2.05-1.80 (m, 2H), 1.73-1.60 (m, 2H), 1.35 (q, J=7.0 Hz, 2H), 1.31-1.08 (m, 3H). LRMS (ESI): (calc) 437.2; (found) 438.1 (MH)⁺.

Example 515a compound 740a was prepared as describer for Example 515, compound 739, and scheme 309. Characterization data is presented in Table 33

Examples 516a-b, compounds 740a-b, were prepared as described for Example 516, compound 740. Characterization data is presented in Table 33.

TABLE 33

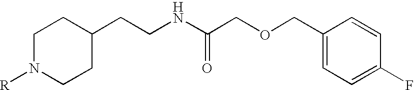

| Ex | Cpd | R | Name | Characterization | Scheme |
|---|---|---|---|---|---|
| 515a | 739a | 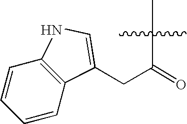 | N-(2-(1-(2-(1H- indol-3-yl)acetyl) piperidin-4- yl)ethyl)-2-(4- fluorobenzyloxy) acetamide | (DMSO-d₆) δ (ppm) ¹H: 10.86 (s, 1H), 7.76 (t, J=5.7Hz, 1H), 7.54 (d, J=8.0Hz, 1H), 7.40 (t, J=8.4Hz, 2H), 7.32 (d, J=8.2Hz, 1H), 7.20-7.14 (m, 3H), 7.05 (t, J=7.0Hz, 1H), 6.95 (t, J=6.8Hz, 1H), 4.48 (s, 2H), 4.36 (bd, J=13Hz, 1H), 3.98 (bd, J=13Hz, 1H), 3.84 | |
| # (s, 2H), 3.72 (s, 2H), 3.08 (q, J=6.7Hz, 2H), 2.88 (bt, J=12Hz, 1H), 2.50-2.43 (m, 1H), 1.64-1.54 (m, 2H), 1.50-1.34 (m, 1H), 1.26 (q, J=7.4Hz, 2H), 0.90-0.73 (m, 2H). LRMS (ESI): (calc) 451.2; (found) 452.3 (MH)⁺. | 309 Ex 515 | | | | |
| 516a | 740a | 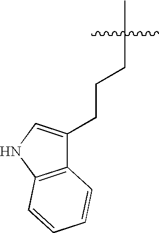 | N-(2-(1-(3-(1H- indol-3- yl)propyl) piperidin-4- yl)ethyl)-2-(4- fluorobenzyloxy) acetamide | (DMSO-d⁶) δ (ppm) ¹H: 10.73 (s, 1H), 7.77 (t, J=5.7Hz, 1H), 7.49-7.39 (m, 2H), 7.30 (d, J=8.0Hz, 1H), 7.17 (t, J=9.0Hz, 2H), 7.08 (d, J=2.2Hz, 1H), 7.03 (t, J=7.6Hz, 1H), 6.94 (t, J=7.8Hz, 1H), 4.49 (s, 2H), 3.85 (s, 2H), 3.11 (q, J=7.0Hz, 2H), 2.88-2.79 | |
| # (m, 2H), 2.66 (t, J=7.4Hz, 2H), 2.38-2.24 (m, 2H), 1.88-1.70 (m, 4H), 1.62 (bd, J=11Hz, 2H), 1.34 (q, J=7.2Hz, 2H), 1.25-1.05 (m, 3H). LRMS(ESI): (calc) 451.3; (found) 452.3 (MH)⁺ | 309 Ex 516 | | | | |
| 516b | 740b | 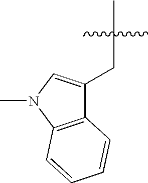 | 2-(4- fluorobenzyloxy) -N-(2-(1-((1- methyl-1H-indol- 3-yl)methyl) piperidin-4- yl)ethyl) acetamide | (DMSO-d₆) δ (ppm) ¹H: 8.47 (s, 1H), 7.70 (d, J=8.0Hz, 1H), 7.46 (d, J=10Hz, 1H), 7.44 (s, 1H), 7.39 (dd, J=8.4 and 5.7Hz, 2H), 7.28 (t, J=7.2Hz, 1H), 7.19 (t, J=8.0Hz, 1H), 7.07 (t, J=8.6Hz, 1H), 4.55 (s, 2H), 4.45 (s, 2H), 3.91 (s, 2H), 3.86 (s, 3H), 3.51 | |
| # (bd, J=12Hz, 2H), 3.26 (t, J=7.2Hz, 2H), 3.02-2.93 (m, 2H), 1.98 (bd, J=13Hz, 2H), 1.61-1.32 (m, 5H). LRMS: 437.2 (calc) 438.3 (MH)+(found) | 309 Ex 516 | | | | |

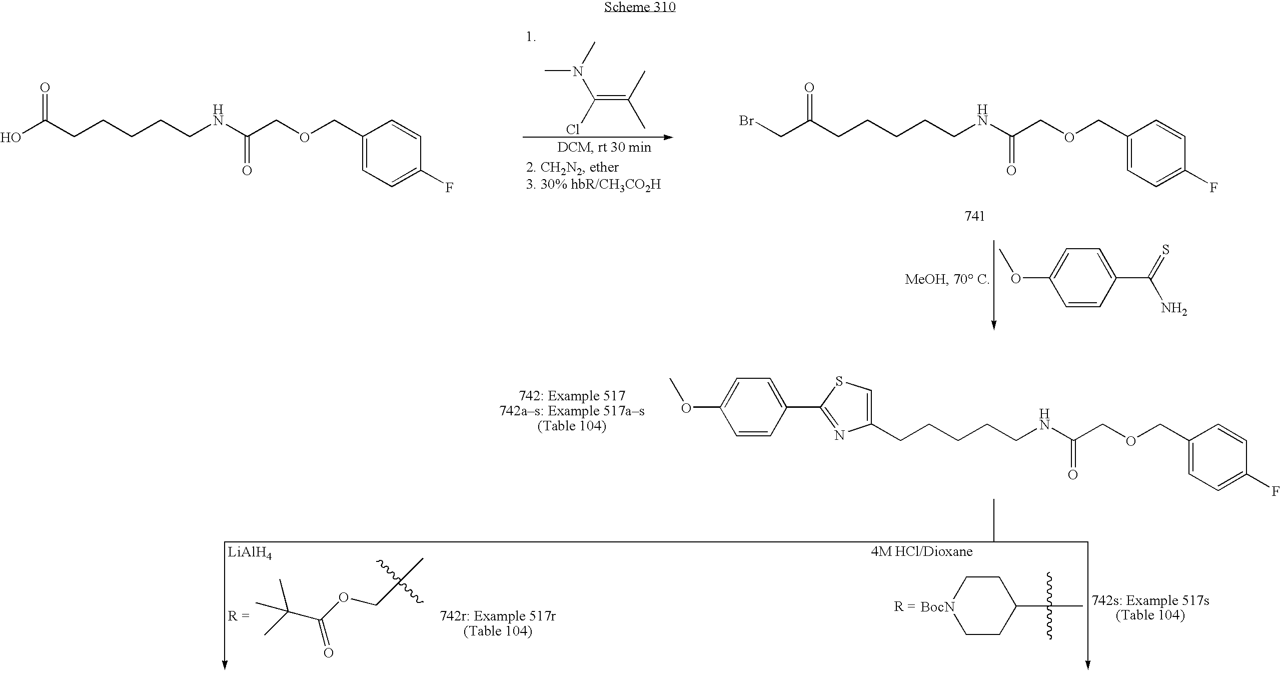

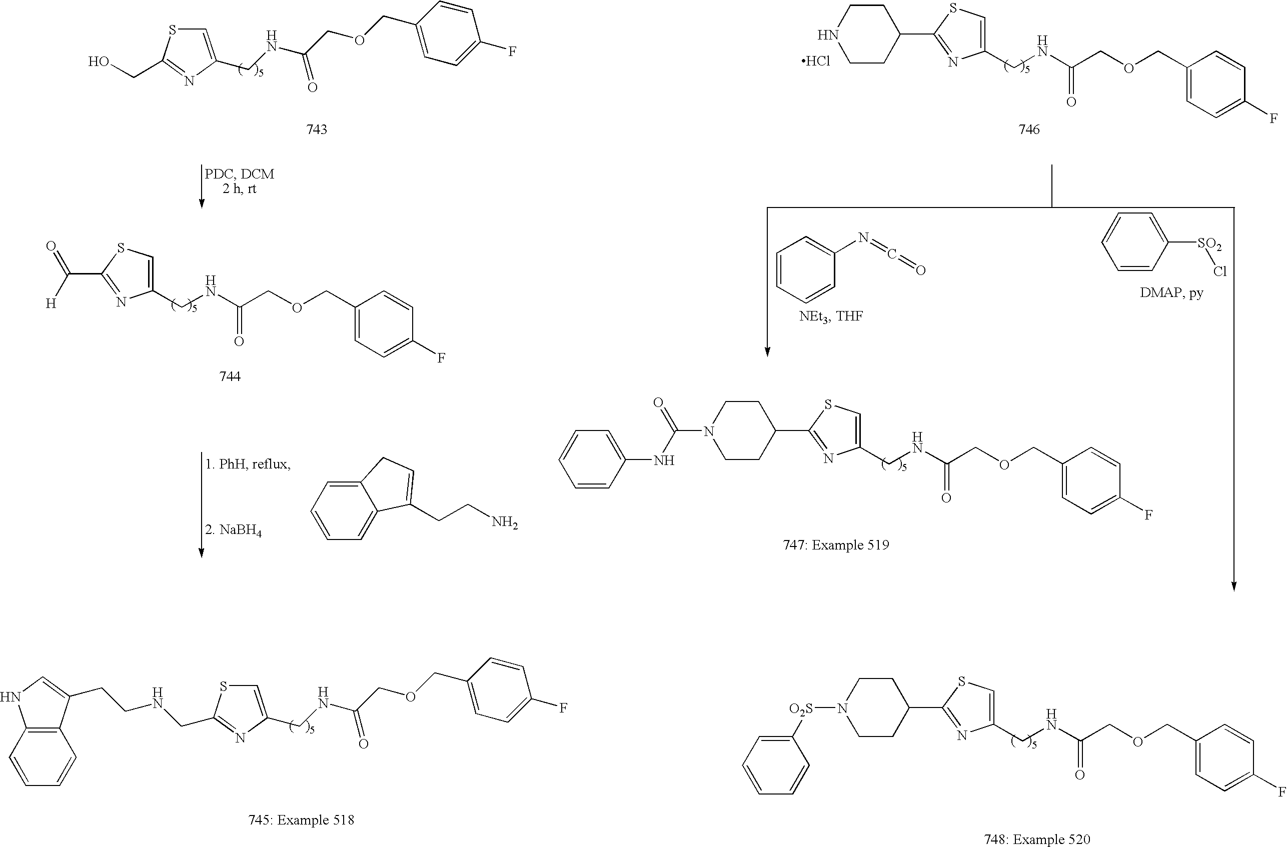

### EXAMPLE 517

### 2-(4-fluorobenzyloxy)-N-(5-(2-(4-methoxyphenyl)thiazol-4-yl)pentyl)acetamide (742)

### Step 1: 2: N-(7-bromo-6-oxoheptyl)-2-(4-fluorobenzyloxy)acetamide (741)

1-Chloro-N,N,2-trimethyl-1-propenylamine (0.450 mL, 3.39 mmol) was added to a solution of 6-(2-(4-fluorobenzyloxy)acetamido)hexanoic acid (842 mg, 2.83 mmol, as described for the synthesis of compound 303, Example 112a, step 3,scheme 49) in DCM (30 mL). The reaction stirred for 1 h at room temperature. The solvent was evaporated and crude 6-(2-(4-fluorobenzyloxy)acetamido)hexanoyl chloride, was diluted with 10 mL of diethyl ether. Diazomethane (0.5M, 14.1 mmol) in diethyl ether (from N-nitroso-N-methylurea, 1.45 g) was added to the reaction at 0° C. and reaction mixture was stirred for 30 min. A solution of hydrogen bromide in acetic acid (1.14 mL, 4.24 mmol) was added drop-wise at 0° C. and the orange mixture stirred for 2 h. The solvent was evaporated and the residue was purified by silica gel column chromatography with ethyl acetate (60%) in hexane to give 741 (850 mg, 81%) as white crystals. The product was contaminated with N,N-dimethylisobutyramide and was used as is without further purification.

(CD₃CN) δ (ppm) ¹H, 7.44 (m, 2H), 7.16-7.11 (m, 2H), 6.85 (bs, 1H), 4.54 (s, 2H), 4.09 (s, 2H), 3.90 (m, 2H), 3.20 (q, J=6.8 Hz, 2H), 2.60 (t, J=7.2 Hz, 2H), 1.61-1.56 (m, 2H), 1.50-1.45 (m, 2H), 1.33-1.29 (m, 2H).

LRMS (ESI): (calc) 374.2; (found) 376.1 (MH)⁺.

### Step 2: 2-(4-fluorobenzyloxy)-N-(5-(2-(4-methoxyphenyl)thiazol-4-yl)pentyl)acetamide (742)

4-Methoxybenzothioamide (73 mg, 0.439 mmol) was added to a solution of 741 (137 mg, 0.366 mmol) in methanol (5 mL). The reaction was heated to 70° C. and stirred for 16 h. The solvent was evaporated and the residue was purified by silica gel column chromatography with gradient of ethyl acetate (40%-60%) in hexane to give 742 (20 mg, 12%) as a clear oil.

¹H NMR: (CDCl₃) d(ppm): 7.86 (d, J=8.8 Hz, 2H),7.29 (dd, J=6.0,2.4 Hz, 2H), 7.05 (t, J=8.6 Hz, 2H), 6.93 (d, J=8.8 Hz, 2H), 6.78 (s, 1H), 6.54 (bs, 1H), 4.51 (s, 2H), 3.95 (s, 2H), 3.85 (s, 3H), 3.30 (q, J=6.8 Hz, 2H), 2.79 (t, J=7.4 Hz, 2H), 1.82-1.74 (m, 2H), 1.60-1.54 (m, 2H), 1.45-1.40 (m, 2H).

LRMS (ESI): 442.6 (calc) 443.1 (MH)+ (found).

Examples 517a-s describe the preparation of compounds 742a-s using the same procedures as described for compound 742, Example 517, scheme 310. Characterization data are presented in Table 34.

TABLE 34

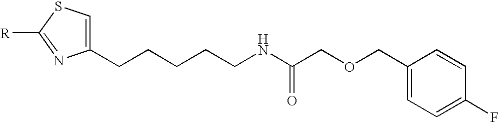

| Ex | Cpd | R₃ | Name | Characterization | Scheme |
|---|---|---|---|---|---|
| 517a | 742a | 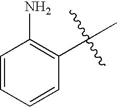 | N-(5-(2-(2- aminophenyl) thiazol-4-yl) pentyl)-2-(4- fluorobenzyloxy) acetamide | (CDCl₃) δ (ppm): 7.60 (dd, J=8.2, 1.4Hz, 1H), 7.29-7.26 (m, 2H), 7.16-7.12 (m, 1H), 7.06-7.02 (m, 2H), 6.75-6.66 (m, 3H), 6.54 (bs, 1H), 6.16 (bs, 2H), 4.49 (s, 2H), 3.94 (s, 2H), 3.29 (q, J=6.8Hz, 2H), 2.78 (t, J=7.4Hz, 2H), 1.81-1.73 (m, 2H), 1.59-1.53 (m, 2H), | |
| # 1.44-1.36 (m, 2H). | 310 Step 1-4 Ex 517 | | | | |
| 517b | 742b | 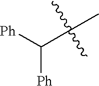 | N-(5-(2- benzhydrylthiazol- 4-yl)pentyl)-2-(4- fluorobenzyloxy) acetamide | (CDCl₃) δ (ppm): 7.32-7.21 (m, 12H), 7.07-7.03 (m, 2H), 6.79 (s, 1H), 6.53 (bs, 1H), 5.81 (s, 1H), 4.51 (s, 2H), 3.94 (s, 2H), 3.27 (q, J=6.8Hz, 2H), 2.75 (t, J=7.6Hz, 2H), 1.75-1.67 (m, 2H), 1.58-1.50 (m, 2H), 1.40-1.35 (m, 2H). | 310 Step 1-4 Ex 517 |
| 517c | 742c | 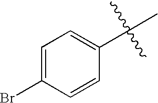 | N-(5-(2-(4- bromophenyl) thiazol-4-yl) pentyl)-2-(4- fluorobenzyloxy) acetamide | (CDCl₃) δ (ppm): 7.82 (d, J=6.8, 2Hz, 2H), 7.55 (d, J=6.8, 2Hz, 2H), 7.30-7.27 (m, 2H), 7.07-7.03 (m, 2H), 6.54 (bs, 1H), 4.51 (s, 2H), 3.30 (quartet, J=6.8Hz, 2H), 2.83 (t, J=7.8Hz, 2H), 1.78 (quintet, J=7.6Hz, 2H), 1.60-1.54 (m, 2H), 1.45-1.40 (m, 2H). | 310 Step 1-4 Ex 517 |
| 517d | 742d | 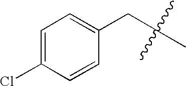 | N-(5-(2-(4- chlorobenzyl) thiazol-4- yl)pentyl)-2-(4- fluorobenzyloxy) acetamide | (CDCl₃) δ (ppm): 7.30-7.27 (m, 3H), 7.23-7.21 (m, 2H), 7.07-7.03 (m, 2H), 6.73 (s, 1H), 6.54 (bs, 1H), 4.51 (s, 2H), 4.25 (s, 2H), 3.95 (s, 2H), 3.27 (quartet, J=6.8Hz, 2H), 2.73 (t, J=7.6Hz, 2H), 1.75-1.67 (m, 2H), 1.57-1.51 (m, 2H), 1.41-1.35 (m, 2H). | 310 Step 1-4 Ex 517 |
| 517e | 742e | 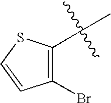 | N-(5-(2-(3- bromothiophen- 2-yl)thiazol-4- yl)pentyl)-2-(4- fluorobenzyloxy) acetamide | (CDCl₃) δ (ppm): 7.31-7.26 (m, 3H), 7.07-7.03 (m, 3H), 6.93 (s, 1H), 6.55 (bs, 1H). 4.51 (s, 2H), 3.95 (s, 2H), 3.30 (q, J=6.8Hz, 2H), 2.80 (t, J=7.4Hz, 2H), 1.80-1.72 (m, 2H), 1.60-1.53 (m, 2H), 1.44-1.38 (m, 2H). | 310 Step 1-4 Ex 517 |
| 517f | 742f | 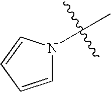 | N-(5-(2-(4-(1H- pyrrol-1- yl)phenyl)thiazol- 4-yl)pentyl)-2-(4- fluorobenzyloxy) acetamide | (CDCl₃) δ (ppm): 7.99 (t, J= 1.8Hz, 1H), 7.77 (dt, J=5.7, 1.2Hz, 1H), 7.49-7.41 (m, 2H), 7.30-7.26 (m, 2H), 7.17 (t, J=2.0Hz, 2H), 7.07-7.02 (m, 2H), 6.91 (s, 1H), 6.54 (bs, 1H), 6.37 (t, J=2.4Hz, 2H), 4.51 (s, 2H), 3.95 (s, 2H), 3.31 (q, J=7.2Hz, 2H), 2.83 (t, J=7.6Hz, | |
| # 2H), 1.84-1.76 (m, 2H), 1.62-1.55 (m, 2H), 1.46-1.39 (m, 2H). | 310 Step 1-4 Ex 517 | | | | |
| 517g | 742g | 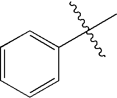 | 2-(4- fluorobenzyloxy)- N-(5-(2- phenylthiazol-4- yl)pentyl) acetamide | (CDCl₃) δ (ppm) ¹H: 7.94-7.90 (m, 2H), 7.44-7.39 (m, 3H), 7.30-7.25 (m, 2H), 7.08-7.02 (m, 2H), 6.86 (s, 1H), 6.54 (bs, 1H), 4.50 (s, 2H), 3.95 (s, 2H), 3.30 (q, J=6.8Hz, 2H), 2.82 (t, J=7.6Hz, 2H), 1.83-1.75 (m, 2H), 1.62-1.54 (m, 2H), 1.46-1.40 (m, 2H). | 310 Step 1-4 Ex 517 |
| 517h | 742h | 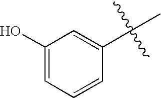 | 2-(4- fluorobenzyloxy)- N-(5-(2-(3- hydroxyphenyl) thiazol-4-yl) pentyl)acetamide | (CDCl₃) δ (ppm) ¹H: 7.69 (bs, 1H), 7.62-7.61 (m, 1H), 7.33-7.31 (m, 1H), 7.27-7.22 (m, 3H), 7.05-7.01 (m, 2H), 6.90-6.88 (m, 1H), 6.84 (s, 1H), 6.66 (bs, 1H), 4.48 (s, 2H), 3.97 (s, 2H), 3.36 (q, J=6.8Hz, 2H), 2.81 (t, J=7.2Hz, 2H), 1.76 (quintet, J=7.6Hz, 2H), 1.67-1.61 | |
| # (m, 2H), 1.46-1.40 (m, 2H). | 310 Step 1-4 Ex 517 | | | | |
| 517i | 742i | 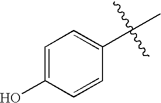 | 2-(4- fluorobenzyloxy)- N-(5-(2-(4- hydroxyphenyl) thiazol-4-yl) pentyl)acetamide | (CDCl₃) δ (ppm) ¹H: 8.40 (bs, 1H), 7.72 (d, J=8.8Hz, 2H), 7.28-7.25 (m, 2H), 7.06-7.01 (m, 2H), 6.80 (d, J=8.8Hz, 2H), 6.61 (bs, 1H), 4.49 (s, 2H), 3.95 (s, 2H), 3.28 (q, J=7.2Hz, 2H), 2.77 (t, J=7.2Hz, 2H), 1.77-1.70 (m, 2H), 1.58-1.51 (m, 2H), 1.41-1.35 (m, | |
| # 2H). | 310 Step 1-4 Ex 517 | | | | |
| 517j | 742j | 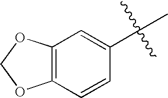 | N-(5-(2- (benzo[d][1,3]dioxol-5- yl)thiazol-4- yl)pentyl)-2-(4- fluorobenzyloxy) acetamide | (CDCl₃) δ (ppm) ¹H: 7.43-7.40 (m, 2H), 7.30-7.26 (m, 2H), 7.06-7.02 (m, 2H), 6.82 (d, J=8Hz, 1H), 6.78 (s, 1H), 6.55 (bs, 1H), 6.00 (s, 2H), 4.50 (s, 2H), 3.94 (s, 2H), 3.29 (q, J=6.8Hz, 2H), 2.77 (t, J=7.6Hz, 2H); 1.80-1.72 (m, 2H), 1.59-1.53 | |
| # (m, 2H), 1.44-1.38 (m, 2H). | 310 Step 1-4 Ex 517 | | | | |
| 517k | 742k | 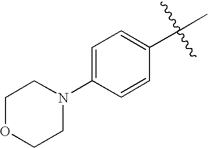 | 2-(4- fluorobenzyloxy)- N-(5-(2-(4- morpholinophenyl) thiazol-4-yl) pentyl)acetamide | (CDCl₃) δ (ppm) ¹H: 7.82 (d, J=8.8Hz, 2H), 7.30-7.25 (m, 2H), 7.07-7.03 (m, 2H), 6.90 (d, J=9.2Hz, 2H), 6.76 (s, 1H), 6.54 (bs, 1H), 4.50 (s, 2H), 3.95 (s, 2H), 3.88-3.85 (m, 4H), 3.29 (q, J=7.2Hz, 2H), 3.24-3.22 (m, 4H), 2.79 (t, J=7.6Hz, 2H), 1.81-1.73 (m, | |
| # 2H), 1.60-1.54 (m, 2H), 1.45-1.39 (m, 2H). | 310 Step 1-4 Ex 517 | | | | |
| 517l | 742l | 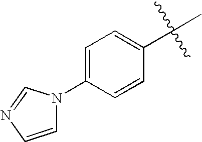 | N-(5-(2-(4-(1H- imidazol-1- yl)phenyl)thiazol- 4-yl)pentyl)-2-(4- fluorobenzyloxy) acetamide | (CDCl₃) δ (ppm) ¹H: 8.03 (d, J=8.8Hz, 2H), 7.90 (s, 1H), 7.44 (d, J=8.8Hz, 2H), 7.32 (s, 1H), 7.30-7.26 (m, 2H), 7.22 (s, 1H), 7.07-7.02 (m, 2H), 6.91 (s, 1H), 6.56 (bs, 1H), 4.51 (s, 2H), 3.95 (s, 2H), 3.30 (q, J=6.8Hz, 2H), 2.82 (t, J=7.6 Hz, 2H), 1.83-1.75 | |
| # (m, 2H), 1.62-1.54 (m, 2H), 1.46-1.38 (m, 2H). | 310 Step 1-4 Ex 517 | | | | |
| 517m | 742m | 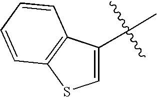 | N-(5-(2- (benzo[b]thiophen- 3-yl)thiazol-4- yl)pentyl)-2-(4- fluorobenzyloxy) acetamide | (CDCl₃) δ (ppm) ¹H: 8.70-8.68 (m, 1H), 7.93 (s, 1H), 7.89-7.87 (m, 1H), 7.51-7.47 (m, 1H), 7.43-7.39 (m, 1H), 7.28-7.25 (m, 2H), 7.06-7.01 (m, 2H), 6.88 (s, 1H), 6.56 (bs, 1H), 3.31 (q, J=6.8Hz 2H), 2.87 (t, J=7.6Hz, 2H), 1.88-1.80 (m, 2H), 1.64-1.56 | |
| # (m, 2H), 1.49-1.41 (m, 2H). | 310 Step 1-4 Ex 517 | | | | |
| 517n | 742n | 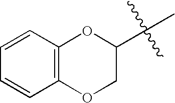 | N-(5-(2-(2,3- dihydrobenzo[b][1,4]dioxin-2- yl)thiazol-4-yl) pentyl)-2-(4- fluorobenzyloxy) acetamide | (CDCl₃) δ (ppm) ¹H: 7.31-7.27 (m, 2H), 7.07-6.99 (m, 3H), 6.94-6.88 (m, 3H), 6.55 (bs, 1H), 5.48 (dd, J=7.4, 2.8Hz, 1H), 4.60 (dd, J=11.4, 2.8Hz, 1H), 4.52 (s, 2H), 4.22 (dd, J=11.6, 7.2Hz, 1H), 3.95 (s, 2H), 3.28 (q, J=6.8Hz, 2H), 2.77 (t, | |
| # J=8.0Hz, 2H), 1.77-1.69 (m, 2H), 1.58-1.52 (m, 2H), 1.41-1.36 (m, 2H). | 310 Step 1-4 Ex 517 | | | | |
| 517o | 742o | 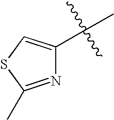 | 2-(4- fluorobenzyloxy)- N-(5-(2′-methyl- 2,4′-bithiazol-4- yl)pentyl)acetamide | (CDCl₃) δ (ppm) ¹H: 7.76 (s, 1H), 7.30-7.26 (m, 2H), 7.08-7.03 (m, 2H), 6.88 (s, 1H), 6.54 (bs, 1H), 4.51 (s, 2H), 3.95 (s, 2H), 3.29 (q, J=7.2Hz, 2H), 2.80 (t, J=7.6Hz, 2H), 2.76 (s, 3H), 1.81-1.73 (m, 2H), 1.61-1.53 (m, 2H). | 310 Step 1-4 Ex 517 |
| 517p | 742p | 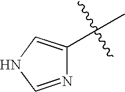 | N-(5-(2-(1H- imidazol-4- yl)thiazol-4- yl)pentyl)-2-(4- fluorobenzyloxy) acetamide | (MEOD-d₄) δ (ppm) ¹H: 9.01 (d, J=1.2Hz, 1H), 8.11 (d, J=1.2Hz, 1H), 7.40-7.36 (m, 2H), 7.34 (s, 1H), 7.09-7.05 (m, 2H), 4.54 (s, 2H), 3.91 (s, 2H), 3.23 (t, J=7.2Hz, 2H), 2.84 (t, J=7.6Hz, 2H), 1.83-1.76 (m, 2H), 1.60-1.53 (m, 2H), 1.42-1.32 (m, 2H). | 310 Step 1-4 Ex 517 |
| 517q | 742q | 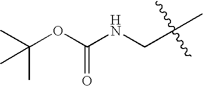 | tert-butyl (4-(5- (2-(4- fluorobenzyloxy) acetamido)pentyl) thiazol-2-yl) methylcarbamate | (CD₃CN) δ (ppm) ¹H: 7.43-7.39 (m, 2H), 7.16-7.11 (m, 2H), 6.94 (s, 1H), 6.85 (bs, 1H), 6.05 (bs, 1H), 4.54 (s, 2H), 4.43 (d, J=6.0Hz, 2H), 3.90 (s, 2H), 3.20 (q, J=6.8Hz, 2H), 2.70 (t, J=7.6Hz, 2H), 2.17 (s, 1H), 1.72 (m, 2H), 1.55-1.48 (m, 2H), 1.44 (s, 9H), 1.37-1.32 | |
| # (m, 2H). | 310 Step 1-4 Ex 517 Except used ethanol as solvent In step 4 | | | | |
| 517r | 742r | 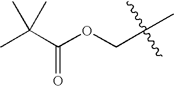 | (4-(5-(2-(4- fluorobenzyloxy) acetamido)pentyl) thiazol-2-yl) methyl pivalate | (CD₃CN) δ (ppm) ¹H: 7.30-7.25 (m, 2H), 7.07-7.03 (m, 2H), 6.86 (s, 1H), 6.54 (bs, 1H), 5.34 (s, 2H), 4.51 (s, 2H), 3.94 (s, 2H), 3.28 (q, J=6.8Hz, 2H), 2.74 (t, J=7.8Hz, 2H), 1.75-1.68 (m, 2H), 1.58-1.51 (m, 2H), 1.41-1.33 (m, 2H), 1.24 (s, 9H). | 310 Step 1-4 Ex |
| # 517 Except used ethanol as solventin step 4 | | | | | |
| 517s | 742s | 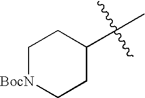 | tert-butyl 4-(4-(5- (2-(4- fluorobenzyloxy) acetamido)pentyl) thiazol-2-yl) piperidine-1- carboxylate | (CDCl₃) δ (ppm) ¹H: 7.31-7.27 (m, 2H), 7.07-7.03 (m, 2H), 6.73 (s, 1H), 6.55 (bs, 1H), 4.52 (s, 2H), 4.18 (bs, 2H), 3.95 (s, 2H), 3.27 (q, J=7.2Hz, 2H), 3.13-3.07 (m, 1H), 2.87-2.81 (m, 2H), 2.72 (t, J=7.6Hz, 2H), 2.07-2.04 (m, 2H), 1.74-1.63 | |
| # (m, 3H), 1.58-1.51 (m, 2H), 1.46 (s, 9H), 1.40-1.35 (m, 2H). | 310 Step 1-4 Ex 517 | | | | |

### EXAMPLE 518

### N-(5-(2-((2-(1H-indol-3-yl)ethylamino)methyl)thiazol-4-yl)pentyl)-2-(4-fluorobenzyloxy)acetamide (745)

### Step 1: 2-(4-fluorobenzyloxy)-N-(5-(2-(hydroxymethyl)thiazol-4-yl)pentyl)acetamide (743)

A solution of compound 742r, scheme 310, example 517r, (800 mg, 1.77 mmol) in THF (2 mL) was added to a solution of LiAlH₄ (131 mg, 3.55 mmol) in THF (2 mL) at room temperature. The reaction was stirred for 10 min., and then it was cooled to 0° C. and slowly quenched with H₂O until the grey mixture turned white. The reaction was diluted with 10% HCl in water. The aqueous mixture was extracted with ethyl acetate. The organic extract was dried (Na₂SO₄), filtered, and evaporated. The alcohol, 743 was used without further purification (615 mg, 95%, clear oil).

(MEOD-d₄) δ(ppm) ¹H, 7.88 (bs, 1H), 7.42-7.38 (m, 2H), 7.42 (s, 1H), 7.41-7.38 (m, 2H), 4.88 (s, 2H), 4.79 (s,1H), 4.55 (s, 2H), 3.92 (s, 2H), 3.24-3.20 (m, 2H), 2.72 (t, J=7.4 Hz, 1H), 1.72-1.67 (m, 1H), 1.58-1.50 (m, 3H), 1.38-1.31 (m, 3H).

LRMS (ESI): (calc) 366.4; (found) 367.1 (MH)⁺.

### Step 2: 2-(4-fluorobenzyloxy)-N-(5-(2-formylthiazol-4-yl)pentyl)acetamide (744)

Pyridinium dichromate (1.26 g, 3.36 mmol) was added to a solution of 743 (615 mg, 1.60 mmol) in dichloromethane (1 ml). The reaction stirred at room temperature for 5 h. The mixture was filtered over a pad of silica, washing with ethyl acetate. The filtrate was evaporated and the residue was purified by silica gel column chromatography with gradient of methanol (0%-2%) in ethyl acetate to give 744 (100 mg, 17%) as a clear film.

LRMS (ESI): (calc) 364.4; (found) 387.1 (MNa)⁺.

### Step 3: N-(5-(2-((2-(1H-indol-3-yl)ethylamino)methyl)thiazol-4-yl)pentyl)-2-(4-fluorobenzyloxy)acetamide (745)

Tryptamine (87 mg, 0.548 mmol) and 744 (100 mg, 0.274 mmol) in benzene (5 ml, the flask equipped with a condenser and a Dean-Stark trap) was heated to 90° C. for 16 h. The reaction was cooled to room temperature and sodium triacetoxyborohydride (58 mg, 0.274 mmol) was added. The mixture stirred for 3 h at room temperature. The reaction was diluted with 10% NaOH in water and the aqueous mixture was extracted with ethyl acetate and the organic layer was dried (Na₂SO₄), filtered, and evaporated. The residue was first purified by prep-hplc with gradient of methanol (20%-95%) in water, and the product was re-purified by prep-TLC plate, eluting with methanol (4%) in DCM to give 745 (6 mg, 4%) as a yellow film.

(CDCl₃) δ(ppm) ¹H, 7.40 (d, J=8.0 Hz, 1H), 7.30-7.26 (m, 2H), 7.12 (d, J=8.0 Hz, 1H), 6.99-6.94 (m, 4H), 6.88-6.85 (m, 4H), 4.43 (s, 2H), 3.97 (s, 2H), 3.80 (s, 2H), 3.09 (t, J=7.0 Hz, 2H), 2.87 (s, 3H), 2.60 (t, J=7.4 Hz, 2H), 1.62-1.54 (m, 2H), 1.46-1.38 (m, 2H), 1.26-1.18 (m, 2H).

LRMS (ESI): (calc) 508.6; (found) 509.3 (MH)⁺.

### EXAMPLE 519

### 4-(4-(5-(2-(4-fluorobenzyloxy)acetamido)pentyl)thiazol-2-yl)-N-phenylpiperidine-1-carboxamide (747)

### Step 1: 2-(4-fluorobenzyloxy)-N-(5-(2-(piperidin-4-yl)thiazol-4-yl)pentyl)acetamide (746)

Compound 742s (570 mg, 1.09 mmol, scheme 310, example 517s) was dissolved in a minimum amount of DCM. A solution of HCl (4M, 10.9 mmol) in ether was added to the solution and the reaction stirred at room temperature for 16 h. The solvent was evaporated and the hydrochrloride salt 746 was triturated with ether to give the product as a white solid. LRMS (ESI): (calc) 456.0; (found) 457.0 (MH)⁺.

### Step 2: 4-(4-(5-(2-(4-fluorobenzyloxy)acetamido)pentyl)thiazol-2-yl)-N-phenylpiperidine-1-carboxamide (747)

Phenylisocyanate (52 mg, 0.438 mmol) was added to a solution of 746 (100 mg, 0.219 mmol) and triethylamine (0.057 mL, 0.438 mmol) in THF (2 mL) and the reaction stirred at room temperature for 16 h, then it was diluted with a saturated solution of sodium carbonate in water. The aqueous mixture was extracted with EtOAc and the extracts were dried (Na₂SO₄), filtered, and evaporated. The residue was purified by silica gel column chromatography with gradient of methanol (1%-10%) in diethyl ether to give 747 (60 mg, 51%) as a clear oil.

(CDCl₃) δ(ppm) ¹H: 7.37-7.34 (m, 2H), 7.31-7.25 (m, 2H), 7.07-7.00 (m, 3H), 6.79 (s, 1H), 6.59-6.56 (m, 2H), 4.51 (s, 2H), 4.19-4.15 (m, 2H), 3.94 (s, 2H), 3.27 (q, J=6.8 Hz, 3H), 3.07-3.00 (m, 2H), 2.75 (t, J=7.6 Hz, 2H), 2.18-2.16 (m, 2H), 1.85-1.68 (m, 4H), 1.59-1.51 (m, 2H), 1.42-1.33 (m, 2H). LRMS: (calc) 538.6; (found) 539.1 (MH)⁺.

### EXAMPLE 520

### 2-(4-fluorobenzyloxy)-N-(5-(2-(1-(phenylsulfonyl)piperidin-4-yl)thiazol-4-yl)pentyl)acetamide (748)

Phenylsulfonylchloride (77 mg, 0.438 mmol) was added to a solution of 746, scheme 310 (100 mg, 0.219 mmol) and catalytic amounts of dimethylaminopyridine in pyridine (2 mL). The reaction stirred at room temperature for 16 h. The reaction was diluted with 10% HCl in water. The aqueous mixture was extracted with ethyl acetate, and the organic extract was dried (Na₂SO₄), filtered, and evaporated. The residue was purified by silica gel column chromatography with pure diethyl ether to give 748 (15 mg, 12%) as clear oil.

(CDCl₃) δ (ppm) ¹H: 7.81-7.79 (m, 2H), 7.70-7.60 (m, 3H), 7.41-7.60 (m, 2H), 7.10-7.05 (m, 2H), 6.98 (s, 1H), 4.55 (s, 2H), 3.91 (s, 2H), 3.85-3.82 (m, 2H), 3.20 (t, J=7.2 Hz, 2H), 2.96-2.90 (m, 2H), 2.69 (t, J=7.6 Hz, 2H), 2.46 (td, J=12, 2.4 Hz, 2H), 2.11-2.09 (m, 2H), 1.84-1.76 (m, 2H), 1.71-1.63 (m, 2H), 1.56-1.50 (m, 2H), 1.39-1.30 (m, 2H).

LRMS (ESI): (calc) 559.7; (found) 560.0 (MH)⁺.

Example 519a describes the preparation of compound 747a using the same procedure as described for compound 747 in Example 519, scheme 310. Characterization data is presented in Table 35.

Examples 520a-b describe the preparation of compounds 748a-b using the same procedures as described for compound 748 in Example 520, scheme 310. Characterization data are presented in Table 35.

TABLE 35

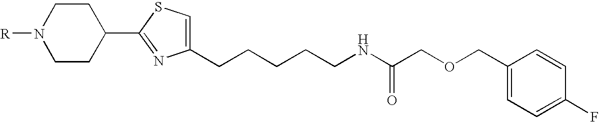

| Ex | Cpd | R₃ | Name | Characterization | Scheme |
|---|---|---|---|---|---|
| 519a | 747a | 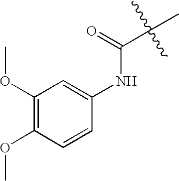 | N-(3,4- dimethoxyphenyl)- 4-(4-(5-(2-(4- fluorobenzyloxy) acetamido)pentyl) thiazol-2-yl) piperidine-1- carboxamide | (CDCl₃) δ (ppm) ¹H: 7.30-7.27 (m, 2H), 7.07-7.02 (m, 2H), 6.77 (s, 1H), 6.64 (s, 1H), 6.63 (d, J=2.4Hz, 2H), 6.56 (bs, 1H), 6.15 (t, J=2.0Hz, 1H), 4.51 (s, 2H), 4.17-4.13 (m, 2H), 3.94 (s, 2H), 3.75 (s, 6H), 3.29-3.23 (m, 3H), 3.06-2.99 | |
| # (m, 2H), 2.73 (t, J=7.6Hz, 2H), 2.17-2.14 (m, 2H), 1.84-1.67 (m, 4H), 1.58-1.50 (m, 2H), 1.42-1.32 (m, 2H). | 310 Ex 519 | | | | |
| 520a | 748a | 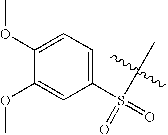 | N-(5-(2-(1-(3,4- dimethoxyphenyl sulfonyl)piperidin- 4-yl)thiazol-4- yl)pentyl)-2-(4- fluorobenzyloxy) acetamide | (CDCl₃) δ (ppm) ¹H: 7.39 (dd, J=8.4, 2.4Hz, 1H), 7.30-7.27 (m, 2H), 7.22 (d, J=2.4Hz, 1H), 7.07-7.02 (m, 2H), 6.96 (d, J=8.8Hz, 1H), 6.75 (s, 1H), 6.53 (bs, 1H), 4.51 (s, 2H), 3.95 (s, 2H), 3.94 (s, 3H), 3.93 (s, 3H), 3.87-3.84 (m, 2H), 3.26 (q, | |
| # J=6.8Hz, 2H), 2.96-2.94 (m, 1H), 2.70 (t, J=7.6Hz, 2H), 2.43 (td, J=12, 2.4Hz, 2H), 2.17-2.14 (m, 2H), 1.94-1.83 (m, 2H), 1.72-1.64 (m, 2H), 1.57-1.50 (m, 2H), 1.39-1.33 (m, 2H). | 310 Ex 520 | | | | |
| 520b | 748b | 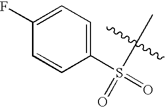 | 2-(4- fluorobenzyloxy)- N-(5-(2-(1-(4- fluorophenyl- sulfonyl)piperidin- 4-yl)thiazol-4- yl)pentyl)acetamide | (CDCl₃) δ (ppm) ¹H: 7.81-7.77 (m, 2H), 7.31-7.27 (m, 2H), 7.24-7.20 (m, 2H), 7.07-7.03 (m, 2H), 6.75 (s, 1H), 6.54 (bs, 1H), 4.52 (s, 2H), 3.94 (s, 2H), 3.87-3.84 (m, 2H), 3.26 (q, J=6.8Hz, 2H), 2.98-2.93 (m, 1H), 2.70 (t, J=7.6Hz, 2H), | |
| # 2.44 (td, J=12, 2.4Hz, 2H), 2.18-2.15 (m, 2H), 1.94-1.84 (m, 2H), 1.72-1.64 (m, 2H), 1.57-1.50 (m, 2H), 1.39-1.32 (m, 2H). | 310 Ex 520 | | | | |

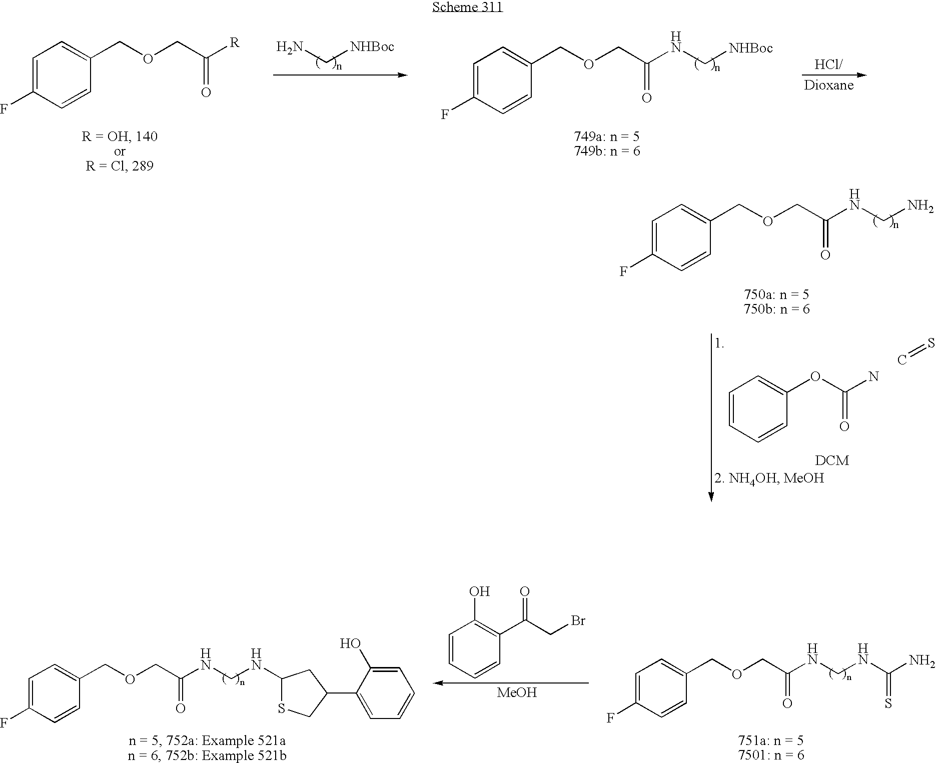

### EXAMPLES 521a

### 2-(4-fluorobenzyloxy)-N-(5-(4-(2-hydroxyphenyl)thiazol-2-ylamino)pentyl)acetamide (752a)

### Step 1: tert-butyl 5-(2-(4-fluorobenzyloxy)acetamido)pentylcarbamate (749a)

A solution of acid chloride 289 (2.71 mmol) in THF (3 mL) was added to a solution of N-boc-1,5-diaminopentane (0.376 mL, 1.80 mmol) and diisopropylethylamine (0.627 mL, 3.60 mmol) in THF (3 mL). The reaction stirred at room temperature for 1 h. The reaction was diluted with 10% HCl in water. The aqueous mixture was extracted with ethyl acetate. The organic extract was dried (Na₂SO₄), filtered, and evaporated. The crude product 749a, an orange solid, was used without further purification (883 mg, 88% crude). LRMS (ESI): (calc) 368.4; (found) 369.0 (MH)⁺.

### Step 2: N-(5-aminopentyl)-2-(4-fluorobenzyloxy)acetamide (750a)

A solution of dry HCl (4M, 7.19 mmol) in dioxane was added to a solution of 749a in dioxane (1.80 mL). The reaction stirred at room temperature for 1 h. The reaction was quenched with a saturated solution of sodium carbonate in water. The aqueous mixture was extracted with ethyl acetate. The organic extract was dried (Na₂SO₄), filtered, and evaporated. The crude product 750a, yellow oil, was used without further purification (420 mg, 65% crude).

LRMS (ESI): (calc) 268.3; (found) 269.0 (MH)⁺.

### Step 3: 2-(4-fluorobenzyloxy)-N-(5-thioureidopentyl)acetamide (751a)

Benzoyl isothiocyanate (0.126 mL, 0.938 mmol) was added to a mixture 750a (210 mg, 0.782 mmol) in DCM (3 mL) and the reaction stirred at room temperature for 1 h. Solvent was evaporated and the residue was dissolved in methanol (2 mL). Ammonium hydroxide was added (1 mL) and the reaction stirred for 16 h. The reaction was diluted with 10% HCl in water. The aqueous mixture was extracted with ethyl acetate. The organic extract was dried (Na₂SO₄), filtered, and evaporated. The crude product 751a, brown oil, was used without further purification (255 mg, 99% crude).

LRMS (ESI): (calc) 327.4; (found) 328.0 (MH)⁺.

### Step 4: 2-(4-fluorobenzyloxy)-N-(5-(5-(2-hydroxyphenyl)thiazol-2-ylamino)pentyl)acetamide (752a)

2-Bromo-1-(2-hydroxyphenyl)ethanone (201 mg, 0.938 mmol) was added to a solution of 751a (255 mg, 0.782 mmol) in methanol (5 mL). The reaction was heated to 70° C. and stirred for 16 h. The solvent was evaporated and the residue was purified by silica gel column chromatography with gradient of ethyl acetate (50%-70%) in hexane to give 752a (39 mg, 11% over 4 steps) as an opaque oil.

(CDCl3) δ(ppm): 12.0 (bs, 1H), 7.52 (dd, J=7.6, 1.6 Hz, 1H), 7.30-7.27 (m, 2H), 7.20-7.15 (m, 1H), 7.06-7.02 (m, 2H), 6.93 (dd, J=8.4, 1.2 Hz, 1H), 6.84-6.80 (m, 1H) 6.65 (s, 1H), 6.63 (bs, 1H), 5.61-5.58 (m, 1H), 4.52 (s, 2H), 3.97 (s, 2H), 3.34-3.28 (m, 4H), 1.69 (quintet, J=5.4 Hz, 2H), 1.56 (quintet, J=5.4 Hz, 2H), 1.45-1.39 (m, 2H).

LRMS (ESI): (calc) 443.5; (found) 444.1 (MH)⁺.

### EXAMPLE 521b

### 2-(4-fluorobenzyloxy)-N-(6-(4-(2-hydroxyphenyl)thiazol-2-ylamino)hexyl)acetamide (752b)

### Step 1: tert-butyl 6-(2-(4-fluorobenzyloxy)acetamido)hexylcarbamate (749b)

Prepared by the reaction of acid 140 (500 mg, 2.71 mmol) and EDC (779 mg, 4.06 mmol) in dimethylformamide (6 mL). After 5 min, N-boc-1,6-aminohexane (753 mg, 2.98 mmol) and catalytic amount of DMAP was added to the solution and the reaction was stirred for 32 h. The mixture was diluted with 10% HCl in water and the aqueous layer was extracted with ethyl acetate. The organic extract was dried (Na₂SO₄), filtered, and evaporated. The residue was purified by silica gel column chromatography with diethyl ether (70%) in hexane to give 749b (1.03 mg, 28%) as a white solid.

LRMS (ESI): (calc) 382.4; (found) 405.0 (MNa)⁺.

### Step 2: N-(6-aminohexyl)-2-(4-fluorobenzyloxy)acetamide (750b)

Was prepared following the same procedure as described for compound 750a (scheme 311, example 521a) to give 750b (611 mg, 100% crude, yellow oil).

LRMS (ESI): (calc) 282.3; (found) 283.0 (MH)⁺.

### Step 3: 2-(4-fluorobenzyloxy)-N-(6-thioureidohexyl)acetamide (751b)

Was prepared following the same procedure as described for compound 751a (scheme 311, Example 521a) to give 751b (263 mg, 100% crude) as yellow solid.

### Step 4: 2-(4-fluorobenzyloxy)-N-(6-(4-(2-hydroxyphenyl)thiazol-2-ylamino)hexyl)acetamide (752b)

Was prepared following the same procedure as described for compound 752a (scheme 311, example 521a) except that the residue was purified by silica gel column chromatography with gradient of ethyl acetate (40%-70%) in hexane to give 752b (8 mg, 2% yield over 4 steps) as a clear oil.

(CDCl3) δ (ppm) ¹H: 7.52 (dd, J=7.6, 1.6 Hz, 1H), 7.31-7.27 (m, 2H), 7.21-7.16 (m, 1H), 7.07-7.02 (m, 2H), 6.97-6.82 (m, 1H), 6.68 (s, 1H), 6.60 (bs, 1H), 6.15 (bs, 1H), 4.52 (s, 2H), 3.97 (s, 2H), −3.34-3.27 (m, 4H), 1.73-1.66 (m, 2H), 1.57-1.50 (m, 2H), 1.48-1.33 (m, 4H).

LRMS (ESI): (calc) 457.5; (found) 458.0 (MH)⁺.

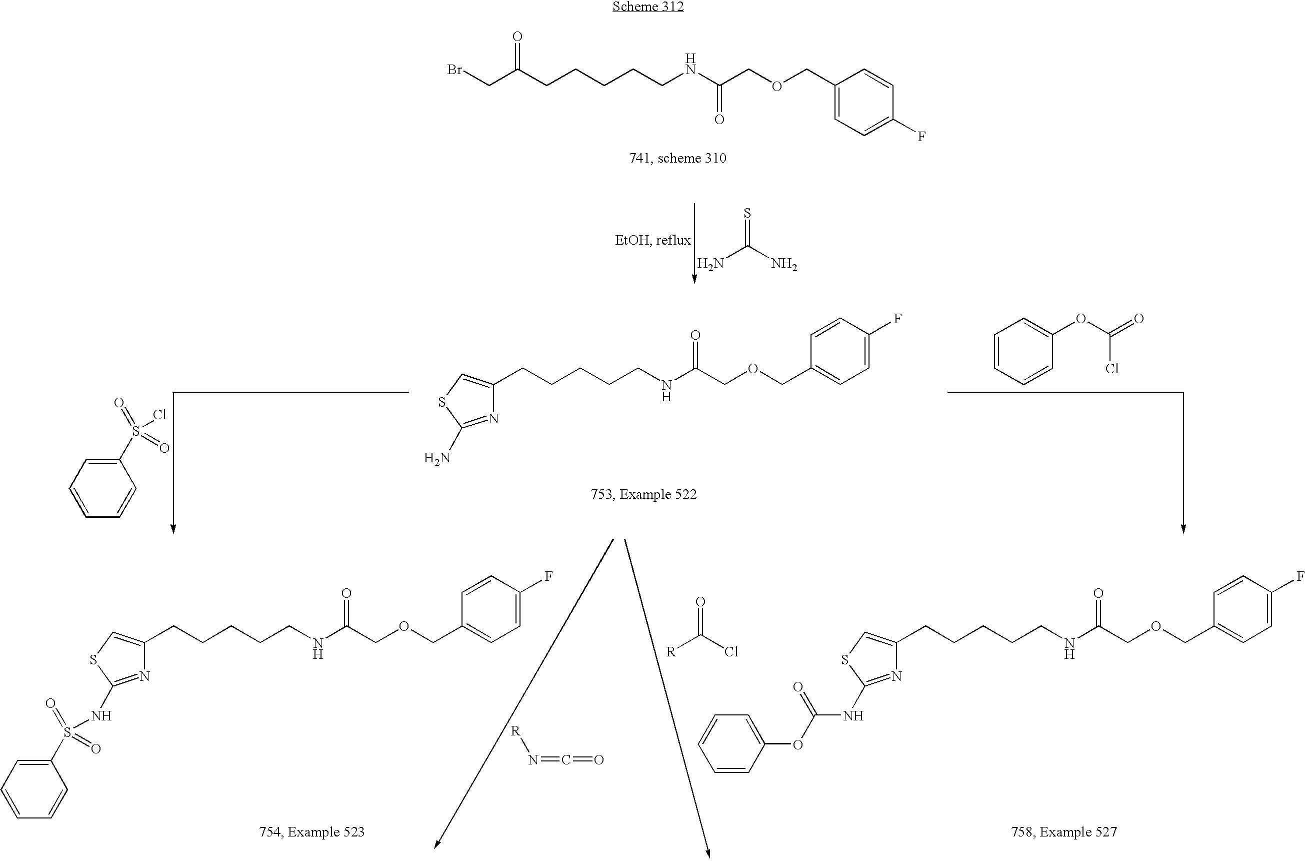

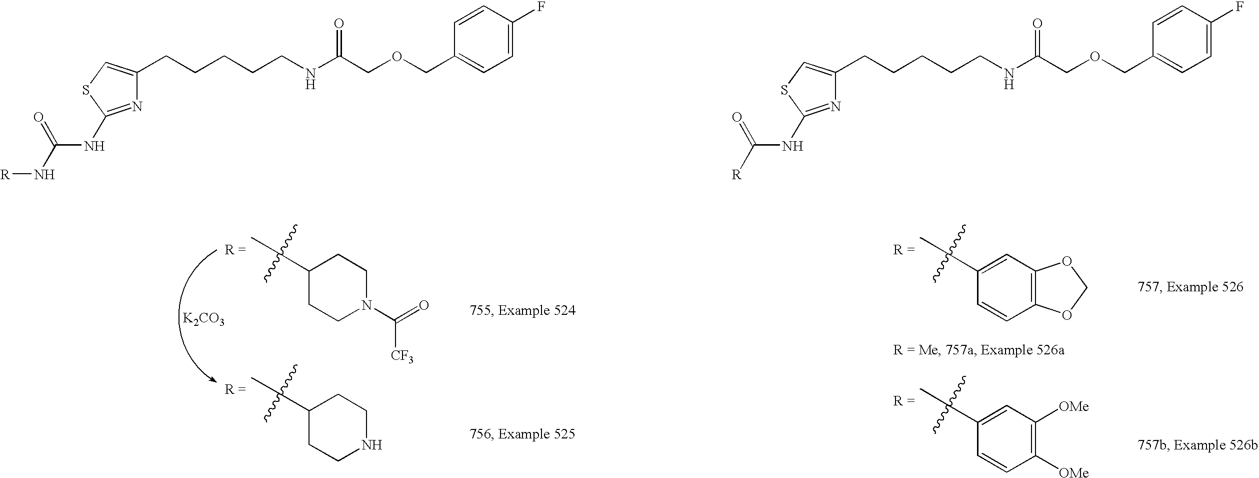

### EXAMPLE 522

### N-(5-(2-aminothiazol-4-yl)pentyl)-2-(4-fluorobenzyloxy)acetamide (753)

A mixture of alpha-bromoketone 741, scheme 310 (90 mg, 0.24), and thiourea (36.6 mg, 0.48 mmol) in EtOH (5 ml) was refluxed for 48 h. The mixture was taken to dryness and the residue was purified by silica gel column chromatography with gradient of EtOAc (30-60%) in Hexanes then with gradient of MeOH (1-10%) in EtOAc. To give compound 753 (83% yield) as clear oil.

(MEOD-d₄) δ (ppm) ¹H 7.39 (m, 2H), 7.07 (m, 2H), 6.08 (s, 1H), 4.55 (s, 2H), 3.92 (s, 2H), 3.22 (t, J=7 Hz, 2H), 2.47, J=0.7, 7 Hz, 2H), 1.62 (m, 2H), 1.52 (m, 2H), 1.34 (m, 2H). LRMS (ESI): (calc.) 351.4, (found) 352.1 (MH)⁺.

### EXAMPLE 523

### 2 (754-)-5-(2-aminothiazol-4-yl)pentyl)acetamide (753)

Aminothiazole 753 (40 mg, 0.124 mmol) and benzene sulfonylchloride (44□L, 0.343 mmol) was dissolved in 0.5 mL of pyridine, and a solution of 4-dimethylaminopyridine (4 mg, 0.43 mmol) in pyridine 90.5 mL) was added and the mixture was shaken for 22 hours. The orange solution was, concentrated, diluted with 1 M Na₂CO₃(aq.) and extracted with DCM. The organic layer was washed with 1 N HCl(aq), dried (anhydrous Na₂SO₄) and concentrated. The crude material was purified by silica gel flash chromatography through a Biotage 12M column using a gradient of methanol (0 to 20%) in DCM. It was then further purified by preparative HPLC (Aquasil C18 20×250 mm, 50/50 to 100/0 methanol/water (0.1% formic acid) in 45 min at 10 mL/min) to give 754 as a white solid after lyophilization (17.2 mg, 31%).

(DMSO-d₆) δ (ppm) ¹H: 12.7 (s, 1H), 7.77-7.53 (m, 3H), 7.55-7.48 (m, 3H), 7.42-7.38 (m 2H), 7.17 (t, J=8.8 Hz, 2H), 6.37 (s, 1H), 4.48 (s, 2H), 3.83 (s, 2H), 3.02 (q, J=6.4 Hz, 2H), 2.36 (t, J=7.2 Hz, 2H), 1.49 (quint, J=7.6 Hz, 2H), 1.38 (quint, J=7.6 Hz, 2H), 1.21-1.17 (m 2H). LRMS (ESI): (calc) 491.1; (found) 492.1 (MH)⁺, 514.0 (MNa)⁺.

### EXAMPLE 524

### 2-(4-fluorobenzyloxy)-N-(5-(2-(3-(1-(2,2,2-trifluoroacetyl)piperidin-4-yl)ureido)thiazol-4-yl)pentyl)acetamide (755)

A solution of the aminothiazole 753 (62.5 mg, 0.178 mmol) and N-(trifluroacetyl)-piperidine-4-isocyanate (79 mg, 0.356 mmol) in 1 mL THF inside a sealed vial was shaken on a mechanical shaker for 5.5 hours at 50° C. The reaction was cooled and PS-trisamine was added. The suspension was shaken for an additional 2.5 hours. It was filtered and the resin washed with CH₂Cl₂. The filtrate was concentrated and purified by silica gel flash chromatography through a Biotage 12M column using a gradient of MeOH (0-15%) in ethyl acetate, the material was further purified by preparative HPLC (Aquasil C18 20×250 mm, methanol (0-50%) in water (0.1% formic acid) in 45 min at 10 mL/min) to give pure 755 (29 mg) as a semi-crystalline solid.

(DMSO-d₆) δ (ppm) ¹H: 10.25 (s,1H), 7.77 (t, J=6.0 Hz, 1H), 7.41-7.38 (m, 2H), 7.19-7.14 (m, 2H), 6.61 (d, J=7.6 Hz, 1H), 6.53 (s, 1H), 4.48 (s, 2H), 4.15-4.10 (m, 1H), 3.84 (s, 2H), 3.79-3.76 (m, 2H), 3.10-3.03 (m, 3H), 1.94-1.89 (m, 2H), 1.56 (quint, J=7.2 Hz, 2H), 1.44-1.37 (m, 4H) 1.26-1.20 (m, 2H). LRMS (ESI): (calc) 573.2; (found) 574.0 (MH)⁺, 596.0 (MNa)⁺

### EXAMPLE 525

### 2-(4-fluorobenzyloxy)-N-(5-(2-(3-piperidin-4-ylureido)thiazol-4-yl)pentyl)acetamide hydrochloride (756)

To a solution of 755, scheme 312 (52 mg, 0.091 mmol) in methanol (1 mL) of and H₂O (0.5 mL) was added potassium carbonate (50 mg, 0.364 mmol). The reaction was stirred at room temperature for 2 h, and then it was concentrated, diluted with water (10 mL) and extracted into EtOAc (3×7 mL). The combined organic extracts were washed with brine, dried (anhydrous Na₂SO₄) and concentrated. Addition of HCl in CH₂Cl₂ to the crude gave the hydrochloride salt of 756 as a semi-crystalline material. The compound was further purified by trituration with methanol and diethyl ether and then with acetonitrile to give 756 as white powder (23 mg 49%).

(MEOD-d₄) δ (ppm) ¹H: 7.42-7.38 (m, 2H), 7.11-7.06 (m, 2H), 6.91 (s, 1H), 4.56 (s, 2H), 3.98-3.95 (m, 1H), 3.92 (s, 2H), 3.43 (ddd, J=3.6, 3.6, 13.6 Hz, 2H), 3.24 (t, J=6.8 Hz, 2H), 3.18-3.11 (m, 2H), 2.71 (t, J=7.6 Hz, 2H), 2.20-2.16 (m, 2H), 1.86-1.76 (m, 2H), 1.72 (quint, J=7.6 Hz, 2H), 1.56 (quint, J=7.26 Hz, 2H), 1.41-1.36 (m, 2H). LRMS (ESI): (calc) 477.2; (found) 478.1 (MH)⁺, 500.0 (MNa)⁺.

### EXAMPLE 526

### N-(4-(5-(2-(4-fluorobenzyloxy)acetamido)pentyl)thiazol-2-yl)benzo[d][1,3]dioxole-5-carboxamide (757)

Piperonyloyl chloride (19.1 mg, 0.1 mmol) was added to amine 753 (24.3 mg, 0.07 mmol) and dimethylaniline (12.6 mg, 0.1 mmol) in THF (1 ml). After 16 at room temperature the reaction was slow so DMAP (cat. amount) was added and immediate reaction was observed and a precipitate formed. Sat'd NaHCO₃, and EtOAc were added and the mixture was stirred for 30 min, then the organic layer was separated and extracted with H₂O, then 1 N HCl, dried (MgSO₄), filtered and concentrated and purified by silica gel column chromatography with gradient of EtOAc (60-80%) in Hexanes to give amide 757 (23.7% yield) as clear oil.

(CDCl₃) δ (ppm) ¹H, 7.52 (d, J=8 Hz, 1H), 7.45 (s, 1H), 7.28 (mk, 2H), 7.04 (m, 2H), 6.87 (dxd, J=8.2, 2.7 Hz, 1H0, 6.61 (bs, 1H), 6.55 (s,1H), 6.06 (s, 2H), 4.52 (s, 2H), 4.0 (s, 2H), 3.27 (m, 2H), 2.58 (m, 2H), 1.67 (m, 2H), 1.52 (m, 2H), 1.33 (m, 2H). LRMS (ESI): (calc.) 499.6; (found) 500.0 (MH)⁺.

### EXAMPLE 526a

### N-(5-(2-acetamidothiazol-4-yl)pentyl)-2-(4-fluorobenzyloxy)acetamide (757a)

Amine 753 (47.5 mg, 0.135 mmol) and 3,4-dimethoxybenzene-1-sulfonyl chloride (32 mg, 0.135 mmol), pyridine (54 uL) and acetic anhydride (0.27 ml) were heated at 100° C. for 16 h according to the procedure of A. S. Gupta et al., (Indian J. Chem. Sec. B, 1996, 967-969). The progress of the reaction was followed by MS. The only signal observed is that of the acetylated amine. The mixture was taken to dryness, EtOAc and sat'd NaHCO₃ were added and the organic layer was washed with H₂O, and 1 N HCl, dried (MgSO₄) filtered, concentrated and purified by silica gel column chromatography with gradient of EtOAc (20-100%) in Hexanes, then with MeOH (3%) in EtOAc. Amide 757a was obtained as clear semi-crystalline solid (56.6% yield).

(CDCl₃) δ (ppm) ¹H: 9.2 (bs, 1H), 7.29 (m, 2H), 7.05 (m, 2H), 6.61 (bs, 1H), 6.5 (s, 1H), 4.52 (s, 2H), 3.98 (s, 2H), 3.28 (m, 2H), 2.53 (m, 2H), 2.21 (s, 3H), 1.65 (m, 2H), 1.55 (m, 2H), 1.37 (m, 2H). LRMS (ESI): (calc.) 393.5; (found) 394.1 (MH)⁺.

### EXAMPLE 526b

### N-(4-(5-(2-(4-fluorobenzyloxy)acetamido)-pentyl)thiazol-2-yl)-3,4-dimethoxybenzamide (757b)

3,4-Dimethoxybenzoylchloride (69 mg, 0.344 mmol) was added to a suspension of aminothiazole 753 (41 mg, 0.117 mmol), 4-dimethy-laminopyridine (4 mg 0.032 mmol), and PS-DIEA (0.468 mmol) in THF (1 mL). The suspension was heated to 50° C. for 2 hours on a mechanical shaker: The reaction was cooled and PS-trisamine (0.468 mmol) was added. The suspension was shaken for a further 2 hours at room temperature, then the resin was filtered, and the filtrate was concentrated and purified by silica gel flash chromatography through a Biotage 12M column using a gradient of EtOAc (50-100%) in CH₂Cl₂ and then a gradient of MeOH (0-15%) in EtOAc to obtain 757b as an oily white solid (25 mg 42%).

(DMSO-d₆) δ(ppm) ¹H: 12.43 (s, 1H), 7.78 (t, J=6.0 Hz, 1H), 7.74-7.71 (m, 2H), 7.41-7.38 (m, 2H), 7.19-7.14 (m, 2H), 7.07 (d, J=8.4 Hz, 1H), 6.78 (s, 1H), 4.48 (s, 2H), 3.84 (s, 2H), 3.83 (s, 3H), 3.82 (s, 3H), 3.08 (q, J=6.8 Hz, 2H), 2.60 (t, J=7.6 Hz, 2H), 1.64 (quint, J=7.6 Hz, 2H), 1.44 (quint, J=7.6 Hz, 2H), 1.28-1.24 (m, 2H). LRMS (ESI): (calc.) 515.2; (found0 516.0 (MH)⁺, 538.0 (MNa)+

### EXAMPLE 527

### Phenyl 4-(5-(2-(4-fluorobenzyloxy)acetamido)-pentyl)thiazol-2-ylcarbamate (758)

To a solution of amine 753 (22 mg, 0.063 mmol) in THF (1 mL) was added PS-DIEA (52 mg, 0.189 mmol) followed by phenylchloroformate (16uL, 0.125 mmol). The suspension was stirred for 3.5 h at room temperature before the addition of PS-trisamine (31 mg, 0.125 mmol) to scavenge the excess phenylchloroformate. The suspension was shaken overnight on a mechanical shaker and then filtered. The resin was washed with DCM and then the filtrate concentrated to give the crude material. The crude was purified by preparative TLC using EtOAc (60%) in DCM to give 758 as a semi-crystalline white solid (15 mg, 49%).

(DMSO-d₆) δ (ppm) ¹H: 12.21 (s, 1H), 7.78 (t, J=5.6 Hz, 1H), 7.44-7.39 (m, 4H), 7.27 (t, J=7.2 Hz, 1H), 7.22-7.15 (m, 4H), 6.76 (s, 1H), 4.48 (s, 2H), 3.85 (s, 2H), 3.07 (q, J=6.4 Hz, 2H), 2.55 (t, J=7.6 Hz, 2H), 1.60 (quint, J=7.2 Hz, 2H), 1.43 (quint, J=7.2 Hz, 2H), 1.30-1.23 (m, 2H). LRMS (ESI): (calc) 471.2; (found) 472.0 (MH)⁺, 494.0 (MNa)⁺.

Examples 523a-d describe the preparation of compounds 754a-d using the same procedures as described for compound 754, Example 523, scheme 312. Characterization data are presented in Table 36

Examples 524a- describe the preparation of compounds 755a- using the same procedure as described for compound 755, Example 524, scheme 312. Characterization data are presented in Table 36.

Example 526c describe the preparation of compound 757c using the same procedure as described for compound 757b, Example 526b, scheme 312. Characterization data are presented in Table 36.

TABLE 36

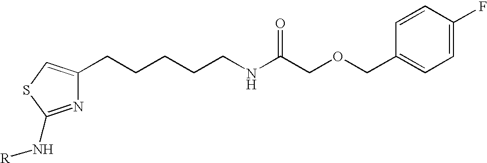

| Ex | Cpd | R | Name | Characterization | Scheme |
|---|---|---|---|---|---|
| 523a | 754a | 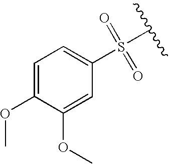 | N-(5-(2-(3,4- dimethoxyphenyl- sulfonamido) thiazol-4-yl) pentyl)-2-(4- fluorobenzyloxy) acetamide | (DMSO-d₆) δ (ppm) ¹H: 7.756 (t, J=6.0Hz, 1H), 7.42-7.38 (m, 2H), 7.34 (dd, J=2.0, 8.4Hz, 1H), 7.22 (d, J=2.0Hz, 1H), 7.19-7.14 (m, 2H), 7.02 (d, J=8.4Hz, 1H), 6.31 (s, 1H), 4.48 (s, 2H), 3.83 (s, 2H), 3.77 (s, 3H), 3.76 (s, 3H), 3.05 (q, J=6.8Hz, 2H), | |
| # 2.48 (t, J=7.6Hz, 2H), 1.49 (quint, J=7.6Hz, 2H), 1.39 (quint, J=7.2Hz, 2H), 1.21-1.17 (m, 2H). LRMS (ESI): (calc) 551.2; (found) 552.1 (MH)⁺, 574.01 (MNa)⁺. | 312 Ex 523 | | | | |
| 523b | 754b | 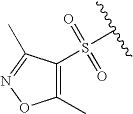 | N-(5-(2-(3,5- dimethylisoxazole- 4-sulfonamido) thiazol-4-yl)pentyl)- 2-(4- fluorobenzyloxy) acetamide | (CDCl₃) δ (ppm) ¹H: 7.32-7.28 (m, 2H), 7.08-7.03 (m, 2H), 6.51 (t, J=6.0Hz, 1H), 6.08 (s, 1H), 4.53 (s, 2H), 4.00 (s, 2H), 3.29 (q, J=6.8Hz, 2H), 2.67 (s, 3H), 2.60 (t, J=7.6Hz, 2H), 2.36 (s, 3H), 1.67 (quint. J=7.6Hz, 2H), 1.55 (quint. J=7.2Hz, 2H), | |
| # 1.37-1.33 (m 2H). LRMS (ESI): calc 510.1; (found) 511.1 (MH)⁺, 533.0 (MNa)⁺. | 312 Ex 523 | | | | |
| 523c | 754c | 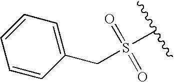 | 2-(4- fluorobenzyloxy)- N-(5-(2- (phenylmethyl- sulfonamido)thiazol- 4-yl)pentyl) acetamide | (DMSO-d₆) δ (ppm) ¹H: 12.4 (s, 1H), 7.72 (t, J=6.0Hz, 1H), 7.37-7.34 (m, 2H), 7.28-7.21 (m, 5H), 7.14-7.10 (m, 2H), 6.22 (s, 1H), 4.44 (s, 2H), 4.20 (s, 2H), 3.02 (q, J=6.0Hz, 2H), 2.33 (t, J=7.6Hz, 2H), 1.47 (quint, J=7.6Hz, 2H), 1.37 (quint, J=7.2Hz, 2H), | |
| # 1.17-1.14 (m 2H). LRMS (ESI): calc 505.2; (found) 506.1 (MH)⁺, 528.1 (MNa)⁺. | 312 Ex 523 | | | | |
| 523d | 754d | 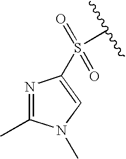 | N-(5-(2-(1,2- dimethyl-1H- imidazole-4- sulfonamido)thiazol- 4-yl)pentyl)-2-(4- fluorobenzyloxy) acetamide | (DMSO-d₆) δ (ppm) ¹H: 7.78 (t, J=6.0Hz, 1H), 7.48 (s, 1H), 7.42-7.38 (m, 2H), 7.19-7.15 (m, 2H), 6.27 (s, 1H), 4.48 (s, 2H), 3.84 (s, 2H), 3.52 (s, 3H), 3.05 (q, J=6.8Hz, 2H), 2.36 (t, J=7.2Hz, 2H), 1.50 (quint, J=7.2Hz, 2H), 1.40 (quint, J=7.6Hz, | |
| # 2H), 1.21-1.17 (m 2H). LRMS (ESI): calc 509.2; (found) 510.22 (MH)⁺, | 312 Ex 523 | | | | |
| 524a | 755a | 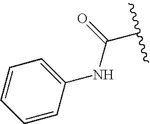 | 2-(4- fluorobenzyloxy)- N-(5-(2-(3- phenylureido) thiazol-4-yl) pentyl)acetamide | (DMSO-d₆) δ (ppm) ¹H: 10.42 (s, 1H), 8.90 (s, 1H), 7.77 (t, J=5.6Hz, 1H), 7.45-7.38 (m, 4H), 7.29 (t, J=7.6Hz, 2H), 7.18-7.14 (m, 2H), 7.00 (t, J=7.2Hz, 1H), 6.62 (s, 1H), 4.48 (s, 2H), 3.84 (s, 2H), 3.07 (q, J=6.8Hz, 2H), 2.52 (t, J=7.2Hz, 2H), 1.59 (quint, J=7.2Hz, | |
| # 2H), 1.43 (quint, J=7.2Hz, 2H), 1.29-1.23 (m, 2H) LRMS (ESI): calc 470.2; (found) 471.1 (MH)⁺, 493.1 (MNa)⁺. | 312 Ex 524 | | | | |
| 524b | 755b | 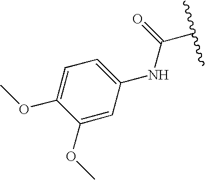 | N-(5-(2-(3-(3,4- dimethoxyphenyl) ureido)thiazol-4- yl)pentyl)-2-(4- fluorobenzyloxy) acetamide | (DMSO-d₆) δ (ppm) ¹H: 10.38 (s, 1H), 8.75 (s, 1H), 7.77 (t, J =6.0Hz, 1H), 7.42-7.38 (m, 2H), 7.19-7.14 (m, 3H), 6.87 (s, 2H), 6.60 (s, 1H), 4.48 (s, 2H), 3.84 (s, 2H), 3.72 (s, 3H), 6.70 (s, 3H), 3.07 (q, J=6.8Hz, 2H), 2.48 (t, J=7.2Hz, 2H), 1.59 (quint, J=7.2Hz, | |
| # 2H), 1.43 (quint, J=7.2Hz, 2H), 1.26-1.23 (m, 2H). LRMS (ESI): calc 530.2; (found) 531.2 (MH)⁺, 553.1 (MNa)⁺. | 312 Ex 524 | | | | |
| 524c | 755c | 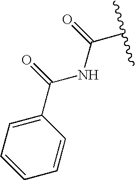 | N-(4-(5-(2-(4- fluorobenzyloxy) acetamido) pentyl)thiazol-2- ylcarbamoyl) benzamide | (DMSO-d₆) δ (ppm) ¹H: 11.86 (s, 1H), 11.46 (s, 1H), 8.00 (d, J=7.6Hz, 2H), 7.79 (t, J=5.6, 1H), 7.66 (t, J=7.2Hz, 1H), 7.54 (t, J=8.0Hz, 2H), 7.42-7.38 (m, 2H), 7.19-7.14 (m, 2H), 6.82 (s, 1H), 4.48 (s, 2H), 3.84 (s, 2H), 3.07 (q, J=6.4Hz, 2H), 2.56 (t, J=7.6Hz, 2H), | |
| # 1.60 (quint, J=7.6Hz, 2H), 1.43 (quint, J=7.6Hz, 2H), 1.29-1.21 (m, 2H) LRMS (ESI): calc 498.2; (found) 499.2 (MH)⁺, 521.2 (MNa)⁺. | 312 Ex 524 | | | | |
| 524d | 755d | 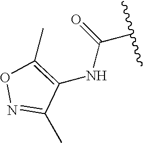 | N-(5-(2-(3-(3,5- dimethylisoxazol-4- yl)ureido)thiazol-4- yl)pentyl)-2-(4- fluorobenzyloxy) acetamide | (DMSO-d₆) δ (ppm) ¹H: 10.80 (s, 1H), 8.06 (s, 1H), 7.78 (t, J=5.6, 1H), 7.42-7.37 (m, 2H), 7.20-7.14 (m, 2H), 6.60 (s, 1H), 4.48 (s, 2H), 3.84 (s, 2H), 3.06 (q, J=6.4Hz, 2H), 2.50 (t, J=8.0Hz, 2H), 2.73 (s, 3H), 2.07 (s, 3H), 1.57 (quint, J=7.2Hz, 2H), | |
| # 1.42 (quint, J=7.2Hz, 2H), 1.25-1.21 (m, 2H) LRMS (ESI): calc 489.2; (found) 490.2 (MH)⁺, | 312 Ex 524 | | | | |
| 524e | 755e | 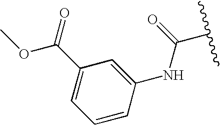 | methyl 3-(3-(4-(5- (2-(4- fluorobenzyloxy) acetamido)pentyl) thiazol-2-yl) ureido)benzoate | (DMSO-d₆) δ (ppm) ¹H: 10.51 (s, 1H), 9.15 (s, 1H), 8.19 (s, 1H), 7.77 (t, J=5.6Hz, 1H), 7.64 (d, J=6.8Hz, 1H), 7.59 (d, J=7.6Hz, 1H), 7.45-7.38 (m, 3H), 7.19-7.14 (m, 2H), 6.63 (s, 1H), 4.48 (s, 2H), 3.84 (s, 2H), 3.07 (q, J=6.4Hz, 2H), 2.52 (t, J=7.2Hz, 2H), 1.59 | |
| # (quint, J=7.6 Hz, 2H), 1.43 (quint, J=6.8Hz, 2H), 1.27-1.23 (m, 2H) LRMS (ESI): calc 528.2; (found) 529.2 (MH)⁺, 551.2 (MNa)⁺. | 312 Ex 524 | | | | |
| 524f | 755f | 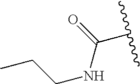 | 2-(4- fluorobenzyloxy)- N-(5-(2-(3- propylureido) thiazol-4-yl) pentyl)acetamide | (CDCl₃) δ (ppm) ¹H: 7.30-7.27 (m, 2H), 7.08-7.02 (m, 2H), 6.59 (t, J=5.6Hz, 1H), 6.31 (s, 1H), 4.51(s, 2H), 3.96 (s, 2H), 3.3-3.25 (m, 4H), 2.60 (t, J=7.6Hz, 2H), 1.66 (quint, J=7.6 Hz, 2H), 1.62-1.52 (m, 4H), 1.37-1.32 (m, 2H), 0.95 (t, J=7.2Hz, 3H). LRMS (ESI): | |
| # calc 436.2; (found) 437.2 (MH)⁺, 459.2 (MNa)⁺. | 312 Ex 524 | | | | |
| 524g | 755g | 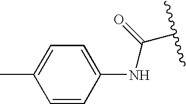 | 2-(4- fluorobenzyloxy)- N-(5-(2-(3-p- tolylureido) thiazol-4-yl) pentyl)acetamide | (DMSO-d₆) δ (ppm) ¹H: 10.40 (s, 1H), 8.82 (s, 1H), 7.78 (t, J=5.6Hz, 1H), 7.42-7.38 (m, 2H), 7.32 (d, J=8.0Hz, 2H), 7.19-7.14 (m, 2H), 7.09 (d, J=8.8Hz, 2H), 6.76 (s, 1H), 4.48 (s, 2H), 3.84 (s, 2H), 3.07 (q, J=7.2Hz, 2H), 2.51 (t, J=7.2Hz, 2H), 2.31 (s, 3H), 1.59 | |
| # (quint, J=7.6Hz, 2H), 1.43 (quint, J=7.2Hz, 2H), 1.30-1.23 (m, 2H) LRMS (ESI): calc 484.2; (found) 485.0 (MH)⁺, 507.0 (MNa)⁺. | 312 Ex 524 | | | | |
| 524h | 755h | 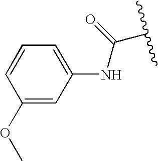 | 2-(4- fluorobenzyboxy)- N-(5-(2-(3-(3- methoxyphenyl) ureido)thiazol-4- yl)pentyl)acetamide | (DMSO-d₆) δ (ppm) ¹H: 10.41 (s, 1H), 8.92 (s, 1H), 7.78 (t, J=5.6Hz, 1H), 7.42-7.38 (m, 2H), 7.20-7.14 (m, 4H), 6.93 (d, J=8.0Hz, 1H), 6.62 (s, 1H), 6.58 (dd, J=2.4, 8.4Hz, 1H), 4.48 (s, 2H), 3.84 (s, 2H), 3.72 (s, 3H), 3.07 (q, J=6.0Hz, 2H), 2.52 (t, J=7.2Hz, | |
| # 2H), 1.59 (quint, J=7.6Hz, 2H), 1.43 (quint, J=7.2Hz, 2H), 1.30-1.23 (m, 2H) LRMS (ESI): calc 500.2; (found) 501.0 (MH)⁺, 523.0 (MNa)⁺. | 312 Ex 524 | | | | |
| 524i | 755i | 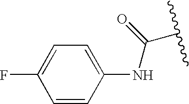 | 2-(4- fluorobenzyloxy)- N-(5-(2-(3-(4- fluorophenyl) ureido)thiazol-4- yl)pentyl)acetamide | (DMSO-d₆) δ (ppm) ¹H: 10.43 (s, 1H), 8.95 (s, 1H), 7.78 (t, J=6.0Hz, 1H), 7.48-7.44 (m, 2H), 7.42-7.38 (m, 2H), 7.19-7.11 (m, 4H), 6.62 (s, 1H), 4.48 (s, 2H), 3.84 (s, 2H), 3.07 (q, J=6.0Hz, 2H), 2.52 (t, J=7.2Hz, 2H), 1.59 (quint, J=7.6Hz, 2H), 1.43 (quint, | |
| # J=7.2Hz, 2H), 1.28-1.23 (m, 2H) LRMS (ESI): calc 488.2; (found) 489.0 (MH)⁺, 511.0 (MNa)⁺. | 312 Ex 524 | | | | |
| 524j | 755j | 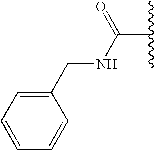 | N-(5-(2-(3- benzylureido) thiazol-4-yl) pentyl)-2-(4- fluorobenzyloxy) acetamide | (CD₃OD) δ (ppm) ¹H: 7.41-7.37 (m, 2H), 7.32-7.30 (m, 4H), 7.26-7.22 (m, 1H), 7.07-7.05 (m, 2H), 6.51 (s, 1H), 4.54 (s, 2H), 4.42 (s, 2H), 3.91 (s, 2H), 3.21 (t, J=7.2Hz, 2H), 2.58 (t, J=7.2Hz, 2H), 1.54 (quint, J=8.0Hz, 2H), 1.52 (quint, J=7.2Hz, 2H), | |
| # 1.36-1.30 (m, 2H) LRMS (ESI): calc 484.2; (found) 485.1 (MH)⁺, 507.0 (MNa)⁺. | 312 Ex 524 | | | | |
| 526c | 757c | 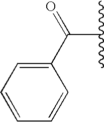 | N-(4-(5-(2-(4- fluorobenzyloxy) acetamido)pentyl) thiazol-2- yl)benzamide | (DMSO-d₆) δ (ppm) ¹H: 12.43 (s, 1H), 7.78 (t, J=6.0Hz, 1H), 7.74-7.71 (m, 2H), 7.41-7.38 (m, 2H), 7.19-7.14 (m, 2H), 7.07 (d, J=8.4Hz, 1H), 6.78 (s, 1H), 4.48 (s, 2H), 3.84 (s, 2H), 3.83 (s, 3H), 3.82 (s, 3H), 3.08 (q, J=6.8Hz, 2H), 2.60 (t, J=7.6Hz, 2H), 1.64 (quint, J=7.6Hz, | |
| # 2H), 1.44 (quint, J=7.6Hz, 2H), 1.28-1.24 (m, 2H) LRMS (ESI): calc 515.2; (found) 516.0 (MH)⁺, 538.0 (MNa)⁺. | 312 Ex 526b | | | | |

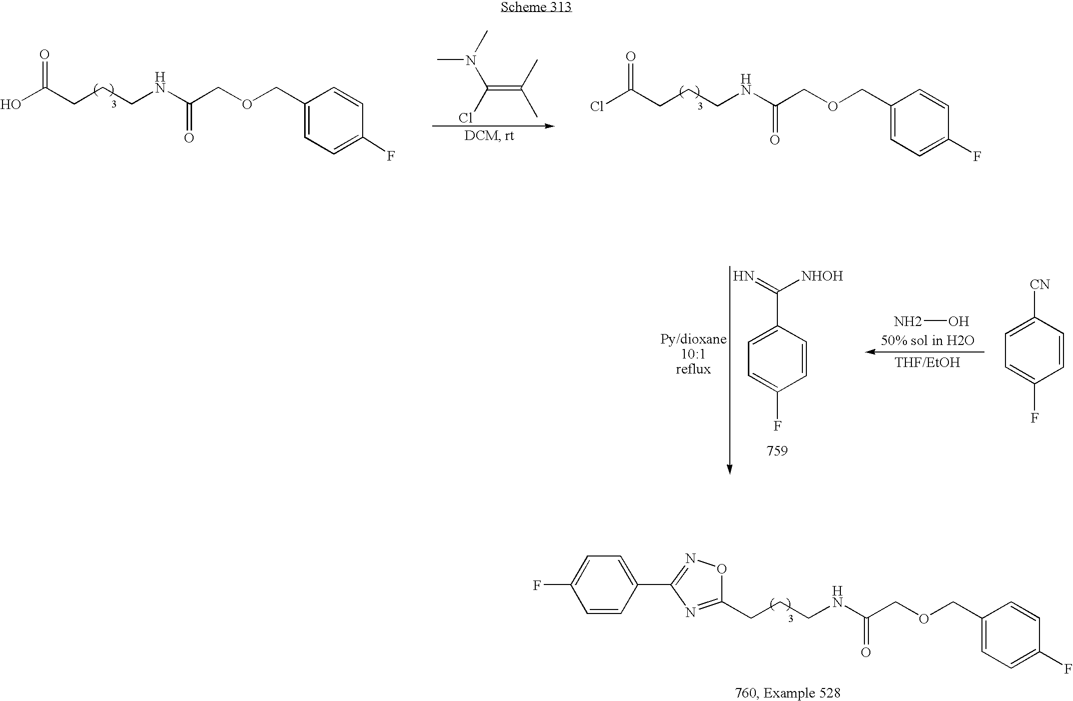

### EXAMPLE 528

### 2-(4-fluorobenzyloxy)-N-(5-(3-(4-fluorophenyl)-1,2,4-oxadiazol-5-yl)pentyl)acetamide (760)

### Step 1: 4-fluoro-N-hydroxybenzimidamide (759)

Hydroxylamine (0.35 ml, 50% solution in H₂O, 5.7 mmol) was added to a solution of 4-fluorobenzonitrile (0.59 g, 4.87 mmol) in THF (5 ml) and EtOH (1 ml) and the mixture was stirred at room temperature for 1 h. The reaction mixture was concentrated and triturated from hexanes leaving 759 as a white solid (0.598 g, 79.6% yield). (DMSO-d6₃) δ (ppm) ¹H: 9.63 (s, 1H), 7.68 (m, 2H), 7.19 (m, 2H), 5.84 (bs, 2H). LRMS (ESI): (calc.) 154.1; (found) 155.1 (MH)⁺.

### Step 2: 2-(4-fluorobenzyloxy)-N-(5-(3-(4-fluorophenyl)-1,2,4-oxadiazol-5-yl)pentyl)acetamide (760)

1-chloro-N,N,2-trimethylprop-1-en-1-amine (133.6 mg/140 uL, 1 mmol) was added to a solution of 6-(2-(4-fluorobenzyloxy)acetamido)hexanoic acid (230 g, 0.77 mmol) in DCM (2 ml) at room temperature under N₂. After 3 h, amidine 759 (118.7 mg, 0.77 mmol), pyridine (1 ml) and dioxane (10 ml) were addded and the mixture was refluxed for 16 h. The mixture was cooled to room temperature and H₂O was added (20 ml) and the oil that formed was extracted with EtOAc and the organic layer was separated, dried (MgSO₄), filtered, concentrated and purified by silica gel column chromatography with gradient of EtOAc (30-100%) in Hexanes. Compound 760 was obtained as a clear semi-crystalline solid (78% yield).

(CDCl₃) δ (ppm) ¹H: 8.06 (m, 2H), 7.28 (m, 2H), 7.16(m, 2H), 7.05 (m, 2H), 6.57 (bs, 1H), 4.51 (s, 2H), 3.96 (s, 2H), 3.31 (q, J=6.8 Hz, 2H), 2.94 (t, J=7.5, 2H), 1.90 (m, 2H), 1.61 (m, 2H), 1.46 (m, 2H). LRMS (ESI): (calc.) 415.4; (found) 416.1 (MH)⁺.

Following the procedure in scheme 313, example 528, step 1 the following intermediates: 4-fluoro-N-hydroxy-3-methoxybenzimidamide 759a, N-hydroxy-3,4,5-trimethoxybenzimidamide 759b, benzyl 2-(hydroxyamino)-2-iminoethylcarbamate 759c, and 4-(dimethylamino)-N-hydroxybenzimidamide 759d, were obtained in 78%, 73%, 94% and 29% respectively, replacing 4-fluorobenzonitrile with 4-fluoro-3-methoxybenzonitrile, 3,4,5-trimethoxybenzonitrile, benzyl cyanomethylcarbamate, and 4-(dimethylamino)benzonitrile in the order indicated.

Examples 528a-d describe the preparation of compound 760a-d using the same procedure as described for Example 528. Characterization data are presented in Table 37.

TABLE 37

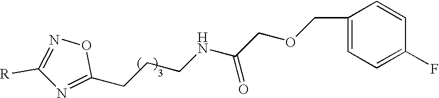

| Ex | Cpd | R₃ | Name | Characterization | Scheme |
|---|---|---|---|---|---|
| 528a | 760a | 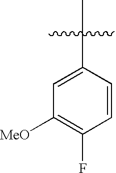 | N-(5-(3-(4-fluoro-3- methoxyphenyl)- 1,2,4-oxadiazol-5- yl)pentyl)-2-(4- fluorobenzyloxy) acetamide | (CDCl₃) δ (ppm) ¹H: 7.65 (m, 2H), 7.28 (m, 2H), dxd, J=Hz, 1H), 7.06 (m, 2H), 6.57 (bs, 1H), 4.51 (s, 2H), 3.96 (s, 3H), 3.95 (s, 2H), 3.31 (q, J=6.9 Hz, 2H), 2.4 (t, J=7.5, 2H), 1.9 (quint, J=7.6 Hz, 2H), 1.6 (m, 2H), 1.45 (m, 2H). LRMS (ESI): (calc.) 445.5; (found) | |
| # 446.1 (MH)⁺ | 313 Int. 759a Ex 528 | | | | |
| 528b | 760b | 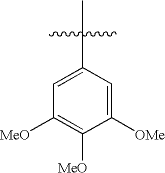 | 2-(4- fluorobenzyloxy)- N-(5-(3-(3,4,5- trimethoxyphenyl)- 1,2,4-oxadiazol-5- yl)pentyl)acetamide | (CDCl₃) δ (ppm) ¹H: 7.31 (s, 2H), 7.28 (m, 2H), 7.05 (m, 2H), 6.58 (bs, 1H), 4.51 (s, 2H), 3.96 (s, 2H), 3.93 (s, 6H), 3.9 (s, 3H), 3.31 (q, J=7 Hz, 2H), 2.95 (t, J=7.6 Hz, 2H), 1.91 (quint, J=7.6 Hz, 2H), 1.58 (m, 2H), 1.45 (m, 2H) LRMS (ESI): (calc.) 487.5; (found) | |
| # 488.1 (MH)⁺ | 313 Int. 759b Ex 528 | | | | |
| 528c | 760c | 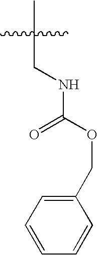 | benzyl (5-(5-(2-(4- fluorobenzyloxy) acetamido)pentyl)- 1,2,4-oxadiazol-3- yl)methylcarbamate | (CDCl₃) δ (ppm) ¹H: 7.36-7.25 (m, 7H), 7.06 (m, 2H), 6.56 (bs, 1H), 5.37 (bs, 1H), 5.14 (s, 2H), 4.5 (overlapped d and s, 4H), 3.95 (s, 2H), 3.28 (q, J=6.8 Hz, 2H), 2.87 (t, J=7.3 Hz, 2H), 1.82 (m, 2H), 1.55 (m, 2H), 1.41 (m, 2H). LRMS (ESI): (calc.) 484.5; (found) 485.1 | |
| # (MH)⁺ | 313 Int. 759c Ex 528 | | | | |
| 528d | 760d | 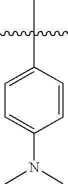 | N-(5-(3-(4- (dimethylamino) phenyl)-1,2,4- oxadiazol-5- yl)pentyl)-2-(4- fluorobenzyloxy) acetamide | (CDCl₃) δ (ppm) ¹H: 7.93 (d, J=9 Hz, 2H), 7.28 (m, 2H), 7.05 (m, 2H), 6.78 (poorly resolved doublet, 2H), 6.57 (poorly resolved triplet, 1H), 4.51 (s, 2H), 3.95 (s, 2H), 3.30 (q, J=6.7 Hz, 2H), 3.04 (s, 6H), 2.92 (t, J=7.4H, 2H), 1.89 (m, 2H), 1.58 (m, 2H), 1.45 | |
| # (m, 2H). LRMS ESI: calc. 440.5; found 441.3 (MH)⁺ | 313 Int. 759d Ex 528 | | | | |

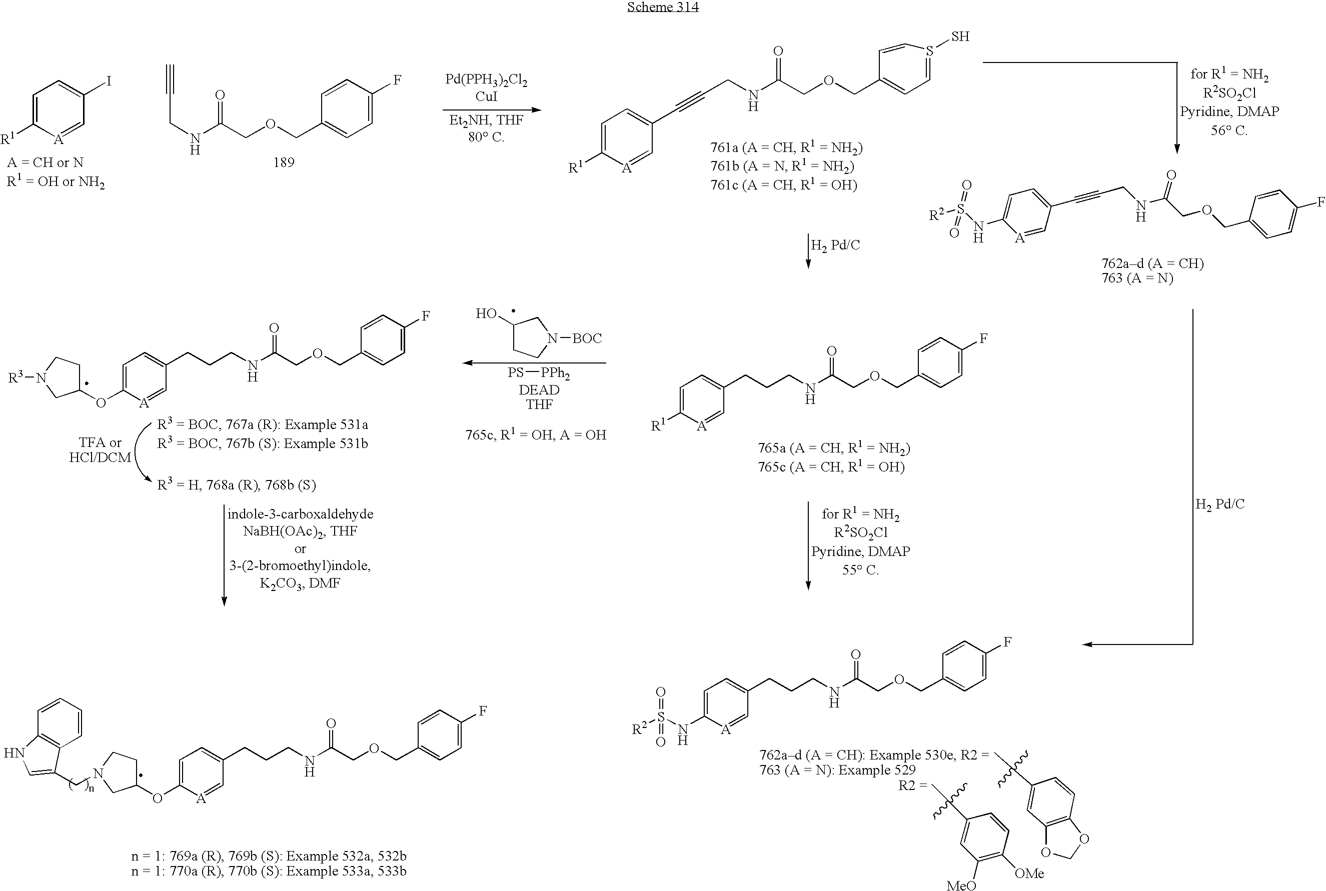

### EXAMPLE 529

### N-(3-(6-(3,4-dimethoxyphenylsulfonamido)pyridin-3-yl)propyl)-2-(4-fluorobenzyloxy)acetamide (764)

### Step 1: N-(3-(6-aminopyridin-3-yl)prop-2-ynyl)-2-(4-fluoro-benzyloxy)acetamide (761b)

A solution of alkyne 189 (100 mg, 0.452 mmol), 2-amino-5-iodopyridine (99.5 mg, 0.452 mmol), Pd(PPh₃)₂Cl₂ (16 mg, 0.023 mmol), and copper iodide (9 mg, 0.45 mmol) in diethylamine (0.23 mL, 2.3 mmol) and THF (2 mL) was stirred at room temperature under nitrogen for 1 hour. An additional amount of 2-amino-5-iodopyridine (10 mg, 0.045 mmol) was tadded and the reaction stirred for another 30 min. The reaction was concentrated and purified by silica gel flash chromatography using a Biotage 12M column and a stepwise gradient of EtOAc (50-100%) in hexanes, then a gradient of MeOH (0-10%) in EtOAc to give 761b as yellow solid (117 mg, 83%).

(DMSO-d₆) δ (ppm) ¹H: 8.31 (t, J=5.6 Hz, 1H), 7.94 (d, J=1.6 Hz, 1H), 7.44-7.40 (m, 2H), 7.34 (dd, J=2.4, 8.8 Hz, 1H), 7.19-7.13 (m, 2H), 6.36 (d, J=8.8 Hz, 1H), 6.31 (s, 2H), 4.50 (s, 2H), 4.10-4.08 (m, 2H), 3.92 (s, 2H).

LRMS (ESI): (calc.) 313.3; (found) 314.0 (MH)⁺.

### Step 2: N-(3-(6-(3,4-dimethoxyphenyl-sulfonamido)pyridin-3-yl)prop-2-ynyl)-2-(4-fluorobenzyloxy)acetamide (763)

Amine 761b (134 mg, 0.428 mmol), 3,4-dimethoxybenzenesulfonyl chloride (152 mg, 0.642 mmol) and 4-dimethyl-aminoaniline (16 mg, 0.128 mmol) were heated at 60° C. in pyridine (2 mL) inside a sealed vial for 24 hours as described for Example 523, scheme 312. Purification by silica gel flash chromatography through a Biotage 25M column using a gradient of EtOAc (50-100%) in DCM gave 763 as a white fluffy solid (53 mg, 24%).

(MEOD-d₄) δ (ppm) ¹H, 8.16 (d, J=2.4 Hz, 1H), 7.34 (dd, J=2.0, 8.4 Hz, 1H), 7.52 (dd, J=2.4, 8.4 Hz, 1H), 7.45 (s, 1H), 7.41-7.38 (m, 2H), 7.12 (d, J=8.4 Hz, 1H), 7.08-7.04 (m, 2H), 7.00 (d, J=8.4 Hz, 1H), 4.56 (s, 2H), 4.20 (s, 2H), 3.96 (s, 2H), 3.84 (s, 3H), 3.82 (s, 3H). LRMS (ESI): (calc.) 513.14; (found) 514.0 (MH)⁺, 536.0 (MNa)⁺.

### Step 3: N-(3-(6-(3,4-dimethoxyphenylsulfonamido)pyridin-3-yl)propyl)-2-(4-fluorobenzyloxy)acetamide (764)

Compound 763 (50 mg, 0.097 mmol) in MeOH (0.3 mL), N,N-dimethylacetamide (0.3 mL) and palladium on activated charcoal (5% wt, 30 mg) was hydrogenated under an 1 atmosphere of hydrogen gas. The material was purified by silica gel flash chromatography using a Biotage 12S column using a gradient of EtOAc (20-100%) in DCM followed by a gradient of MeOH (0-15%) in EtOAc. Trituration with methanol and pentane gave 764 as an off-white powder (21 mg, 42%).

(MEOD-d₄) δ (ppm) ¹H: 7.87 (d, J=1.6 Hz, 1H), 7.57 (dd, J=2.4, 8.8 Hz, 1H), 7.50 (dd, J=2.0, 8.0 Hz, 1H), 7.24 (d, J=2.0 Hz, 1H), 7.41-7.37 (m, 2H), 7.16 (dd, J=0.8, 8.8 Hz, 1H), 7.10-7.05 (m, 2H), 7.00 (d, J=8.4 Hz, 1H), 4.53 (s, 2H), 3.88 (s, 2H), 3.84 (s, 3H), 3.82 (s, 3H), 3.22 (t, J=6.8 Hz, 2H), 2.52 (t, J=7.6 Hz, 2H), 1.76 (quint. J=7.6 Hz, 2H).

LRMS (ESI): (calc.) 517.17; (found) 518.0 (MH)⁺, 540.0 (MNa)⁺.

### EXAMPLE 530e

N-(3-(4-(benzo[d][1,3]dioxole-5-sulfonamido)phenyl)propyl)-2-(4-fluorobenzyloxy)-acetamide (766e)

### Step 1: N-(3-(4-aminophenyl)prop-2-ynyl)-2-(4-fluorobenzyloxy)acetamide (761a)

A solution of alkyne 189 (1.00 g, 4.52 mmol), 4-iodoaniline (0.991 g, 4.52 mmol), Pd(PPh₃)₂Cl₂ (0.159 g, 0.226 mmol), and copper iodide (86 mg, 0.452 mmol) in diethylamine (2.3 mL, 22.6 mmol) and THF (20 mL) was reacted as described for 761b, Example 529, step 1. After 1 h, the mixture was concentrated and purified by silica gel flash chromatography using a stepwise gradient of EtOAc (20-80%) in hexanes to afford 761a as an orange oil (1.13 g, 80%). ¹H NMR (DMSO-d₆) δ (ppm): 8.28 (t, J=5.6 Hz, 1H), 7.44-7.40 (m, 2H), 7.19-7.13 (m, 2H), 7.02 (d, J=8.8 Hz, 2H), 6.46 (d, J=8.8 Hz, 2H); 5.44 (s, 2H), 4.50 (s, 2H), 4.07 (d, J=5.6 Hz, 2H) 3.91 (s, 2H).

### Step 2: N-(3-(4-aminophenyl)propyl)-2-(4-fluorobenzyloxy)acetamide (765a)

Compound 761a (0.7219, 2.31 mmol) in methanol (8 mL) and palladium on charcoal (5% wt, 150 mg) was hydrogenated under an atmosphere of hydrogen gas as described for 763, Example 529, step 3. Purification by chromatography using a stepwise gradient of EtOAc (40-100%) in hexanes gave 765a as pinkish solid (0.470 g, 64%). ¹H NMR (DMSO-d₆) δ (ppm) ¹H: 7.87 (t, J=6.0 Hz, 1H), 7.42-7.39 (m, 2H), 7.19-7.14 (m, 2H), 6.80 (d, J=8.0 Hz, 2H), 6.44(d, J=8.02H), 4.802H), 4.48(s, 2H), 3.85 (s, 2H), 3.06 (q, J=6.8 Hz, 2H), 2.51 (t, J=8.0 Hz, 2H), 1.61 (quint., J=7.6 Hz, 2H). LRMS (ESI): (calc.) 316.4; (found) 317.1 (MH)⁺, 339.0 (MNa)⁺.

### Step 3: N-(3-(4-aminophenyl)propyl)-2-(4-fluorobenzyloxy)acetamide (766e)

Reaction of 765a (80 mg, 0.253 mmol) with 1,3-benzodioxole-5-sulfonylchloride (61.4 mg, 0.278 mmol) and 4-dimethylaminoaniline (9 mg, 0.076 mmol) at 55° C. in pyridine (1 mL) inside a sealed vial for 6 hours as described for compound 754, Example 523, scheme 312. Purification by silica gel flash chromatography using a Biotage 12M column and a gradient of EtOAc (20-90%) in DCM gave 766e as a white solid (86 mg, 68%). (DMSO-d₆) δ (ppm): 10.0 (s, 2H), 7.81 (t, J=5.6 Hz, 1H), 7.415-7.38 (m, 2H), 7.23 (dd, J=1.6, 8.0 Hz, 1H), 7.18-7.14 (m, 3H), 7.04-6.94 (m, 5H), 6.10 (s, 2H), 4.48 (s, 2H), 3.84 (s, 2H), 3.04 (q, J=6.4 Hz, 2H), 2.42 (t, J=8.0 Hz, 2H), 1.62 (quint., J=7.2 Hz, 2H).

LRMS (ESI): (calc.) 500.14; (found) 501.0 (MH)⁺, 523.0 (MNa⁺).

Examples 530a-k describe the preparation of compound 766a-k using the same procedure as described either for for Example 529 or 530e, scheme 314. Characterization data are presented in Table 38.

TABLE 38

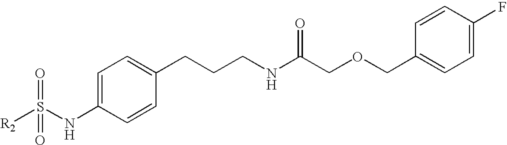

| Ex | No. | R² | Name | Characterization | Scheme |
|---|---|---|---|---|---|
| 530a | 766a | 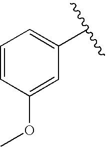 | 2-(4- fluorobenzyloxy)- N-(3-(4-(3- methoxyphenyl- sulfonamido) phenyl)propyl) acetamide | (DMSO-d₆) δ (ppm) ¹H: 6.60-6.56 (m, 2H), 6.53 (d, J=8.4Hz, 1H), 6.47 (m, 1H), 6.39-6.37 (m, 1H), 6.29-6.23 (m, 5H), 6.17 (d, J=8.8Hz, 2H), 3.74 (s, 2H), 3.09 (s, 2H), 2.93 (s, 3H), 2.39 (t, J=7.2Hz, 2H), 1.73 (t, J=7.6Hz, 2H), 0.946 (quint, J=7.2Hz, 2H). LRMS (ESI): | |
| # (calc) 486.2; (found) 487.0 (MH)⁺, 509.0 (MNa)⁺. | 314 Ex 529, step 1-3 | | | | |
| 530b | 766b | 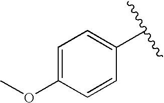 | 2-(4- fluorobenzyloxy)- N-(3-(4-(4- methoxyphenyl- sulfonamido) phenyl)propyl) acetamide | (DMSO-d₆) δ (ppm) ¹H: 9.99 (s, 1H), 7.81 (t, J=6.0Hz, 1H), 7.62 (d, J=8.8Hz, 2H), 7.41-7.38 (m, 2H), 7.18-7.14 (m, 2H), 7.02 (m, 4H), 6.95 (d, J=8.8Hz, 2H), 4.47 (s, 2H), 3.83 (s, 2H), 3.76 (s, 3H), 3.03 (q, J=6.8Hz, 2H), 2.42 (t, J=7.2Hz, 2H), 1.61 (quint. J=7.6Hz, | |
| # 2H). LRMS (ESI): calc 486.2; (found) 487.0 (MH)⁺, 509.0 (MNa)⁺. | 314 Ex 529, step 1-3 | | | | |
| 530c | 766c | 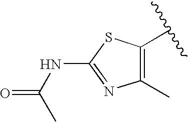 | N-(3-(4-(2- acetamido-4- methylthiazole-5- sulfonamido) phenyl)propyl)-2-(4- fluorobenzyloxy) acetamide | (MEOD-d₄) δ (ppm) ¹H: 7.42-7.38 (m, 2H), 7.11-7.02 (m, 6H), 4.55 (s, 2H), 3.61 (s, 2H), 3.20 (t, J=7.2Hz, 2H), 2.56 (t, J=7.2 Hz, 2H), 2.34 (s, 3H), 2.17 (s, 3H), 1.77 (quint. J=7.2Hz, 2H). LRMS (ESI): calc 534.1; (found) 535.0 (MH)⁺, | |
| # 557.0 (MNa)⁺. | 314 Ex 529, step 1-3 | | | | |
| 530d | 766d | 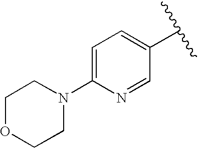 | 2-(4- fluorobenzyloxy)- N-(3-(4-(6- morpholinopyridine- 3-sulfonamido) phenyl)propyl) acetamide | (DMSO-d₆) δ (ppm) ¹H: 9.98 (s, 1H), 8.24 (d, J=2.4Hz, 1H), 7.82 (t, J=6.0Hz, 1H), 7.68 (dd, J=2.4, 9.2Hz, 1H), 7.42-7.38 (m, 2H), 7.18-7.14 (m, 2H), 7.04 (d, J=8.8Hz, 2H), 6.97 (d, J=8.4Hz, 2H), 6.85 (d, J=9.2Hz, 1H), 4.48 (s, 2H), 3.84 (s, 2H), | |
| # 3.63-3.60 (m, 4H), 3.54-3.51 (m, 4H), 3.04 (t, J=6.4Hz, 2H), 2.43 (t, J=6.4Hz, 2H), 1.62 (quint. J=7.2Hz, 2H). LRMS (ESI): calc 542.2; (found) 543.1 (MH)⁺, 565.0 (MNa)⁺. | 314 Ex 529, step 1-3 | | | | |
| 530f | 766f | 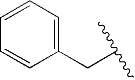 | 2-(4- fluorobenzyloxy)- N-(3-(4- (phenylmethyl- sulfonamido) phenyl)propyl) acetamide | (MEOD-d₄) δ (ppm) ¹H: 7.14-7.38 (m, 2H), 7.31-7.28 (m, 3H), 7.27-7.29 (m, 2H), 7.15-7.05 (m, 6H), 4.55 (s, 2H), 4.33 (s, 2H), 3.91 (s, 2H), 3.25 (t, J=6.8Hz, 2H), 2.56 (t, J=7.2Hz, 2H), 1.81 (quint. J=7.2Hz, 2H). LRMS (ESI): calc 470.2; (found) 471.0 (MH⁺), | |
| # 493.0(MNa)⁺. | 314 Ex 530e, step 1-3 | | | | |
| 530g | 766g | 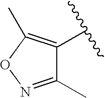 | N-(3-(4-(3,5- dimethylisoxazole- 4-sulfonamido) phenyl)propyl)-2- (4-fluorobenzyloxy) acetamide | (MEOD-d₄) δ (ppm) ¹H: 7.23-7.39 (m, 2H), 7.14 (d, J=8.8Hz, 2H), 7.11-7.06 (m, 2H), 7.02 (d, J=8.4Hz, 2H), 4.57 (s, 2H), 3.92 (s, 2H), 3.23 (t, J=7.2Hz, 2H), 2.59 (t, J=8.0Hz, 2H), 2.36 (m, 3H), 2.19(S, 3H), 1.79 (quint, J=7.6Hz, 2H) LRMS (ESI): calc 475.2; | |
| # (found) 476.0 (MH)⁺, 498.0 (MNa)⁺. | 314 Ex 530e, step 1-3 | | | | |
| 530h | 766h | 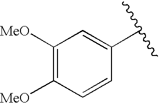 | N-(3-(4-(3,4- dimethoxyphenyl- sulfonamido) phenyl)propyl)-2-(4- fluorobenzyloxy) acetamide | (DMSO-d₆) δ (ppm) ¹H: 9.92 (s, 1H), 7.81 (t, J=6.0Hz, 1H), 7.41-7.38 (m, 2H), 7.26 (dd, J=2.4, 8.4Hz, 1H), 7.18-7.14 (m, 3H), 7.04-7.01 (m, 3H), 6.96 (d, J=8.4Hz, 2H), 4.47 (s, 2H), 3.84 (s, 2H), 3.75 (s, 3H), 3.70 (s, 3H), 3.04 (q, J=6.4Hz, 2H), 2.42 (t, J=7.6Hz, | |
| # 2H), 1.61 (quint, J=7.2Hz, 2H). LRMS (ESI): calc 516.2; (found) 517.0 (MH)⁺, 539.0 (MNa)⁺. | 314 Ex 530e, step 1-3 | | | | |
| 530i | 766i | 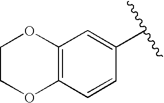 | N-(3-(4-(2,3- dihydrobenzo[b][1,4]dioxine-6- sulfonamido) phenyl)propyl)-2-(4- fluorobenzyloxy) acetamide | (DMSO-d₆) δ (ppm) ¹H: 10.03 (s, 1H), 7.82 (t, J=6.0Hz, 1H), 7.42-7.38 (m, 2H), 7.18-7.14 (m, 4H), 7.03 (d, J=8.8Hz, 2H), 6.98-6.94 (m, 3H), 4.47 (s, 2H), 4.26-4.23 (m, 4H), 3.84 (s, 2H), 3.04 (q, J=6.4Hz, 2H), 2.424 (t, J=7.6Hz, 2H), | |
| # 1.62 (quint, J=7.2Hz, 2H). LRMS (ESI): calc 514.2; obs 515.0 (MH)⁺, 537.0 (MNa)⁺. | 314 Ex 530e, step 1-3 | | | | |
| 530j | 766j | 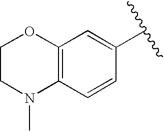 | 2-(4- fluorobenzyloxy)- N-(3-(4-(4- methyl-3,4- dihydro-2H- benzo[b][1,4]oxa zine-7-sulfonamido) phenyl)propyl) acetamide | (DMSO-d₆) δ (ppm) ¹H: 9.90 (s, 1H), 7.82 (t, J=6.0Hz, 1H), 7.42-7.38 (m, 2H), 7.18-7.14 (m, 2H), 7.03 (d, J=8.4Hz, 2H), 6.97 (d, J=8.8Hz, 2H), 6.93-6.90 (m, 2H), 6.70 (d, J=8.8Hz, 1H), 4.48 (s, 2H), 4.23-4.21 (m, 2H), | |
| # 3.84 (s, 2H), 3.23-3.21 (m, 2H), 3.04 (q, J=7.2Hz, 2H), 2.76 (s, 3H), 2.42 (t, J=7.6Hz, 2H), 1.62 (quint, J=7.6Hz, 2H). LRMS (ESI): calc 527.2; (found) 528.0 (MH)⁺, 550.0 (MNa)⁺. | 314 Ex 530e, step 1-3 | | | | |
| 530k | 766k | 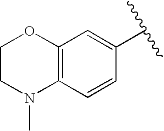 | 2-(4- fluorobenzyloxy)- N-(3-(4- (phenylsulfonamido) phenyl)propyl) acetamide | (DMSO-d₆) δ (ppm) ¹H: 10.15 (s, 1H), 7.81 (t, J=6.0Hz, 1H), 7.72-7.69 (m, 2H), 7.59 7.55 (m, 1H), 7.53-7.49 (m, 2H), 7.41-7.38 (m, 2H), 7.18-7.14 (m, 2H), 7.02 (d, J=8.4Hz, 2H), 6.95 (d, J=8.4Hz, 2H), 4.47 (s, 2H), 3.83 (s, 2H), 3.03 (q, J=6.4Hz, 2H), 2.41 (t, J=7.6Hz, | |
| # 2H), 1.61 (quint, J=7.6Hz, 2H). LRMS (ESI): (calc) 456.2; (found) 457.0 (MH)⁺, 479.0 (MNa)⁺. | 314 Ex 530e, step 1-3 | | | | |

### EXAMPLE 531a

### (R)-tert-butyl 3-(4-(3-(2-(4-fluorobenzyloxy)acetamido)propyl)phenoxy)pyrrolidine-1-carboxylate (767a)

### Step 1: 2-(4-fluorobenzyloxy)-N-(3-(4-hydroxyphenyl)prop-2-ynyl)acetamide (761c)

A solution of alkyne 189 (2.01 g, 9.10 mmol), 4-iodophenol (2.00 g, 9.10 mmol), Pd(PPh₃)₂Cl₂ (0.319 g, 0.455 mmol), and copper iodide (173 mg, 0.910 mmol) in diethylamine (4.7 mL, 46 mmol) and THF (30 mL) was stirred at room temperature under nitrogen for 1 hour as described for 761b, Example 530e, step 1. The crude material was purified by silica gel flash chromatography using a stepwise gradient of EtOAc (50-80%) in hexanes to afford 761c as orange oil (2.0 g, 71%). LRMS (ESI): (calc.) 313.3; (found) 314.0 (MH)⁺, 336.0 (MNa⁺).

### Step 2: 2-(4-fluorobenzyloxy)-N-(3-(4-hydroxyphenyl)propyl)acetamide (765c)

Palladium on charcoal (5% wt, 400 mg) was added to a solution of 761c (2.0 g, 6.4 mmol) in methanol (20 mL) and the reaction was carried out as described for 765a, Example 530e, step 2. The crude material was purified by silica gel flash chromatography using a stepwise gradient of EtOAc (50-80%) in hexanes to obtain 765c as a white solid (1.72 g, 85%). LRMS (ESI): (calc.) 317.4; (found) 318.1 (MH)⁺, 340.0 (MNa⁺).

### Step 3: (R)-tert-butyl 3-(4-(3-(2-(4-fluorobenzyloxy)acetamido)propyl)phenoxy)-pyrrolidine-1-carboxylate (767a)

To a flask containing a suspension of PS—PPh₂ (1.54 g, 3.32 mmol) in THF (20 mL) under nitrogen was added compound 765c (0.700 g, 2.21 mmol). The suspension was stirred for 15 minutes before the addition of DEAD (1.15 g of 40% solution in toluene, 2.65 mmol). After 45 minutes, (S)-(−)-N—BOC-3-pyrrolidinol (0.413 g, 2.21 mmol) was added as a solid. The suspension was stirred for 20 hours and then filtered, and the resin was washed with DCM and the filtrate was concentrated and purified by silica gel chromatography using a stepwise gradient of EtOAc (50-100%) in hexanes to obtain 767a as clear oil (0.302 g, 28%).

(MEOD-d₄) δ (ppm) ¹H: 7.42-7.38 (m, 2H), 7.12-7.09 (m, 2H), 7.07 (d, J=8.8 Hz, 2H), 6.82 (d, J=8.8 Hz, 2H), 4.93 (br s, 1H), 4.56 (s, 2H), 3.91 (s, 2H), 3.54-3.43 (m, 4H), 3.24 (t, J=6.8 Hz, 2H), 2.56 (t, J=7.6 Hz, 2H), 2.15-2.10 (m, 2H), 1.79 (quint. J=7.2 Hz, 2H), 1.46-1.44 (m, 9H). LRMS (ESI): (calc.) 486.3; (found) 487.1 (MH)⁺, 509.1 (MNa⁺).

### EXAMPLE 532a

### R)—N-(3-(4-(1-((1H-indol-3-yl)methyl)pyrrolidin-3-yloxy)phenyl)propyl)-2-(4-fluorobenzyloxy)acetamide (769a)

### Step 1: (R)-2-(4-fluorobenzyloxy)-N-(3-(4-(pyrrolidin-3-yloxy)phenyl)propyl)acetamide (768a)

Compound 767a (0.264 g, 0.543 mmol) was stirred in a solution of trifluoroacetic acid (1 mL) in DCM (1 mL) for 2 hours. The mixture was concentrated and dissolved in EtOAc and washed with saturated NaHCO₃(aq.) (×3), dried (Na₂SO₄) and concentrated to a give amine 768a as brown solid (0.209 g, 99%).

LRMS (ESI): (calc.) 386.5; (found) 387.2 (MH)⁺, 409.1 (MNa⁺).

### Step 2: (R)-N-(3-(4-(1-((1H-indol-3-yl)methyl)pyrrolidin-3-yloxy)phenyl)propyl)-2-(4-fluorobenzyloxy)acetamide (769a)

Amine 768a (84 mg, 0.22 mmol) and indole-3-carboxaldehyde (32 mg, 0.22 mmol) were stirred in THF (1 mL) at room temperature for 1 hour. NaBH(OAc)₃ (46 mg, 0.22 mmol) was added and the reaction was stirred for 20 hours, concentrated, re-dissolved in dichloromethane and washed with water. The organic phase was dried (Na₂SO₄) and concentrated, and the crude material was purified by silica gel flash chromatography using a Biotage 12M column and a slow gradient of MeOH (2-15% methanol) in DCM. It was then further purified by washing the compound in DCM with saturated NaHCO₃ (aq.) (×3), and the organic layer was dried (Na₂SO₄) and concentrated to give pure 769a as a white solid (65 mg, 58%).

(MEOD-d₄) δ (ppm) ¹H, 7.62 (d, J=7.6 Hz, 1H), 7.40-7.37 (m, 2H), 7.34 (d, J=8.0 Hz, 1H), 7.21 (s,1H), 7.11-7.00 (m, 6H), 6.72 (d, J=8.4 Hz, 2H), 4.78 (br s,1H), 4.53 (s, 2H), 3.90 (s, 2H), 3.91 (d, J=13.6 Hz, 1H), 3.86 (d, J=13.2 Hz, 1H), 3.22 (t, J=7.2 Hz, 2H), 2.95 (dd, J=6.4, 10.8 Hz, 1H), 2.88-2.80 (m, 2H), 2.65-2.59 (m, 1H), 2.53 (t, J=8.0 Hz, 2H), 2.29-2.22 (m, 1H), 1.94-1.86 (m, 1H), 1.77 (quint. J=7.2 Hz, 2H).

LRMS (ESI): (calc.) 515.3; (found) 516.2 (MH)⁺, 538.2 (MNa⁺).

### EXAMPLE 533a

### (R)—N-(3-(4-(1-(2-(1H-indol-3-yl)ethyl)pyrrolidin-3-yloxy)phenyl)propyl)-2-(4-fluorobenzyloxy)acetamide (770a)

A suspension of amine 768a (82 mg, 0.21 mmol), 3-(2-bromoethylindole (48 mg, 0.21 mmol) and potassium carbonate (88 mg, 0.639 mmol) was heated to 90° C. in DMF (1 mL) for 3 hours. The suspension was then cooled and filtered and the filtrate taken to dryness. The residue was purified by silica gel flash chromatography using a gradient of MeOH (1-5%) in DCM to afford 770a as yellow oil (39 mg, 35%).

(MEOD-d₄) δ (ppm) ¹H, 7.53 (d, J=7.6 Hz, 1H), 7.41-7.38 (m, 2H), 7.31 (d, J=8.4 Hz, 1H), 7.11-7.05 (m, 6H), 6.99 (t, J=7.6 Hz, 1H), 6.79 (d, J=8.4 Hz, 2H), 4.87 (br s, 1H), 4.55 (s, 2H), 3.91 (s, 2H), 3.24 (t, J=7.2 Hz, 2H), 3.00-2.92 (m, 5H), 2.87-2.80 (m, 2H), 2.68-2.59 (m, 2H), 2.56 (t, J=7.2 Hz, 2H), 2.35-2.28 (m, 1H), 2.00-1.96 (m, 1H), 1.79 (quint. J=7.2 Hz, 2H).

LRMS (ESI): (calc.) 529.3; (found) 530.2 (MH)⁺, 552.2 (MNa⁺).

Example 531b describes the preparation of compound 767b using the same procedures as described for compound 767a, example 531a, step 3, scheme 314, replacing (S)-(−)-N—BOC-3-pyrrolidinol, with (R)-(−)-N—BOC-3-pyrrolidinol. Characterization data are presented in Table 38.

Example 532b describes the preparation of compound 769b using the same procedures as described for compound 769a, example 532a, step 2, scheme 314, replacing 768a, for 768b. Characterization data are presented in Table 38.

Example 533b describes the preparation of compound 770b using the same procedure as described for compound 770a, example 533a, scheme 314, replacing 768a, for 768b. Characterization data are presented in Table 39.

TABLE 39

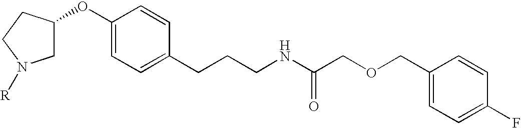

| Ex | No. | R² | Name | Characterization | Scheme |
|---|---|---|---|---|---|
| 531b | 767b | 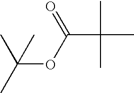 | (S)-tert-butyl 3- (4-(3-(2-(4- fluorobenzyloxy) acetamido)propyl) phenoxy) pyrrolidine-1- carboxylate | (MEOD-d₄) δ (ppm) ¹H: 7.42-7.38 (m, 2H), 7.12-7.08 (m, 2H), 7.07 (d, J=8.8Hz, 2H), 6.82 (d, J=8.8Hz, 2H), 4.92 (br s, 1H), 4.55 (s, 2H), 3.91 (s, 2H), 3.54-3.43 (m, 4H), 3.24 (t, J=6.8Hz, 2H), 2.56 (t, J=7.6Hz, 2H), 2.15-2.10 (m, 2H), 1.79 (quint. | |
| # J=7.2Hz, 2H), 1.46-1.44 (m, 9H). LRMS (ESI): (calc.) 486.3: (found) 487.1 (MH)⁺, 509.1 (MNa⁺). | 314 Ex 531a, step3 | | | | |
| 532b | 769b | 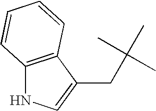 | (S)-N-(3-(4-(1- ((1H-indol-3- yl)methyl) pyrrolidin-3-yloxy) phenyl)propyl)-2-(4- fluorobenzyloxy) acetamide | (MEOD-d₄) δ (ppm) ¹H: 7.62 (d, J=7.6Hz, 1H), 7.41-7.37 (m, 2H), 7.34 (d, J=8.0Hz, 1H), 7.21 (s, 1H), 7.11-7.00 (m, 6H), 6.73 (d, J=8.4Hz, 2H), 4.80 (br s, 1H), 4.54 (s, 2H), 3.90 (s, 2H), 3.91 (d, J=13.6Hz, 1H), 3.86 (d, J=13.2Hz, 1H), 3.22 (t, | |
| # J=7.2Hz, 2H), 2.96 (dd, J=6.4, 10.8Hz, 1H), 2.90-2.81 (m, 2H), 2.65-2.62 (m, 1H), 2.54 (t, J=8.0Hz, 2H), 2.32-2.23 (m, 1H), 1.95-1.87 (m, 1H), 1.78 (quint. J=7.2Hz, 2H). LRMS (ESI): (calc.) 515.26; (found) 516.2 (MH⁺), 538.2 (MNa)⁺ | 314 Ex 532a, step2 | | | | |
| 533b | 770b | 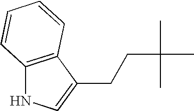 | (S)-N-(3-(4-(1-(2- (1H-indol-3- yl)ethyl)pyrrolidin- 3-yloxy)phenyl) propyl)-2-(4- fluorobenzyloxy) acetamide | (MEOD-d₄) δ (ppm) ¹H: 7.53 (d, J=7.6Hz, 1H), 7.39-7.36 (m, 2H), 7.32 (d, J=8.4Hz, 1H), 7.09-7.05 (m, 6H), 6.99 (t, J=7.6Hz, 1H), 6.78 (d, J=8.4Hz, 2H), 4.85 (br s, 1H), 4.53 (s, 2H), 3.90 (s, 2H), 3.21 (t, J=7.2Hz, 2H), 3.00-2.92 (m, 5H), | |
| # 2.89-2.83 (m, 2H), 2.68-2.62 (m, 2H), 2.55 (t, J=8.0Hz, 2H), 2.33=2.28 (m, 1H), 2.00-1.96 (m, 1H), 1.78 (quint. J=7.6Hz, 2H). LRMS (ESI): (calc.) 529.3; found 530.2 (MH⁺), 552.2 (MNa⁺); | 314 Ex 533a | | | | |

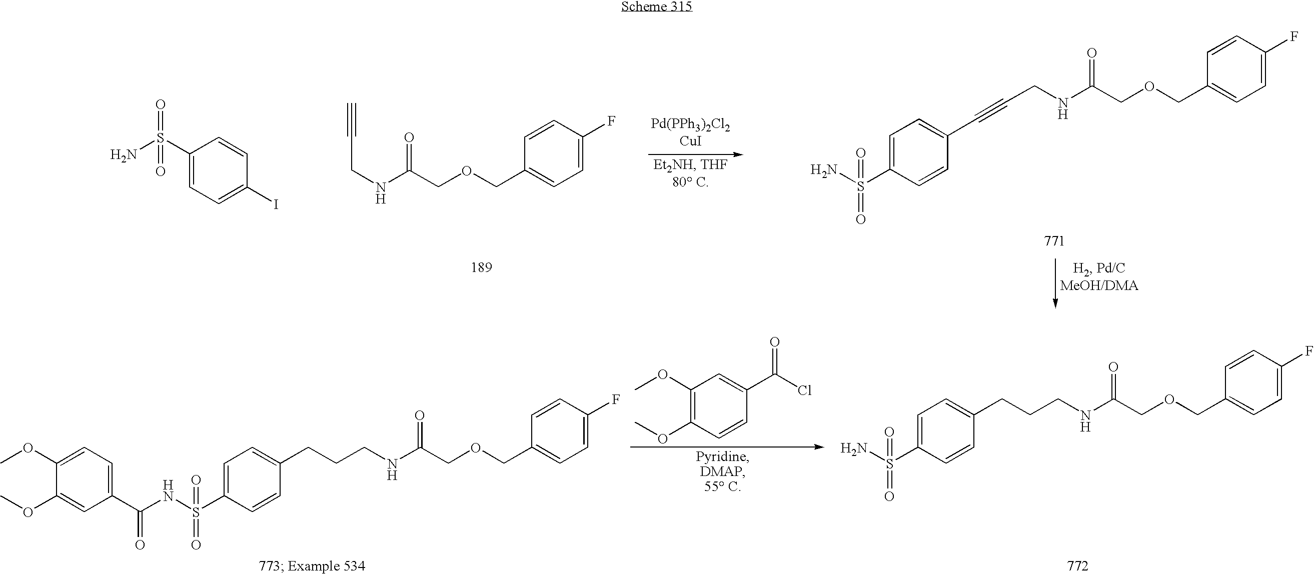

### EXAMPLE 534

### N-(4-(3-(2-(4-fluorobenzyloxy)acetamido)propyl)phenylsulfonyl)-3,4-dimethoxybenzamide (773)

### Step 1: 2-(4-fluorobenzyloxy)-N-(3-(4-sulfamoylphenyl)prop-2-ynyl)acetamide (771)

A solution of alkyne 189 (78 mg, 0.35 mmol), 4-iodobenzenesulfonamide (100 mg, 0.35 mmol), Pd(PPh₃)₂Cl₂ (12 mg, 0.018 mmol), and copper iodide (7 mg, 0.04 mmol) in diethylamine (0.18 mL, 1.8 mmol) and THF (1.5 mL) was stirred under nitrogen for 1 hour at room temperature as described for 761b, Example 530e, step 1, scheme 314. The crude material was purified by column chromatography eluting with a gradient of acetone (25%) in DCM to afford 771 as a white solid (80 mg, 60%).

(DMSO-d₆) δ (ppm) ¹H: 8.41 (t, J=5.6 Hz, 1H), 7.77 (d, J=8.8 Hz, 2H), 7.57 (d, J=8.4 Hz, 2H), 7.45-7.41 (m, 4H), 7.19-7.17 (m, 2H), 4.51 (s, 2H), 4.17 (d, J=6.0 Hz, 2H), 3.94 (s, 2H). LRMS (ESI): (calc.) 376.4; (found) 377.0 (MH)⁺,

### Step 2: 2-(4-fluorobenzyloxy)-N-(3-(4-sulfamoylphenyl)propyl)acetamide (772)

Compound 771 (78 mg, 0.21 mmol) in methanol (1 mL), N,N-dimethylacetamide (1 mL) and palladium on charcoal (5% wt, 30 mg) was hydrogenated as described for 765a, Example 530e, step 2, scheme 314. Filtration of the catalyst and concentration gave 772 as a grey solid (61 mg, 77%).

(DMSO-d₆) δ (ppm) ¹H, 7.88 (t, J=5.6 Hz, 1H), 7.70 (d, J=8.4 Hz, 2H), 7.43-7.39 (m, 2H), 7.37 (d, J=8.4 Hz, 2H), 7.28 (s, 2H), 7.19-7.15 (m, 2H), 4.49 (s, 2H), 3.86 (s, 2H), 3.09 (q, J=6.4 Hz, 2H), 2.61 (t, J=7.6 Hz, 2H), 1.72 (quint., J=7.2 Hz, 2H).

LRMS (ESI): (calc.) 380.4; (found) 381.0 (MH)⁺, 409.1 (MNa⁺),

### Step 3: N-(4-(3-(2-(4-fluorobenzyloxy)acetamido)propyl)phenylsulfonyl)-3,4-dimethoxybenzamide (773)

A solution of Compound 772 (59 mg, 0.16 mmol) and 3,4-dimethoxybenzoylchloride (31 mg, 0.16 mmol) in pyridine (1 mL) was shaken on a mechanical shaker at room temperature for 15 hours. 4-dimethylaminopyridine (6 mg, 0.05 mmol) was then added and the solution was shaken for an additional 12 hours at 55° C. The mixture was concentrated and suspended in DCM and the insoluble material was filtered out. The filtrate was concentrated and purified by preparative HPLC (Aquasil C18 20×250 mm, 50/50 to 95/5 methanol/water (0.05% formic acid) at 10 mL/min in 45 minutes) to afford 773 as an off white semi-crystalline solid (12 mg, 14%).

(MEOD-d₄) □(ppm) ¹H, 7.97 (d, J=8.4 Hz, 2H), 7.51 (dd, J=2.0, 8.4 Hz, 1H), 7.43-7.36 (m, 5H), 7.10-7.04 (m, 2H), 6.97 (d, J=8.4 Hz, 1H), 4.54 (s, 2H), 3.89 (s, 2H), 3.86 (s, 3H), 3.83 (s, 3H), 3.27 (t, J=6.4 Hz, 2H), 2.72 (t, J=7.6 Hz, 2H), 1.88 (quint, J=7.6 Hz, 2H).

LRMS (ESI): (calc.) 544.2; (found) 545.0 (MH)⁺, 567.0 (MNa⁺).

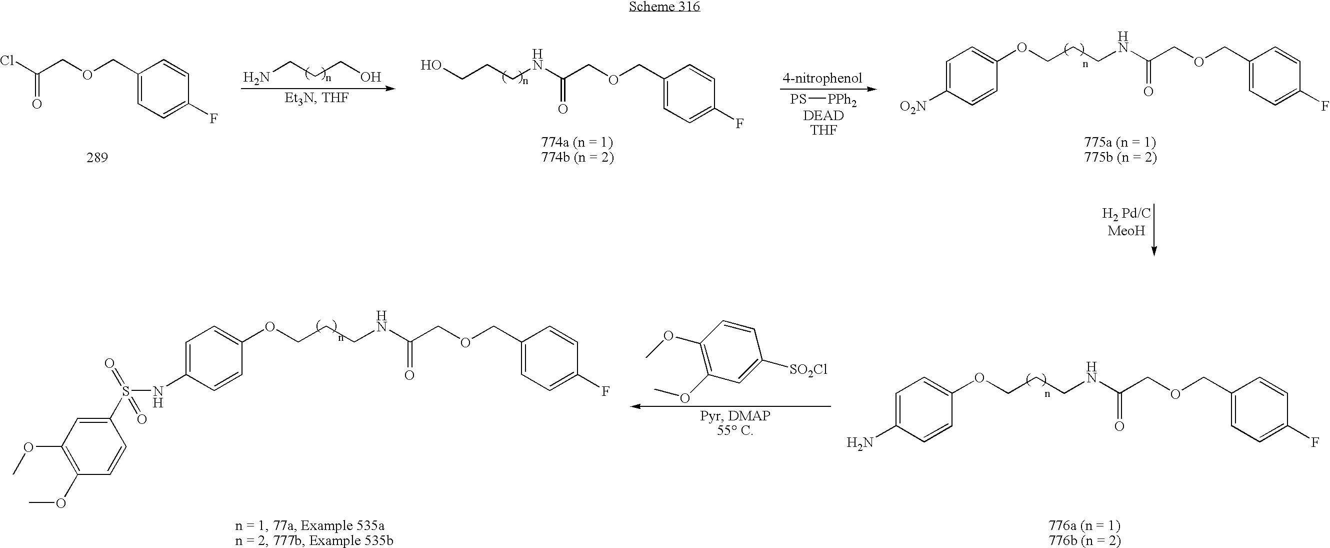

### EXAMPLE 535a

### N-(3-(4-(3,4-dimethoxyphenylsulfonamido)phenoxy)propyl)-2-(4-fluorobenzyloxy)acetamide (777a)

### Step 1: 2-(4-fluorobenzyloxy)-N-(3-hydroxypropyl)acetamide (774a)

A solution of 3-aminopropanol (0.83 mL, 10.8 mmol) and triethylamine (1.5 mL, 10.8 mmol) in THF (5 mL) was added to a solution of acid chloride 289 (10.8 mmol) in THF (20 mL). The mixture was stirred at room temperature for 16 h, then it was diluted with EtOAc and washed with 1 N HCl(aq), dried (Na₂SO₄) and concentrated. The crude material was purified by silica gel flash chromatography using a stepwise gradient of EtOAc (75-100%) in DCM, then a gradient of MeOH (0-15%) in EtOAc to afford 774a as yellow oil (0.920 g, 36%).

(DMSO-d₆) □(ppm) ¹H: 7.81 (t, J=6.0 Hz, 1H), 7.42-7.39 (m, 2H), 7.19-7.14 (m, 2H), 4.49 (s, 2H), 4.48 (t, J=5.2 Hz, 1H), 3.85 (s, 2H), 3.39 (q, J=6.0, 1H), 3.16 (q, J=6.8 Hz, 2H), 1.55 (quint, J=6.8 Hz, 2H).

### Step 2: 2-(4-fluorobenzyloxy)-N-(3-(4-nitrophenoxy)propyl)acetamide (775a)

4-Nitrophenol (0.124 g, 0.892 mmol) was added to a flask containing a suspension of PS—PPh₂ (1.338 g, 1.338 mmol) in THF (7 mL) under nitrogen. The suspension was stirred for 10 minutes before the addition of DEAD (0.466 g of 40% solution in toluene, 1.07 mmol). After 45 minutes a solution of 774a (0.215 g, 0.892 mmol) in THF (1.5 mL) was added. The suspension was stirred 20 h, and the resin was filtered and washed with DCM and MeOH. The filtrate was concentrated and purified by silica gel chromatography using a gradient of EtOAc (50-65%) in DCM to give 775a as yellow oil (171 mg, 53%).

(DMSO-d₆) □(ppm) ¹H: 8.17 (d, J=9.6 Hz, 2H), 7.94 (t, J=6.0 Hz, 1H), 7.42-7.39 (m, 2H), 7.19-7.14 (m, 2H), 7.09 (d, J=9.6 Hz, 2H), 4.49 (s, 2H), 4.10 (t, J=6.0 Hz, 2H), 3.87 (s, 2H), 3.26 (q, J=6.4 Hz, 2H), 1.90 (quint. J=6.4 Hz, 2H).

LRMS (ESI): (calc.) 362.4; (found) 363.0 (MH)⁺, 385.0 (MNa⁺).

### Step 3: N-(3-(4-aminophenoxy)propyl)-2-(4-fluorobenzyloxy)acetamide (776a)

The reaction of 775a (0.150 g, 0.414 mmol) in methanol with palladium on activated charcoal (5% wt, 70 mg) under hydrogen gas atmosphere was carried out as described for 765a, Example 530e, step 2, scheme 314. The crude was purified by silica gel chromatography using a Biotage 12M column eluting with a gradient of EtOAc (50-100%) in DCM to give 776a (114 mg, 83%).

(DMSO-d₆) □(ppm) ¹H, 7.87 (t, J=5.6 Hz, 2H), 7.41-7.38 (m, 2H), 7.18-7.13 (m, 2H), 6.59 (d, J=9.2 Hz, 2H), 6.46 (d, J=9.2 Hz, 2H), 4.58 (s, 2H), 4.49 (s, 2H), 3.86 (s, 2H), 3.80 (t, J=6.4 Hz, 2H), 3.22 (q, J=6.8 Hz, 2H), 1.79 (quint. J=6.4 Hz, 2H).

LRMS (ESI): (calc.) 332.4; (found) 333.0 (MH)⁺, 355.0 (MNa⁺).

### Step 4: N-(3-(4-(3,4-dimethoxyphenylsulfonamido)phenoxy)propyl)-2-(4-fluorobenzyloxy)acetamide. (777a)

Amine 776a (106 mg, 0.319 mmol) was reacted with 3,4-dimethoxybenzenesulfonyl chloride (76 mg, 0.319 mmol), and 4-dimethylaminopyridine (12 mg, 0.096 mmol) in 2.0 mL of pyridine as described for compound 754, Example 523, scheme 312. The crude material was purified by flash chromatography using a gradient of EtOAc (60-90%) in DCM to afford 777a as white solid (154 mg, 90%).

(DMSO-d₆) δ (ppm) ¹H: 9.71 (s, 1H), 7.87 (t, J=6.0 Hz, 1H), 7.40-7.37 (m, 2H), 7.20-7.13 (m, 4H), 7.00 (d, J=8.4 Hz, 1H), 6.93 (d, J=9.2 Hz, 2H), 6.74 (d, J=9.2 Hz, 2H), 4.47 (s, 2H), 3.84 (s, 2H), 3.84 (t, J=6.2 Hz, 2H), 3.75 (s, 3H), 3.70 (s, 3H), 3.20 (q, J=6.4 Hz 2H), 1.80 (quint, J=6.4 Hz, 2H). LRMS (ESI): (calc.) 532.17; (found) 533.0 (MH)⁺, 555.0 (MNa)⁺.

### EXAMPLE 535b

### N-(4-(4-(3,4-dimethoxyphenylsulfonamido)phenoxy)butyl)-2-(4-fluorobenzy-loxy)acetamide. (777b)

Compound 777b was prepared as described for 777a, Example 535a, scheme 316, except using 4-aminobutanol in step 1, in place of 3-aminopropanol. The crude material was purified by silica gel flash chromatography using a Biotage 12S column and a gradient of EtOAc (20-80%) in DCM followed by crystallization from minimum amount of DCM in hexanes to give 777b as white solid (52 mg, 82%).

(DMSO-d₆) □(ppm) ¹H: 9.69 (s, 1H), 7.83 (t, J=6.0 Hz, 1H), 7.41-7.38 (m, 2H), 7.20-7.13 (m, 4H), 7.00 (d, J=8.4 Hz, 1H), 6.93 (d, J=9.2 Hz, 1H), 6.77 (d, J=9.2 Hz, 2H), 4.48 (s, 2H), 3.84 (s, 2H), 3.84 (t, J=6.4 Hz, 2H), 3.77 (s, 3H), 3.70 (s, 3H), 3.12 (q, J=6.0 Hz, 2H), 1.63-1.59 (m, 2H), 1.52-1.48 (m, 2H).

LRMS (ESI): (calc.) 546.18; (found) 547.1 (MH)⁺, 596.0 (MNa)⁺.

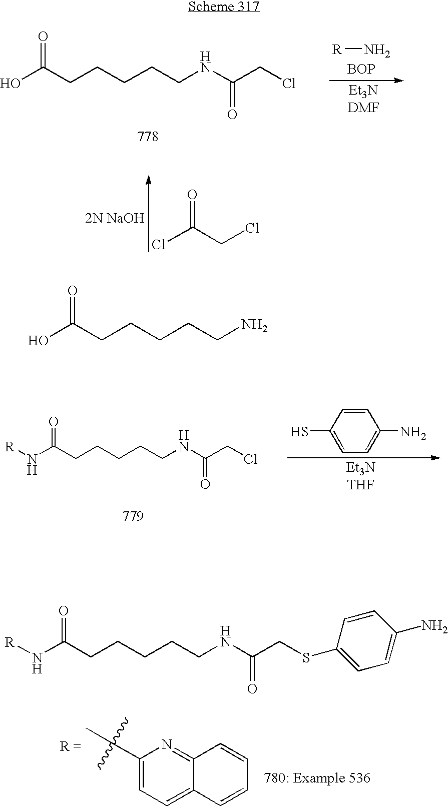

### EXAMPLE 536

### 6-(2-(4-aminophenylthio)acetamido)-N-(quinolin-2-yl)hexanamide (780)

### Step 1: 6-(2-chloroacetamido)hexanoic acid (778)

6-aminocaproic acid (4.00 g, 30.49 mmol) was dissolved in 2N NaOH(aq) (20 mL). The solution was cooled to 0° C. and a third of the chloroacetylchloride (0.75 mL, 9.24 mmol) was added. After most of the precipitate had disappeared, 2N NaOH(aq) (another 5 mL) was added followed by another portion of chloroacetylchloride (0.75 mL, 9.24 mmol). When the reaction mixture had turned clear more 2N NaOH(aq) (5 mL) and the final third of chloroacetylchloride (0.75 mL, 9.24 mmol) were added. The reaction was warmed to room temperature and stirred for an additional hour. It was then acidified to pH˜2 with 6N HCl(aq) and extracted with EtOAc (3×50 mL). The organic extracts were dried (Na₂SO₄) and concentrated to give 778 as a white solid (4.5 g, 78%). LRMS (ESI): (calc.) 207.7; (found) 206.0 (M−H)⁻, 230.0(MNa⁺).

### Step 2: 6-(2-chloroacetamido)-N-(quinolin-2-yl)hexanamide 779

Acid 778 (0.207 g, 0.482 mmol), BOP (0.213 g, 0.483 mmol), 2-aminoquinoline (84 mg, 0.58 mmol), and triethylamine (0.14 mL, 0.966 mmol) in DMF (1 mL) were shaken for 20 h at room temperature. The reaction was diluted with water (45 mL) and extracted with EtOAc (3×20 mL). The organic extracts were washed with saturated NaHCO₃(aq), water, brine, dried (Na₂SO₄), filtered and concentrated. The crude material was purified by flash chromatography using a Biotage 12M column and a gradient of EtOAc (50-100%) in hexanes to afford 779 (58.8 mg, 37%). LRMS (ESI): (calc.) 333.1 (100%), 335.1 (32%); (found) 334.1, 336.1(MH)⁺.

### Step 3: 6-(2-(4-aminophenylthio)acetamido)-N-(quinolin-2-yl)hexanamide (780)

4-aminobenzenethiol (39 mg, 0.31 mmol) in THF (0.22 mL) was added to 779 (51.6 mg, 0.16 mmol) in THF (0.5 mL) followed by triethylamine (430L, 0.31 mmol). The mixture was heated to 75° C. and shaken for 1.5 hours. A small amount of additional 4-aminobenzenethiol (˜10 mg) was added and the mixture was shaken for 72 h at room temperature. The reaction mixture was concentrated and purified first by preparative TLC with ethyl acetate and then by preparative HPLC (Aquasil 20×250 mm C18, 50/50 to 100/0 methanol/water (0.1% formic acid) in 45 min at 10 mL/min) to give 780 as white solid (28 mg, 43%).

(DMSO-d₆) δ (ppm) ¹H, 10.75 (s, 1H), 8.30 (m, 2H), 7.89-7.84 (m, 2H), 7.77 (d, J=8.4 Hz, 1H), 7.68 (dt, J=1.2, 6.8 Hz, 1H), 7.46 (dt, J=1.2, 7.2 Hz, 1H), 7.07 (d, J=8.4 Hz, 2H), 6.47 (d, J=8.8 Hz, 2H), 5.24 (s,2H), 3.30 (s, 2H), 3.01 (q, J=6.0 Hz, 2H), 2.43 (t, J=7.6 Hz, 2H), 1.59 (quint, J=7.2 Hz, 2H), 1.38 (quint, J=8.0 Hz, 2H), 1.26-1.22 (m, 2H).

LRMS (ESI): (calc.) 422.2; (found) 423.1 (MH)⁺.

Examples 536a-e, compounds 780a-e, were prepared as described for compound 780, Example 536, scheme 317, replacing 2-aminoquinoline in step 2, with 4-phenoxyaniline, 4-(4-chlorophenyl)thiazol-2-amine, naphthalen-2-amine, biphenyl-4-ylmethanamine and benzo[d]thiazol-6-amine respectively. Characterization data are presented in Table 40.

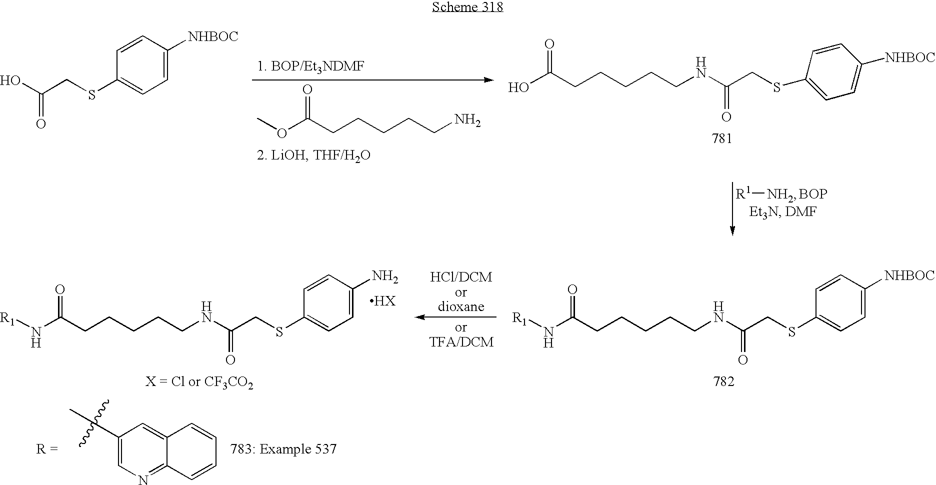

### EXAMPLE 537

### 6-(2-(4-aminophenylthio)acetamido)-N-(quinolin-3-yl)hexanamide dihydrochloride (783)

### Step 1: 6-(2-(4-(tert-butoxycarbonylamino)phenylthio)acetamido)hexanoic acid (781)

Methyl 6-aminohexanoate hydrochloride (3.14 g, 17.1 mmol), BOP (5.46 g, 12.4 mmol), 2-(4-(tert-butoxycarbonylamino)phenylthio)acetic acid (3.50 g, 12.4 mmol) and triethylamine (5.8 mL, 42 mmol) in DMF (30 mL) were stirred at room temperature for 14 hours. The aqueous work-up was divided into two equal batches of the reaction solution. Each batch was diluted with 500 mL of water and then extracted with EtOAc (3×100 mL). The combined organic extracts were washed with 1 N HCl(aq.) (2×100 mL), saturated NaHCO₃(aq.) (100 mL), brine, dried (Na₂SO₄) and concentrated. Purification by silica gel flash chromatography with a gradient of EtOAc (50-70%) in hexanes gave the ester as a white solid (4.53 g, 89%). LRMS (ESI): (calc.) 410.5; (found) 411 (MH⁺), 433(MNa⁺)

The ester from above (3.58 g, 8.73 mmol) in THF (40 mL) H₂O (10 mL) was treated with lithium hydroxide monohydrate (0.733 g, 17.5 mmol) in H₂O (10 mL) and the mixture was stirred for 2 hours at room temperature, then it was acidified to pH 2 with 1 N HCl(aq), diluted with H₂O (200 mL) and extracted with EtOAc (3×50 mL). The organic extracts were washed with brine and concentrated to afford 781 as a white solid (3.33 g, 96%). LRMS (ESI): (calc.) 396.5; (found) 403.2 (MLi⁺), 395.1(M−H)⁻.

### Step 2: tert-butyl 4-(2-oxo-2-(6-oxo-6-(quinolin-3-ylamino)hexylamino)ethylthio)phenyl-carbamate 782

Acid 781 (0.125 g, 0.316 mmol) was reacted with BOP (0.1409, 0.316 mmol), 3-aminoquinoline (55 mg, 0.379 mmol) and triethylamine (0.088 mL, 0.632 mmol) in DMF (1 mL) as described for compound 779, Example 536, step 2, scheme 317. The crude material was purified by silica gel flash chromatography using a Biotage 25M column and a gradient of EtOAc (60-100%) in hexanes to afford 782 (0.83 g, 77%). LRMS (ESI): (calc.) 522.7; (found) 523.3(MH)⁺.

### Step 3: 6-(2-(4-aminophenylthio)acetamido)-N-(quinolin-3-yl)hexanamide 783

Compound 782 (83 mg, 0.159 mmol) was stirred in 3 mL of a freshly prepared solution of HCl in DCM. After 5 hours, the resulting precipitate was filtered and washed with DCM to give 783 as an off-white solid as the HCl salt (55 mg, 70%).

(DMSO-d₆) δ (ppm) ¹H: 11.0 (s, 1H), 9.23 (d, J=2.0 Hz, 1H), 8.99 (d, J=2.4 Hz, 1H), 8.19 (t, J=5.6 Hz, 1H), 8.10 (d, J=4.4 Hz, 1H), 8.11-8.08 (m, 2H), 7.80 (dt, J=1.2, 7.2 Hz, 1H), 7.71 (dt, J=1.2, 8.0 Hz, 1H), 7.40 (d, J=8.4 Hz, 2H), 7.28 (d, J=8.8 Hz, 2H), 3.65 (s, 2H), 3.05 (q, J=6.4 Hz, 2H), 2.43 (t, J=7.2 Hz, 2H), 1.62 (quint, J=7.2 Hz, 2H), 1.42 (quint, J=6.8 Hz, 2H), 1.32-1.26 (m, 2H).

LRMS (ESI): (calc.) 422.5; (found) 423.2 (MH)⁺.

Examples 537a-h, compounds 783a-h, were prepared as described for compound 783, Example 537, scheme 318, replacing 3-aminoquinoline in step 2, with 4-phenylthiazol-2-amine, quinolin-8-amine, (1H-benzo[d]imidazol-2-yl)methanamine, quinolin-6-amine, benzo[d]thiazol-2-amine, 6-methoxybenzo[d]thiazol-2-amine, 4,6-difluorobenzo[d]thiazol-2-amine, and 4-(4-methoxyphenyl)thiazol-2-amine respectively. Characterization data are presented in Table 40.

TABLE 40

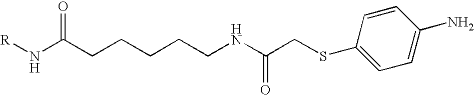

| Ex | No. | R | Name | Characterization | Scheme |
|---|---|---|---|---|---|
| 536a | 780a | 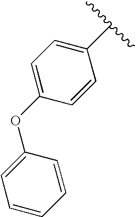 | 6-(2-(4- aminophenyl- thio)acetamido)- N-(4-phenoxy- phenyl)haxanamide | (DMSO-d₆) δ (ppm) ¹H: 9.86 (s, 1H), 7.87 (t, J = 5.6Hz, 1H), 7.58 (d, J =8.8Hz, 2H), 7.36-7.31 (m, 2H), 7.08-7.05 (m, 3H), 6.96-6.91 (m, 4H), 6.47 (d, J = 8.4Hz, 2H), 5.24 (s, 2H), 3.30 (s, 2H), 3.01 (q, J = 6.0Hz, 2H), 2.27 (t, J =7.2Hz, 2H), 1.56 (quint, | |
| # J = 7.6Hz, 2H), 1.38 (quint, J = 7.6Hz, 2H), 1.24 (quint, J = 6.8Hz, 2H). LRMS (ESI): (calc) 463.2; (found) 464.2 (MH)⁺. | 317 Ex 536 | | | | |
| 536b | 780b | 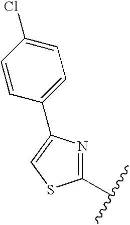 | 6-(2-(4- aminophenyl- thio)acetamido)- N-(4-(4-chloro- phenyl)thiazol-2- yl)hexanamide | (DMSO-d₆) δ (ppm) ¹H: 12.2 (s, 1H), 7.89-7.84 (m, 3H), 7.65 (s, 1H), 7.46 (d, J = 8.8Hz, 2H), 7.07 (d, J = 8.4Hz, 2H), 6.47 (d, J = 8.41Hz, 2H), 5.24 (s, 2H), 3.30 (s, 2H), 3.01 (q, J = 6.0Hz, 2H), 2.43 (t, J = 7.6Hz, 2H), 1.58 (quint, | |
| # J =8.0Hz, 2H), 1.36 (quint, J = 8.0Hz, 2H), 1.22 (m, 2H). LRMS (ESI): (calc) 488.1; (found) 489.1 (MH)⁺. | 317 Ex 536 | | | | |
| 536c | 780c | 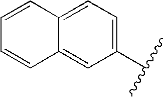 | 6-(2-(4- aminophenyl- thio)acetamido)- N-(naphthalen-2- yl)hexanamide | (DMSO-d₆) δ (ppm) ¹H: 10.04 (s, 1H), 8.28 (m, 1H), 7.87 (t, J = 4.8Hz, 1H), 7.82-7.76 (m, 3H), 7.55 (dd, J = 8.8, 2.0Hz, 1H), 7.43 (dt, J = 1.2, 6.4Hz, 1H), 7.36 (dt, J = 1.6, 6.8Hz, 1H), 7.07 (d, J = 8.8Hz, 2H), 6.47 (d, J = 8.4Hz, 2H), 5.24 (s, 2H), 3.31 (s, 2H), | |
| # 3.01 (q, J = 6.0Hz, 2H), 2.36 (t, J = 7.2Hz, 2H), 1.61 (quint, J = 7.6Hz, 2H), 1.40 (quint, J = 7.6Hz, 2H), 1.31-1.25 (m, 2H). LRMS (ESI): (calc) 421.2; (found) 422.1 (MH)⁺. | 317 Ex 536 | | | | |
| 536d | 780 | 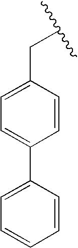 | 6-(2-(4- aminophenyl- thio)acetamido)- N-(biphenyl-4- ylmethyl)hexa- namide | (DMSO-d₆) δ (ppm) ¹H: 8.32 (t, J =5.6Hz, 1H), 7.84 (t, J = 5.6Hz, 1H), 7.60 (d, J = 7.2Hz, 2H), 7.51-7.33 (m, 6H), 7.21 (d, J = 8.0Hz, 1H), 7.07 (d, J =8.8Hz, 2H), 6.47 (J = 8.4Hz, 2H), 5.24 (s, 2H), 4.32 (d, J = 5.6Hz, 2H), 3.30 (s, 2H), 2.98 | |
| # (q, J = Hz, 2H), 2.13 (t, J = 7.6Hz, 2H), 1.51 (quint, J = 7.2Hz, 2H), 1.36 (quint, J = 7.6Hz, 2H), 1.24-1.18 (m, 2H). LRMS (ESI): (calc) 461.2; (found) 462.2 (MH)⁺. | 317 Ex 536 | | | | |
| 536e | 780e | 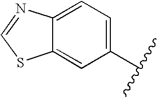 | 6-(2-(4- aminophenyl- thio)acetamido)- N-(benzo[d]thia- zol-6- yl)hexanamide | (DMSO-d₆) δ (ppm) ¹H: 10.12 (s, 1H), 9.22 (s, 1H), 8.53 (d, J = 1.6Hz, 1H), 7.97 (d, J = 8.8Hz, 1H), 7.87 (t, J =5.6Hz, 1H), 7.56 (dd, J = 2.0, 8.8Hz, 1H), 7.07 (d, J = 8.4Hz, 2H), 6.47 (dd; J = 8.4Hz, 2H), 5.24 (s, 2H), 3.31 (s, 2H), 3.01 (q, J = 6.4Hz, | |
| # 2H), 2.33 (t, J =7.6Hz, 2H), 1.59 (quint, J = 7.2Hz, 2H), 1.39 (quint, J = 7.2Hz, 2H), 1.30-1.24 (m, 2H). LRMS (ESI): (calc) 428.1; (found) 429.2 (MH)⁺. | 317 Ex 536 | | | | |
| 537a | 783a | 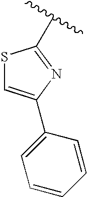 | 6-(2-(4- aminophenyl- thio)acetamido)- N-(4-phenyl- thiazol-2- yl)hexanamide hydrochloride | (DMSO-d₆) δ (ppm) ¹H: 10.12 (s, 1H), 8.12 (t, J = 5.2Hz, 1H), 7.87 (dd, J =0.8, 7.2Hz, 2H), 7.58 (s, 1H), 7.43-7.38 (m, 4H), 7.30 (t, J = 7.2Hz, 1H), 7.19 (d, J = 8.0Hz, 2H), 3.61 (s, 2H), 3.03 (q, J =6.4Hz, 2H), 2.44 (t, J = 7.2Hz, 2H), 1.59 (quint, 7.2Hz, 2H), | |
| # 1.40 (quint, J =7.2Hz, 2H), 1.29-1.23 (m, 2H). LRMS (ESI): (calc) 454.2; (found) 455.2 (MH)⁺. | 318 Ex 537 | | | | |
| 537b | 783b | 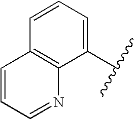 | 6-(2-(4- aminophenyl- thio)acetamido)- N-(quinolin-8- yl)hexanamide dihydrochloride | (DMSO-d₆) δ (ppm) ¹H: 10.0 (s, 1H), 8.91 (dd, J = 1.6, 4.4 Hz, 1H), 8.59 (d, J = 7.6Hz, 1H), 8.41 (dd, J = 1.2, 8.4Hz, 1H), 8.12 (t, J = 5.6Hz, 1H), 7.67-7.62 (m, 2H), 7.56 (t, J = 8.0Hz, 1H), 7.39 (d, J = 8.4Hz, 2H), 7.21 (d, J = 8.4Hz, 2H), 3.61 (s, 2H), 3.05 | |
| # (q, J = 6.0Hz, 2H), 2.56 (t, J = 7.6Hz, 2H), 1.63 (quint, J =7.2Hz, 2H), 1.42 (quint, J = 7.2Hz, 2H), 1.34-1.28 (m, 2H). LRMS (ESI): (calc) 422.2; (found) 423.2 (MH)⁺. | 318 Ex 537 | | | | |
| 537c | 783c | 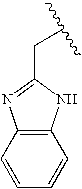 | 6N-((1H- benzo[d]imi- dazol-2- yl)methyl)-6- (2-(4-amino- phenylthio)aceta- mido)hexanamide dihydrochloride | (DMSO-d₆) δ (ppm) ¹H: 8.89 (t, J =5.6Hz, 1H), 8.13 (t, J = 5.2Hz 1H), 7.78-7.75 (m, 2H), 7.52-7.50 (m, 2H), 7.36 (d, J = 8.4Hz, 2H), 7.15 (d, J =8.0Hz, 2H), 4.72 (d, J = 5.2Hz, 2H), 3.60 (s, 2H), 3.02 (q, | |
| # J = 6.4Hz, 2H), 2.23 (t, J = 7.2Hz, 2H), 1.51 (quint, J =7.2Hz, 2H), 1.36 (quint, J = 7.6Hz, 2H), 1.25-1.17 (m, 2H) LRMS (ESI): (calc) 425.2; (found) 460.3 (MCI⁻), | 318 Ex 537 | | | | |
| 537d | 783d | 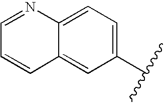 | 6-(2-4- aminophenyl- thio)acetamido)- N-(quinolin-6- yl)hexanamide dihydrochloride | (DMSO-d₆) δ (ppm) ¹H: 10.6 (s, 1H), 9.00 (d, J = 5.2Hz, 1H), 8.83 (d, J =8.4Hz, 1H), 8.66 (d, J = 2.0Hz, 1H), 8.20 (d, J = 8.8Hz, 1H), 8.13 (t, J =5.2Hz, 1H), 8.06 (dd, J = 2.0, 9.2Hz, 2H), 7.83 (dd, J = 5.2, 8.4Hz, 1H), 3.58 (s, 2H), 3.04 (q, J = 6.0Hz, 2H), | |
| # 2.42 (t, J = 7.2Hz, 2H), 1.61 (quint, J = 7.2Hz, 2H), 1.41 (quint, J = 7.2Hz, 2H), 1.28-1.24 (m, 2H) LRMS (ESI): (calc) 422.2; (found) 423.2 (MH)⁺. | 318 Ex 537 | | | | |
| 537e | 783e | 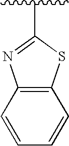 | 6-(2-(4- aminophenyl- thio)acetamido)- N- (benzo[d]thia- zol-2- yl)hexanamide trifluoroacetic acid | (DMSO-d₆) δ (ppm) ¹H: 12.3 (s, 1H), 7.99 (t, J = 5.6Hz, 1H), 7.94 (d, J =8.0Hz, 1H), 7.70 (d, J = 8.0Hz, 1H), 7.41 (dt, J = 1.2, 8.4Hz, 1H), 7.28 (dt, J = 1.2, 8.0Hz, 1H), 7.24 (d, J = 8.4Hz, 2H), 6.85 (d, J = 8.4Hz, 2H), | |
| # 3.47 (s, 2H), 3.02 (q, J = 6..0Hz, 2H), 2.47 (t, J = 7.6Hz, 2H), 1.60 (quint, J = 7.6Hz, 2H), 1.38 (quint, J = 7.6Hz, 2H), 1.29-1.23 (m, 2H) LRMS (ESI): (calc) 428.1; (found) 428.9 (MH⁺), 450.9 (MNa)⁺ | 318 Ex 537 | | | | |
| 537f | 783f | 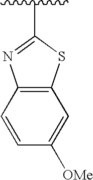 | 6-(2-(4- aminophenyl- thio)acetamido)- N-(6-methoxy- benzo[d]thiazol-2- yl)hexanamide hydrochloride | (DMSO-d₆) δ (ppm) ¹H: 8.15 (t, J =5.2Hz, 1H), 7.59 (d, J = 8.8Hz, 1H), 7.53 (d, J = 2.8Hz, 1H), 7.40 (d, J =8.8Hz, 2H), 7.25 (d, J = 8.4Hz, 2H), 6.99 (dd, J = 2.8, 8.8Hz, 1H), 3.79 (s, 3H), 3.64 (s, 2H), 3.04 (q, | |
| # J = 6.4Hz, 2H), 2.45 (t, J = 7.6Hz, 2H), 1.59 (quint, J = 6.0Hz, 2H), 1.39 (quint, J = 7.2Hz, 2H), 1.29-1.23 (m, 2H) LRMS (ESI): (calc) 458.1; (found) 458.9 (MH³⁰ ), 480.9 (MNa)⁺ | 318 Ex 537 | | | | |
| 537g | 783g | 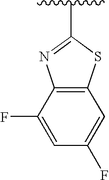 | 6-(2-(4- aminophenyl- thio)acetamido)- N-(4,6- difluoro- benzo[d]thiazol-2- yl(hexanamide trifluoroacetic acid | (DMSO-d₆) δ (ppm) ¹H: 8.15 (t, J =5.2Hz, 1H), 7.59 (d, J = 8.8Hz, 1H), 7.53 (d, J = 2.8Hz, 1H), 7.40 (d, J =8.8Hz, 2H), 7.25 (d, J = 8.4Hz, 2H), 6.99 (dd, J = 2.8, 8.8Hz, 1H), 3.79 (s, 3H), 3.64 (s, 2H), 3.04 (q, J = 6.4Hz, 2H), | |
| # 2.45 (t, J = 7.6Hz, 2H), 1.59 (quint, J = 6.0Hz, 2H), 1.39 (quint, J = 7.2Hz, 2H), 1.29-1.23 (m, 2H) LRMS (ESI): calc 458.1; (found) 458.9 (MH)⁺, 480.9 (MNa)⁺ | 318 Ex 537 | | | | |
| 537h | 784h | 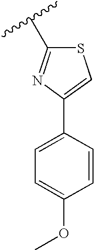 | 6-(2-(4- aminophenyl- thio)acetamido)- N-(4-(4- methoxy- phenyl)thiazol-2- yl)hexanamide hydrochloride | (DMSO-d₆) δ (ppm) ¹H: 12.6 (s, 1H), 8.11 (t, J = 5.6Hz, 1H), 7.89 (d, J =8.8Hz, 2H), 7.41 (s, 1H), 7.38 (d, J =8.8Hz, 2H), 7.18 (d, J = 8.4Hz, 2H), 6.96 (d, J = 8.8Hz, 2H), 3.76 (s, 3H), 3.59 (s, 2H), 3.02 (q, J = 6.0Hz, 2H), 2.40 (t, | |
| # J = 7.6Hz, 2H), 1.56 (quint, J =8.0Hz, 2H), 1.36 (quint, J = 8.0Hz, 2H), 1.24-1.18 (m, 2H) LRMS (ESI): (calc) 484.2; (found) 485.0 (MH)⁺, 507.0 (MNa)⁺. | 318 Ex 537 | | | | |

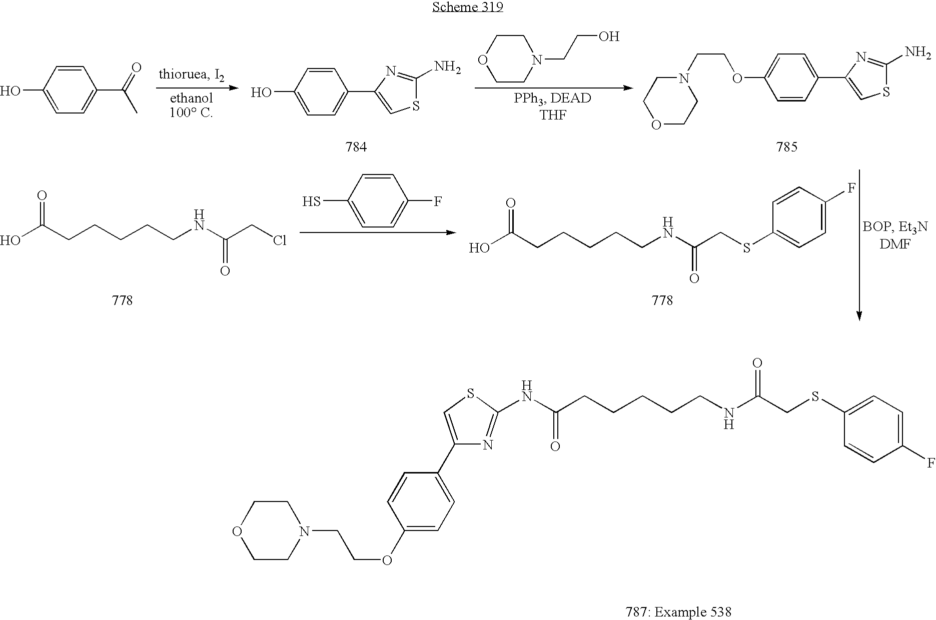

### EXAMPLE 538

### (6-(2-(4-fluorophenylthio)acetamido)-N-(4-(4-(2-morpholinoethoxy)phenyl)thiazol-2-yl)hexanamide) (787)

### Step 1: 4-(2-aminothiazol-4-yl)phenol (784)

Thiourea (2.169, 28.3 mmol), 4-hydroxyacephenone (1.93 g, 14.2 mmol) and iodine (3.60 g, 14.2 mmol) were heated to 100° C. in ethanol (15 mL) inj a pressure tube for 24 hours. The dark colored reaction mixture was cooled and concentrated. The residue was taken up in EtOAc (100 mL) and washed with saturated NaHCO₃(aq) (2×50 mL) and water, dried (Na₂SO₄) and concentrated. The crude material was purified by silica gel flash chromatography using a stepwise gradient of EtOAc (50-70%) in hexanes to afford compound 784 as light yellow oil (1.10 g, 40%). LRMS (ESI): (calc.) 192.2; (found) 193.0 (MH)⁺.

### Step 2: 4-(4-(2-morpholinoethoxy)phenyl)thiazol-2-amine (785)

To a solution of 784 (0.200 g, 1.04 mmol) and 2-hydroxy-ethylmorphorine (0.13 mL, 1.04 mmol) in THF (5 mL) under nitrogen was added triphenylphosphine (0.327 g, 1.25 mmol) followed by DEAD (0.21 mL, 1.35 mmol). The reaction was stirred at room temperature for 20 hours and was concentrated. The residue was diluted with EtOAc and washed with water and then 1 N HCl(aq). The acidic wash was basified to pH 8 with by the addition of solid NaHCO₃ to give a cloudy suspension which was extracted into EtOAc and the extracts were dried (Na₂SO₄), concentrated and purified by silica gel flash chromatography using a Biotage 12M column and a gradient of MeOH (5-10%) in DCM to give compound 785 as white solid (0.197 g, 62%). LRMS (ESI): (calc.) 305.4; (found) 306.1 (MH)⁺.

### Step 3: 6-(2-(4-fluorophenylthio)acetamido)hexanoic acid (786)

To a solution of compound 778, Example 536, step 1, scheme 317 (0.377 g, 1.82 mmol) and triethylamine (0.63 mL, 4.55 mmol) in THF (4 mL) was added 4-fluorobenzenethiol (0.29 mL, 2.73 mmol). The white precipitate that formed was filtered off and the filtrate was diluted with EtOAc, and the organic phase was washed with 1 N HCl(aq) and extracted with saturated NaHCO₃(aq). The NaHCO₃ extracts were acidified to pH 2 with concentrated HCl (aq) and the aqueous layer was extracted with EtOAc, and the organic extracts were dried (Na₂SO₄), filtered and concentrated to give acid 786 as semi-crystalline white solid (0.485 g, 89%). LRMS (ESI): (calc.) 299.4; (found) 298.0 (M−H)⁻.

### Step 4: 6-(2-(4-fluorophenylthio)acetamido)-N-(4-(4-(2-morpholinoethoxy)phenyl)thiazol-2-yl)hexanamide (787)

A solution of compound 786 (0.101 g, 0.338 mmol), 785 (0.103 g, 0.338 mmol), BOP (0.149 g, 0.338 mmol), and triethylamine (0.094 mL, 0.676 mmol) was stirred in DMF (1.0 mL) at room temperature for 17 h as described for compound 781, Example 537, scheme 318. The crude material was purified by silica gel flash chromatography using a Biotage 12M column and a gradient of MeOH (1-10%) in DCM containing 1% acetic acid. The material was further purified by preparative HPLC (Aquasil 20×250 mm C18, 40/60 to 90/10 methanol/water (0.05% formic acid) in 45 min at 10 mL/min) to give pure 787 as a white solid (51 mg, 26%). ¹H NMR: (DMSO-d₆) □(ppm): 12.3 (s,1H), 8.15 (t, J=5.6 Hz, 1H), 7.88 (d, J=8.8 Hz, 2H), 7.50-7.46 (m, 3H), 7.27-7.22 (m, 2H), 7.06 (d, J=8.8 Hz, 2H), 4.19 (t, J=6.0 Hz, 2H), 3.67-3.64 (m, 6H), 3.11 (q, J=6.0 Hz, 2H), 2.77 (t, J=5.6 Hz, 2H), 2.56-2.54 (m, 4H), 2.49 (t, J=7.2 Hz, 2H), 1.64 (quint, J=7.6 Hz, 2H), 1.44 (quint, 7.6 Hz, 2H), 1.32-1.27 (m, 2H).

LRMS (ESI): (calc.) 586.2; (found) 587.2 (MH)⁺, 609.0 (MNa)⁺.

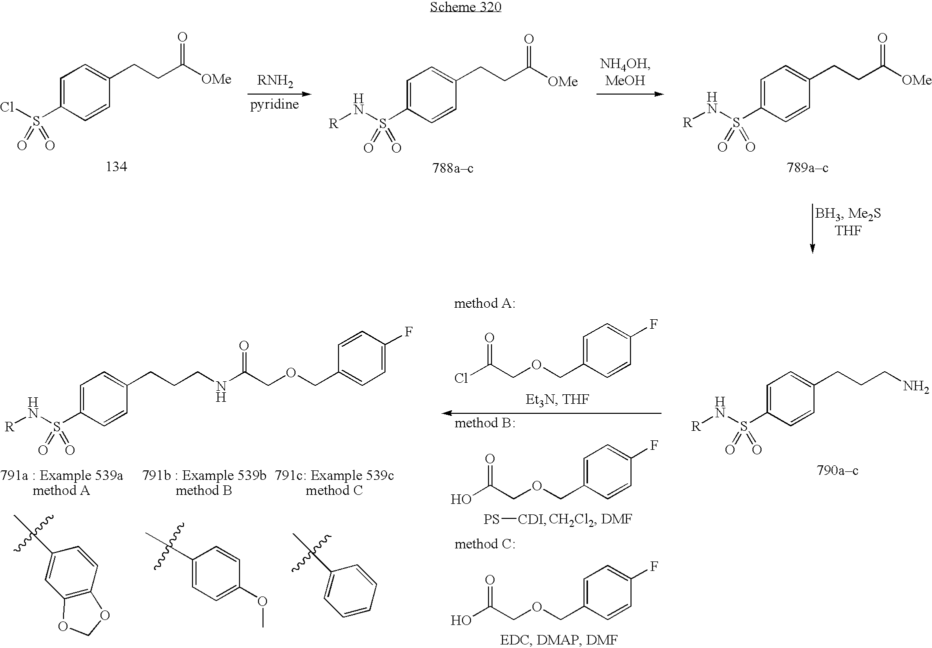

### EXAMPLE 539a

### N-(3-(4-(N-benzo[d][1,3]dioxol-5-ylsulfamoyl)phenyl)propyl)-2-(4-fluorobenzyloxy)acetamide (791a)

### Step 1: methyl 3-(4-(N-benzo[d][1,3]dioxol-5-ylsulfamoyl)phenyl)propanoate 788a

Methyl 3(4-chlorosulphonyl)phenylpropionate 134 (1.00 g, 3.81 mmol) was added drop-wise to a stirred solution of 3,4-(methylenedioxy)aniline (522 mg, 3.81 mmol) in pyridine (4 mL) at 0° C., and was stirred at room temperature for 16 h. The reaction mixture was quenched with a saturated solution of ammonium chloride in water, and pyridine was evaporated, and the aqueous mixture was extracted with EtOAc. The organic extract was dried (MgSO₄), filtered, and evaporated. Crude 788a was used without further purification. (CDCl₃) δ (ppm) ¹H, 7.64 (d, J=8.4 Hz, 2H), 7.27-7.24 (m, 2H), 6.66 (d, J=2.2 Hz, 1H), 6.63 (d, J=8.2 Hz, 1H), 6.52 (bs, 1H), 6.40 (dd, J=8.2 and 2.2 Hz, 1H), 5.94 (s, 2H), 3.66 (s, 3H), 2.99 (t, J=7.6 Hz, 2H), 2.63 (t, J=7.8 Hz, 2H). LRMS (ESI): (calc) 363.1; (found) 362.0 (M−H)⁻.

### Step 2: 3-(4-(N-benzo[d][1,3]dioxol-5-ylsulfamoyl)phenyl)propanamide 789a

To a stirred solution of 788a (0.30 mmol) in MeOH (2 mL) was added ammonium hydroxide (2 mL). The reaction was stirred at room temperature for 16 h, then it was taken to dryness and triturated with ethyl ether to give 789a (67 mg, 64%).

(DMSO-d₆) δ (ppm) ¹H: 7.58 (d, J=8.4 Hz, 2H), 7.35 (d, J=8.6 Hz, 2H), 7.26 (bs, 1H), 6.76 (bs, 1H), 6.72 (d, J=8.4 Hz, 1H), 6.63 (d, J=2.2 Hz, 1H), 6.44 (dd, J=8.4 and 2.2 Hz, 1H), 5.93 (s, 2H), 2.82 (t, J=7.8 Hz, 2H), 2.33 (t, J=8.0 Hz, 2H). LRMS (ESI): (calc) 348.0; (found) 346.9 (M−H)⁻.

### Step 3: 4-(3-aminopropyl)-N-(benzo[d][1,3]dioxol-5-yl)benzenesulfonamide (790a)

To a stirred solution of 789a (127 mg, 0.36 mmol) in THF (1.8 mL) was added borane-methyl sulfide (1.5 mL of 2.0M in THF) and the reaction was stirred at 65° C. for 16 h as described for compound 736, example 514, scheme 308, step 2. Crude 790a was used without further purification. LRMS (ESI): (calc) 334.1; (found).335.1 (MH)⁺

### Step 4: N-(3-(4-(N-benzo[d][1,3]dioxol-5-ylsulfamoyl)phenyl)propyl)-2-(4-fluorobenzyloxy)acetamide (791a)

Method A:

To a stirred solution of 790a (0.27 mmol) in THF (0.5 mL) was added triethylamine (0.11 mL, 0.81 mmol). The solution was cooled to 0° C., and then a solution of acid chloride 289 (0.32 mmol) in THF (0.5 mL) was added as described for compound 723, example 507, scheme 302, step 4. The crude material was purified by silica gel column chromatography with gradient of EtOAc (60-100%) in hexane. The isolated product was purified again by prep-hplc with gradient of methanol (50-100%) in water to give 791a (6 mg, 5%).

(CD₃CN) δ (ppm) ¹H: 7.63 (d, J=8.4 Hz, 2H), 7.42 (dd, J=8.8 and 5.3 Hz, 2H), 7.35 (d, J=8.4 Hz, 2H), 7.13 (tt, J=9.0 and 2.9 Hz, 2H), 6.91 (bs, 1H), 6.69-6.67 (m, 2H), 6.47 (dd, J=6.1 and 2.2 Hz, 2H), 5.93 (s, 2H), 4.54 (s, 2H), 3.90 (s, 2H), 3.21 (q, J=7.4 Hz, 2H), 2.68 (t, J=7.4 Hz, 2H), 1.79 (qi, J=7.4 Hz, 2H). LRMS (ESI): (calc) 500.1; (found).499.0 (M−H)⁻.

### EXAMPLE 539b

### 2-(4-fluorobenzyloxy)-N-(3-(4-(N-(4-methoxyphenyl)sulfamoyl)phenyl)propyl)acetamide (791b)

Steps 1 to 3 to prepare 4-(3-aminopropyl)-N-(4-methoxyphenyl)benzenesulfonamide 790b, were carried-out following the same procedures as desribed for 790a, in example 539a, scheme 320, except in step 1 p-anisidine was used in place of 3,4-(methylenedioxy)aniline.

### Step 4: 2-(4-fluorobenzyloxy)-N-(3-(4-(N-(4-methoxyphenyl)sulfamoyl)phenyl)propyl)-acetamide 791b

Method B:

To a stirred solution PS-CDI (428 mg, 0.60 mmol) in DCM (3 mL) was added acid 140 (83 mg, 0.45 mmol). The mixture was stirred for 10 minutes at room temperature. A solution of amine 790b (97 mg, 0.30 mol) in DMF (1 mL) was added. The reaction was stirred at room temperature for 16 h. The resin was filtered and rinsed with DCM. The solvent was evaporated and the residue was purified by prep-hplc with gradient of methanol (40-100% in water to give 791b (10 mg, 7%). (CD₃CN) □ (ppm) ¹H: 7.60 (d, J=8.6, 2H), 7.42 (dd, J=8.8 and 5.5 Hz, 2H), 7.33 (d, J=8.6 Hz, 2H), 7.13 (t, J=9.0 Hz, 2H), 6.99 (d, J=9.2 Hz, 2H), 6.91 (bs, 1H), 6.80 (d, J=9.0 Hz, 2H), 4.54 (s, 2H), 3.90 (s, 2H), 3.72 (s, 3H), 3.21 (q, J=6.5 Hz, 2H), 2.67 (t, J=7.6 Hz, 2H), 1.78 (qi, J=7.2 Hz, 2H). LRMS (ESI): (calc) 486.1; (found) 485.0 (M−H)⁻.

### EXAMPLE 539c

### 2-(4-fluorobenzyloxy)-N-(3-(4-(N-phenylsulfamoyl)phenyl)propyl)acetamide (791c)

Steps 1 to 3 to prepare 4-(3-aminopropyl)-N-phenylbenzenesulfonamide 790c, were carried out following the same procedures as desribed for 790a, in example 539a, scheme 320, except in step 1 aniline was used in place of 3,4-(methylenedioxy)aniline.

### Step 4: 2-(4-fluorobenzyloxy)-N-(3-(4-(N-phenylsulfamoyl)phenyl)propyl)acetamide (791c)

Method C:

To a stirred solution of acid 140 (47 mg, 0.26 mmol) in dimethylformamide (1 mL) was added N-(3-Dimethylaminopropyl)-N′-ethylcarbodiimide hydrochloride (81 mg, 0.42 mmol). The mixture was stirred for 5 minutes at room temperature. 4-Dimethylaminopyridine (34 mg, 0.26 mmol) was added. A solution of amine 790c (82 mg, 0.26 mol) in dimethylformamide (0.7 mL) was added. The reaction was stirred at room temperature for 16 h. The reaction mixture was quenched with a saturated solution of sodium carbonate in water. The aqueous mixture was extracted with EtOAc, dried (MgSO₄), filtered, and concentrated. The residue was purified by prep-hplc with gradient of methanol (40-100% in water to give 791c (5 mg, 4%). (CD₃CN) δ (ppm) ¹H: 7.69 (d, J=8.2 Hz, 2H), 7.12 (dd, J=8.6 and 5.7 Hz, 2H), 7.34 (d, J=8.2 Hz, 2H), 7.26 (dd, J=8.6 and 7.0 Hz, 2H), 7.16-7.08 (m, 5H), 6.90 (bs, 1H), 4.54 (s, 2H), 3.89 (s, 2H), 3.20 (q, J=7.4 Hz, 2H), 2.66 (t, J=7.6 Hz, 2H), 1.77 (qi, J=7.4 Hz, 2H). LRMS (ESI): (calc) 456.2; (found) 455.0 (M−H)⁻.

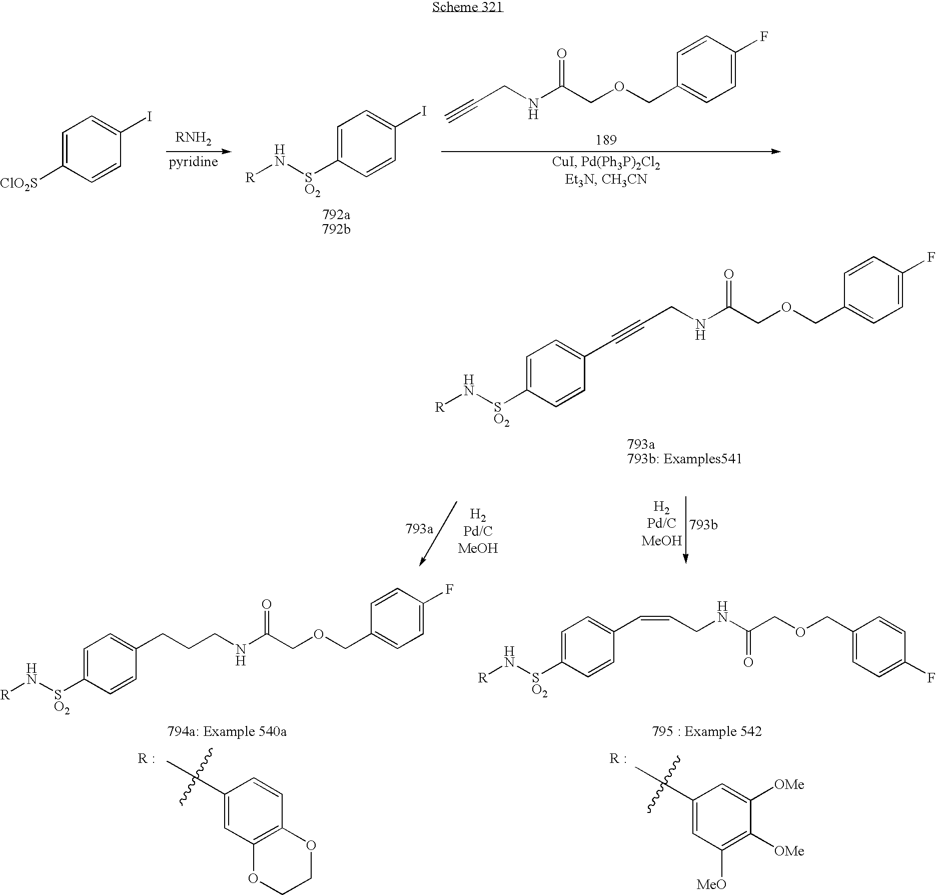

### EXAMPLE 540a

### N-(3-(4-(N-(2,3-dihydrobenzo[b][1,4]dioxin-6-yl)sulfamoyl)phenyl)propyl)-2-(4-fluorobenzyloxy)acetamide (794a)

### Step 1: N-(2,3-dihydrobenzo[b][1,4]dioxin-6-yl)-4-iodobenzenesulfonamide (792a)

To a stirred solution of 1,4-benzodioxan-6-amine (0.34 mL, 2.74 mmol) in pyridine (4 mL) was added 4-iodobenzenesulfonyl chloride (830 mg, 2.74 mmol) as described for compound 788a, example 539a, step 1, scheme 320. The crude was purified by silica gel column chromatography with gradient of EtOAc (20-60%) in hexane to give 792a (937 mg, 82%).

(DMSO-d₆) δ (ppm) ¹H, 10.03 (s,1H), 7.92 (d, J=8.6 Hz, 2H), 7.43 (d, J=8.6 Hz, 2H), 6.70 (d, J=8.6 Hz, 1H), 6.54 (d, J=2.5 Hz, 1H), 6.49 (dd, J=8.8 and 2.5 Hz, 1H), 4.15-4.12 (m, 4H). LRMS (ESI): (calc0 417.0; (found) 415.8 (M−H)⁻.

### Step 2: N-(3-(4-(N-(2,3-dihydrobenzo[b][1,4]dioxin-6-yl)sulfamoyl)phenyl)prop-2-ynyl)-2-(4-fluorobenzyloxy)acetamide (793a)

To a stirred solution of 792a (209 mg, 0.50 mmol) in acetonitrile (1.5 mL) was added alkyne 189 (133 mg, 0.60 mmol), triethylamine (0.10 mL, 0.75 mmol), dichlorobis(triphenylphosphine)palladium(II) (18 mg, 0.03 mmol), and copper(I) iodide (10 mg, 0.05 mmol). The mixture was stirred for 3 h at room temperature. The mixture was concentrated and the residue was purified by silica gel column chromatography with gradient of ethyl acetate (40-80%) in hexane to give 793a (202 mg, 79%).

(DMSO-d₆) δ (ppm) ¹H: 10.0 (s, 1H), 8.39 (t, J=6.1 Hz, 1H), 7.64 (d, J=8.6 Hz, 2H), 7.54 (d, J=8.6 Hz, 2H), 7.42 (dd, J=8.8 and 5.7 Hz, 2H), 7.17 (t, J=9.0 Hz, 2H), 6.68 (d, J=8.6 Hz, 1H), 6.52 (d, J=2.5 Hz, 1H), 6.47 (dd, J=8.6 and 2.5 Hz, 1H), 4.51 (s, 2H), 4.16-4.12 (m, 6H), 3.93 (s, 2H). LRMS (ESI): (calc) 510.1; (found) 509.0 (M−H)⁻

### Step 3: N-(3-(4-(N-(2,3-dihydrobenzo[b][1,4]dioxin-6-yl)sulfamoyl)phenyl)propyl)-2-(4-fluorobenzyloxy)acetamide (794a)

Compound 793a (159 mg, 0.31 mmol) in methanol (1.6 mL) and 10% palladium on charcoal (16 mg) was hydrogenated under 1 atm of H₂ gas. The residue was purified by silica gel column chromatography with gradient of ethyl acetate (40-80%) in hexane to give 794a (97 mg, 61%).

(DMSO-d₆) δ (ppm) ¹H, 9.91 (s, 1H), 7.86 (t, J=6.1 Hz, 1H), 7.59 (d, J=8.2 Hz, 2H), 7.40 (dd, J=8.4 and 2.0 Hz, 2H), 7.35 (d, J=8.4 Hz, 2H), 7.16 (t, J=9.0 Hz, 2H), 6.67 (d, J=8.6 Hz, 1H), 6.55 (d, J=2.5 Hz, 1H), 6.49 (dd, J=8.6 and 2.5 Hz, 1H), 4.48 (s, 2H), 4.14-4.11 (m, 4H), 3.84 (s, 2H), 3.08 (q, J=6.5 Hz, 2H), 2.58 (t, J=7.2 Hz, 2H), 1.69 (qi, J=7.6 Hz, 2H). LRMS (ESI): (calc) 514.2; (found) 513.0 (M−H)⁻.

Examples 539d-e, compounds 791d-e were prepared as described for compound 791b Example 539b, scheme 320, except in step 1 replacing p-anisidine with 3-methoxyaniline and 4-(4-methylpiperazin-1-yl)aniline respectively. Characterization data are presented in Table 41

Examples 540b-j, compounds 794b-j were prepared as described for compound 794a Example 540a, scheme 321. Characterization data are presented in Table 41.

TABLE 41

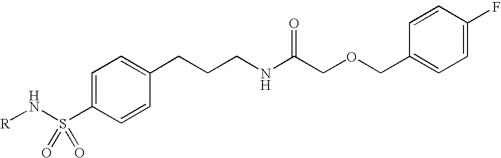

| Ex | Cpd | R | Name | Characterization | Scheme |
|---|---|---|---|---|---|
| 539d | 791d | 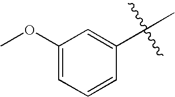 | 2-(4- fluorobenzyloxy)-N- (3-(4-(N-(3- methoxyphenyl)sulfa- moyl)phenyl)pro- pyl)acetamide | (CD₃CN) δ (ppm) ¹H: 7.71 (d, J=8.4Hz, 2H), 7.41 (dd, J=8.8 and 5.5Hz, 2H), 7.34 (d, J=8.4Hz, 2H), 7.17-7.07 (m, 3H), 6.92 (bs, 1H), 6.70-6.63 (m, 3H), 4.54 (s, 2H), 3.90 (s, 2H), 3.71 (s, 3H), 3.21 (q, J=6.5Hz, 2H), 2.66 (t, J=8.8Hz, 2H), 1.77 | |
| # (qi, J=7.4Hz, 2H). LRMS (ESI): 486.1 (calc) 485.1 (M−) (found). | 320 Ex 539b Step 1-4 | | | | |
| 539e | 791e | 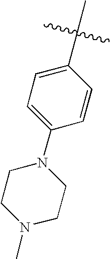 | 2-(4- fluorobenzyloxy)-N- (3-(4-(N-(4-(4- methylpiperazin-1- yl)phenyl)sulfa- moyl)phenylA)pro- pyl)acetamide | (CD₃CN) δ (ppm) ¹H: 8.25 (s, 1H), 7.61 (d, J=8.4Hz, 2H), 7.43-7.37 (m, 2H), 7.32 (d, J=8.6Hz, 2H), 7.15-7.08 (m, 2H), 6.94 (d, J=9.0Hz, 2H), 6.79 (d, J=9.2Hz, 2H), 4.54 (s, 2H), 3.99 (s, 1H), 3.90 (s, 2H), 3.20 (q, J=6.5Hz, 2H), | |
| # 3.17-3.14 (m, 4H), 2.71-2.64 (m, 6H), 2.40 (s, 3H), 1.78 (qi, J=4.2Hz, 2H). LRMS (ESI): 554.2 (calc) 555.2 (MH)+(found). | 320 Ex 539b Step 1-4 | | | | |
| 540b | 794b | 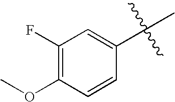 | N-(3-(4-(N-(3-fluoro- 4-methoxy- phenyl)sulfa- moyl)phenyl)propyl)- 2-(4-fluorobenzyl- oxy)acetamide | (DMSO-d₆) δ (ppm) ¹H: 10.09 (s, 1H), 7.86 (t, J=5.9Hz, 1H), 7.59 (d, J=8.7Hz, 2H), 7.40 (dd, J=8.8 and 5.7Hz, 2H), 7.35 (d, J=8.4Hz, 2H), 7.16 (t, J=9.0Hz, 2H), 7.00 (t, J=9.2Hz, 1H), 6.88 (dd, J=12.7 and 2.5Hz, 1H), | |
| # 6.79-6.76 (m, 1H), 4.48 (s, 2H), 3.84 (s, 2H), 3.71 (s, 3H), 3.07 (q, J=6.5Hz, 2H), 2.58 (t, J=7.6Hz, 2H), 1.69 (qi, J=7.4Hz, 2H). LRMS (ESI): 504.1 (calc) 503.0 (M−) (found) | 321 Ex 540a | | | | |
| 540c | 794c | 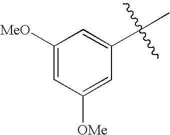 | N-(3-(4-(N-(3,5- dimethoxyphenyl)sulfa- moyl)phenyl)propyl)- 2-(4-fluoro- benzyloxy)acetamide | (DMSO-d₆) δ (ppm) ¹H: 10.24 (s, 1H), 7.84-7.87 (m, 1H), 7.66-7.68 (m, 2H), 6.24 (d, 2H, J=2Hz), 6.12 (t, 1H, J=2Hz), 4.47 (s, 2H), 3.83 (s, 3H), 3.60 (s, 6H), 3.05-3.10 (m, 2H), 2.56-2.60 (m, 2H), 1.65-1.72 (M, 2H). LRMS (ESI): 516.17 (calc) 515.0 (M−) (found) | 321 Ex 540a |
| 540d | 794d | 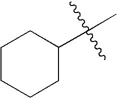 | N-(3-(4-(N- cyclohexylsulfa- moyl)phenylpropyl-2- (4-fluoro- benzyloxy)acetamide | (CDCl₃) δ (ppm) ¹H: 7.76 (d, 1H, J=8.4Hz), 7.28-7.31 (m, 4H), 7.03-7.07 (m, 2H), 6.60 (bs, 1H), 4.51 (s, 2H), 3.95 (s, 2H), 3.30-3.35 (m, 2H), 3.08-3.16 (m, 1H), 2.68-2.72 (m, 2H), 1.82-1.90 (m, 2H), 1.60-1.67 (m, 2H), 1.48-1.58 (m, 1H), 1.07-1.17 (m, 5H). | |
| # LRMS (ESI): 462.20 (calc) 461.0 (M−) (found). | 321 Ex 540a | | | | |
| 540e | 794e | 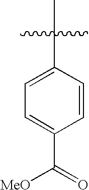 | methyl 4-(4-(3-(2-(4- fluorobenzyl- oxy)acetamido) propyl)phenyl- sulfonamido) benzoate | (CDCl₃) δ (ppm) ¹H: 7.89 (d, 2H, J=8.8Hz), 7.77-7.71 (m, 3H), 7.27-7.30 (m, 2H), 7.22 (d, 2H, J=8.2Hz), 7.15 (d, 2H, J=8.8Hz), 7.04 (t, 2H, J=8.6Hz), 6.60-6.63 (m, 1H), 4.50 (s, 2H), 3.96 (s, 2H), 3.86 (s, 3H), 3.30 (q, 2H, J=6.8Hz), 2.64 (t, 2H, J=7.47Hz), | |
| # 7.82 (qi, 2H, J=7.4Hz). LRMS (ESI): 514.16 (calc) 513.0 (M−) (found) | 321 Ex 540a | | | | |
| 540f | 794f | 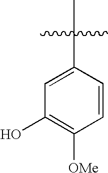 | 2-(4- fluorobenzyloxy)-N- (3-(4-(N-(3-hydroxy- 4- methoxyphenyl)sulfa- moyl)phenyl)pro- lyl)acetamide | (DMSO-d₆) δ (ppm) ¹H: 9.75 (bs, 1H), 9.06 (s, 1H), 7.85 (t, 1H, J=6.0Hz), 7.57 (d, 2H, J=8.4Hz), 7.38-7.42 (m, 2H), 7.33 (d, 2H, J=8.4Hz), 7.16 (t, 2H, J=8.9Hz) 6.70 (d, 1H, J=8.6Hz), 6.55 (d, 1H, J=2.7Hz), 6.39-6.42 (m, 1H), 4.47 (s, 2H), 3.84 | |
| # (s, 2H), 3.62 (s, 3H), 3.05-3.09 (m3H), 2.57 (t, 2H, J=7.4Hz), 1.67-1.72 (m, 2H). LRMS (ESI): 502.16 (calc) 501.0 (M−) (found) | 321 Ex 540a | | | | |
| 540g | 794g | 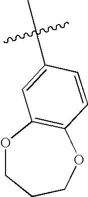 | N-(3-(4-(N-(3,4- dihydro-2H- benzo[b][1, 4]dioxepin-7- yl)sulfamoyl)phenyl propyl)-2-(4- fluorobenzyl- oxy)acetamide | (DMSO-d₆) δ (ppm) ¹H: 10.05 (bs, 1H), 7.85 (t, 2H, J=6.9Hz), 7.60 (d, 2H, J=8.2Hz), 7.33-7.41 (m, 4H), 7.15 (t, 2H, J=8.9Hz), 6.78 (d, 1H, J=8.4Hz), 6.58-6.63 (m, 2H), 4.47 (s, 2H), 3.95-4.01 (m, 4H), 3.83 (s, 2H), 3.06 (q, 2H, J=6.4Hz), | |
| # 2.57 (t, 2H, J=7.8Hz), 1.96-2.00 (m, 2H), 1.68 (qi, 2H, J=7.4Hz). LRMS (ESI): 528.17 (calc) 527.0 (M−) (found) | 321 Ex 540a | | | | |
| 540h | 794h | 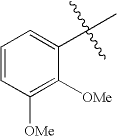 | N-(3-(4-(N-(2,3- dimethoxyphenyl)sul- famoyl)phenyl)propyl)- 2-(4- fluorobenzyl- oxy)acetamide | (DMSO-d₆) δ (ppm) ¹H: 9.57 (bs), 7.88 (t, 1H, J=5.8Hz), 7.66 (d, 2H, J=8.4Hz), 7.38-7.44 (m, 2H), 7.35 (d, 2H, J=8.4Hz), 7.17 (d, 2H, J=8.8Hz), 6.89-6.96 (m, 2H), 6.76 (dd, 1H, J=2.7 and 7.0Hz), 4.49 (s, 2H), 3.85 (s, 2H), 3.71 (s, 3H), 3.35 (s, 3H), 3.07 (q, 2H, J=6.6Hz), | |
| # 2.58 (t, 2H, J=7.8Hz), 1.68 (qi, 2H, J=7.2Hz). LRMS (ESI): 516.17 (calc) 515.0 (M−) (found) | 321 Ex 540a | | | | |
| 540i | 794i | 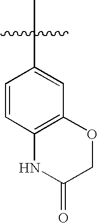 | 2-(4- fluorobenzyloxy)-N- (3-(4-(N-(3-oxo-3,4- dihydro-2H- benzo[b][1,4]oxazin- 7- yl)sulfamoyl)phenyl) propyl)acetamide | (DMSO-d₆) δ (ppm) ¹H: 10.87 (s, 1H), 10.57 (s, 1H), 7.85 (s, 1H), 7.60 (d, 2H, J=8.2Hz), 7.33-7.41 (m, 4H), 7.15 (t, 2H, J=8.8Hz), 6.65 (m, 3H), 4.46 (d, 4H, J=5.0Hz), 3.84 (s, 2H), 3.07 (q, 2H, J=J=6.4Hz), 2.57 | |
| # (t, 2H, J=7.6Hz), 1.68 (qi, 2H, J=7.2Hz). LRMS (ESI): 527.15 (calc) 526.0 (M−) (found) | 321 Ex 540a | | | | |
| 540j | 794j | 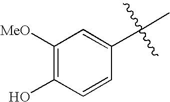 | 2-(4- fluorobenzyloxy)-N- (3-(4-(N-(4-hydroxy- 3-methoxy- phenyl)sulfa- moyl)phenyl)pro- pyl)acetamide | (DMSO-d₆) δ (ppm) ¹H: 9.66 (s, 1H), 8.83 (s, 1H), 7.85 (t, J=5.9Hz, 1H), 7.56 (d, J=8.4Hz, 2H), 7.41 (dd, J=8.8 and 5.7Hz, 2H), 7.33 (d, J=8.4Hz, 2H), 7.16 (t, J=8.8Hz, 2H), 6.57-6.54 (m, 2H), 6.40 (dd, J=8.4 and 2.5Hz, 1H), 4.48 (s, 2H), 3.85 (s, 2H), | |
| # 3.58 (s, 3H), 3.07 (q, J=6.3Hz, 2H), 2.58 (t, J=7.6Hz, 2H), 1.69 (qi, J=7.2Hz, 2H). LRMS (ESI): 502.1 (calc) 503.0 (MH)+ (found) | 321 Ex 540a | | | | |

### EXAMPLE 541

### 2-(4-fluorobenzyloxy)-N-(3-(4-(N-(3,4,5-trimethoxyphenyl)sulfamoyl)phenyl)prop-2-ynyl)acetamide (793b)

Compound 793b was prepared according to the procedures described for compound 793a, example 540a, scheme 321, steps 1 and 2, replacing 1,4-benzodioxan-6-amine with 3,4,5-trimethoxyaniline in step 1.

(CDCl₃) δ (ppm) ¹H: 7.68 (d,2H,J=8.6 Hz), 7.46 (d,2H,J=8.4 Hz), 7.29-7.33 (m,2H), 7.04-7.08 (m,2H), 6.81 (s,1H), 6.34 (s,1H), 6.26 (s,1H), 4.55 (s,2H), 4.32 (d,2H,J=5.6 Hz), 4.11 (d,2H,J=7.2 Hz), 4.01 (s,2H), 3.78 (s,3H), 3.74 (s,6H).

LRMS (ESI): calc 542.15; found 541.0 (M−H)⁻

Examples 541c-f, compounds 793c-f were prepared as described for compound 793b Example 541, scheme 321. Characterization data are presented in Table 42.

TABLE 42

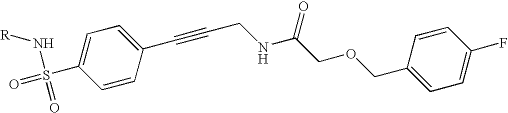

| Ex | Cpd | R | Name | Characterization | Scheme |
|---|---|---|---|---|---|
| 541c | 793c | 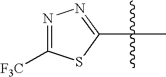 | 2-(4- fluorobenzyloxy)-N- (3-(4-(N-(5- (trifluoromethyl)- 1,3,4-thiadiazol-2- yl)sulfa- moyl)phenyl)prop- 2-ynyl)acetamide | (DMSO-d₆) δ (ppm) ¹H: 8.40 (t, J=5.7Hz, 1H), 7.77 (d, J=8.2Hz, 2H), 7.55 (d, J=8.2Hz, 2H), 7.42 (dd, J=8.8 and 5.7Hz, 2H), 7.17 (t, J=8.8Hz, 2H), 4.51 (s, 2H), 4.16 (d, J=5.7Hz, 2H), 3.94 (s, 2H). LRMS (ESI): (calc) 528.0; (found) 528.9 (MH)⁺ | 321 Ex 541 |
| 541d | 793d | 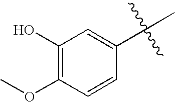 | 2-(4- fluorobenzyloxy)-N- (3-(4-(N-(3-hydroxy- 4-methoxy- phenyl)sulfa- moyl)phenyl)prop-2- ynyl)acetamide | (DMSO-d₆) δ (ppm) ¹H: 9.85 (s, 1H), 9.11 (s, 1H, 8.39 (t, J=5.9Hz, 1H), 7.61 (d, J=8.6Hz, 2H), 7.52 (d, J=8.6Hz, 2H), 7.42 (dd, J=8.8 and 5.7Hz, 2H), 7.17 (t, J=9.0Hz, 2H), 6.71 (d, J=8.8Hz, 1H), 6.52 (d, J=2.7Hz, 1H), 6.37 (dd, J=8.8 and 2.7Hz, 1H), | |
| # 4.50 (s, 2H), 4.14 (d, J=5.9Hz, 2H), 3.93 (s, 2H), 3.64 (s, 3H). LRMS (ESI): 498.1 (calc) 497.0 (M−) (found) | 321 Ex 541 | | | | |
| 541e | 793e | 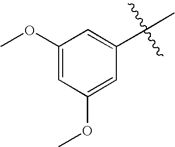 | N-(3-(4-(N-(3,5- dimethoxyphenyl)sul- famoyl)phenyl) prop-2-ynyl)-2-(4- fluorobenzyl- oxy)acetamide | (CDCl₃) δ (ppm) ¹H: 7.73 (d, 2H, J=8.4Hz), 7.45 (d, 2H, J=8.4Hz), 7.33-7.29 (m, 2H), 7.04-7.08 (m, 2H), 6.81 (s, 1H), 6.5 (s, 1H), 6.20 (m, 3H), 4.55 (s, 2H), 4.32 (d, 2H, J=5.6Hz), 4.01 (s, 6H) LRMS (ESI): (calc) 512.1; (found) 511.0 (M−H)⁻. | 321 Ex 541 |
| 541f | 793f | 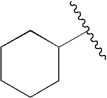 | N-(3-(4-(N- cyclohexylfulfamoyl) phenyl)prop-2-ynyl)- 2- (4-fluorobenzyl- oxy)acetamide | (MEOD-d₄) δ (ppm) ¹H: 7.81 (m, 2H), 7.56 (d, 2H, J=8.4Hz), 7.40-7.44 (m, 2H), 7.05-7.10 (m, 2H), 4.6 (s, 2H), 4.26 (s, 2H), 3.99 (s, 2H), 3.00 (s, 1H), 1.64 (d, 4H, J=9.8Hz), 1.5 (s, 1H), 1.13-1.22 (s, 5H). LRMS (ESI): (calc) 458.17; (found) 457.0 (M−H)− | 321 Ex 541 |

### EXAMPLE 542

### (Z)-2-(4-fluorobenzyloxy)-N-(3-(4-(N-(3,4,5-trimethoxyphenyl)sulfamoyl)phenyl)allyl)acetamide (795)

Compound 793b (226 mg, 04.44 mmol, example 541, scheme 321) in methanol (2.2 mL) and 10% palladium on charcoal (22 mg) was hydrogenated under 1 atm of H₂ for 16 h at room temperature. The residue was purified by silica gel column chromatography with gradient of ethyl acetate (40-60%) in hexane to give 795 (148 mg, 62%).

(DMSO-d₆) δ (ppm) ¹H: 10.14 (s,1H), 8.16 (t,1H,J=6.0 Hz), 7.74 (d,2H,J=8.4 Hz), 7.48 (d,2H,J=8.4 Hz), 7.38-7.42 (m,2H), 7.12-7.18 (m,2H), 6.47 (d,2H,J=12 Hz), 6,36 (s,2H), 5.68-5.74 (m,1H), 4.48 (s,2H), 3.97-4.01 (m,2H), 3.87 (s,2H), 3.62(s,6H), 3.53 (s,3H). LRMS (ESI): (calc) 544.2; (found) 543.0(M−H)⁻.

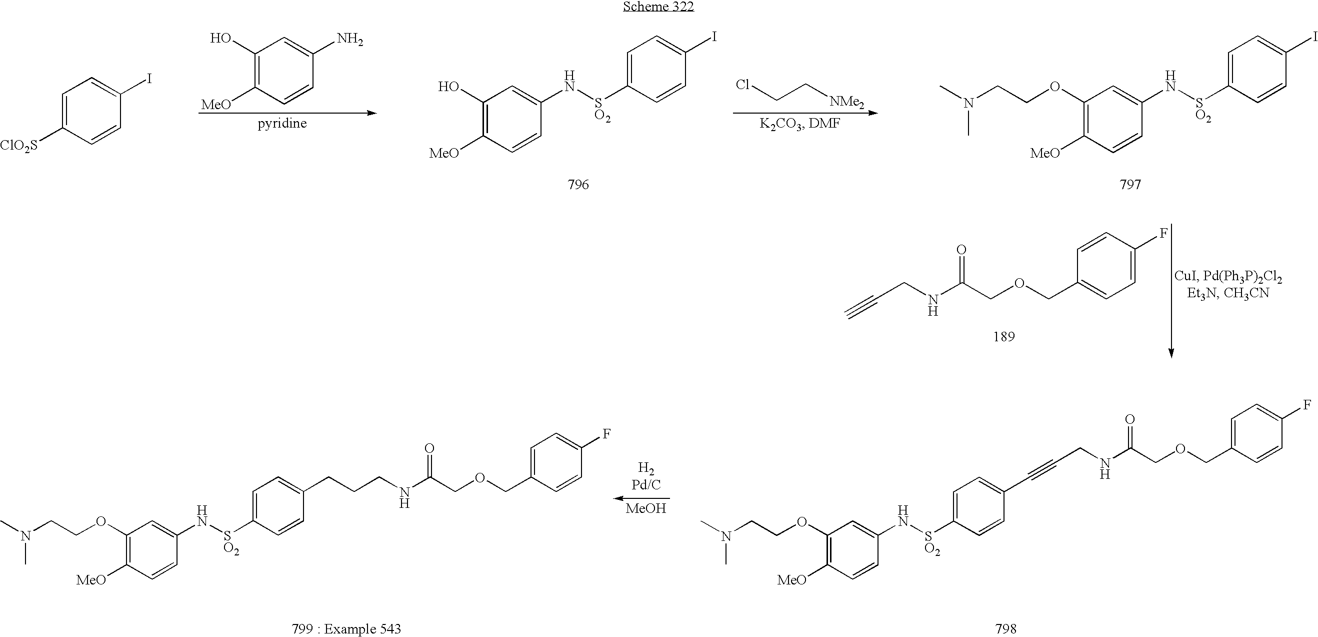

### EXAMPLE 543

### N-(3-(4-(N-(3-(2-(dimethylamino)ethoxy)-4-methoxyphenyl)sulfamoyl)phenyl)propyl)-2-(4-fluorobenzyloxy)acetamide (799)

### Step 1: N-(3-hydroxy-4-methoxyphenyl)-4-iodobenzenesulfonamide (796)

To a stirred solution of 5-amino-2-methoxyphenol (460 mg, 3.31 mmol) in pyridine (10 mL) was added 4-iodobenzenesulfonyl chloride (1.00 g, 3.31 mmol) as described for compound 792a, example 540a, scheme 321, step 1. The crude material was purified by silica gel column chromatography with gradient of ethyl acetate (20-40%) in hexane to give 796 (1.0 g, 75%).

(CDCl₃) δ (ppm) ¹H: 7.78 (d, J=8.6 Hz, 1H), 7.42 (d, J=8.6 Hz, 2H), 6.71 (d, J=8.6 Hz, 1H), 6.64 (d, J=2.5 Hz, 1H), 6.56 (dd, J=8.6 and 2.7 Hz, 1H), 6.51 (s, 1H), 5.77 (s, 1H), 3.85 (s, 3H). LRMS (ESI): (calc) 405.0; (found) 403.8 (M−H)⁻.

### Step 2: N-(3-(2-(dimethylamino)ethoxy)-4-methoxyphenyl)-4-iodobenzenesulfonamide (797)

To a stirred solution of 796 (250 mg, 0.62 mmol) in dimethylformamide (4.1 mL) was added potassium carbonate (256 mg, 1.85 mmol), and 2-(dimethylamino)ethyl chloride hydrochloride (98 mg, 0.68 mmol). The mixture was stirred for 3.5 h at 60° C. Brine was added and the aqueous phase was extracted with EtOAc, and the organic extract was dried (MgSO₄), filtered, and evaporated. The residue was purified by silica gel column chromatography with gradient of methanol (0-5%) in dichloromethane to give 797 (123 mg, 42%).

(DMSO-d₆) δ (ppm) ¹H: 9.21 (s, 1H), 7.94 (d, J=8.4 Hz, 2H), 7.34 (d, J=8.6 Hz, 2H), 6.82 (d, J=8.6 Hz, 1H), 6.46 (d, J=2.5 Hz, 1H), 6.38 (dd, J=8.4 and 2.5 Hz, 1H), 3.72 (s, 3H), 3.51 (t, J=6.8 Hz, 2H), 2.18 (t, J=6.8 Hz, 2H), 2.07 (s, 6H). LRMS (ESI): (calc) 476.0; (found) 476.9 (MH)⁺.

### Step 3: N-(3-(4-(N-(3-(2-(dimethylamino)ethoxy)-4-methoxyphenyl)sulfamoyl)phenyl)-prop-2-ynyl)-2-(4-fluorobenzyloxy)acetamide (798)

Compound 797 (123 mg, 0.26 mmol) in acetonitrile (1 mL) was added 2-(4-fluorobenzyloxy)-N-(prop-2-ynyl)acetamide (69 mg, 0.31 mmol), triethylamine (0.05 mL, 0.39 mmol), dichlorobis(triphenylphosphine)palladium(II) (9 mg, 0.01 mmol), and copper(I) iodide (5 mg, 0.03 mmol) as described for compound 793b, example 541, scheme 321. The material was purified by silica gel column chromatography with gradient of methanol (0-5%) in DCM to give 798 (100 mg, 68%).

(DMSO-d₆) δ (ppm) ¹H: 9.20 (s, 1H), 8.42 (t, J=6.1 Hz, 1H), 7.58-7.53 (m, 4H), 7.43 (dd, J=8.4 and 5.7 Hz, 2H), 7.17 (t, J=8.8 Hz, 2H), 6.81 (d, J=8.8 Hz, 1H), 6.42 (d, J=2.5 Hz, 1H), 6.34 (dd, J=8.4 and 2.5 Hz, 1H), 4.52 (s, 2H), 4.17 (d, J=5.9 Hz, 2H), 3.95 (s, 2H), 3.72 (s, 3H), 3.55-3.51 (m, 2H), 2.28-2.03 (m, 8H). LRMS (ESI): (calc) 569.2; (found) 570.0 (MH)⁺.

### Step 4: N-(3-(4-(N-(3-(2-(dimethylamino)ethoxy)-4-methoxyphenyl)sulfamoyl)phenyl)-propyl)-2-(4-fluorobenzyloxy)acetamide 799

To a stirred solution of 798 (80 mg, 0.14 mmol) in methanol (1.0 mL) was added 10% palladium on charcoal (16 mg). The mixture was stirred under 1 atm of H₂ for 16 h. The material was purified by silica gel column chromatography with a gradient of MeOH (0-10%) in DCM to give 799 (13 mg, 57%).

(DMSO-d₆) δ (ppm) ¹H: 9.17 (s, 1H), 7.89 (t, J=5.7 Hz, 1H), 7.49 (d, J=8.6 Hz, 2H), 7.43-7.37 (m, 4H), 7.17 (t, J=9.0 Hz, 2H), 6.80 (d, J=8.6 Hz, 1H), 6.45 (d, J=2.5 Hz, 1H), 6.34 (dd, J=8.6 and 2.5 Hz, 1H), 4.49 (s, 2H), 3.86 (s, 2H), 3.72 (s, 3H), 3.49 (t, J=7.0 Hz, 2H), 3.10 (q, J=6.3 Hz, 2H), 2.64 (t, J=8.0 Hz, 2H), 2.18 (t, J=6.8 Hz, 2H), 1.73 (qi, J=7.4 Hz, 2H). LRMS (ESI): (calc) 573.2; (found) 574.1 (MH)⁺.

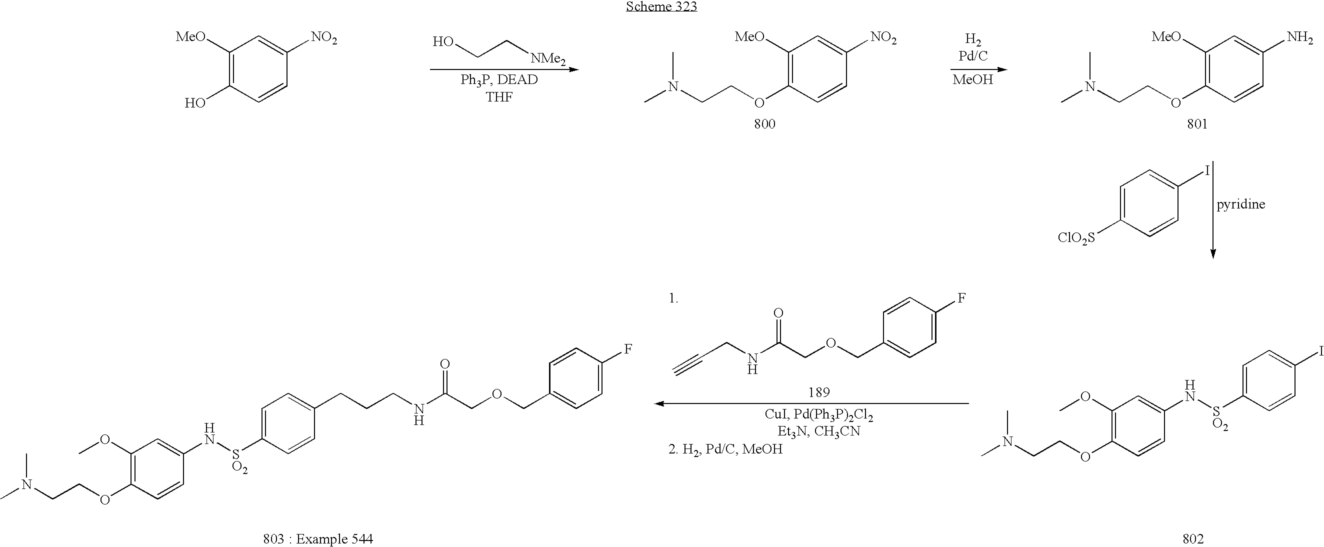

### EXAMPLE 544

### N-(3-(4-(N-(4-(2-(dimethylamino)ethoxy)-3-methoxyphenyl)sulfamoyl)phenyl)propyl)-2-(4-fluorobenzyloxy)acetamide (803)

### Step 1: 2-(2-methoxy-4-nitrophenoxy)-N,N-dimethylethanamine (800)

To a stirred solution of 4-nitroguaiacol (750 mg, 4.32 mmol) in THF (39 mL) was added N,N-dimethylethanolamine (0.40 mL, 3.92 mmol), triphenylphosphine (1.34 g, 5.10 mmol) and diethylazodicarboxylate (0.73 mL, 4.71 mmol). The mixture was stirred for 16 h at room temperature. Saturated sodium carbonate (aq.) was added and the mixture was extracted with EtOAc. The organic extract was dried (MgSO₄), filtered, and concentrated and the residue was purified by silica gel column chromatography with gradient of MeOH (0-10%) in DCM to give 800 (310 mg, 33%).

(DMSO-d₆) δ (ppm) ¹H: 7.87 (dd, J=8.8 and 2.5 Hz, 1H), 7.71 (d, J=2.7 Hz, 1H), 7.18 (d, J=9.0 Hz, 1H), 4.16 (t, J=5.9 Hz, 2H), 3.86 (s, 3H), 2.64 (t, J=5.7 Hz, 2H), 2.19 (s, 6H).

### Step 2: 4-(2-(dimethylamino)ethoxy)-3-methoxybenzenamine (801)

To a stirred solution of 800 (310 mg, 1.29 mmol) in methanol (6 mL) was added 10% palladium on charcoal (31 mg). The mixture was stirred under 1 atm of H₂ for 3.5 h at room temperature. The material was purified by silica gel column chromatography with gradient of methanol (0-20%) in dichloromethane to give 801 (237 mg, 87%).

(DMSO-d₆) δ (ppm) ¹H: 6.62 (d, J=8.4 Hz, 1H), 6.23 (d, J=2.5 Hz, 1H), 6.01 (dd, J=8.4 and 2.5 Hz, 1H), 4.67 (bs, 2H), 3.82 (t, J=6.1 Hz, 2H), 3.64 (s, 3H), 2.50 (t, J=6.1 Hz, 2H), 2.16 (s, 6H). LRMS (ESI): (calc) 210.1; (found) 211.1 (MH)+.

### Step 3: N-(4-(2-(dimethylamino)ethoxy)-3-methoxyphenyl)-4-iodobenzenesulfonamide (802)

To a stirred solution of 801 (237 mg, 1.13 mmol) in pyridine (3.5 mL) was added 4-iodobenzenesulfonyl chloride (341 mg, 1.13 mmol) as described for compound 792a, example 540a, scheme 321, step 1. The material was purified by silica gel column chromatography with gradient of methanol (0-20%) in dichloromethane to give 802 (410 mg, 76%).

(DMSO-d₆) δ (ppm) ¹H: 9.97 (bs, 1H), 7.91 (d, J=8.6 Hz, 2H), 7.42 (d, J=8.6 Hz, 2H), 6.80 (d, J=8.6 Hz, 1H), 6.64 (d, J=2.3 Hz, 1H), 6.48 (dd, J=8.6 and 2.5 Hz, 1H), 3.89 (t, J=5.9 Hz, 2H), 3.61 (s, 3H), 2.54 (t, J=5.9 Hz, 2H), 2.16 (s, 6H). LRMS (ESI): (calc) 476.0; (found) 476.9 (MH)⁺.

### Step 4: N-(3-(4-(N-(4-(2-(dimethylamino)ethoxy)-3-methoxyphenyl)sulfamoyl)phenyl)-propyl)-2-(4-fluorobenzyloxy)acetamide (803)

To a stirred solution of 802 (196 mg, 0.41 mmol) in acetonitrile (1.2 mL) was added 2-(4-fluorobenzyloxy)-N-(prop-2-ynyl)acetamide (100 mg, 0.45 mmol); triethylamine (0.09 mL, 0.62 mmol), dichlorobis(triphenylphosphine)palladium(II) (14 mg, 0.02 mmol), and copper(I) iodide (8 mg, 0.04 mmol) as described for example 541, compound 793b, scheme 321. The material was purified by silica gel column chromatography with gradient of methanol (0-20%) in DCM to give the alkyne product (185 mg, 79%).

(DMSO-d₆) δ (ppm) ¹H: 9.96 (s, 1H), 8.39 (t, J=5.9 Hz, 1H), 7.63 (d, J=8.4 Hz, 2H), 7.53 (d, J=8.4 Hz, 2H), 7.42 (dd, J=8.4 and 5.5 Hz, 2H), 7.16 (t, J=9.0 Hz, 2H), 6.79 (d, J=8.6 Hz, 1H), 6.63 (d, J=2.5 Hz, 1H), 6.46 (dd, J=8.6 and 2.5 Hz, 1H), 4.50 (s, 2H), 4.14 (d, J=5.9 Hz, 2H), 3.93 (s, 2H), 3.89 (t, J=5.9 Hz, 2H), 3.60 (s, 3H), 2.53 (t, J=5.9 Hz, 2H), 2.15 (s, 6H). LRMS (ESI): (calc) 569.2; (found) 570.1 (MH)⁺.

Reduction of the alkyne above (148 mg, 0.26 mmol) in methanol (1.0 mL) using 10% palladium on charcoal (15 mg).under 1 atm of H₂ for 16 h at room temperature gave 803 (136 mg, 91%).

(DMSO-d₆) δ (ppm) ¹H: 9.85 (s, 1H), 7.85 (t, J=5.9 Hz, 1H), 7.59 (d, J=8.4 Hz, 2H), 7.40 (dd, J=8.4 and 5.7 Hz, 2H), 7.34 (d, J=8.2 Hz, 2H), 7.16 (t, J=8.8 Hz, 2H), 6.78 (t, J=8.6 Hz, 1H), 6.63 (d, J=2.5 Hz, 1H), 6.50 (dd, J=8.6 and 2.5 Hz, 1H), 4.48 (s, 2H), 3.88 (t, J=5.9 Hz, 2H), 3.84 (s, 2H), 3.59 (s, 3H), 3.07 (q, J=6.5 Hz, 2H), 2.58 (t, J=7.6 Hz, 2H), 2.53 (t, J=5.9 Hz, 2H), 2.15 (s, 6H), 1.68 (qi, J=7.6 Hz, 2H). LRMS (ESI): (calc) 573.2; (found) 574.1 (MH)+.

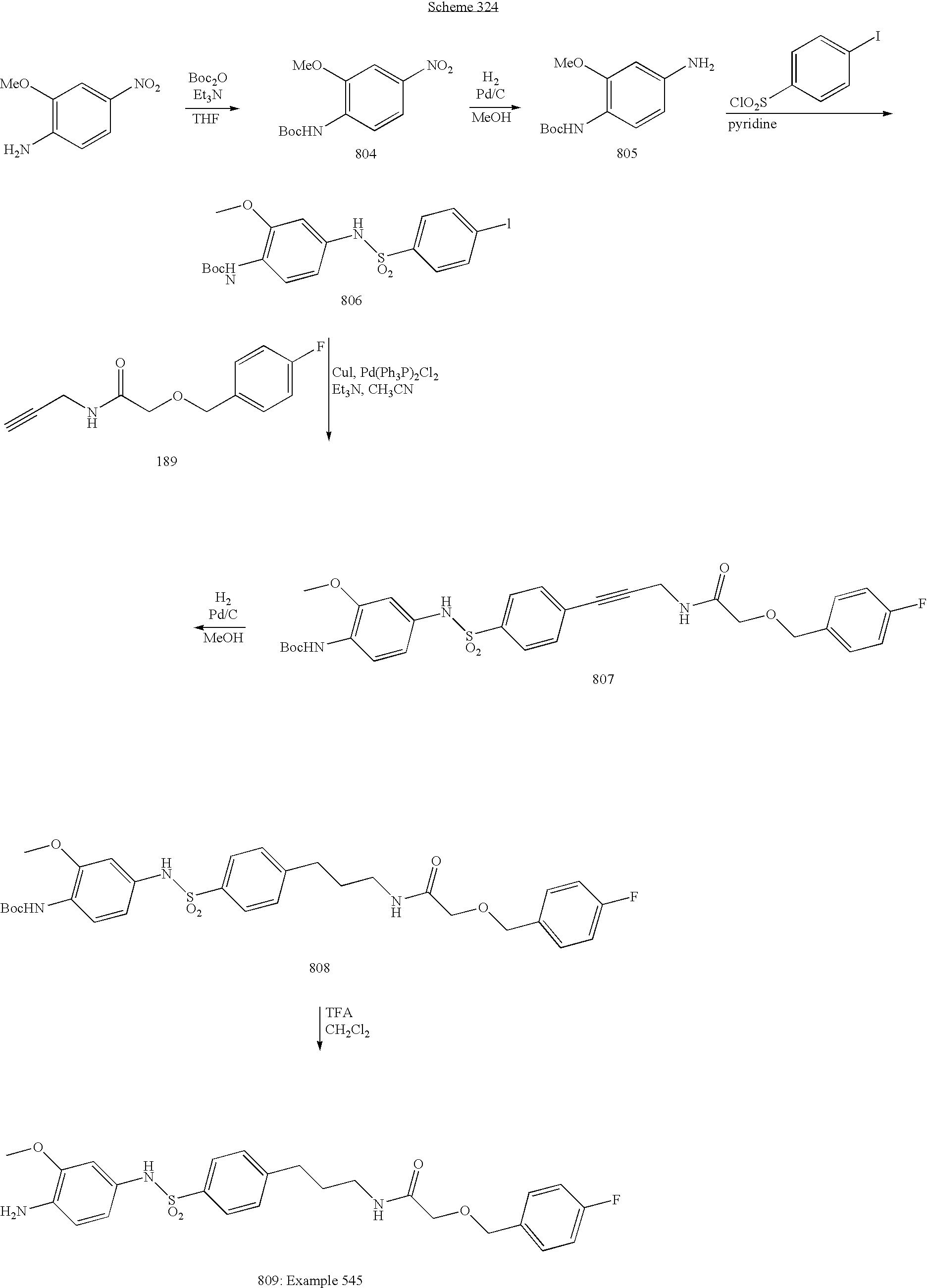

### EXAMPLE 545

### N-(3-(4-(N-(4-amino-3-methoxyphenyl)sulfamoyl)phenyl)propyl)-2-(4-fluorobenzyloxy)acetamide (809)

### Step 1: tert-butyl 2-methoxy-4-nitrophenylcarbamate (804)

To a stirred solution of 2-methoxy-4-nitroaniline (2.5 g, 15 mmol) in THF (75 mL) was added di-tert-butyl dicarbonate (3.89 g, 18 mmol). The mixture was stirred for 16 h at reflux. Triethylamine (3 mL, 30 mmol) was added, and then the mixture was stirred for 16 h at reflux. The mixture was concentrated and the residue was purified by silica gel column chromatography with 30% ethyl acetate in hexane to give 804 (987 mg, 25%).

LRMS (ESI): (calc) 268.1; (found) 269.0 (MH)⁺.

### Step 2: tert-butyl 4-amino-2-methoxyphenylcarbamate (805)

Compound 804 (805 mg, 3.0 mmol) in methanol (15 mL) and 10% palladium on charcoal (80 mg) was hydrogenated under 1 atm of H₂ for 16 h at room temperature. The material was purified by silica gel column chromatography with gradient of methanol (0-10%) in dichloromethane to give 805 (215 mg, 30%).

(DMSO-d₆) δ (ppm) ¹H: 7.52 (s, 1H), 7.04-6.98 (m, 1H), 6.20 (d, J=2.3 Hz, 1H), 6.05 (dd, J=8.4 and 2.3 Hz, 1H), 5.74 (s, 2H), 3.65 (s, 3H), 1.38 (s, 9H). LRMS (ESI): (calc) 238.1; (found)183.0 (MH-tBu)+.

### Step 3: tert-butyl 4-(4-iodophenylsulfonamido)-2-methoxyphenylcarbamate (806)

To a stirred solution of 805 (215 mg, 0.90 mmol) in pyridine (3 mL) was added 4-iodobenzenesulfonyl chloride (272 mg, 0.90 mmol) as described for compound 792a, example 540a, scheme 321, step 1. The material was purified by silica gel column chromatography with gradient of ethyl acetate (20-40%) in hexane to give 806 (387 mg, 85%).

(CDCl₃) δ (ppm) ¹H: 10.15 (s, 1H), 7.91 (d, J=8.4 Hz, 2H), 7.85 (s, 1H), 7.46 (d, J=8.6 Hz, 2H), 7.44-7.40 (m, 1H), 6.70 (d, J=2.3 Hz, 1H), 6.53 (dd, J=8.6 and 2.3 Hz, 1H), 3.67 (s, 3H), 1.39 (s, 9H). LRMS (ESI): (calc) 504.0; (found) 526.8 (MNa)⁺.

### Step 4: tert-butyl 4-(4-(3-(2-(4-fluorobenzyloxy)acetamido)prop-1-ynyl)phenylsulfonamido)-2-methoxyphenylcarbamate (807)

Compound 806 (158 mg, 0.31 mmol) in acetonitrile (1.0 mL) was reacted with 2-(4-fluorobenzyloxy)-N-(prop-2-ynyl)acetamide (82 mg, 0.37 mmol), triethylamine (0.07 mL, 0.47 mmol), dichlorobis(triphenylphosphine)palladium(II) (11 mg, 0.02 mmol), and copper(I) iodide (6 mg, 0.03 mmol) as described for compound 793b, example 541, scheme 321. The material was purified by silica gel column chromatography with gradient of ethyl acetate (40-60%) in hexane to give 807 (107 mg, 57%).

(DMSO-d₆) δ (ppm) ¹H: 10.14 (s, 1H), 8.38 (t, J=5.9 Hz, 1H), 7.85 (s, 1H), 7.67 (d, J=8.6 Hz, 2H), 7.53 (d, J=8.6 Hz, 2H), 7.43-7.39 (m, 3H), 7.16 (t, J=9.0 Hz, 2H), 6.69 (d, J=2.3 Hz, 1H), 6.52 (dd, J=2.3 and 8.6 Hz, 1H), 4.50 (s, 2H), 4.14 (d, J=5.9 Hz, 2H), 3.93 (s, 2H), 3.65 (s, 3H), 1.39 (s, 9H). LRMS (ESI): (calc) 597.2; (found) 620.0 (MNa)⁺.

### Step 5: tert-butyl 4-(4-(3-(2-(4-fluorobenzyloxy)acetamido)propyl)phenylsulfonamido)-2-methoxyphenylcarbamate (808)

Compound 807 (97 mg, 0.16 mmol) in MeOH (1.0 mL) and 10% palladium on charcoal (10 mg) was hydrogenated under 1 atm of H₂ for 16 h at room temperature. The material was purified by silica gel column chromatography with gradient of ethyl acetate (40-80%) in hexane to give 808 (67 mg, 68%).

(DMSO-d₆) δ (ppm) ¹H, 10.04 (s, 1H), 7.84-7.83 (m, 2H), 7.63 (d, J=8.4 Hz, 2H), 7.42-7.36 (m, 3H), 7.35 (d, J=8.4 Hz, 2H), 7.16 (t, J=9.0 Hz, 2H), 6.70 (d, J=2.2 Hz, 1H), 6.55 (dd, J=8.4 and 2.2 Hz, 1H), 4.48 (s, 2H), 3.84 (s, 2H), 3.64 (s, 3H), 3.08 (q, J=6.5 Hz, 2H), 2.58 (t, J=7.6 Hz, 2H), 1.69 (qi, J=7.6 Hz, 2H), 1.38 (s, 9H). LRMS (ESI): (calc) 601.2; (found) 602.1 (MH)⁺.

### Step 6: N-(3-(4-(N-(4-amino-3-methoxyphenyl)sulfamoyl)phenyl)propyl)-2-(4-fluorobenzyloxy)acetamide (809)

To a stirred solution of 808 (58 mg, 0.10 mmol) in DCM (0.5 mL) was added trifluoroacetic acid (0.1 mL). The mixture was stirred for 1.25 h at room temperature. The solvent was evaporated, a solution of saturated sodium carbonate in water was added, and then the residue was extracted with DCM. The organic extract was dried (MgSO₄), filtered, and evaporated to give 809 (38 mg, 79%).

(DMSO-d₆) δ (ppm) ¹H, 9.44 (s,1H), 7.85 (t, J=5.7 Hz, 1H), 7.54 (d, J=8.4 Hz, 2H), 7.41 (dd, J=8.8 and 5.7 Hz, 2H), 7.32 (d, J=8.4 Hz, 2H), 7.16 (t, J=9.0 Hz, 2H), 6.42-6.38 (m, 2H), 6.31 (dd, J=8.2 and 2.2 Hz, 1H), 4.56 (s, 2H), 4.48 (s, 3H), 3.85 (s, 2H), 3.56 (s, 3H), 3.08 (q, J6.5 Hz, 2H), 2.58 (t, J=7.4 Hz, 2H), 1.69 (qi, J=7.0 Hz, 2H). LRMS (ESI): (calc) 501.2; (found) 502.1 (MH)⁺.

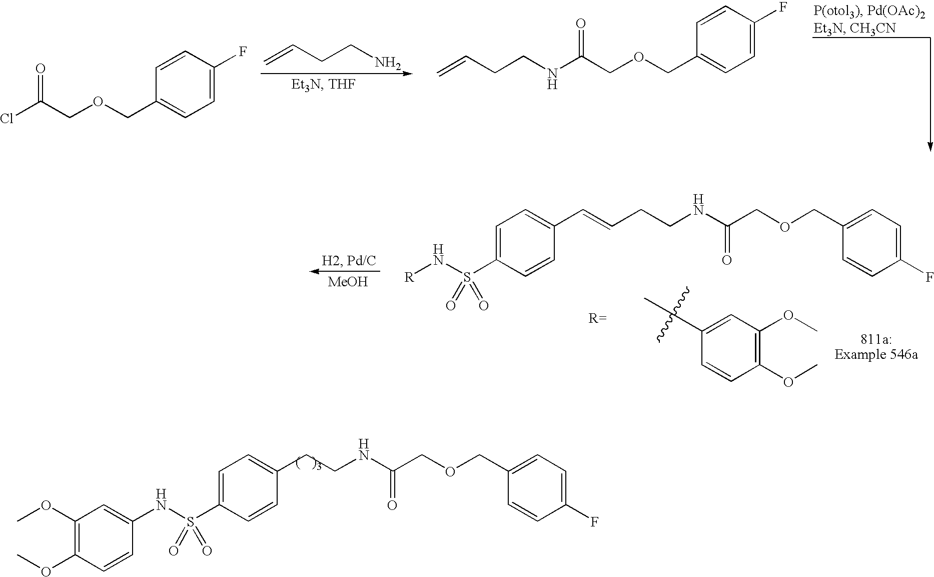

### EXAMPLE 546a

### (E)-N-(4-(4-(N-(3,4-dimethoxyphenyl)sulfamoyl)phenyl)but-3-enyl)-2-(4-fluorobenzyloxy)acetamide (811a)

### Step 1: 2-(4-fluorobenzyloxy)-N-(but-3-enyl)acetamide (810)

A solution of acid chloride 289 (5.43 mmol) in THF (4 mL) was added to 3-buten-1-amine hydrochloride (584 mg, 5.43 mmol) and triethylamine (2.3 mL, 16.3 mmol) in THF (18 mL) at 0° C. as described for compound 724, example 508a, step 1,scheme 303. The material was purified by silica gel column chromatography with ethyl acetate (40%) in hexane to give 810 (575 mg, 45%).

(DMSO-d₆) δ (ppm) ¹H: 7.79 (bs, 1H), 7.40 (dd, J=9.1 and 5.5 Hz, 2H), 7.17 (t, J=8.8 Hz, 2H), 5.79-5.69 (m, 1H), 5.06-4.96 (m, 2H), 4.48 (s, 2H), 3.85 (s, 2H), 3.15 (q, J=7.0 Hz, 2H), 2.16 (q, J=7.0 Hz, 2H). LRMS (ESI): (calc) 237.1; (found) 238.0 (MH)⁺.

### Step 2: (E)-N-(4-(4-(N-(3,4-dimethoxyphenyl)sulfamoyl)phenyl)but-3-enyl)-2-(4-fluorobenzyloxy)acetamide (811a)

To a stirred solution of N-(3,4-dimethoxyphenyl)-4-iodobenzenesulfonamide (1.01 mg, 2.42 mmol) in acetonitrile (6 mL) was added tri-otolylphosphine (74.8 mg, 0.24 mmol), palladium(II) acetate (27 mg, 0.12 mmol), 810a (690 mg, 2.91 mmol), and triethylamine (0.63 mL, 4.84 mmol) as described for compound 761a, example 529, step 1, scheme 314. The material was purified by silica gel column chromatography with gradient of ethyl acetate (0-90%) in hexane to give 811a (211 mg, 16%) as white solid.

(DMSO-d₆) δ (ppm) ¹H, 9.86 (s,1H), 7.88 (broad triplet, 1H), 7.59 (d, J=8.8 Hz, 2H), 7.48 (d, J=8.8 Hz, 2H), 7.35-7.31 (m, 2H), 7.13-7.09 (m, 2H), 6.74 (d, J=8.8 Hz, 1H), 6.65 (d, J=2.8 Hz, 1H), 6.50 (dd, J=8.4, 2.4 Hz, 1H), 6.46-6.34 (m, 2H), 4.45 (s, 2H), 3.84 (s, 2H), 3.62 (s, 3H), 3.59 (s, 3H), 3.25-3.21 (m, 2H), 2.36-2.31 (m, 2H).

LRMS (ESI): (calc) 528.6; (found) 529.0 (MH)⁺.

### EXAMPLE 546b

### (E)-N-(4-(4-(N-(2,3-dihydrobenzo[b][1,4]dioxin-6-yl)sulfamoyl)phenyl)but-3-enyl)-2-(4-fluorobenzyloxy)acetamide (811b)

Compound 811b, Example 646b, was obtained in 20% overall yield following the procedures desribed for compound 811a, Example 546a, scheme 325, replacing N-(3,4-dimethoxyphenyl)-4-iodobenzenesulfonamide in step 2, with N-(2,3-dihydrobenzo[b][1,4]dioxin-6-yl)-4-iodobenzenesulfonamide.

(MEOD-d₄) δ(ppm) ¹H: 7.60 (d, 2H, J=8.4 Hz), 7.43 (d, 2H, J=8.6 Hz), 7.30 (dd, 2H, J1=5.4 Hz,J2=3.1 Hz), 7.00 (t, 2H, J=8.8 Hz), 6.62 (d, 1H, J=8.6 Hz), 6.57 (d, 1H, J=2.5 Hz), 6.45-6.49 (m, 2H), 6.36 (dt, 2H, J1=6.6 Hz,J2=15.8 Hz), 4.50 (s, 2H), 4.13 (s, 4H), 3.90 (s, 2H), 3.38 (t, 2H, J=6.8 Hz), 2.44 (q, 2H, J=6.4 Hz). LRMS (ESI): (calc) 526.1; (found) 525.0 (M−H)⁻.

### EXAMPLE 547

### N-(4-(4-(N-(3,4-dimethoxyphenyl)sulfamoyl)phenyl)butyl)-2-(4-fluorobenzyloxy)acetamide (812)

Compound 811a (79 mg, 0.165 mmol) in MeOH (1.0 mL) and 10% palladium on charcoal (16 mg) was hydrogenated under 1 atm of H₂ for 16 h at room temperature. The material was purified by silica gel column chromatography with gradient of ethyl acetate (40-80%) in hexane to give 812 (20 mg, 25%) as clear oil.

(MEOD-d₄) δ(ppm) ¹H: 7.59 (d, J=8.4 Hz, 2H), 7.39-7.36 (m, 2H), 7.28 (d, J=8.4 Hz, 2H), 7.08-7.03 (m, 2H), 6.45 (d, J=8.8 Hz, 1H), 6.65 (d, J=2.4 Hz, 1H), 6.56 (dd, J=8.6, 2.4 Hz, 1H), 4.54 (s, 2H), 3.91 (s, 2H), 3.72 (s, 3H), 3.67 (s, 2H), 3.22 (t, J=7.2 Hz, 2H), 2.66 (t, J=7.6 Hz, 2H), 1.63-1.56 (m, 2H), 1.52-1.45 (m, 2H).

LRMS: (calc) 530.6; (found) 531.0 (MH)⁺.

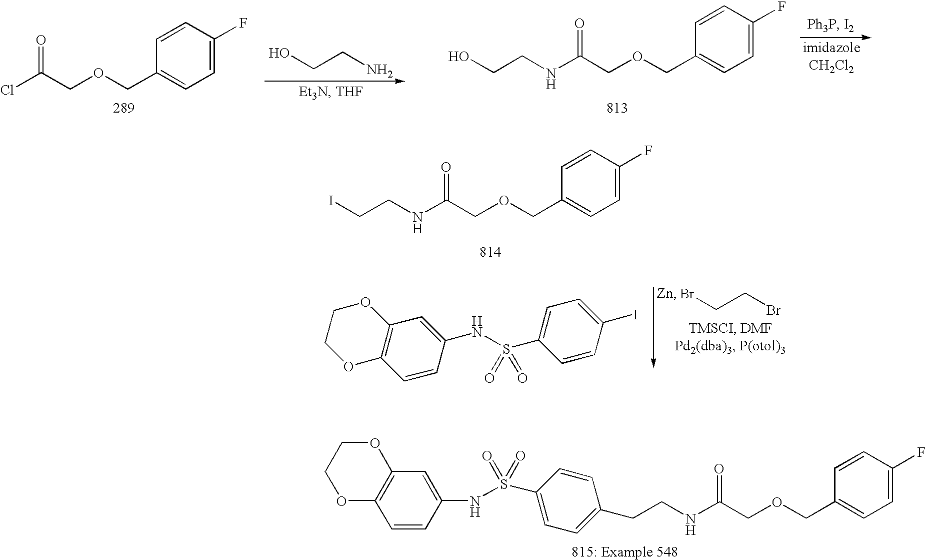

### EXAMPLE 548

### N-(4-(N-(2,3-dihydrobenzo[b][1,4]dioxin-6-yl)sulfamoyl)phenethyl)-2-(4-fluorobenzyloxy)acetamide (815)

### Step 1: 2-(4-fluorobenzyloxy)-N-(2-hydroxyethyl)acetamide (813)

A solution of acid chloride 289 (5.43 mmol) in THF (4 mL) was added to ethanolamine (0.33 mL, 5.43 mmol) and triethylamine (1.5 mL, 10.9 mmol) in THF (18 mL) at 0° C. as described for compound 724, example 508a, step 1, scheme 303. The material was purified by silica gel column chromatography with gradient of EtOAc (60-100%) in hexanes to give 813 (330 mg, 27%).

(DMSO-d₆) δ (ppm) ¹H: 7.69 (t, J=5.1 Hz, 1H), 7.41 (dd, J=8.8 and 5.7 Hz, 2H), 7.17 (t, J=9.0 Hz, 2H), 4.70 (t, J=5.5 Hz, 1H), 4.49 (s, 2H), 3.86 (s, 2H), 3.39 (q, J=5.7 Hz, 2H), 3.15 (q, J=6.1 Hz, 2H). LRMS (ESI): (calc) 227.1; (found).228.0 (MH)+.

### Step 2: 2-(4-fluorobenzyloxy)-N-(2-iodoethyl)acetamide (814)

To a stirred solution of triphenylphosphine (381 mg, 1.45 mmol) in DCM (5 mL) was added imidazole (100 mg; 1.45 mmol) followed by iodine (406 mg, 1.60 mmol) and a solution of 813 (330 mg, 1.45 mmol) in DCM (2 mL) was added. The reaction was stirred at room temperature for 16 h. The solvent was evaporated and the residue was purified by silica gel column chromatography with gradient of EtOAc (20-40%) in hexane to give 814 (395 mg, 81%).

(DMSO-d₆) δ (ppm) ¹H, 8.11 (t, J=5.5 Hz,1H), 7.42 (dd, J=8.8 and 5.7 Hz, 2H), 7.18 (t, J=9.0 Hz, 2H), 4.51 (s, 2H), 3.88 (s, 2H), 3.43 (q, J=6.1 Hz, 2H), 3.21 (t, J=6.8 Hz, 2H). LRMS (ESI): (calc) 337.0; (found).359.9 (MNa)⁺.

### Step 3: N-(4-(N-(2,3-dihydrobenzo[b][1,4]dioxin-6-yl)sulfamoyl)phenethyl)-2-(4-fluorobenzyloxy)acetamide (815)

Zinc dust (294 mg, 4.50 mmol) in a round bottom flask was heated with a heat-gun for 30 sec under nitrogen. After cooling to room temperature, dimethylformamide (0.5 mL) was added, followed by 1,2-dibromoethane (30 uL, 0.36 mmol) according to the method of Hunter, C.; Jackson, R.; Rami, H. (J. Chem. Soc. Perkin Trans 1 2000, 219-223). The reaction was stirred at 80° C. for 1 min. After cooling to room temperature, chlorotrimethylsilane (20 uL, 0.25 mmol) was added and the reaction was stirred for 30 min. A solution of 814 (253 mg, 0.75 mmol) in dimethylformamide (0.5 mL) was added, and the mixture was stirred for another 30 min. Then tris(dibenzylideneacetone)dipalladium (23 mg, 0.03 mmol) was added, followed by tri-o-tolylphosphine (30 mg, 0.10 mmol) and N-(2,3-dihydrobenzo[b][1,4]dioxin-6-yl)-4-iodobenzenesulfonamide (253 mg, 0.75 mmol). The reaction was stirred for 16 h at room temperature. The mixture was diluted with ethyl acetate and washed with brine twice. The organic extract was dried (MgSO₄), filtered, and evaporated, and the crude was purified by silica gel column chromatography with gradient of ethyl acetate (60-80%) in hexane to give 815 (130 mg, 35%).

(DMSO-d₆) δ (ppm) ¹H, 9.92 (s,1H), 7.85 (t, J=5.7 Hz, 1H), 7.61 (d, J=8.4 Hz, 2H), 7.37-7.33 (m, 4H), 7.16 (t, J=9.0 Hz, 2H), 6.65 (d, J=8.8 Hz, 1H), 6.55 (d, J=2.5 Hz, 1H), 6.48 (dd, J=8.8 and 2.5 Hz, 1H), 4.42 (s, 2H), 4.13-4.10 (m, 4H), 3.81 (s, 2H), 3.32 (q, J=6.8 Hz, 2H), 2.78 (t, J=7.2 Hz, 2H). LRMS (ESI): (calc) 500.1; (found).499.1 (M−H)⁻.

Example 548a describe the preparation of compound 815a using the same procedures as described for compound 815 in Example 548, scheme 326, replacing N-(2,3-dihydrobenzo[b][1,4]dioxin-6-yl)-4-iodobenzenesulfonamide in step 3 with N-(3,5-dimethoxyphenyl)-4-iodobenzenesulfonamide. Characterization data is presented in Table 43.

Example 549a-c describe the preparation of compound 816a-c using the same procedures as described for compound 815 in Example 548, scheme 326, except using 4-aminobutan-1-ol in place of 2-aminoethanol in step 1, and replacing N-(2,3-dihydrobenzo[b][1,4]dioxin-6-yl)-4-iodobenzenesulfonamide in step 3 with with N-(3-fluoro-4-methoxyphenyl)-4-iodobenzenesulfonamide for compound 816b, and with 4-iodo-N-(3,4,5-trimethoxyphenyl)benzenesulfonamide for compound 816c.

TABLE 43

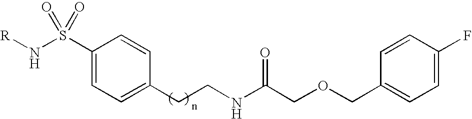

| Ex | Cpd | R | n | Name | Characterization | Scheme |
|---|---|---|---|---|---|---|
| 548a | 815a | 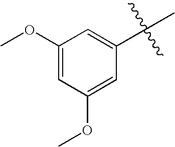 | 1 | N-(4-(N-(3,5- dimethoxyphenyl)sul- famoyl)phenethyl)-2- (4-fluoro- benzyloxy)acetamide | DMSO-d₆) δ (ppm) ¹H: 10.25 (s, 1H), 7.86 (t, J=5.9Hz, 1H), 7.69 (d, J=8.4Hz, 2H), 7.37 (d, J=8.4Hz, 2H), 7.36-7.33 (m, 2H), 7.16 (t, J=9.0Hz, 2H), 6.24 (d, J=2.2Hz, 2H), 6.12 (t, J=2.2Hz, 1H), 4.41 (s, 2H), 3.81 (s, 2H), 3.60 (s, 6H), 3.32 (q, | |
| # J=6.8Hz, 2H), 2.78 (t, J=7.0Hz, 2H). LRMS (ESI): (calc) 502.1; (found) 503.0 (MH)+. | 326 Step 1-3 Ex 548 | | | | | |
| 549a | 816a | 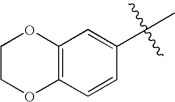 | 3 | N-(4-(4-(N-(2,3- dihydrobenzo[b][1,4]dioxin-6- yl)sulfamoyl)phenyl) butyl)-2-(4-fluoro- benzyloxy)acetamide | (DMSO-d₆) δ (ppm) ¹H: 9.90 (s, 1H), 7.80 (t, J=6.1Hz, 1H), 7.59 (d, J8.4Hz, 2H), 7.39 (dd, J=8.8 and 5.7Hz, 2H), 7.33 (d, J=8.6Hz, 2H), 7.15 (t, J=9.0Hz, 2H), 6.66 (d, J=8.8Hz, 1H), 6.55 (d, J=2.5Hz, 1H), 6.49 (dd, J=8.6 and 2.5Hz, 1H), | |
| # 4.47 (s, 2H), 4.14-4.10 (m, 4H), 3.84 (s, 2H), 3.08 (q, J=6.5Hz, 2H), 2.59 (t, J=7.4Hz, 2H), 1.50 (qi, J=8.0Hz, 2H), 1.38 (qi, J=7.0Hz, 2H). LRMS (ESI): (calc) 528.1; (found) 529.0 (MH)+ | 326 Step 1-3 Ex 548 | | | | | |
| 549b | 816b | 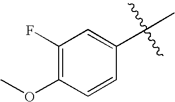 | 3 | N-(4-(4-(N-(3-fluoro- 4- methoxyphenyl)sulfa- moyl)phenyl)butyl)-2- (4-fluoro- benzyloxy)acetamide | (DMSO-d₆) δ (ppm) ¹H: 7.60 (d, J=8.6Hz, 2H), 7.38 (dd, J=9.0 and 5.5Hz, 2H), 7.30 (d, J=8.4Hz, 2H), 7.06 (t, J=8.8Hz, 2H), 6.91 (t, J=9.0Hz, 1H), 6.84 (dd, J=12.5 and 2.5Hz, 1H), 6.77-6.74 (m, 1H), 4.55 (s, 2H), 3.91 (s, 2H), 3.77 (s, 3H), | |
| # 3.23 (t, J=7.0Hz, 2H), 2.67 (t, J=7.4Hz, 2H), 1.65-1.57 (m, 2H), 1.54-1.46 (m, 2H). LRMS (ESI): (calc) 518.1; (found) 519.0 (MH)+ | 326 Step 1-3 Ex 548 | | | | | |
| 549c | 816c | 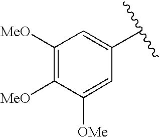 | 3 | 2-(4- fluorobenzyloxy)-N- (4-(4-(N-(3,4,5- trimethoxyphenyl)sul- famoyl)phenyl) butyl)acetamide | (DMSO-d₆) δ (ppm) ¹H: 10.04 (s, 1H), 7.79 (t, J=5.7Hz, 1H), 7.66 (d, J=8.4Hz, 2H), 7.40-7.34 (m, 4H), 7.15 (t, J=9.0Hz, 2H), 6.34 (s, 2H), 4.47 (s, 2H), 3.83 (s, 2H), 3.61 (s, 6H), 3.52 (s, 3H), 3.08 (q, J=6.3Hz, 2H), 2.60 (t, J=7.4Hz, 2H), 1.50 | |
| # (qi, J=7.8Hz, 2H), 1.37 (qi, J=7.6Hz, 2H). LRMS (ESI): (calc) 560.2; (found) 561.1 (MH)⁺ | 326 Step 1-3 Ex 548 | | | | | |

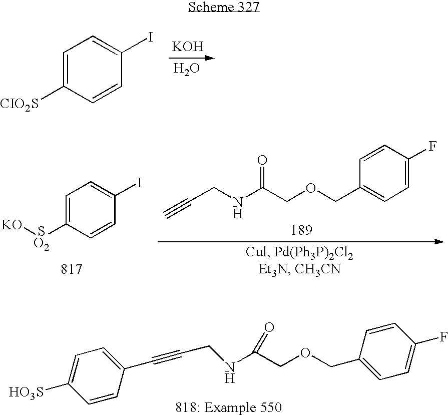

### EXAMPLE 550

### 4-(3-(2-(4-fluorobenzyloxy)acetamido)prop-1-ynyl)benzenesulfonic acid

### Step 1: potassium 4-iodobenzenesulfonate (817)

To a stirred solution of 4-iodobenzenesulfonyl chloride (500 mg, 1.65 mmol) in water (3.3 mL) was added potassium hydroxide (924 mg, 16.5 mmol). The mixture was stirred for 1.25 h at 80° C. The precipitate was filtered, and ried under reduced pressure to give 817 (257 mg, 48%).

### Step 2: 4-(3-(2-(4-fluorobenzyloxy)acetamido)prop-1-ynyl)benzenesulfonic acid (818)

Compound 818 was prepared following the procedure described for compound 793b, example 541, scheme 321.

(DMSO-d₆) d(ppm) ¹H, 8.39 (t, J=5.9 Hz, 1H), 7.54 (d, J=7.8 Hz, 2H), 7.43 (dd, J=8.8 and 5.7 Hz, 2H), 7.34 (d, J=8.0 Hz, 2H), 7.17 (t, J=9.0 Hz, 2H), 4.51 (s, 2H), 4.14 (d, J=5.9 Hz, 2H), 3.94 (s, 2H). LRMS (ESI): (calc) 377.0; (found) 375.9 (M−H)⁻.

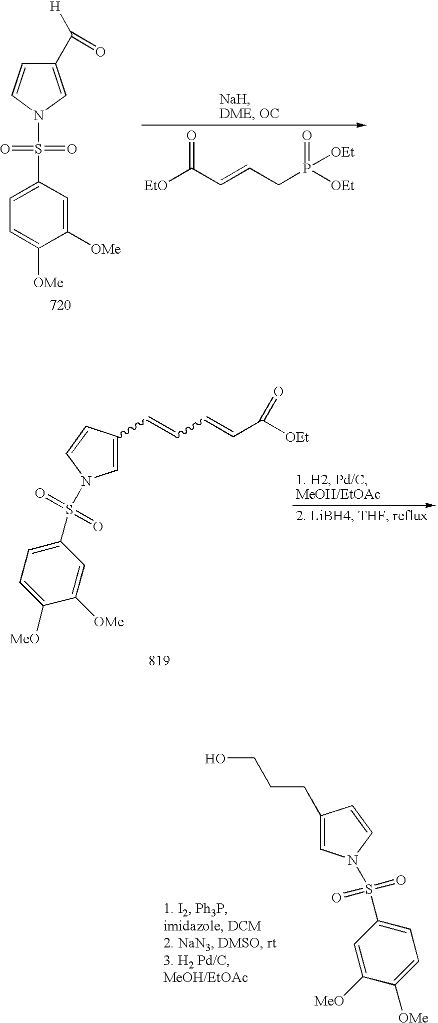

### EXAMPLE 551

### N-(5-(1-(3,4-dimethoxyphenylsulfonyl)-1H-pyrrol-3-yl)pentyl)-2-(4-fluorobenzyloxy)acetamide (822)

### Step 1: ethyl 5-(1-(3,4-dimethoxyphenylsulfonyl)-1H-pyrrol-3-yl)penta-2,4-dienoate (819)

Sodium hydride (34 mg, 60% in oil, 0.85 mmol) was added to a mixture of aldehyde 720 (210 mg, 0.71 mmol), (E)-ethyl 4-(diethoxyphosphoryl)but-2-enoate (214 mg, 0.85 mmol, 1.2 eq) in DME (4 ml) in ice-bath under N₂. The reaction was allowed to warm-up slowly to room temperature and was stirred at room temperature for 16 h. Sat'd NaHCO₃ and EtOAc were added and the organic layer was separated, dried (MgSO₄), filtered and concentrated leaving brown oil. The crude material was purified by silica gel column chromatography with gradient of EtOAc (25-30%) in hexanes to give 819 as white solid (43% yield, the trans isomer major). LRMS (ESI): (calc.) 391.4, (found) 392.0 (MH)⁺

### Step 2: 5-(1-(3,4-dimethoxyphenylsulfonyl)-1H-pyrrol-3-yl)pentan-1-ol (820)

The unsaturated ester 819 (115 mg, 0.29 mmol) and 10% Pd/C (28 mg) in EtOAc (7 ml) and MeOH (5 ml) was hydrogenated at 1 atmosphere of H₂ gas for 6 h. Ethyl 5-(1-(3,4-dimethoxyphenylsulfonyl)-1H-pyrrol-3-yl)pentanoate, was obtained as clear oil (97% yield).

LRMS (ESI): (calc.) 395.5; (found) 396.1 (MH)⁺

LiAlH₄ (11.4 mg, 0.3 mmol) was added to a solution of the ester above (113 mg, 0.286 mmol) in THF (3 ml) and the reaction was refluxed for 1 h, then it was stirred at room temperature for 16 h. The progress of the reaction was slow, so LiBH₄ (2M solution, 0.15 ml) was added and the mix was refluxed for 2 h, then it was cooled, quenched with H₂O and extracted with EtOAc, and the organic layer separated and washed with brine, dried (MgSO₄), filtered and concentrated giving 820 as clear oil in almost quantitative yield. LRMS (ESI): (calc.) 353.4; (found) 354.0 (MH)⁺

### Step 3: 5-(1-(3,4-dimethoxyphenylsulfonyl)-1H-pyrrol-3-yl)pentan-1-amine (821)

Iodine (223.4 mg, 0.88 mmol, 1.1 eq.) was added to a mixture of Ph₃P (210 mg, 0.8 mmol, 1 eq) and imidazole (54.5 mg, 0.8 mmol) in DCM (4 ml) at room temperature under N₂. The mixture became yellow. Alcohol 820 (283 mg, 0.8 mmol) in DCM (1 ml) was added and the mixture was stirred at room temperature for 16 h. The mixture was taken to dryness and the residue was purified by silica gel column chromatography with gradient of EtOAc (20-50%) in hexanes to give 1-(3,4-dimethoxyphenylsulfonyl)-3-(5-iodopentyl)-1H-pyrrole (62% yield) as a light pink oil which became dark upon standing. The material was used as is for next step. LRMS (ESI): (calc.) 463.3; (found) 464.0 (MH)⁺

Sodium azide (65.7 mg, 1.01 mmol) was added to a solution of the iodo compound from above (234 mg, 0.51 mmol) in DMSO (2 ml) and the reaction mixture was stirred at room temperature for 16 h. EtOAc was added and the organic phase was washed with H₂O (×3), brine, dried (MgSO₄), filtered and concentrated to give 3-(5-azidopentyl)-1-(3,4-dimethoxyphenylsulfonyl)-1H-pyrrole. LRMS (ESI): (calc.) 378.4; (found) 379.1 (MH)⁺

The crude azide from above and 10% Pd/C (35 mg) in MeOH (5 ml) and EtOAc (5 ml) was hydrogenated with H₂ (1 atmosphere). After 6 h, the catalyst was filtered (Celite) and the material taken to dryness to give 821 (145 mg). LRMS (ESI): (calc.) 352.4; (found) 353.1 (MH)⁺

Step 4: N-(5-(1-(3,4-dimethoxyphenylsulfonyl)-1H-pyrrol-3-yl)pentyl)-2-(4-fluorobenzyloxy)acetamide (822)

Acid chloride 289 (202 mg, 1 mmol, 1.3 eq.) was added to amine 821 (145 mg, 0.41 mmol) and TEA (202.4 mg/279 uL, 2 mmol) in THF (3 ml) as described for compound 723, example 507, scheme 302, step 4. The crude material was purified by silica gel column chromatography with gradient of EtOAc (40-100%) in hexanes to give 822 (83% yield). (CDCl₃) δ (ppm) ¹H: 7.46 (dxd, J=2.2, 8.6 Hz, 1H), 7.3 (m, 3H), 7.06 (m, 3H), 6.9 (d, J=8.6 Hz, 1H), 6.88 (s, 1H), 6.54 (bs, 1H), 6.13 (s, 1H), 4.53 (s, 2H), 3.97 (s, 2H), 3.92 (s, 3H), 3.91 (s, 3H), 3.26 (quartet, J=6.8 Hz, 2H), 2.38 (t, J=7.5 Hz, 2H), 1.52 (m, 4H), 1.32 (m, 2H). LRMS (ESI): (calc.) 518.6; (found) 519.2 (MH)⁺

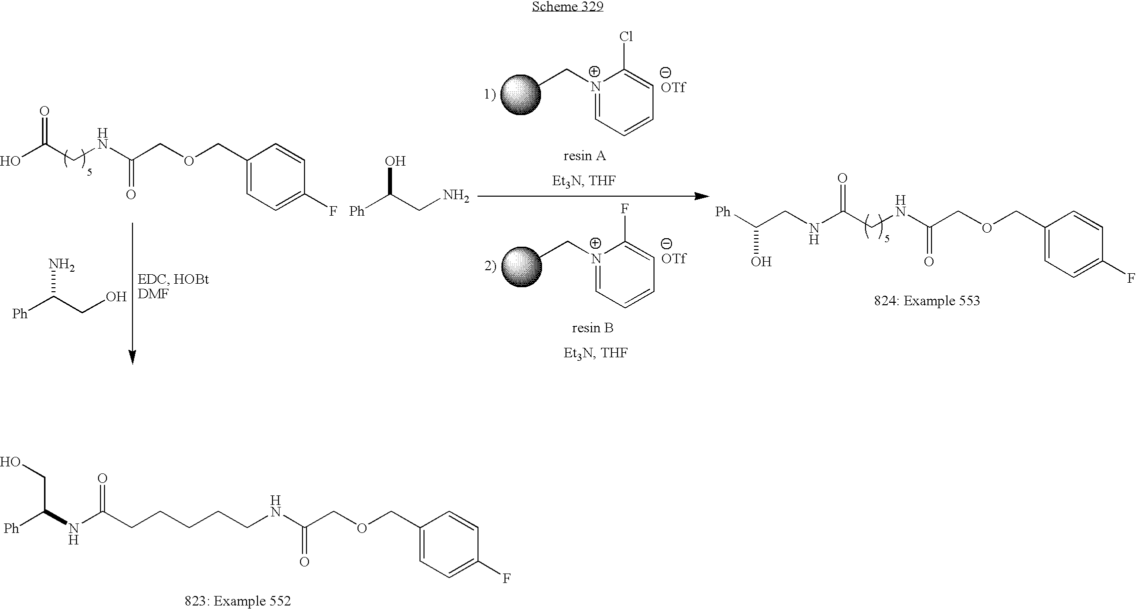

### EXAMPLE 552

(S)-6-(2-(4-fluorobenzyloxy)acetamido)-N-(2-hydroxy-1-phenylethyl)hexanamide (823)

To a solution of 6-(2-(4-fluorobenzyloxy)acetamido)hexanoic acid (301 mg, 1.01 mmol), (S)-2-phenylglycinol (153 mg, 1.11 mmol), and HOBt (309 mg, 2.02 mmol) in DMF (3 mL) was added EDC (387 mg, 2.02 mmol). The reaction was stirred for 1.75 hours at room temperature and then concentrated. The residue was partitioned between ethyl acetate and 1 N HCl(aq), and the organic phase was washed with NaHCO₃ (sat'd), dried over (Na₂SO₄) filtered and concentrated. The residue was purified by silica gel flash chromatography using a 25M Biotage column with a gradient of methanol (0-15%) in DCM to obtain 823 as a white solid (187 mg, 44%).

(DMSO-d₆) δ (ppm) ¹H: 8.14 (d, J=8.0 Hz, 1H), 7.77 (t, J=5.6 Hz, 1H), 7.42-7.39 (m, 2H), 7.30-7.25 (m, 4H), 7.20-7.15 (m, 3H), 4.83-4.77 (m, 2H), 4.48 (s, 2H), 3.84 (s, 2H), 3.51-3.48 (m, 2H), 3.04 (q, J=6.4 Hz, 2H), 2.11 (t, J=7.6 Hz, 2H), 1.46 (quint. J=7.6 Hz, 2H), 1.37 (quint J=7.2 Hz, 2H), 1.22-1.16 (m, 2H). LRMS (ESI): (calc) 416.2; (found) 417.1 (MH)⁺, 439.1 (MNa)⁺.

### EXAMPLE 553

### (R)-6-(2-(4-fluorobenzyloxy)acetamido)-N-(2-hydroxy-2-phenylethyl)hexanamide (824)

To a solution of 6-(2-(4-fluorobenzyloxy)acetamido)hexanoic acid (63 mg, 0.212 mmol), (R)-2-amino-1-phenylethanol (35 mg, 0.254 mmol) in THF (4 mL) and DMF (0.5 mL) was added triethylamine (90uL, 0.636 mmol) then Resin A (0.424 mmol) The suspension was stirred for 2 hours before the addition of Resin B (0.424 mmol) and 1.0 mL of triethylamine according to the procedure of S. Crosignani and D. Swinnen (J. Comb. Chem. 2005, 7(5) 688-696). After another 3 days of stirring the suspension was filtered through an amino-functionalized SPE column (Isolute NH₂ SPE, 2 g). The filterate was concentrated and then purified by silica gel flash chromatography using a 12M Biotage column and a gradient of methanol (0-20%) in DCM and then triturated with ethyl acetate and hexanes to obtain 824 as a white solid (17 mg). (DMSO-d₆) δ (ppm) ¹H, 7.83 (t, J=5.6 Hz, 1H), 7.77 (t, J=5.6 Hz, 1H), 7.42-7.39 (m, 2H), 7.30-7.28 (m, 4H), 7.26-7.15 (m, 3H), 5.43 (d, J=4.4 Hz, 1H), 4.58-4.54 (m, 1H), 4.49 (s, 2H), 3.84 (s, 2H), 3.28-3.22 (m, 1H), 3.10-3.02 (m, 3H), 2.03 (t, J=7.2 Hz, 2H), 1.47-1.33 (m, 4H), 1.19-1.13 (m, 2H). LRMS (ESI): (calc) 416.2; (found) 417.1 (MH)⁺, 439.1 (MNa)⁺.

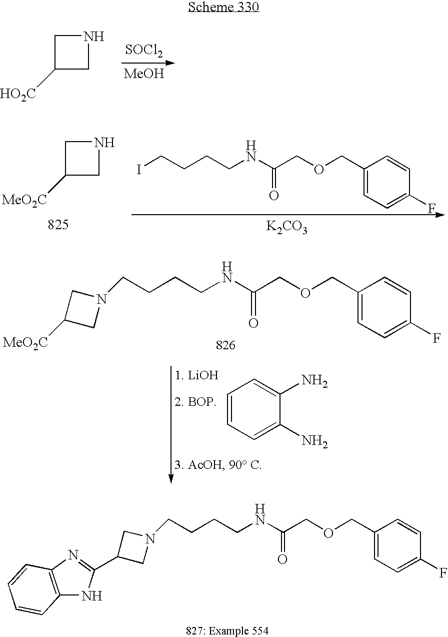

### EXAMPLE 554

### N-(4-(3-(1H-benzo[d]imidazol-2-yl)azetidin-1-yl)butyl)-2-(4-fluorobenzyloxy)acetamide (827)

### Step 1: Methyl azetidine-3-carboxylate (825)

To a stirred solution of azetidine-3-carboxylic acid (325 mg, 3.21 mmol) in MeOH (15 mL) was carefully added SOCl₂ (0.47 mL, 6.43 mmol). The solution was stirred at room temperature for 1 h and concentrated to yield 825 (365 mg, 99%) as a clear oil. LRMS (ESI): (calc) 115.1; (found) 116.2 (MH)⁺.

### Step 2: Methyl 1-(4-(2-(4-fluorobenzyloxy)acetamido)butyl)azetidine-3-carboxylate (826)

The ester 825 (365 mg, 3.18 mmol) was partially dissolved in MeCN (15 mL) and K₂CO₃ (1.33 g, 9.64 mmol) was added followed by 2-(4-fluorobenzyloxy)-N-(4-iodobutyl)acetamide (1.17 g, 3.21 mmol). The suspension was stirred at room temperature for 2 h, H₂O was added and the aqueous phase was extracted with EtOAc and the EtOAc extracts were filtered and evaporated to afford 826 (825 mg, 73%). LRMS (ESI): (calc) 352.4; (found) 353.2 (MH)⁺.

### Step 3: N-(4-(3-(1H-benzo[d]imidazol-2-yl)azetidin-1-yl)butyl)-2-(4-fluorobenzyloxy)acetamide (827)

To a stirred solution of the ester 826 (88 mg, 0.25 mmol) in a 1:1 mixture of H₂O/THF (1.3 mL) was added LiOH (12 mg, 0.50 mmol). After stirring for 1 h, the mixture was filtered through a Celite-SiO₂ pad and washed with 30% MeOH in CH₂Cl₂. The filtrate was evaporated to dryness. To a stirred solution of the crude acid in DMF (2.5 mL) were added BOP (121 mg, 0.27 mmol), 1,2-phenylenediamine (27 mg, 0.25 mmol), and Et₃N (0.08 mL, 0.55 mmol). The solution was stirred overnight at room temperature. H₂O was added and the aqueous phase was extracted with EtOAc. The organic extracts were combined, dried and evaporated. The residue obtained was dissolved in AcOH (2.5 mL) and heated at 90° C. for 10 minutes. The solution was evaporated to dryness and the residue was purified by Prep-HPLC to afford 827 (11 mg, 10%) as the formate salt. (CD₃CN) δ (ppm) ¹H: 7.52 (dd, J=3.2, 6.0 Hz, 2H), 7.39 (dd, J=5.6, 8.8 Hz, 2H), 7.22 (dd, J=3.2, 6.0 Hz, 2H), 7.06 (t, J=8.8 Hz, 2H), 4.56 (s, 2H), 4.05-3.99 (m,1H), 3.95 (t, J=7.6 Hz, 2H), 3.93 (s, 2H), 3.68 (t, J=7.2 Hz, 2H), 3.26 (t, J=6.8 Hz, 2H), 2.75 (t, J=7.6 Hz, 2H), 1.57 (qt, J=7.2 Hz, 2H), 1.46 (qt, J=8.4 Hz, 2H). LRMS (ESI): (calc) 410.5; (found) 411.2 (MH)⁺.

Compositions

In a second aspect, the invention provides compositions comprising an inhibitor of histone deacetylase according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain preferred embodiments, compounds of the invention are administered intravenously in a hospital setting. In certain other preferred embodiments, administration may preferably be by the oral route. The compositions may be in the form of liquid solutions or suspensions; for oral administration, formulations may be in the form of tablets or capsules; and for intranasal formulations, in the form of powders, nasal dropsa or aerosols. The compositions of the invention may be administered systemically or locally.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salts” is intended to mean salts that retain the desired biological activity of the above-identified compounds and exhibit minimal or no undesired toxicological effects. Examples of such salts include, but are not limited to acid addition salts formed with inorganic acids (for Example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compound is included in the pharmaceutically acceptable carrier or diluent in an amount sufficient to deliver an inhibition effective amount without causing serious toxic effects. A preferred dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

In certain preferred embodiments of the second aspect of the invention, the composition further comprises an antisense oligonucleotide that inhibits the expression of a histone deacetylase gene. The combined use of a nucleic acid level inhibitor (e.g., antisense oligonucleotide) and a protein level inhibitor (i.e., inhibitor of histone deacetylase enzyme activity) results in an improved inhibitory effect, thereby reducing the amounts of the inhibitors required to obtain a given inhibitory effect as compared to the amounts necessary when either is used individually. The antisense oligonucleotides according to this aspect of the invention are complementary to regions of RNA or double-stranded DNA that encode HDAC-1, HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7 (see e.g., GenBank Accession Number U50079 for HDAC-1, GenBank Accession Number U31814 for HDAC-2, and GenBank Accession Number U75697 for HDAC-3).

Inhibition of Histone Deacetylase

In a third aspect, the present invention provides a method of inhibiting histone deacetylase, comprising contacting the histone deacetylase with an inhibition effective amount of an inhibitor of histone deacetylase of the present invention.

In another embodiment of the third aspect, the invention provides a method of inhibiting histone deacetylase in a cell, comprising contacting the cell in which inhibition of histone deacetylase is desired with an inhibition effective amount of an inhibitor of histone deacetylase, or composition thereof, according to the present invention.

Because compounds of the invention inhibit histone deacetylase, they are useful research tools for in vitro study histone deacetylases and their role in biological-processes.

Measurement of the enzymatic activity of a histone deacetylase can be achieved using known methodologies. For Example, Yoshida et al., J. Biol. Chem., 265: 17174-17179 (1990), describes the assessment of histone deacetylase enzymatic activity by the detection of acetylated histones in trichostatin A treated cells. Taunton et al., Science, 272: 408-411 (1996), similarly describes methods to measure histone deacetylase enzymatic activity using endogenous and recombinant HDAC-1.

In some preferred embodiments, the histone deacetylase inhibitor interacts with and reduces the activity of all histone deacetylases in a cell. In some other preferred embodiments according to this aspect of the invention, the histone deacetylase inhibitor interacts with and reduces the activity of fewer than all histone deacetylases in the cell. In certain preferred embodiments, the inhibitor interacts with and reduces the activity of one histone deacetylase (e.g., HDAC-1), but does not interact with or reduce the activities of other histone deacetylases (e.g., HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7).

The term “inhibition effective amount” is meant to denote a dosage sufficient to cause inhibition of histone deacetylase activity in a cell, which cell can be in a multicellular organism. The multicellular organism can be a plant or an animal, preferably a mammal. If in a multicellular organism, the method according to this aspect of the invention comprises administering to the organism a compound or composition according to the present invention. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

In certain preferred embodiments of the third aspects of the invention, the method further comprises contacting a histone deacetylase enzyme or a cell expressing histone deacetylase activity with an antisense oligonucleotide that inhibits the expression of a histone deacetylase gene. The combined use of a nucleic acid level inhibitor (e.g., antisense oligonucleotide) and a protein level inhibitor (i.e., inhibitor of histone deacetylase enzyme activity) results in an improved inhibitory effect, thereby reducing the amounts of the inhibitors required to obtain a given inhibitory effect as compared to the amounts necessary when either is used individually. The antisense oligonucleotides according to this aspect of the invention are complementary to regions of RNA or double-stranded DNA that encode HDAC-1, HDAC-2, HDAC-3, HDAC4, HDAC-5, HDAC-6, HDAC-7, HDAC-8, SirT1, SirT2, SirT3, SirT4, SirT5, SirT6 and SirT7 (see e.g., GenBank Accession Number U50079 for HDAC-1, GenBank Accession Number U31814 for HDAC-2, and GenBank Accession Number U75697 for HDAC-3).

For purposes of the invention, the term “oligonucleotide” includes polymers of two or more deoxyribonucleosides, ribonucleosides, or 2′-substituted ribonucleoside residues, or any combination thereof. Preferably, such oligonucleotides have from about 6 to about 100 nucleoside residues, more preferably from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate and sulfone internucleoside linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines and adamantane.

For purposes of the invention the term “2′-substituted ribonucleoside” includes ribonucleosides in which the hydroxyl group at the 2′ position of the pentose moiety is substituted to produce a 2′-O-substituted ribonucleoside. Preferably, such substitution is with a lower alkyl group containing 1-6 saturated or unsaturated carbon atoms, or with an aryl or allyl group having 2-6 carbon atoms, wherein such alkyl, aryl or allyl group may be unsubstituted or may be substituted, e.g., with halo, hydroxy, trifluoromethyl, cyano, nitro, acyl, acyloxy, alkoxy, carboxyl, carbalkoxyl, or amino groups. The term “2′-substituted ribonucleoside” also includes ribonucleosides in which the 2′-hydroxyl group is replaced with an amino group or with a halo group, preferably fluoro.

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred example of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al. U.S. Pat. Nos. 5,635,377 and 5,366,878). Preferably, such chimeric oligonucleotides contain at least three consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred example of such a hybrid oligonucleotide comprises a ribonucleotide or 2′-substituted ribonucleotide region, preferably comprising from about 2 to about 12 2′-substituted nucleotides, and a deoxyribonucleotide region. Preferably, such a hybrid oligonucleotide contains at least three consecutive deoxyribonucleosides and also contains ribonucleosides, 2′-substituted ribonucleosides, preferably 2′-O-substituted ribonucleosides, or combinations thereof (see e.g., Metelev and Agrawal, U.S. Pat. No. 5,652,355).

The exact nucleotide sequence and chemical structure of an antisense oligonucleotide utilized in the invention can be varied, so long as the oligonucleotide retains its ability to inhibit expression of the gene of interest. This is readily determined by testing whether the particular antisense oligonucleotide is active. Useful assays for this purpose include quantitating the mRNA encoding a product of the gene, a Western blotting analysis assay for the product of the gene, an activity assay for an enzymatically active gene product, or a soft agar growth assay, or a reporter gene construct assay, or an in vivo tumor growth assay, all of which are described in detail in this specification or in Ramchandani et al. (1997) Proc. Natl. Acad. Sci. USA 94: 684-689.

Antisense oligonucleotides utilized in the invention may conveniently be synthesized on a suitable solid support using well known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T. (1993) Methods in Molec. Biol. 20: 465-496).

Particularly preferred oligonucleotides have nucleotide sequences of from about 13 to about 35 nucleotides which include the nucleotide sequences shown in Table 44. Yet additional particularly preferred oligonucleotides have nucleotide sequences of from about 15 to about 26 nucleotides which include the nucleotide sequences shown in Table 44.

TABLE 44

position

| | | |
|---|---|---|
| Accession | Nucleotide | within |

| | | | | | | |
|---|---|---|---|---|---|---|
| Oligo | Target | Number | Position | Sequence | Gene | Seq ID No. |

| | | | | | | |
|---|---|---|---|---|---|---|
| HDAC1 AS1 | Human HDAC1 | U50079 | 1585-1604 | 5′-GAAACGTGAGGGACTCAGCA-3′ | 3′-UTR | Seq ID No:1 |
| HDAC1 AS2 | Human HDAC1 | U50079 | 1565-1584 | 5′-GGAAGCCAGAGCTGGAGAGG-3 | 3′-UTR | Seq ID No:2 |
| HDAC1 MM | Human HDAC1 | U50079 | 1585-1604 | 5′-GTTAGGTGAGGCACTGAGGA-3′ | 3′-UTR | Seq ID No:3 |
| HDAC2 AS | Human HDAC2 | U31814 | 1643-1622 | 5′-GCTGAGCTGTTCTGATTTGG-3′ | 3′-UTR | Seq ID No:4 |
| HDAC2 MM | Human HDAC2 | U31814 | 1643-1622 | 5′-CGTGAGCACTTCTCATTTCC-3′ | 3′-UTR | Seq ID No:5 |
| HDAC3 AS | Human HDAC3 | AF039703 | 1276-1295 | 5′-CGCTTTCCTTGTCATTGACA-3′ | 3′-UTR | Seq ID No:6 |
| HDAC3 MM | Human HDAC3 | AF039703 | 1276-1295 | 5′-GCCTTTCCTACTCATTGTGT-3′ | 3′-UTR | Seq ID No:7 |
| HDAC4 AS1 | Human HDAC4 | AB006626 | 514-33 | 5′-GCTGCCTGCCGTGCCCACCC-3′ | 5′-UTR | Seq ID No:8 |
| HDAC4 MM1 | Human HDAC4 | AB006626 | 514-33 | 5′-CGTGCCTGCGCTGCCCACGG-3′ | 5′-UTR | Seq ID No:9 |
| HDAC4 AS2 | Human HDAC4 | AB006626 | 7710-29 | 5′-TACAGTCCATGCAACCTCCA-3′ | 3′-UTR | Seq ID No:10 |
| HDAC4 MM4 | Human HDAC4 | AB006626 | 7710-29 | 5′-ATCAGTCCAACCAACCTCGT-3′ | 3′-UTR | Seq ID No:11 |
| HDAC5 AS | Human HDAC5 | AF039691 | 2663-2682 | 5′-CTTCGGTCTCACCTGCTTGG-3′ | 3′-UTR | Seq ID No:12 |
| HDAC6 AS | Human HDAC6 | AJ011972 | 3791-3810 | 5′-CAGGCTGGAATGAGCTACAG-3′ | 3′-UTR | Seq ID No:13 |
| HDAC6 MM | Human HDAC6 | AJ011972 | 3791-3810 | 5′-GACGCTGCAATCAGGTAGAC-3′ | 3′-UTR | Seq ID No:14 |
| HDAC7 AS | Human HDAC7 | AF239243 | 2896-2915 | 5′-CTTCAGCCAGGATGCCCACA-3′ | 3′-UTR | Seq ID No:15 |
| HDAC8 AS1 | Human HDAC8 | AF230097 | 51-70 | 5′-CTCCGGCTCCTCCATCTTCC-3′ | 5′-UTR | Seq ID No:16 |
| HDAC8 AS2 | Human HDAC8 | AF230097 | 1328-1347 | 5′-AGCCAGCTGCCACTTGATGC-3′ | 3′-UTR | Seq ID No:17 |

In certain preferred embodiments of the invention, the antisense oligonucleotide and the HDAC inhbitor of the present invention are administered separately to a mammal, preferably a human. For example, the antisense oligonucleotide may be administered to the mammal prior to administration to the mammal of the HDAC inhibitor of the present invention. The mammal may receive one or more dosages of antisense oligonucleotide prior to receiving one or more dosages of the HDAC inhibitor of the present invention.

In another embodiment, the HDAC inhibitor of the present invention may be administered to the mammal prior to administration of the antisense oligonucleotide. The mammal may receive one or more dosages of the HDAC inhibitor of the present invention prior to receiving one or more dosages of antisense oligonucleotide.

In certain preferred embodiments of the present invention, the HDAC inhibitor of the present invention may be administered together with other HDAC inhibitors known in the art or which will be discovered. Administration of such HDAC inhibitors may be done sequentially or concurrently. In certain preferred embodiments of the present invention the compositions comprise an HDAC inhibitor of the present invention and/or an antisense oligonucleotide and/or another HDAC inhibitor known in the art or which will be discovered. The active ingredients of such compositions may act synergistically to produce a therapeutic effect.

In certain embodiments, the known HDAC inhibitor is selected from the group consisting of trichostatin A, depudecin, trapoxin, suberoylanilide hydroxamic acid, FR901228, MS-27-275, CI-994 sodim butyrate, and those compounds found in WO 2003/024448, WO 2001/038322, U.S. Pat. No. 6,541,661, WO 01/70675, and PCT/US04/31591.

The following Examples are intended to further illustrate certain preferred embodiments of the invention, and are not intended to limit the scope of the invention.

### ASSAY EXAMPLES

### Assay Example 1

Inhibition of Histone Deacetylase Enzymatic Activity

The following protocol is used to assay the compounds of the invention. In the assay, the buffer used is 25 mM HEPES, pH 8.0, 137 mM NaCl, 2.7 mM KCl, 1 mM MgCl₂ and the subtrate is Boc-Lys(Ac)-AMC in a 50 mM stock solution in DMSO. The enzyme stock solution is 4.08 μg/mL in buffer.

The compounds are pre-incubated (2 μl in DMSO diluted to 13 μl in buffer for transfer to assay plate) with enzyme (20 μl of 4.08 μg/ml) for 10 minutes at room temperature (35 μl pre-incubation volume). The mixture is pre-incubated for 5 minutes at room temperature. The reaction is started by bringing the temperature to 37° C. and adding 16 μl substrate. Total reaction volume is 50 μl. The reaction is stopped after 20 minutes by addition of 50 μl developer, prepared as directed by Biomol (Fluor-de-Lys developer, Cat. # KI-105). A plate is incubated in the dark for 10 minutes at room temperature before reading (λ_{Ex}=360 nm, λ_{Em}=470 nm, Cutoff filter at 435 nm).

All compounds exemplified have an IC₅₀ value less than or equal to 10 μM against one or more of HDAC-1, HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7. Tables 45-50, for example, show selected examples.

TABLE 45

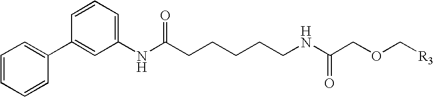

| Cpd | R₃ |
|---|---|
| 61 | Ph |
| 7b | 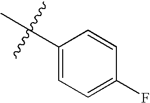 |
| 47 | 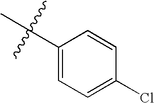 |
| 18 | 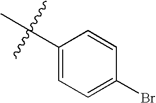 |
| 13 | 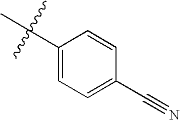 |
| 14 | 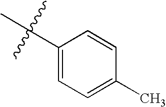 |
| 50 | 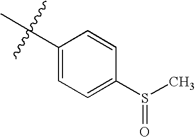 |
| 15 | 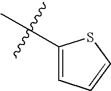 |
| 16 | 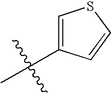 |
| 17 | 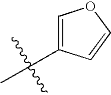 |
| 45 | 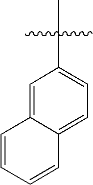 |
| 19 | 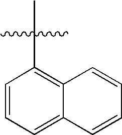 |

TABLE 46

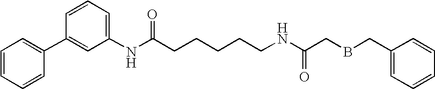

| Cpd | B |
|---|---|
| 51 | —S— |
| 52 | —S(O)— |

TABLE 47

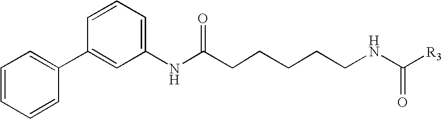

| Cpd | R₃ |
|---|---|
| 98 | 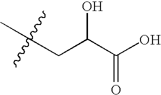 |
| 33 | 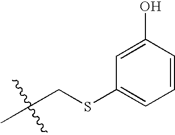 |
| 29 | 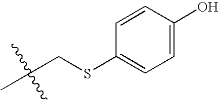 |
| 34 | 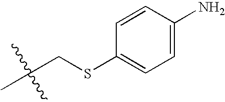 |
| 35 | 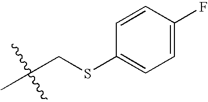 |
| 36 | 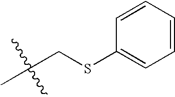 |
| 37 | 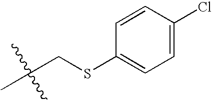 |
| 38 | 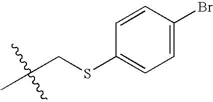 |
| 39 | 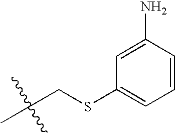 |
| 40 | 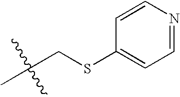 |
| 41 | 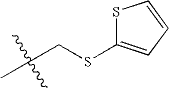 |
| 83 | 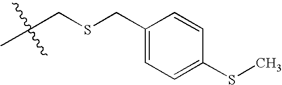 |
| 77 | 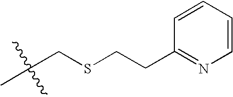 |
| 84 | 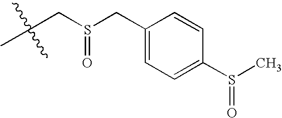 |
| 56 | 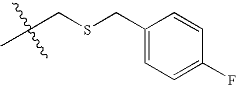 |
| 57 | 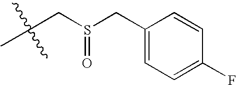 |
| 24 | 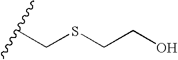 |
| 23 | 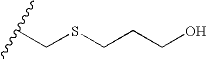 |
| 100 | 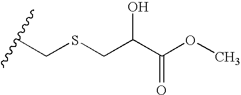 |
| 5 | 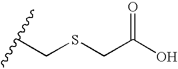 |
| 21 | 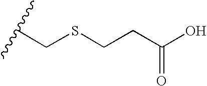 |
| 82 | 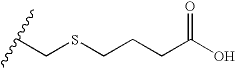 |
| 26 | 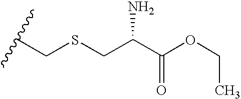 |
| 27 | 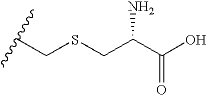 |
| 4 | 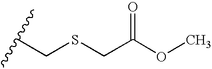 |
| 81 | 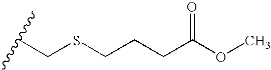 |
| 30 | 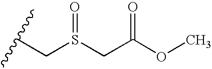 |
| 22 | 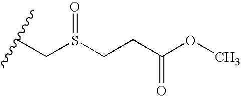 |
| 59 | 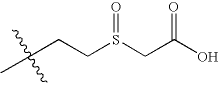 |
| 25 | 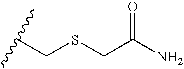 |
| 31 | 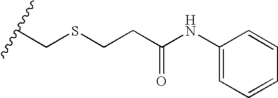 |
| 20 | 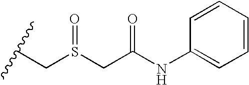 |
| 32 | 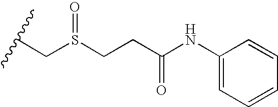 |
| 28 | 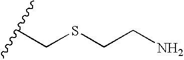 |
| 78 | 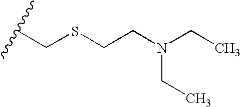 |

TABLE 48

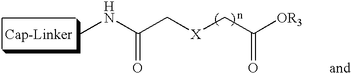

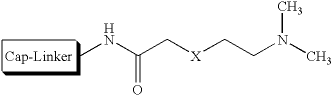

| Cpd | Structure |
|---|---|
| 113 | 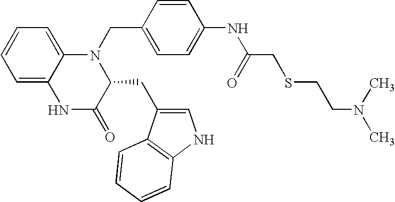 |
| 76 | 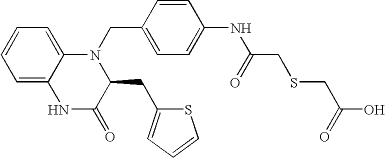 |
| 91 | 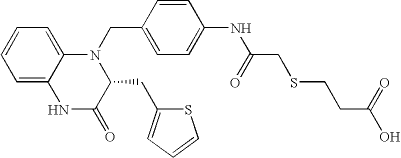 |
| 90 | 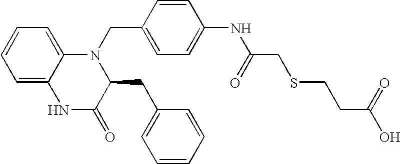 |
| 108 | 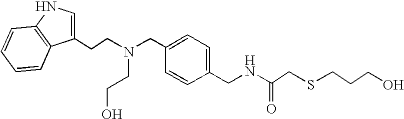 |

TABLE 49

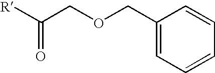

| Cpd | R′ |
|---|---|
| 64 | 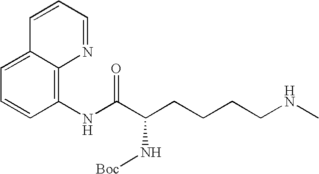 |
| 66 | 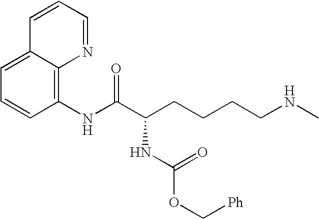 |

TABLE 50

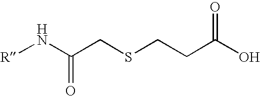

| Cpd | Structure |
|---|---|
| 94 | 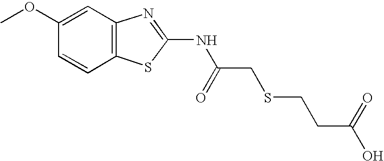 |
| 118 | 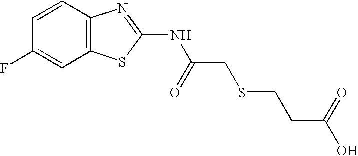 |
| 119 | 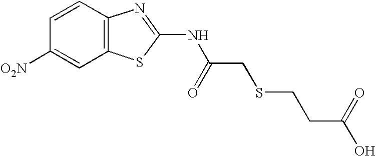 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of the formula 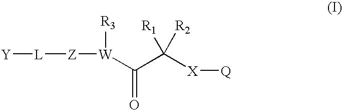
or pharmaceutically acceptable salts thereof, wherein
W is nitrogen or oxygen, wherein when W is oxygen, R₃ is absent;
X is a covalent bond, —S—, —SO—, —SO₂—, —O—, —NR₃—, —CH₂—, —N(OH), optionally substituted —C₁-C₆ alkyl, or a structure of the formula 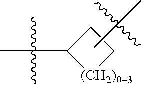
R₁ and R₂ are independently selected from the group consisting of —H, C₁-C₆ alkyl, halo; —N(H)—C(O)—O—C₁-C₆ alkyl, —N(H)—C(O)—O-benzyl, C₃-C₆ cycloalkyl, aryl, aryl-C₁-C₆ alkyl-, and heteroaryl-C₁-C₆ alkyl, wherein the alkyl, benzyl, cycloalkyl, aryl and heteroaryl moieties of said R₁ and R₂ are optionally substituted; or
R₁ and R₂ together with the carbon atom to which they are attached form a 3 to 9-membered heterocyclyl-aryl, C₃-C₆-cycloalkyl or 3 to 9-membered heterocyclyl group, wherein each of the cycloalkyl, heterocyclyl, and heterocyclyl-aryl is optionally substituted with one or more groups selected from oxo, —OH, —CN, C₁-C₆ alkyl, C₁-C₆ alkoxy, —NO₂, —N(R₃)(R₃ₐ), halo, —SH, mono- to per-halogenated C₁-C₆ alkyl; or
when X-Q is absent, R₁ and R₂ together with the atom to which they are attached form an aryl, heterocyclyl, cycloalkyl or heteroaryl group, wherein said aryl, heterocyclyl, cycloalkyl and heteroaryl are optionally substituted, and wherein R³ is optionally connected to the aryl, heterocyclyl, cycloalkyl or heteroaryl by a covalent bond;
X-Q, R₃ and R₃ₐ are independently selected from the group consisting of —H, —OH, —C(O)H, heterocyclyl, C₁-C₆alkyl, C₂-C₆alkenyl, C₂-C₃alkynyl, C₂-C₄ alkyl-OR₃, C₂-C₆ hydroxyalkyl, heteroaryl, C₁-C₆heteroalkyl-aryl, C₀-C₆alkylheteroaryl, C₀-C₆heteroalkylheteroaryl, C₁-C₃alkyl-C(O)NR₃-heteroaryl, C₁-C₃alkyl-C(O)NR₃-aryl, C₁-C₄alkyl-C(O)OR₃, —C₁-C₆ hydroxyalkyl-C(O)—OH, —C(O)—NH-aryl, —C(O)CF₃, —C(O)—NH₂, —CH(NH₂)—C(O)—OH, —NH₂, —CH(NH₂)—C(O)—O—C₁-C₆ alkyl, —C(O)—OH, —C(O)—O—C₁-C₆ alkyl, C₁-C₆ heteroalkyl-C₁-C₆ alkyl-, C₃-C₆ cycloalkyl, heterocyclyl-C₁-C₆ alkyl-, —C₁-C₆ alkylaryl, aryl, —C₀-C₆ alkyl-S(O)—C₁-C₆ alkylaryl, —C₀-C₆ alkyl-O—C₀-C₆ alkylaryl, —C₀-C₆ alkyl-S—C₀-C₆ alkylaryl, —C₀-C₆ alkyl-S—C₀-C₆ alkylheteroaryl, —C₀-C₆ alkyl-S—C₁-C₆ alkyl-C(O)—OH, —C₀-C₆ alkyl-S—C₁-C₆ hydroxyalkyl-C(O)—O—C₁-C₆ alkyl, —C₀-C₆ alkyl-S—C₁-C₆ alkyl-CH(NH₂)—C(O)—OR₄, —C₀-C₆ alkyl-S—C₁-C₆ alkyl-OH, —C₀-C₆ alkyl-S—C₁-C₆ alkyl-C(O)—O—C₁-C₆ alkyl, —C₀-C₆ alkyl-S(O)—C₁-C₆ alkyl-C(O)—OR₄, —C₀-C₆ alkyl-S(O)—C₁-C₆ alkyl-C(O)—N(R₄)-aryl, —C₀-C₆ alkyl-S—C₁-C₆ alkyl-C(O)—N(R₄)(R₄ₐ), —C₀-C₆ alkyl-S—C₁-C₆ alkyl-N(R₄)(R₄ₐ), —C₁-C₆ alkylheteroaryl and heteroaryl, wherein each alkyl, alkenyl, alkynyl, heteroalkyl, heteroalkyl, cycloalkyl, heterocyclyl, aryl and heteroaryl moiety of the aforementioned X-Q, R³ and R^{3a} is optionally substituted;
Q is selected from the group consisting of —H, —OH, —N(R₃)(R₃ₐ), halo, —SH, —C(O)OR₃, —C(O)R₃, —C₀-C₃-alkyl-diphenyl-R₄, —C₀-C₃alky-aryl, —C₀-C₃alkyl-heteroaryl, —C₁-C₆ alkyl-N(R₃)—C(O)-C₁-C₆ alkyl-B—(CH₂)ₙ—R₃, —C₁-C₆-alkyl-N(R₃)—C(O)-C₁-C₆ alkyl-O-C₁-C₆ alkyl-R₃, —C₁-C₆-alkyl-C(O)—OR₃, —C₁-C₆-alkyl-N(R₃)(R₃ₐ), —C₁-C₆-alkyl-CN, —C₁-C₆ alkyl-C(O)—N(R₃)—N(R₃)-aryl, —C₁-C₆ alkyl-N(R₃)—C₁-C₆ heteroalkyl, —C₁-C₆ alkyl-N(R₃)—C₃-C₆ cycloalkyl, —C₁-C₆ alkyl-N(R₃)-heterocyclyl, —C₁-C₆ alkyl-N(R₃)-aryl, —C₁-C₆ alkyl-N(R₃)—C₁-C₆-alkyl-aryl, —C₁-C₆ alkyl-N(R₃)-heteroaryl, —C₁-C₆ alkyl-N(R₃)—C₃-C₆ cycloalkyl, C₁-C₆ alkyl substituted with —OH, —C₁-C₆ alkyl-O—C₁-C₆ alkyl, —C₁-C₆ alkyl-O—C₃-C₆ cycloalkyl, —C₁-C₆-alkyl-O-C₁-C₆ alkyl-(C₁-C₆ heteroalkyl), —C₁-C₆-alkyl-O—C₁-C₆ alkyl-heterocyclyl, —C₁-C₆ alkyl-O-aryl, —C₁-C₆-alkyl-O—C₁-C₆ alkylaryl, —C₁-C₆ alkyl-O-heteroaryl, —C₁-C₆ alkylhydroxamate, C₁-C₆ alkyl, —C₁-C₆ alkyl-(C₁-C₆ heteroalkyl), C₃-C₆ cycloalkyl, —C₁-C₆ alkylheterocyclyl, —C₁-C₆ alkyl-C₂-C₆ alkenyl, —C₁-C₆ alkyl-C₂-C₆ alkynyl, aryl, —C₁-C₆ alkylaryl, heteroaryl, C₁-C₆ alkylheteroaryl, C₁-C₃ alkyl-CN, —C₁-C₆ alkyl-CH(OR₃)—C(O)—OR₃, —C₁-C₆ alkyl-CH(N(R₃)(R₃ₐ))—C(O)—OR₃, —C₁-C₆ alkyl-C(O)—N(R₃)(R₃ₐ), —C₁-C₆ alkyl-N(R₃)—C(O)—N(R₃)(R₃ₐ), —C₁-C₆ alkyl-N(R₃)—C(O)—OR₃, —C₁-C₆ alkyl-N(R₃)—C(O)—R₃, —C₁-C₆ alkyl-N(R₃)—S(O)₂—R₃, —C₁-C₆ alkyl-S(O)₂—N(R₃)(R₃ₐ), —C₁-C₆ alkyl-CH(N(R₃)(R₃ₐ))-C₁-C₆ alkyl-OR₃, or —C₁-C₆ alkyl-O—C(O)—N(R₃)(R₃ₐ), —C₁-C₆ alkyl-(C═NR₃)—N(R₃)(R₃ₐ), —C₁-C₆ alkyl-X—C₁-C₆ alkyl-C(O)OR₃, —C₁-C₆ alkyl-X—C₁-C₆ alkyl-OR₃, and —C₁-C₆ alkyl-X—C₁-C₆ alkyl-N(R₃)(R₃ₐ), C₁-C₄ alkyl-aryl- wherein the C₁-C₄ alkyl is optionally substituted with —C₁-C₄ alkylOR₃, C₁-C₄ alkylNR₃, R₃ₐ, C₀-C₄ alkylC(O)N(R₃)(R₃ₐ) or C₀-C₄ alkylC(O)OR₃, C₁-C₄ alkyl-heteroaryl- wherein the C₁-C₄ alkyl is optionally substituted with —C₁-C₄ alkylOR₃, C₁-C₄ alkylN(R₃)(R₃ₐ), C₀-C₄ alkylC(O)N(R₃)(R₃ₐ) or C₀-C₄ alkylC(O)OR₃, C₂-C₄ alkenyl, C₂-C₄ alkynyl, C₀-C₆ alkylC(O)NR₃—N(R₃)aryl and C₀-C₆ alkylC(O)NR₃—NR₃heteroaryl, wherein each alkyl, alkenyl, alkynyl, heteroalkyl, heteroalkyl, cycloalkyl, heterocyclyl, aryl and heteroaryl moiety of the aforementioned Q is optionally substituted;
B is selected from the group consisting of —O—, —S(O)—, —S— and —S(O)₂—,
n is 0 or an interger from 1 to 3;
R₄ and R₄ₐ are independently selected from the group consisting of —H, C₁-C₆ alkyl, C₂-C₆alkenyl, C₂-C₆alkynyl, C₁-C₆ alkyl-R₃, —C₀-C₆ alkyl-OR₃, —C₀-C₆ alkyl-C(O)—OR₃, —C₀-C₆ alkyl-C(O)NR₃, R₃ₐ, —CH═CH—C(O)—OR₃, —CH═CH—C(O)—N(R₃)(R₃ₐ), —N(R₃)—C(O)—CF₃, —N(R₃)—C₂-C₆ alkyl-N(R₃)(R₃ₐ), —C₀-C₆ alkyl-N(R₃)(R₃ₐ), —N(R₃)—C(O)—C₁-C₆ alkyl-R₃, —N(R₃)—S(O)₂—C₁-C₆ alkyl-R₃, —S(O)₂—N(R₃)R₃ₐ, —O—C₂-C₆ alkyl-N(R₃)(R₃ₐ), —S—R₃, —S(O)-C₁-C₆ alkyl-R₃, —S(O)₂—C₁-C₆ alkyl-R₃, C₃-C₆ cycloalkyl, heterocyclyl, C₄-C₇heterocyclyl-R₃, —O—C₂-C₄alkyl-heterocyclyl, —O-heterocyclyl-C(O)—OR₃, —NR₃—C₂-C₄alkyl-heterocyclyl, halo, —CF₃, —SO₃H, —CN, —C₁-C₆ alkylaryl, aryl, heteroaryl, —C₁-C₆ alkylheteroaryl, wherein each alkyl, alkenyl, alkynyl, cycloalkyl, heterocyclyl, aryl and heteroaryl moeity of the aformentioned R₄ and R₄ₐ are optionally substituted;
Z is selected from the group consisting of C₁-C₈ alkyl, C₁-C₈ alkenyl, C₁-C₈ alkynyl, C₁-C₈ heteroalkyl, —C₀-C₃alkyl-alkenyl-C₀-C₃-alkyl, —C₀-C₃alkyl-alkynyl-C₀-C₃-alkyl, —C₀-C₃alkyl-heteroalkyl-C₀-C₃-alkyl, aryl, —C₁-C₆ alkylaryl-, —C₀-C₆ alkylaryl-C₀-C₆-alkyl-, —C₀-C₆ alkylaryl-C₂-C₆-heteroalkyl-, —C₂-C₆ heteroalkylaryl-C₀-C₆-alkyl-, —C₄-C₆ heterocyclylaryl-C₀-C₆-alkyl-, —C₀-C₆ alkylaryl-C₄-C₆-heterocyclyl-, —C₀-C₆ alkylheteroaryl-, —C₀-C₆-alkylheteroaryl-C₀-C₆-alkyl-, heteroaryl, —C₀-C₆ alkylheteroaryl-C₂-C₆-heteroalkyl-, —C₂-C₆ heteroalkyl-heteroaryl-C₀-C₆-alkyl-, —C₄-C₆ heterocyclyl-heteroaryl-C₀-C₆-alkyl-, —C₀-C₆ alkyl-heteroaryl-C₄-C₆-heterocyclyl-, —C₃-C₆ alkynyl-aryl-C₀-C₆-alkyl, —C₀-C₆ alkyl-aryl-C₃-C₆-alkynyl, —C₃-C₆ alkynyl-heteroaryl-C₀-C₆-alkyl, —C₀-C₆ alkyl-heteroaryl-C₃-C₆-alkynyl, —C₃-C₆ alkenyl-aryl-C₀-C₆-alkyl, —C₀-C₆ alkyl-aryl-C₃-C₆-alkenyl, —C₃-C₆ alkenyl-heteroaryl-C₀-C₆-alkyl, —C₀-C₆ alkyl-heteroaryl-C₃-C₆-alkenyl, —C₀-C₆ alkylaryl-aryl-, —C₀-C₆ alkylaryl-aryl-C₀-C₆-alkyl-, —C₀-C₆ alkylaryl-heteroaryl-, —C₀-C₆ alkylaryl-heteroaryl-C₀-C₆-alkyl-, —C₀-C₆ alkyl-C₃-C₆ cycloalkyl-, —C₀-C₆ alkyl-C₃-C₆ cycloalkyl-C₀-C₆-alkyl-, —C₁-C₆ alkyl-X—C₃-C₆ cycloalkyl-, —C₁-C₆ alkyl-X—C₃-C₆ cycloalkyl-C₀-C₆-alkyl-, —C₁-C₆ alkyl-N(R₃)—C₁-C₆ alkyl-C₃-C₆ cycloalkyl-, —C₁-C₆ alkyl-N(R₃)—C₁-C₆ alkyl-C₃-C₆ cycloalkyl-C₀-C₆-alkyl-, and —C₁-C₆ alkyl-S—S—C₁-C₆ alkyl-, wherein each alkyl, alkenyl, alkynyl, heteroalkyl, aryl, heteroaryl, heterocyclyl, and cycloalkyl moiety is optionally substituted;
L is selected from the group consisting of a covalent bond, —C₀-C₆alkyl-aryl-C₀-C₃alkyl-X—C₀-C₃alkyl, C₀-C₆alkyl-heteroaryl-C₀-C₃alkyl-X—C₀-C₃alkyl, C₀-C₃alkyl-X—C₀-C₃alkyl, C₀-C₆alkyl-N(R₃)—C(O)—N(R₃)—S(O)₂—C₀-C₃alkyl-aryl, —C₀-C₃alkyl-S(O)₂—N(R₃)—C₀-C₃alkyl-aryl-C₀-C₃alkyl-C(O)—N(R₃)—C₀-C₃alkyl, —C₀-C₃alkyl-N(R₃)—C(O)—O-heterocyclyl-C₀-C₃alkyl, C₀-C₃alkyl-heteroaryl-C₀-C₃alkyl-N(R₃)—C₀-C₃alkyl, —C₀-C₆ alkyl-N(R₃)C(O)heterocyclyl-C₀-C₃alkyl, —C₀-C₆ alkyl-S(O)₂heterocyclyl-C₀-C₃alkyl-, —C₀-C₆ alkyl-N(R₃)S(O)₂heterocyclyl-C₀-C₃alkyl, —C₀-C₆ alkyl-, —C₂-C₆ alkenyl-, —C₂-C₆ alkynyl-, —C₀-C₆ alkyl-N(R₃)C(O)—C₀-C₃ alkyl, —C—C₆ alkyl-N(R₃)C(S)—C₀-C₃ alkyl, —C₀-C₆ alkyl-C(O)N(R₃)C(O)—C₀-C₃ alkyl, —C₂-C₆ heteroalkyl-N(R₃)C(O)—C₀-C₃ alkyl, —C₀-C₆ alkyl-C(O)—N(R₃)—C₀-C₃ alkyl, —C₀-C₆ alkyl-C(S)—N(R₃)—C₀-C₃ alkyl, —C₀-C₆ alkyl-OC(O)—, —C₀-C₆ alkyl-C(O)—O—, —C₀-C₆ alkyl-C(O)—C₀-C₃ alkyl, —C₀-C₆ alkyl-SO₂—N(R₃)—C₀-C₃ alkyl, —C₀-C₆ alkyl-N(R₃)—SO₂—C₀-C₃ alkyl, —C₀-C₆ alkyl-C(O)—N(R₃)—SO₂—C₀-C₃ alkyl, —C₀-C₆ alkyl-C(O)—N(R₃)—SO₂—C₀-C₃ alkyl-aryl, —C₀-C₆ alkyl-C(O)—N(R₃)—SO₂—C₀-C₃ alkyl-heteroaryl, —C₀-C₆ alkyl-N(R₃)—C₀-C₃ alkyl, —C₀-C₆ alkyl-N(R₇)—C₀-C₃ alkyl, —C₀-C₆ alkyl-S—C₀-C₃ alkyl, —C₀-C₆ alkyl-O—C₀-C₃ alkyl-, —C₀-C₆ alkyl-S(O)—C₀-C₃ alkyl, —C₀-C₆ alkyl-S(O)₂—C₀-C₃ alkyl, —C₀-C₆ alkyl-(CR₃═CR₃)₁₋₂—C₁-C₆ alkyl-, —C₀-C₆ alkyl-(C≡C)₁₋₂—C₁-C₆ alkyl-, —C₀-C₆ alkyl-N(R₃)—C(O)—N(R₃)—C₀-C₃ alkyl, —C₀-C₆ alkyl-N(R₃)—C(O)—O—C₀-C₃ alkyl, —C₀-C₆ alkyl-O—C(O)—N(R₃)—C₀-C₃ alkyl, —C₀-C₃ alkyl N(R₃)C(O)—C₀-C₃ alkyl-heterocyclyl-C(O)—C₀-C₆ alkyl, —C₀-C₃ alkyl-heterocyclyl-C₀-C₃ alkyl, —C₀-C₃ alkyl-heterocyclyl-C₂-C₄ alkenyl-C₀-C₃ alkyl, —C₀-C₃ alkyl-heterocyclyl-C₀-C₃ alkyl-O—C₀-C₃ alkyl, —C₀-C₃ alkyl-O—C₀-C₃ alkyl heterocyclyl-C₀-C₃ alkyl, —C₀-C₃ alkyl-heterocyclyl-C₀-C₃ alkyl-N(R₃)—C₀-C₃ alkyl, —C₀-C₃ alkyl-N(R₃)—C₀-C₃ alkyl heterocyclyl-C₀-C₃ alkyl, —C₀-C₃ alkyl-heterocyclyl-C₀-C₃ alkyl-S—, —C₀-C₃ alkyl S(O)₂N(R₃)—C₀-C₃ alkyl heterocyclyl-C₀-C₃ alkyl, —C₀-C₃ alkyl S(O)₂-heterocyclyl-C₀-C₃ alkyl, —C₀-C₃ alkyl-C(O)N(R₃)—C₀-C₃ alkyl-heterocyclyl-C₀-C₃ alkyl, —C₀-C₃ alkyl-C(S)N(R₃)—C₀-C₃ alkyl-heterocyclyl-C₀-C₃ alkyl, —C₀-C₃ alkyl C(O)-heterocyclyl-C₀-C₃ alkyl, —C₀-C₃ alkyl OC(O)N(R₃)—C₀-C₃ alkyl-heterocyclyl-C₀-C₃ alkyl, —C₀-C₃ alkyl-OC(S)N(R₃)—C₀-C₃ alkyl-heterocyclyl-C₀-C₃ alkyl, —C₀-C₃ alkyl-OC(O)-heterocyclyl-C₀-C₃ alkyl, —C₀-C₃ alkyl N(R₃)C(O)N(R₃)—C₀-C₃ alkyl-heterocyclyl-C₀-C₃ alkyl, —C₀-C₃ alkyl N(R₃)C(S)N(R₃)—C₀-C₃ alkyl-heterocyclyl-C₀-C₃ alkyl, —C₀-C₃ alkyl N(R₃)C(O)-heterocyclyl-C₀-C₃alkyl, —C₀-C₃ alkyl N(R₃)C(S)-heterocyclyl-C₀-C₃ alkyl, —C₀-C₃ alkyl N(R₃)S(O)₂N(R₃)—C₀-C₃ alkyl-heterocyclyl-C₀-C₃ alkyl, —C₀-C₃ alkyl-C═N—O—C₀-C₃ alkyl, —C₀-C₃ alkyl N(R₃)C(O)—C₁-C₃ alkyl-N(R₃)C(O)—C₀-C₃ alkyl, —C₀-C₃ alkyl N(R₃)C(S)—C₁-C₃ alkyl-N(R₃)C(S)—C₀-C₃ alkyl, —S(O)₂—N(R₃)—C₀-C₃ alkyl-aryl-C₀-C₃ alkyl-N(R₃)C(O)—C₁-C₃ alkyl-, —S(O)₂—N(R₃)—C₀-C₃ alkyl-aryl-C₀-C₃ alkyl-N(R₃)C(S)—C₁-C₃ alkyl-, —N(R₃)—S(O)₂—C₀-C₃ alkyl-aryl-C₀-C₃ alkyl-N(R₃)C(O)—C₁-C₃ alkyl-, —N(R₃)—S(O)₂—C₀-C₃ alkyl-aryl-C₀-C₃ alkyl-N(R₃)C(S)—C₁-C₃ alkyl-, —S(O)₂—N(R₃)—C₀-C₃ alkyl-heteroaryl-C₀-C₃ alkyl-N(R₃)C(O)—C₁-C₃ alkyl-, —S(O)₂—N(R₃)—C₀-C₃ alkyl-heteroaryl-C₀-C₃ alkyl-N(R₃)C(S)—C₁-C₃ alkyl-, —N(R₃)—S(O)₂—C₀-C₃ alkyl-heteroaryl-C₀-C₃ alkyl-N(R₃)C(O)—C₁-C₃ alkyl- and —N(R₃)—S(O)₂—C₀-C₃ alkyl-heteroaryl-C₀-C₃ alkyl-N(R₃)C(S)—C₁-C₃ alkyl-, wherein each alkyl, alkenyl, alkynyl, heteroalkyl, cycloalkyl, heterocycloalkyl, aryl and heteroaryl moiety of the aforementioned L are optionally substituted,
—(C₀-C₃alkyl)(R₃)N—S(O)₂—N(R₃)—C₂-C₄alkyl-O—C₀-C₃alkyl, when Y is absent,
—R₃R₃ₐNS(O)₂N(R₃)—C₂-C₄alkyl-O—C₀-C₆ alkyl-, when Y is absent, and
—R₃R₃ₐNS(O)₂N(R₃)—C₂-C₄alkyl, when Y is absent,
wherein the right end attaches to Z and the left end attaches to Y;
Y is selected from the group consisting of alkyl, heteroalkyl, cycloalkyl, heterocyclyl, alkylcycloalkyl, alkylheterocyclyl, aryl, alkylaryl, heteroaryl, alkylheteroaryl, aryl-heteroaryl, alkylaryl-heteroaryl, heteroaryl-alkylaryl, aryl-aryl, alkylaryl-aryl, aryl-alkylaryl, heteroaryl-heteroaryl, heteroaryl-aryl, alkylheteroaryl-aryl, aryl-alkylheteroaryl, heteroaryl-aryl-aryl, aryl-aryl-heteroaryl, alkylheteroaryl-aryl-aryl, aryl-aryl-alkylheteroaryl, heteroaryl-aryl-heteroaryl, alkylheteroaryl-aryl-heteroaryl, heteroaryl-aryl-alkylheteroaryl, alkylheteroaryl-heteroaryl, heteroaryl-alkylheteroaryl, heterocycyl-heteroaryl, heteroaryl-heterocyclyl, heterocyclyl-aryl, aryl-heterocyclyl, heterocyclyl-alyl-aryl, aryl-alkyl-heterocyclyl, aryl-C₁-C₃alkyl-aryl, —(O)C—C₀-C₃alkyl-aryl, C₀-C₃alkyl-aryl, —C₀-C₃alkyl-aryl-O—C₂-C₄alkyl-N(R₃)(R₃ₐ), —C₀-C₃alkyl-heteroaryl-O—C₂-C₄alkyl-N(R₃)(R₃ₐ), —C₀-C₃alkyl-aryl-C₀-C₃alkyl, C₀-C₃alkyl-heteroaryl-C₀-C₃alkyl and aryl-C₁-C₃alkyl-heteroaryl, each optionally substituted with one or more groups selected from R₃, R₄ or R₇; or
Y-L-Z- is selected from the group consisting of aryl-C₂-C₆ alkynyl-C₁-C₄alkyl, heteroaryl-C₂-C₆-alkynyl-C₁-C₄alkyl, R₃-heterocyclyl-C₀-C₃ alkyl-NR₃C(O)NR₃-heteroaryl-C₂-C₇alkyl; R₃-heterocyclyl-C₀-C₃ alkyl-NR₃C(O)NR₃-aryl-C₂-C₇alkyl; aryl-C₀-C₆ alkyl-, heteroaryl-C₁-C₆ alkyl-N(R₄)—C₁-C₆-alkyl-aryl-C₀-C₆ alkyl-, heteroaryl-C₀-C₆ alkyl-heteroaryl-C₀-C₇ alkyl-aryl-C₀-C₆-alkyl, heteroaryl-C₀-C₆ alkyl-, aryl-C₀-C₆ alkyl-heteroaryl-C₀-C₆ alkyl-, aryl-C₀-C₆ alkyl-aryl-C₀-C₆ alkyl-, aryl-C₀-C₆ alkyl-aryl-C₀-C₆ alkenyl-, aryl-C₀-C₆ alkyl-aryl-C₀-C₆ alkynyl-, aryl-C₀-C₆ alkenyl-aryl-C₀-C₆ alkyl-, aryl-C₀-C₆ alkenyl-aryl-C₀-C₆ alkenyl-, aryl-C₀-C₆ alkenyl-aryl-C₀-C₆ alkynyl-, aryl-C₀-C₆ alkynyl-aryl-C₀-C₆ alkyl-, aryl-C₀-C₆ alkynyl-aryl-C₀-C₆ alkenyl-, aryl-C₀-C₆ alkynyl-aryl-C₀-C₆ alkynyl-, heteroaryl-C₀-C₆ alkyl-aryl-C₀-C₆ alkyl-, heteroaryl-C₀-C₆ alkyl-aryl-C₀-C₆ alkenyl-, heteroaryl-C₀-C₆ alkyl-aryl-C₀-C₆ alkynyl-, heteroaryl-C₀-C₆ alkenyl-aryl-C₀-C₆ alkyl-, heteroaryl-C₀-C₆ alkenyl-aryl-C₀-C₆ alkenyl-, heteroaryl-C₀-C₆ alkenyl-aryl-C₀-C₆ alkynyl-, heteroaryl-C₀-C₆ alkynyl-aryl-C₀-C₆ alkyl-, heteroaryl-C₀-C₆ alkynyl-aryl-C₀-C₆ alkenyl-, heteroaryl-C₀-C₆ alkynyl-aryl-C₀-C₆ alkynyl-, aryl-C₀-C₆ alkyl-heteroaryl-C₀-C₆ alkyl-, aryl-C₀-C₆ alkyl-heteroaryl-C₀-C₆ alkenyl-, aryl-C₀-C₆ alkyl-heteroaryl-C₀-C₆ alkynyl-, aryl-C₀-C₆ alkenyl-heteroaryl-C₀-C₆ alkyl-, aryl-C₀-C₆ alkenyl-heteroaryl-C₀-C₆ alkenyl-, aryl-C₀-C₆ alkenyl-heteroaryl-C₀-C₆ alkynyl-, aryl-C₀-C₆ alkynyl-heteroaryl-C₀-C₆ alkyl-, aryl-C₀-C₆ alkynyl-heteroaryl-C₀-C₆ alkenyl-, aryl-C₀-C₆ alkynyl-heteroaryl-C₀-C₆ alkynyl-, heteroaryl-C₀-C₆ alkyl-heteroaryl-C₀-C₆ alkyl-, heteroaryl-C₀-C₆ alkyl-heteroaryl-C₀-C₆ alkenyl-, heteroaryl-C₀-C₆ alkyl-heteroaryl-C₀-C₆ alkynyl-, heteroaryl-C₀-C₆ alkenyl-heteroaryl-C₀-C₆ alkyl-, heteroaryl-C₀-C₆ alkenyl-heteroaryl-C₀-C₆ alkenyl-, heteroaryl-C₀-C₆ alkenyl-heteroaryl-C₀-C₆ alkynyl-, heteroaryl-C₀-C₆ alkynyl-heteroaryl-C₀-C₆ alkyl-, heteroaryl-C₀-C₆ alkynyl-heteroaryl-C₀-C₆ alkenyl-, heteroaryl-C₀-C₆ alkynyl-heteroaryl-C₀-C₆ alkynyl-, heteroaryl-C₀-C₆ alkyl-heteroaryl-C₀-C₇ alkyl-aryl-, heteroaryl-C₀-C₆ alkyl-heteroaryl-C₀-C₇ alkyl-aryl-C₀-C₃-alkyl, aryl-C₀-C₆ alkyl-heteroaryl-C₀-C₇ alkyl-aryl-, aryl-C₀-C₆ alkyl-heteroaryl-C₀-C₇ alkyl-aryl-C₀-C₃-alkyl, aryl-C₀-C₆ alkyl-heteroaryl-C₀-C₇ alkyl-heteroaryl-, aryl-C₀-C₆ alkyl-heteroaryl-C₀-C₇ alkyl-heteroaryl-C₀-C₃-alkyl, heteroaryl-C₀-C₆ alkyl-heteroaryl-C₀-C₇ alkyl-aryl-, heteroaryl-C₀-C₆ alkyl-heteroaryl-C₀-C₇ alkyl-aryl-C₀-C₃-alkyl, heteroaryl-C₀-C₆ alkyl-heteroaryl-C₀-C₇ alkyl-, heteroaryl-C₀-C₆ alkyl-aryl-C₀-C₇ alkyl-, heteroaryl-C(O)—C₀-C₆ alkyl-heteroaryl-C₀-C₇ alkyl-, heteroaryl-C(O)—C₀-C₆ alkyl-aryl-C₀-C₇ alkyl-, heteroaryl-S(O)₂—C₀-C₆ alkyl-heteroaryl-, heteroaryl-C₀-C₆ alkyl-N(R₃)—C(O)-C₁-C₇ alkyl-, aryl-C₀-C₆ alkyl-C(O)—N(R₃)—C₁-C₇ alkyl-, heteroaryl-C₀-C₆ alkyl-C(O)—N(R₃)—C₁-C₇ alkyl-, C₁-C₆ alkyl-C(O)—N(R₃)—C₁-C₇ alkyl-, heterocyclyl-C₀-C₆ alkyl-C(O)—N(R₃)—C₁-C₇ alkyl-, C₁-C₆ cycloalkyl-C(O)—N(R₃)—C₁-C₇ alkyl-, (R₃)(R₃ₐ)N-C₀-C₆alkyl-C(O)—N(R₃)—C₁-C₇alkyl, aryl-C₀-C₆alkyl-O—C(O)—N(R₃)—C₁-C₇alkyl, aryl-C₁-C₃ alkyl-N(R₃)—C(O)—C₁-C₇ alkyl-, C₁-C₃ alkyl-N(R₃)—C(O)—C₁-C₇ alkyl-, heteroaryl-S(O)₂—C₀-C₆ alkyl-aryl-, aryl-C₀-C₆ alkyl-N(R₃)—C(O)—C₁-C₇ alkyl-, —R₃—O—C(O)NR₃—C₀-C₃alkyl-heteroaryl-C₁-C₇alkyl-, R₃—C(O)—C₀-C₃alkyl-heteroaryl-C₁-C₇alkyl-, R₃—C(O)-heterocyclyl-C₀-C₃alkyl-heteroaryl-C₀-C₇alkyl-, R₃-heterocyclyl-C₀-C₃alkyl-N(R₃)C(O)N(R₃)—C₀-C₃alkyl-heteroaryl-C₀-C₇alkyl-, R₃-heterocyclyl-C₀-C₃alkyl-N(R₃)C(O)—C₀-C₃alkyl-heteroaryl-C₀-C₇alkyl- and R₃-heterocyclyl-C₀-C₃alkyl-N(R₃)S(O)₂—C₀-C₃alkyl-heteroaryl-C₀-C₇alkyl-, wherein the alkyl, alkenyl, alkynyl, heteroalkyl, cycloalkyl, aryl, heterocyclyl and heteroaryl moieties of the aforementioned Y-L-Z are optionally substituted,
A₂ₐ-aryl-C₀-C₃ alkyl-N(R₃)—C(O)—C₁-C₇ alkyl-, wherein the C₁-C₇ alkyl is optionally substituted with —NR₃—C(O)—C₁-C₅ alkyl-C₂-C₄ alkenyl-C₁-C₃ alkyl-O-A_{2b}, or —N(R₃)—C(O)—C₁-C₇alkyl-O-A_{2b},
A₂ₐ-heteroarylene-C₀-C₃ alkyl-N(R₃)—C(O)—C₁-C₇ alkyl-, wherein the C₁-C₇ alkyl is optionally substituted with —NR₃—C(O)—C₁-C₅ alkyl-C₂-C₄ alkenyl-C₁-C₃ alkyl-O-A_{2b},
A₁ₐ-O-aryl-C₀-C₃alkyl-N(R₃)—C(O)—C₁-C₇alkyl, wherein the C₁-C₇ alkyl is optionally substituted with —N(R₃)—C(O)—C₁-C₇alkyl-A_{1b}, and
B₂—B₁—N(R₃)—C(O)—C₁-C₇ alkyl-, wherein the C₁-C₇ alkyl is optionally substituted with —NR₃—B₃ and the amine of B₃ is conected with the acid of B₂ to form a peptide bond; wherein
A₁ₐ and A_{1b} are independently selected from the group consisting of alkyl, alkenyl and a protecting group; or
A₁ₐ and A_{1b} together via a —C₂-C₆alkylene, —C₂-C₆alkenylene or —C₂-C₆alkynylene linker, form an optionally substituted ring;
A₂ₐ and A_{2b} together are a covalent bond and are attached to form a ring; and
B₁, B₂ and B₃ are each independently a natural or synthetic amino acid; or
L is a covalent bond and Z is C₀-C₆alkyl, heteroalkyl, —C₀-C₆ alkyl-heterocyclyl-C₀-C₆ alkyl-, -heterocyclyl-C(O)—C₂-C₆ alkenyl-C₁-C₃ alkyl-, —C₀-C₇ alkyl-N(R₃)—C(O)-heterocyclyl-C₀-C₇ alkyl-, —C₀-C₇ alkyl-N(R₃)—C(S)-heterocyclyl-C₀-C₇ alkyl-, —C₀-C₇ alkyl-,O—C(O)-heterocyclyl-C₀-C₆ alkyl-, —C₀-C₇ alkyl-O—C(S)-heterocyclyl-C₀-C₆ alkyl-, —C₀-C₆ alkyl-C(O)-heterocyclyl-C₀-C₆ alkyl-, —C₀-C₆ alkyl-C(S)-heterocyclyl-C₀-C₆ alkyl-, —C₀-C₆ alkyl-S(O)₂-heterocyclyl-C₀-C₆ alkyl-, —C₀-C₆ alkyl-heterocyclyl-C(O)—C₀-C₆ alkyl-, —C₀-C₆ alkyl-heterocyclyl-C(S)—C₀-C₆ alkyl-, —C₀-C₆ alkyl-heterocyclyl-S(O)₂—C₀-C₆ alkyl-, —C₀-C₆ alkyl-heterocyclyl-N(R₃)C(O)—C₀-C₆ alkyl-, —C₀-C₆ alkyl-heterocyclyl-O—C(O)—C₀-C₆ alkyl-, —C₀-C₆ alkyl-heterocyclyl-N(R₃)C(S)—C₀-C₆ alkyl-, —C₀-C₆ alkyl-heterocyclyl-O—C(S)—C₀-C₆ alkyl-, and —X—C₁-C₆ alkyl-C(R₃)═C(R₃)—C₁-C₆ alkyl-, wherein each alkyl, alkenyl and heterocyclyl of the aforementioned Z is optionally substituted;
R₆ is selected from the group consisting of —H, C₁-C₆ alkyl, C₁-C₆ alkenyl, C₁-C₆ alkynyl, C₁-C₆ heteroalkyl, heterocyclyl-C₀-C₆ alkyl-, aryl-C₀-C₆ alkyl-, heteroaryl-C₀-C₆ alkyl-, C₃-C₆ cycloalkyl-C₀-C₆ alkyl-, N(R₃)(R₃ₐ)-C₁-C₆ alkyl- and N(R₃)(R₃ₐ)—C(O)—C₁-C₆ alkyl-, wherein each alkyl, alkenyl, alkynyl, heteoralkyl, cycloalkyl, aryl, heteroaryl, or heterocyclyl moiety is optionally substituted; and
R₇ and R₇ₐ are independently selected from the group consisting of —H, C₁-C₆ alkyl-, C₁-C₆ alkenyl, C₁-C₆ alkynyl, C₁-C₆ heteroalkyl, R₃—O—C₁-C₆ alkyl-, N(R₃)(R₃ₐ)-C₁-C₆ alkyl-, a protecting group, —C(O)—O—C₁-C₆ alkyl, —C(O)—O-benzyl and heterocyclyl-C₁-C₆ alkyl-, wherein each alkyl, alkenyl, alkynyl, heteroalkyl, benzyl and heterocyclyl moiety is independently optionally substituted; or
R₇ is —OR₃ when attached to the N atom of an indolyl moiety;
wherein in a —N(R₃)(R₃ₐ) group, optionally the R₃ and R₃ₐ together with the nitrogen atom to which they are attached form a heterocyclyl group; or
wherein in a —N(R₄)(R₄ₐ) group, optionally the R₄ and R₄ₐ together with the nitrogen atom to which they are attached form a heterocyclyl group; and
provided that when R₃, R₃ₐ, R₄ and R₄ₐ are present in —N(R₃)(R₃ₐ), —N(R₄)(R₄ₐ), —NR₃, —OR₃, —SR₃, -alkyl-R₃, —NR₃S(O)₂R₃, —S(O)CH₂R₃, —NR₃S(O)₂CH₂R₃, —NR₃C(O)CH₂R₃(C═NR₃)N(R₃)(R₃ₐ), —C(O)R₃, —NR₄ and —CR₃═CR₃, then R₃, R₃ₐ, R₄ and R₄ₐ are independently H, —C₁-C₆-alkyl, —C₃-C₆-cycloalkyl, heteroalkyl, aryl, alkyl-aryl, heteroaryl or alkyl-heteroary;
when Y-L-Z- is phenyl, W is nitrogen, X is a covalent bond or —CH₂—, R₁ and R₂ are —H, and Q is —C₁-C₆-alkyl-C(O)—OR₃, then R₃ is not —H;
when Y-L-Z- is phenyl, W is nitrogen, X is a covalent bond or —CH₂—, R₁ and R₂ are —H, and Q is —C₁-C₆-alkyl-N(R₃)(R₃ₐ), and R₃ is —H, then R₃ₐ is not —H;
when Y-L-Z- is phenyl, W is nitrogen, X is a covalent bond or —CH₂—, R₁ and R₂ are —H, Q is —C₁-C₆ alkyl-C(O)—N(R₃)(R₃ₐ), and R₃ is —H, then R₃ₐ is not —H, —OH, or phenyl substituted with —NH₂ or —OH;
when Y-L-Z- is phenyl or phenyl-CH₂—, W is nitrogen, X is a covalent bond or —CH₂—, R₁ and R₂ are —H, and Q is —C₁-C₆ alkyl-N(H)—S(O)₂—R₃, then R₃ is not —CH₃;
when Y-L-Z- is aryl-C₁-C₆ alkyl- or heteroaryl-C₁-C₆ alkyl-, W is nitrogen, X is a covalent bond or —CH₂—, R₁ and R₂ are —H, Q is —C₁-C₆ alkyl-C(O)—N(R₃)(R₃ₐ), and R₃ is —H, then R₃ₐ is not —OH;
when Y-L-Z- is aryl-C₁-C₆ alkyl-, aryl, cycloalkyl, heterocyclyl, heteroaryl, or heteroaryl-C₁-C₆ alkyl-, W is nitrogen, X is a covalent bond or —CH₂—, R₁, R₂ and R₃ are —H, Q is not —C₁-C₆ alkyl-N(H)—C(O)—CH₂—SH;
when Y-L-Z- is aryl-C₁-C₆ alkyl-, aryl, or heteroaryl, W is nitrogen, R₁, R₂ and R₃ are —H, —X-Q is not —C₁-C₆ alkyl-SH;
when Y-L-Z- is phenyl optionally para substituted with —N(CH₃)₂, naphthyl, indolyl, or benzofuranyl, W is nitrogen, X is a covalent bond or —CH₂—, R₁, R₂ and R₃ are —H, then Q is not —C₁-C₆alkyl-OH;
when Y-L-Z- is phenyl optionally para substituted with —N(CH₃)₂, quinolinyl, biphenyl or benzyl, W is nitrogen, X is a covalent bond or —CH₂—, R₁, R₂ and R₃ are —H, then Q is not —C₁-C₆ alkyl-N(H)—S(O)₂—CH₃, —C₁-C₆ alkyl-S(O)₂—N(H)—OH, —C₁-C₆ alkyl-N(H)—C(O)—NH₂, —C₁-C₆ alkyl-N(H)—C(O)—C₁-C₂ alkyl-SH, —C₁-C₆alkyl-C(O)—N(H)—OH, —C₁-C₆alkyl-C(O)—OH, or —C₁-C₆ alkyl-N(H)—C(O)—O-t-butyl;
when Y-L-Z- is phenyl optionally para substituted with —N(CH₃)₂, biphenyl, substituted pyrrolyl, or substituted pyrrolidinyl, W is nitrogen, X is a covalent bond or —CH₂—, R₁, R₂ and R₃ are —H, then Q is not —C₁-C₆alkyl-C(O)—N(H)—OH;
when Y-L-Z- is phenyl, benzyl, or quinolinyl, W is nitrogen, X is a covalent bond or —CH₂—, R₁ is —H, R₂ is —N(H)—C(O)—O-t-butyl or —N(H)—C(O)—O-benzyl, then Q is not —C₁-C₆alkyl-C(O)—N(H)—OH, —C₁-C₆ alkyl-imidazolyl, —C₁-C₆ alkyl-SO₂—NH₂, —C₁-C₆ alkyl-C(O)—N(H)-imidazolyl, —C₁-C₆ alkyl-C(O)—N(H)-thiazolyl, —C₁-C₆ alkyl-C(O)—N(H)-pyridinyl, —C₁-C₆ alkyl-C(O)—N(H)-anilinyl, —C₁-C₆ alkyl-NH₂, —C₁-C₆ alkyl-N(H)—S(O)₂—CH₃, or —C₁-C₆ alkyl-C(O)—O—R₃, wherein R₃ is —CH₃, -t-butyl, or —H;
when Y-L-Z- is phenyl, W is nitrogen, X is a covalent bond or —CH₂—, R₁, R₂ and R₃ are —H, then Q is not C₁-C₆-alkyl-NH—S(O)₂—CH₃, C₁-C₆-alkyl-S(O)₂—NH—OH, C₁-C₆-alkyl-NH—C(O)—NH₂, —C₁-C₆-alkyl-NH₂, —C₁-C₆-alkyl-NH—C(O)—C₁-C₂-alkyl-halo, —C₁-C₆-alkyl-NH—C(O)—C₁-C₂-alkyl-NH₂ or —C₁-C₆-alkyl-NH—C(O)—CH₂—OH; or
when Y is phenyl optionally para substituted with —N(CH₃)₂, L is —C(O)—NH—CH₂—, Z is phenyl-CH₂, W is N, R₁, R₂ and R₃ are —H, and X is a covalent bond, Q is not —SH; and
further provided that Formula (I) excludes those compounds wherein
X is S;
Q is selected from the group consisting of H, methyl, ethyl, phenyl, benzyl and acetyl; and
Y-L-Z is selected from the group consisting of R^{a}—(CH₂)₄₋₆ and R^{b}—Ar—(CH₂)₁₋₂—, wherein
R^{a} is selected from the group consisting of R^{c}NR^{d}C(O)—, R^{c}NHC(O)NH—, R^{c}NHC(S)NH—, R^{c}SO₂NH— and R^{c}C(O)NH—;
R^{b} is selected from the group consisting of R^{c}NR^{d}C(O)(CH₂)₁₋₂—, R^{c}NHC(O)NH(CH₂)₁₋₂—, R^{c}NHC(S)NH(CH₂)₁₋₂, R^{c}SO₂NH(CH₂)₁₋₂— and R^{c}C(O)NH(CH₂)₁₋₂—;
R^{c} is selected from the group consisting of C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀— alkyl-heteroaryl, -aryl-OCH₂-aryl, -aryl-CH₂O-aryl, -aryl-carbonyl-aryl, -aryl-C(O)CH₃, -aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl and —CH(aryl)(aryl), any of which is optionally substituted with one or more of R^{e} or R^{f};
R^{e} or R^{f} are C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl or heterocyclyl; and
R^{d} is C₀₋₁alkyl, or R^{c} and R^{d} taken together form a heterocyclic or carbocyclic ring, any of which is optionally substituted with one or more independent C₀₋₄alkyl, halogen, —OH, —SCH₃, OCH₃, —NH₂, aryl, or heterocyclyl substituents; and
Ar is aryl optionally substituted with one or more independent C₁₋₄alkyl, halogen, —OH, —SCH₃, —OCH₃, —NH₂, aryl or heterocyclyl substituents, and
further provided that Formula (I) excludes those compounds wherein 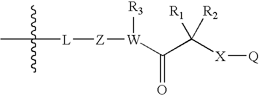
is —(CH₂)₃₋₄—NH(CO)—CH₂—O—CH₂-phenyl or —(CH₂)₃₋₄—NHC(O)—CH₂—S-phenyl and
Y is selected from the group consisting of optionally substituted imidazopyridinyl or optionally substituted imidazonaphthyridine; and
further provided that Formula (I) excludes those compounds wherein 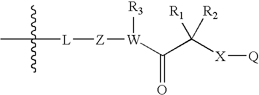
is —(CH₂)₂₋₃—NHC(O)—CH₂—O—CH₂-phenyl or —(CH₂)₂₋₃—NHC(O)—CH₂—S-phenyl, wherein the phenyl is optionally substituted with halogen, and Y is dimethyoxyphenyl; and
further provided that Formula (I) excludes those compounds wherein 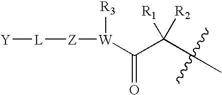
is 3-(R^{t})(R^{tt})CH-phenyl-1-O—C₃-C₄alkyl-NHC(O)—, 3-(R^{t})(R^{tt})CH-phenyl-1-O—C₃-C₄alkenyl-NHC(O)—, (R^{t})(R^{tt})CH-thiophene-O—C₃-C₄alkyl-NHC(O)—, (R^{t})(R^{tt})CH-thiophene-O—C₃-C₄alkenyl-NHC(O)—, (R^{t})(R^{tt})CH-pyridine-O—C₃-C₄alkyl-NHC(O)— and (R^{t})(R^{tt})CH-pyridinyl-O—C₃-C₄alkenyl-NHC(O)—, wherein R^{t} is selected from the group consisting of H, halogen, OH, Me, optionally substituted piperidino, dimethylamino, 1-pyrrolidinyl and 1-perhydroazepinyl, and R^{tt} is H or Me, or R^{t} is oxo and R^{tt} is absent, with the exception the this proviso does not include the compound 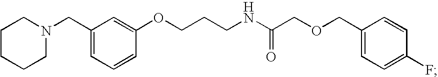
and
further provided that Formula (I) excludes indol-(CH₂)₂—NHC(O)—CH₂—O—CH₂-phenyl, indol-(CH₂)₂—NHC(O)—CH₂—S(O)₂-phenyl-Me, phenyl-(CH₂)₂—NHC(O)—CH₂—S(O)₂-phenyl, phenyl-(CH₂)₂—NHC(O)—CH₂—S(O)₂-phenyl-Me, T-(CH₂)₂₋₅—NHC(O)—CH₂—S-phenyl (wherein T is pheny, fluro-phenyl, pyridine, methyl-pyrrolidine or methyl), NH₂—S(O)₂-phenyl-(CH₂)₂—NHC(O)—CH₂—S-phenyl, CH₃—(CH₂)₂—NHC(O)—CH₂—O—CH₂-phenyl, CH₃—(CH₂)₂—NHC(O)—CH₂—S(O)₂-phenyl, CH₃—(CH₂)₂—NHC(O)—CH₂—S-phenyl, N-[[[[(aryloxy- or -thio)alkyl]carbonyl]amino]alkyl]-2,2,5,5-tetramethyl-3-pyrroline-3-carboxamide, 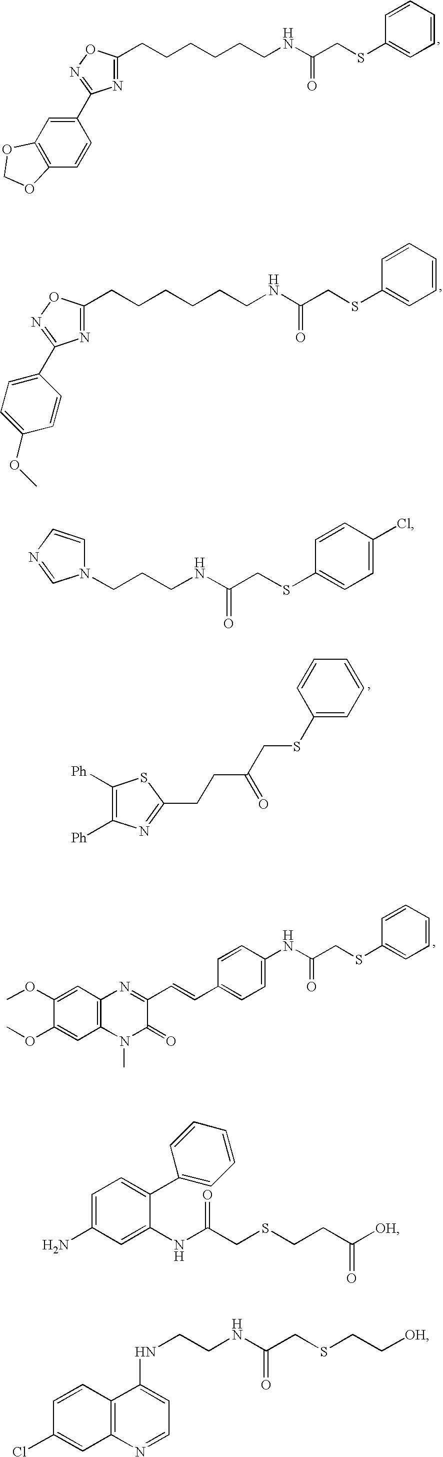 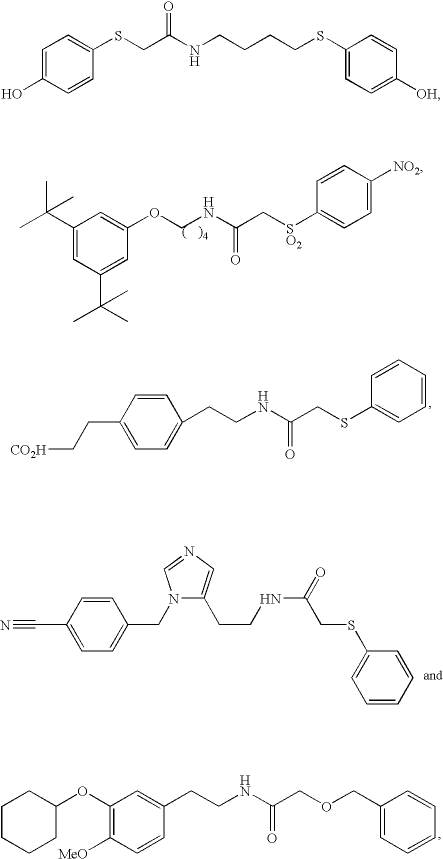
and
further provided that Formula (I) excludes compounds of formula (R^{v})(R^{vv})pyrimidine-NHS(O)₂-phenyl-C₀-C₄alkyl-NHC(O)-A, wherein A is aryloxylalkyl or arylmercaptoalkyl, R^{v} is a lower alkyl, and R^{vv} is selected from the group consisting of H, unsubstituted or substituted alkyl, cycloalkyl, aryl, aralkyl, alkoxy, alkoxyalkyl and alkoxyalkoxy, or wherein R^{v} and R^{vv} taken together form a ring of 3 to 5 methylene groups which can contain oxygen or sulfur atoms.

2. The compound according to claim 1, wherein each alkyl, alkenyl, alkynyl, heteroalkyl, cycloalkyl, heterocyclyl, aryl and heteroaryl moiety of X-Q, Q, L, Z, R³ and R^{3a} is independently optionally substituted with one or more groups independently selected from R⁴.

3. The compound according to claim 2, wherein each alkyl, alkenyl, alkynyl, heteroalkyl, cycloalkyl, heterocyclyl, aryl and heteroaryl moiety of X-Q, Q, R³ and R^{3a} is independently optionally substituted with one or more groups independently selected from oxo, —OH, —CN, C₁-C₆ alkyl, C₁-C₆ alkoxy, —NO₂, —N(R₄)(R₄ₐ), halo, —SH, —S—C₁-C₆ alkyl, —S(O)—C₁-C₆ alkyl, —S—C(O)—C₁-C₆ alkyl and mono- to per-halogenated C₁-C₆ alkyl.

4. The compound according to claim 1, wherein C₁-C₆ alkyl of R₄ and R₄ₐ is optionally substituted with —OH, —NO₂ or C₀-C₆ alkyl-C(O)—N(R₃)(R₃ₐ).

5. The compound according to claim 1, wherein each alkyl, alkenyl, alkynyl, heteroalkyl, aryl, heteroaryl, cycloalkyl and heterocyclyl moiety of Z is independently optionally substituted with one or more groups independently selected from oxo, —OH, —CN, C₁-C₆ alkyl, C₁-C₆ alkoxy, —NO₂, —N(R₃)(R₃ₐ), halo, —SH and mono- to per-halogenated C₁-C₆ alkyl.

6. The compound according to claim 1, wherein L is selected from the group consisting of
—C₁-C₆ alkyl-N(R₃)—C₀-C₃ alkyl wherein the C₁-C₆ alkyl is optionally substituted with —C₁-C₄ alkyl-OR₃, or —C₀-C₄ alkyl-C(O)OR₃,
—C₀-C₆ alkyl-N(R₃)—C(O)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)-,
—C₀-C₆ alkyl-N(R₃)—C(S)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)—,
—C₀-C₆ alkyl-C(O)—N(R₃)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)—,
—C₀-C₆ alkyl-C(S)—N(R₃)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)—, and
—C₀-C₆ alkyl-N(R₃)—C₀-C₃ alkyl- wherein the C₁-C₆ alkyl is optionally substituted with —C₀-C₆ alkyl-O(R₃)—, —C₀-C₆ alkyl-C(O)O(R₃)— or —C₀-C₆ alkyl-N(R₃)(R₃ₐ)—.

7. The compound according to claim 1, wherein each alkyl, alkenyl, alkynyl, heteroalkyl, aryl, heteroaryl, cycloalkyl and heterocyclyl moiety of Y-L-Z is independently optionally substituted with one or more groups independently selected from oxo, —NO₂, C₁-C₆ alkoxy, halo, R₃, R₄ and R₆.

8. The compound according to claim 1, wherein Y-L-Z- is selected from the group consisting of
heteroaryl-C₀-C₆ alkyl-N(R₃)—C(O)—C₁-C₇ alkyl-, wherein the C₁-C₇ alkyl is optionally substituted with —N(R₇)(R₇ₐ) or —N(R₃)—C(O)—C₁-C₆ alkyl-R₃,
aryl-C₀-C₆ alkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl or heteroaryl,
heteroaryl-C₀-C₆ alkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl or heteroaryl,
C₁-C₆ alkyl-C(O)—N(R₃)—C₁-C₇ alkyl-, wherein the C₁-C₇ alkyl is optionally substituted with —N(R₇)(R₇ₐ), aryl-aryl, heteroaryl-heteroaryl, heteroaryl-aryl or heteroaryl,
heterocyclyl-C₀-C₆ alkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl or heteroaryl,
C₁-C₆ cycloalkyl-C(O)—N(R₃)—C₁-C₇ alkyl-, wherein the C₁-C₇ alkyl is optionally substituted with —N(R₇)(R₇ₐ), aryl-aryl, aryl-heteroaryl, heteroaryl-heteroaryl, heteroaryl-aryl, or heteroaryl,
C₁-C₃ alkyl-N(R₃)—C(O)—C₁-C₇ alkyl-, wherein the C₁-C₃ alkyl is optionally substituted with —C(O)NR₃—C₁-C₃ alkyl-A₁ₐ and the C₁-C₇ alkyl is optionally substituted with —NR₃—C(O)O—C₁-C₃ alkyl-A_{1b}, —NR₃—C(O)—C₁-C₃ alkyl-A_{1b}, —NR₃—S(O)₂—C₁-C₃ alkyl-A b, —NR₃—C(O)—NR₃—C₁-C₃ alkyl-A_{1b} or —NR₃—S(O)₂—NR₃-C₁-C₃ alkyl-A_{1b}, and
aryl-C₁-C₃ alkyl-N(R₃)—C(O)—C₁-C₇ alkyl-, wherein the C₁-C₃ alkyl is optionally substituted with —C(O)NR₃—C₁-C₃ alkyl-A₁ₐ and the C₁-C₇ alkyl is optionally substituted with —N(R₃)—C(O)O—C₁-C₃ alkyl-A_{1b}, —N(R₃)—C(O)—C₁-C₃ alkyl-A_{1b}, —NR₃—S(O)₂—C₁-C₃ alkyl-A₁ b, —NR₃—C(O)—NR₃—C₁-C₃ alkyl A_{1b} or —NR₃—S(O)₂—NR₃-C₁-C₃ alkyl-A_{1b}.

9. The compound according to claim 1, wherein B₁, B₂ and B₃ are independently selected from the group consisting of D-Gly, L-Gly, D-Pro, L-Pro, D-Tyr, L-Tyr, D-Tyr(OR₃), L-Tyr(OR₃), D-Phe, L-Phe, D-PheR₄, L-PheR₄, D-Aib, L-Aib, D-Ala, L-Ala, D-ProR₃, L-ProR₃, D-Ile, L-Ile, D-Leu, L-Leu D-PheR₃, L-PheR₃, D-Pip and L-Pip.

10. The compound according to claim 1, wherein each alkyl, alkenyl and heterocyclyl moiety of Y-Z is independently optionally substituted with one or more groups independently selected from R⁴.

11. The compound according to claim 1, wherein each alkyl, alkenyl, alkynyl, heteoralkyl, cycloalkyl, aryl, heteroaryl, and heterocyclyl moiety of R₆ is independently optionally substituted with one or more groups independently selected from R₃ and R₄.

12. The compound according to claim 1, wherein each alkyl, alkenyl, alkynyl, heteroalkyl, benzyl and heterocyclyl moiety of R₇ and R₇ₐ is independently optionally substituted with one or more groups independently selected oxo, —OH, —CN, C₁-C₆ alkyl, C₁-C₆ alkoxy, —NO₂, —N(R₃)(R₃ₐ), halo, —SH and mono- to per-halogenated C₁-C₆ alkyl.

13. The compound according to claim 1, wherein Y is selected from the group consisting of aromatic polycycle, non-aromatic polycycle, mixed aryl and non-aryl polycycle, polyheteroaryl, non-aromatic polyheterocycle, mixed aryl and non-aryl polyheterocycle, each of which is optionally substituted.

14. The compound according to claim 1, wherein Y is selected from the group consisting of —(O)C—C₀-C₃alkyl-aryl, —C₀-C₃alkyl-aryl, —C₀-C₃alkyl-aryl-O—C₂-C₄alkyl-N(R₃)(R₃ₐ), —C₀-C₃alkyl-heteroaryl-O—C₂-C₄alkyl-N(R₃)(R₃ₐ), —C₀-C₃alkyl-aryl-C₀-C₃alkyl, C₀-C₃alkyl-heteroaryl-C₀-C₃alkyl and aryl-C₁-C₃alkyl-aryl, each of which is optionally substituted.

15. The compound according to claim 1, wherein Y is aryl-C₁-C₃alkyl-aryl, wherein C₁-C₃ alkyl is optionally substituted with C₀-C₃alkyl.

16. The compound according to claim 1, wherein L is a covalent bond and Z is selected from the group consisting of
—C₀-C₇ alkyl-N(R₃)—C(O)-heterocyclyl-C₀-C₆ alkyl-, wherein the C₁-C₇ alkyl is optionally substituted with —C₀-C₆ alkyl-C(O)OR₃ or —C₀-C₃alkyl-OR₃,
C₀-C₇ alkyl-O—C(O)-heterocyclyl-C₀-C₆ alkyl-, wherein the C₁-C₇ alkyl is optionally substituted with —C₀-C₃alkyl-C(O)OR₃ or —C₀-C₃alkyl-OR₃, and
—C₁-C₄alkyl-N(R₃)C(O)-heteorcyclyl-C₁-C₇alkyl, wherein the C₁-C₄alkyl is optionally substituted with C₀-C₃alkyl-C(O)OR₃ or C₀-C₃alkyl-OR₃.

17. The compound according to claim 1, wherein X is —S—, —SO—, —SO₂—, —O—, —NR₃—, —N(OH)—CH₂—, —CH(OH)—, or

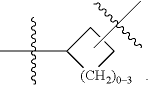

18. The compound according to claim 1, wherein R₁ and R₂ are independently —H, halo;
C₁-C₃ alkyl, C₃-C₆ cycloalkyl, aryl, heteroaryl, or aryl-C₁-C₃ alkyl-.

19. The compound according to claim 3, wherein R₁ and R₂ are independently —CH₃, —CH₂CH₃, phenyl, benzyl or benzofuran.

20. The compound according to claim 1, wherein R₁ and R₂ together with the carbon atom to which they are attached form a aryl or heteroaryl and 3- to 6-membered cycloalkyl or heterocyclyl group.

21. The compound according to claim 1, wherein R₃ and R₃ₐ are independently —H, C₁-C₆ alkyl, C₃-C₆ cycloalkyl, —C(O)CF₃, —C(O)H, C₁-C₄alkyl —C(O)OR₃; heterocyclyl; C₂-C₄ alkyl-OR₃, C₁-C₃alkylene; C₂-C₆alkenyl; C₂-C₆ hydroxyalkyl —C₁-C₆ alkylaryl, aryl; heteroaryl, C₀-C₆alkylheteroaryl; or C₁-C₃alkyl —C(O)NR₃-heteroaryl.

22. The compound according to claim 6, wherein R₃ and R₃ₐ are independently —C₁-C₆ alkylaryl, or aryl.

23. The compound according to claim 7, wherein R₃ and R₃ₐ are independently ethanol; tetrahydro-2H-pyran; phenyl or benzyl.

24. The compound according to claim 6, wherein R₃ and R₃ₐ are independently C₁-C₄ alkyl.

25. The compound according to claim 9, wherein R₃ and R₃ₐ are independently t-butyl or i-propyl.

26. The compound according to claim 1, wherein in a NR₃R₃ₐ group or a NR₄R₄ₐ group, optionally the R₃ together or the R₄ together with the nitrogen atom to which they are attached form a group selected from morpholinyl, piperazinyl,piperidinyl, pyrrolydinyl, and azetidinyl

27. The compound according to claim 1, wherein X-Q is —OH, —NH₂, —Cl, —F, —SH or —Br.

28. The compound according to claim 1, wherein X-Q is absent and R₁ and R₂ together with the carbon atom to which they attached form a 5- to 6-membered aromatic or heteroaromatic ring.

29. The compound according to claim 1, Q is selected from the group consisting of

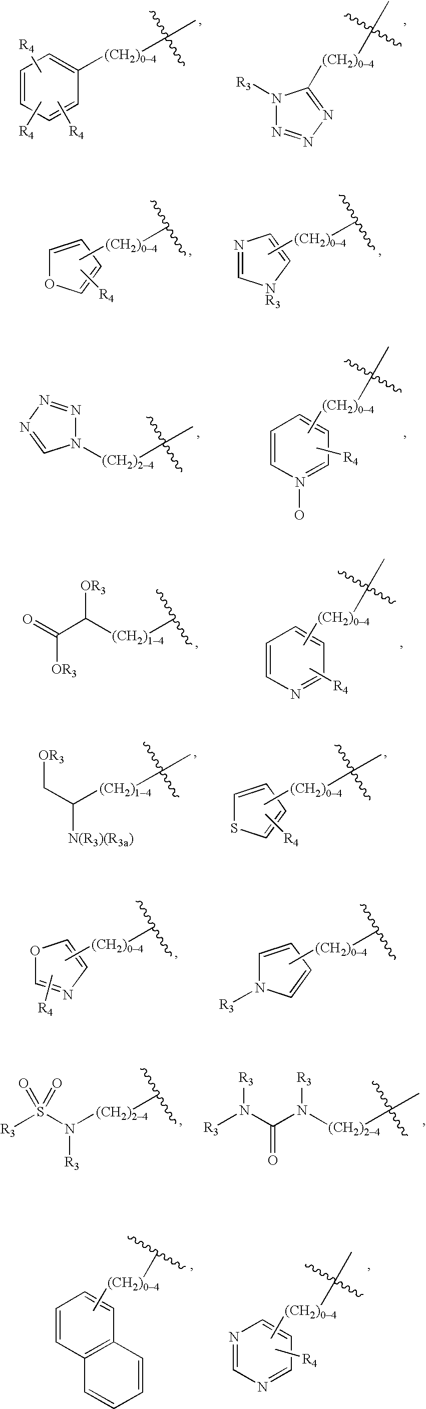

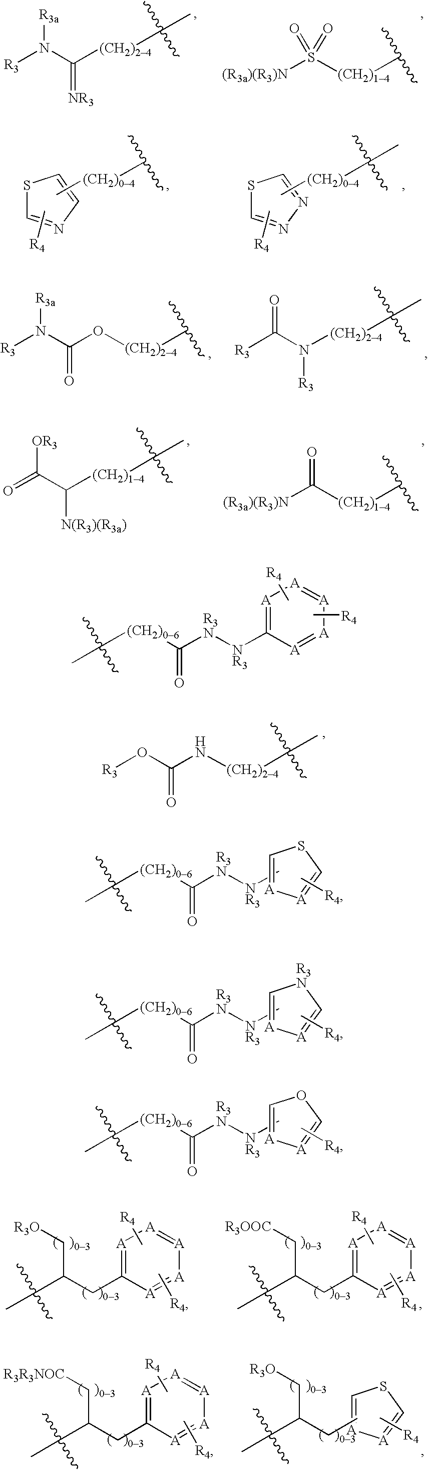

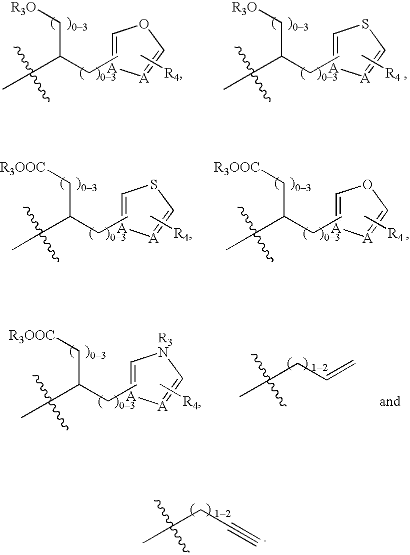

30. The compound according to claim 1, wherein R₄ is —H, —CH₃, —S(O)₂—N(R₃)R₃ₐ, —SO₃H, —O—C₂-C₄alkyl-heterocyclyl, —NR₃—C₂-C₄alkyl-heterocyclyl, —(CH₂)₀₋₄OR₃, —(CH₂)s ₄N(R₃)(R₃ₐ), —F, —Cl, —Br, —CF₃, —CN, —CH₂OH, —NO₂, —N(R₃)C(O)CH₂R₃, —N(R₃)SO₂CH₂R₃, —O(CH₂)₂AN (R₃)(R₃ₐ), —SR₃, —S(O)CH₂R₃, —SO₂CH₂R₃, —(CH₂)₀₋₄C(O)OR₃, —CH═CHC(O)OR₃, —CH═CHC(O)N(R₃)(R₃ₐ), —N(R₃)C(O)CF₃ or N(R₃)(CH₂)₂N(R₃)(R₃ₐ).

31. The compound according to claim 1, wherein Z is one of the following structures 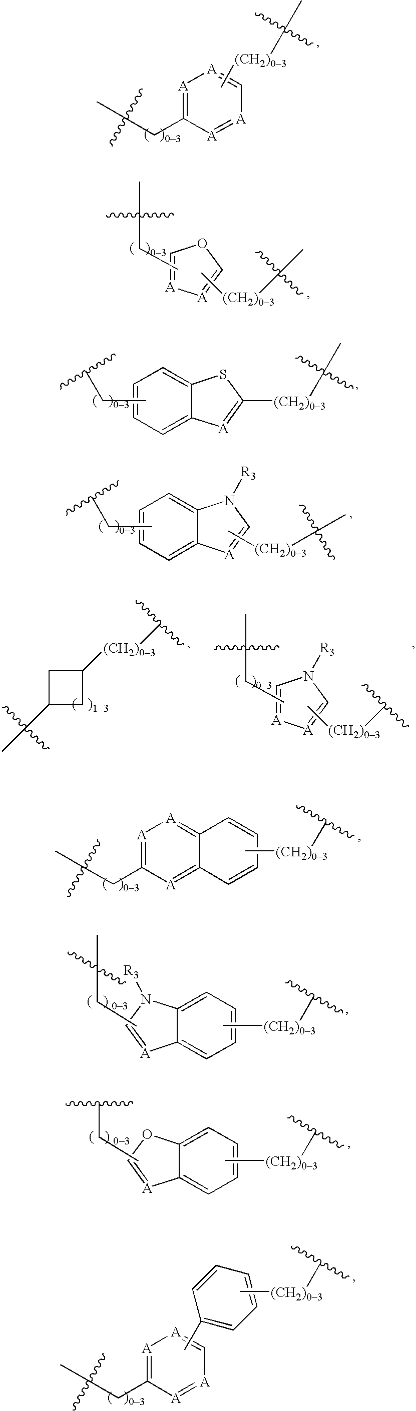 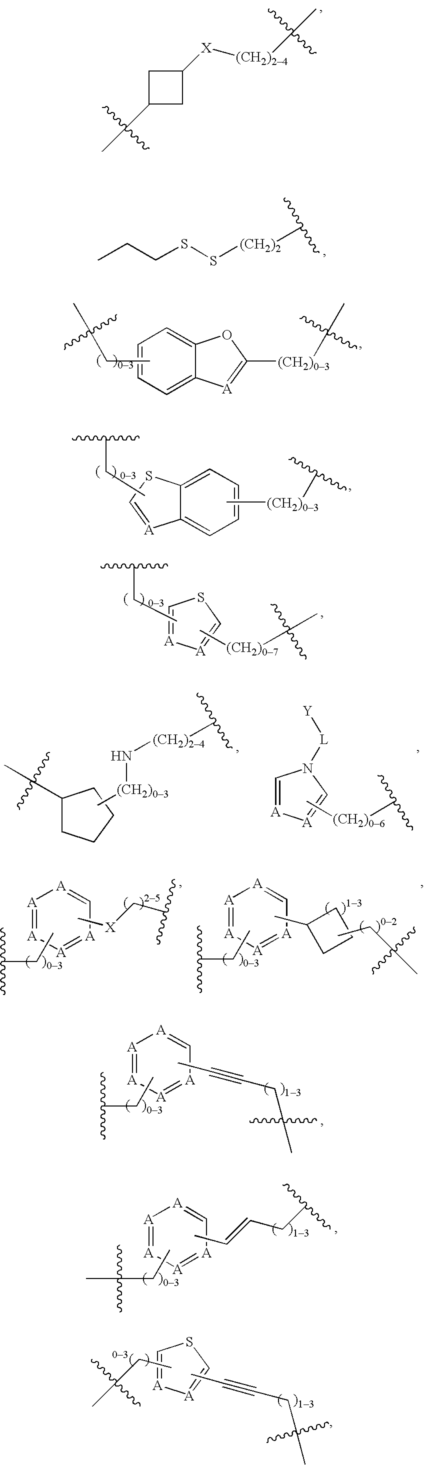 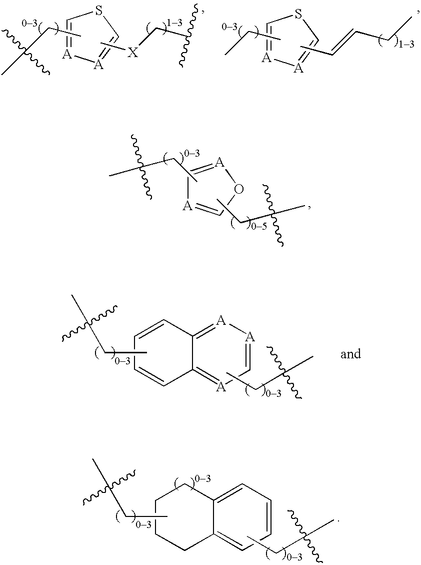
wherein A is —CH═ or —N═.

32. The compound according to claim 1, wherein L is selected from the group consisting of a covalent bond, —(CH₂)₀₋₃ N(R₃)C(O)—, —(CH₂)₀₋₃C(O)N(R₃)—, —(CH₂)_{0-3O}C(O)—, —(CH₂)₀₋₃C(O)O—, —C₀-C₆ alkyl-N(R₃)C(O)heterocyclyl-C₀-C₃alkyl, —C₀-C₆ alkyl-S(O)₂heterocyclyl-C₀-C₃alkyl,-, —(CH₂)₀₋₃C(O)—(CH₂)₀₋₃, —(CH₂)₀₋₃ SO₂N(R₃)—(CH₂)₀₋₃, —(CH₂)₀₋₃ NR₃S(O)₂—(CH₂)₀₋₃, —(CH₂)₀₋₃N(R₃)—(CH₂)₀₋₃, —(CH₂)₀₋₃N(R₇)—(CH₂)₀₋₃, —(CH₂)₀₋₃S—(CH₂)₀₋₃, —(CH₂)₀₋₃, —(CH₂)₀₋₃, —(CH₂)₀₋₃S(O)—(CH₂)₀₋₃, —(CH₂)₀₋₃S(O)₂—(CH₂)₀₋₃, —(CH₂)₀₋₃CH═CH—(CH₂)₂₋₃—, —(CH₂)₀₋₃N(R₃)C(O)N(R₃)—(CH₂)₀₋₃, —(CH₂)₀₋₃N(R₃)C(O)O—(CH₂)₀₋₃ and —(CH₂)₀₋₃OC(O)N(R₃)—(CH₂)₀₋₃, —(CH₂)₀₋₃ NR₃C(O)NR₃S(O)₂—(CH₂)₀₋₃, —(CH₂)₀₋₃ NR₃C(O)NR₃C(O)—(CH₂)₀₋₃, and —(CH₂)₀₋₃—C(O)—N(R₃)—C(O)N(R₃)—(CH₂)₀₋₃ and —(C₀-C₃alkyl)(R₃)N—S(O)₂—N(R₃)—C₂-C₄alkyl-O—C₀-C₃alkyl, -and R₃R₃ₐNS(O)₂NR₃—C₂-C₄alkyl-O—C₀-C₆ alkyl-, when Y is absent, and —R₃R₃ₐNS(O)₂NR₃—C₂-C₄alkyl, when Y is absent.

33. The compound according to claim 1, wherein L is selected from the group consisting of

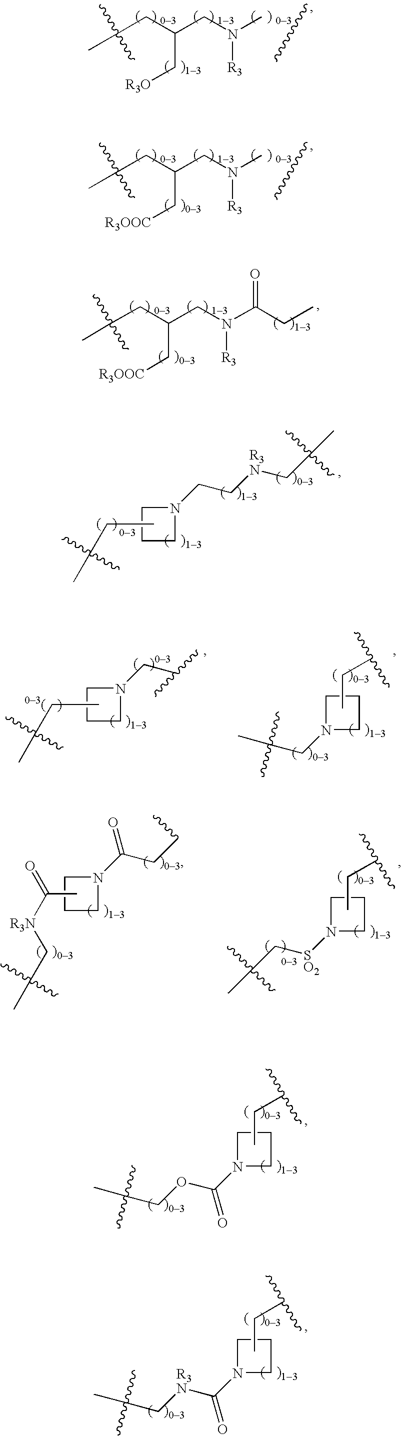

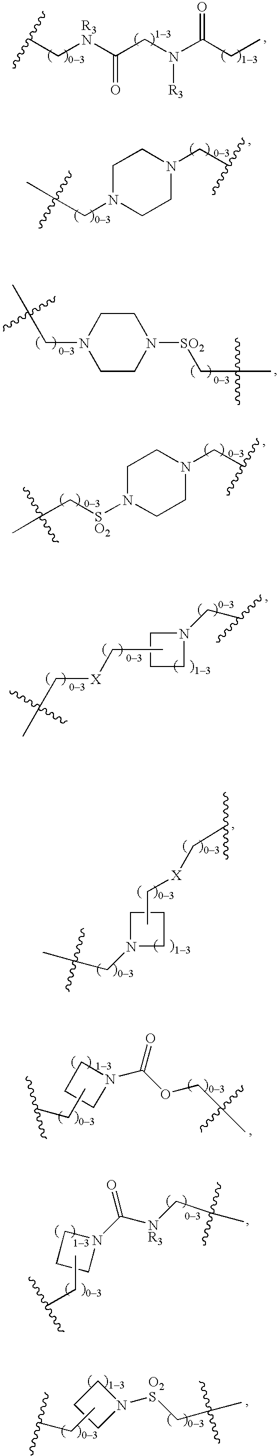

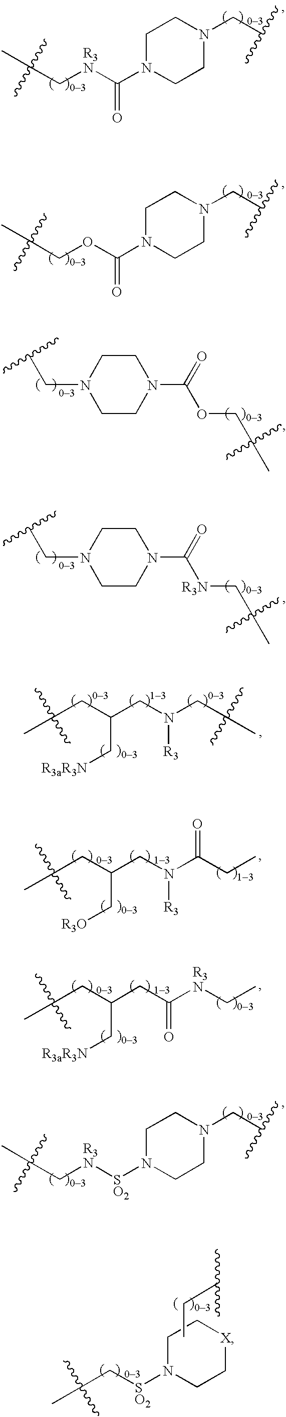

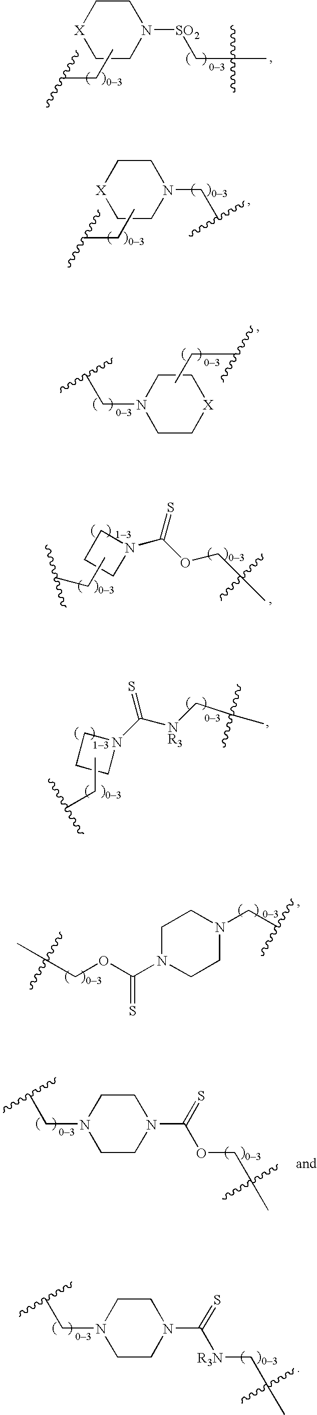

34. The compound according to claim 1, wherein Y is selected from the group consisting of 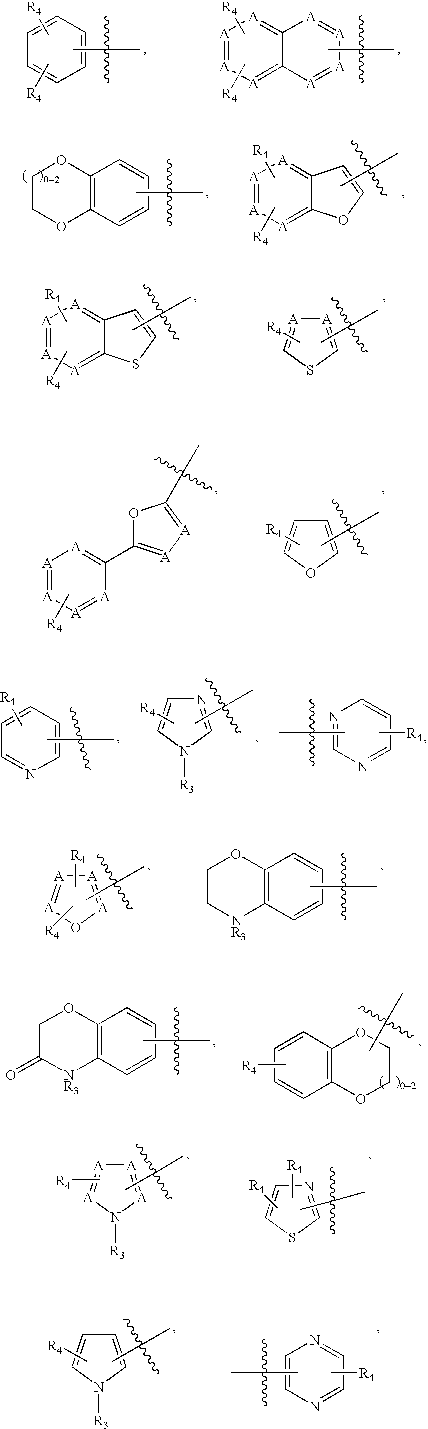 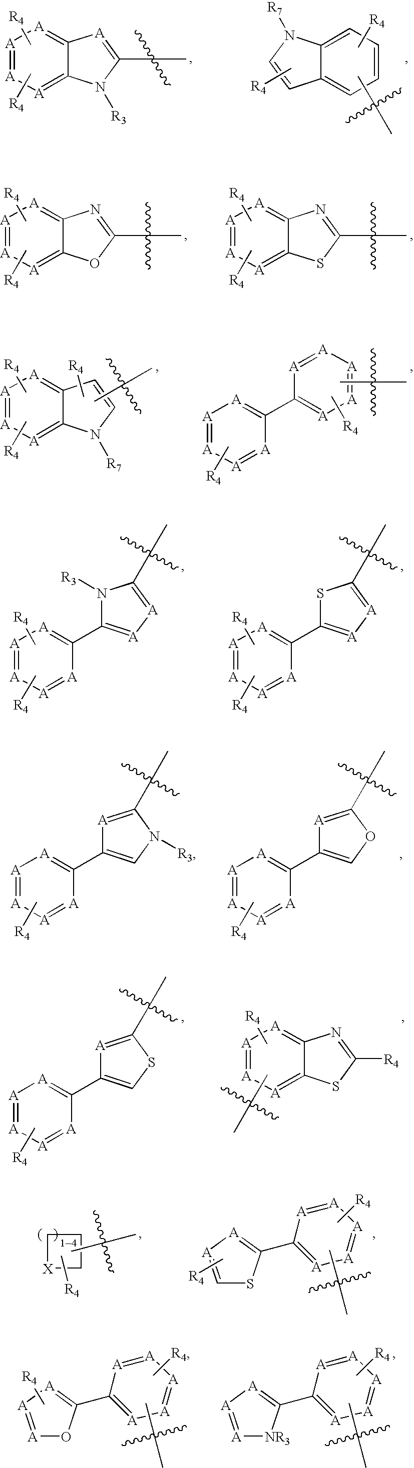 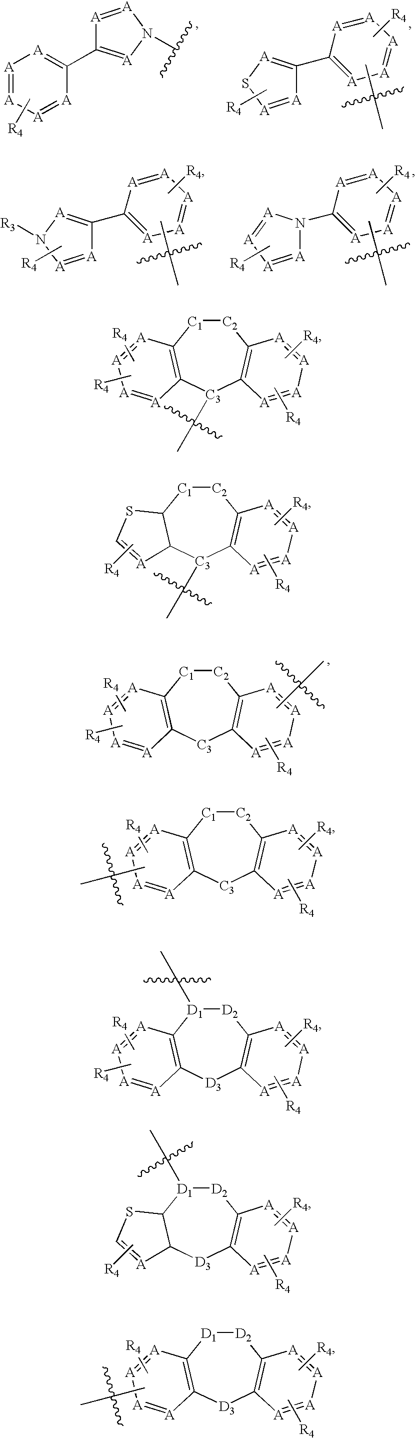 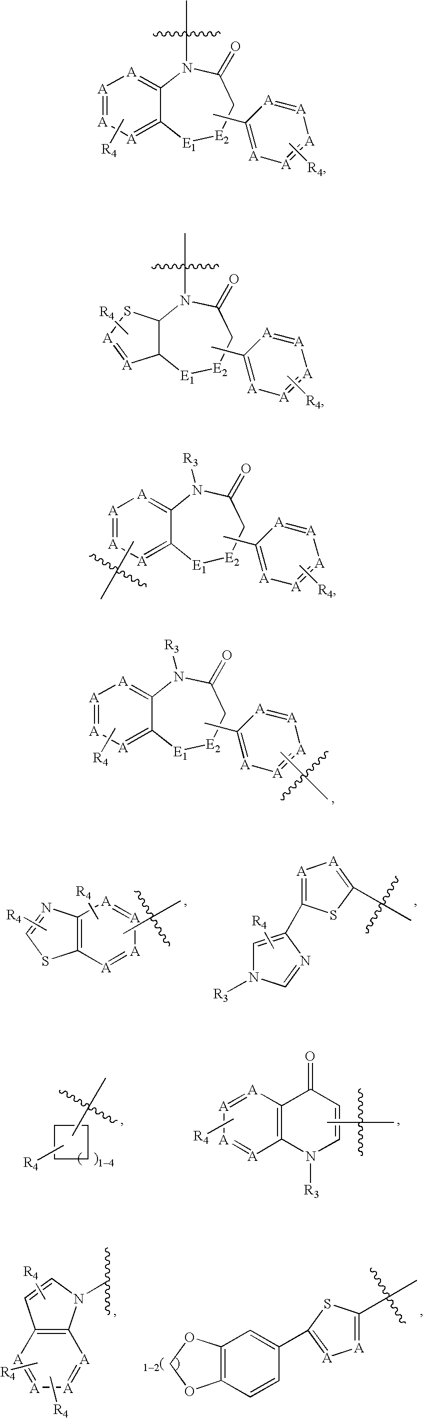 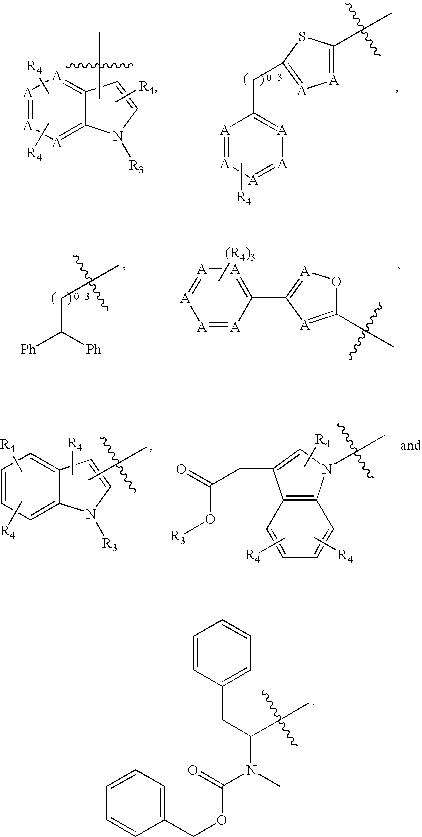
wherein A is —CH═ or —N═;
C₁ is selected from the group consisting of absent, a covalent bond, CH, CH₂, S, O, SO₂, C(O) and CR₃R₃;
C₂ is selected from the group consisting of absent, a covalent bond, CH, CH₂ and NR₃;
C₃ is selected from the group consisting of CH, N and NR₃;
D₁ is selected from the group consisting of N, CO and CH₂;
D₂ is selected from the group consisting of C, N and CH,
D₃ is selected from the group consisting of O, NR₃, SO and S;
E₁ is selected from the group consisting of S, C and N; and
E₂ is selected from the group consisting of CH, N and C(O).

35. The compound according to claim 1, wherein Y-L-Z- is one of the following structures 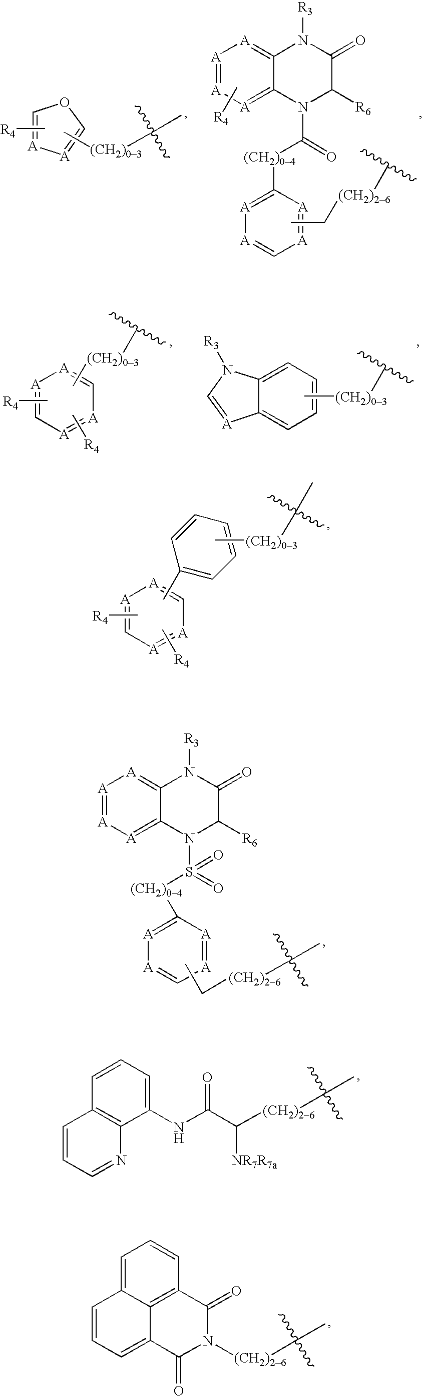 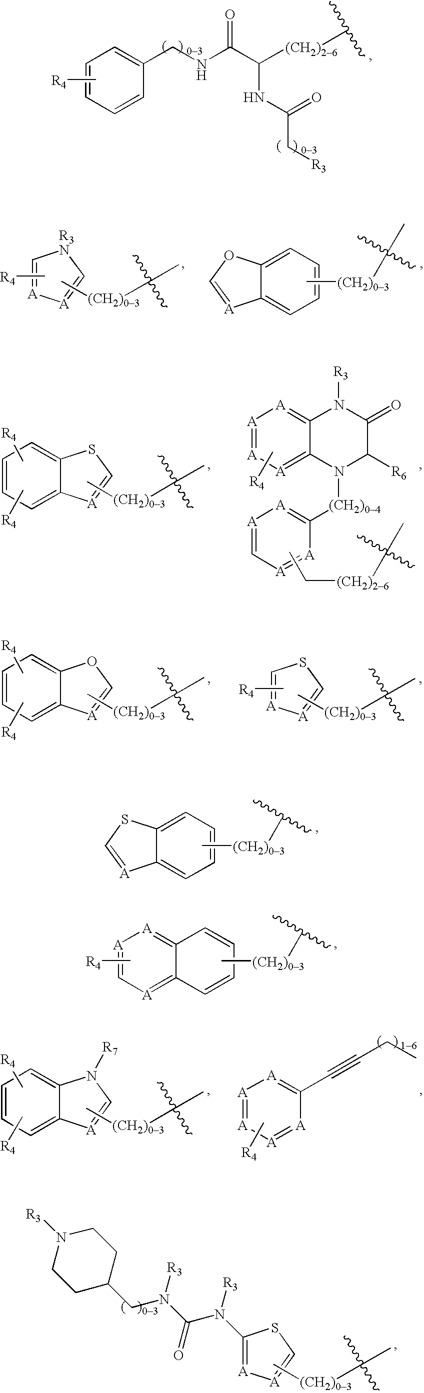 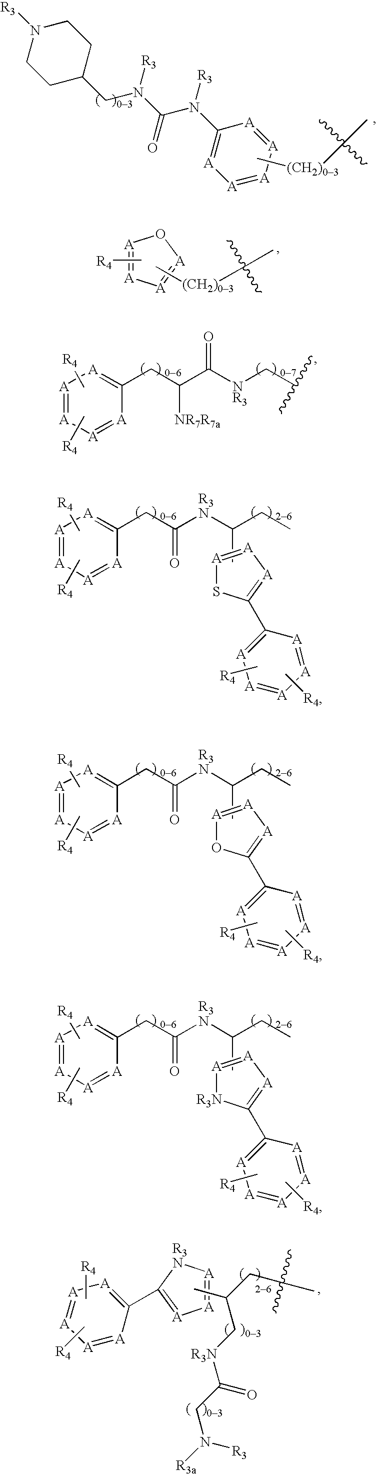 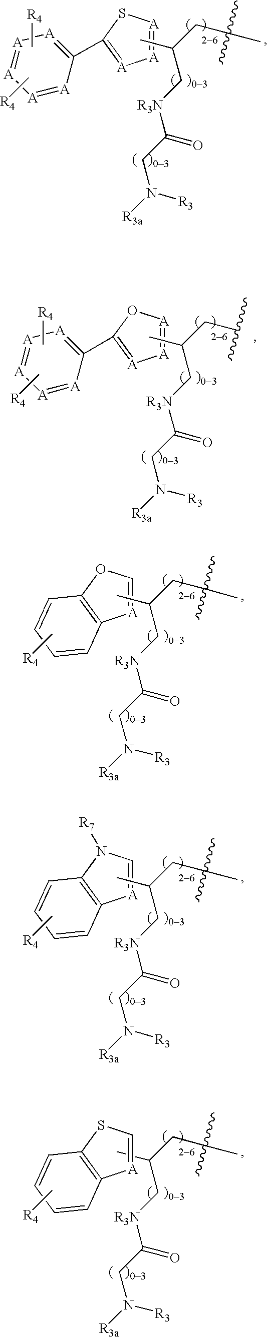 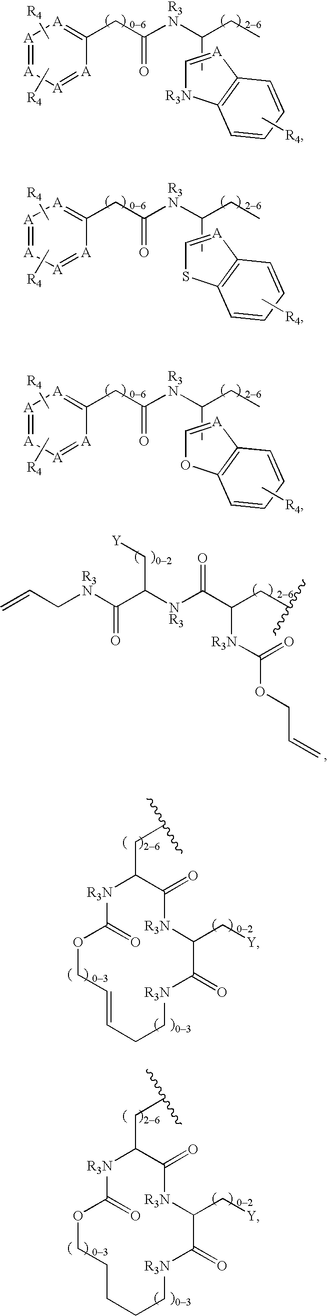 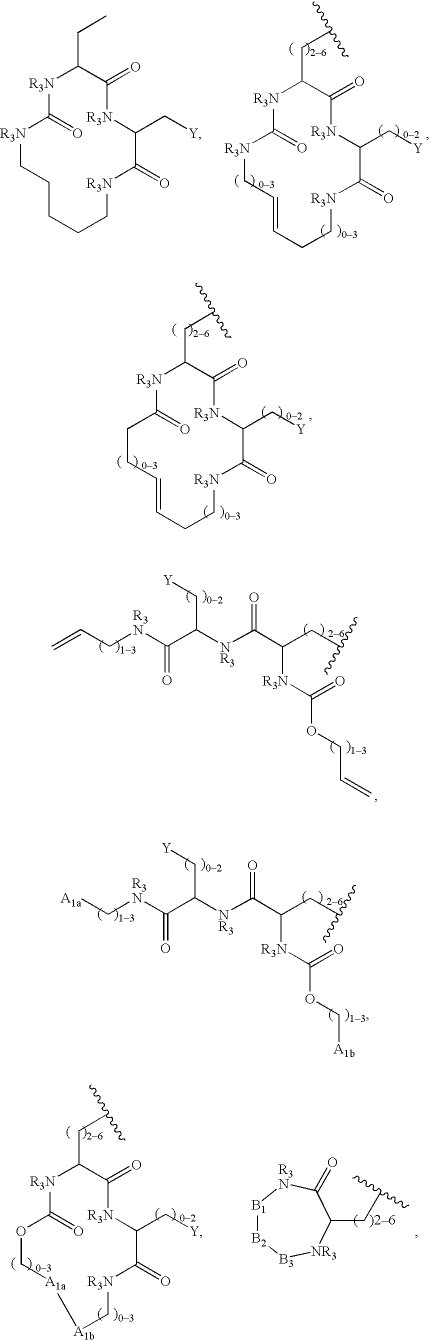 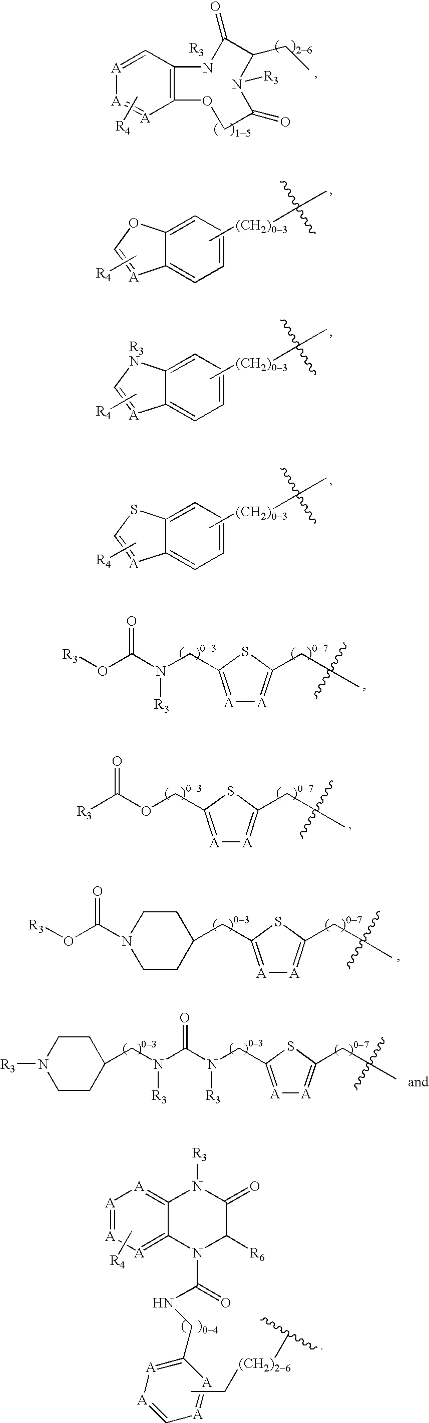
wherein A is —CH═ or —N═;
A₁ₐ and A_{1b} are independently selected from the group consisting of alkyl, alkenyl and protecting group; or
A₁ₐ and A_{1b} together —C₂-C₆alkylene, —C₂-C₆alkenylene or —C₂-C₆alkynylene linker, form an optionally substituted ring; and
B₁, B₂ and B₃ each independently a natural or synthetic amino acid.

36. The compound according to claim 29, wherein B₁, B₂ and B₃ are independently selected from the group consisting of D-Gly, L-Gly, D-Pro, L-Pro, D-Tyr, L-Tyr, D-Tyr(OR₃), L-Tyr(OR₃), D-Phe, L-Phe, D-PheR₄, L-PheR₄, D-Aib, L-Aib, D-Ala, L-Ala, D-ProR₃, L-ProR₃, D-Ile, L-Ile, D-Leu, L-Leu D-PheR₃, L-PheR₃, D-Pip and L-Pip.

37. The compound according to claim 1, wherein L is a covalent bond and Z- is selected from the group consisting of

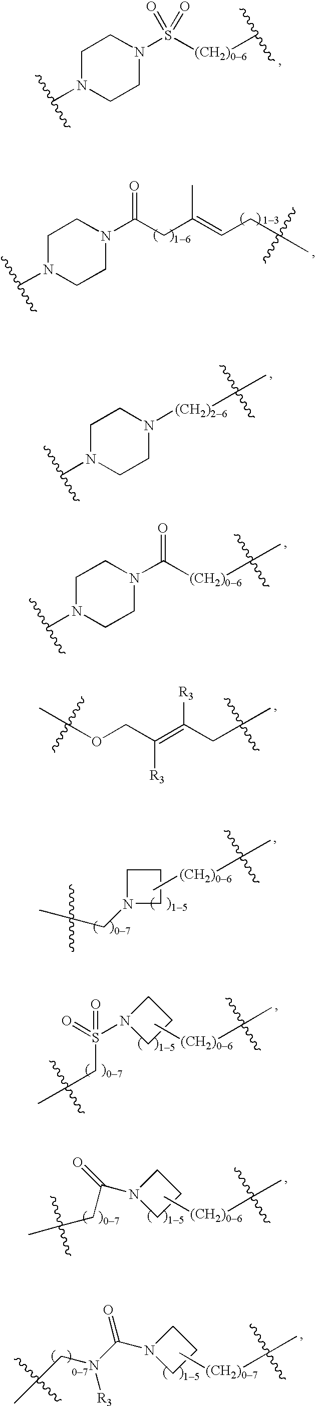

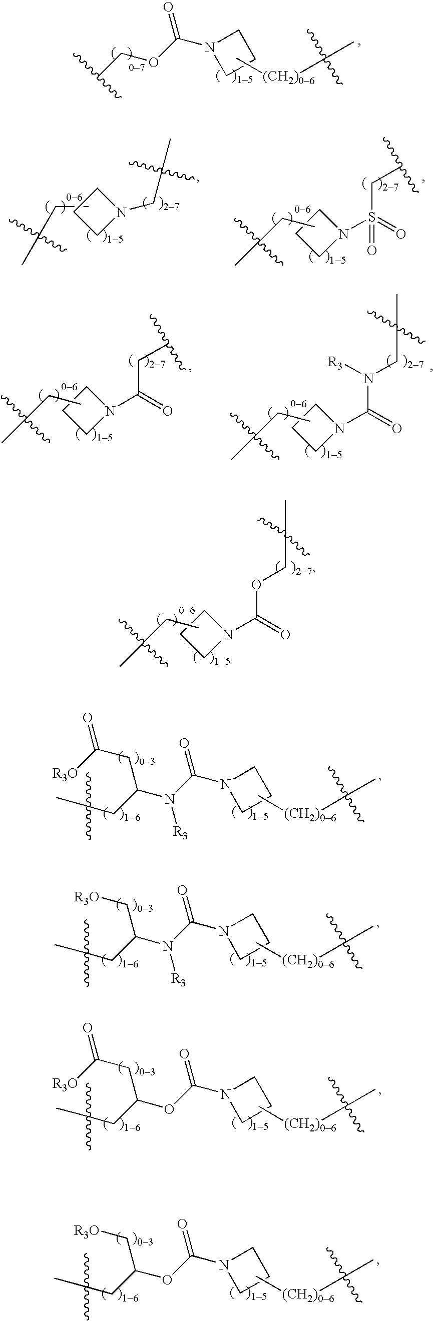

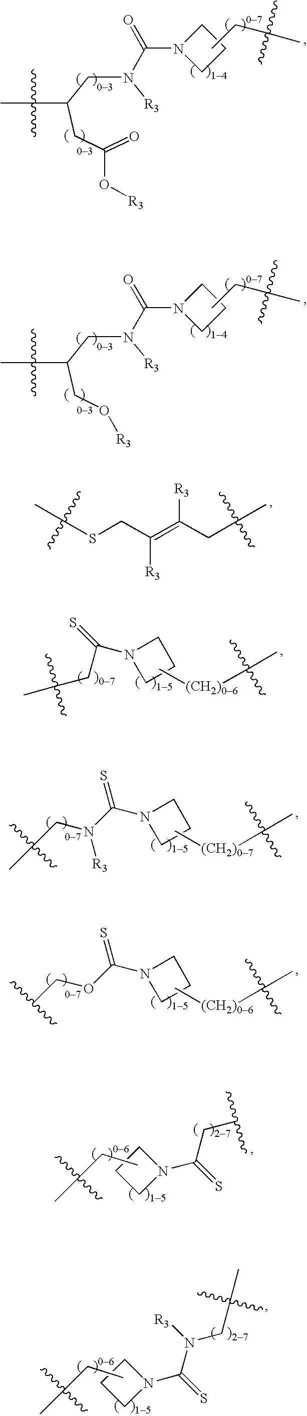

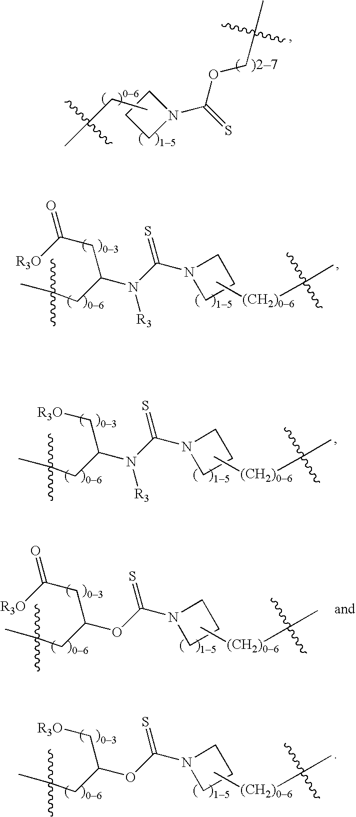

38. The compound according to claim 1, wherein R₆ is selected from the group consisting of

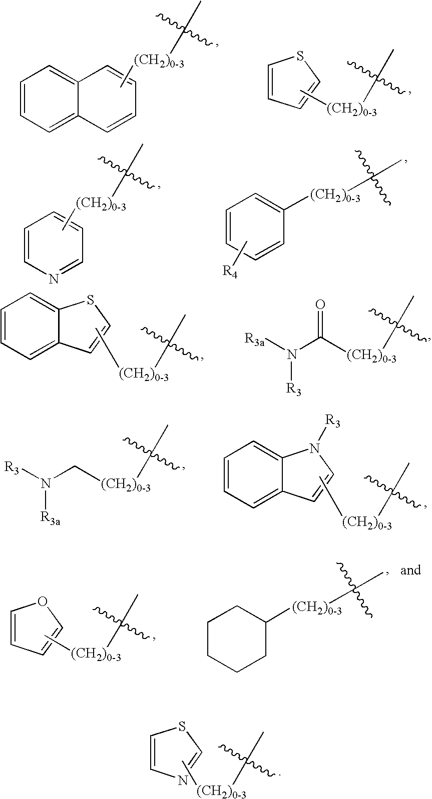

39. The compound according to claim 1, wherein R₇ is selected from the group consisting of —H, optionally substituted C₁-C₆ alkyl, —(CH₂)₂₋₄OR₃, —(CH₂)₂₋₄N(R₃)(R₃ₐ), —C(O)Ot-butyl, —C(O)O-benzyl, —(CH₂)₂-morpholinyl or —(CH₂)₂-piperazynnyl.

40. The compound according to claim 1, wherein then Q is —C(O)—OR₃ and X is —N(R₃)—, —C(H)₂— —CH(OH)—.

41. The compound according to claim 1, wherein Q is —C(O)R₃ and X is —SO—, —O— or —N(R₃)—.

42. The compound according to claim 1, wherein X is —CH₂— and 0 is selected from the group consisting of —(CH₂)₀₋₃—X—(CH₂)₁₋₃—C(O)OR₃, —(CH₂)₀₋₃—X—(CH₂)₂₋₃—OR₃ and —(CH₂)₀₋₃—X—(CH₂)₂₋₃ON(R₃)(R₃ₐ).

43. The compound according to claim 1, wherein
W is nitrogen;
X is a covalent bond or —CH₂—;
R₁ and R₂ are —H
R₃ is —H, —OH, —C(O)—NH-aryl, —C(O)—NH₂, —C(NH₂)—C(O)—OH, NH₂, —C(NH₂)—C(O)—O—C₁-C₆ alkyl, —C(O)—OH, —C(O)—O—C₁-C₆ alkyl, aryl, heteroaryl, wherein each of the aryl and heteroaryl is optionally substituted with one or more groups selected from —OH, —CN, C₁-C₆ alkyl, —N(R₄)(R₄ₐ), halo;
Q is —C₁-C₆ alkyl-N(R₃)—C(O)—C₁-C₆ alkyl-B—(CH₂)ₙ—R₃;
B is —O—, —S(O)—, or —S—;
n is 0 or an interger from 1 to 3;
R₄ and R₄ₐ are —H; and
Y-L-Z- is aryl-C₀-C₆ alkyl-aryl-C₀-C₆ alkyl-, aryl-C₀-C₆ alkyl-aryl-C₀-C₆ alkenyl-, aryl-C₀-C₆ alkyl-aryl-C₀-C₆ alkynyl-, aryl-C₀-C₆ alkenyl-aryl-C₀-C₆ alkyl-, aryl-C₀-C₆ alkenyl-aryl-C₀-C₆ alkenyl-, aryl-C₀-C₆ alkenyl-aryl-C₀-C₆ alkynyl-, aryl-C₀-C₆ alkynyl-aryl-C₀-C₈ alkyl-, aryl-C₀-C₆ alkynyl-aryl-C₀-C₆ alkenyl-, aryl-C₀-C₆ alkynyl-aryl-C₀-C₆ alkynyl-.

44. The compound according to claim 43 of the formula 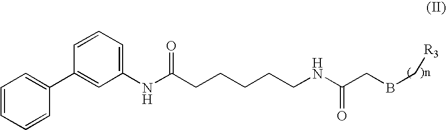
(II)
or a pharmaceutically acceptable salt thereof, wherein B, n and R₃ are any one of the following combination:

| n | B | R₃ | |
|---|---|---|---|
| 1 | O | 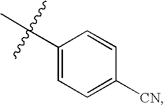 | |
| 1 | O | 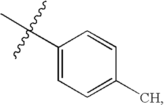 | |
| 1 | O | 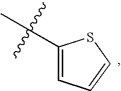 | |
| 1 | O | 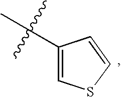 | |
| 1 | O | 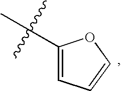 | |
| 1 | O | 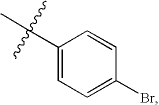 | |
| 1 | O | 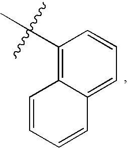 | |
| 1 | S O | 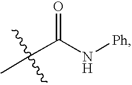 | |
| 2 | S | 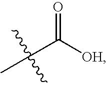 | |
| 2 | S O | 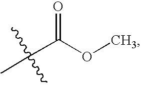 | |
| 3 | S | —OH, | |
| 2 | S | —OH, | |
| 1 | S | 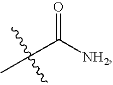 | |
| 1 | S | 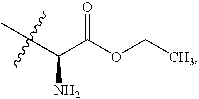 | |
| 1 | S | 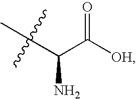 | |
| 2 | S | —NH2 | |
| 0 | S | 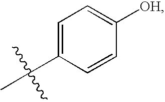 | |
| 1 | S O | 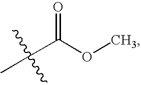 | |
| 2 | S | 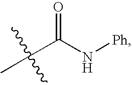 | |
| 2 | S O | 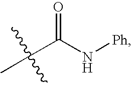 | |
| 0 | S | 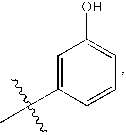 | |
| 0 | S | 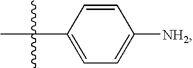 | |
| 0 | S | 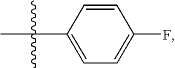 | |
| 0 | S | 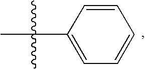 | |
| 0 | S | 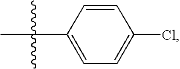 | |
| 0 | S | 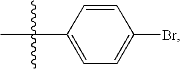 | |
| 0 | S | 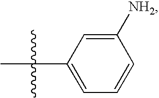 | |
| 0 | S | 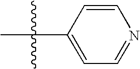 | or |
| 0 | S | 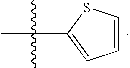 | |

45. The compound according to claim 1, wherein
W is nitrogen;
X is a covalent bond or —CH₂—;
R₁ and R₂ are —H;
R₃ is H, aryl or heteroaryl, wherein each of the aryl and heteroaryl is optionally substituted with one or more groups selected from —CN, —S(O)—C₁-C₆ alkyl, C₁-C₆ alkyl, or halo;
Q is —C₁-C₆ alkyl-N(R₃)—C(O)—C₁-C₆ alkyl-B—C₁-C₆ alkyl-R₃;
B is —S—, —S(O)— or —O—;
and
Y-L-Z- is aryl-C₀-C₆ alkyl-aryl-C₀-C₆ alkyl-.

46. The compound according to claim 45 of the formula 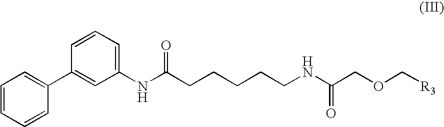
or a pharmaceutically acceptable salt thereof, wherein R₃ is selected from the group consisting of

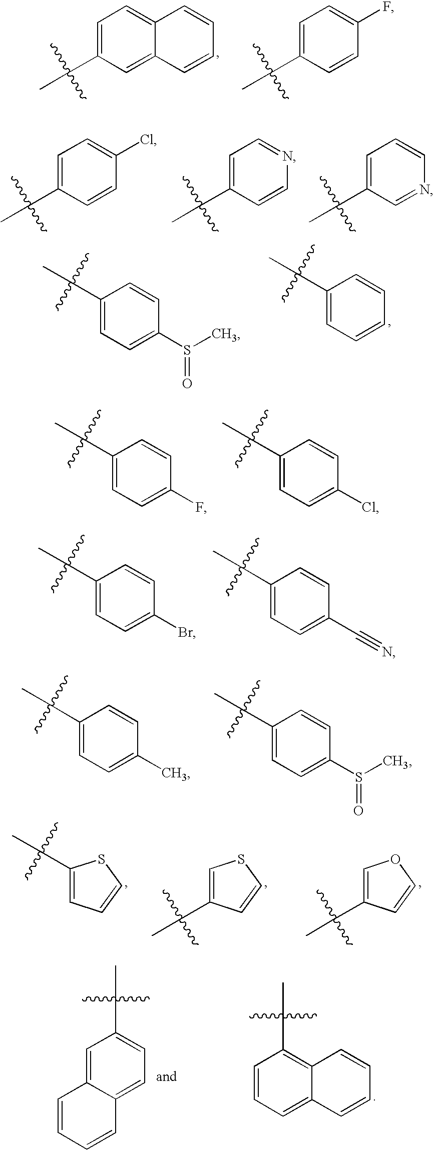

47. The compound according to claim 1, wherein
W is nitrogen;
X is a covalent bond or —CH₂—;
R₁ and R₂ are —H;
R₃ is —H, —C₁-C₆ hydroxyalkyl-C(O)—OH, —C₁-C₆ alkyl-S—C₁-C₆ alkyl-C(NH₂)—C(O)—OR₄, —C₁-C₆ alkyl-S(O)—C₁-C₆ alkylaryl, —C₁-C₆ alkyl-S—C₀-C₆ alkylaryl, —C₁-C₆ alkyl-S—C₀-C₆ alkylheteroaryl, —C₁-C₆ alkyl-S—C₁-C₆ alkyl-OH, —C₁-C₆ alkyl-S—C₁-C₆ alkyl-C(O)—OH, —C₁-C₆ alkyl-S—C₁-C₆ hydroxyalkyl-C(O)—O—C₁-C₆ alkyl, —C₁-C₆ alkyl-S—C₁-C₆ alkyl-C(O)—O—C₁-C₆ alkyl, —C₁-C₆ alkyl-S(O)—C₁-C₆ alkyl-C(O)—OR₄, —C₁-C₆ alkyl-S—C₁-C₆ alkyl-C(O)—N(R₄)(R₄ₐ), —C₁-C₆ alkyl-S(O)—C₁-C₆ alkyl-C(O)—N(R₄)-aryl, —C₁-C₆ alkyl-S—C₁-C₆ alkyl-N(R₄)(R₄ₐ), and —C₁-C₆-alkylheteroaryl, wherein each of the aryl and heteroaryl is optionally substituted with one or more groups selected from oxo, —OH, —N(R₄)(R₄ₐ), halo, —S—C₁-C₆ alkyl, or —S(O)—C₁-C₆ alkyl;
Q is —C₁-C₆ alkyl-N(R₃)—C(O)—R₃;
R₄ and R₄ₐ are independently —H, C₁-C₆ alkyl, or aryl; and
Y-L-Z- is aryl-C₀-C₆ alkyl-aryl-C₀-C₆ alkyl-.

48. The compound according to claim 47 of the formula 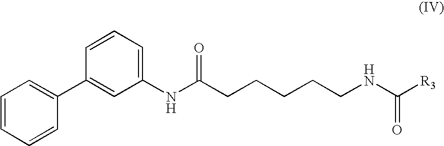
or a pharmaceutically acceptable salt thereof, wherein R₃ is selected from the group consisting of

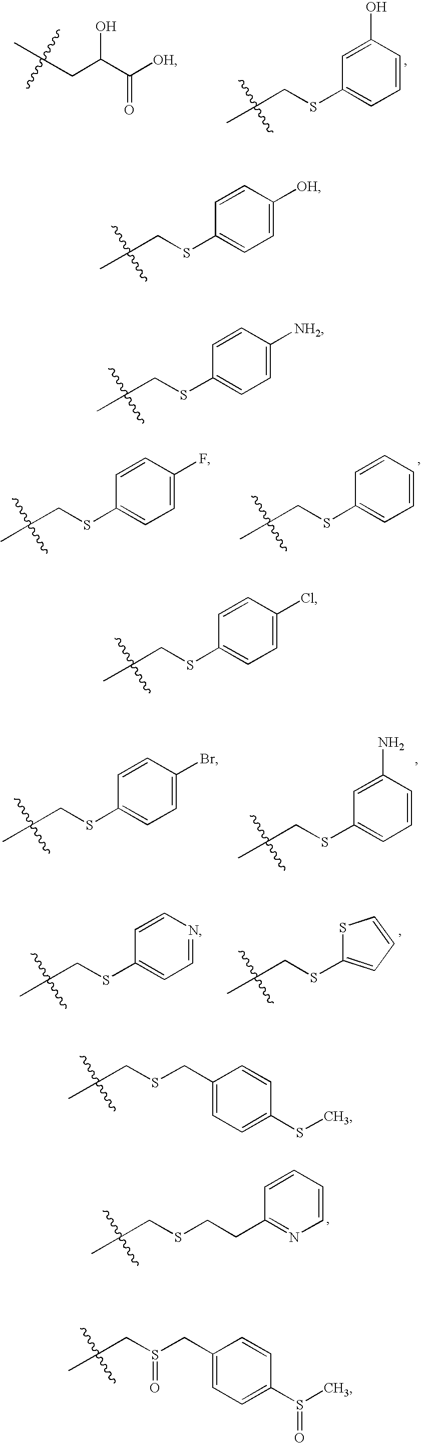

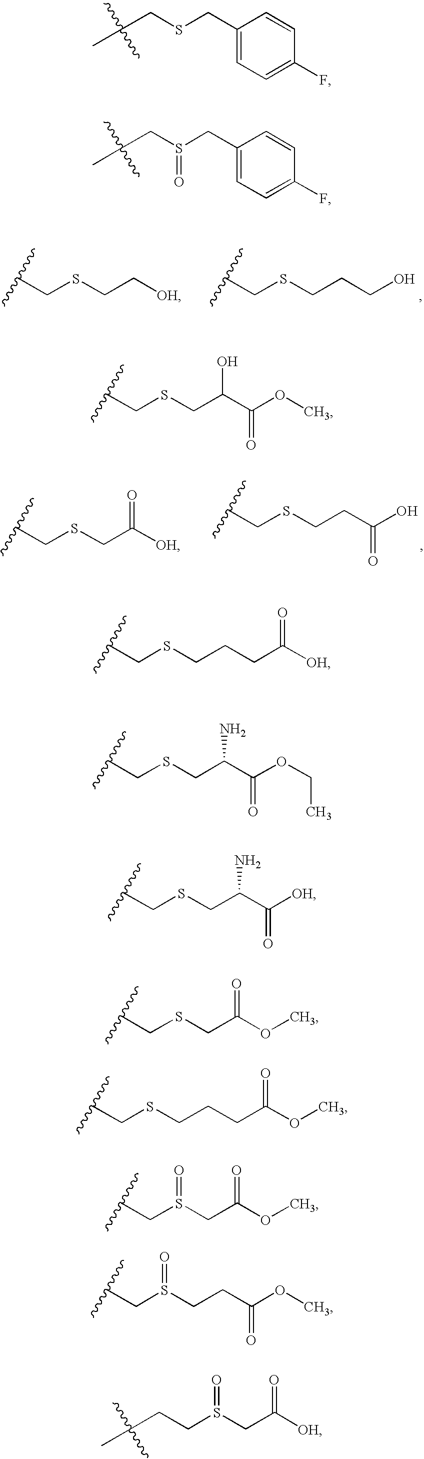

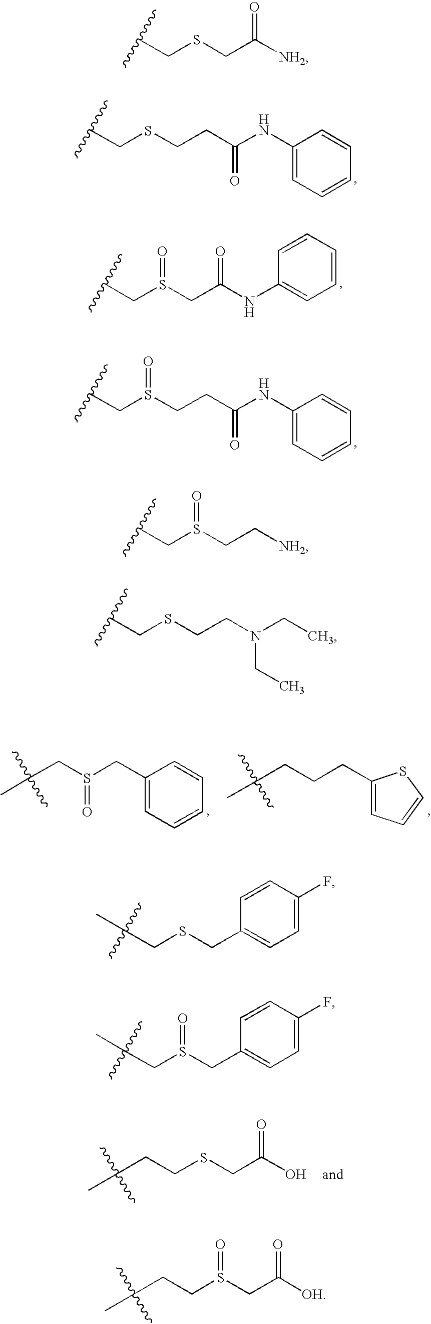

49. The compound according to claim 1, wherein
W is nitrogen;
X is —S—;
R₁ and R₂ are —H;
R₃ is —H;
Q is —C₁-C₆-alkyl-C(O)—R₃; and
Y-L-Z- is heteroaryl-C₀-C₆ alkyl- or heteroaryl-C₀-C₆ alkyl-heteroaryl-C₀-C₇ alkyl-aryl-C₀-C₆-alkyl, wherein each of the aryl and heteroaryl is optionally substituted with one or more groups selected from oxo or halo.

50. The compound according to claim 49 that is one of the following structures:

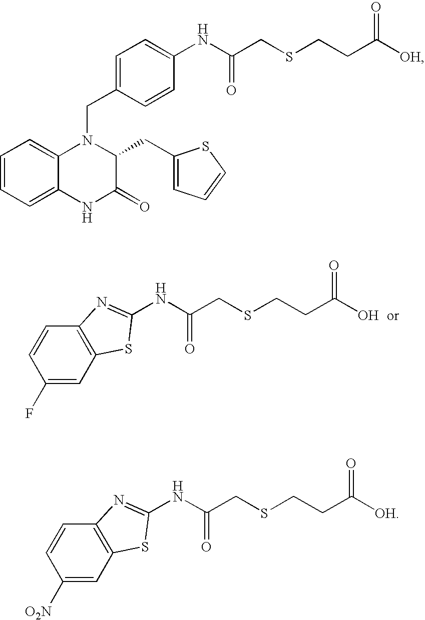

51. The compound according to claim 45 of the formula 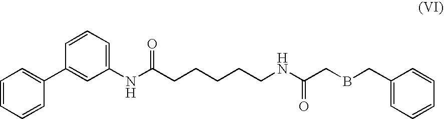
wherein B is —S— or —S(O)—.

52. The compound according to claim 1, wherein
W is nitrogen;
X is —S—;
R₁ and R₂ are —H;
R₃ is —H or C₁-C₆ alky;
R₄ is C₁-C₆ alkyl-OR₃;
Q is —C₁-C₆-alkyl-C(O)—OR₃, —C₁-C₆-alkyl-N(R₃)(R₃ₐ), C₁-C₆ alkyl substituted with —OH; and
Y-L-Z- is heteroaryl-C₁-C₆ alkyl-N(R₄)—C₁-C₆-alkyl-aryl-C₀-C₆ alkyl- or heteroaryl-C₀-C₆ alkyl-heteroaryl-C₀-C₇ alkyl-aryl-C₀-C₆-alkyl, wherein each of the aryl and heteroaryl is optionally substituted with one or more groups selected from oxo.

53. The compound according to claim 52 that is one of the following structures:

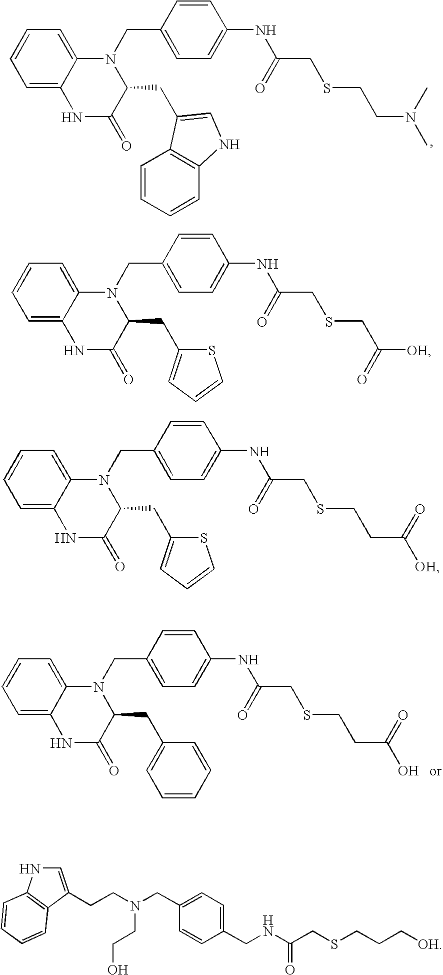

54. The compound according to claim 1, wherein
W is nitrogen;
X is a covalent bond or —CH₂—;
R₁ and R₂ are independently —H.—N(H)—C(O)—O—C₁-C₆ alkyl or —N(H)—C(O)—O-benzyl;
R₃ is —H or —C₁-C₆ alkyl-O—C₀-C₆ alkylaryl;
Q is —C₁-C₆ alkyl-N(R₃)—C(O)—R₃; and
Y-L-Z- is heteroaryl-C₀-C₆ alkyl-.

55. The compound according to claim 54 that is one of the following structures:

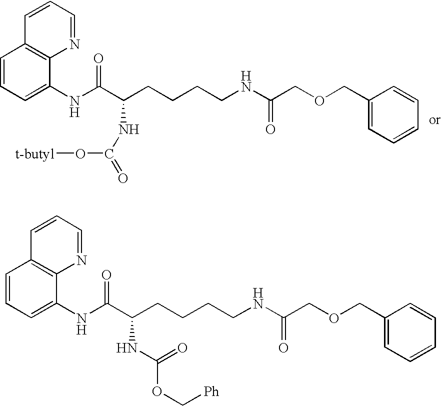

56. The compound according to claim 1, wherein
W is nitrogen;
X is —S—;
R₁ and R₂ are —H;
R₃ is —H;
Q is —C₁-C₆-alkyl-C(O)—OR₃; and
Y-L-Z- is heteroaryl-C₀-C₆ alkyl-, wherein the heteroaryl is optionally substituted with one or more groups selected from C₁-C₆ alkoxy, halo or —NO₂.

57. The compound according to claim 56 that is one of the following structures:

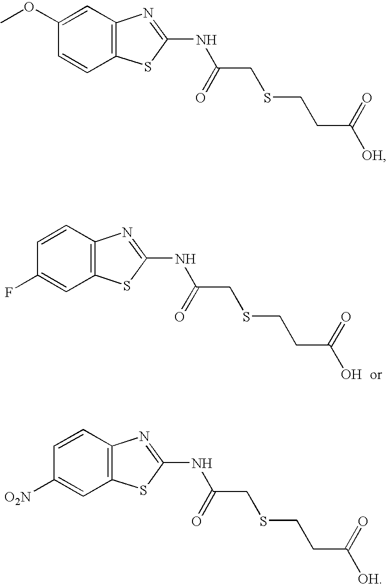

58. The compound according to claim 1, wherein
W is nitrogen;
X is selected from the group consisting of S, O, SO and SO₂;
R₁ and R₂ are H or halogen;
Q is selected from the group consisting of aryl-NH₂, C₁-C₆alkyl-aryl, C₁-C₆alkyl-heteroaryl, C₁-C₆alkyl-CN, wherein the alkyl, aryl and heteroaryl are each independently optionally substituted;
Z is selected from the group consisting of —C₁-C₆alkyl-, —C₁-C₈heteroalkyl-, —C₀-C₆alkyl-aryl-C₀-C₃alkyl-X—C₀-C₃alkyl-, —C₀-C₆alkyl-heteroaryl-C₀-C₃alkyl-X—C₀-C₃alkyl-, —C₀-C₆alkyl-heteroaryl-C₂-C₆heteroalkyl-, —C₀-C₃alkyl-X—C₀-C₃alkyl-, —C₀-C₆alkyl-aryl-C₃-C₆alkynyl- and —C₀-C₆alkyl-aryl-C₃-C₆alkenyl-, wherein the alkyl, heteroalkyl, aryl, heteroaryl and alkenyl are each independently optionally substituted;
L is selected from the group consisting of —C₀-C₆alkyl-S(O)₂—N(R₃)—C₀-C₆alkyl-, —C₀-C₆alkyl-O—C(O)—N(R₃)—C₀-C₃alkyl-, —C₀-C₆alkyl-N(R₃)—S(O)₂—C₀-C₃alkyl-, -heterocyclyl-C(O)—C₀-C₃alkyl-, —C₀-C₆alkyl-N(R₃)—C(O)—C₀-C₃alkyl-, —C₀-C₆alkyl-C(O)—N(R₃)—C₀-C₃alkyl-, covalent bond, —C₀-C₆alkyl-N(R₃)—C₀-C₃alkyl-, —C₀-C₆alkyl-, —S(O)₂—N(R₃)—C₀-C₃alkyl-aryl-C₀-C₃alkyl-C(O)—N(R₃)—C₁-C₃alkyl-, —C₀-C₆alkyl-S(O)₂—C₀-C₃alkyl-, —C₀-C₃alkyl-S(O)₂—N(R₃)—C₀-C₃alkyl-heterocyclyl-C₀-C₃alkyl-, —C₀-C₃alkyl-O—C(O)—N(R₃)—C₀-C₃alkyl-heterocyclyl-C₀-C₃alkyl-, —C₀-C₃alkyl-C(O)—N(R₃)—C₀-C₃alkyl-heterocyclyl-C₀-C₃alkyl-, —C₀-C₃alkyl-N(R₃)—C(O)—O-heterocyclyl-C₀-C₃alkyl and —C₀-C₆alkyl-C(O)—N(R₃)—C(O)—C₀-C₃alkyl-, wherein the alkyl, heterocyclyl and aryl are each independently optionally substituted; and
Y is selected from the group consisting of aryl-aryl, aryl, heterocyclyl-aryl, heteroaryl, heteroaryl-aryl, heterocyclyl, alkylaryl, alkylheterocyclyl, aryl-alkylheterocyclyl, heterocyclyl-alkyl-aryl, alkyl, heteroaryl-heteroaryl and heterocyclyl-heteroaryl, wherein each said Y is independently optionally substituted.

59. The compound according to claim 58, wherein Q is C₁-C₆alkyl-heteroaryl, wherein said C₁-C₆alkyl is optionally substituted with —CH₂—C(O)—O—C₁-C₆alkyl.

60. The compound according to claim 1, wherein
W is nitrogen;
X is —O— or —S—;
R₁, R₂ are H;
Q is selected from the group consisting of C₀-C₆alkyl-aryl and heteroaryl, wherein said alkyl, aryl and heteroaryl are independently optionally substituted;
Y-L-Z is selected from the group consisting of aryl-C₀-C₆alkyl-C(O)—N(R₃)—C₁-C₇alkyl-, (R₃)(R₃ₐ)N—C₀-C₆alkyl-C(O)—N(R₃)—C₁-C₇alkyl-, aryl-C₀-C₆alkyl-O—C(O)—N(R₃)—C₁-C₇alkyl-, C₀-C₃alkyl-heteroaryl-C₀-C₃alkyl-N(R₃)—C(O)—C₁-C₇alkyl-, C₁-C₇alkyl-N(R₃)—C(O)—C₀-C₆alkyl-, heteroaryl-C₀-C₆alkyl-C(O)—N(R₃)—C₁-C₇alkyl-, wherein each of said aryl, alkyl and heteroaryl are independently optionally substituted.

61. The compound according to claim 60, wherein said C₁-C₇alkyl is optionally substituted with a substituent selected from the group consisting of heteroaryl-aryl, —C(O)—N(R₃)-heteroaryl, —N(R₃)—C(O)—O-alkenyl, heteroaryl and —N(R₃)—C(O)—O—C₀-C₃alkyl-aryl, and said C₀-C₃alkyl is optionally substituted with —C(O)—N(R₃)alkenyl.

62. The compound according to claim 1, wherein
W is nitrogen;
X is —O— or —S—;
R₁ and R₂ are H;
Q is alkyl-aryl; and
Y-L-Z is selected from the group consisting of aryl-C₀-C₃alkyl-N(R₃)—C(O)—C₁-C₇alkyl-, A₂ₐ-aryl-C₀-C₃alkyl-N(R₃)—C(O)—C₁-C₇alkyl- and heteroaryl-C₀-C₃alkyl-N(R₃)—C(O)—C₁-C₇alkyl-, wherein said aryl and heteroaryl are each independently optionally substituted, and wherein said C₀-C₃alkyl is optionally substituted with —C(O)—N(R₃)—C₁-C₆alkyl-A₁ₐ or —C(O)—N(R₃)—CO—C₆alkyl-C(O)-A₂ₐ; and
said C₁-C₇alkyl is optionally substituted with a substituent selected from the group consisting of —N(R₃)—C(O)—O—C₁-C₃alkyl-A_{1b}, —N(R₃)—C(O)—C₁-C₇alkyl-O-A_{2b}, —N(R₃)—C(O)-heteorcyclyl-A_{2b} and —N(R₃)—C(O)—C₂-C₇alkenyl-O-A_{2b}, wherein
A₁ₐ and A_{1b} optionally together via a C₂-C₆alkylene, C₂-C₆alkenylene or C₂-C₆alkynylene linker, form an optionally substituted ring system; and
A₂ₐ and A_{2b} together are a covalent bond and are attached to form a ring, or
Y-L-Z is B₂—B₁—N(R₃)—C(O)—C₁-C₇ alkyl-, wherein the C₁-C₇ alkyl is optionally substituted with —NR₃—B₃ and the amine of B₃ is conected with the acid of B₂ to form a peptide bond; wherein
B₁, B₂ and B₃ are each independently a natural or synthetic amino acid.

63. The compound according to claim 1, wherein
W is nitrogen;
X is —O—;
R₁ and R₂ are H;
Q is optionally substituted alkyl-aryl;
Z is optionally substituted C₁-C₈alkyl;
L is selected from the group consisting of —C₀-C₃alkyl-N(R₃)—C(O)-heterocyclyl-C₀-C₃alkyl-, —C₀-C₃alkyl-N(R₃)—C(S)-heterocyclyl-C₀-C₃alkyl-, —C₀-C₇alkyl-heterocyclyl-C₀-C₃alkyl-, —C₀-C₃alkyl-O—C(O)-heterocyclyl-C₀-C₃alkyl-, C₀-C₃alkyl-S(O)₂-heterocyclyl-C₀-C₃alkyl- and —C₀-C₃alkyl-C(O)-heterocyclyl-C₀-C₃alkyl, wherein said alkyl and heterocyclyl are independently optionally subsituted; and
Y is selected from the group consisting of heteroaryl, aryl, cycloalkyl and heteroaryl-aryl, each of which is optionally substituted.

64. The compound according to claim 1, wherein
W is nitrogen;
X is —O—;
R₁ and R₂ are H;
Q is optionally substituted alkyl-aryl;
Z is optionally substituted —C₀-C₆alkyl-heteroaryl-C₀-C₆alkyl and optionally substituted C₁-C₈alkyl;
L is selected from the group consisting of —C₀-C₆alkyl-S(O)₂-heterocyclyl-C₀-C₃alkyl, covalent bond, —C₀-C₆alkyl-, —C₀-C₆alkyl-O—C(O)—N(R₃)—C₀-C₃alkyl-, —C₀-C₆alkyl-C(O)—O—, —C₀-C₆alkyl-N(R₃)—C(O)-heterocyclyl-C₀-C₃alkyl-, —C₀-C₃alkyl-heteroaryl-C₀-C₃alkyl-N(R₃)—CO—C₃alkyl-, —C₀-C₆alkyl-N(R₃)—C(O)—N(R₃)—C₀-C₃alkyl-, —C₀-C₆alkyl-C(O)—N(R₃)—C₀-C₃alkyl-, —C₀-C₆alkyl-S(O)₂—N(R₃)—C₀-C₃alkyl-, —C₀-C₆alkyl-C(O)—N(R₃)—C(O)—N(R₃)—C₀-C₆alkyl- and —C₀-C₆alkyl-S(O)₂—C₀-C₃alkyl, wherein the alkyl, heterocyclyl, heteroaryl are independently optionally substituted; and
Y is selected from the group consisting of aryl, alkylaryl, heteroaryl, aryl-heterocyclyl, aryl-heteroaryl, alkyl and heterocyclyl, each of which is independently optionally substituted.

65. The compound according to claim 1, wherein
W is nitrogen;
X is —O—;
R₁ and R₂ are H;
Q is optionally substituted alkyl-aryl;
Z is selected from the group consisting of —C₀-C₆alkyl-aryl-C₀-C₆alkyl-, —C₀-C₆alkyl-aryl-C₀-C₃alkyl-X—C₀-C₃alkyl-, —C₀-C₆alkyl-aryl-C₃-C₆alkenyl-C₀-C₃alkyl and —C₀-C₆alkyl-aryl-C₃-C₆alkynyl-C₀-C₃alkyl, wherein the alkyl, aryl and alkynyl are each independently optionally substituted;
L is selected from the group consisting of —C₀-C₃alkyl-N(R₃)—C₀-C₃alkyl, —C₀-C₃alkyl-heterocyclyl-C₀-C₃alkyl-O—C₀-C₃alkyl-, —C₀-C₆alkyl-S(O)₂—N(R₃)—C₀-C₆alkyl-, —C₀-C₆alkyl-O—C(O)—, —C₀-C₆alkyl-C(O)—N(R₃)—S(O)₂—C₀-C₃alkyl- and —C₀-C₆alkyl-N(R₃)—S(O)₂—C₀-C₃alkyl, wherein said alkyl and heterocyclyl are each independently optionally substituted; and
Y is selected from the group consisting of heteroaryl, aryl, heteroaryl-hetercyclyl, alkyl, aryl-heterocyclyl and cycloalky, each of which is independently optionally substituted.

66. The compound according to claim 1, wherein
W is nitrogen;
X is —O—;
R₁ and R₂ are H;
Q is optionally substituted alkyl-aryl;
Z is optionally substituted C₁-C₆alkyl;
L is —C₀-C₃alkyl-N(R₃)—C(O)—C₀-C₃alkyl-heterocyclyl-C(O)—C₀-C₃alkyl, wherein the alkyl and heterocyclyl are independently optionally substituted; and
Y is selected from the group consisting of aryl-aryl, alkyl-heteroaryl, aryl and heteroaryl, each of which is independently optionally substituted.

67. The compound according to claim 1, wherein 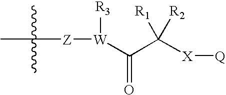
is the structure

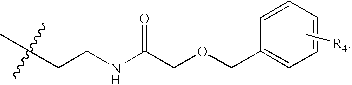

68. A compound according to claim 1 that is selected from the group consisting of
2-(2-(6-(biphenyl-3-ylamino)-6-oxohexylamino)-2-oxoethylthio)acetic acid,
N-(biphenyl-3-yl)-6-(2-(4-(methylthio)benzylthio)acetamido)hexanamide,
methyl 3-(2-(6-(biphenyl-3-ylamino)-6-oxohexylamino)-2-oxoethylthio)-2-hydroxypropanoate,
methyl 4-(2-(6-(biphenyl-3-ylamino)-6-oxohexylamino)-2-oxoethylthio)butanoate,
methyl 2-(2-(6-(biphenyl-3-ylamino)-6-oxohexylamino)-2-oxoethylthio)acetate,
(S)-tert-butyl 6-(2-(benzyloxy)acetamido)-1-oxo-1-(quinolin-8-ylamino)hexan-2-ylcarbamate;
N-(biphenyl-3-yl)-6-(2-(2-oxo-2-(phenylamino)ethylthio)acetamido)hexanamide,
2-(2-(6-(biphenyl-3-ylamino)-6-oxohexylamino)-2-oxoethylsulfinyl)acetic acid,
methyl 2-(2-(2-(biphenyl-3-ylamino)-2-oxoethylamino)-2-oxoethylsulfonyl)acetate,
6-(2-(2-aminoethoxy)acetamido)-N-(biphenyl-3-yl)hexanamide,
N-(biphenyl-3-yl)-6-(2-(4-fluorobenzyloxy)ethanethioamido)hexanamide,
4-((2-(6-(biphenyl-3-ylamino)-6-oxohexylamino)-2-oxoethoxy)methyl)benzoic acid,
N-(biphenyl-3-yl)-6-(2-(4-(hydroxymethyl)benzyloxy)acetamido)hexanamide,
N-(biphenyl-3-yl)-6-(2-(4-cyanobenzyloxy)-acetamido)-hexanamide,
N-(biphenyl-3-yl)-6-(2-(4-methylbenzyloxy)-acetamido)-hexanamide,
N-(biphenyl-3-yl)-6-(2-(thiophen-2-ylmethoxy)-acetamido)-hexanamide,
N-(biphenyl-3-yl)-6-(2-(thiophen-3-ylmethoxy)-acetamido)-hexanamide,
N-(biphenyl-3-yl)-6-(2-(furan-3-ylmethoxy)-acetamido)-hexanamide,
N-(biphenyl-3-yl)-6-(2-(4-bromobenzyloxy)-acetamido)-hexanamide,
N-(biphenyl-3-yl)-6-(2-(naphthalen-1-ylmethoxy)-acetamido)-hexanamide,
N-(biphenyl-3-yl)-6-(2-(2-oxo-2-(phenylamino)-ethylsulfinyl)-acetamido)-hexanamide,
3-(2-(6-(biphenyl-3-ylamino)-6-oxohexylamino)-2-oxoethylthio)-propanoic acid,
methyl 3-(2-(6-(biphenyl-3-ylamino)-6-oxohexylamino)-2-oxoethylsulfinyl)-propanoate,
N-(biphenyl-3-yl)-6-(2-(3-hydroxypropylthio)-acetamido)-hexanamide,
N-(biphenyl-3-yl)-6-(2-(2-hydroxyethylthio)-acetamido)-hexanamide,
6-(2-(2-amino-2-oxoethylthio)-acetamido)-N-(biphenyl-3-yl)hexanamide,
(R)-ethyl 2-amino-3-(2-(6-(biphenyl-3-ylamino)-6-oxohexylamino)-2-oxoethylthio)-propanoate,
(R)-2-amino-3-(2-(6-(biphenyl-3-ylamino)-6-oxohexylamino)-2-oxoethylthio)-propanoic acid,
6-(2-(2-aminoethylthio)-acetamido)-N-(biphenyl-3-yl)hexanamide,
N-(biphenyl-3-yl)-6-(2-(4-hydroxyphenylthio)-acetamido)-hexanamide,
methyl 2-(2-(6-(biphenyl-3-ylamino)-6-oxohexylamino)-2-oxoethylsulfinyl)-acetate,
N-(biphenyl-3-yl)-6-(2-(3-oxo-3-(phenylamino)-propylthio)-acetamido)-hexanamide,
N-(biphenyl-3-yl)-6-(2-(3-oxo-3-(phenylamino)-propylsulfinyl)-acetamido)-hexanamide,
N-(biphenyl-3-yl)-6-(2-(3-hydroxyphenylthio)-acetamido)-hexanamide,
6-(2-(4-aminophenylthio)-acetamido)-N-(biphenyl-3-yl)hexanamide,
N-(biphenyl-3-yl)-6-(2-(4-fluorophenylthio)-acetamido)-hexanamide,
N-(biphenyl-3-yl)-6-(2-(phenylthio)-acetamido)-hexanamide,
N-(biphenyl-3-yl)-6-(2-(4-chlorophenylthio)-acetamido)-hexanamide,
N-(biphenyl-3-yl)-6-(2-(4-bromophenylthio)-acetamido)-hexanamide,
6-(2-(3-aminophenylthio)-acetamido)-N-(biphenyl-3-yl)hexanamide,
N-(biphenyl-3-yl)-6-(2-(pyridin-4-ylthio)acetamido)-hexanamide,
N-(biphenyl-3-yl)-6-(2-(thiophen-2-ylthio)acetamido)-hexanamide,
N-(biphenyl-3-yl)-6-(2-(4-(methylthio)benzyloxy)acetamido)hexanamide),
N-(biphenyl-3-yl)-6-(2-(naphthalen-2-ylmethoxy)-acetamido)-hexanamide,
N-(biphenyl-3-yl)-6-(2-(4-fluorobenzyloxy)-acetamido)-hexanamide,
N-(biphenyl-3-yl)-6-(2-(4-chlorobenzyloxy)-acetamido)-hexanamide,
N-(biphenyl-3-yl)-6-(2-(pyridin-4-ylmethoxy)-acetamido)-hexanamide,
N-(biphenyl-3-yl)-6-(2-(pyridin-3-ylmethoxy)-acetamido)-hexanamide,
N-(biphenyl-3-yl)-6-(2-(4-(methylsulfinyl)-benzyloxy)-acetamido)-hexanamide,
6-(2-(benzylthio)acetamido)-N-(biphenyl-3-yl)hexanamide,
6-(2-(benzylsulfinyl)acetamido)-N-(biphenyl-3-yl)hexanamide,
N-(biphenyl-3-yl)-6-(4-(thiophen-2-yl)butanamido)-hexanamide,
N-(biphenyl-3-yl)-6-(3-(4-fluorobenzylthio)propanamido)hexanamide,
N-(biphenyl-3-yl)-6-(2-(4-fluorobenzyl-thio)acetamido)-hexanamide,
N-(biphenyl-3-yl)-6-(2-(4-fluorobenzyl-sulfinyl)-acetamido)-hexanamide,
2-(3-(6-(biphenyl-3-ylamino)-6-oxohexylamino)-3-oxopropylthio)-acetic acid,
2-(3-(6-(biphenyl-3-ylamino)-6-oxohexylamino)-3-oxopropyl-sulfinyl)acetic acid,
6-(2-(benzyloxy)acetamido)-N-(biphenyl-3-yl)hexanamide,
(S)-2-amino-6-(2-(benzyloxy)acetamido)hexanoic acid,
(S)-benzyl 6-(2-(benzyloxy)acetamido)-1-oxo-1-(quinolin-8-ylamino)hexan-2-ylcarbamate,
(S)-2-(2-oxo-2-(4-((3-oxo-2-(thiophen-2-ylmethyl)-3,4-dihydroquinoxalin-1(2H)-yl)methyl)phenylamino)ethylthio)acetic acid,
N-(biphenyl-3-yl)-6-(2-(2-(pyridin-2-yl)ethylthio)acetamido)hexanamide,
N-(biphenyl-3-yl)-6-(2-(2-(diethylamino)ethylthio)acetamido)hexanamide,
4-(2-(6-(biphenyl-3-ylamino)-6-oxohexylamino)-2-oxoethylthio)butanoic acid,
N-(biphenyl-3-yl)-6-(2-(4-(methylsulfinyl)benzylthio)acetamido)hexanamide,
N-(biphenyl-3-yl)-6-(2-(2-(dimethylamino)ethylthio)-acetamido)-hexanamide,
(S)-3-(2-(4-((2-benzyl-3-oxo-3,4-dihydroquinoxalin-1(2H)-yl)methyl)phenylamino)-2-oxoethylthio)propanoic acid,
(R)-3-(2-oxo-2-(4-((3-oxo-2-(thiophen-2-ylmethyl)-3,4-dihydroquinoxalin-1(2H)-yl)methyl)-phenylamino-)ethylthio)-propanoic acid,
3-(2-(6-methoxybenzo[d]thiazol-2-ylamino)-2-oxoethylthio)propanoic acid,
4-(6-(biphenyl-3-ylamino)-6-oxohexylamino)-2-hydroxy-4-oxobutanoic acid,
3-(2-(6-(biphenyl-3-ylamino)-6-oxohexylamino)-2-oxoethylthio)-2-hydroxypropanoic acid,
N-(4-(((2-(1H-indol-3-yl)ethyl)(2-hydroxyethyl)amino)methyl)benzyl)-2-(3-hydroxypropylthio)acetamide,
(R)-N-(4-((2-((1H-indol-3-yl)methyl)-3-oxo-3,4-dihydroquinoxalin-1(2H)-yl)methyl)phenyl)-2-(2-(dimethylamino)ethylthio)acetamide,
3-(2-(benzo[d]thiazol-2-ylamino)-2-oxoethylthio)propanoic acid,
3-(2-(6-fluorobenzo[d]-thiazol-2-ylamino)-2-oxoethylthio)-propanoic acid,
3-({2-[(6-nitro-1,3-benzothiazol-2-yl)amino]-2-oxoethyl}thio)propanoic acid,
3-(2-(6-nitrobenzo[d]-thiazol-2-ylamino)-2-oxoethylthio)-propanoic acid,
3-(2-oxo-2-(6-oxo-6-(3-(pyridin-3-yl)phenylamino)hexylamino)ethylthio)-propanoic acid,
N-biphenyl-3-yl-6-({[(4-fluorobenzyl)oxy]acetyl}amino)hexanamide,
2-(4-aminophenylthio)-N-(4-(biphenyl-4-ylsulfonamido)phenethyl)acetamide,
benzyl 4-(2-(2-(4-aminophenylthio)acetamido)ethyl)phenylcarbamate,
2-(4-aminophenylthio)-N-(2-(4-(biphenyl-4-ylsulfonamido)phenylthio)ethyl)acetamide,
N-(3-(4-(N-(3,4-dimethoxyphenyl)-N-methylsulfamoyl)phenyl)propyl)-2-(4-fluorobenzyloxy)acetamide,
N-(3-(4-(N-(3,4-dimethoxyphenyl)sulfamoyl)phenyl)propyl)-2-(4-fluorobenzyloxy)acetamide,
N-(3-(4-(N-(3,4-dimethoxyphenyl)sulfamoyl)phenyl)propyl)-2-(pyridin-4-ylthio)acetamide,
2-(4-aminophenylthio)-N-(6-(4-(4-methoxyphenyl)piperazin-1-yl)-6-oxohexyl)acetamide,
2-(4-fluorophenylthio)-N-(6-(4-(4-methoxyphenyl)piperazin-1-yl)-6-oxohexyl)acetamide,
6-(2-(4-aminophenylthio)acetamido)-N-phenylhexanamid,
6-(2-(4-aminophenylthio)acetamido)-N-(pyridin-3-yl)hexanamide,
2-(4-aminophenylthio)-N-(4-(biphenyl-4-ylsulfonamido)butyl)acetamide,
2-(4-aminophenylthio)-N-(5-(biphenyl-4-ylsulfonamido)pentyl)acetamide,
5-(2-(4-aminobenzyloxy)acetamido)-N-(biphenyl-3-yl)pentanamide,
N-(biphenyl-3-yl)-5-(2-(4-fluorobenzyloxy)acetamido)pentanamide,
4-((2-(6-(biphenyl-3-ylamino)-6-oxohexylamino)-2-oxoethoxy)methyl)pyridine 1-oxide,
4-(2-(6-(biphenyl-3-ylamino)-6-oxohexylamino)-2-oxoethylsulfonyl)pyridine 1-oxide,
5-(2-(5-aminopyridin-2-ylthio)acetamido)-N-(biphenyl-3-yl)pentanamide,
6-(2-(5-aminopyridin-2-ylthio)acetamido)-N-(biphenyl-3-yl)hexanamide,
5-methoxy-N-(5-(2-(thiophen-2-ylthio)acetamido)pentyl)-1H-indole-2-carboxamide,
N-(5-(2-(4-aminophenylthio)acetamido)pentyl)-4-(dimethylamino)benzamide,
5-chloro-N-(5-(2-(4-fluorobenzyloxy)acetamido)pentyl)-1H-indole-2-carboxamide,
5-fluoro-N-(5-(2-(4-fluorobenzyloxy)acetamido)pentyl)-1H-indole-2-carboxamide,
(S)-benzyl 1-(5-(2-(4-fluorobenzyloxy)acetamido)pentylamino)-1-oxo-3-phenylpropan-2-yl(methyl)carbamate,
N-(5-(2-(4-aminophenylthio)acetamido)pentyl)-1H-indole-2-carboxamide,
N-(2-(5-(biphenyl-4-ylsulfonamido)-1,3,4-thiadiazol-2-ylthio)ethyl)-2-(4-fluorobenzyloxy)acetamide,
N-(3-(5-(3,4-dimethoxyphenylsulfonamido)-1,3,4-thiadiazol-2-ylthio)propyl)-2-(4-fluorobenzyloxy)acetamide,
N-(4-(5-(biphenyl-4-ylsulfonamido)-1,3,4-thiadiazol-2-ylthio)butyl)-2-(4-fluorobenzyloxy)acetamide,
2-(4-fluorobenzyloxy)-N-(3-(4-(4-methoxyphenyl)pyrimidin-2-ylthio)propyl)acetamide,
2-(4-fluorobenzyloxy)-N-(3-(4-(4-methoxyphenyl)pyrimidin-2-ylsulfinyl)propyl)acetamide,
N-(5-(5-(4-bromophenyl)-1,3,4-thiadiazol-2-yl)pentyl)-2-(4-fluorobenzyloxy)acetamide,
N-(4-(N-(cyclohexylcarbamoyl)sulfamoyl)phenethyl)-2-(4-fluorobenzyloxy)acetamide,
3-(2-(3-(4-(N-(3,4-dimethoxyphenyl)sulfamoyl)phenyl)propylamino)-2-oxoethylthio)propanoic,
N-(3-(4-(N-(3,4-dimethoxyphenyl)sulfamoyl)phenyl)prop-2-ynyl)-2-(pyridin-4-ylmethoxy)acetamide,
N-(3-(4-(N-(3,4-dimethoxyphenyl)sulfamoyl)phenyl)prop-2-ynyl)-2-(pyridin-4-ylthio)acetamide,
N-(3-(4-(N-(2-(1H-indol-3-yl)ethyl)sulfamoyl)phenyl)propyl)-2-(4-fluorobenzyloxy)acetamide,
N-(3-(4-(N-(2-(1H-indol-3-yl)ethyl)-N-(2-hydroxyethyl)sulfamoyl)phenyl)propyl)-2-(4-fluorobenzyloxy)acetamide,
N-(3-(4-(N-(3,4-dimethoxybenzyl)sulfamoyl)phenyl)propyl)-2-(4-fluorobenzyloxy)acetamide,
N-(4-(N-(3,4-dimethoxyphenyl)sulfamoyl)phenethyl)-2-(4-fluorobenzyloxy)acetamide,
N-(3-(4-(3,4-dimethoxyphenylsulfonamido)phenyl)propyl)-2-(4-fluorobenzyloxy)acetamide,
pyridin-3-ylmethyl 4-(3-(2-(4-fluorobenzyloxy)acetamido)propyl)phenylcarbamate,
pyridin-3-ylmethyl 4-(3-(2-(4-fluorobenzyloxy)acetamido)propyl)benzylcarbamate,
N-(3-(4-(2-(2-(1H-indol-3-yl)ethylamino)ethyl)phenyl)propyl)-2-(4-fluorobenzyloxy)acetamide,
(S)-2-(4-fluorobenzyloxy)-N-(3-(4-((1-hydroxy-3-(1H-indol-3-yl)propan-2-ylamino)methyl)phenyl)propyl)acetamide,
(S)-2-(4-fluorobenzyloxy)-N-(3-(4-(((1-hydroxy-3-(1H-indol-3-yl)propan-2-yl)(2-hydroxyethyl)amino)methyl)phenyl)propyl)acetamide,
2-(4-fluorobenzyloxy)-N-(3-(4-((2-(5-methoxy-1H-indol-3-yl)ethylamino)methyl)phenyl)propyl)acetamide,
N-(3-(4-((2-(5-(benzyloxy)-1H-indol-3-yl)ethylamino)methyl)phenyl)propyl)-2-(4-fluorobenzyloxy)acetamide,
N-(3-(4-((2-(5-fluoro-1H-indol-3-yl)ethylamino)methyl)phenyl)propyl)-2-(4-fluorobenzyloxy)acetamide,
N-(3-(4-((2-(1H-indol-3-yl)ethylamino)methyl)phenyl)propyl)-2-(4-fluorobenzyloxy)acetamide,
2-(4-fluorobenzyloxy)-N-(3-(4-(((2-hydroxyethyl)(2-(5-methoxy-1H-indol-3-yl)ethyl)amino)methyl)phenyl)propyl)acetamide,
N-(3-(4-(((2-(5-(benzyloxy)-1H-indol-3-yl)ethyl)(2-hydroxyethyl)amino)methyl)phenyl)propyl)-2-(4-fluorobenzyloxy)acetamide,
N-(3-(4-(((2-(5-fluoro-1H-indol-3-yl)ethyl)(2-hydroxyethyl)amino)methyl)phenyl)propyl)-2-(4-fluorobenzyloxy)acetamide,
N-(3-(4-(((2-(1H-indol-3-yl)ethyl)(cyclohexyl)amino)methyl)phenyl)propyl)-2-(4-fluorobenzyloxy)acetamide,
N-(3-(4-(((2-(1H-indol-3-yl)ethyl)(tetrahydro-2H-pyran-4-yl)amino)methyl)phenyl)propyl)-2-(4-fluorobenzyloxy)acetamide,
N-(3-(4-(((2-(1H-indol-3-yl)ethyl)(3-hydroxypropyl)amino)methyl)phenyl)propyl)-2-(4-fluorobenzyloxy)acetamide,
N-(3-(4-(((2-(1H-indol-3-yl)ethyl)(2,3-dihydroxypropyl)amino)methyl)phenyl)propyl)-2-(4-fluorobenzyloxy)acetamide,
N-(3-(4-((2-(benzo[d][1,3]dioxol-5-yl)ethylamino)methyl)phenyl)propyl)-2-(4-fluorobenzyloxy)acetamide,
N-(3-(4-((2-(4-benzylpiperidin-1-yl)ethylamino)methyl)phenyl)propyl)-2-(4-fluorobenzyloxy)acetamide,
N-(3-(4-(((2-(benzo[d][1,3]dioxol-5-yl)ethyl)(2-hydroxyethyl)amino)methyl)phenyl)propyl)-2-(4-fluorobenzyloxy)acetamide,
N-(3-(4-(((2-(4-benzylpiperidin-1-yl)ethyl)(2-hydroxyethyl)amino)methyl)phenyl)propyl)-2-(4-fluorobenzyloxy)acetamide,
N-(3-(4-((2-(1H-benzo[d]imidazol-2-yl)ethylamino)methyl)phenyl)propyl)-2-(4-fluorobenzyloxy)acetamide,
N-(3-(4-(((2-(1H-benzo[d]imidazol-2-yl)ethyl)(2-hydroxyethyl)amino)methyl)phenyl)propyl)-2-(4-fluorobenzyloxy)acetamide,
2-(4-fluorobenzyloxy)-N-(3-(4-(((2-hydroxyethyl)(2-(1-(2-hydroxyethyl)-1H-benzo[d]imidazol-2-yl)ethyl)amino)methyl)phenyl)propyl)acetamide,
N-(3-(4-(((2-(1H-indol-3-yl)ethyl)(methyl)amino)methyl)phenyl)propyl)-2-(4-fluorobenzyloxy)acetamide,
N-(3-(4-((2-(benzofuran-3-yl)ethylamino)methyl)phenyl)propyl)-2-(4-fluorobenzyloxy)acetamide,
N-(3-(4-(((2-(benzofuran-3-yl)ethyl)(2-hydroxyethyl)amino)methyl)phenyl)propyl)-2-(4-fluorobenzyloxy)acetamide,
N-(3-(4-(((1H-indol-3-yl)methylamino)methyl)phenyl)propyl)-2-(4-fluorobenzyloxy)acetamide,
N-(3-(4-((((1H-indol-3-yl)methyl)(2-hydroxyethyl)amino)methyl)phenyl)propyl)-2-(4-fluorobenzyloxy)acetamide,
N-(3-(4-(((5-fluoro-1H-indol-3-yl)methylamino)methyl)phenyl)propyl)-2-(4-fluorobenzyloxy)acetamide,
N-(3-(4-((((5-fluoro-1H-indol-3-yl)methyl)(2-hydroxyethyl)amino)methyl)phenyl)propyl)-2-(4-fluorobenzyloxy)acetamide,
N-(biphenyl-3-yl)-6-(2,2-difluoro-2-(4-fluorophenylthio)acetamido)hexanamide,
N-(5-(4,5-diphenyl-1H-imidazol-1-yl)pentyl)-2-(4-fluorobenzyloxy)acetamide,
2-(4-fluorobenzyloxy)-N-(3-(3-(piperidin-1-ylmethyl)phenoxy)propyl)acetamide,
N-(3-(3-((4-benzylpiperidin-1-yl)methyl)phenoxy)propyl)-2-(4-fluorobenzyloxy)acetamide,
2-(4-fluorobenzyloxy)-N-(3-(3-((4-phenylpiperidin-1-yl)methyl)phenoxy)propyl)acetamide,
N-(3-(3-((3,4-dihydroisoquinolin-2(1H)-yl)methyl)phenoxy)propyl)-2-(4-fluorobenzyloxy)acetamide,
N-(3-(3-((4-benzylpiperazin-1-yl)methyl)phenoxy)propyl)-2-(4-fluorobenzyloxy)acetamide,
N-(4-(3-((4-benzylpiperidin-1-yl)methyl)phenoxy)butyl)-2-(4-fluorobenzyloxy)acetamide,
N-(4-(3-((2-(1H-indol-3-yl)ethylamino)methyl)phenoxy)butyl)-2-(4-fluorobenzyloxy)acetamide,
N-(5-(6-(bis(pyridin-3-ylmethyl)amino)benzo[d]thiazol-2-yl)pentyl)-2-(4-fluorobenzyloxy)acetamide,
N-(5-(6-(3,4-dimethoxyphenylsulfonamido)benzo[d]thiazol-2-yl)pentyl)-2-(4-fluorobenzyloxy)acetamide,
N-(3-(3-(N-(3,4-dimethoxyphenyl)sulfamoyl)phenyl)propyl)-2-(4-fluorobenzyloxy)acetamide,
(E)-2-(4-chlorobenzyloxy)-N-(3-(4-(N-(3,4-dimethoxyphenyl)sulfamoyl)phenyl)allyl)acetamide,
2-(4-chlorobenzyloxy)-N-(3-(4-(N-(3,4-dimethoxyphenyl)sulfamoyl)phenyl)propyl)acetamide,
N-(biphenyl-3-yl)-6-(2-(2-hydroxy-1-phenylethylthio)acetamido)hexanamide,
N-(4-(3-(2-(4-fluorobenzyloxy)acetamido)propyl)benzyl)-2-(phenylmethylsulfonamido)benzamide,
N-(3-(4-(((2-(1H-indol-3-yl)ethyl)(2-hydroxyethyl)amino)methyl)phenyl)propyl)-2-(4-fluorobenzyloxy)acetamide,
N-(3,4-dimethoxyphenyl)-4-(3-(1-oxoisoindolin-2-yl)propyl)benzenesulfonamide,
N-(biphenyl-3-yl)-6-(2-(2-cyanoethylthio)acetamido)hexanamide,
2-(4-aminophenylthio)-N-(5-(5-(4-(pyridin-3-yl)phenyl)oxazol-2-yl)pentyl)acetamide,
2-(4-fluorophenylthio)-N-(5-(2-phenylthiazol-4-yl)pentyl)acetamide,
2-(4-fluorophenylthio)-N-(5-(2-(pyridin-3-yl)thiazol-4-yl)pentyl)acetamide,
2-(4-fluorophenylthio)-N-(5-(2-(pyridin-3-yl)thiazol-4-yl)pentyl)acetamide,
(S)-N-(5-(2-(4-fluorobenzyloxy)acetamido)-1-(5-phenyl-1,3,4-thiadiazol-2-yl)pentyl)nicotinamide,
(S)-2-(dimethylamino)-N-(5-(2-(4-fluorobenzyloxy)acetamido)-1-(5-phenyl-1,3,4-thiadiazol-2-yl)pentyl)acetamide,
(S)-benzyl 6-(2-(4-aminobenzyloxy)acetamido)-1-oxo-1-(quinolin-8-ylamino)hexan-2-ylcarbamate,
(R)-benzyl 6-(2-(4-aminobenzyloxy)acetamido)-1-oxo-1-(quinolin-8-ylamino)hexan-2-ylcarbamate,
allyl(S)-1-((S)-1-(allylamino)-3-(1-methyl-1H-indol-3-yl)-1-oxopropan-2-ylamino)-6-(2-(4-aminophenylthio)acetamido)-1-oxohexan-2-ylcarbamate,
N-(4-((4S,7R,E)-7-benzyl-2,5,8-trioxo-1-oxa-3,6,9-triazacyclotetradec-12-en-4-yl)butyl)-2-(4-fluorobenzyloxy)acetamide,
(S)-N-(4-(2,5-dioxo-1,2,3,4,5,6,7,8,9,10-decahydrobenzo[b][1,4,7]oxadiazacyclotridecin-3-yl)butyl)-2-(4-fluorobenzyloxy)acetamide,
2-(4-fluorobenzyloxy)-N-(3-(4-((2-(5-sulfamoyl-1H-indol-3-yl)ethylamino)methyl)phenyl)propyl)acetamide,
N-(3-(4-(((2-(7-fluoro-1H-indol-3-yl)ethyl)(2-hydroxyethyl)amino)methyl)phenyl)propyl)-2-(4-fluorobenzyloxy)acetamide,
(S)-tert-butyl 4-(2-(5-(1H-benzo[d]imidazol-2-yl)-5-(4-fluorobenzamido)pentylamino)-2-oxoethylthio)phenylcarbamate,
(S)-N-(5-acetamido-5-(1H-benzo[d]imidazol-2-yl)pentyl)-2-(4-fluorophenylthio)acetamide,
(R)-N-(1-(1H-benzo[d]imidazol-2-yl)-5-(2-(4-fluorophenylthio)acetamido)pentyl)-4-fluorobenzamide,
(S)-N-(1-(5-chloro-6-fluoro-1H-benzo[d]imidazol-2-yl)-5-(2-(4-fluorophenylthio)acetamido)pentyl)-4-fluorobenzamide,
(S)-N-(5-(2-(4-aminophenylthio)acetamido)-1-(1H-benzo[d]imidazol-2-yl)pentyl)nicotinamide,
(S)-N-(5-(2-(4-aminophenylthio)acetamido)-1-(1H-benzo[d]imidazol-2-yl)pentyl)benzamide,
(S)-N-(5-(2-(4-aminophenylthio)acetamido)-1-(1H-benzo[d]imidazol-2-yl)pentyl)-4-(dimethylamino)benzamide,
N-(5-(1-(3,4-dimethoxyphenethyl)-1H-benzo[d]imidazol-2-yl)pentyl)-2-(4-fluorobenzyloxy)acetamide,
2-(4-fluorobenzyloxy)-N-(5-(1-methyl-1H-benzo[d]imidazol-2-yl)pentyl)acetamide,
2-(4-fluorobenzyloxy)-N-(4-(1-phenyl-1H-benzo[d]imidazol-2-yl)butyl)acetamide,
2-(4-fluorobenzyloxy)-N-(5-(1-(4-sulfamoylphenethyl)-1H-benzo[d]imidazol-2-yl)pentyl)acetamide,
N-(5-(1H-benzo[d]imidazol-2-yl)pentyl)-2-(4-fluorobenzyloxy)acetamide,
(S)-N-(2-(1H-indol-3-yl)ethyl)-1-(6-(2-(4-fluorobenzyloxy)acetamido)hexanoyl)pyrrolidine-2-carboxamide,
(S)—N-(biphenyl-4-ylmethyl)-1-(6-(2-(4-fluorobenzyloxy)acetamido)hexanoyl)pyrrolidine-2-carboxamide,
(S)—N-(biphenyl-4-yl)-1-(6-(2-(4-fluorobenzyloxy)acetamido)hexanoyl)pyrrolidine-2-carboxamide,
(S)-1-(6-(2-(4-fluorobenzyloxy)acetamido)hexanoyl)-N-(pyridin-3-ylmethyl)pyrrolidine-2-carboxamide,
(S)-N-(2-aminophenyl)-1-(6-(2-(4-fluorobenzyloxy)acetamido)hexanoyl)pyrrolidine-2-carboxamide,
(S)-N-(6-(2-(1H-benzo[d]imidazol-2-yl)pyrrolidin-1-yl)-6-oxohexyl)-2-(4-fluorobenzyloxy)acetamide,
2-(4-fluorobenzyloxy)-N-(4-((2S,5S)-5-((1-methyl-1H-indol-3-yl)methyl)-3,6,12-trioxo-1,4,7-triazacyclododecan-2-yl)butyl)acetamide,
N-(4-((3S,6R,9S,14aR)-9-sec-butyl-1,4,7,10-tetraoxo-6-(4-(trifluoromethyl)benzyl)-tetradecahydropyrrolo[1,2-a][1,4,7,10]tetraazacyclododecin-3-yl)butyl)-2-(4-fluorobenzyloxy)acetamide,
2-(4-fluorobenzyloxy)-N-(5-(4-phenyl-1H-1,2,3-triazol-1-yl)pentyl)acetamide,
2-(4-fluorobenzyloxy)-N-(5-(4-(pyridin-3-yl)-1H-1,2,3-triazol-1-yl)pentyl)acetamide,
N-(4-(3,4-dimethoxyphenylsulfonamido)phenethyl)-2-(4-fluorophenylthio)acetamide,
2-(4-aminophenylthio)-N-(4-(3,4-dimethoxyphenylsulfonamido)phenethyl)acetamide,
2-(4-aminobenzyloxy)-N-(4-(3,4-dimethoxyphenylsulfonamido)phenethyl)acetamide,
(R)-2-(5-aminopyridin-2-ylthio)-N-(4-((3-oxo-2-(thiophen-2-ylmethyl)-3,4-dihydroquinoxalin-1(2H)-yl)methyl)phenyl)acetamide,
N-(2-(4-(((2-(1H-indol-3-yl)ethyl)(2-hydroxyethyl)amino)methyl)phenoxy)ethyl)-2-(4-fluorobenzyloxy)acetamide,
2-((2-(1H-indol-3-yl)ethyl)(4-(2-(2-(4-fluorobenzyloxy)acetamido)ethoxy)benzyl)amino)acetic acid,
N-(3-(4-((4-(1H-indol-3-yl)piperidin-1-yl)methyl)phenyl)propyl)-2-(4-fluorobenzyloxy)acetamide,
2-(4-fluorobenzyloxy)-N-(3-(4-((6-methoxy-3,4-dihydro-1H-pyrido[3,4-b]indol-2(9H)-yl)methyl)phenyl)propyl)acetamide,
6-(2-(4-aminobenzyloxy)acetamido)-N-(pyridin-3-yl)hexanamide,
N-(biphenyl-3-yl)-6-(2-(4-(hydroxymethyl)phenylthio)acetamido)hexanamide,
2-(4-fluorobenzyloxy)-N-(6-(4-(4-methoxyphenyl)piperazin-1-yl)-6-oxohexyl)acetamide,
N-(biphenyl-3-yl)-6-(2-(furan-2-ylmethylthio)acetamido)hexanamide,
N-(biphenyl-3-yl)-6-(2-(furan-2-ylmethylsulfinyl)acetamido)hexanamide,
2-(4-fluorobenzyloxy)-N-(2-(pyridin-3-yl)ethyl)acetamide,
2-(4-fluorobenzyloxy)-N-(2-(4-(4-methoxyphenyl)pyrimidin-2-ylthio)ethyl)acetamide,
2-(4-fluorobenzyloxy)-N-(2-(4-(thiophen-2-yl)pyrimidin-2-ylthio)ethyl)acetamide,
2-(4-fluorobenzyloxy)-N-(2-(4-(4-methoxyphenyl)pyrimidin-2-ylsulfinyl)ethyl)acetamide,
2-(4-fluorobenzyloxy)-N-(4-(4-(4-methoxyphenyl)pyrimidin-2-ylthio)butyl)acetamide,
8-(2-(2-aminophenyl)hydrazinyl)-N-(biphenyl-3-yl)-8-oxooctanamide,
N-(biphenyl-3-yl)-8-oxo-8-(2-phenylhydrazinyl)octanamide,
6-(2-(allyloxy)acetamido)-N-(biphenyl-3-yl)hexanamide,
methyl 3-(2-(6-(biphenyl-3-ylamino)-6-oxohexylamino)-2-oxoethylthio)-3-phenylpropanoate,
ethyl 3-(2-(6-(biphenyl-3-ylamino)-6-oxohexylamino)-2-oxoethylthio)-3-phenylpropanoate,
ethyl 3-(2-(6-(biphenyl-3-ylamino)-6-oxohexylamino)-2-oxoethylsulfinyl)-3-phenylpropanoate,
ethyl 2-(2-oxo-2-(5-(5-phenyl-1,3,4-thiadiazol-2-yl)pentylamino)ethylthio)-2-phenylacetate,
ethyl 2-(2-oxo-2-(6-oxo-6-(quinolin-8-ylamino)hexylamino)ethylthio)-2-phenylacetate,
N-(3-(4-(3,4-dimethoxybenzylsulfonyl)phenyl)propyl)-2-(4-fluorobenzyloxy)acetamide,
ethyl 2-(2-(3-(4-(N-(3,4-dimethoxyphenyl)sulfamoyl)phenyl)propylamino)-2-oxoethylthio)-2-phenylacetate,
ethyl 2-(2-(3-(4-(N-(3,4-dimethoxyphenyl)sulfamoyl)phenyl)propylamino)-2-oxoethylthio)-2-phenylacetate,
N-(3-(4-(N-(3,4-dimethoxyphenyl)sulfamoyl)phenyl)propyl)benzofuran-2-carboxamide,
N-(3-(3-(((2-(1H-indol-3-yl)ethyl)(2-hydroxyethyl)amino)methyl)phenoxy)propyl)-2-(4-fluorobenzyloxy)acetamide,
2-(4-aminophenylthio)-N-(5-(5-phenylthiazol-2-yl)pentyl)acetamide,
2-(4-aminophenylthio)-N-(5-(5-(4-methoxyphenyl)thiazol-2-yl)pentyl)acetamide,
2-(4-fluorobenzyloxy)-N-(5-(5-phenyl-1,3,4-thiadiazol-2-yl)pentyl)acetamide,
N-(4-(5-(3,4-dimethoxyphenylsulfonamido)-1,3,4-thiadiazol-2-ylthio)butyl)-2-(4-fluorobenzyloxy)acetamide,
N-(4-(3-((3-(3,4-dimethoxyphenylsulfonamido)pyrrolidin-1-yl)methyl)phenoxy)butyl)-2-(4-fluorobenzyloxy)acetamide,
pyridin-3-ylmethyl 1-(3-(4-(2-(4-fluorobenzyloxy)acetamido)butoxy)benzyl)pyrrolidin-3-ylcarbamate,
N-(1-(3-(4-(2-(4-fluorobenzyloxy)acetamido)butoxy)benzyl)pyrrolidin-3-yl)isonicotinamide,
1-(3-(4-(2-(4-fluorobenzyloxy)acetamido)butoxy)benzyl)pyrrolidin-3-yl pyridin-3-ylmethylcarbamate,
(E)-N-(3-(4-(((2-(1H-indol-3-yl)ethyl)(2-hydroxyethyl)amino)methyl)phenyl)allyl)-2-(4-fluorobenzyloxy)acetamide,
(S)-benzyl 1-oxo-6-(2-(pyridin-4-ylthio)acetamido)-1-(quinolin-8-ylamino)hexan-2-ylcarbamate,
(R)-benzyl 1-oxo-6-(2-(pyridin-4-ylthio)acetamido)-1-(quinolin-8-ylamino)hexan-2-ylcarbamate,
(S)-benzyl 6-(2-(4-aminophenylthio)acetamido)-1-oxo-1-(quinolin-8-ylamino)hexan-2-ylcarbamate,
(R)-benzyl 6-(2-(4-aminophenylthio)acetamido)-1-oxo-1-(quinolin-8-ylamino)hexan-2-ylcarbamate,
N-(10,11-dihydro-5H-dibenzo[a,a]cyclohepten-5-yl)-6-(2-(4-fluorobenzyloxy)acetamido)-hexanamide,
(S,E/Z (7:3))-N-(4-(2,5-dioxo-14-phenyl-1,2,3,4,5,6,7,10-octahydrobenzo[b][1,4,7]oxadiazacyclotridecin-3-yl)butyl)-2-(4-fluorobenzyloxy)acetamide,
(S)-N-(4-(2,5-dioxo-14-phenyl-1,2,3,4,5,6,7,8,9,10-decahydrobenzo[b][1,4,7]oxadiazacyclotridecin-3-yl)butyl)-2-(4-fluorobenzyloxy)acetamide,
N-(3-(4-((3-(2-(1H-indol-3-yl)ethyl)ureido)methyl)phenyl)propyl)-2-(4-fluorobenzyloxy)acetamide,
2-(1H-indol-3-yl)ethyl 4-(3-(2-(4-fluorobenzyloxy)acetamido)propyl)benzylcarbamate,
2-(4-fluorobenzyloxy)-N-(3-(4-((2-(2-methyl-1H-indol-3-yl)ethylamino)methyl)phenyl)prop-2-ynyl)acetamide,
2-(4-fluorobenzyloxy)-N-(3-(4-((2-(2-methyl-1H-indol-3-yl)ethylamino)methyl)phenyl)propyl)acetamide,
(S)-N-(1-(5-chloro-6-fluoro-1H-benzo[d]imidazol-2-yl)-5-(2-(4-fluorophenylthio)acetamido)pentyl)-4-fluorobenzamide,
N-(5-(1H-benzo[d]imidazol-2-yl)pentyl)-2-(4-fluorophenylthio)acetamide,
N,N-bis(2-(1H-indazol-5-ylamino)-2-oxoethyl)-6-(2-(4-fluorobenzyloxy)acetamido)hexanamide,
N,N-bis(2-(1H-indazol-5-ylamino)-2-oxoethyl)-5-(2-(4-fluorobenzyloxy)acetamido)pentanamide,
N-(4-(1-benzhydrylazetidin-3-yl)butyl)-2-(4-fluorobenzyloxy)acetamide,
N-(4-(-(3,4-dimethoxyphenylsulfonyl)azetidin-3-yl)butyl)-2-(4-fluorobenzyloxy)acetamide,
tert-butyl 3-(4-(2-(4-fluorobenzyloxy)acetamido)butyl)azetidine-1-carboxylate,
N-(2-(1H-indol-3-yl)ethyl)-3-(4-(2-(4-fluorobenzyloxy)acetamido)butyl)azetidine-1-carboxamide,
3-(4-(2-(4-fluorobenzyloxy)acetamido)butyl)-N-((1-methyl-1H-benzo[d]imidazol-2-yl)methyl)azetidine-1-carboxamide,
N-(4-(1-(2-(1H-indol-3-yl)ethylcarbamothioyl)azetidin-3-yl)butyl)-2-(4-fluorobenzyloxy)acetamide,
N-(2-(1H-indol-3-yl)ethyl)-3-(4-(2-(4-fluorobenzyloxy)acetamido)butyl)pyrrolidine-1-carboxamide,
3-(4-(2-(4-fluorobenzyloxy)acetamido)butyl)-N-((1-methyl-1H-benzo[d]imidazol-2-yl)methyl)pyrrolidine-1-carboxamide,
2-(1H-indol-3-yl)ethyl 3-(4-(2-(4-fluorobenzyloxy)acetamido)butyl)pyrrolidine-1-carboxylate,
N-(4-(1-((1H-indol-3-yl)methyl)pyrrolidin-3-yl)butyl)-2-(4-fluorobenzyloxy)-acetamide,
2-(4-fluorobenzyloxy)-N-(4-(1-((5-methoxy-1H-indol-2-yl)methyl)pyrrolidin-3-yl)butyl)acetamide,
N-(4-(1-((5-fluoro-1H-indol-2-yl)methyl)pyrrolidin-3-yl)butyl)-2-(4-fluorobenzyloxy)acetamide,
N-(3-(-(3,4-dimethoxyphenylsulfonyl)pyrrolidin-3-yl)propyl)-2-(4-fluorobenzyloxy)acetamide,
4-(2-(2-(4-fluorobenzyloxy)acetamido)ethyl)-N-(3,4-dimethoxyphenyl)piperidine-1-carboxamide,
N-(2-(1H-indol-3-yl)ethyl)-4-(2-(2-(4-fluorobenzyloxy)acetamido)ethyl)piperidine-1-carboxamide,
N-cyclohexyl-4-(2-(2-(4-fluorobenzyloxy)acetamido)ethyl)piperidine-1-carboxamide,
4-(2-(2-(4-fluorobenzyloxy)acetamido)ethyl)-N-(quinolin-8-yl)piperidine-1-carboxamide,
4-(2-(2-(4-fluorobenzyloxy)acetamido)ethyl)-N-(2-(5-methoxy-1H-indol-3-yl)ethyl)piperidine-1-carboxamide,
4-(2-(2-(4-fluorobenzyloxy)acetamido)ethyl)-N-(4-(dimethylamino)phenyl)piperidine-1-carboxamide,
4-(2-(2-(4-fluorobenzyloxy)acetamido)ethyl)-N-(1-methyl-1H-benzo[d]imidazol-2-yl)piperidine-1-carboxamide,
(S)-methyl 2-(4-(2-(2-(4-fluorobenzyloxy)acetamido)ethyl)piperidine-1-carboxamido)-3-(1H-indol-3-yl)propanoate,
N-(2-(1H-benzo[d]imidazol-2-yl)ethyl)-4-(2-(2-(4-fluorobenzyloxy)acetamido)ethyl)piperidine-1-carboxamide,
4-(2-(2-(4-fluorobenzyloxy)acetamido)ethyl)-N-((1-methyl-1H-benzo[d]imidazol-2-yl)methyl)piperidine-1-carboxamide,
4-(2-(2-(4-fluorobenzyloxy)acetamido)ethyl)-N-(2-(2-methyl-1H-indol-3-yl)ethyl)piperidine-1-carboxamide,
4-(2-(2-(4-fluorobenzyloxy)acetamido)ethyl)-N-phenylpiperidine-1-carboxamide,
ethyl 4-(4-(2-(2-(4-fluorobenzyloxy)acetamido)ethyl)piperidine-1-carboxamido)benzoate,
N-(2-(benzo[d][1,3]dioxol-5-yl)ethyl)-4-(2-(2-(4-fluorobenzyloxy)acetamido)ethyl)piperidine-1-carboxamide,
N-(3,5-dimethylisoxazol-4-yl)-4-(2-(2-(4-fluorobenzyloxy)acetamido)ethyl)piperidine-1-carboxamide,
4-(2-(2-(4-fluorobenzyloxy)acetamido)ethyl)-N-(3-methyl-5-phenylisoxazol-4-yl)piperidine-1-carboxamide,
4-(2-(2-(4-fluorobenzyloxy)acetamido)ethyl)-N-(2-(6-methoxy-1H-indol-3-yl)ethyl)piperidine-1-carboxamide,
N-(2-(5-fluoro-1H-indol-3-yl)ethyl)-4-(2-(2-(4-fluorobenzyloxy)acetamido)ethyl)piperidine-1-carboxamide,
N-(3-(1H-indol-3-yl)propyl)-4-(2-(2-(4-fluorobenzyloxy)acetamido)ethyl)piperidine-1-carboxamide,
2-(1H-indol-3-yl)ethyl 4-(2-(2-(4-fluorobenzyloxy)acetamido)ethyl)piperidine-1-carboxylate,
pyridin-3-ylmethyl 4-(2-(2-(4-fluorobenzyloxy)acetamido)ethyl)piperidine-1-carboxylate,
N-(2-(1H-indol-2-yl)ethyl)-4-(2-(2-(4-fluorobenzyloxy)acetamido)ethyl)-N-(2-hydroxyethyl)piperidine-1-carboxamide,
(S)-4-(2-(2-(4-fluorobenzyloxy)acetamido)ethyl)-N-(1-hydroxy-3-(1H-indol-3-yl)propan-2-yl)piperidine-1-carboxamide,
4-(4-(2-(2-(4-fluorobenzyloxy)acetamido)ethyl)piperidine-1-carboxamido)benzoic acid,
N-(2-(1-(3,4-dimethoxyphenylsulfonyl)piperidin-4-yl)ethyl)-2-(4-fluorobenzyloxy)acetamide,
N-(2-(1-(benzylsulfonyl)piperidin-4-yl)ethyl)-2-(4-fluorobenzyloxy)acetamide,
4-(3-(2-(4-fluorobenzyloxy)acetamido)propyl)-N-(3,4-dimethoxyphenyl)piperidine-1-carboxamide,
N-(2-(1-(2-(4-(dimethylamino)phenyl)acetyl)piperidin-4-yl)ethyl)-2-(4-fluorobenzyloxy)acetamide,
N-(2-(1-(2-(1H-indol-3-yl)acetyl)piperidin-4-yl)ethyl)-2-(4-fluorobenzyloxy)acetamide,
N-(2-(1-(2-(1H-indol-3-yl)ethyl)piperidin-4-yl)ethyl)-2-(4-fluorobenzyloxy)acetamide,
N-(2-(1-(3-(1H-indol-3-yl)propyl)piperidin-4-yl)ethyl)-2-(4-fluorobenzyloxy)acetamide,
2-(4-fluorobenzyloxy)-N-(2-(1-((1-methyl-1H-indol-3-yl)methyl)piperidin-4-yl)ethyl)acetamide,
2-(4-fluorobenzyloxy)-N-(5-(2-(4-methoxyphenyl)thiazol-4-yl)pentyl)acetamide,
N-(5-(2-(2-aminophenyl)thiazol-4-yl)pentyl)-2-(4-fluorobenzyloxy)acetamide,
N-(5-(2-benzhydrylthiazol-4-yl)pentyl)-2-(4-fluorobenzyloxy)acetamide,
N-(5-(2-(4-bromophenyl)thiazol-4-yl)pentyl)-2-(4-fluorobenzyloxy)acetamide,
N-(5-(2-(4-chlorobenzyl)thiazol-4-yl)pentyl)-2-(4-fluorobenzyloxy)acetamide,
N-(5-(2-(3-bromothiophen-2-yl)thiazol-4-yl)pentyl)-2-(4-fluorobenzyloxy)acetamide,
N-(5-(2-(4-(1H-pyrrol-1-yl)phenyl)thiazol-4-yl)pentyl)-2-(4-fluorobenzyloxy)acetamide,
2-(4-fluorobenzyloxy)-N-(5-(2-phenylthiazol-4-yl)pentyl)acetamide,
2-(4-fluorobenzyloxy)-N-(5-(2-(3-hydroxyphenyl)thiazol-4-yl)pentyl)acetamide,
2-(4-fluorobenzyloxy)-N-(5-(2-(4-hydroxyphenyl)thiazol-4-yl)pentyl)acetamide,
N-(5-(2-(benzo[d][1,3]dioxol-5-yl)thiazol-4-yl)pentyl)-2-(4-fluorobenzyloxy)acetamide,
2-(4-fluorobenzyloxy)-N-(5-(2-(4-morpholinophenyl)thiazol-4-yl)pentyl)acetamide,
N-(5-(2-(4-(1H-imidazol-1-yl)phenyl)thiazol-4-yl)pentyl)-2-(4-fluorobenzyloxy)acetamide,
N-(5-(2-(benzo[b]thiophen-3-yl)thiazol-4-yl)pentyl)-2-(4-fluorobenzyloxy)acetamide,
N-(5-(2-(2,3-dihydrobenzo[b][1,4]dioxin-2-yl)thiazol-4-yl)pentyl)-2-(4-fluorobenzyloxy)acetamide,
2-(4-fluorobenzyloxy)-N-(5-(2′-methyl-2,4′-bithiazol-4-yl)pentyl)acetamide,
N-(5-(2-(1H-imidazol-4-yl)thiazol-4-yl)pentyl)-2-(4-fluorobenzyloxy)acetamide,
tert-butyl(4-(5-(2-(4-fluorobenzyloxy)acetamido)pentyl)thiazol-2-yl)methylcarbamate,
(4-(5-(2-(4-fluorobenzyloxy)acetamido)pentyl)thiazol-2-yl)methyl pivalate,
tert-butyl 4-(4-(5-(2-(4-fluorobenzyloxy)acetamido)pentyl)thiazol-2-yl)piperidine-1-carboxylate,
N-(5-(2-((2-(1H-indol-3-yl)ethylamino)methyl)thiazol-4-yl)pentyl)-2-(4-fluorobenzyloxy)acetamide,
4-(4-(5-(2-(4-fluorobenzyloxy)acetamido)pentyl)thiazol-2-yl)-N-phenylpiperidine-1-carboxamide,
N-(3,4-dimethoxyphenyl)-4-(4-(5-(2-(4-fluorobenzyloxy)acetamido)pentyl)thiazol-2-yl)piperidine-1-carboxamide,
2-(4-fluorobenzyloxy)-N-(5-(2-(1-(phenylsulfonyl)piperidin-4-yl)thiazol-4-yl)pentyl)acetamide,
N-(5-(2-(1-(3,4-dimethoxyphenylsulfonyl)piperidin-4-yl)thiazol-4-yl)pentyl)-2-(4-fluorobenzyloxy)acetamide,
2-(4-fluorobenzyloxy)-N-(5-(2-(1-(4-fluorophenylsulfonyl)piperidin-4-yl)thiazol-4-yl)pentyl)acetamide,
2-(4-fluorobenzyloxy)-N-(5-(4-(2-hydroxyphenyl)thiazol-2-ylamino)pentyl)acetamide,
2-(4-fluorobenzyloxy)-N-(6-(4-(2-hydroxyphenyl)thiazol-2-ylamino)hexyl)acetamide,
N-(5-(2-aminothiazol-4-yl)pentyl)-2-(4-fluorobenzyloxy)acetamide,
2-(4-fluorobenzyloxy)-N-(5-(2-(phenylsulfonamido)thiazol-4-yl)pentyl)acetamide,
N-(5-(2-(3,4-dimethoxyphenylsulfonamido)thiazol-4-yl)pentyl)-2-(4-fluorobenzyloxy)acetamide,
N-(5-(2-(3,5-dimethylisoxazol-4-sulfonamido)thiazol-4-yl)pentyl)-2-(4-fluorobenzyloxy)acetamide,
2-(4-fluorobenzyloxy)-N-(5-(2-(phenylmethylsulfonamido)thiazol-4-yl)pentyl)acetamide,
N-(5-(2-(1,2-dimethyl-1H-imidazole-4-sulfonamido)thiazol-4-yl)pentyl)-2-(4-fluorobenzyloxy)acetamide,
2-(4-fluorobenzyloxy)-N-(5-(2-(3-(1-(2,2,2-trifluoroacetyl)piperidin-4-yl)ureido)thiazol-4-yl)pentyl)acetamide,
2-(4-fluorobenzyloxy)-N-(5-(2-(3-phenylureido)thiazol-4-yl)pentyl)acetamide,
N-(5-(2-(3-(3,4-dimethoxyphenyl)ureido)thiazol-4-yl)pentyl)-2-(4-fluorobenzyloxy)acetamide,
N-(4-(5-(2-(4-fluorobenzyloxy)acetamido)pentyl)thiazol-2-ylcarbamoyl)benzamide,
N-(5-(2-(3-(3,5-dimethylisoxazol-4-yl)ureido)thiazol-4-yl)pentyl)-2-(4-fluorobenzyloxy)acetamide,
methyl 3-(3-(4-(5-(2-(4-fluorobenzyloxy)acetamido)pentyl)thiazol-2-yl)ureido)benzoate,
2-(4-fluorobenzyloxy)-N-(5-(2-(3-propylureido)thiazol-4-yl)pentyl)acetamide,
2-(4-fluorobenzyloxy)-N-(5-(2-(3-p-tolylureido)thiazol-4-yl)pentyl)acetamide,
2-(4-fluorobenzyloxy)-N-(5-(2-(3-(3-methoxyphenyl)ureido)thiazol-4-yl)pentyl)acetamide,
2-(4-fluorobenzyloxy)-N-(5-(2-(3-(4-fluorophenyl)ureido)thiazol-4-yl)pentyl)acetamide,
N-(5-(2-(3-benzylureido)thiazol-4-yl)pentyl)-2-(4-fluorobenzyloxy)acetamide,
2-(4-fluorobenzyloxy)-N-(5-(2-(3-piperidin-4-ylureido)thiazol-4-yl)pentyl)acetamide hydrochloride,
N-(4-(5-(2-(4-fluorobenzyloxy)acetamido)pentyl)thiazol-2-yl)benzo[d][1,3]dioxole-5-carboxamide,
N-(5-(2-acetamidothiazol-4-yl)pentyl)-2-(4-fluorobenzyloxy)acetamide,
N-(4-(5-(2-(4-fluorobenzyloxy)acetamido)-pentyl)thiazol-2-yl)-3,4-dimethoxybenzamide,
N-(4-(5-(2-(4-fluorobenzyloxy)acetamido)pentyl)thiazol-2-yl)benzamide,
Phenyl 4-(5-(2-(4-fluorobenzyloxy)acetamido)-pentyl)thiazol-2-ylcarbamate,
2-(4-fluorobenzyloxy)-N-(5-(3-(4-fluorophenyl)-1,2,4-oxadiazol-5-yl)pentyl)acetamide,
N-(5-(3-(4-fluoro-3-methoxyphenyl)-1,2,4-oxadiazol-5-yl)pentyl)-2-(4-fluorobenzyloxy)acetamide,
2-(4-fluorobenzyloxy)-N-(5-(3-(3,4,5-trimethoxyphenyl)-1,2,4-oxadiazol-5-yl)pentyl)acetamide,
benzyl(5-(5-(2-(4-fluorobenzyloxy)acetamido)pentyl)-1,2,4-oxadiazol-3-yl)methylcarbamate,
N-(5-(3-(4-(dimethylamino)phenyl)-1,2,4-oxadiazol-5-yl)pentyl)-2-(4-fluorobenzyloxy)acetamide,
N-(3-(6-(3,4-dimethoxyphenylsulfonamido)pyridin-3-yl)propyl)-2-(4-fluorobenzyloxy)acetamide,
2-(4-fluorobenzyloxy)-N-(3-(4-(3-methoxyphenylsulfonamido)phenyl)propyl)acetamide,
2-(4-fluorobenzyloxy)-N-(3-(4-(4-methoxyphenylsulfonamido)phenyl)propyl)acetamide,
N-(3-(4-(2-acetamido-4-methylthiazole-5-sulfonamido)phenyl)propyl)-2-(4-fluorobenzyloxy)acetamide,
2-(4-fluorobenzyloxy)-N-(3-(4-(6-morpholinopyridine-3-sulfonamido)phenyl)propyl)acetamide,
N-(3-(4-(benzo[d][1,3]dioxole-5-sulfonamido)phenyl)propyl)-2-(4-fluorobenzyloxy)-acetamide,
2-(4-fluorobenzyloxy)-N-(3-(4-(phenylmethylsulfonamido)phenyl)propyl)acetamide,
N-(3-(4-(3,5-dimethylisoxazole-4-sulfonamido)phenyl)propyl)-2-(4-fluorobenzyloxy)acetamide,
N-(3-(4-(3,4-dimethoxyphenylsulfonamido)phenyl)propyl)-2-(4-fluorobenzyloxy)acetamide,
N-(3-(4-(2,3-dihydrobenzo[b][1,4]dioxine-6-sulfonamido)phenyl)propyl)-2-(4-fluorobenzyloxy)acetamide,
2-(4-fluorobenzyloxy)-N-(3-(4-(4-methyl-3,4-dihydro-2H-benzo[b][1,4]oxazine-7-sulfonamido)phenyl)propyl)acetamide,
2-(4-fluorobenzyloxy)-N-(3-(4-(phenylsulfonamido)phenyl)propyl)acetamide,
(R)-tert-butyl 3-(4-(3-(2-(4-fluorobenzyloxy)acetamido)propyl)phenoxy)pyrrolidine-1-carboxylate,
(S)-tert-butyl 3-(4-(3-(2-(4-fluorobenzyloxy)acetamido)propyl)phenoxy)pyrrolidine-1-carboxylate,
R)—N-(3-(4-(1-((1H-indol-3-yl)methyl)pyrrolidin-3-yloxy)phenyl)propyl)-2-(4-fluorobenzyloxy)acetamide,
(S)-N-(3-(4-(1-((1H-indol-3-yl)methyl)pyrrolidin-3-yloxy)phenyl)propyl)-2-(4-fluorobenzyloxy)acetamide,
(R)-N-(3-(4-(1-(2-(1H-indol-3-yl)ethyl)pyrrolidin-3-yloxy)phenyl)propyl)-2-(4-fluorobenzyloxy)acetamide,
(S)-N-(3-(4-(1-(2-(1H-indol-3-yl)ethyl)pyrrolidin-3-yloxy)phenyl)propyl)-2-(4-fluorobenzyloxy)acetamide,
N-(4-(3-(2-(4-fluorobenzyloxy)acetamido)propyl)phenylsulfonyl)-3,4-dimethoxybenzamide,
N-(3-(4-(3,4-dimethoxyphenylsulfonamido)phenoxy)propyl)-2-(4-fluorobenzyloxy)acetamide,
N-(4-(4-(3,4-dimethoxyphenylsulfonamido)phenoxy)butyl)-2-(4-fluorobenzy-loxy)acetamide,
6-(2-(4-aminophenylthio)acetamido)-N-(quinolin-2-yl)hexanamide,
6-(2-(4-aminophenylthio)acetamido)-N-(4-phenoxyphenyl)hexanamide,
6-(2-(4-aminophenylthio)acetamido)-N-(4-(4-chlorophenyl)thiazol-2-yl)hexanamide,
6-(2-(4-aminophenylthio)acetamido)-N-(naphthalen-2-yl)hexanamide,
6-(2-(4-aminophenylthio)acetamido)-N-(biphenyl-4-ylmethyl)hexanamide,
6-(2-(4-aminophenylthio)acetamido)-N-(benzo[d]thiazol-6-yl)hexanamide,
6-(2-(4-aminophenylthio)acetamido)-N-(quinolin-3-yl)hexanamide dihydrochloride,
6-(2-(4-aminophenylthio)acetamido)-N-(4-phenylthiazol-2-yl)hexanamide hydrochloride,
6-(2-(4-aminophenylthio)acetamido)-N-(quinolin-8-yl)hexanamide dihydrochloride,
6N-((1H-benzo[d]imidazol-2-yl)methyl)-6-(2-(4-aminophenylthio)acetamido)hexanamide dihydrochloride,
6-(2-(4-aminophenylthio)acetamido)-N-(quinolin-6-yl)hexanamide dihydrochloride,
6-(2-(4-aminophenylthio)acetamido)-N-(benzo[d]thiazol-2-yl)hexanamide trifluoroacetic acid,
6-(2-(4-aminophenylthio)acetamido)-N-(6-methoxybenzo[d]thiazol-2-yl)hexanamide hydrochloride,
6-(2-(4-aminophenylthio)acetamido)-N-(4,6-difluorobenzo[d]thiazol-2-yl)hexanamide trifluoroacetic acid,
6-(2-(4-aminophenylthio)acetamido)-N-(4-(4-methoxyphenyl)thiazol-2-yl)hexanamide hydrochloride,
(6-(2-(4-fluorophenylthio)acetamido)-N-(4-(4-(2-morpholinoethoxy)phenyl)thiazol-2-yl)hexanamide),
N-(3-(4-(N-benzo[d][1,3]dioxol-5-ylsulfamoyl)phenyl)propyl)-2-(4-fluorobenzyloxy)acetamide,
2-(4-fluorobenzyloxy)-N-(3-(4-(N-(4-methoxyphenyl)sulfamoyl)phenyl)propyl)acetamide,
2-(4-fluorobenzyloxy)-N-(3-(4-(N-phenylsulfamoyl)phenyl)propyl)acetamide,
2-(4-fluorobenzyloxy)-N-(3-(4-(N-(3-methoxyphenyl)sulfamoyl)phenyl)propyl)acetamide,
2-(4-fluorobenzyloxy)-N-(3-(4-(N-(4-(4-methylpiperazin-1-yl)phenyl)sulfamoyl)phenyl)propyl)acetamide,
N-(3-(4-(N-(2,3-dihydrobenzo[b][1,4]dioxin-6-yl)sulfamoyl)phenyl)propyl)-2-(4-fluorobenzyloxy)acetamide,
2-(4-fluorobenzyloxy)-N-(3-(4-(N-(4-(4-methylpiperazin-1-yl)phenyl)sulfamoyl)phenyl)propyl)acetamide,
N-(3-(4-(N-(3,5-dimethoxyphenyl)sulfamoyl)phenyl)propyl)-2-(4-fluorobenzyloxy)acetamide,
N-(3-(4-(N-cyclohexylsulfamoyl)phenyl)propyl)-2-(4-fluorobenzyloxy)acetamide,
methyl 4-(4-(3-(2-(4-fluorobenzyloxy)acetamido) propyl)phenylsulfonamido) benzoate,
2-(4-fluorobenzyloxy)-N-(3-(4-(N-(3-hydroxy-4-methoxyphenyl)sulfamoyl)phenyl)propyl)acetamide,
N-(3-(4-(N-(3,4-dihydro-2H-benzo[b][1,4]dioxepin-7-yl)sulfamoyl)phenyl)propyl)-2-(4-fluorobenzyloxy)acetamide,
N-(3-(4-(N-(2,3-dimethoxyphenyl)sulfamoyl)phenyl)propyl)-2-(4-fluorobenzyloxy)acetamide,
2-(4-fluorobenzyloxy)-N-(3-(4-(N-(3-oxo-3,4-dihydro-2H-benzo[b][1,4]oxazin-7-yl)sulfamoyl)phenyl)-propyl)acetamide,
2-(4-fluorobenzyloxy)-N-(3-(4-(N-(4-hydroxy-3-methoxyphenyl)sulfamoyl)phenyl)propyl)acetamide,
2-(4-fluorobenzyloxy)-N-(3-(4-(N-(3,4,5-trimethoxyphenyl)sulfamoyl)phenyl)prop-2-ynyl)acetamide,
2-(4-fluorobenzyloxy)-N-(3-(4-(N-(5-(trifluoromethyl)-1,3,4-thiadiazol-2-yl)sulfamoyl)phenyl)prop-2-ynyl)acetamide,
2-(4-fluorobenzyloxy)-N-(3-(4-(N-(3-hydroxy-4-methoxyphenyl)sulfamoyl)phenyl)prop-2-ynyl)acetamide,
N-(3-(4-(N-(3,5-dimethoxyphenyl)sulfamoyl)phenyl)prop-2-ynyl)-2-(4-fluorobenzyloxy)acetamide,
N-(3-(4-(N-cyclohexylsulfamoyl)phenyl)prop-2-ynyl)-2-(4-fluorobenzyl-oxy)acetamide,
(Z)-2-(4-fluorobenzyloxy)-N-(3-(4-(N-(3,4,5-trimethoxyphenyl)sulfamoyl)phenyl)allyl)acetamide,
N-(3-(4-(N-(3-(2-(dimethylamino)ethoxy)-4-methoxyphenyl)sulfamoyl)phenyl)propyl)-2-(4-fluorobenzyloxy)acetamide,
N-(3-(4-(N-(4-(2-(dimethylamino)ethoxy)-3-methoxyphenyl)sulfamoyl)phenyl)propyl)-2-(4-fluorobenzyloxy)acetamide,
N-(3-(4-(N-(4-amino-3-methoxyphenyl)sulfamoyl)phenyl)propyl)-2-(4-fluorobenzyloxy)acetamide,
(E)-N-(4-(4-(N-(3,4-dimethoxyphenyl)sulfamoyl)phenyl)but-3-enyl)-2-(4-fluorobenzyloxy)acetamide,
(E)-N-(4-(4-(N-(2,3-dihydrobenzo[b][1,4]dioxin-6-yl)sulfamoyl)phenyl)but-3-enyl)-2-(4-fluorobenzyloxy)acetamide,
N-(4-(4-(N-(3,4-dimethoxyphenyl)sulfamoyl)phenyl)butyl)-2-(4-fluorobenzyloxy)acetamide,
N-(4-(N-(2,3-dihydrobenzo[b][1,4]dioxin-6-yl)sulfamoyl)phenethyl)-2-(4-fluorobenzyloxy)acetamide,
N-(4-(N-(3,5-dimethoxyphenyl)sulfamoyl)phenethyl)-2-(4-fluorobenzyloxy)acetamide,
N-(4-(4-(N-(2,3-dihydrobenzo[b][1,4]dioxin-6-yl)sulfamoyl)phenyl)butyl)-2-(4-fluorobenzyloxy)acetamide,
N-(4-(4-(N-(3-fluoro-4-methoxyphenyl)sulfamoyl)phenyl)butyl)-2-(4-fluorobenzyloxy)acetamide,
2-(4-fluorobenzyloxy)-N-(4-(4-(N-(3,4,5-trimethoxyphenyl)sulfamoyl)phenyl)butyl)acetamide,
N-(5-(1-(3,4-dimethoxyphenylsulfonyl)-1H-pyrrol-3-yl)pentyl)-2-(4-fluorobenzyloxy)acetamide,
N-(4-(((2-(1H-indol-3-yl)ethyl)(2-hydroxyethyl)amino)methyl)phenethyl)-2-(4-fluorobenzyloxy)acetamide,
N-(4-((2-(1H-indol-3-yl)ethylamino)methyl)phenethyl)-2-(4-fluorobenzyloxy)acetamide,
4-(3-(2-(4-fluorobenzyloxy)acetamido)prop-1-ynyl)benzenesulfonic acid,
N-(4-(3-(1H-benzo[d]imidazol-2-yl)azetidin-1-yl)butyl)-2-(4-fluorobenzyloxy)acetamide,
3-(2-(4-(3-(2-(4-fluorobenzyloxy)acetamido)propyl)benzylamino)ethyl)-1H-indol-5-yl sulfite,
N-(3-(4-((2-(7-fluoro-1H-indol-3-yl)ethylamino)methyl)phenyl)propyl)-2-(4-fluorobenzyloxy)acetamide,
3-(2-(4-(3-(2-(4-fluorobenzyloxy)acetamido)propyl)benzylamino)ethyl)-1H-indol-5-yl sulfite and
N-(3-(4-((2-(7-fluoro-1H-indol-3-yl)ethylamino)methyl)phenyl)propyl)-2-(4-fluorobenzyloxy)acetamide, or
a pharmaceutically acceptable salt thereof.

69. A composition comprising a compound according to claim 1 and a pharmaceutically acceptable carrier.

70. A method of inhibiting histone deacetylase, the method comprising contacting the histone deacetylase with an inhibiting effective amount of a compound according to claim 1.

71. A method of inhibiting histone deacetylase, the method comprising contacting the histone deacetylase with an inhibiting effective amount of a composition according to claim 69.

72. A method of inhibiting histone deacetylase in a cell, the method comprising contacting the cell with an inhibiting effective amount of compound according to claim 1.

73. A method of inhibiting histone deacetylase in a cell, the method comprising contacting the cell with an inhibiting effective amount of a composition according to claim 69.
